SEQUENCE LISTING (566 sequences)

SEQ ID NO: 1 - protein, 787 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSEDSSALPWSINRDDYELQEVIGSGATAVVQAAYCAPKKEKVAIKRINLEKCQTSMDELLKEIQAMSQCHHPNIVSYYTSFVVKDELWLVMKLLSGGSVLDIIKHIVAKGEHKSGVLDESTIATILREVLEGLEYLHKNGQIHRDVKAGNILLGEDGSVQIADFGVSAFLATGGDITRNKVRKTFVGTPCWMAPEVMEQVRGYDFKADIWSFGITAIELATGAAPYHKYPPMKVLMLTLQNDPPSLETGVQDKEMLKKYGKSFRKMISLCLQKDPEKRPTAAELLRHKFFQKAKNKEFLQEKTLQRAPTISERAKKVRRVPGSSGRLHKTEDGGWEWSDDEFDEESEEGKAAISQLRSPRVKESISNSELFPTTDPVGTLLQVPEQISAHLPQPAGQIATQPTQVSLPPTAEPAKTAQALSSGSGSQETKIPISLVLRLRNSKKELNDIRFEFTPGRDTAEGVSQELISAGLVDGRDLVIVAANLQKIVEEPQSNRSVTFKLASGVEGSDIPDDGKLIGFAQLSIS

SEQ ID NO: 2 - protein, 527 aa, Homo sapiens
MSEDSSALPWSINRDDYELQEVIGSGATAVVQAAYCAPKKEKVAIKRINLEKCQTSMDELLKEIQAMSQCHHPNIVSYYTSFVVKDELWLVMKLLSGGSVLDIIKHIVAKGEHKSGVLDESTIATILREVLEGLEYLHKNGQIHRDVKAGNILLGEDGSVQIADFGVSAFLATGGDITRNKVRKTFVGTPCWMAPEVMEQVRGYDFKADIWSFGITAIELATGAAPYHKYPPMKVLMLTLQNDPPSLETGVQDKEMLKKYGKSFRKMISLCLQKDPEKRPTAAELLRHKFFQKAKNKEFLQEKTLQRAPTISERAKKVRRVPGSSGRLHKTEDGGWEWSDDEFDEESEEGKAAISQLRSPRVKESISNSELFPTTDPVGTLLQVPEQISAHLPQPAGQIATQPTQVSLPPTAEPAKTAQALSSGSGSQETKIPISLVLRLRNSKKELNDIRFEFTPGRDTAEGVSQELISAGLVDGRDLVIVAANLQKIVEEPQSNRSVTFKLASGVEGSDIPDDGKLIGFAQLSIS

SEQ ID NO: 3 - protein, 607 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASGILVNVKEEVTCPICLELLTQPLSLDCGHSFCQACLTANHKKSMLDKGESSCPVCRISYQPENIRPNRHVANIVEKLREVKLSPEGQKVDHCARHGEKLLLFCQEDGKVICWLCERSQEHRGHHTFLTEEVAREYQVKLQAALEMLRQKQQEAEELEADIREEKASWKTQIQYDKTNVLADFEQLRDILDWEESNELQNLEKEEEDILKSLTNSETEMVQQTQSLRELISDLEHRLQGSVMELLQGVDGVIKRTENVTLKKPETFPKNQRRVFRAPDLKGMLEVFRELTDVRRYWGKEKSHYHKPPCGLSLLLSLSFRILCSLLGSCFKIYDSPSKTHITYPSL

SEQ ID NO: 4 - protein, 347 aa, Homo sapiens
MASGILVNVKEEVTCPICLELLTQPLSLDCGHSFCQACLTANHKKSMLDKGESSCPVCRISYQPENIRPNRHVANIVEKLREVKLSPEGQKVDHCARHGEKLLLFCQEDGKVICWLCERSQEHRGHHTFLTEEVAREYQVKLQAALEMLRQKQQEAEELEADIREEKASWKTQIQYDKTNVLADFEQLRDILDWEESNELQNLEKEEEDILKSLTNSETEMVQQTQSLRELISDLEHRLQGSVMELLQGVDGVIKRTENVTLKKPETFPKNQRRVFRAPDLKGMLEVFRELTDVRRYWGKEKSHYHKPPCGLSLLLSLSFRILCSLLGSCFKIYDSPSKTHITYPSL

SEQ ID NO: 5 - protein, 607 aa, synthetic construct; identical to SEQ ID NO: 3

SEQ ID NO: 6 - protein, 347 aa, Homo sapiens; identical to SEQ ID NO: 4

SEQ ID NO: 7 - protein, 528 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAALGASGGAGAGDDDFDQFDKPGAERSWRRRAADEDWDSELEDDLLGEDLLSGKKNQSDLSDEELNDDLLQSDNEDEENFSSQGVTISLNATSGMVTSFELSDNTNDQSGEQESEYEQEQGEDELVYHKSDGSELYTQEYPEEGQYEGHEAELTEDQIEYVEEPEEEQLYTDEVLDIEINEPLDEFTGGMETLELQKDIKEESDEEEEDDEESGRLRFKTERKEGTIIRLSDVTRERRNIPETLGNFFACLPSSFTLISTSSIVLL

SEQ ID NO: 8 - protein, 268 aa, Homo sapiens
MAAALGASGGAGAGDDDFDQFDKPGAERSWRRRAADEDWDSELEDDLLGEDLLSGKKNQSDLSDEELNDDLLQSDNEDEENFSSQGVTISLNATSGMVTSFELSDNTNDQSGEQESEYEQEQGEDELVYHKSDGSELYTQEYPEEGQYEGHEAELTEDQIEYVEEPEEEQLYTDEVLDIEINEPLDEFTGGMETLELQKDIKEESDEEEEDDEESGRLRFKTERKEGTIIRLSDVTRERRNIPETLGNFFACLPSSFTLISTSSIVLL

SEQ ID NO: 9 - protein, 577 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPTSNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 10 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPTSNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 11 - protein, 829 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAPTLATAHRRRWWMACTAVLENLLFSAVLLGWGSLLIMLKSEGFYSYLCTEPENVTNGTVGGTAEPGHEEVSWMNGWLSCQAQDEMLNLAFTVGSFLLSAITLPLGIVMDKYGPRKLRLLGSACFAVSCLLIAYGASKPNALSVLIFIALALNGFGGMCMTFTSLTLPNMFGDLRSTFIALMIGSYASSAVTFPGIKLIYDAGVSFIVVLVVWAGCSGLVFLNCFFNWPLEPFPGPEDMDYSVKIKFSWLGFDHKITGKQFYKQVTTVGRRLSVGSSMRSAKEQVALQEGHKLCLSTVDLEVKCQPDAAVAPSFMHSVFSPILLLSLVTMCVTQLRLIFYMGAMNNILKFLVSGDQKTVGLYTSIFGVLQLLCLLTAPVIGYIMDWRLKECEDASEEPEEKDANQGEKKKKKRDRQIQKITNAMRAFAFTNLLLVGFGVTCLIPNLPLQILSFILHTIVRGFIHSAVGGLYAAVYPSTQFGSLTGLQSLISALFALLQQPLFLAMMGPLQGDPLWVNVGLLLLSLLGFCLPLYLICYRRQLERQLQQRQEDDKLFLKINGSSNQEAFV

SEQ ID NO: 12 - protein, 569 aa, Homo sapiens
MAPTLATAHRRRWWMACTAVLENLLFSAVLLGWGSLLIMLKSEGFYSYLCTEPENVTNGTVGGTAEPGHEEVSWMNGWLSCQAQDEMLNLAFTVGSFLLSAITLPLGIVMDKYGPRKLRLLGSACFAVSCLLIAYGASKPNALSVLIFIALALNGFGGMCMTFTSLTLPNMFGDLRSTFIALMIGSYASSAVTFPGIKLIYDAGVSFIVVLVVWAGCSGLVFLNCFFNWPLEPFPGPEDMDYSVKIKFSWLGFDHKITGKQFYKQVTTVGRRLSVGSSMRSAKEQVALQEGHKLCLSTVDLEVKCQPDAAVAPSFMHSVFSPILLLSLVTMCVTQLRLIFYMGAMNNILKFLVSGDQKTVGLYTSIFGVLQLLCLLTAPVIGYIMDWRLKECEDASEEPEEKDANQGEKKKKKRDRQIQKITNAMRAFAFTNLLLVGFGVTCLIPNLPLQILSFILHTIVRGFIHSAVGGLYAAVYPSTQFGSLTGLQSLISALFALLQQPLFLAMMGPLQGDPLWVNVGLLLLSLLGFCLPLYLICYRRQLERQLQQRQEDDKLFLKINGSSNQEAFV

SEQ ID NO: 13 - protein, 530 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMMLRLLSSLLLVAVASGYGPPSSRPSSRVVNGEDAVPYSWPWQVSLQYEKSGSFYHTCGGSLIAPDWVVTAGHCISSSWTYQVVLGEYDRAVKEGPEQVIPINSGDLFVHPLWNRSCVACGNDIALIKLSRSAQLGDAVQLASLPPAGDILPNETPCYITGWGRLYTNGPLPDKLQEALLPVVDYEHCSRWNWWGSSVKKTMVCAGGDIRSGCNGDSGGPLNCPTEDGGWQVHGVTSFVSAFGCNTRRKPTVFTRVSAFIDWIEETIASH

SEQ ID NO: 14 - protein, 270 aa, Homo sapiens [sequence omitted]

SEQ ID NO: 15 - protein, 579 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPLEQRSQHCKPEEGLQAQEEDLGLVGAQALQAEEQEAAFFSSTLNVGTLEELPAAESPSPPQSPQEESFSPTAMDAIFGSLSDEGSGSQEKEGPSTSPDLIDPESFSQDILHDKIIDLVHLLLRKYRVKGLITKAEMLGSVIKNYEDYFPEIFREASVCMQLLFGIDVKEVDPTSHSYVLVTSLNLSYDGIQCNEQSMPKSGLLIIVLGVIFMEGNCIPEEVMWEVLSIMGVYAGREHFLFGEPKRLLTQNWVQEKYLVYRQVPGTDPACYEFLWGPRAHAETSKMKVLEYIANANGRDPTSYPSLYEDALREEGEGV

SEQ ID NO: 16 - protein, 319 aa, Homo sapiens
MPLEQRSQHCKPEEGLQAQEEDLGLVGAQALQAEEQEAAFFSSTLNVGTLEELPAAESPSPPQSPQEESFSPTAMDAIFGSLSDEGSGSQEKEGPSTSPDLIDPESFSQDILHDKIIDLVHLLLRKYRVKGLITKAEMLGSVIKNYEDYFPEIFREASVCMQLLFGIDVKEVDPTSHSYVLVTSLNLSYDGIQCNEQSMPKSGLLIIVLGVIFMEGNCIPEEVMWEVLSIMGVYAGREHFLFGEPKRLLTQNWVQEKYLVYRQVPGTDPACYEFLWGPRAHAETSKMKVLEYIANANGRDPTSYPSLYEDALREEGEGV

SEQ ID NO: 17 - protein, 691 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSWVQATLLARGLCRAWGGTCGAALTGTSISQVPRRLPRGLHCSAAAHSSEQSLVPSPPEPRQRPTKALVPFEDLFGQAPGGERDKASFLQTVQKFAEHSVRKRGHIDFIYLALRKMREYGVERDLAVYNQLLNIFPKEVFRPRNIIQRIFVHYPRQQECGIAVLEQMENHGVMPNKETEFLLIQIFGRKSYPMLKLVRLKLWFPRFMNVNPFPVPRDLPQDPVELAMFGLRHMEPDLSARVTIYQVPLPKDSTGAADPPQPHIVGIQSPDQQAALARHNPARPVFVEGPFSLWLRNKCVYYHILRADLLPPEEREVEETPEEWNLYYPMQLDLEYVRSGWDNYEFDINEVEEGPVFAMCMAGAHDQATMAKWIQGLQETNPTLAQIPVVFRLAGSTRELQTSSAGLEEPPLPEDHQEEDDNLQRQQQGQS

SEQ ID NO: 18 - protein, 431 aa, Homo sapiens
MSWVQATLLARGLCRAWGGTCGAALTGTSISQVPRRLPRGLHCSAAAHSSEQSLVPSPPEPRQRPTKALVPFEDLFGQAPGGERDKASFLQTVQKFAEHSVRKRGHIDFIYLALRKMREYGVERDLAVYNQLLNIFPKEVFRPRNIIQRIFVHYPRQQECGIAVLEQMENHGVMPNKETEFLLIQIFGRKSYPMLKLVRLKLWFPRFMNVNPFPVPRDLPQDPVELAMFGLRHMEPDLSARVTIYQVPLPKDSTGAADPPQPHIVGIQSPDQQAALARHNPARPVFVEGPFSLWLRNKCVYYHILRADLLPPEEREVEETPEEWNLYYPMQLDLEYVRSGWDNYEFDINEVEEGPVFAMCMAGAHDQATMAKWIQGLQETNPTLAQIPVVFRLAGSTRELQTSSAGLEEPPLPEDHQEEDDNLQRQQQGQS

SEQ ID NO: 19 - protein, 681 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSAHSMLCERIAIAKELIKRAESLSRSRKGGIEGGAKLCSKLKAELKFLQKVEAGKVAIKESHLQSTNLTHLRAIVESAENLEEVVSVLHVFGYTDTLGEKQTLVVDVVANGGHTWVKAIGRKAEALHNIWLGRGQYGDKSIIEQAEDFLQASHQQPVQYSNPHIIFAFYNSVSSPMAEKLKEMGISVRGDIVAVNALLDHPEELQPSESESDDEGPELLQVTRVDRENILASVAFPTEIKVDVCKRVNLDITTLITYVSALSYGGCHFIFKEKVLTEQAEQERKEQVLPQLEAFMKDKELFACESAVKDFQSILDTLGGPGERERATVLIKRINVVPDQPSERALRLVASSKINSRSLTIFGTGDTLKAITMTANSGFVRAANNQGVKFSVFIHQPRALTESKEALATPLPKDYTTDSEH

SEQ ID NO: 20 - protein, 421 aa, Homo sapiens
MSAHSMLCERIAIAKELIKRAESLSRSRKGGIEGGAKLCSKLKAELKFLQKVEAGKVAIKESHLQSTNLTHLRAIVESAENLEEVVSVLHVFGYTDTLGEKQTLVVDVVANGGHTWVKAIGRKAEALHNIWLGRGQYGDKSIIEQAEDFLQASHQQPVQYSNPHIIFAFYNSVSSPMAEKLKEMGISVRGDIVAVNALLDHPEELQPSESESDDEGPELLQVTRVDRENILASVAFPTEIKVDVCKRVNLDITTLITYVSALSYGGCHFIFKEKVLTEQAEQERKEQVLPQLEAFMKDKELFACESAVKDFQSILDTLGGPGERERATVLIKRINVVPDQPSERALRLVASSKINSRSLTIFGTGDTLKAITMTANSGFVRAANNQGVKFSVFIHQPRALTESKEALATPLPKDYTTDSEH

SEQ ID NO: 21 - protein, 484 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMELYETSPYFYQEPRFYDGENYLPVHLQGFEPPGYERTELTLSPEAPGPLEDKGLGTPEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPGVPSECSSHSASCSPEWGSALEFSANPGDHLLTADPTDAHNLHSLTSIVDSITVEDVSVAFPDETMPN

SEQ ID NO: 22 - protein, 224 aa, Homo sapiens
MELYETSPYFYQEPRFYDGENYLPVHLQGFEPPGYERTELTLSPEAPGPLEDKGLGTPEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPGVPSECSSHSASCSPEWGSALEFSANPGDHLLTADPTDAHNLHSLTSIVDSITVEDVSVAFPDETMPN

SEQ ID NO: 23 - protein, 940 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMESSSSSNSYFSVGPTSPSAVVLLYSKELKKWDEFEDILEERRHVSDLKFAMKCYTPLVYKGITPCKPIDIKCSVLNSEEIHYVIKQLSKESLQSVDVLREEVSEILDEMSHKLRLGAIRFCAFTLSKVFKQIFSKVCVNEEGIQKLQRAIQEHPVVLLPSHRSYIDFLMLSFLLYNYDLPVPVIAAGMDFLGMKMVGELLRMSGAFFMRRTFGGNKLYWAVFSEYVKTMLRNGYAPVEFFLEGTRSRSAKTLTPKFGLLNIVMEPFFKREVFDTYLVPISISYDKILEETLYVYELLGVPKPKESTTGLLKARKILSENFGSIHVYFGDPVSLRSLAAGRMSRSSYNLVPRYIPQKQSEDMHAFVTEVAYKMELLQIENMVLSPWTLIVAVLLQNRPSMDFDALVEKTLWLKGLTQAFGGFLIWPDNKPAEEVVPASILLHSNIASLVKDQVILKVDSGDSEVVDGLMLQHITLLMCSAYRNQLLNIFVRPSLVAVALQMTPGFRKEDVYSCFRFLRDVFADEFIFLPGNTLKDFEEGCYLLCKSEAIQVTTKDILVTEKGNTVLEFLVGLFKPFVESYQIICKHLLSEEEDHFSEEQYLAAVRKFTSQLLDQGTSQCYDVLSSDVQKNALAACVRLGVVEKKKINNNCIFNVNEPATTKLEEMLGCKTPIGKPATAKL

SEQ ID NO: 24 - protein, 680 aa, Homo sapiens [sequence omitted]

SEQ ID NO: 25 - protein, 662 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 26 - protein, 402 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 27 - protein, 554 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSPRGTGCSAGLLMTVGWLLLAGLQSARGTNVTAAVQDAGLAHEGEGEEETENNDSETAENYAPPETEDVSNRNVVKEVEFGMCTVTCGIGVREVILTNGCPGGESKCVVRVEECRGPTDCGWGKPISESLESVRLACIHTSPLNRFKYMWKLLRQDQQSIILVNDSAILEVRKESHPLAFECDTLDNNEIVATIKFTVYTSSELQMRRSSLPATDAALIFVLTIGVIICVFIIFLLIFIIINWAAVKAFWGAKASTPEVQSEQSSVRYKDSTSLDQLPTEMPGEDDALSEWNE

SEQ ID NO: 28 - protein, 294 aa, Homo sapiens
MSPRGTGCSAGLLMTVGWLLLAGLQSARGTNVTAAVQDAGLAHEGEGEEETENNDSETAENYAPPETEDVSNRNVVKEVEFGMCTVTCGIGVREVILTNGCPGGESKCVVRVEECRGPTDCGWGKPISESLESVRLACIHTSPLNRFKYMWKLLRQDQQSIILVNDSAILEVRKESHPLAFECDTLDNNEIVATIKFTVYTSSELQMRRSSLPATDAALIFVLTIGVIICVFIIFLLIFIIINWAAVKAFWGAKASTPEVQSEQSSVRYKDSTSLDQLPTEMPGEDDALSEWNE

SEQ ID NO: 29 - protein, 535 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMRKRLSAPELRLSLTKGPGNDGASPTQSAPSSPDGSSDLEIDELETPSDSEQLDSGHEFEWEDELPRAEGLGTSETAERLGRGCMWDVTGEDGHHWRVFRMGPREQRVDMTVIEPYKKVLSHGGYHGDGLNAVILFASCYLPRSSIPNYTYVMEHLFRYMVGTLELLVAENYLLVHLSGGTSRAQVPPLSWIRQCYRTLDRRLRKNLRALVVVHATWYVKAFLALLRPFISSKFTRKIRFLDSLGELAQLISLDQVHIPEAVRQLDRDLHGSGGT

SEQ ID NO: 30 - protein, 275 aa, Homo sapiens
MRKRLSAPELRLSLTKGPGNDGASPTQSAPSSPDGSSDLEIDELETPSDSEQLDSGHEFEWEDELPRAEGLGTSETAERLGRGCMWDVTGEDGHHWRVFRMGPREQRVDMTVIEPYKKVLSHGGYHGDGLNAVILFASCYLPRSSIPNYTYVMEHLFRYMVGTLELLVAENYLLVHLSGGTSRAQVPPLSWIRQCYRTLDRRLRKNLRALVVVHATWYVKAFLALLRPFISSKFTRKIRFLDSLGELAQLISLDQVHIPEAVRQLDRDLHGSGGT

SEQ ID NO: 31 - protein, 734 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMELGGHWDMNSAPRLVSETAERKQEQKTGTEAEAADSGAVGARRFLLCLYLVGFLDLFGVSMVVPLLSLHVKSLGASPTVAGIVGSSYGILQLFSSTLVGCWSDVVGRRSSLLACILLSALGYLLLGAATNVFLFVLARVPAGIFKHTLSISRALLSDVVPEKERPLVIGHFNTASGVGFILGPVVGGYLTELEDGFYLTAFICFLVFILNAGLVWFFPWREAKPGSTEKGLPLRKTHVLLGRSHDTVQEAATSRRARASKKTAQPWVEVVLALRNMKNLLFSEMWDIFLVRLLMAMAVMLYYSNFVLALEERFGVRPKVTGYLISYSSMLGAVAGLALGPILRLYKHNSQALLLHSSILTCTLLLLYSLAPTMGAVVLSSTLLSFSTAIGRTCITDLQLTVGGAQASGTLIGVGQSVTAVGRIIAPLLSGVAQEVSPCGPPSLGAVLALVAIFIMSLNKRHSSGDGNSKLKSE

SEQ ID NO: 32 - protein, 474 aa, Homo sapiens
MELGGHWDMNSAPRLVSETAERKQEQKTGTEAEAADSGAVGARRFLLCLYLVGFLDLFGVSMVVPLLSLHVKSLGASPTVAGIVGSSYGILQLFSSTLVGCWSDVVGRRSSLLACILLSALGYLLLGAATNVFLFVLARVPAGIFKHTLSISRALLSDVVPEKERPLVIGHFNTASGVGFILGPVVGGYLTELEDGFYLTAFICFLVFILNAGLVWFFPWREAKPGSTEKGLPLRKTHVLLGRSHDTVQEAATSRRARASKKTAQPWVEVVLALRNMKNLLFSEMWDIFLVRLLMAMAVMLYYSNFVLALEERFGVRPKVTGYLISYSSMLGAVAGLALGPILRLYKHNSQALLLHSSILTCTLLLLYSLAPTMGAVVLSSTLLSFSTAIGRTCITDLQLTVGGAQASGTLIGVGQSVTAVGRIIAPLLSGVAQEVSPCGPPSLGAVLALVAIFIMSLNKRHSSGDGNSKLKSE

SEQ ID NO: 33 - protein, 656 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMESRCYGCAVKFTLFKKEYGCKNCGRAFCSGCLSFSAAVPRTGNTQQKVCKQCHEVLTRGSSANASKWSPPQNYKKRVAALEAKQKPSTSQSQGLTRQDQMIAERLARLRQENKPKLVPSQAEIEARLAALKDERQGSIPSTQEMEARLAALQGRVLPSQTPQPAHHTPDTRTQAQQTQDLLTQLAAEVAIDESWKGGGPAASLQNDLNQGGPGSTNSKRQANWSLEEEKSRLLAEAALELREENTRQERILALAKRLAMLRGQDPERVTLQDYRLPDSDDDEDEETAIQRVLQQLTEEAALDEASGFNIPAEQASRPWTQPRGAEPEAQDVDPRPEAEEEELPWCCICNEDATLRCAGCDGDLFCARCFREGHDAFELKEHQTSAYSPPRAGQEH

SEQ ID NO: 34 - protein, 396 aa, Homo sapiens
MESRCYGCAVKFTLFKKEYGCKNCGRAFCSGCLSFSAAVPRTGNTQQKVCKQCHEVLTRGSSANASKWSPPQNYKKRVAALEAKQKPSTSQSQGLTRQDQMIAERLARLRQENKPKLVPSQAEIEARLAALKDERQGSIPSTQEMEARLAALQGRVLPSQTPQPAHHTPDTRTQAQQTQDLLTQLAAEVAIDESWKGGGPAASLQNDLNQGGPGSTNSKRQANWSLEEEKSRLLAEAALELREENTRQERILALAKRLAMLRGQDPERVTLQDYRLPDSDDDEDEETAIQRVLQQLTEEAALDEASGFNIPAEQASRPWTQPRGAEPEAQDVDPRPEAEEEELPWCCICNEDATLRCAGCDGDLFCARCFREGHDAFELKEHQTSAYSPPRAGQEH

SEQ ID NO: 35 - protein, 581 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNRGGGSPSAAANYLLCTNCRKVLRKDKRIRVSQPLTRGPSAFIPEKEVVQANTVDERTNFLVEEYSTSGRLDNITQVMSLHTQYLESFLRSQFYMLRMDGPLPLPYRHYIAIMAAARHQCSYLINMHVDEFLKTGGIAEWLNGLEYVPQRLKNLNEINKLLAHRPWLITKEHIQKLVKTGENNWSLPELVHAVVLLAHYHALASFVFGSGINPERDPEISNGFRLISVNNFCVCDLANDNNIENASLSGSNFGIVDSLSELEALMERMKRLQEEREDEEASQEEMSTRFEKEKKESLFVVSGDTFHSFPHSGAFLHFFAF

SEQ ID NO: 36 - protein, 321 aa, Homo sapiens
MNRGGGSPSAAANYLLCTNCRKVLRKDKRIRVSQPLTRGPSAFIPEKEVVQANTVDERTNFLVEEYSTSGRLDNITQVMSLHTQYLESFLRSQFYMLRMDGPLPLPYRHYIAIMAAARHQCSYLINMHVDEFLKTGGIAEWLNGLEYVPQRLKNLNEINKLLAHRPWLITKEHIQKLVKTGENNWSLPELVHAVVLLAHYHALASFVFGSGINPERDPEISNGFRLISVNNFCVCDLANDNNIENASLSGSNFGIVDSLSELEALMERMKRLQEEREDEEASQEEMSTRFEKEKKESLFVVSGDTFHSFPHSGAFLHFFAF

SEQ ID NO: 37 - protein, 606 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMMDKFRMIFQFLQSNQESFMNGICGIMALASAQMYSAFDFNCPCLPGYNAAYSAGILLAPPLVLFLLGLVMNNNVSMLAEEWKRPPGRRAKDPAVLRYMFCSMAQRALIAPVVWVAVTLLDGKCFLCAFCTAVPVSALGNGSLAPGLPAPELARLLARVPCPEIYDGDWLLAREVAVRYLRCISQALGWSFVLLTTLLAFVVRSVRPCFTQAAFLKSKYWSHYIDIERKLFDETCTEHAKAFAKVCIQQFFEAMNHDLELGHTNGTLATAPASAAAPTTPDGAEEEREKLRGITDQGTMNRLLTSWHKCKPPLRLGQEEPPLMGNGWAGGGPRPPRKEVATYFSKV

SEQ ID NO: 38 - protein, 346 aa, Homo sapiens
MMDKFRMIFQFLQSNQESFMNGICGIMALASAQMYSAFDFNCPCLPGYNAAYSAGILLAPPLVLFLLGLVMNNNVSMLAEEWKRPPGRRAKDPAVLRYMFCSMAQRALIAPVVWVAVTLLDGKCFLCAFCTAVPVSALGNGSLAPGLPAPELARLLARVPCPEIYDGDWLLAREVAVRYLRCISQALGWSFVLLTTLLAFVVRSVRPCFTQAAFLKSKYWSHYIDIERKLFDETCTEHAKAFAKVCIQQFFEAMNHDLELGHTNGTLATAPASAAAPTTPDGAEEEREKLRGITDQGTMNRLLTSWHKCKPPLRLGQEEPPLMGNGWAGGGPRPPRKEVATYFSKV

SEQ ID NO: 39 - protein, 373 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSASVVSVISRFLEEYLSSIPQRLKLLDAYLLYILLTGALQFGYCLLVGTFPFNSFLSGFISCVGSFILAVCLRIQINPQNKADFQGISPERAFADFLFASTILHLVVMNFVG

SEQ ID NO: 40 - protein, 113 aa, Homo sapiens
MSASVVSVISRFLEEYLSSIPQRLKLLDAYLLYILLTGALQFGYCLLVGTFPFNSFLSGFISCVGSFILAVCLRIQINPQNKADFQGISPERAFADFLFASTILHLVVMNFVG

SEQ ID NO: 41 - protein, 729 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVISESMDILFRIRGGLDLAFQLATPNEIFLKKALKHVLSDLSTKLSSNALVFRICHSSVYIWPSSDINTIPGELTDASACKTILRFIQFEPEEDIKRKFMRKKDKKLSDMHQIVNIDLMLEMSTSLAAVTPIIERESGGHHYVNMTLPVDAVISVAPEETWGKVRKLLVDAIHNQLTDMEKCILKYMKGTSIVVPEPLHFLLPGKKNLVTISYPSGIPDGQLQAYRKELHDLFNLPHDRPYFKRSNAYHFPDEPYKDGYIRNPHTYLNPPNMETGMIYVVQGIYGYHHYMQDRIDDNGWGCAYRSLQTICSWFKHQGYTERSIPTHREIQQALVDAGDKPATFVGSRQWIGSIEVQLVLNQLIGITSKILFVSQGSEIASQGRELANHFQSEGTPVMIGGGVLAHTILGVAWNEITGQIKFLILDPHYTGAEDLQVILEKGWCGWKGPDFWNKDAYYNLCLPQRPNMI

SEQ ID NO: 42 - protein, 469 aa, Homo sapiens [sequence omitted]

SEQ ID NO: 43 - protein, 540 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAPRKGSSRVANTNSLRRRKLASFLKDFDREVEIRIKQIESDRQNLLKEVDNLYNIEILRLPKALREMNWLDYFALGGNKQALEEAATADLDITEINKLTAEAIQTPLKSAKTRKVIQVDEMIVEEEEEEENERKNLQTARVKRCPPSKKRTQSIQGKGKGKRSSRANTVTPAVGRLEVSMVKPTPGLTPRFDSRVFKTPGLRTPAAGERIYNISGNGSPLADSKEIFLTVPVGGGESLRLLASDLQRHSIAQLDPEALGNIKKLSNRLAQICSSIRTHK

SEQ ID NO: 44 - protein, 280 aa, Homo sapiens
MAPRKGSSRVANTNSLRRRKLASFLKDFDREVEIRIKQIESDRQNLLKEVDNLYNIEILRLPKALREMNWLDYFALGGNKQALEEAATADLDITEINKLTAEAIQTPLKSAKTRKVIQVDEMIVEEEEEEENERKNLQTARVKRCPPSKKRTQSIQGKGKGKRSSRANTVTPAVGRLEVSMVKPTPGLTPRFDSRVFKTPGLRTPAAGERIYNISGNGSPLADSKEIFLTVPVGGGESLRLLASDLQRHSIAQLDPEALGNIKKLSNRLAQICSSIRTHK

SEQ ID NO: 45 - protein, 486 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTVQRLVAAAVLVALVSLILNNVAAFTSNWVCQTLEDGRRRSVGLWRSCWLVDRTRGGPSPGARAGQVDAHDCEALGWGSEAAGFQESRGTVKLQFDMMRACNLVATAALTAGQLTFLLGLVGLPLLSPDAPCWEEAMAAAFQLASFVLVIGLVTFYRIGPYTNLSWSCYLNIGACLLATLAAAMLIWNILHKREDCMAPRVIVISRSLTARFRRGLDNDYVESPC

SEQ ID NO: 46 - protein, 226 aa, Homo sapiens
MTVQRLVAAAVLVALVSLILNNVAAFTSNWVCQTLEDGRRRSVGLWRSCWLVDRTRGGPSPGARAGQVDAHDCEALGWGSEAAGFQESRGTVKLQFDMMRACNLVATAALTAGQLTFLLGLVGLPLLSPDAPCWEEAMAAAFQLASFVLVIGLVTFYRIGPYTNLSWSCYLNIGACLLATLAAAMLIWNILHKREDCMAPRVIVISRSLTARFRRGLDNDYVESPC

SEQ ID NO: 47 - protein, 499 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 48 - protein, 239 aa, Homo sapiens
MAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 49 - protein, 577 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLLLGTVHCQIVSTRTPKPPLSTGGKRRRKLRKAAHLEVHREGDGSSTTDNTQEGAVQNHGTSTSHSVGTVFRDLWHAAFFLSGSKKAKNSIDKSTETDNGYVSLDGKKTVKSGEDGIQNHEPQCETIRPEETAWNTGTLRNGPSKDTQRTITNVSDEVSSEEGPETGYSLRRHVDRTSEGVLRNRKSHHYKKHYPNEDAPKSGTSCSSRCSSSRQDSESARPESETEDVLWEDLLHCAECHSSCTSETDVENHQINPCVKKEYRDDPFHQSHLPWLHSSHPGLEKISAIVWEGNDCKKADMSVLEISGMIMNRVSF

SEQ ID NO: 50 - protein, 317 aa, Homo sapiens
MLLLGTVHCQIVSTRTPKPPLSTGGKRRRKLRKAAHLEVHREGDGSSTTDNTQEGAVQNHGTSTSHSVGTVFRDLWHAAFFLSGSKKAKNSIDKSTETDNGYVSLDGKKTVKSGEDGIQNHEPQCETIRPEETAWNTGTLRNGPSKDTQRTITNVSDEVSSEEGPETGYSLRRHVDRTSEGVLRNRKSHHYKKHYPNEDAPKSGTSCSSRCSSSRQDSESARPESETEDVLWEDLLHCAECHSSCTSETDVENHQINPCVKKEYRDDPFHQSHLPWLHSSHPGLEKISAIVWEGNDCKKADMSVLEISGMIMNRVSF

SEQ ID NO: 51 - protein, 684 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGCGTSKVLPEPPKDVQLDLVKKVEPFSGTKSDVYKHFITEVDSVGPVKAGFPAASQYAHPCPGPPTAGHTEPPSEPPRRARVAKYRAKFDPRVTAKYDIKALIGRGSFSRVVRVEHRATRQPYAIKMIETKYREGREVCESELRVLRRVRHANIIQLVEVFETQERVYMVMELATGGELFDRIIAKGSFTERDATRVLQMVLDGVRYLHALGITHRDLKPENLLYYHPGTDSKIIITDFGLASARKKGDDCLMKTTCGTPEYIAPEVLVRKPYTNSVDMWALGVIAYILLSGTMPFEDDNRTRLYRQILRGKYSYSGEPWPSVSNLAKDFIDRLLTVDPGARMTALQALRHPWVVSMAASSSMKNLHRSISQNLLKRASSRCQSTKSAQSTRSSRSTRSNKSRRVRERELRELNLRYQQQYNG

SEQ ID NO: 52 - protein, 424 aa, Homo sapiens
MGCGTSKVLPEPPKDVQLDLVKKVEPFSGTKSDVYKHFITEVDSVGPVKAGFPAASQYAHPCPGPPTAGHTEPPSEPPRRARVAKYRAKFDPRVTAKYDIKALIGRGSFSRVVRVEHRATRQPYAIKMIETKYREGREVCESELRVLRRVRHANIIQLVEVFETQERVYMVMELATGGELFDRIIAKGSFTERDATRVLQMVLDGVRYLHALGITHRDLKPENLLYYHPGTDSKIIITDFGLASARKKGDDCLMKTTCGTPEYIAPEVLVRKPYTNSVDMWALGVIAYILLSGTMPFEDDNRTRLYRQILRGKYSYSGEPWPSVSNLAKDFIDRLLTVDPGARMTALQALRHPWVVSMAASSSMKNLHRSISQNLLKRASSRCQSTKSAQSTRSSRSTRSNKSRRVRERELRELNLRYQQQYNG

SEQ ID NO: 53 - protein, 463 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAQRKNAKSSGNSSSSGSGSGSTSAGSSSPGARRETKHGGHKNGRKGGLSGTSFFTWFMVIALLGVWTSVAVVWFDLVDYEEVLAKAKDFRYNLSEVLQGKLGIYDADGDGDFDVDDAKVLLGLTKDGSNENIDSLEEVLNILAEESSDWFYGFLSFLYDIMTPFEMLEEEEEESETADGVDGTSQNEGVQGKTCVILDLHNQ

SEQ ID NO: 54 - protein, 203 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLAKAKDFRYNLSEVLQGKLGIYDADGDGDFDVDDAKVLLGLTKDGSNENIDSLEEVLNILAEESSDWFYGFLSFLYDIMTPFEMLEEEEEESETADGVDGTSQNEGVQGKTCVILDLHNQ

SEQ ID NO: 55 - protein, 924 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEGFMDSGTQTDAVVVLSLAQAAVLGLVSENELFGATISAEAFYPDLGPELSGAAMGEPEPPGPDVYQLACNGRALEEPAEEEVLEVEAACEKHTRRKTRPPVRLVPKVKFEKVEEEEQEVYEVSVPGDDKDAGPAEAPAEAASGGCDALVQSSAVKMIDLSAFSRKPRTLRHLPRTPRPELNVAPYDPHFPAPARDGFPEPSMALPGPEALPTECGFEPPHLAPLSDPEAPSMESPEPVKPEQGFVWQEASEFEADTAGSTVERHKKAQLDRLDINVQIDDSYLVEAGDRQKRWQCRMCEKSYTSKYNLVTHILGHNGIKPHSCPHCSKLFKQPSHLQTHLLTHQGTRPHKCQVCHKAFTQTSHLKRHMLLHSEVKPYSCHFCGRGFAYPSELKAHEVKHESGRCHVCVECGLDFSTLTQLKRHLASHQGPTLYQCLECDKSFHYRSQLQNHMLKHQNVRPFVCTECGMEFSQIHHLKQHSLTHKGVKEFKCEVCGREFTLQANMKRHMLIHTSVRPYQCHICFKTFVQKQTLKTHMIVHSPVKPFKCKVCGKSFNRMYNLLGHMHLHAGSKPFKCPYCSSKFNLKGNLSRHMKVKHGVMDIGLDSQDPMMELTGTDPSELDGQQEMEDFEENAYSYASVDSSAEASVLTEQAMKEMAYYNVL

SEQ ID NO: 56 - protein, 664 aa, Homo sapiens
MEGFMDSGTQTDAVVVLSLAQAAVLGLVSENELFGATISAEAFYPDLGPELSGAAMGEPEPPGPDVYQLACNGRALEEPAEEEVLEVEAACEKHTRRKTRPPVRLVPKVKFEKVEEEEQEVYEVSVPGDDKDAGPAEAPAEAASGGCDALVQSSAVKMIDLSAFSRKPRTLRHLPRTPRPELNVAPYDPHFPAPARDGFPEPSMALPGPEALPTECGFEPPHLAPLSDPEAPSMESPEPVKPEQGFVWQEASEFEADTAGSTVERHKKAQLDRLDINVQIDDSYLVEAGDRQKRWQCRMCEKSYTSKYNLVTHILGHNGIKPHSCPHCSKLFKQPSHLQTHLLTHQGTRPHKCQVCHKAFTQTSHLKRHMLLHSEVKPYSCHFCGRGFAYPSELKAHEVKHESGRCHVCVECGLDFSTLTQLKRHLASHQGPTLYQCLECDKSFHYRSQLQNHMLKHQNVRPFVCTECGMEFSQIHHLKQHSLTHKGVKEFKCEVCGREFTLQANMKRHMLIHTSVRPYQCHICFKTFVQKQTLKTHMIVHSPVKPFKCKVCGKSFNRMYNLLGHMHLHAGSKPFKCPYCSSKFNLKGNLSRHMKVKHGVMDIGLDSQDPMMELTGTDPSELDGQQEMEDFEENAYSYASVDSSAEASVLTEQAMKEMAYYNVL

SEQ ID NO: 57 - protein, 1116 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASADKNGGSVSSVSSSRLQSRKPPNLSITIPPPEKETQAPGEQDSMLPEGFQNRRLKKSQPRTWASHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHALHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 58 - protein, 856 aa, Homo sapiens
MASADKNGGSVSSVSSSRLQSRKPPNLSITIPPPEKETQAPGEQDSMLPEGFQNRRLKKSQPRTWASHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHALHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 59 - protein, 468 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTAEKALPLGNGKAAEEARESEAPGGGCSSGGAAPARHPRVKRDKAVHERGHWNNKVEFVLSVAGEIIGLGNVWRFPYLCYKNGGGAFLIPYVVFFICCGIPVFFLETALGQFTSEGGITCWRKVCPLFEGIGYATQVIEAHLNVYYIIILAWAIFYLSNCFTTELPWATCGHEWNTENCVEFQKLNVSNYSHVSLQNATSPVMEFWE

SEQ ID NO: 60 - protein, 208 aa, Homo sapiens
MTAEKALPLGNGKAAEEARESEAPGGGCSSGGAAPARHPRVKRDKAVHERGHWNNKVEFVLSVAGEIIGLGNVWRFPYLCYKNGGGAFLIPYVVFFICCGIPVFFLETALGQFTSEGGITCWRKVCPLFEGIGYATQVIEAHLNVYYIIILAWAIFYLSNCFTTELPWATCGHEWNTENCVEFQKLNVSNYSHVSLQNATSPVMEFWE

SEQ ID NO: 61 - protein, 496 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 62 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 63 - protein, 661 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSSSGAKDKPELQFPFLQDEDTVATLLECKTLFILRGLPGSGKSTLARVIVDKYRDGTKMVSADAYKITPGARGAFSEEYKRLDEDLAAYCRRRDIRILVLDDTNHERERLEQLFEMADQYQYQVVLVEPKTAWRLDCAQLKEKNQWQLSADDLKKLKPGLEKDFLPLYFGWFLTKKSSETLRKAGQVFLEELGNHKAFKKELRQFVPGDEPREKMDLVTYFGKRPPGVLHCTTKFCDYGKAPGAEEYAQQDVLKKSYSKAFTLTISALFVTPKTTGARVELSEQQLQLWPSDVDKLSPTDNLPRGSRAHITLGCAADVEAVQTGLDLLEILRQEKGGSRGEEVGELSRGKLYSLGNGRWMLTLAKNMEVRAIFTGYYGKGKPVPTQGSRKGGALQSCTII

SEQ ID NO: 64 - protein, 401 aa, Homo sapiens
MSSSGAKDKPELQFPFLQDEDTVATLLECKTLFILRGLPGSGKSTLARVIVDKYRDGTKMVSADAYKITPGARGAFSEEYKRLDEDLAAYCRRRDIRILVLDDTNHERERLEQLFEMADQYQYQVVLVEPKTAWRLDCAQLKEKNQWQLSADDLKKLKPGLEKDFLPLYFGWFLTKKSSETLRKAGQVFLEELGNHKAFKKELRQFVPGDEPREKMDLVTYFGKRPPGVLHCTTKFCDYGKAPGAEEYAQQDVLKKSYSKAFTLTISALFVTPKTTGARVELSEQQLQLWPSDVDKLSPTDNLPRGSRAHITLGCAADVEAVQTGLDLLEILRQEKGGSRGEEVGELSRGKLYSLGNGRWMLTLAKNMEVRAIFTGYYGKGKPVPTQGSRKGGALQSCTII

SEQ ID NO: 65 - protein, 646 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAELEHLGGKRAESARMRRAEQLRRWRGSLTEQEPAERRGAGRQPLTRRGSPRVRFEDGAVFLAACSSGDTDEVRKLLARGADINTVNVDGLTALHQACIDENLDMVKFLVENRANVNQQDNEGWTPLHAAASCGYLNIAEYFINHGASVGIVNSEGEVPSDLAEEPAMKDLLLEQVKKQGVDLEQSRKEEEQQMLQDARQWLNSGKIEDVRQARSGATALHVAAAKGYSEVLRLLIQAGYELNVQDYDGWTPLHAAAHWGVKEACSILAEALCDMDIRNKLGQTPFDVADEGLVEHLELLQKKQNVLRSEKETRNKLIESDLNSKIQSGFFKNKEKMLYEEETPKSQEMEEENKESSSSSSEEEEGEDEASESETEKEAVLFWPF

SEQ ID NO: 66 - protein, 386 aa, Homo sapiens
MAELEHLGGKRAESARMRRAEQLRRWRGSLTEQEPAERRGAGRQPLTRRGSPRVRFEDGAVFLAACSSGDTDEVRKLLARGADINTVNVDGLTALHQACIDENLDMVKFLVENRANVNQQDNEGWTPLHAAASCGYLNIAEYFINHGASVGIVNSEGEVPSDLAEEPAMKDLLLEQVKKQGVDLEQSRKEEEQQMLQDARQWLNSGKIEDVRQARSGATALHVAAAKGYSEVLRLLIQAGYELNVQDYDGWTPLHAAAHWGVKEACSILAEALCDMDIRNKLGQTPFDVADEGLVEHLELLQKKQNVLRSEKETRNKLIESDLNSKIQSGFFKNKEKMLYEEETPKSQEMEEENKESSSSSSEEEEGEDEASESETEKEAVLFWPF

SEQ ID NO: 67 - protein, 1171 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 68 - protein, 911 aa, Homo sapiens
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 69 - protein, 775 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAYNGGTSAAATGHHHHHHHHLPHLPPPHLHHHHHPQHHLHPGSAAAVHPVQQHTSSAAAAAAAAAAAAAMLNPGQQQPYFPSPAPGQAPGPAAAAPAQVQAAAAATVKAHHHQHSHHPQQQLDIEPDRPIGYGAFGVVWSVTDPRDGKRVALKKMPNVFQNLVSCKRVFRELKMLCFFKHDNVLSALDILQPPHIDYFEEIYVVTELMQSDLHKIIVSPQPLSSDHVKVFLYQILRGLKYLHSAGILHRDIKPGNLLVNSNCVLKICDFGLARVEELDESRHMTQEVVTQYYRAPEILMGSRHYSNAIDIWSVGCIFAELLGRRILFQAQSPIQQLDLITDLLGTPSLEAMRTACEGAKAHILRGPHKQPSLPVLYTLSSQATHEAVHLLCRMLVFDPSKRISAKDALAHPYLDEGRLRYHTCMCKCCFSTSTGRVYTSDFEPVTNPKFDDTFEKNLSSVRQVKEIIHQFILEQQKGNRVPLCINPQSAAFKSFISSTVAQPSEMPPSPLVWE

SEQ ID NO: 70 - protein, 515 aa, Homo sapiens
MAAYNGGTSAAATGHHHHHHHHLPHLPPPHLHHHHHPQHHLHPGSAAAVHPVQQHTSSAAAAAAAAAAAAAMLNPGQQQPYFPSPAPGQAPGPAAAAPAQVQAAAAATVKAHHHQHSHHPQQQLDIEPDRPIGYGAFGVVWSVTDPRDGKRVALKKMPNVFQNLVSCKRVFRELKMLCFFKHDNVLSALDILQPPHIDYFEEIYVVTELMQSDLHKIIVSPQPLSSDHVKVFLYQILRGLKYLHSAGILHRDIKPGNLLVNSNCVLKICDFGLARVEELDESRHMTQEVVTQYYRAPEILMGSRHYSNAIDIWSVGCIFAELLGRRILFQAQSPIQQLDLITDLLGTPSLEAMRTACEGAKAHILRGPHKQPSLPVLYTLSSQATHEAVHLLCRMLVFDPSKRISAKDALAHPYLDEGRLRYHTCMCKCCFSTSTGRVYTSDFEPVTNPKFDDTFEKNLSSVRQVKEIIHQFILEQQKGNRVPLCINPQSAAFKSFISSTVAQPSEMPPSPLVWE

SEQ ID NO: 71 - protein, 786 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVAKDYPFYLTVKRANCSLELPPASGPAKDAEEPSNKRVKPLSRVTSLANLIPPVKATPLKRFSQTLQRSISFRSESRPDILAPRPWSRNAAPSSTKRRDSKLWSETFDVCVNQMLTSKEIKRQEAIFELSQGEEDLIEDLKLAKKAYHDPMLKLSIMTEQELNQIFGTLDSLIPLHEELLSQLRDVRKPDGSTEHVGPILVGWLPCLSSYDSYCSNQVAAKALLDHKKQDHRVQDFLQRCLESPFSRKLDLWNFLDIPRSRLVKYPLLLREILRHTPNDNPDQQHLEEAINIIQGIVAEINTKTGESECRYYKERLLYLEEGQKDSLIDSSRVLCCHGELKNNRGVKLHVFLFQEVLVITRAVTHNEQLCYQLYRQPIPVKDLLLEDLQDGEVRLGGSLRGAFSNNERIKNFFRVSFKNGSQSQTHSLQANDTFNKQQWLNCIRQAKETVLCAAGQAGVLDSEGSFLNPTTGSRELQGETKLEQMDQSDSESDCSMDTSEVSLDCERMEQTDSSCGNSRHGESNV

SEQ ID NO: 72 - protein, 526 aa, Homo sapiens
MVAKDYPFYLTVKRANCSLELPPASGPAKDAEEPSNKRVKPLSRVTSLANLIPPVKATPLKRFSQTLQRSISFRSESRPDILAPRPWSRNAAPSSTKRRDSKLWSETFDVCVNQMLTSKEIKRQEAIFELSQGEEDLIEDLKLAKKAYHDPMLKLSIMTEQELNQIFGTLDSLIPLHEELLSQLRDVRKPDGSTEHVGPILVGWLPCLSSYDSYCSNQVAAKALLDHKKQDHRVQDFLQRCLESPFSRKLDLWNFLDIPRSRLVKYPLLLREILRHTPNDNPDQQHLEEAINIIQGIVAEINTKTGESECRYYKERLLYLEEGQKDSLIDSSRVLCCHGELKNNRGVKLHVFLFQEVLVITRAVTHNEQLCYQLYRQPIPVKDLLLEDLQDGEVRLGGSLRGAFSNNERIKNFFRVSFKNGSQSQTHSLQANDTFNKQQWLNCIRQAKETVLCAAGQAGVLDSEGSFLNPTTGSRELQGETKLEQMDQSDSESDCSMDTSEVSLDCERMEQTDSSCGNSRHGESNV

SEQ ID NO: 73 - protein, 619 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMQAAALPEEIRWLLEDAEEFLAEGLRNENLSAVARDHRDHILRGFQQIKARYYWDFQPQGGDIGQDSSDDNHSGTLGLSLTSDAPFLSDYQDEGMEDIVKGAQELDNVIKQGYLEKKSKDHSFFGSEWQKRWCVVSRGLFYYYANEKSKQPKGTFLIKGYSVRMAPHLRRDSKKESCFELTSQDRRSYEFTATSPAEARDWVDQISFLLKDLSSLTIPYEEDEEEEEKEETYDDIDGFDSPSCGSQCRPTILPGSVGIKEPTEEKEEEDIYEVLPDEEHDLEEDESGTRRKGVDYASYYQGLWDCHGDQPDELSFQRGDLIRILSKEYNMYGWWVGELNSLVGIVPKEYLTTAFEVEER

SEQ ID NO: 74 - protein, 359 aa, Homo sapiens
MQAAALPEEIRWLLEDAEEFLAEGLRNENLSAVARDHRDHILRGFQQIKARYYWDFQPQGGDIGQDSSDDNHSGTLGLSLTSDAPFLSDYQDEGMEDIVKGAQELDNVIKQGYLEKKSKDHSFFGSEWQKRWCVVSRGLFYYYANEKSKQPKGTFLIKGYSVRMAPHLRRDSKKESCFELTSQDRRSYEFTATSPAEARDWVDQISFLLKDLSSLTIPYEEDEEEEEKEETYDDIDGFDSPSCGSQCRPTILPGSVGIKEPTEEKEEEDIYEVLPDEEHDLEEDESGTRRKGVDYASYYQGLWDCHGDQPDELSFQRGDLIRILSKEYNMYGWWVGELNSLVGIVPKEYLTTAFEVEER

SEQ ID NO: 75 - protein, 745 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGEPGFFVTGDRAGGRSWCLRRVGMSAGWLLLEDGCEVTVGRGFGVTYQLVSKICPLMISRNHCVLKQNPEGQWTIMDNKSLNGVWLNRARLEPLRVYSIHQGDYIQLGVPLENKENAEYEYEVTEEDWETIYPCLSPKNDQMIEKNKELRTKRKFSLDELAGPGAEGPSNLKSKINKVSCESGQPVKSQGKGEVASTPSDNLDPKLTALEPSKTTGAPIYPGFPKVTEVHHEQKASNSSASQRSLQMFKVTMSRILRLKIQMQEKHEAVMNVKKQTQKGNSKKVVQMEQELQDLQSQLCAEQAQQQARVEQLEKTFQEEEQHLQGLEIAQGEKDLKQQLAQALQEHWALMEELNRSKKDFEAIIQAKNKELEQTKEEKEKMQAQKEEVLSHMNDVLENELQCIICSEYFIEAVTLNCAHSFCSYCINEWMKRKIECPICRKDIKSKTYSLVLDNCINKMVNNLSSEVKERRIVLIRERKAKRLF

SEQ ID NO: 76 - protein, 485 aa, Homo sapiens
MGEPGFFVTGDRAGGRSWCLRRVGMSAGWLLLEDGCEVTVGRGFGVTYQLVSKICPLMISRNHCVLKQNPEGQWTIMDNKSLNGVWLNRARLEPLRVYSIHQGDYIQLGVPLENKENAEYEYEVTEEDWETIYPCLSPKNDQMIEKNKELRTKRKFSLDELAGPGAEGPSNLKSKINKVSCESGQPVKSQGKGEVASTPSDNLDPKLTALEPSKTTGAPIYPGFPKVTEVHHEQKASNSSASQRSLQMFKVTMSRILRLKIQMQEKHEAVMNVKKQTQKGNSKKVVQMEQELQDLQSQLCAEQAQQQARVEQLEKTFQEEEQHLQGLEIAQGEKDLKQQLAQALQEHWALMEELNRSKKDFEAIIQAKNKELEQTKEEKEKMQAQKEEVLSHMNDVLENELQCIICSEYFIEAVTLNCAHSFCSYCINEWMKRKIECPICRKDIKSKTYSLVLDNCINKMVNNLSSEVKERRIVLIRERKAKRLF

SEQ ID NO: 77 - protein, 719 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASGVEVLRFQLPGHEAATLRNMNQLRAEERFCDVTIVADSLKFRGHKVILAACSPFLRDQFLLNPSSELQVSLMHSARIVADLLLSCYTGALEFAVRDIVNYLTAASYLQMEHVVEKCRNALSQFIEPKIGLKEDGVSEASLVSSISATKSLLPPARTPKPAPKPPPPPPLPPPLLRPVKLEFPLDEDLELKAEEEDEDEDEDVSDICIVKVESALEVAHRLKPPGGLGGGLGIGGSVGGHLGELAQSSVPPSTVAPPQGVVKACYSLSEDAEGEGLLLIPGGRASVGATSGLVEAAAVAMAARGAGGSLGAGGSRGPLPGGFSGGNPLKNIKCTKCPEVFQGVEKLVFHMRAQHFIFMCPRCGKQFNHSSNLNRHMNVHRGVKSHSCGICGKCFTQKSTLHDHLNLHSGARPYRCSYCDVRFAHKPAIRRHLKEQHGKTTAENVLEASVAEINVLIR

SEQ ID NO: 78 - protein, 459 aa, Homo sapiens
MASGVEVLRFQLPGHEAATLRNMNQLRAEERFCDVTIVADSLKFRGHKVILAACSPFLRDQFLLNPSSELQVSLMHSARIVADLLLSCYTGALEFAVRDIVNYLTAASYLQMEHVVEKCRNALSQFIEPKIGLKEDGVSEASLVSSISATKSLLPPARTPKPAPKPPPPPPLPPPLLRPVKLEFPLDEDLELKAEEEDEDEDEDVSDICIVKVESALEVAHRLKPPGGLGGGLGIGGSVGGHLGELAQSSVPPSTVAPPQGVVKACYSLSEDAEGEGLLLIPGGRASVGATSGLVEAAAVAMAARGAGGSLGAGGSRGPLPGGFSGGNPLKNIKCTKCPEVFQGVEKLVFHMRAQHFIFMCPRCGKQFNHSSNLNRHMNVHRGVKSHSCGICGKCFTQKSTLHDHLNLHSGARPYRCSYCDVRFAHKPAIRRHLKEQHGKTTAENVLEASVAEINVLIR

SEQ ID NO: 79 - protein, 378 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTSQSQGIHQLLQAEKRAKDKLEEAKKRKGKRLKQAKEEAMVEIDQYRMQRDKEFRLKQSKIMGSQNNLSDEIEEQTLGKIQELNGHYNKYMESVMNQLLSMVCDMKPEIHVNYRATN

SEQ ID NO: 80 - protein, 118 aa, Homo sapiens
MTSQSQGIHQLLQAEKRAKDKLEEAKKRKGKRLKQAKEEAMVEIDQYRMQRDKEFRLKQSKIMGSQNNLSDEIEEQTLGKIQELNGHYNKYMESVMNQLLSMVCDMKPEIHVNYRATN

SEQ ID NO: 81 - protein, 377 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMIIDSSRIPSFTQLHSTMTRAPLLLLCVALVLLGHVNGATVRNEDKWKPLNNPRNRDLFFRRLQAYFKGRGLDLGTFPNPFPTNENPRPLSFQSELTASASADYEEQKNSFHNYLKG

SEQ ID NO: 82 - protein, 117 aa, Homo sapiens
MIIDSSRIPSFTQLHSTMTRAPLLLLCVALVLLGHVNGATVRNEDKWKPLNNPRNRDLFFRRLQAYFKGRGLDLGTFPNPFPTNENPRPLSFQSELTASASADYEEQKNSFHNYLKG

SEQ ID NO: 83 - protein, 504 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVHQDNCSYQAQKNERESIRQKLALGSFFDDGPGIYTSCSKSGKPSLSSRLQSGMNLQICFVNDSGSDKDSDADDSKTETSLDTPLSPMSKQSSSYSDRDTTEEESESLDDMDFLTRQKKLQAEAKMALAMAKPMAKMQVEVEKQNRKKSPVADLLPHMPHISECLMKRSLKPTDLRDMTIGQLQVIVNDLHSQIESLNEELVQLLLIRDELHTEQDAMLVDIEDLTRHAESQQKHMVEKMPAK

SEQ ID NO: 84 - protein, 244 aa, Homo sapiens
MVHQDNCSYQAQKNERESIRQKLALGSFFDDGPGIYTSCSKSGKPSLSSRLQSGMNLQICFVNDSGSDKDSDADDSKTETSLDTPLSPMSKQSSSYSDRDTTEEESESLDDMDFLTRQKKLQAEAKMALAMAKPMAKMQVEVEKQNRKKSPVADLLPHMPHISECLMKRSLKPTDLRDMTIGQLQVIVNDLHSQIESLNEELVQLLLIRDELHTEQDAMLVDIEDLTRHAESQQKHMVEKMPAK

SEQ ID NO: 85 - protein, 366 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMQKIVQTDEITNTQAFRKGKRKRTETMDSENANSDMDKGQRDPYSGNAFLPGESSSEDEEPLAELSKEELCAKIKSLKEKLTNTRKENSRLRQSLVMLQAWSRELP

SEQ ID NO: 86 - protein, 106 aa, Homo sapiens
MQKIVQTDEITNTQAFRKGKRKRTETMDSENANSDMDKGQRDPYSGNAFLPGESSSEDEEPLAELSKEELCAKIKSLKEKLTNTRKENSRLRQSLVMLQAWSRELP

SEQ ID NO: 87 - protein, 737 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKSLPILLLLCVAVCSAYPLDGAARGEDTSMNLVQKYLENYYDLEKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 88 - protein, 477 aa, Homo sapiens [sequence omitted]

SEQ ID NO: 89 - protein, 784 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSDNGELEDKPPAPPVRMSSTIFSTGGKDPLSANHSLKPLPSVPEEKKPRHKIISIFSGTEKGSKKKEKERPEISPPSDFEHTIHVGFDAVTGEFTGMPEQWARLLQTSNITKLEQKKNPQAVLDVLKFYDSNTVKQKYLSFTPPEKDGFPSGTPALNAKGTEAPAVVTEEEDDDEETAPPVIAPRPDHTKSIYTRSVIDPVPAPVGDSHVDGAAKSLDKQKKKTKMTDEEIMEKLRTIVSIGDPKKKYTRYEKIGQRASGTVFTATDVALGQEVAIKQINLQKQPKKELIINEILVMKELKNPNIVNFLDSYLVGDELFVVMEYLAGGSLTDVVTETCMDEAQIAAVCRECLQALEFLHANQVIHRDIKSDNVLLGMEGSVKLTDFGFCAQITPEQSKRSTMVGTPYWMAPEVVTRKAYGPKVDIWSLGIMAIEMVEGEPPYLNENPLRALYLIATNGTPELQNPEKLSPIFRDFLNRCLEMDVEKRGSAKELLQHPFLKLAKPLSSLTPLIMAAKEAMKSNR

SEQ ID NO: 90 - protein, 524 aa, Homo sapiens
MSDNGELEDKPPAPPVRMSSTIFSTGGKDPLSANHSLKPLPSVPEEKKPRHKIISIFSGTEKGSKKKEKERPEISPPSDFEHTIHVGFDAVTGEFTGMPEQWARLLQTSNITKLEQKKNPQAVLDVLKFYDSNTVKQKYLSFTPPEKDGFPSGTPALNAKGTEAPAVVTEEEDDDEETAPPVIAPRPDHTKSIYTRSVIDPVPAPVGDSHVDGAAKSLDKQKKKTKMTDEEIMEKLRTIVSIGDPKKKYTRYEKIGQRASGTVFTATDVALGQEVAIKQINLQKQPKKELIINEILVMKELKNPNIVNFLDSYLVGDELFVVMEYLAGGSLTDVVTETCMDEAQIAAVCRECLQALEFLHANQVIHRDIKSDNVLLGMEGSVKLTDFGFCAQITPEQSKRSTMVGTPYWMAPEVVTRKAYGPKVDIWSLGIMAIEMVEGEPPYLNENPLRALYLIATNGTPELQNPEKLSPIFRDFLNRCLEMDVEKRGSAKELLQHPFLKLAKPLSSLTPLIMAAKEAMKSNR

SEQ ID NO: 91 - protein, 1052 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMHVIKRDGRQERVMFDKITSRIQKLCYGLNMDFVDPAQITMKVIQGLYSGVTTVELDTLAAETAATLTTKHPDYAILAARIAVSNLHKETKKVFSDVMEDLYNYINPHNGKHSPMVAKSTLDIVLANKDRLNSAIIYDRDFSYNYFGFKTLERSYLLKINGKVAERPQHMLMRVSVGIHKEDIDAAIETYNLLSERWFTHASPTLFNAGTNRPQLSSCFLLSMKDDSIEGIYDTLKQCALISKSAGGIGVAVSCIRATGSYIAGTNGNSNGLVPMLRVYNNTARYVDQGGNKRPGAFAIYLEPWHLDIFEFLDLKKNTGKEEQRARDLFFALWIPDLFMKRVETNQDWSLMCPNECPGLDEVWGEEFEKLYASYEKQGRVRKVVKAQQLWYAIIESQTETGTPYMLYKDSCNRKSNQQNLGTIKCSNLCTEIVEYTSKDEVAVCNLASLALNMYVTSEHTYDFKKLAEVTKVVVRNLNKIIDINYYPVPEACLSNKRHRPIGIGVQGLADAFILMRYPFESAEAQLLNKQIFETIYYGALEASCDLAKEQGPYETYEGSPVSKGILQYDMWNVTPTDLWDWKVLKEKIAKYGIRNSLLIAPMPTASTAQILGNNESIEPYTSNIYTRRVLSGEFQIVNPHLLKDLTERGLWHEEMKNQIIACNGSIQSIPEIPDDLKQLYKTVWEISQKTVLKMAAERGAFIDQSQSLNIHIAEPNYGKLTSMHFYGWKQGLKTGMYYLRTRPAANPIQFTLNKEKLKDKEKVSKEEEEKERNTAAMVCSLENRDECLMCGS

SEQ ID NO: 92 - protein, 792 aa, Homo sapiens
MHVIKRDGRQERVMFDKITSRIQKLCYGLNMDFVDPAQITMKVIQGLYSGVTTVELDTLAAETAATLTTKHPDYAILAARIAVSNLHKETKKVFSDVMEDLYNYINPHNGKHSPMVAKSTLDIVLANKDRLNSAIIYDRDFSYNYFGFKTLERSYLLKINGKVAERPQHMLMRVSVGIHKEDIDAAIETYNLLSERWFTHASPTLFNAGTNRPQLSSCFLLSMKDDSIEGIYDTLKQCALISKSAGGIGVAVSCIRATGSYIAGTNGNSNGLVPMLRVYNNTARYVDQGGNKRPGAFAIYLEPWHLDIFEFLDLKKNTGKEEQRARDLFFALWIPDLFMKRVETNQDWSLMCPNECPGLDEVWGEEFEKLYASYEKQGRVRKVVKAQQLWYAIIESQTETGTPYMLYKDSCNRKSNQQNLGTIKCSNLCTEIVEYTSKDEVAVCNLASLALNMYVTSEHTYDFKKLAEVTKVVVRNLNKIIDINYYPVPEACLSNKRHRPIGIGVQGLADAFILMRYPFESAEAQLLNKQIFETIYYGALEASCDLAKEQGPYETYEGSPVSKGILQYDMWNVTPTDLWDWKVLKEKIAKYGIRNSLLIAPMPTASTAQILGNNESIEPYTSNIYTRRVLSGEFQIVNPHLLKDLTERGLWHEEMKNQIIACNGSIQSIPEIPDDLKQLYKTVWEISQKTVLKMAAERGAFIDQSQSLNIHIAEPNYGKLTSMHFYGWKQGLKTGMYYLRTRPAANPIQFTLNKEKLKDKEKVSKEEEEKERNTAAMVCSLENRDECLMCGS

SEQ ID NO: 93 - protein, 1050 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKAFGPPHEGPLQGLVASRIETYGGRHRASAQSTTGRLYPRGYPVLDPSRRRLQQYVPFARGSGQARGLSPMRLRDPEPEKRHGGHVGAGLLHSPKLKELTKAHELEVRLHTFSMFGMPRLPPEDRRHWEIGEGGDSGLTIEKSWRELVPGHKEMSQELCHQQEALWELLTTELIYVRKLKIMTDLLAAGLLNLQRVGLLMEVSAETLFGNVPSLIRTHRSFWDEVLGPTLEETRASGQPLDPIGLQSGFLTFGQRFHPYVQYCLRVKQTMAYAREQQETNPLFHAFVQWCEKHKRSGRQMLCDLLIKPHQRITKYPLLLHAVLKRSPEARAQEALNAMIEAVESFLRHINGQVRQGEEQESLAAAAQRIGPYEVLEPPSDEVGKNLRPFSTLDLTSPMLGVASEHTRQLLLEGPVRVKEGREGKLDVYLFLFSDVLLVTKPQRKADKAKVIRPPLMLEKLVCQPLRDPNSFLLIHLTEFQCVSSALLVHCPSPTDRAQWLEKTQQAQAALQKLKAEEYVQQKRELLTLYRDQDRESPSTRPSTPSLEGSQSSAEGRTPEFSTIIPHLVVTEDTDEDAPLVPDDTSDSGYGTLIPGTPTGSRSPLSRLRQRALRRDPRLTFSTLELRDIPLRPHPPDPQAPQRRSAPELPEGILKGGSLPQEDPPTWSEEEDGASERGNVVVETLHRARLRGQLPSSPTHADSAGESPWESSGEEEEEGPLFLKAGHTSLRPMRAEDMLREIREELASQRIEGAEEPRDSRPRKLTRAQLQRMRGPHIIQLDTPLSASEV

SEQ ID NO: 94 - protein, 790 aa, Homo sapiens [sequence omitted]

SEQ ID NO: 95 - protein, 654 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGMKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 96 - protein, 394 aa, Homo sapiens
MGMKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 97 - protein, 974 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNTLQGPVSFKDVAVDFTQEEWQQLDPDEKITYRDVMLENYSHLVSVGYDTTKPNVIIKLEQGEEPWIMGGEFPCQHSPEAWRVDDLIERIQENEDKHSRQAACINSKTLTEEKENTFSQIYMETSLVPSSIIAHNCVSCGKNLESISQLISSDGSYARTKPDECNECGKTYHGEKMCEFNQNGDTYSHNEENILQKISILEKPFEYNECMEALDNEAVFIAHKRAYIGEKPYEWNDSGPDFIQMSNFNAYQRSQMEMKPFECSECGKSFCKKSKFIIHQRAHTGEKPYECNVCGKSFSQKGTLTVHRRSHLEEKPYKCNECGKTFCQKLHLTQHLRTHSGEKPYECSECGKTFCQKTHLTLHQRNHSGERPYPCNECGKSFSRKSALSDHQRTHTGEKLYKCNECGKSYYRKSTLITHQRTHTGEKPYQCSECGKFFSRVSYLTIHYRSHLEEKPYECNECGKTFNLNSAFIRHRKVHTEEKSHECSECGKFSQLYLTDHHTAHLEEKPYECNECGKTFLVNSAFDGHQPLPKGEKSYECNVCGKLFNELSYYTEHYRSHSEEKPYGCSECGKTFSHNSSLFRHQRVHTGEKPYECYECGKFFSQKSYLTIHHRIHSGEKPYECSKCGKVFSRMSNLTVHYRSHSGEKPYECNECGKVFSQKSYLTVHYRTHSGEKPYECNECGKKFHHRSAFNSHQRIHRRGNMNVLDVENL

SEQ ID NO: 98 - protein, 714 aa, Homo sapiens
MNTLQGPVSFKDVAVDFTQEEWQQLDPDEKITYRDVMLENYSHLVSVGYDTTKPNVIIKLEQGEEPWIMGGEFPCQHSPEAWRVDDLIERIQENEDKHSRQAACINSKTLTEEKENTFSQIYMETSLVPSSIIAHNCVSCGKNLESISQLISSDGSYARTKPDECNECGKTYHGEKMCEFNQNGDTYSHNEENILQKISILEKPFEYNECMEALDNEAVFIAHKRAYIGEKPYEWNDSGPDFIQMSNFNAYQRSQMEMKPFECSECGKSFCKKSKFIIHQRAHTGEKPYECNVCGKSFSQKGTLTVHRRSHLEEKPYKCNECGKTFCQKLHLTQHLRTHSGEKPYECSECGKTFCQKTHLTLHQRNHSGERPYPCNECGKSFSRKSALSDHQRTHTGEKLYKCNECGKSYYRKSTLITHQRTHTGEKPYQCSECGKFFSRVSYLTIHYRSHLEEKPYECNECGKTFNLNSAFIRHRKVHTEEKSHECSECGKFSQLYLTDHHTAHLEEKPYECNECGKTFLVNSAFDGHQPLPKGEKSYECNVCGKLFNELSYYTEHYRSHSEEKPYGCSECGKTFSHNSSLFRHQRVHTGEKPYECYECGKFFSQKSYLTIHHRIHSGEKPYECSKCGKVFSRMSNLTVHYRSHSGEKPYECNECGKVFSQKSYLTVHYRTHSGEKPYECNECGKKFHHRSAFNSHQRIHRRGNMNVLDVENL

SEQ ID NO: 99 - protein, 803 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSLKWTSVFLLIQLSCYFSSGSCGKVLVWPTEYSHWINMKTILEELVQRGHEVTVLTSSASTLVNASKSSAIKLEVYPTSLTKNYLEDSLLKILDRWIYGVSKNTFWSYFSQLQELCWEYYDYSNKLCKDAVLNKKLMMKLQESKFDVILADALNPCGELLAELFNIPFLYSLRFSVGYTFEKNGGGFLFPPSYVPVVMSELSDQMIFMERIKNMIHMLYFDFWFQIYDLKKWDQFYSEVLGRPTTLFETMGKAEMWLIRTYWDFEFPRPFLPNVDFVGGLHCKPAKPLPKEMEEFVQSSGENGIVVFSLGSMISNMSEESANMIASALAQIPQKVLWRFDGKKPNTLGSNTRLYKWLPQNDLLGHPKTKAFITHGGTNGIYEAIYHGIPMVGIPLFADQHDNIAHMKAKGAALSVDIRTMSSRDLLNALKSVINDPVYKENVMKLSRIHHDQPMKPLDRAVFWIEFVMRHKGAKHLRVAAHNLTWIQYHSLDVIAFLLACVATVIFIITKFCLFCFRKLAKKGKKKKRDNPAFLYKVVKASS

SEQ ID NO: 100 - protein, 530 aa, Homo sapiens
MSLKWTSVFLLIQLSCYFSSGSCGKVLVWPTEYSHWINMKTILEELVQRGHEVTVLTSSASTLVNASKSSAIKLEVYPTSLTKNYLEDSLLKILDRWIYGVSKNTFWSYFSQLQELCWEYYDYSNKLCKDAVLNKKLMMKLQESKFDVILADALNPCGELLAELFNIPFLYSLRFSVGYTFEKNGGGFLFPPSYVPVVMSELSDQMIFMERIKNMIHMLYFDFWFQIYDLKKWDQFYSEVLGRPTTLFETMGKAEMWLIRTYWDFEFPRPFLPNVDFVGGLHCKPAKPLPKEMEEFVQSSGENGIVVFSLGSMISNMSEESANMIASALAQIPQKVLWRFDGKKPNTLGSNTRLYKWLPQNDLLGHPKTKAFITHGGTNGIYEAIYHGIPMVGIPLFADQHDNIAHMKAKGAALSVDIRTMSSRDLLNALKSVINDPVYKENVMKLSRIHHDQPMKPLDRAVFWIEFVMRHKGAKHLRVAAHNLTWIQYHSLDVIAFLLACVATVIFIITKFCLFCFRKLAKKGKKKKRD

SEQ ID NO: 101 - protein, 519 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGINPAFLYKVVKASS

SEQ ID NO: 102 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 103 - protein, 1296 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMFHLRTCAAKLRPLTASQTVKTFSQNRPAAARTFQQIRCYSAPVAAEPFLSGTSSNYVEEMYCAWLENPKSVHKSWDIFFRNTNAGAPPGTAYQSPLPLSRGSLAAVAHAQSLVEAQPNVDKLVEDHLAVQSLIRAYQIRGHHVAQLDPLGILDADLDSSVPADIISSTDKLGFYGLDESDLDKVFHLPTTTFIGGQESALPLREIIRRLEMAYCQHIGVEFMFINDLEQCQWIRQKFETPGIMQFTNEEKRTLLARLVRSTRFEEFLQRKWSSEKRFGLEGCEVLIPALKTIIDKSSENGVDYVIMGMPHRGRLNVLANVIRKELEQIFCQFDSKLEAADEGSGDVKYHLGMYHRRINRVTDRNITLSLVANPSHLEAADPVVMGKTKAEQFYCGDTEGKKVMSILLHGDAAFAGQGIVYETFHLSDLPSYTTHGTVHVVVNNQIGFTTDPRMARSSPYPTDVARVVNAPIFHVNSDDPEAVMYVCKVAAEWRSTFHKDVVVDLVCYRRNGHNEMDEPMFTQPLMYKQIRKQKPVLQKYAELLVSQGVVNQPEYEEEISKYDKICEEAFARSKDEKILHIKHWLDSPWPGFFTLDGQPRSMSCPSTGLTEDILTHIGNVASSVPVENFTIHGGLSRILKTRGEMVKNRTVDWALAEYMAFGSLLKEGIHIRLSGQDVERGTFSHRHHVLHDQNVDKRTCIPMNHLWPNQAPYTVCNSSLSEYGVLGFELGFAMASPNALVLWEAQFGDFHNTAQCIIDQFICPGQAKWVRQNGIVLLLPHGMEGMGPEHSSARPERFLQMCNDDPDVLPDLKEANFDINQLYDCNWVVVNCSTPGNFFHVLRRQILLPFRKPLIIFTPKSLLRHPEARSSFDEMLPGTHFQRVIPEDGPAAQNPENVKRLLFCTGKVYYDLTRERKARDMVGQVAITRIEQLSPFPFDLLLKEVQKYPNAELAWCQEEHKNQGYYDYVKPRLRTTISRAKPVWYAGRDPAAAPATGNKKTHLTELQRLLDTAFDLDVFKNFSNPAFLYKVVKASS

SEQ ID NO: 104 - protein, 1023 aa, Homo sapiens
MFHLRTCAAKLRPLTASQTVKTFSQNRPAAARTFQQIRCYSAPVAAEPFLSGTSSNYVEEMYCAWLENPKSVHKSWDIFFRNTNAGAPPGTAYQSPLPLSRGSLAAVAHAQSLVEAQPNVDKLVEDHLAVQSLIRAYQIRGHHVAQLDPLGILDADLDSSVPADIISSTDKLGFYGLDESDLDKVFHLPTTTFIGGQESALPLREIIRRLEMAYCQHIGVEFMFINDLEQCQWIRQKFETPGIMQFTNEEKRTLLARLVRSTRFEEFLQRKWSSEKRFGLEGCEVLIPALKTIIDKSSENGVDYVIMGMPHRGRLNVLANVIRKELEQIFCQFDSKLEAADEGSGDVKYHLGMYHRRINRVTDRNITLSLVANPSHLEAADPVVMGKTKAEQFYCGDTEGKKVMSILLHGDAAFAGQGIVYETFHLSDLPSYTTHGTVHVVVNNQIGFTTDPRMARSSPYPTDVARVVNAPIFHVNSDDPEAVMYVCKVAAEWRSTFHKDVVVDLVCYRRNGHNEMDEPMFTQPLMYKQIRKQKPVLQKYAELLVSQGVVNQPEYEEEISKYDKICEEAFARSKDEKILHIKHWLDSPWPGFFTLDGQPRSMSCPSTGLTEDILTHIGNVASSVPVENFTIHGGLSRILKTRGEMVKNRTVDWALAEYMAFGSLLKEGIHIRLSGQDVERGTFSHRHHVLHDQNVDKRTCIPMNHLWPNQAPYTVCNSSLSEYGVLGFELGFAMASPNALVLWEAQFGDFHNTAQCIIDQFICPGQAKWVRQNGIVLLLPHGMEGMGPEHSSARPERFLQMCNDDPDVLPDLKEANFDINQLYDCNWVVVNCSTPGNFFHVLRRQILLPFRKPLIIFTPKSLLRHPEARSSFDEMLPGTHFQRVIPEDGPAAQNPENVKRLLFCTGKVYYDLTRERKARDMVGQVAITRIEQLSPFPFDLLLKEVQKYPNAELAWCQEEHKNQGYYDYVKPRLRTTISRAKPVWYAGRDPAAAPATGNKKTHLTELQRLLDTAFDLDVFKNFS

SEQ ID NO: 105 - protein, 696 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEDKRSLSMARCEERNSRGQDHGLERVPFPPQLQSETYLHPADPSPAWDDPGSTGSPNLRLLTEEIAFQPLAEEASFRRPHPDGDVPPQGEDNLLSLPFPQKLWRLVSSNQFSSIWWDDSGACRVINQKLFEKEILKRDVAHKVFATTSIKSFFRQLNLYGFRKRRQCTFRTFTRIFSAKRLVSILNKLEFYCHPYFQRDSPHLLVRMKRRVGVKSAPRHQEEDKPEAAGSCLAPADTEQQDHTSPNENDQVTPQHREPAGPNTQIRSGSAPPATPVMVPDSAVASDNSPVTQPAGEWSEGSQAHVTPVAAVPGPAALPFLYVPGSPTQMNSYGPVVALPTASRSTLAMDTTGLPAPGMLPFCHLWVPVTLVAAGAAQPAASMVMFPHLPALHHHCPHSHRTSQYMPASDGPQAYPDYADQSTNPAFLYKVVKASS

SEQ ID NO: 106 - protein, 423 aa, Homo sapiens
MEDKRSLSMARCEERNSRGQDHGLERVPFPPQLQSETYLHPADPSPAWDDPGSTGSPNLRLLTEEIAFQPLAEEASFRRPHPDGDVPPQGEDNLLSLPFPQKLWRLVSSNQFSSIWWDDSGACRVINQKLFEKEILKRDVAHKVFATTSIKSFFRQLNLYGFRKRRQCTFRTFTRIFSAKRLVSILNKLEFYCHPYFQRDSPHLLVRMKRRVGVKSAPRHQEEDKPEAAGSCLAPADTEQQDHTSPNENDQVTPQHREPAGPNTQIRSGSAPPATPVMVPDSAVASDNSPVTQPAGEWSEGSQAHVTPVAAVPGPAALPFLYVPGSPTQMNSYGPVVALPTASRSTLAMDTTGLPAPGMLPFCHLWVPVTLVAAGAAQPAASMVMFPHLPALHHHCPHSHRTSQYMPASDGPQAYPDYADQST

SEQ ID NO: 107 - protein, 806 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMMSEGKPPDKKRPRRSLSISKNKKKASNSIISCFNNAPPAKLACPVCSKMVPRYDLNRHLDEMCANNDFVQVDPGQVGLINSNVSMVDLTSVTLEDVTPKKSPPPKTNLTPGQSDSAKREVKQKISPYFKSNDVVCKNQDELRNRSVKVICLGSLASKLSRKYVKAKKSIDKDEEFAGSSPQSSKSTVVKSLIDNSSEIEDEDQILENSSQKENVFKCDSLKEECIPEHMVRGSKIMEAESQKATRECEKSALTPGFSDNAIMLFSPDFTLRNTLKSTSEDSLVKQECIKEVVEKREACHCEEVKMTVASEAKIQLSDSEAKSHSSADDASAWSNIQEAPLQDDSCLNNDIPHSIPLEQGSSCNGPGQTTGHPYYLRSFLVVLKTVLENEDDMLLFDEQEKGIVTKFYQLSATGQKLYVRLFQRKLSWIKMTKLEYEEIALDLTPVIEELTNAGFLQTESELQELSEVLELLSAPELKSLAKTFHLVNPNGQKQQLVDAFLKLAKQRSVCTWGKNKPGIGAVILKRFCWLLLQNPAFLYKVVKASS

SEQ ID NO: 108 - protein, 533 aa, Homo sapiens
MMSEGKPPDKKRPRRSLSISKNKKKASNSIISCFNNAPPAKLACPVCSKMVPRYDLNRHLDEMCANNDFVQVDPGQVGLINSNVSMVDLTSVTLEDVTPKKSPPPKTNLTPGQSDSAKREVKQKISPYFKSNDVVCKNQDELRNRSVKVICLGSLASKLSRKYVKAKKSIDKDEEFAGSSPQSSKSTVVKSLIDNSSEIEDEDQILENSSQKENVFKCDSLKEECIPEHMVRGSKIMEAESQKATRECEKSALTPGFSDNAIMLFSPDFTLRNTLKSTSEDSLVKQECIKEVVEKREACHCEEVKMTVASEAKIQLSDSEAKSHSSADDASAWSNIQEAPLQDDSCLNNDIPHSIPLEQGSSCNGPGQTTGHPYYLRSFLVVLKTVLENEDDMLLFDEQEKGIVTKFYQLSATGQKLYVRLFQRKLSWIKMTKLEYEEIALDLTPVIEELTNAGFLQTESELQELSEVLELLSAPELKSLAKTFHLVNPNGQKQQLVDAFLKLAKQRSVCTWGKNKPGIGAVILKRFCWLLLQ

SEQ ID NO: 109 - protein, 818 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNHNPAFLYKVVKASS

SEQ ID NO: 110 - protein, 545 aa, Homo sapiens
MSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNH

SEQ ID NO: 111 - protein, 577 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKSDNHSFLGDSPKAFILLGVSDRPWLELPLFVVLLLSYVLAMLGNVAIILASRVDPQLHSPMYIFLSHLSFLDLCYTTTTVPQMLVNMGSSQKTISYGGCTVQYAVFHWLGCTECIVLAAMALDRYVAICKPLHYAVLMHRALCQQLVALAWLSGFGNSFVQVVLTVQLPFCGRQVLNNFFCEVPAVIKLSCADTAMNDTILAVLVAFFVLVPLALILLSYGFIARAVLRIQSSKGRHKAFGTCSSHLMIVSLFYLPAIYMYLQPPSSYSQEQGKFISLFYSIITPTLNPFTYTLRNKDMKGALRRLLARIWRLCG

SEQ ID NO: 112 - protein, 317 aa, Homo sapiens [sequence omitted]

SEQ ID NO: 113 - protein, 484 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTLFHFGNCFALAYFPYFITYKCSGLSEYNAFWKCVQAGVTYLFVQLCKMLFLATFFPTWEGGIYDFIGEFMKASVDVADLIGLNLVMSRNAGKGEYKIMVAALGWATAELIMSRCIPLWVGARGIEFDWKYIQMSIDSNISLVHYIVASAQVWMITRYDLYHTFRPAVLLLMFLSVYKAFVMETFVHLCSLGSWAALLARAVVTGLLALSTLALYVAVVNVHS

SEQ ID NO: 114 - protein, 224 aa, Homo sapiens
MTLFHFGNCFALAYFPYFITYKCSGLSEYNAFWKCVQAGVTYLFVQLCKMLFLATFFPTWEGGIYDFIGEFMKASVDVADLIGLNLVMSRNAGKGEYKIMVAALGWATAELIMSRCIPLWVGARGIEFDWKYIQMSIDSNISLVHYIVASAQVWMITRYDLYHTFRPAVLLLMFLSVYKAFVMETFVHLCSLGSWAALLARAVVTGLLALSTLALYVAVVNVHS

SEQ ID NO: 115 - protein, 407 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGFIFSKSMNESMKNQKEFMLMNARLQLERQLIMQSEMRERQMAMQIAWSREFLKYFGTFFGLAAISLTAGAIKKKKPAFLVPIVPLSFILTYQYDLGYGTLLERMKGEAEDILETEKSKLQLPRGMITFESIEKARKEQSRFFIDK

SEQ ID NO: 116 - protein, 147 aa, Homo sapiens
MGFIFSKSMNESMKNQKEFMLMNARLQLERQLIMQSEMRERQMAMQIAWSREFLKYFGTFFGLAAISLTAGAIKKKKPAFLVPIVPLSFILTYQYDLGYGTLLERMKGEAEDILETEKSKLQLPRGMITFESIEKARKEQSRFFIDK

SEQ ID NO: 117 - protein, 417 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSDFDSNPFADPDLNNPFKDPSVTQVTRNVPPGLDEYNPFSDSRTPPPGGVKMPNVPNTQPAIMKPTEEHPAYTQIAKEHALAQAELLKRQEELERKAAELDRREREMQNLSQHGRKNNWPPLPSNFPVGPCFYQDFSVDIPVEFQKTVKLMYYLWM

SEQ ID NO: 118 - protein, 157 aa, Homo sapiens
MSDFDSNPFADPDLNNPFKDPSVTQVTRNVPPGLDEYNPFSDSRTPPPGGVKMPNVPNTQPAIMKPTEEHPAYTQIAKEHALAQAELLKRQEELERKAAELDRREREMQNLSQHGRKNNWPPLPSNFPVGPCFYQDFSVDIPVEFQKTVKLMYYLWM

SEQ ID NO: 119 - protein, 617 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEVLRPQLIRIDGRNYRKNPVQEQTYQHEEDEEDFYQGSMECADEPCDAYEVEQTPQGFRSTLRAPSLLYKHIVGKRGDTRKKIEMETKTSISIPKPGQDGEIVITGQHRNGVISARTRIDVLLDTFRRKQPFTHFLAFFLNEVEVQEGFLRFQEEVLAKCSMDHGVDSSIFQNPKKLHLTIGMLVLLSEEEIQQTCEMLQQCKEEFINDISGGKPLEVEMAGIEYMNDDPGMVDVLYAKVHMKDGSNRLQELVDRVLERFQASGLIVKEWNSVKLHATVMNTLFRKDPNAEGRYNLYTAEGKYIFKERESFDGRNILKLFENFYFGSLKLNSIHISQRFTVDSFGNYASCGQIDFS

SEQ ID NO: 120 - protein, 357 aa, Homo sapiens
MEVLRPQLIRIDGRNYRKNPVQEQTYQHEEDEEDFYQGSMECADEPCDAYEVEQTPQGFRSTLRAPSLLYKHIVGKRGDTRKKIEMETKTSISIPKPGQDGEIVITGQHRNGVISARTRIDVLLDTFRRKQPFTHFLAFFLNEVEVQEGFLRFQEEVLAKCSMDHGVDSSIFQNPKKLHLTIGMLVLLSEEEIQQTCEMLQQCKEEFINDISGGKPLEVEMAGIEYMNDDPGMVDVLYAKVHMKDGSNRLQELVDRVLERFQASGLIVKEWNSVKLHATVMNTLFRKDPNAEGRYNLYTAEGKYIFKERESFDGRNILKLFENFYFGSLKLNSIHISQRFTVDSFGNYASCGQIDFS

SEQ ID NO: 121 - protein, 571 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQESENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 122 - protein, 311 aa, Homo sapiens [sequence omitted]

SEQ ID NO: 123 - protein, 528 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAVRALVASRLAAASAFTSLSPGGRTPSQRAALHLSVPRPAARVALVLSGCGVYDGTEIHEASAILVHLSRGGAEVQIFAPDVPQMHVIDHTKGQPSEGESRNVLTESARIARGKITDLANLSAANHDAAIFPGGFGAAKNLSTFAVDGKDCKVNKEVERVLKEFHQAGKPIGLCCIAPVLAAKVLRGVEVTVGHEQEEGGKWPYAGTAEAIKALGAKHCVKEVVEAHVDQKNKVVTTPAFMCETALHYIHDGIGAMVRKVLELTGK

SEQ ID NO: 124 - protein, 268 aa, Homo sapiens
MAAVRALVASRLAAASAFTSLSPGGRTPSQRAALHLSVPRPAARVALVLSGCGVYDGTEIHEASAILVHLSRGGAEVQIFAPDVPQMHVIDHTKGQPSEGESRNVLTESARIARGKITDLANLSAANHDAAIFPGGFGAAKNLSTFAVDGKDCKVNKEVERVLKEFHQAGKPIGLCCIAPVLAAKVLRGVEVTVGHEQEEGGKWPYAGTAEAIKALGAKHCVKEVVEAHVDQKNKVVTTPAFMCETALHYIHDGIGAMVRKVLELTGK

SEQ ID NO: 125 - protein, 446 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVRPKKVCFSESSLPTGDRTRRSYYLNEIQSFAGAEKDARVVGEIAFQLDRRILAYVFPGVTRLYGFTVANIPEKIEQTSTKSLDGSVDERKLRELTQRYLALSARLEKLGYSRDVHPAFSEFLINTYGILKQRPDLRANPLHSSPAALRKLVIDVVPPKFLGDSLLLLNCLCELSKEDGKPLFAW

SEQ ID NO: 126 - protein, 186 aa, Homo sapiens
MVRPKKVCFSESSLPTGDRTRRSYYLNEIQSFAGAEKDARVVGEIAFQLDRRILAYVFPGVTRLYGFTVANIPEKIEQTSTKSLDGSVDERKLRELTQRYLALSARLEKLGYSRDVHPAFSEFLINTYGILKQRPDLRANPLHSSPAALRKLVIDVVPPKFLGDSLLLLNCLCELSKEDGKPLFAW

SEQ ID NO: 127 - protein, 543 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTSGPGGPAAAAGGRKENHQWYVCNREKLCESLQAVFVQSYLDQGTQIFLNNSIEKSGWLFIQLYHSFVSSVFSLFMSRTSINGLLGRGSMFVFSPDQFQRLLKINPDWKTHRLLDLGAGDGEVTKIMSPHFEEIYATELSETMIWQLQKKKYRVLGINEWQNTGFQYDVISCLNLLDRCDQPLTLLKDIRSVLEPTRGRVILALVLPFHPYVENVGGKWEKPSEILEIKGQNWEEQVNSLPEVFRKAGFVIEAFTRLPYLCEGDMYNDYYVLDDAVFVLKPV

SEQ ID NO: 128 - protein, 283 aa, Homo sapiens
MTSGPGGPAAAAGGRKENHQWYVCNREKLCESLQAVFVQSYLDQGTQIFLNNSIEKSGWLFIQLYHSFVSSVFSLFMSRTSINGLLGRGSMFVFSPDQFQRLLKINPDWKTHRLLDLGAGDGEVTKIMSPHFEEIYATELSETMIWQLQKKKYRVLGINEWQNTGFQYDVISCLNLLDRCDQPLTLLKDIRSVLEPTRGRVILALVLPFHPYVENVGGKWEKPSEILEIKGQNWEEQVNSLPEVFRKAGFVIEAFTRLPYLCEGDMYNDYYVLDDAVFVLKPV

SEQ ID NO: 129 - protein, 533 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSGSQSEVAPSPQSPRSPEMGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 130 - protein, 273 aa, Homo sapiens
MSGSQSEVAPSPQSPRSPEMGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 131 - protein, 376 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 132 - protein, 116 aa, Homo sapiens
MKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 133 - protein, 468 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNSGISQVFQRELTCPICMNYFIDPVTIDCGHSFCRPCFYLNWQDIPILTQCFECIKTIQQRNLKTNIRLKKMASLARKASLWLFLSSEEQMCGIHRETKKMFCEVDRSLLCLLCSSSQEHRYHRHCPAEWAAEEHWEKLLKKMQSLWEKACENQRNLNVETTRISHWKAFGDILYRSESVLLHMPQPLNLALRAGPITGLRDRLNQF

SEQ ID NO: 134 - protein, 208 aa, Homo sapiens
MNSGISQVFQRELTCPICMNYFIDPVTIDCGHSFCRPCFYLNWQDIPILTQCFECIKTIQQRNLKTNIRLKKMASLARKASLWLFLSSEEQMCGIHRETKKMFCEVDRSLLCLLCSSSQEHRYHRHCPAEWAAEEHWEKLLKKMQSLWEKACENQRNLNVETTRISHWKAFGDILYRSESVLLHMPQPLNLALRAGPITGLRDRLNQF

SEQ ID NO: 135 - protein, 460 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSYALSHTNRKLTLEPKITVNAKIIVVGASSVGISFLETLVFCSHMKFNNLTLISTHGLPGKKLLDTEQRKFLASDHCFNDKDYALMSLCSWVNVVVGRMTGIDRAAKHVVLSTDEIVPYDHLILCTGQQYQVPCPTEADISQHLTNREVPNSSQRRYTGKVPCNHFTLNEEEDCFKALIWIRNNSITTEDGGRASVLFC

SEQ ID NO: 136 - protein, 200 aa, Homo sapiens [sequence omitted]

SEQ ID NO: 137 - protein, 725 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKPTGTDPRILSIAAEVAKSPEQNVPVILLKLKEIINITPLGSSELKKIKQDIYCYDLIQYCLLVLSQDYSRIQGGWTTISQLTQILSHCCVGLEPGEDAEEFYNELLPSAAENFLVLGRQLQTCFINAAKAEEKDELLHFFQIVTDSLFWLLGGHVELIQNVLQSDHFLHLLQADNVQIGSAVMMMLQNILQINRSKRSKMLLEINRQKEEEDLKLQLQLQRQRAMRLSRELQLSMLEIVHPGQVEKHYREMEEKSALIIQKHWRGYRERKNFHQQRQSLIEYKAAVTLQRAALKFLAKYRKKKKLFAPWRGLQELTDARRVELKKRVDDYVRRHLGSPMSDVVSRELHAQAQERLQHYFMGRALEERAQQHREALIAQISTNVEQLMKAPSLKEAEGKEPELFLSRSRPVAAKAKQAHLTTLKHIQAPWWKKLGEESGDEIDVPKDELSIELENLFIGGTKPP

SEQ ID NO: 138 - protein, 465 aa, Homo sapiens
MKPTGTDPRILSIAAEVAKSPEQNVPVILLKLKEIINITPLGSSELKKIKQDIYCYDLIQYCLLVLSQDYSRIQGGWTTISQLTQILSHCCVGLEPGEDAEEFYNELLPSAAENFLVLGRQLQTCFINAAKAEEKDELLHFFQIVTDSLFWLLGGHVELIQNVLQSDHFLHLLQADNVQIGSAVMMMLQNILQINRSKRSKMLLEINRQKEEEDLKLQLQLQRQRAMRLSRELQLSMLEIVHPGQVEKHYREMEEKSALIIQKHWRGYRERKNFHQQRQSLIEYKAAVTLQRAALKFLAKYRKKKKLFAPWRGLQELTDARRVELKKRVDDYVRRHLGSPMSDVVSRELHAQAQERLQHYFMGRALEERAQQHREALIAQISTNVEQLMKAPSLKEAEGKEPELFLSRSRPVAAKAKQAHLTTLKHIQAPWWKKLGEESGDEIDVPKDELSIELENLFIGGTKPP

SEQ ID NO: 139 - protein, 759 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEFGLSWVFLVAILKGVQCEVQLVESGGGVVRPGGSLRLSCATSGFTFDDSGASWVRQAPGKGLEWVSSINWNGGSTNYADSVKGRFTISRDNAKNSLYLQMNSLRVEDTALYYCARDPTKYCSGGSCLGYYMDVWGKGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 140 - protein, 499 aa, Homo sapiens [sequence omitted]

SEQ ID NO: 141 - protein, 542 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSLLATLGLELDRALLPASGLGWLVDYGKLPPAPAPLAPYEVLGGALEGGLPVGGEPLAGDGFSDWMTERVDFTALLPLEPPLPPGTLPQPSPTPPDLEAMASLLKKELEQMEDFFLDAPLLPPPSPPPLPPPPLPPAPSLPLSLPSFDLPQPPVLDTLDLLAIYCRNEAGQEEVGMPPLPPPQQPPPPSPPQPSRLAPYPHPATTRGDRKQKKRDQNKSAALRYRQRKRAEGEALEGECQGLEARNRELKERAESVEREIQYVKDLLIEVYKARSQRTRSC

SEQ ID NO: 142 - protein, 282 aa, Homo sapiens
MSLLATLGLELDRALLPASGLGWLVDYGKLPPAPAPLAPYEVLGGALEGGLPVGGEPLAGDGFSDWMTERVDFTALLPLEPPLPPGTLPQPSPTPPDLEAMASLLKKELEQMEDFFLDAPLLPPPSPPPLPPPPLPPAPSLPLSLPSFDLPQPPVLDTLDLLAIYCRNEAGQEEVGMPPLPPPQQPPPPSPPQPSRLAPYPHPATTRGDRKQKKRDQNKSAALRYRQRKRAEGEALEGECQGLEARNRELKERAESVEREIQYVKDLLIEVYKARSQRTRSC

SEQ ID NO: 143 - protein, 713 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSKISEAVKRARAAFSSGRTRPLQFRIQQLEALQRLIQEQEQELVGALAADLHKNEWNAYYEEVVYVLEEIEYMIQKLPEWAADEPVEKTPQTQQDELYIHSEPLGVVLVIGTWNYPFNLTIQPMVGAIAAGNAVVLKPSELSENMASLLATIIPQYLDKDLYPVINGGVPETTELLKERFDHILYTGSTGVGKIIMTAAAKHLTPVTLELGGKSPCYVDKNCDLDVACRRIAWGKFMNSGQTCVAPDYILCDPSIQNQIVEKLKKSLKEFYGEDAKKSRDYGRIISARHFQRVMGLIEGQKVAYGGTGDAATRYIAPTILTDVDPQSPVMQEEIFGPVLPIVCVRSLEEAIQFINQREKPLALYMFSSNDKVIKKMIAETSSGGVAANDVIVHITLHSLPFGGVGNSGMGSYHGKKSFETFSHRRSCLVRPLMNDEGLKVRYPPSPAKMTQH

SEQ ID NO: 144 - protein, 453 aa, Homo sapiens [sequence omitted]

SEQ ID NO: 145 - protein, 591 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMENPSPAAALGKALCALLLATLGAAGQPLGGESICSARAPAKYSITFTGKWSQTAFPKQYPLFRPPAQWSSLLGAAHSSDYSMWRKNQYVSNGLRDFAERGEAWALMKEIEAAGEALQSVHEVFSAPAVPSGTGQTSAELEVQRRHSLVSFVVRIVPSPDWFVGVDSLDLCDGDRWREQAALDLYPYDAGTDSGFTFSSPNFATIPQDTVTEITSSSPSHPANSFYYPRLKALPPIARVTLLRLRQSPRAFIPPAPVLPSRDNEIVDSASVPETPLDCEVSLWSSWGLCGGHCGRLGTKSRTRYVRVQPANNGSPCPELEEEAECVPDNCV

SEQ ID NO: 146 - protein, 331 aa, Homo sapiens [sequence omitted]

SEQ ID NO: 147 - protein, 595 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 148 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 149 - protein, 606 aa, synthetic construct; identical to SEQ ID NO: 37

SEQ ID NO: 150 - protein, 346 aa, Homo sapiens; identical to SEQ ID NO: 38

SEQ ID NO: 151 - protein, 782 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSGFNFGGTGAPTGGFTFGTAKTATTTPATGFSFSTSGTGGFNFGAPFQPATSTPSTGLFSLATQTPATQTTGFTFGTATLASGGTGFSLGIGASKLNLSNTAATPAMANPSGFGLGSSNLTNAISSTVTSSQGTAPTGFVFGPSTTSVAPATTSGGFSFTGGSTAQPSGFNIGSAGNSAQPTAPATLPFTPATPAATTAGATQPAAPTPTATITSTGPSLFASIATAPTSSATTGLSLCTPVTTAGAPTAGTQGFSLKAPGAASGTSTTTSTAATATATTTSSSSTTGFALNLKPLAPAGIPSNTAAAVTAPPGPGAAAGAAASSAMTYAQLESLINKWSLELEDQERHFLQQATQVNAWDRTLIENGEKITSLHREVEKVKLDQKRLDQELDFILSQQKELEDLLSPLEELVKEQSGTIYLQHADEEREKTYKLAENIDAQLKRMAQDLKDIIEHLNTSGAPADTSDPLQQICKILNAHMDSLQWIDQNSALLQRKVEEVTKVCEGRRKEQERSFRITFD

SEQ ID NO: 152 - protein, 522 aa, Homo sapiens
MSGFNFGGTGAPTGGFTFGTAKTATTTPATGFSFSTSGTGGFNFGAPFQPATSTPSTGLFSLATQTPATQTTGFTFGTATLASGGTGFSLGIGASKLNLSNTAATPAMANPSGFGLGSSNLTNAISSTVTSSQGTAPTGFVFGPSTTSVAPATTSGGFSFTGGSTAQPSGFNIGSAGNSAQPTAPATLPFTPATPAATTAGATQPAAPTPTATITSTGPSLFASIATAPTSSATTGLSLCTPVTTAGAPTAGTQGFSLKAPGAASGTSTTTSTAATATATTTSSSSTTGFALNLKPLAPAGIPSNTAAAVTAPPGPGAAAGAAASSAMTYAQLESLINKWSLELEDQERHFLQQATQVNAWDRTLIENGEKITSLHREVEKVKLDQKRLDQELDFILSQQKELEDLLSPLEELVKEQSGTIYLQHADEEREKTYKLAENIDAQLKRMAQDLKDIIEHLNTSGAPADTSDPLQQICKILNAHMDSLQWIDQNSALLQRKVEEVTKVCEGRRKEQERSFRITFD

SEQ ID NO: 153 - protein, 616 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTVATGDPADEAAALPGHPQDTYDPEADHECCERVVINISGLRFETQLKTLAQFPETLLGDPKKRMRYFDPLRNEYFFDRNRPSFDAILYYYQSGGRLRRPVNVPLDIFSEEIRFYELGEEAMEMFREDEGYIKEEERPLPENEFQRQVWLLFEYPESSGPARIIAIVSVMVILISIVSFCLETLPIFRDENEDMHGSGVTFHTYSNSTIGYQQSTSFTDPFFIVETLCIIWFSFEFLVRFFACPSKAGFFTNIMNIIDIVAIIPYFITLGTELAEKPEDAQQGQQAMSLAILRVIRLERRPLQSQKSKRGRQHLNTSHDCTLGINLVAGMTVQWTRASGPDDRQTPAVTTLHRMY

SEQ ID NO: 154 - protein, 356 aa, Homo sapiens
MTVATGDPADEAAALPGHPQDTYDPEADHECCERVVINISGLRFETQLKTLAQFPETLLGDPKKRMRYFDPLRNEYFFDRNRPSFDAILYYYQSGGRLRRPVNVPLDIFSEEIRFYELGEEAMEMFREDEGYIKEEERPLPENEFQRQVWLLFEYPESSGPARIIAIVSVMVILISIVSFCLETLPIFRDENEDMHGSGVTFHTYSNSTIGYQQSTSFTDPFFIVETLCIIWFSFEFLVRFFACPSKAGFFTNIMNIIDIVAIIPYFITLGTELAEKPEDAQQGQQAMSLAILRVIRLERRPLQSQKSKRGRQHLNTSHDCTLGINLVAGMTVQWTRASGPDDRQTPAVTTLHRMY

SEQ ID NO: 155 - protein, 862 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMRAQEDLEGRTQHETTRDPSTPLPTEPKFDMLYKIEDVPPWYLCILLGFQHYLTCFSGTIAVPFLLAEALCVGHDQHMVSQLIGTIFTCVGITTLIQTTVGIRLPLFQASAFAFLVPAKAILALERWKCPPEEEIYGNWSLPLNTSHIWHPRIREVGLHVQGAIMVSSVVEVVIGLLGLPGALLNYIGPLTVTPTVSLIGLSVFQAAGDRAGSHWGISACSILLIILFSQYLRNLTFLLPVYRWGKGLTLLRIQIFKMFPIMLAIMTVWLLCYVLTLTDVLPTDPKAYGFQARTDARGDIMAIAPWIRIPYPCQWGLPTVTAAAVLGMFSATLAGIIESIGDYYACARLAGAPPPPVHAINRGIFTEGICCIIAGLLGTGNGSTSSSPNIGVLGITKVGSRRVVQYGAAIMLVLGTIGKFTALFASLPDPILGGMFCTLFGMITAVGLSNLQFVDMNSSRNLFVLGFSMFFGLTLPNYLESNPGAINTGILEVDQILIVLLTTEMFVGGCLAFILDNTVPGSPEERGLIQWKAGAHANSDMSSSLKSYDFPIGMGIVKRITFLKYIPICPVFKGFSSSSKDQIAIPEDTPENTETASVCTKV

SEQ ID NO: 156 - protein, 602 aa, Homo sapiens
MRAQEDLEGRTQHETTRDPSTPLPTEPKFDMLYKIEDVPPWYLCILLGFQHYLTCFSGTIAVPFLLAEALCVGHDQHMVSQLIGTIFTCVGITTLIQTTVGIRLPLFQASAFAFLVPAKAILALERWKCPPEEEIYGNWSLPLNTSHIWHPRIREVGLHVQGAIMVSSVVEVVIGLLGLPGALLNYIGPLTVTPTVSLIGLSVFQAAGDRAGSHWGISACSILLIILFSQYLRNLTFLLPVYRWGKGLTLLRIQIFKMFPIMLAIMTVWLLCYVLTLTDVLPTDPKAYGFQARTDARGDIMAIAPWIRIPYPCQWGLPTVTAAAVLGMFSATLAGIIESIGDYYACARLAGAPPPPVHAINRGIFTEGICCIIAGLLGTGNGSTSSSPNIGVLGITKVGSRRVVQYGAAIMLVLGTIGKFTALFASLPDPILGGMFCTLFGMITAVGLSNLQFVDMNSSRNLFVLGFSMFFGLTLPNYLESNPGAINTGILEVDQILIVLLTTEMFVGGCLAFILDNTVPGSPEERGLIQWKAGAHANSDMSSSLKSYDFPIGMGIVKRITFLKYIPICPVFKGFSSSSKDQIAIPEDTPENTETASVCTKV

SEQ ID NO: 157 - protein, 899 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLSYGERLGSPAVSPLPVRGGHVMRGTAFAYVPSPQVLHRIPGTSAYAFPSLGPVALAEHTCPCGEVLERHEPLPAKLALEEEQKPESRLVPKLRQAGAMLLKVPLMLTFLYLFVCSLDMLSSAFQLAGGKVAGDIFKDNAILSNPVAGLVVGILVTVLVQSSSTSTSIIVSMVSSGLLEVSSAIPIIMGSNIGTSVTNTIVALMQAGDRTDFRRAFAGATVHDCFNWLSVLVLLPLEAATGYLHHITRLVVASFNIHGGRDAPDLLKIITEPFTKLIIQLDESVITSIATGDESLRNHSLIQIWCHPDSLQAPTSMSRAEANSSQTLGNATMEKCNHIFVDTGLPDLAVGLILLAGALVLLCTCLILLVKMLNSLLKGQVAKVIQKVINTDFPAPFTWVTGYFAMVVGASMTFVVQSSSVFTSAITPLIGLGVISIERAYPLTLGSNIGTTTTAILAALASPREKLSSAFQIALCHFFFNISGILLWYPVPCTRLPIRMAKALGKRTAKYRWFAVLYLLVCFLLLPSLVFGISMAGWQVMVGVGTPFGALLAFVVLINVLQSRSPGHLPKWLQTWDFLPRWMHSLKPLDHLITRATLCCARPEPRSPPLPPRVFLEELPPATPSPRLALPAHHNATRL

SEQ ID NO: 158 - protein, 639 aa, Homo sapiens
MLSYGERLGSPAVSPLPVRGGHVMRGTAFAYVPSPQVLHRIPGTSAYAFPSLGPVALAEHTCPCGEVLERHEPLPAKLALEEEQKPESRLVPKLRQAGAMLLKVPLMLTFLYLFVCSLDMLSSAFQLAGGKVAGDIFKDNAILSNPVAGLVVGILVTVLVQSSSTSTSIIVSMVSSGLLEVSSAIPIIMGSNIGTSVTNTIVALMQAGDRTDFRRAFAGATVHDCFNWLSVLVLLPLEAATGYLHHITRLVVASFNIHGGRDAPDLLKIITEPFTKLIIQLDESVITSIATGDESLRNHSLIQIWCHPDSLQAPTSMSRAEANSSQTLGNATMEKCNHIFVDTGLPDLAVGLILLAGALVLLCTCLILLVKMLNSLLKGQVAKVIQKVINTDFPAPFTWVTGYFAMVVGASMTFVVQSSSVFTSAITPLIGLGVISIERAYPLTLGSNIGTTTTAILAALASPREKLSSAFQIALCHFFFNISGILLWYPVPCTRLPIRMAKALGKRTAKYRWFAVLYLLVCFLLLPSLVFGISMAGWQVMVGVGTPFGALLAFVVLINVLQSRSPGHLPKWLQTWDFLPRWMHSLKPLDHLITRATLCCARPEPRSPPLPPRVFLEELPPATPSPRLALPAHHNATRL

SEQ ID NO: 159 - protein, 437 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLYEFFVKVYPSCRGGKVVLDQTGVSKGYGFVKFTDELEQKRALTECQGAVGLGSKPVRLSVAIPKASRVKPVEYSQMYSYSYNQYYQQYQNYYAQWGYDQNTGSYSYSYPQYGYTQSTMQTYEEVGDDALEDPMPQLDVTEANKEFMEQSEELYDALMDCHWQPLDTVSSEIPAMM

SEQ ID NO: 160 - protein, 177 aa, Homo sapiens
MLYEFFVKVYPSCRGGKVVLDQTGVSKGYGFVKFTDELEQKRALTECQGAVGLGSKPVRLSVAIPKASRVKPVEYSQMYSYSYNQYYQQYQNYYAQWGYDQNTGSYSYSYPQYGYTQSTMQTYEEVGDDALEDPMPQLDVTEANKEFMEQSEELYDALMDCHWQPLDTVSSEIPAMM

SEQ ID NO: 161 - protein, 506 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDLVQVFHCHFNSNFFPLFLEHLLSDHISLSTGSILLGLSYVAPHLNNLYFLSLCRRWGVYAVVRGLFLLKLGLSLLMLLAGPDHLSLLCLFIASNRVFTEGTCKLLTLVVTDLVDEDLVLNHRKQAASALLFGMVALVTKPGQTFAPLLGTWLLCFYTGHDLFQQSLITPVGSAHPWPEPPAPAPAQAPTLRQGCFYLLVLVPITCALLQLFTWSQSTLHGRRLHMVKAQRQNLSQAQTLDVKMV

SEQ ID NO: 162 - protein, 246 aa, Homo sapiens
MDLVQVFHCHFNSNFFPLFLEHLLSDHISLSTGSILLGLSYVAPHLNNLYFLSLCRRWGVYAVVRGLFLLKLGLSLLMLLAGPDHLSLLCLFIASNRVFTEGTCKLLTLVVTDLVDEDLVLNHRKQAASALLFGMVALVTKPGQTFAPLLGTWLLCFYTGHDLFQQSLITPVGSAHPWPEPPAPAPAQAPTLRQGCFYLLVLVPITCALLQLFTWSQSTLHGRRLHMVKAQRQNLSQAQTLDVKMV

SEQ ID NO: 163 - protein, 481 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSDGDYDYLIKFLALGDSGVGKTSVLYQYTDGKFNSKFITTVGIDFREKRVVYRASGPDGATGRGQRIHLQLWDTAGQERFRSLTTAFFRDAMGFLLLFDLTNEQSFLNVRNWISQLQMHAYCENPDIVLCGNKSDLEDQRVVKEEEAIALAEKYGIPYFETSAANGTNISQAIEMLLDLIMKRMERCVDKSWIPEGVVRSNGHASTDQLSEEKEKGACGC

SEQ ID NO: 164 - protein, 221 aa, Homo sapiens
MSDGDYDYLIKFLALGDSGVGKTSVLYQYTDGKFNSKFITTVGIDFREKRVVYRASGPDGATGRGQRIHLQLWDTAGQERFRSLTTAFFRDAMGFLLLFDLTNEQSFLNVRNWISQLQMHAYCENPDIVLCGNKSDLEDQRVVKEEEAIALAEKYGIPYFETSAANGTNISQAIEMLLDLIMKRMERCVDKSWIPEGVVRSNGHASTDQLSEEKEKGACGC

SEQ ID NO: 165 - protein, 604 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKDLGAEHLAGHEGVQLLGLLNVYLEQEERFQPREKGLSLIEATPENDNTLCPGLRNAKVEDLRSLANFFGSCTETFVLAVNILDRFLALMKVKPKHLSCIGVCSFLLAARIVEEDCNIPSTHDVIRISQCKCTASDIKRMEKIISEKLHYELEATTALNFLHLYHTIILCHTSERKEILSLDKLEAQLKACNCRLIFSKAKPSVLALCLLNLEVETLKSVELLEILLLVKKHSKINDTEFFYWRELVSKCLAEYSSPECCKPDLKKLVWIVSRRTAQNLHNSYYSVPELPTIPEGGCFDESESSVAQAGVQWPDLSSFQPPPTRFKRFSCLSLRSSWDYSSWT

SEQ ID NO: 166 - protein, 344 aa, Homo sapiens
MKDLGAEHLAGHEGVQLLGLLNVYLEQEERFQPREKGLSLIEATPENDNTLCPGLRNAKVEDLRSLANFFGSCTETFVLAVNILDRFLALMKVKPKHLSCIGVCSFLLAARIVEEDCNIPSTHDVIRISQCKCTASDIKRMEKIISEKLHYELEATTALNFLHLYHTIILCHTSERKEILSLDKLEAQLKACNCRLIFSKAKPSVLALCLLNLEVETLKSVELLEILLLVKKHSKINDTEFFYWRELVSKCLAEYSSPECCKPDLKKLVWIVSRRTAQNLHNSYYSVPELPTIPEGGCFDESESSVAQAGVQWPDLSSFQPPPTRFKRFSCLSLRSSWDYSSWT

SEQ ID NO: 167 - protein, 507 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 168 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 169 - protein, 1006 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAGSRGAGRTAAPSVRPEKRRSEPELEPEPEPEPPLLCTSPLSHSTGSDSGVSDSEESVFSGLEDSGSDSSEDDDEGDEEGEDGALDDEGHSGIKKTTEEQVQASTPCPRTEMASARIGDEYAEDSSDEEDIRNTVGNVPLEWYDDFPHVGYDLDGRRIYKPLRTRDELDQFLDKMDDPDYWRTVQDPMTGRDLRLTDEQVALVRRLQSGQFGDVGFNPYEPAVDFFSGDVMIHPVTNRPADKRSFIPSLVEKEKVSRMVHAIKMGWIQPRRPRDPTPSFYDLWAQEDPNAVLGRHKMHVPAPKLALPGHAESYNPPPEYLLSEEERLAWEQQEPGERKLSFLPRKFPSLRAVPAYGRFIQERFERCLDLYLCPRQRKMRVNVDPEDLIPKLPRPRDLQPFPTCQALVYRGHSDLVRCLSVSPGGQWLVSGSDDGSLRLWEVATARCVRTVPVGGVVKSVAWNPSPAVCLVAAAVEDSVLLLNPALGDRLVAGSTDQLLSAFVPPEEPPLQPARWLEASEEERQVGLRLRICHGKPVTQVTWHGRGDYLAVVLATQGHTQVLIHQLSRRRSQSPFRRSHGQVQRVAFHPARPFLLVASQRSVRLYHLLRQELTKKLMPNCKWVSSLAVHPAGDNVICGSYDSKLVWFDLDLSTKPYRMLRHHKKALRAVAFHPRYPLFASGSDDGSVIVCHGMVYNDLLQNPLLVPVKVLKGHVLTRDLGVLDVIFHPTQPWVFSSGADGTVRLFT

SEQ ID NO: 170 - protein, 746 aa, Homo sapiens
MAGSRGAGRTAAPSVRPEKRRSEPELEPEPEPEPPLLCTSPLSHSTGSDSGVSDSEESVFSGLEDSGSDSSEDDDEGDEEGEDGALDDEGHSGIKKTTEEQVQASTPCPRTEMASARIGDEYAEDSSDEEDIRNTVGNVPLEWYDDFPHVGYDLDGRRIYKPLRTRDELDQFLDKMDDPDYWRTVQDPMTGRDLRLTDEQVALVRRLQSGQFGDVGFNPYEPAVDFFSGDVMIHPVTNRPADKRSFIPSLVEKEKVSRMVHAIKMGWIQPRRPRDPTPSFYDLWAQEDPNAVLGRHKMHVPAPKLALPGHAESYNPPPEYLLSEEERLAWEQQEPGERKLSFLPRKFPSLRAVPAYGRFIQERFERCLDLYLCPRQRKMRVNVDPEDLIPKLPRPRDLQPFPTCQALVYRGHSDLVRCLSVSPGGQWLVSGSDDGSLRLWEVATARCVRTVPVGGVVKSVAWNPSPAVCLVAAAVEDSVLLLNPALGDRLVAGSTDQLLSAFVPPEEPPLQPARWLEASEEERQVGLRLRICHGKPVTQVTWHGRGDYLAVVLATQGHTQVLIHQLSRRRSQSPFRRSHGQVQRVAFHPARPFLLVASQRSVRLYHLLRQELTKKLMPNCKWVSSLAVHPAGDNVICGSYDSKLVWFDLDLSTKPYRMLRHHKKALRAVAFHPRYPLFASGSDDGSVIVCHGMVYNDLLQNPLLVPVKVLKGHVLTRDLGVLDVIFHPTQPWVFSSGADGTVRLFT

SEQ ID NO: 171 - protein, 681 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPFLHGFRRIIFEYQPLVDAILGSLGIQDPERQESLDRPSYVASEESRILVLTELLERKAHSPFYQEGVSNALLKMAELGLTRAADVLLRHGANLNFEDPVTYYTALHIAVLRNQPDMVELLVHHGADVNRRDRIHESSPLDLASEEPERLPCLQRLLDLGADVNAADKHGKTALLHALASSDGVQIHNTENIRLLLEGGADVKATTKDGDTVFTCIIFLLGETVGGDKEEAQMINRFCFQVTRLLLAHGADPSECPAHESLTHICLKSFKLHFPLLRFLLESGAAYNCSLHGASCWSGFHIIFERLCSHPGCTEDESHADLLRKAETVLDLMVTNSQKLQLPENFDIHPVGSLAEKIQALHFSLRQLESYPPPLKHLCRVAIRLYLQPWPVDVKVKALPLPDRLKWYLLSEHSGSVEDDI

SEQ ID NO: 172 - protein, 421 aa, Homo sapiens
MPFLHGFRRIIFEYQPLVDAILGSLGIQDPERQESLDRPSYVASEESRILVLTELLERKAHSPFYQEGVSNALLKMAELGLTRAADVLLRHGANLNFEDPVTYYTALHIAVLRNQPDMVELLVHHGADVNRRDRIHESSPLDLASEEPERLPCLQRLLDLGADVNAADKHGKTALLHALASSDGVQIHNTENIRLLLEGGADVKATTKDGDTVFTCIIFLLGETVGGDKEEAQMINRFCFQVTRLLLAHGADPSECPAHESLTHICLKSFKLHFPLLRFLLESGAAYNCSLHGASCWSGFHIIFERLCSHPGCTEDESHADLLRKAETVLDLMVTNSQKLQLPENFDIHPVGSLAEKIQALHFSLRQLESYPPPLKHLCRVAIRLYLQPWPVDVKVKALPLPDRLKWYLLSEHSGSVEDDI

SEQ ID NO: 173 - protein, 771 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKNYEQALQDASAACQNEPLLIKGHQVKAQALSGLGRSKEVLKEFLYCLALNPECNSVKKEAQKVMCEVLFSATANVHENLTSSIQSRLKAQGHSHMNAQALLEEGDAGSSENSSEKSDMLGNTNSSVLYFILGLHFEEDKKALESILPTAPSAGLKRQFPDDVEDAPDLNAPGKIPKKDLSPQRSPNSETEESQGLSLDVTDFECALCMRLLFEPVTTPCGHTFCLKCLERCLDHAPHCPLCKDKLSELLASRNFNITVLAEELIFRYLPDELSDRKRIYDEEMSELSNLTRDVPIFVCAMAFPTVPCPLHVFEPRYRLMIRRCMETGTKRFGMCLSAEHAGLSEYGCMLEIKDVRTFPDGSSVVDAIGISRFRVLSHRHRDGYNTADIEYLEDEKVEGPEYEELAALHDSVHQQSVSWFASLQDRMKEQILSHFGVMPDREPEPQSNPSGPAWSWWILAVLPLERKAQLAILGMTSLKERLLAIRRILVIITRKMNSRQELANARERNN

SEQ ID NO: 174 - protein, 511 aa, Homo sapiens
MKNYEQALQDASAACQNEPLLIKGHQVKAQALSGLGRSKEVLKEFLYCLALNPECNSVKKEAQKVMCEVLFSATANVHENLTSSIQSRLKAQGHSHMNAQALLEEGDAGSSENSSEKSDMLGNTNSSVLYFILGLHFEEDKKALESILPTAPSAGLKRQFPDDVEDAPDLNAPGKIPKKDLSPQRSPNSETEESQGLSLDVTDFECALCMRLLFEPVTTPCGHTFCLKCLERCLDHAPHCPLCKDKLSELLASRNFNITVLAEELIFRYLPDELSDRKRIYDEEMSELSNLTRDVPIFVCAMAFPTVPCPLHVFEPRYRLMIRRCMETGTKRFGMCLSAEHAGLSEYGCMLEIKDVRTFPDGSSVVDAIGISRFRVLSHRHRDGYNTADIEYLEDEKVEGPEYEELAALHDSVHQQSVSWFASLQDRMKEQILSHFGVMPDREPEPQSNPSGPAWSWWILAVLPLERKAQLAILGMTSLKERLLAIRRILVIITRKMNSRQELANARERNN

SEQ ID NO: 175 - protein, 1048 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDASLEKIADPTLAEMGKNLKEAVKMLEDSQRRTEEENGKKLISGDIPGPLQGSGQDMVSILQLVQNLMHGDEDEEPQSPRIQNIGEQGHMALLGHSLGAYISTLDKEKLRKLTTRILSDTTLWLCRIFRYENGCAYFHEEEREGLAKICRLAIHSRYEDFVVDGFNVLYNKKPVIYLSAAARPGLGQYLCNQLGLPFPCLCRVPCNTVFGSQHQMDVAFLEKLIKDDIERGRLPLLLVANAGTAAVGHTDKIGRLKELCEQYGIWLHVEGVNLATLALGYVSSSVLAAAKCDSMTMTPGPWLGLPAVPAVTLYKHDDPALTLVAGLTSNKPTDKLRALPLWLSLQYLGLDGFVERIKHACQLSQRLQESLKEVNYIKILVEDELSSPVVVFRFFQELPGSDPVFKAVPVPNMTPSGVGRERHSCDALNRWLGEQLKQLVPASGLTVMDLEAEGTCLRFSPLMTAAVLGTRGEDVDQLVACIESKLPVLCCTLQLREEFKQEVEATAGLLYVDDPNWSGIGVVRYEHANDDKSSLKSDPEGENIHAGLLKKLNELESDLTFKIGPEYKSMKSCLYVGMASDNVDAAELVETIAATAREIEENSRLLENMTEVVRKGIQEAQVELQKASEERLLEEGVLRQIPVVGSVLNWFSPVQALQKGRTFNLTAGSLESTEPIYVYKAQGAGVTLPPTPSGSRTKQRLPGQKPFKRSLRGSDALSETSSVSHIEDLEKVERLSSGPEQITLEASSTEGHPGAPSPQHTDQTEAFQKGVPHPEDDHSQVEGPESLR

SEQ ID NO: 176 - protein, 788 aa, Homo sapiens
MDASLEKIADPTLAEMGKNLKEAVKMLEDSQRRTEEENGKKLISGDIPGPLQGSGQDMVSILQLVQNLMHGDEDEEPQSPRIQNIGEQGHMALLGHSLGAYISTLDKEKLRKLTTRILSDTTLWLCRIFRYENGCAYFHEEEREGLAKICRLAIHSRYEDFVVDGFNVLYNKKPVIYLSAAARPGLGQYLCNQLGLPFPCLCRVPCNTVFGSQHQMDVAFLEKLIKDDIERGRLPLLLVANAGTAAVGHTDKIGRLKELCEQYGIWLHVEGVNLATLALGYVSSSVLAAAKCDSMTMTPGPWLGLPAVPAVTLYKHDDPALTLVAGLTSNKPTDKLRALPLWLSLQYLGLDGFVERIKHACQLSQRLQESLKEVNYIKILVEDELSSPVVVFRFFQELPGSDPVFKAVPVPNMTPSGVGRERHSCDALNRWLGEQLKQLVPASGLTVMDLEAEGTCLRFSPLMTAAVLGTRGEDVDQLVACIESKLPVLCCTLQLREEFKQEVEATAGLLYVDDPNWSGIGVVRYEHANDDKSSLKSDPEGENIHAGLLKKLNELESDLTFKIGPEYKSMKSCLYVGMASDNVDAAELVETIAATAREIEENSRLLENMTEVVRKGIQEAQVELQKASEERLLEEGVLRQIPVVGSVLNWFSPVQALQKGRTFNLTAGSLESTEPIYVYKAQGAGVTLPPTPSGSRTKQRLPGQKPFKRSLRGSDALSETSSVSHIEDLEKVERLSSGPEQITLEASSTEGHPGAPSPQHTDQTEAFQKGVPHPEDDHSQVEGPESLR

SEQ ID NO: 177 - protein, 449 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 178 - protein, 189 aa, Homo sapiens
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 179 - protein, 377 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPSCSCALMAPCGPAAGPAAVERTQQVARGEPGSARGQLQVSPEMSITHKEKENAHLKEILLFVNAEAFSQPQPHSAPVCEGQQLTGKFSTSVLTRAGGDASPCSWERLLCYGWSHC

SEQ ID NO: 180 - protein, 117 aa, Homo sapiens
MPSCSCALMAPCGPAAGPAAVERTQQVARGEPGSARGQLQVSPEMSITHKEKENAHLKEILLFVNAEAFSQPQPHSAPVCEGQQLTGKFSTSVLTRAGGDASPCSWERLLCYGWSHC

SEQ ID NO: 181 - protein, 537 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNGFASLLRRNQFILLVLFLLQIQSLGLDIDSRPTAEVCATHTISPGPKGDDGEKGDPGEEGKHGKVGRMGPKGIKGELGDMGDQGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 182 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDQGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 183 - protein, 768 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 184 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 185 - protein, 944 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSAIPAEESDQLLIRPLGAGQEVGRSCIILEFKGRKIMLDCGIHPGLEGMDALPYIDLIDPAEIDLLLISHFHLDHCGALPWFLQKTSFKGRTFMTHATKAIYRWLLSDYVKVSNISADDMLYTETDLEESMDKIETINFHGVKEVAGIKFWCYHAGHVLGAAMFMIEIAGVKLLYTGDFSRQEDRHLMAAEIPNIKPDILIIESTYGTHIHEKREEREARFCNTVHDIVNRGGRGLIPVFALGRAQELLLILDEYWQNHPELHDIPIYYASSLAKKCMAVYQTYVNAMNDKIRKQININNPFVFKHISNLKSMDHFDDIGPSVVMASPGMMQSGLSRELFESWCTDKRNGVIIAGYCVEGTLAKHIMSEPEEITTMSGQKLPLKMSVDYISFSAHTDYQQTSEFIRALKPPHVILVHGEQNEMARLKAALIREYEDNDEVHIEVHNPRNTEAVTLNFRGEKLAKVMGFLADKKPEQGQRVSGILVKRNFNYHILSPCDLSNYTDLAMSTVKQTQAIPYTGPFNLLCYQLQKLTGDVEELEIQEKPALKVFKNITVIQEPGMVVLEWLANPSNDMYADTVTTVILEVQSNPKIRKGAVQKVSKKLEMHVYSKRLEIMLQDIFGEDCVSVKDDSILSVTVDGKTANLNLETRTVECEEGSEDDESLREMVELAAQRLYEALTPVH

SEQ ID NO: 186 - protein, 684 aa, Homo sapiens [sequence omitted]

SEQ ID NO: 187 - protein, 724 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 188 - protein, 464 aa, Homo sapiens
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 189 - protein, 410 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLGAALRRCAVAATTRADPRGLLHSARTPGPAVAIQSVRCYSHGSQETDEEFDARWVTYFNKPDIDAWELRKGINTLVTYDMVPEPKIIDAALRACRRLNDFASTVRILEVVKDKAGPHKEIYPYVIQELRPTLNELGISTPEELGLDKV

SEQ ID NO: 190 - protein, 150 aa, Homo sapiens
MLGAALRRCAVAATTRADPRGLLHSARTPGPAVAIQSVRCYSHGSQETDEEFDARWVTYFNKPDIDAWELRKGINTLVTYDMVPEPKIIDAALRACRRLNDFASTVRILEVVKDKAGPHKEIYPYVIQELRPTLNELGISTPEELGLDKV

SEQ ID NO: 191 - protein, 1309 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 192 - protein, 1049 aa, Homo sapiens
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 193 - protein, 356 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSDNDDIEVESDEEQPRFQSAADKRAHHNALERKRRDHIKDSFHSLRDSVPSLQGEKLYFLFWKLCTPVLHRQSLMQKCHTFISSYQVHKKKECKI

SEQ ID NO: 194 - protein, 96 aa, Homo sapiens
MSDNDDIEVESDEEQPRFQSAADKRAHHNALERKRRDHIKDSFHSLRDSVPSLQGEKLYFLFWKLCTPVLHRQSLMQKCHTFISSYQVHKKKECKI

SEQ ID NO: 195 - protein, 635 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDELQDVQLTEIKPLLNDKNGTRNFQDFDCQEHDIETTHGVVHVTIRGLPKGNRPVILTYHDIGLNHKSCFNAFFNFEDMQEITQHFAVCHVDAPGQQEGAPSFPTGYQYPTMDELAEMLPPVLTHLSLKSIIGIGVGAGAYILSRFALNHPELVEGLVLINVDPCAKGWIDWAASKLSGLTTNVVDIILAHHFGQEELQANLDLIQTYRMHIAQDINQDNLQLFLNSYNGRRDLEIERPILGQNDNKSKTLKCSTLLVVGDNSPAVEAVVECNSRLNPINTTLLKMADCGGLPQVVQPGKLTEAFKYFLQGMGYIPSASMTRLARSRTHSTSSSLGSGESPFSRSVTSNQSDGTQESCESPDVLDRHQTMEVSC

SEQ ID NO: 196 - protein, 375 aa, Homo sapiens
MDELQDVQLTEIKPLLNDKNGTRNFQDFDCQEHDIETTHGVVHVTIRGLPKGNRPVILTYHDIGLNHKSCFNAFFNFEDMQEITQHFAVCHVDAPGQQEGAPSFPTGYQYPTMDELAEMLPPVLTHLSLKSIIGIGVGAGAYILSRFALNHPELVEGLVLINVDPCAKGWIDWAASKLSGLTTNVVDIILAHHFGQEELQANLDLIQTYRMHIAQDINQDNLQLFLNSYNGRRDLEIERPILGQNDNKSKTLKCSTLLVVGDNSPAVEAVVECNSRLNPINTTLLKMADCGGLPQVVQPGKLTEAFKYFLQGMGYIPSASMTRLARSRTHSTSSSLGSGESPFSRSVTSNQSDGTQESCESPDVLDRHQTMEVSC

SEQ ID NO: 197 - protein, 515 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 198 - protein, 255 aa, Homo sapiens
MFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 199 - protein, 393 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSHTVQTFFSPVNSGQPPNYEMLKEEHEVAVLGGPHNPAPPTSTVIHIRSETSVPDHVVWSLFNTLFMNPCCLGFIAFAYSVKSRDRKMVGDVTGAQAYASTAKCLNIWALILGILMTILLIVIPVLIFQAYG

SEQ ID NO: 200 - protein, 133 aa, Homo sapiens
MSHTVQTFFSPVNSGQPPNYEMLKEEHEVAVLGGPHNPAPPTSTVIHIRSETSVPDHVVWSLFNTLFMNPCCLGFIAFAYSVKSRDRKMVGDVTGAQAYASTAKCLNIWALILGILMTILLIVIPVLIFQAYG

SEQ ID NO: 201 - protein, 683 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNALLLSAWFGHLRILQILVNSGAKIHCESKDGLTLLHCAAQKGHVPVLAFIMEDLEDVALDHVDKLGRTAFHRAAEHGQLDALDFLVGSGCDHNVKDKEGNTALHLAAGRGHMAVLQRLVDIGLDLEEQNAEGLTALHSAAGGSHPDCVQLLLRAGSTVNALTQKNLSCLHYAALSGSEDVSRVLIHAGGCANVVDHQGASPLHLAVRHNFPALVRLLINSDSDVNAVDNRQQTPLHLAAEHAWQDIADMLLIAGVDLNLRDKQGKTALAVAVRSNHVSLVDMIIKADRFYRWEKDHPSDPSGKSLSFKQDHRQETQQLRSVLWRLASRYLQPREWKKLAYSWEFTEAHVDAIEQQWTGTRSYQEHGHRMLLIWLHGVATAGENPSKALFEGLVAIGRRDLAENIRKKANAAPSAPRRCTAM

SEQ ID NO: 202 - protein, 423 aa, Homo sapiens
MNALLLSAWFGHLRILQILVNSGAKIHCESKDGLTLLHCAAQKGHVPVLAFIMEDLEDVALDHVDKLGRTAFHRAAEHGQLDALDFLVGSGCDHNVKDKEGNTALHLAAGRGHMAVLQRLVDIGLDLEEQNAEGLTALHSAAGGSHPDCVQLLLRAGSTVNALTQKNLSCLHYAALSGSEDVSRVLIHAGGCANVVDHQGASPLHLAVRHNFPALVRLLINSDSDVNAVDNRQQTPLHLAAEHAWQDIADMLLIAGVDLNLRDKQGKTALAVAVRSNHVSLVDMIIKADRFYRWEKDHPSDPSGKSLSFKQDHRQETQQLRSVLWRLASRYLQPREWKKLAYSWEFTEAHVDAIEQQWTGTRSYQEHGHRMLLIWLHGVATAGENPSKALFEGLVAIGRRDLAENIRKKANAAPSAPRRCTAM

SEQ ID NO: 203 - protein, 607 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPRGQKSKLRAREKRRQARGGLEDLIDALDILEEEEESPPSASACLKDVFQSSLDGASNNPHGLREAQSTSTSATAASHTRHPEGVNDQMEERPICTQDLEATDSFPRGPVDEKVIILVHYLLYKYQMKEPITKADMLRNVTQMSKSQFPVILSRASEHLELIFGLDLKEVEPNKHIYVLVNKLDLGCDAKLSDETGVPKTGLLMTVLGIIFTNGNCVAEEEVWKVFNTMGLYDGIEHFMFGEPRKLLTKDLVKENYLEYQQVPNSDPPRYQFLWGPRAHAETSKMKVLEFLAKVNDTAPSEFSNWYTEALQDEEERARARVAAKARVSATAGARSKVKSSKSSQLQ

SEQ ID NO: 204 - protein, 347 aa, Homo sapiens
MPRGQKSKLRAREKRRQARGGLEDLIDALDILEEEEESPPSASACLKDVFQSSLDGASNNPHGLREAQSTSTSATAASHTRHPEGVNDQMEERPICTQDLEATDSFPRGPVDEKVIILVHYLLYKYQMKEPITKADMLRNVTQMSKSQFPVILSRASEHLELIFGLDLKEVEPNKHIYVLVNKLDLGCDAKLSDETGVPKTGLLMTVLGIIFTNGNCVAEEEVWKVFNTMGLYDGIEHFMFGEPRKLLTKDLVKENYLEYQQVPNSDPPRYQFLWGPRAHAETSKMKVLEFLAKVNDTAPSEFSNWYTEALQDEEERARARVAAKARVSATAGARSKVKSSKSSQLQ

SEQ ID NO: 205 - protein, 860 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDKGLSELSPMQQALIYSFCENHDKAIEVLDGISWNRAEMTMCALLAKVQMKAKRTKEAVEVLKKALDAISHSDKGPDATAISADCLYNLGLCYMEEGNLQMAFDSFTKAVKANPDFAESFYQRGLCKVKLHKDSSILDFNRAITLNPKHYQAYLSRVAFYGLKGRYSKAILNCNKAIKIYPESVRAYLYRGVLKYYNKTYKLAITDLTTAISMDKNSYIAFYNRALCYTKIRELQMALTDYGIVLLLDATETVKLNTFLNRGLIYVELGQYGFALEDFKQAALISRTNGSLCHATAMCHHRINEFEEAVNFFTWALKINPCFLDAYVGRGNSYMEYGHDEATKQAQKDFLKALHINPAYIKARISFGYNLQAQGKFQKAWNHFTIAIDTDPKNYLAYEGRAVVCLQMGNNFAAMQDINAAMKISTTAEFLTNRGVIHEFMGHKQNAMKDYQDAITLNPKYSLAYFNAGNIYFHHRQFSQASDYFSKALKFDPENEYVLMNRAITNTILKKYEEAKEDFANVIESCPFWAAVYFNRAHFYYCLKQYELAEEDLNKALSLKPNDALVYNFRAKVRGKIGLIEEAMADYNQALDLEDYASVI

SEQ ID NO: 206 - protein, 600 aa, Homo sapiens [sequence omitted]

SEQ ID NO: 207 - protein, 518 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLVLYGHSTQDLPETNARVVGGTEAGRNSWPSQISLQYRSGGSRYHTCGGTLIRQNWVMTAAHCVDYQKTFRVVAGDHNLSQNDGTEQYVSVQKIVVHPYWNSDNVAAGYDIALLRLAQSVTLNSYVQLGVLPQEGAILANNSPCYITGWGKTKTNGQLAQTLQQAYLPSVDYAICSSSSYWGSTVKNTMVCAGGDGVRSGCQGDSGGPLHCLVNGKYSVHGVTSFVSSRGCNVSRKPTVFTQVSAYISWINNVIASN

SEQ ID NO: 208 - protein, 258 aa, Homo sapiens
MLVLYGHSTQDLPETNARVVGGTEAGRNSWPSQISLQYRSGGSRYHTCGGTLIRQNWVMTAAHCVDYQKTFRVVAGDHNLSQNDGTEQYVSVQKIVVHPYWNSDNVAAGYDIALLRLAQSVTLNSYVQLGVLPQEGAILANNSPCYITGWGKTKTNGQLAQTLQQAYLPSVDYAICSSSSYWGSTVKNTMVCAGGDGVRSGCQGDSGGPLHCLVNGKYSVHGVTSFVSSRGCNVSRKPTVFTQVSAYISWINNVIASN

SEQ ID NO: 209 - protein, 486 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMCLFRQYATCFNSGIYLIWTLLVVVGIGSVYFHATLSFLGQMLDELAVLWVLMCALAMWFPRRYLPKIFRNDRGRFKVVVSVLSAVTTCLAFVKPAINNISLMTLGVPCTALLIAELKRCDNMRVFKLGLFSGLWWTLALFCWISDRAFCELLSSFNFPYLHCMWHILICLAAYLGCVCFAYFDAASEIPEQGPVIKFWPNEKWAFIGVPYVSLLCANKKSSVKIT

SEQ ID NO: 210 - protein, 226 aa, Homo sapiens
MCLFRQYATCFNSGIYLIWTLLVVVGIGSVYFHATLSFLGQMLDELAVLWVLMCALAMWFPRRYLPKIFRNDRGRFKVVVSVLSAVTTCLAFVKPAINNISLMTLGVPCTALLIAELKRCDNMRVFKLGLFSGLWWTLALFCWISDRAFCELLSSFNFPYLHCMWHILICLAAYLGCVCFAYFDAASEIPEQGPVIKFWPNEKWAFIGVPYVSLLCANKKSSVKIT

SEQ ID NO: 211 - protein, 581 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMATRRLTDAFLLLRNNSIQNRQLLAEQVSSHITSSPLHSRSIAALADDRMALVSGISLDPEAAIGVTKRPPPKWVDGVDEIQYDVGRIKQKMKELASLHDKHLNRPTLDDSSEEEHAIEITTQEITQLFHRCQRAVQALPSRARACSEQEGRLLGNVVASLAQALQELSTSFRHAQSGYLKRMKNREERSQHFFDTSVPLMDDGDDNTLYHRGFTEDQLVLVEQNTLMVEEREREIRQIVQSISDLNEIFRDLGAMIVEQGTVLDRIDYNVEQSCIKTEDGLKQLHKAEQYQKKNRKMLVILILFVIIIVLIVVLVGVKSR

SEQ ID NO: 212 - protein, 321 aa, Homo sapiens
MATRRLTDAFLLLRNNSIQNRQLLAEQVSSHITSSPLHSRSIAALADDRMALVSGISLDPEAAIGVTKRPPPKWVDGVDEIQYDVGRIKQKMKELASLHDKHLNRPTLDDSSEEEHAIEITTQEITQLFHRCQRAVQALPSRARACSEQEGRLLGNVVASLAQALQELSTSFRHAQSGYLKRMKNREERSQHFFDTSVPLMDDGDDNTLYHRGFTEDQLVLVEQNTLMVEEREREIRQIVQSISDLNEIFRDLGAMIVEQGTVLDRIDYNVEQSCIKTEDGLKQLHKAEQYQKKNRKMLVILILFVIIIVLIVVLVGVKSR

SEQ ID NO: 213 - protein, 496 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAWSPLLLTLLAHCTGSWAQSVLTQPPSVSGAPGQRVTISCTGSSSNIGAGYDVHWYQQLPGTAPRLLIYGNSNRPSGVPDRFSGSKSGTSASLAITGLQAEDEADYYCQSYDSSLSGVMFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 214 - protein, 236 aa, Homo sapiens [sequence omitted]

SEQ ID NO: 215 - protein, 516 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDILLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 216 - protein, 256 aa, Homo sapiens [sequence omitted]

SEQ ID NO: 217 - protein, 594 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLGIMAWNATCKNWLAAEAALEKYYLSIFYGIEFVVGVLGNTIVVYGYIFSLKNWNSSNIYLFNLSVSDLAFLCTLPMLIRSYANGNWIYGDVLCISNRYVLHANLYTSILFLTFISIDRYLIIKYPFREHLLQKKEFAILISLAIWVLVTLELLPILPLINPVITDNGTTCNDFASSGDPNYNLIYSMCLTLLGFLIPLFVMCFFYYKIALFLKQRNRQVATALPLEKPLNLVIMAVVIFSVLFTPYHVMRNVRIASRLGSWKQYQCTQVVINSFYIVTRPLAFLNSVINPVFYFLLGDHFRDMLMNQLRHNFKSLTSFSRWAHELLLSFREK

SEQ ID NO: 218 - protein, 334 aa, Homo sapiens
MLGIMAWNATCKNWLAAEAALEKYYLSIFYGIEFVVGVLGNTIVVYGYIFSLKNWNSSNIYLFNLSVSDLAFLCTLPMLIRSYANGNWIYGDVLCISNRYVLHANLYTSILFLTFISIDRYLIIKYPFREHLLQKKEFAILISLAIWVLVTLELLPILPLINPVITDNGTTCNDFASSGDPNYNLIYSMCLTLLGFLIPLFVMCFFYYKIALFLKQRNRQVATALPLEKPLNLVIMAVVIFSVLFTPYHVMRNVRIASRLGSWKQYQCTQVVINSFYIVTRPLAFLNSVINPVFYFLLGDHFRDMLMNQLRHNFKSLTSFSRWAHELLLSFREK

SEQ ID NO: 219 - protein, 418 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGIGCWRNPLLLLIALVLSAKLGHFQRWEGFQQKLMSKKNMNSTLNFFIQSYNNASNDTYLYRVQRLIRSQMQLTTGVEYIVTVKIGRTKCKRNDTSNSSCPLQSKKLRKSLICESLIYTMPWINYFQLWNNSCLEAEHVGRNLRNPAFLYKVVKASS

SEQ ID NO: 220 - protein, 145 aa, Homo sapiens [sequence omitted]

SEQ ID NO: 221 - protein, 690 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPNPAFLYKVVKASS

SEQ ID NO: 222 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 223 - protein, 690 aa, synthetic construct; identical to SEQ ID NO: 221

SEQ ID NO: 224 - protein, 417 aa, Homo sapiens; identical to SEQ ID NO: 222

SEQ ID NO: 225 - protein, 1113 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAGPGSTGGQIGAAALAGGARSKVAPSVDFDHSCSDSVEYLTLNFGPFETVHRWRRLPPCDEFVGARRSKHTVVAYKDAIYVFGGDNGKTMLNDLLRFDVKDCSWCRAFTTGTPPAPRYHHSAVVYGSSMFVFGGYTGDIYSNSNLKNKNDLFEYKFATGQWTEWKIEGRLPVARSAHGATVYSDKLWIFAGYDGNARLNDMWTIGLQDRELTCWEEVAQSGEIPPSCCNFPVAVCRDKMFVFSGQSGAKITNNLFQFEFKDKTWTRIPTEHLLRGSPPPPQRRYGHTMVAFDRHLYVFGGAADNTLPNELHCYDVDFQTWEVVQPSSDSEVGGAEVPERACASEEVPTLTYEERVGFKKSRDVFGLDFGTTSAKQPTQPASELPSGRLFHAAAVISDAMYIFGGTVDNNIRSGEMYRFQFSCYPKCTLHEDYGRLWESRQFCDVEFVLGEKEECVQGHVAIVTARSRWLRRKITQARERLAQKLEQEAAPVPREAPGVAAGGARPPLLHVAIREAEARPFEVLMQFLYTDKIKYPRKGHVEDVLLIMDVYKLALSFQLCRLEQLCRQYIEASVDLQNVLVVCESAARLQLSQLKEHCLNFVVKESHFNQVIMMKEFERLSSPLIVEIVRRKQQPPPRTPLDQPVDIGTSLIQDMKAYLEGAGAEFCDITLLLDGHPRPAHKAILAARSSYFEAMFRSFMPEDGQVNISIGEMVPSRQAFESMLRYIYYGEVNMPPEDSLYLFAAPYYYGFYNNRLQAYCKQNLEMNVTVQNVLQILEAADKTQALDMKRHCLHIIVHQFTKVSKLPTLRSLSQQLLLDIIDSLASHISDKQCAELGADINPAFLYKVVKASS

SEQ ID NO: 226 - protein, 840 aa, Homo sapiens
MAGPGSTGGQIGAAALAGGARSKVAPSVDFDHSCSDSVEYLTLNFGPFETVHRWRRLPPCDEFVGARRSKHTVVAYKDAIYVFGGDNGKTMLNDLLRFDVKDCSWCRAFTTGTPPAPRYHHSAVVYGSSMFVFGGYTGDIYSNSNLKNKNDLFEYKFATGQWTEWKIEGRLPVARSAHGATVYSDKLWIFAGYDGNARLNDMWTIGLQDRELTCWEEVAQSGEIPPSCCNFPVAVCRDKMFVFSGQSGAKITNNLFQFEFKDKTWTRIPTEHLLRGSPPPPQRRYGHTMVAFDRHLYVFGGAADNTLPNELHCYDVDFQTWEVVQPSSDSEVGGAEVPERACASEEVPTLTYEERVGFKKSRDVFGLDFGTTSAKQPTQPASELPSGRLFHAAAVISDAMYIFGGTVDNNIRSGEMYRFQFSCYPKCTLHEDYGRLWESRQFCDVEFVLGEKEECVQGHVAIVTARSRWLRRKITQARERLAQKLEQEAAPVPREAPGVAAGGARPPLLHVAIREAEARPFEVLMQFLYTDKIKYPRKGHVEDVLLIMDVYKLALSFQLCRLEQLCRQYIEASVDLQNVLVVCESAARLQLSQLKEHCLNFVVKESHFNQVIMMKEFERLSSPLIVEIVRRKQQPPPRTPLDQPVDIGTSLIQDMKAYLEGAGAEFCDITLLLDGHPRPAHKAILAARSSYFEAMFRSFMPEDGQVNISIGEMVPSRQAFESMLRYIYYGEVNMPPEDSLYLFAAPYYYGFYNNRLQAYCKQNLEMNVTVQNVLQILEAADKTQALDMKRHCLHIIVHQFTKVSKLPTLRSLSQQLLLDIIDSLASHISDKQCAELGADI

SEQ ID NO: 227 - protein, 1246 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDPFRPSFRGQSPIHPSQCQAVRMPGCWPQASKPLDPALGRGAPAGRGHVFGKPEEPSTQRGPAQRESVGLVSMFRGLGIETVSKTPLKREMLPSGRGILGRGLSANLVRKDREELSPTFWDPKVLAAGDSKMAETSVGWSRTLGRGSSDASLLPLGRAAGGISREVDKPPCTFSTPSRGPPQLSSPPALPQSPLHSPDRPLVLTVEHKEKELIVKQGSKGTPQSLGLNLVKIQCHNEAVYQYHVTFSPNVECKSMRFGMLKDHQAVTGNVTAFDGSILYLPVKLQQVLELKSQRKTDSAEISIKIQMTKILEPCSDLCIPFYNVVFRRVMKLLDMKLVGRNFYDPTSAMVLQQHRLQIWPGYAASIRRTDGGLFLLADVSHKVIRNDCVLDVMHAIYQQNKEHFQDECTKLLVGNIVITRYNNRTYRIDDVDWNKTPKDSFTMSDGKEITFLEYYSKNYGITVKEEDQPLLIHRPSERQDNHGMLLKGEILLLPELSFMTGIPEKMKKDFRAMKDLAQQINLSPKQHHSALECLLQRIAKNEAATNELMRWGLRLQKDVHKIEGRVLPMERINLKNTSFITSQELNWVKEVTRDPSILTIPMHFWALFYPKRAMDQARELVNMLEKIAGPIGMRMSPPAWVELKDDRIETYVRTIQSTLGAEGKIQMVVCIIMGPRDDLYGAIKKLCCVQSPVPSQVVNVRTIGQPTRLRSVAQKILLQINCKLGGELWGVDIPLKQLMVIGMDVYHDPSRGMRSVVGFVASINLTLTKWYSRVVFQMPHQEIVDSLKLCLVGSLKKFYEVNHCLPEKIVVYRDGVSDGQLKTVANYEIPQLQKCFEAFENYQPKMVVFVVQKKISTNLYLAAPQNFVTPTPGTVVDHTITSCEWVDFYLLAHHVRQGCGIPTHYVCVLNTANLSPDHMQRLTFKLCHMYWNWPGTIRVPAPCKYAHKLAFLSGHILHHEPAIQLCENLFFLNPAFLYKVVKASS

SEQ ID NO: 228 - protein, 973 aa, Homo sapiens
MDPFRPSFRGQSPIHPSQCQAVRMPGCWPQASKPLDPALGRGAPAGRGHVFGKPEEPSTQRGPAQRESVGLVSMFRGLGIETVSKTPLKREMLPSGRGILGRGLSANLVRKDREELSPTFWDPKVLAAGDSKMAETSVGWSRTLGRGSSDASLLPLGRAAGGISREVDKPPCTFSTPSRGPPQLSSPPALPQSPLHSPDRPLVLTVEHKEKELIVKQGSKGTPQSLGLNLVKIQCHNEAVYQYHVTFSPNVECKSMRFGMLKDHQAVTGNVTAFDGSILYLPVKLQQVLELKSQRKTDSAEISIKIQMTKILEPCSDLCIPFYNVVFRRVMKLLDMKLVGRNFYDPTSAMVLQQHRLQIWPGYAASIRRTDGGLFLLADVSHKVIRNDCVLDVMHAIYQQNKEHFQDECTKLLVGNIVITRYNNRTYRIDDVDWNKTPKDSFTMSDGKEITFLEYYSKNYGITVKEEDQPLLIHRPSERQDNHGMLLKGEILLLPELSFMTGIPEKMKKDFRAMKDLAQQINLSPKQHHSALECLLQRIAKNEAATNELMRWGLRLQKDVHKIEGRVLPMERINLKNTSFITSQELNWVKEVTRDPSILTIPMHFWALFYPKRAMDQARELVNMLEKIAGPIGMRMSPPAWVELKDDRIETYVRTIQSTLGAEGKIQMVVCIIMGPRDDLYGAIKKLCCVQSPVPSQVVNVRTIGQPTRLRSVAQKILLQINCKLGGELWGVDIPLKQLMVIGMDVYHDPSRGMRSVVGFVASINLTLTKWYSRVVFQMPHQEIVDSLKLCLVGSLKKFYEVNHCLPEKIVVYRDGVSDGQLKTVANYEIPQLQKCFEAFENYQPKMVVFVVQKKISTNLYLAAPQNFVTPTPGTVVDHTITSCEWVDFYLLAHHVRQGCGIPTHYVCVLNTANLSPDHMQRLTFKLCHMYWNWPGTIRVPAPCKYAHKLAFLSGHILHHEPAIQLCENLFFL

SEQ ID NO: 229 - protein, 1584 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSRRKQAKPRSLKDPNCKLEDKTEDGEALDCKKRPEDGEELEDEAVHSCDSCLQVFESLSDITEHKINQCQLTDGVDVEDDPTCSWPASSPSSKDQTSPSHGEGCDFGEEEGGPGLPYPCQFCDKSFSRLSYLKHHEQSHSDKLPFKCTYCSRLFKHKRSRDRHIKLHTGDKKYHCSECDAAFSRSDHLKIHLKTHTSNKPYKCAICRRGFLSSSSLHGHMQVHERNKDGSQSGSRMEDWKMKDTQKCSQCEEGFDFPEDLQKHIAECHPECSPNEDRAALQCVYCHELFVEETSLMNHMEQVHSGEKKNSCSICSESFHTVEELYSHMDSHQQPESCNHSNSPSLVTVGYTSVSSTTPDSNLSVDSSTMVEAAPPIPKSRGRKRAAQQTPDMTGPSSKQAKVTYSCIYCNKQLFSSLAVLQIHLKTMHLDKPEQAHICQYCLEVLPSLYNLNEHLKQVHEAQDPGLIVSAMPAIVYQCNFCSEVVNDLNTLQEHIRCSHGFANPAAKDSNAFFCPHCYMGFLTDSSLEEHIRQVHCDLSGSRFGSPVLGTPKEPVVEVYSCSYCTNSPIFNSVLKLNKHIKENHKNIPLALNYIHNGKKSRALSPLSPVAIEQTSLKMMQAVGGAPARPTGEYICNQCGAKYTSLDSFQTHLKTHLDTVLPKLTCPQCNKEFPNQESLLKHVTIHFMITSTYYICESCDKQFTSVDDLQKHLLDMHTFVFFRCTLCQEVFDSKVSIQLHLAVKHSNEKKVYRCTSCNWDFRNETDLQLHVKHNHLENQGKVHKCIFCGESFGTEVELQCHITTHSKKYNCKFCSKAFHAIILLEKHLREKHCVFETKTPNCGTNGASEQVQKEEVELQTLLTNSQESHNSHDGSEEDVDTSEPMYGCDICGAAYTMETLLQNHQLRDHNIRPGESAIVKKKAELIKGNYKCNVCSRTFFSENGLREHMQTHLGPVKHYMCPICGERFPSLLTLTEHKVTHSKSLDTGNCRICKMPLQSEEEFLEHCQMHPDLRNSLTGFRCVVCMQTVTSTLELKIHGTFHMQKTGNGSAVQTTGRGQHVQKLYKCASCLKEFRSKQDLVKLDINGLPYGLCAGCVNLSKSASPGINVPPGTNRPGLGQNENLSAIEGKGKVGGLKTRCSSCNVKFESESELQNHIQTIHRELVPDSNSTQLKTPQVSPMPRISPSQSDEKKTYQCIKCQMVFYNEWDIQVHVANHMIDEGLNHECKLCSQTFDSPAKLQCHLIEHSFEGMGGTFKCPVCFTVFVQANKLQQHIFSAHGQEDKIYDCTQCPQKFFFQTELQNHTMTQHSSNPAFLYKVVKASS

SEQ ID NO: 230 - protein, 1311 aa, Homo sapiens
MSRRKQAKPRSLKDPNCKLEDKTEDGEALDCKKRPEDGEELEDEAVHSCDSCLQVFESLSDITEHKINQCQLTDGVDVEDDPTCSWPASSPSSKDQTSPSHGEGCDFGEEEGGPGLPYPCQFCDKSFSRLSYLKHHEQSHSDKLPFKCTYCSRLFKHKRSRDRHIKLHTGDKKYHCSECDAAFSRSDHLKIHLKTHTSNKPYKCAICRRGFLSSSSLHGHMQVHERNKDGSQSGSRMEDWKMKDTQKCSQCEEGFDFPEDLQKHIAECHPECSPNEDRAALQCVYCHELFVEETSLMNHMEQVHSGEKKNSCSICSESFHTVEELYSHMDSHQQPESCNHSNSPSLVTVGYTSVSSTTPDSNLSVDSSTMVEAAPPIPKSRGRKRAAQQTPDMTGPSSKQAKVTYSCIYCNKQLFSSLAVLQIHLKTMHLDKPEQAHICQYCLEVLPSLYNLNEHLKQVHEAQDPGLIVSAMPAIVYQCNFCSEVVNDLNTLQEHIRCSHGFANPAAKDSNAFFCPHCYMGFLTDSSLEEHIRQVHCDLSGSRFGSPVLGTPKEPVVEVYSCSYCTNSPIFNSVLKLNKHIKENHKNIPLALNYIHNGKKSRALSPLSPVAIEQTSLKMMQAVGGAPARPTGEYICNQCGAKYTSLDSFQTHLKTHLDTVLPKLTCPQCNKEFPNQESLLKHVTIHFMITSTYYICESCDKQFTSVDDLQKHLLDMHTFVFFRCTLCQEVFDSKVSIQLHLAVKHSNEKKVYRCTSCNWDFRNETDLQLHVKHNHLENQGKVHKCIFCGESFGTEVELQCHITTHSKKYNCKFCSKAFHAIILLEKHLREKHCVFETKTPNCGTNGASEQVQKEEVELQTLLTNSQESHNSHDGSEEDVDTSEPMYGCDICGAAYTMETLLQNHQLRDHNIRPGESAIVKKKAELIKGNYKCNVCSRTFFSENGLREHMQTHLGPVKHYMCPICGERFPSLLTLTEHKVTHSKSLDTGNCRICKMPLQSEEEFLEHCQMHPDLRNSLTGFRCVVCMQTVTSTLELKIHGTFHMQKTGNGSAVQTTGRGQHVQKLYKCASCLKEFRSKQDLVKLDINGLPYGLCAGCVNLSKSASPGINVPPGTNRPGLGQNENLSAIEGKGKVGGLKTRCSSCNVKFESESELQNHIQTIHRELVPDSNSTQLKTPQVSPMPRISPSQSDEKKTYQCIKCQMVFYNEWDIQVHVANHMIDEGLNHECKLCSQTFDSPAKLQCHLIEHSFEGMGGTFKCPVCFTVFVQANKLQQHIFSAHGQEDKIYDCTQCPQKFFFQTELQNHTMTQHSS

SEQ ID NO: 231 - protein, 507 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEMGRRIHSELRNRAPSDVKELALDNSRSNEGKLEALTDEFEELEFLSKINGGLTSISDLPKLKLRKLELRVSGGLEVLAEKCPNLTHLYLSGNKIKDLSTIEPLKQLENLKSLDLFNCEVTNLNDYGENVFKLLLQLTYLDSCYWDHKEAPYSDIEDHVEGLDDEEEGEHEEEYDEDAQVVEDEEGEEEEEEGEEEDVSGGDEEDEEGYNDGEVDGEEDEEELGEEERGQKRKNPAFLYKVVKASS

SEQ ID NO: 232 - protein, 234 aa, Homo sapiens
MEMGRRIHSELRNRAPSDVKELALDNSRSNEGKLEALTDEFEELEFLSKINGGLTSISDLPKLKLRKLELRVSGGLEVLAEKCPNLTHLYLSGNKIKDLSTIEPLKQLENLKSLDLFNCEVTNLNDYGENVFKLLLQLTYLDSCYWDHKEAPYSDIEDHVEGLDDEEEGEHEEEYDEDAQVVEDEEGEEEEEEGEEEDVSGGDEEDEEGYNDGEVDGEEDEEELGEEERGQKRK

SEQ ID NO: 233 - protein, 544 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSPLLGLRSELQDTCTSLGLMLSVVLLMGLARVVARQQLHRPVAHAFVLEFLATFQLCCCTHELQLLSEQHPAHPTWTLTLVYFFSLVHGLTLVGTSSNPCGVMMQMMLGGMSPETGAVRLLAQLVSALCSRYCTSALWSLGLTQYHVSERSFACKNPIRVDLLKAVITEAVCSFLFHSALLHFQEVRTKLRIHLLAALITFLVYAGGSLTGAVFNPALALSLHFMCFDEAFPQFFIVYWLAPSLGILLMILMFSFFLPWLHNNHTINKKENPAFLYKVVKASS

SEQ ID NO: 234 - protein, 271 aa, Homo sapiens
MSPLLGLRSELQDTCTSLGLMLSVVLLMGLARVVARQQLHRPVAHAFVLEFLATFQLCCCTHELQLLSEQHPAHPTWTLTLVYFFSLVHGLTLVGTSSNPCGVMMQMMLGGMSPETGAVRLLAQLVSALCSRYCTSALWSLGLTQYHVSERSFACKNPIRVDLLKAVITEAVCSFLFHSALLHFQEVRTKLRIHLLAALITFLVYAGGSLTGAVFNPALALSLHFMCFDEAFPQFFIVYWLAPSLGILLMILMFSFFLPWLHNNHTINKKE

SEQ ID NO: 235 - protein, 613 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEALAMGSRALRLWLVAPGGGIKWRFIATSSASQLSPTELTEMRNDLFNKEKARQLSLTPRTEKIEVKHVGKTDPGTVFVMNKNISTPYSCAMHLSEWYCRKSILALVDGQPWDMYKPLTKSCEIKFLTFKDCDPGEVNKAYWRSCAMMMGCVIERAFKDEYMVNLVRAPEVPVISGAFCYDVVLDSKLDEWMPTKENLRSFTKDAHALIYKDLPFETLEVEAKVALEIFQHSKYKVDFIEEKASQNPERIVKLHRIGDFIDVSEGPLIPRTSICFQYEVSAVHNLQPTQPSLIRRFQGVSLPVHLRAHFTIWDKLLERSRKMTPFPILLLFTTQSFFTTSPESYLLHGTVSE

SEQ ID NO: 236 - protein, 353 aa, Homo sapiens
MEALAMGSRALRLWLVAPGGGIKWRFIATSSASQLSPTELTEMRNDLFNKEKARQLSLTPRTEKIEVKHVGKTDPGTVFVMNKNISTPYSCAMHLSEWYCRKSILALVDGQPWDMYKPLTKSCEIKFLTFKDCDPGEVNKAYWRSCAMMMGCVIERAFKDEYMVNLVRAPEVPVISGAFCYDVVLDSKLDEWMPTKENLRSFTKDAHALIYKDLPFETLEVEAKVALEIFQHSKYKVDFIEEKASQNPERIVKLHRIGDFIDVSEGPLIPRTSICFQYEVSAVHNLQPTQPSLIRRFQGVSLPVHLRAHFTIWDKLLERSRKMTPFPILLLFTTQSFFTTSPESYLLHGTVSE

SEQ ID NO: 237 - protein, 677 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQNPAFLYKVVKASS

SEQ ID NO: 238 - protein, 404 aa, Homo sapiens
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 239 - protein, 1100 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVSVFRSEEMCLSQLFLQVEAAYCCVAELGELGLVQFKDLNMNVNSFQRKFVNEVRRCESLERILRFLEDEMQNEIVVQLLEKSPLTPLPREMITLETVLEKLEGELQEANQNQQALKQSFLELTELKYLLKKTQDFFETETNLADDFFTEDTSGLLELKAVPAYMTGKLGFIAGVINRERMASFERLLWRICRGNVYLKFSEMDAPLEDPVTKEEIQKNIFIIFYQGEQLRQKIKKICDGFRATVYPCPERAVERREMLESVNVRLEDLITVITQTESHRQRLLQEAAANWHSWLIKVQKMKAVYHILNMCNIDVTQQCVIAEIWFPVADATRIKRALEQGMELSGSSMAPIMTTVQSKTAPPTFNRTNKFTAGFQNIVDAYGVGSYREINPAPYTIITFPFLFAVMFGDCGHGTVMLLAALWMILNERRLLSQKTDNEIWNTFFHGRYLILLMGIFSIYTGLIYNDCFSKSLNIFGSSWSVQPMFRNGTWNTHVMEESLYLQLDPAIPGVYFGNPYPFGIDPIWNLASNKLTFLNSYKMKMSVILGIVQMVFGVILSLFNHIYFRRTLNIILQFIPEMIFILCLFGYLVFMIIFKWCCFDVHVSQHAPSILIHFINMFLFNYSDSSNAPLYKHQQEVQSFFVVMALISVPWMLLIKPFILRASHRKSQLQASRIQEDATENIEGDSSSPSSRSGQRTSADTHGALDDHGEEFNFGDVFVHQAIHTIEYCLGCISNTASYLRLWALSLAHAQLSEVLWTMVMNSGLQTRGWGGIVGVFIIFAVFAVLTVAILLIMEGLSAFLHALRLHWVEFQNKFYVGDGYKFSPFSFKHILDGTAEE

SEQ ID NO: 240 - protein, 840 aa, Homo sapiens
MVSVFRSEEMCLSQLFLQVEAAYCCVAELGELGLVQFKDLNMNVNSFQRKFVNEVRRCESLERILRFLEDEMQNEIVVQLLEKSPLTPLPREMITLETVLEKLEGELQEANQNQQALKQSFLELTELKYLLKKTQDFFETETNLADDFFTEDTSGLLELKAVPAYMTGKLGFIAGVINRERMASFERLLWRICRGNVYLKFSEMDAPLEDPVTKEEIQKNIFIIFYQGEQLRQKIKKICDGFRATVYPCPERAVERREMLESVNVRLEDLITVITQTESHRQRLLQEAAANWHSWLIKVQKMKAVYHILNMCNIDVTQQCVIAEIWFPVADATRIKRALEQGMELSGSSMAPIMTTVQSKTAPPTFNRTNKFTAGFQNIVDAYGVGSYREINPAPYTIITFPFLFAVMFGDCGHGTVMLLAALWMILNERRLLSQKTDNEIWNTFFHGRYLILLMGIFSIYTGLIYNDCFSKSLNIFGSSWSVQPMFRNGTWNTHVMEESLYLQLDPAIPGVYFGNPYPFGIDPIWNLASNKLTFLNSYKMKMSVILGIVQMVFGVILSLFNHIYFRRTLNIILQFIPEMIFILCLFGYLVFMIIFKWCCFDVHVSQHAPSILIHFINMFLFNYSDSSNAPLYKHQQEVQSFFVVMALISVPWMLLIKPFILRASHRKSQLQASRIQEDATENIEGDSSSPSSRSGQRTSADTHGALDDHGEEFNFGDVFVHQAIHTIEYCLGCISNTASYLRLWALSLAHAQLSEVLWTMVMNSGLQTRGWGGIVGVFIIFAVFAVLTVAILLIMEGLSAFLHALRLHWVEFQNKFYVGDGYKFSPFSFKHILDGTAEE

SEQ ID NO: 241 - protein, 890 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAGVAAWLPFARAAAIGWMPVASGPMPAPPRQERKRTQDALIVLNVSGTRFQTWQDTLERYPDTLLGSSERDFFYHPETQQYFFDRDPDIFRHILNFYRTGKLHYPRHECISAYDEELAFFGLIPEIIGDCCYEEYKDRRRENAERLQDDADTDTAGESALPTMTARQRVWRAFENPHTSTMALVFYYVTGFFIAVSVIANVVETVPCGSSPGHIKELPCGERYAVAFFCLDTACVMIFTVEYLLRLAAAPSRYRFVRSVMSIIDVVAILPYYIGLVMTDNEDVSGAFVTLRVFRVFRIFKFSRHSQGLRILGYTLKSCASELGFLLFSLTMAIIIFATVMFYAEKGSSASKFTSIPAAFWYTIVTMTTLGYGDMVPKTIAGKIFGSICSLSGVLVIALPVPVIVSNFSRIYHQNQRADKRRAQKKARLARIRAAKSGSANAYMQSKRNGLLSNQLQSSEDEQAFVSKSGSSFETQHHHLLHCLEKTTNHEFVDEQVFEESCMEVATVNRPSSHSPSLSSQQGVTSTCCSRRHKKTFRIPNANVSGSHQGSIQELSTIQIRCVERTPLSNSRSSLNAKMEECVKLNCEQPYVTTAIISIPTPPVTTPEGDDRPESPEYSGGNIVRVSAL

SEQ ID NO: 242 - protein, 630 aa, Homo sapiens
MAAGVAAWLPFARAAAIGWMPVASGPMPAPPRQERKRTQDALIVLNVSGTRFQTWQDTLERYPDTLLGSSERDFFYHPETQQYFFDRDPDIFRHILNFYRTGKLHYPRHECISAYDEELAFFGLIPEIIGDCCYEEYKDRRRENAERLQDDADTDTAGESALPTMTARQRVWRAFENPHTSTMALVFYYVTGFFIAVSVIANVVETVPCGSSPGHIKELPCGERYAVAFFCLDTACVMIFTVEYLLRLAAAPSRYRFVRSVMSIIDVVAILPYYIGLVMTDNEDVSGAFVTLRVFRVFRIFKFSRHSQGLRILGYTLKSCASELGFLLFSLTMAIIIFATVMFYAEKGSSASKFTSIPAAFWYTIVTMTTLGYGDMVPKTIAGKIFGSICSLSGVLVIALPVPVIVSNFSRIYHQNQRADKRRAQKKARLARIRAAKSGSANAYMQSKRNGLLSNQLQSSEDEQAFVSKSGSSFETQHHHLLHCLEKTTNHEFVDEQVFEESCMEVATVNRPSSHSPSLSSQQGVTSTCCSRRHKKTFRIPNANVSGSHQGSIQELSTIQIRCVERTPLSNSRSSLNAKMEECVKLNCEQPYVTTAIISIPTPPVTTPEGDDRPESPEYSGGNIVRVSAL

SEQ ID NO: 243 - protein, 586 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMCPLTLQVTGLMNVSEPNSSFAFVNEFILQGFTCEWTIQIFLFSLFTTTYALTITGNGAIAFVLWCDWRLHTPMYMFLGNFSFLEIWYVSSTVPKMLVNFLSEKKNISFAGCFLQFYFFFSLGTSECLLLTVMAFDQYLAICRPLLYPNIMTGHLCAKLVILCWVCGFLWFLIPIVLISQMPFCGPNIIDHVVCDPGPRFALDCVSAPRIQLFCYTLSSLVIFGNFLFIIGSYTLVLKAVLGMPSSTGRHKAFSTCGSHLAVVSLCYSSLMVMYVSPGLGHSTGMQKIETLFYAMVTPLFNPLIYSLQNKEIKAALRKVLGSSNII

SEQ ID NO: 244 - protein, 326 aa, Homo sapiens
MCPLTLQVTGLMNVSEPNSSFAFVNEFILQGFTCEWTIQIFLFSLFTTTYALTITGNGAIAFVLWCDWRLHTPMYMFLGNFSFLEIWYVSSTVPKMLVNFLSEKKNISFAGCFLQFYFFFSLGTSECLLLTVMAFDQYLAICRPLLYPNIMTGHLCAKLVILCWVCGFLWFLIPIVLISQMPFCGPNIIDHVVCDPGPRFALDCVSAPRIQLFCYTLSSLVIFGNFLFIIGSYTLVLKAVLGMPSSTGRHKAFSTCGSHLAVVSLCYSSLMVMYVSPGLGHSTGMQKIETLFYAMVTPLFNPLIYSLQNKEIKAALRKVLGSSNII

SEQ ID NO: 245 - protein, 307 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKKRSVSGCNITIFAVMFSHLSAGKSPCGNQANVLCISRLEFVQYQS

SEQ ID NO: 246 - protein, 47 aa, Homo sapiens
MKKRSVSGCNITIFAVMFSHLSAGKSPCGNQANVLCISRLEFVQYQS

SEQ ID NO: 247 - protein, 557 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAKVSELYDVTWEEMRDKMRKWREENSRNSEQIVEVGEELINEYASKLGDDIWIIYEQVMIAALDYGRDDLALFCLQELRRQFPGSHRVKRLTGMRFEAMERYDDAIQLYDRILQEDPTNTAARKRKIAIRKAQGKNVEAIRELSEYLEQFVGDQEAWHELAELYINEHDYAKAAFCLEELMMTNPHNHLYCQQYAEVKYTQGGLENLELSRKYFAQALKLNNRNMRALFGLYMSASHIASNPKASAKTKKDNMKYASWAASQINRAYQFAGRSKKETKYSLKAVEDMLETLQITQS

SEQ ID NO: 248 - protein, 297 aa, Homo sapiens
MAKVSELYDVTWEEMRDKMRKWREENSRNSEQIVEVGEELINEYASKLGDDIWIIYEQVMIAALDYGRDDLALFCLQELRRQFPGSHRVKRLTGMRFEAMERYDDAIQLYDRILQEDPTNTAARKRKIAIRKAQGKNVEAIRELSEYLEQFVGDQEAWHELAELYINEHDYAKAAFCLEELMMTNPHNHLYCQQYAEVKYTQGGLENLELSRKYFAQALKLNNRNMRALFGLYMSASHIASNPKASAKTKKDNMKYASWAASQINRAYQFAGRSKKETKYSLKAVEDMLETLQITQS

SEQ ID NO: 249 - protein, 385 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSSTLGKLSNQVEETLPLLKKVPANYFHICSAILMGFSLSKSATQVSAIHMDSKVDDHLIRGTEKSRLEPATQLFQNTKKIRLEDTNQENFTRIEGTGTGSLSGKALGSVVYVKESDGLEMTDVE

SEQ ID NO: 250 - protein, 125 aa, Homo sapiens
MSSTLGKLSNQVEETLPLLKKVPANYFHICSAILMGFSLSKSATQVSAIHMDSKVDDHLIRGTEKSRLEPATQLFQNTKKIRLEDTNQENFTRIEGTGTGSLSGKALGSVVYVKESDGLEMTDVE

SEQ ID NO: 251 - protein, 372 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEKILILLLVALSVAYAAPGPRGIIINLENGELCMNSAQCKSNCCQHSSALGLARCTSMASENSECSVKTLYGIYYKCPCERGLTCEGDKTIVGSITNTNFGICHDAGRSKQ

SEQ ID NO: 252 - protein, 112 aa, Homo sapiens
MEKILILLLVALSVAYAAPGPRGIIINLENGELCMNSAQCKSNCCQHSSALGLARCTSMASENSECSVKTLYGIYYKCPCERGLTCEGDKTIVGSITNTNFGICHDAGRSKQ

SEQ ID NO: 253 - protein, 543 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAGAVSLLGVVGLLLVSALSGVLGDRANPDLRAHPGNAAHPGSGATEPRRRPPLKDQRERTRAGSLPLGALYTAAVAAFVLYKCLQGKDETAVLHEEASKQQPLQSEQQLAQLTQQLAQTEQHLNNLMAQLDPLFERVTTLAGAQQELLNMKLWTIHELLQDSKPDKDMEASEPGEGSGGESAGGGDKVSETGTFLISPHTEASRPLPEDFCLKEDEEEVGDSQAWEEPTNWSTETWNLATSWEVGRGLRRRCSQAVAKGPSHSLGWEGGTTAEGRLKQSLFS

SEQ ID NO: 254 - protein, 283 aa, Homo sapiens
MAGAVSLLGVVGLLLVSALSGVLGDRANPDLRAHPGNAAHPGSGATEPRRRPPLKDQRERTRAGSLPLGALYTAAVAAFVLYKCLQGKDETAVLHEEASKQQPLQSEQQLAQLTQQLAQTEQHLNNLMAQLDPLFERVTTLAGAQQELLNMKLWTIHELLQDSKPDKDMEASEPGEGSGGESAGGGDKVSETGTFLISPHTEASRPLPEDFCLKEDEEEVGDSQAWEEPTNWSTETWNLATSWEVGRGLRRRCSQAVAKGPSHSLGWEGGTTAEGRLKQSLFS

SEQ ID NO: 255 - protein, 366 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMFAPAVMRAFRKNKTLGYGVPMLLLIVGGSFGLREFSQIRYDAVKSKMDPELEKKLKENKISLESEYEKIKDSKFDDWKNIRGPRPWEDPDLLQGRNPESLKTKTT

SEQ ID NO: 256 - protein, 106 aa, Homo sapiens
MFAPAVMRAFRKNKTLGYGVPMLLLIVGGSFGLREFSQIRYDAVKSKMDPELEKKLKENKISLESEYEKIKDSKFDDWKNIRGPRPWEDPDLLQGRNPESLKTKTT

SEQ ID NO: 257 - protein, 512 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLYAATRATLKKEFGGGHIKDEVFGTVKEDVSLHGYKKYLLSQSSPAPLTAAEEELRQIKINEVQTDVGVDTKHQTLQGVAFPISREAFQALEKLNNRQLNYVQLEIDIKNEIIILANTTNTELKDLPKRIPKDSARYHFFLYKHSHEGDYLESIVFIYSMPGYTCSIRERMLYSSCKSRLLEIVERQLQMDVIRKIEIDNGDELTADFLYEEVHPKQHAHKQSFAKPKGPAGKRGIRRLIRGPAETEATTD

SEQ ID NO: 258 - protein, 252 aa, Homo sapiens
MLYAATRATLKKEFGGGHIKDEVFGTVKEDVSLHGYKKYLLSQSSPAPLTAAEEELRQIKINEVQTDVGVDTKHQTLQGVAFPISREAFQALEKLNNRQLNYVQLEIDIKNEIIILANTTNTELKDLPKRIPKDSARYHFFLYKHSHEGDYLESIVFIYSMPGYTCSIRERMLYSSCKSRLLEIVERQLQMDVIRKIEIDNGDELTADFLYEEVHPKQHAHKQSFAKPKGPAGKRGIRRLIRGPAETEATTD

SEQ ID NO: 259 - protein, 903 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGLLPKLGASQGSDTSTSRAGRCARSVFGNIKVFVLCQGLLQLCQLLYSAYFKSSLTTIEKRFGLSSSSSGLISSLNEISNAILIIFVSYFGSRVHRPRLIGIGGLFLAAGAFILTLPHFLSEPYQYTLASTGNNSRLQAELCQKHWQDLPPSKCHSTTQNPQKETSSMWGLMVVAQLLAGIGTVPIQPFGISYVDDFSEPSNSPLYISILFAISVFGPAFGYLLGSVMLQIFVDYGRVNTAAVNLVPGDPRWIGAWWLGLLISSALLVLTSFPFFFFPRAMPIGAKRAPATADEARKLEEAKSRGSLVDFIKRFPCIFLRLLMNSLFVLVVLAQCTFSSVIAGLSTFLNKFLEKQYGTSAAYANFLIGAVNLPAAALGMLFGGILMKRFVFSLQAIPRIATTIITISMILCVPLFFMGCSTPTVAEVYPPSTSSSIHPQSPACRRDCSCPDSIFHPVCGDNGIEYLSPCHAGCSNINMSSATSKQLIYLNCSCVTGGSASAKTGSCPVPCAHFLLPAIFLISFVSLIACISHNPLYMMVLRVVNQEEKSFAIGVQFLLMRLLAWLPSPALYGLTIDHSCIRWNSLCLGRRGACAYYDNDALRDRYLGLQMGYKALGMLLLCFISWRVKKNKEYNVQKAAGLI

SEQ ID NO: 260 - protein, 643 aa, Homo sapiens [sequence omitted]

SEQ ID NO: 261 - protein, 851 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVPVAVTAAVAPVLSINSDFSDLREIKKQLLLIAGLTRERGLLHSSKWSAELAFSLPALPLAELQPPPPITEEDAQDMDAYTLAKAYFDVKEYDRAAHFLHGCNSKKAYFLYMYSRYLSGEKKKDDETVDSLGPLEKGQVKNEALRELRVELSKKHQARELDGFGLYLYGVVLRKLDLVKEAIDVFVEATHVLPLHWGAWLELCNLITDKEMLKFLSLPDTWMKEFFLAHIYTELQLIEEALQKYQNLIDVGFSKSSYIVSQIAVAYHNIRDIDKALSIFNELRKQDPYRIENMDTFSNLLYVRSMKSELSYLAHNLCEIDKYRVETCCVIGNYYSLRSQHEKAALYFQRALKLNPRYLGAWTLMGHEYMEMKNTSAAIQAYRHAIEVNKRDYRAWYGLGQTYEILKMPFYCLYYYRRAHQLRPNDSRMLVALGECYEKLNQLVEAKKCYWRAYAVGDVEKMALVKLAKLHEQLTESEQAAQCYIKYIQDIYSCGEIVEHLEESTAFRYLAQYYFKCKLWDEASTCAQKCCAFNDTREEGKALLRQILQLRNQGETPTTEVPAPFFLPASLSANNTPTRRVSPLNLSSVTP

SEQ ID NO: 262 - protein, 591 aa, Homo sapiens
MVPVAVTAAVAPVLSINSDFSDLREIKKQLLLIAGLTRERGLLHSSKWSAELAFSLPALPLAELQPPPPITEEDAQDMDAYTLAKAYFDVKEYDRAAHFLHGCNSKKAYFLYMYSRYLSGEKKKDDETVDSLGPLEKGQVKNEALRELRVELSKKHQARELDGFGLYLYGVVLRKLDLVKEAIDVFVEATHVLPLHWGAWLELCNLITDKEMLKFLSLPDTWMKEFFLAHIYTELQLIEEALQKYQNLIDVGFSKSSYIVSQIAVAYHNIRDIDKALSIFNELRKQDPYRIENMDTFSNLLYVRSMKSELSYLAHNLCEIDKYRVETCCVIGNYYSLRSQHEKAALYFQRALKLNPRYLGAWTLMGHEYMEMKNTSAAIQAYRHAIEVNKRDYRAWYGLGQTYEILKMPFYCLYYYRRAHQLRPNDSRMLVALGECYEKLNQLVEAKKCYWRAYAVGDVEKMALVKLAKLHEQLTESEQAAQCYIKYIQDIYSCGEIVEHLEESTAFRYLAQYYFKCKLWDEASTCAQKCCAFNDTREEGKALLRQILQLRNQGETPTTEVPAPFFLPASLSANNTPTRRVSPLNLSSVTP

SEQ ID NO: 263 - protein, 396 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 264 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 265 - protein, 396 aa, synthetic construct; identical to SEQ ID NO: 263

SEQ ID NO: 266 - protein, 136 aa, Homo sapiens; identical to SEQ ID NO: 264

SEQ ID NO: 267 - protein, 343 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMHSTPANPVEAVVQRAGFVASQASLRPWKQDRRAGDPWPRTHEFGYPEASLQASACKKKKSKDKKRKREDEETQLDTLESGEQ

SEQ ID NO: 268 - protein, 83 aa, Homo sapiens
MHSTPANPVEAVVQRAGFVASQASLRPWKQDRRAGDPWPRTHEFGYPEASLQASACKKKKSKDKKRKREDEETQLDTLESGEQ

SEQ ID NO: 269 - protein, 741 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMYDAERGWSLSFAGCGFLGFYHVGATRCLSEHAPHLLRDARMLFGASAGALHCVGVLSGIPLEQTLQVLSDLVRKARSRNIGIFHPSFNLSKFLRQGLGKCLPANVHQLISGKIGISLTRVSDGENVLVSDFRSKDEVVDALVCSCFMPFYSGLIPPSFRGVRYVDGGVSDNVPFIDAKTTITVSPFYGEYDICPKVKSTNFLHVDITKLSLRLCTGNLYLLSRAFVPPDLKVLGEICLRGYLDAFRFLEEKGICNRPQPGLKSSSEGMDPEVAMPSWANMSLDSSPESAALAVRLEGDELLDHLRLSILPWDESILDTLSPRLATALSEEMKDKGGYMSKICNLLPIRIMSYVMLPCTLPVESAIAIVQRLVTWLPDMPDDVLWLQWVTSQVFTRVLMCLLPASRSQMPVSSQQASPCTPEQDWPCWTPCSPEGCPAETKAEATPRSILRSSLNFFLGNKVPAGAEGLSTFPSFSLEKSL

SEQ ID NO: 270 - protein, 481 aa, Homo sapiens [sequence omitted]

SEQ ID NO: 271 - protein, 737 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 272 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 273 - protein, 356 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTPCLESFCQRAKDCKQNIPLPFLIQDWPLSESKPQGQDFWTPQPLQAAAPSHEFQQVALHPARTRLASRPQGGRLMSSREVGLRAPRCSCGKRKR

SEQ ID NO: 274 - protein, 96 aa, Homo sapiens
MTPCLESFCQRAKDCKQNIPLPFLIQDWPLSESKPQGQDFWTPQPLQAAAPSHEFQQVALHPARTRLASRPQGGRLMSSREVGLRAPRCSCGKRKR

SEQ ID NO: 275 - protein, 504 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 276 - protein, 244 aa, Homo sapiens
MDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 277 - protein, 592 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEWTAARARPLVMTLNSLGFSFGHGLTAAVAYGVRDWTLLQLVVSVPFFLCFLYSWWLAESARWLLTTGRLDWGLQELWRVAAINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQF

SEQ ID NO: 278 - protein, 332 aa, Homo sapiens
MEWTAARARPLVMTLNSLGFSFGHGLTAAVAYGVRDWTLLQLVVSVPFFLCFLYSWWLAESARWLLTTGRLDWGLQELWRVAAINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQF

SEQ ID NO: 279 - protein, 876 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMALLGSRAELEADEDVFEDALETISISSHSDMATSSLHFASCDTQQAPRQRGASTVSSSSSTKVDLKSGLEECAVALNLFLSNKFTDALELLRPWAKESMYHALGYSTIVVLQAVLTFEQQDIQNGISAMKDALQTCQKYRKKYTVVESFSSLLSRGSLEQLSEEEMHAEICYAECLLQKAALTFVQDENMINFIKGGLKIRTSYQIYKECLSILHEIQKNKLQQEFFYEFEGGVKLGSGAFNLMLSLLPARIIRLLEFIGFSGNRELGLLQLREGASGRSMRSALCCLTILAFHTYISLILGTGEVNVAEAERLLAPFLQQFPNGSLVLFYHARIELLKGNLEEAQEVFQKCVSVQEEWKQFHHLCYWELMWINVFQQNWMQAYYYSDLLCKESKWSKATYVFLKAAILSMLPEEDVVATNENVVTLFRQVDSLKQRIAGKSIPTEKFAVRKARRYSASLPAPVKLILPALEMMYVWNGFSIVSKRKDLSENLLVTVEKAEAALQSQNFNSFSVDDECLVKLLKGCCLKNLQRPLQAELCYNHVVESEKLLKYDHYLVPFTLFELASLYKSQGEIDKAIKFLETARNNYKDYSLESRLHFRIQAALHLWRKPSSD

SEQ ID NO: 280 - protein, 616 aa, Homo sapiens
MALLGSRAELEADEDVFEDALETISISSHSDMATSSLHFASCDTQQAPRQRGASTVSSSSSTKVDLKSGLEECAVALNLFLSNKFTDALELLRPWAKESMYHALGYSTIVVLQAVLTFEQQDIQNGISAMKDALQTCQKYRKKYTVVESFSSLLSRGSLEQLSEEEMHAEICYAECLLQKAALTFVQDENMINFIKGGLKIRTSYQIYKECLSILHEIQKNKLQQEFFYEFEGGVKLGSGAFNLMLSLLPARIIRLLEFIGFSGNRELGLLQLREGASGRSMRSALCCLTILAFHTYISLILGTGEVNVAEAERLLAPFLQQFPNGSLVLFYHARIELLKGNLEEAQEVFQKCVSVQEEWKQFHHLCYWELMWINVFQQNWMQAYYYSDLLCKESKWSKATYVFLKAAILSMLPEEDVVATNENVVTLFRQVDSLKQRIAGKSIPTEKFAVRKARRYSASLPAPVKLILPALEMMYVWNGFSIVSKRKDLSENLLVTVEKAEAALQSQNFNSFSVDDECLVKLLKGCCLKNLQRPLQAELCYNHVVESEKLLKYDHYLVPFTLFELASLYKSQGEIDKAIKFLETARNNYKDYSLESRLHFRIQAALHLWRKPSSD

SEQ ID NO: 281 - protein, 986 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEGERKNNNKRWYFTREQLENSPSRRFGVDPDKELSYRQQAANLLQDMGQRLNVSQLTINTAIVYMHRFYMIQSFTQFPGNSVAPAALFLAAKVEEQPKKLEHVIKVAHTCLHPQESLPDTRSEAYLQQVQDLVILESIILQTLGFELTIDHPHTHVVKCTQLVRASKDLAQTSYFMATNSLHLTTFSLQYTPPVVACVCIHLACKWSNWEIPVSTDGKHWWEYVDATVTLELLDELTHEFLQILEKTPNRLKRIWNWRACEAAKKTKADDRGTDEKTSEQTILNMISQSSSDTTIAGLMSMSTSTTSAVPSLPVSEESSSNLTSVEMLPGKRWLSSQPSFKLEPTQGHRTSENLALTGVDHSLPQDGSNAFISQKQNSKSVPSAKVSLKEYRAKHAEELAAQKRQLENMEANVKSQYAYAAQNLLSHHDSHSSVILKMPIEGSENPERPFLEKADKTALKMRIPVAGGDKAASSKPEEIKMRIKVHAAADKHNSVEDSVTKSREHKEKHKTHPSNHHHHHNHHSHKHSHSQLPVGTGNKRPGDPKHSSQTSNLAHKTYSLSSSFSSSSSTRKRGPSEETGGAVFDHPAKIAKSTKSSSLNFSFPSLPTMGQMPGHSSDTSGLSFSQPSCKTRVPHSKLDKGPTGANGHNTTQTIDYQDTVNMLHSLLSAQGVQPTQPTAFEFVRPYSDYLNPRSGGISSRSGNTDKPRPPPLPSEPPPPLPPLPK

SEQ ID NO: 282 - protein, 726 aa, Homo sapiens
MEGERKNNNKRWYFTREQLENSPSRRFGVDPDKELSYRQQAANLLQDMGQRLNVSQLTINTAIVYMHRFYMIQSFTQFPGNSVAPAALFLAAKVEEQPKKLEHVIKVAHTCLHPQESLPDTRSEAYLQQVQDLVILESIILQTLGFELTIDHPHTHVVKCTQLVRASKDLAQTSYFMATNSLHLTTFSLQYTPPVVACVCIHLACKWSNWEIPVSTDGKHWWEYVDATVTLELLDELTHEFLQILEKTPNRLKRIWNWRACEAAKKTKADDRGTDEKTSEQTILNMISQSSSDTTIAGLMSMSTSTTSAVPSLPVSEESSSNLTSVEMLPGKRWLSSQPSFKLEPTQGHRTSENLALTGVDHSLPQDGSNAFISQKQNSKSVPSAKVSLKEYRAKHAEELAAQKRQLENMEANVKSQYAYAAQNLLSHHDSHSSVILKMPIEGSENPERPFLEKADKTALKMRIPVAGGDKAASSKPEEIKMRIKVHAAADKHNSVEDSVTKSREHKEKHKTHPSNHHHHHNHHSHKHSHSQLPVGTGNKRPGDPKHSSQTSNLAHKTYSLSSSFSSSSSTRKRGPSEETGGAVFDHPAKIAKSTKSSSLNFSFPSLPTMGQMPGHSSDTSGLSFSQPSCKTRVPHSKLDKGPTGANGHNTTQTIDYQDTVNMLHSLLSAQGVQPTQPTAFEFVRPYSDYLNPRSGGISSRSGNTDKPRPPPLPSEPPPPLPPLPK

SEQ ID NO: 283 - protein, 527 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAATFFGEVVKAPCRAGTEDEEEEEEGRRETPEDREVRLQLARKREVRLLRRQTKTSLEVSLLEKYPCSKFIIAIGNNAVAFLSSFVMNSGVWEEVGCAKLWNEWCRTTDTTHLSSTEAFCVFYHLKSNPSVFGSCPRKNMQITILTCRHVTDYKTSESTGSLPSPFLRALKTQNFKDSACCPLLEQPNIVHDLPAAVLSYCQVWKIPAILYLCYTDVMKLDLITVEAFKPILSTRSLKGLVKNIPQSTEILKKLMTTNEIQSNIYT

SEQ ID NO: 284 - protein, 267 aa, Homo sapiens
MAATFFGEVVKAPCRAGTEDEEEEEEGRRETPEDREVRLQLARKREVRLLRRQTKTSLEVSLLEKYPCSKFIIAIGNNAVAFLSSFVMNSGVWEEVGCAKLWNEWCRTTDTTHLSSTEAFCVFYHLKSNPSVFGSCPRKNMQITILTCRHVTDYKTSESTGSLPSPFLRALKTQNFKDSACCPLLEQPNIVHDLPAAVLSYCQVWKIPAILYLCYTDVMKLDLITVEAFKPILSTRSLKGLVKNIPQSTEILKKLMTTNEIQSNIYT

SEQ ID NO: 285 - protein, 669 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEEGGSTGSAGSDSSTSGSGGAQQRELERMAEVLVTGEQLRLRLHEEKVIKDRRHHLKTYPNCFVAKELIDWLIEHKEASDRETAIKLMQKLADRGIIHHVCDEHKEFKDVKLFYRFRKDDGTFPLDNEVKAFMRGQRLYEKLMSPENTLLQPREEEGVKYERTFMASEFLDWLVQEGEATTRKEAEQLCHRLMEHGIIQHVSSKHPFVDSNLLYQFRMNFRRRRRLMELLNEKSPSSQETHDSPFCLRKQSHDNRKSTSFMSVSPSKEIKIVSAVRRSSMSSCGSSGYFSSSPTLSSSPPVLCNPKSVLKRPVTSEELLTPGAPYARKTFTIVGDAVGWGFVVRGSKPCHIQAVDPSGPAAAAGMKVCQFVVSVNGLNVLHVDYRTVNNLILTGPRTIVMEVMEELEC

SEQ ID NO: 286 - protein, 409 aa, Homo sapiens
MEEGGSTGSAGSDSSTSGSGGAQQRELERMAEVLVTGEQLRLRLHEEKVIKDRRHHLKTYPNCFVAKELIDWLIEHKEASDRETAIKLMQKLADRGIIHHVCDEHKEFKDVKLFYRFRKDDGTFPLDNEVKAFMRGQRLYEKLMSPENTLLQPREEEGVKYERTFMASEFLDWLVQEGEATTRKEAEQLCHRLMEHGIIQHVSSKHPFVDSNLLYQFRMNFRRRRRLMELLNEKSPSSQETHDSPFCLRKQSHDNRKSTSFMSVSPSKEIKIVSAVRRSSMSSCGSSGYFSSSPTLSSSPPVLCNPKSVLKRPVTSEELLTPGAPYARKTFTIVGDAVGWGFVVRGSKPCHIQAVDPSGPAAAAGMKVCQFVVSVNGLNVLHVDYRTVNNLILTGPRTIVMEVMEELEC

SEQ ID NO: 287 - protein, 351 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNIASATIALVGTAFLSLNIAVNIQSLRSCHSSSESPDLCNYMGSISNGMVSLLLILTLLELCVTISTIAMWCNANCCNSREEISSPPNSV

SEQ ID NO: 288 - protein, 91 aa, Homo sapiens
MNIASATIALVGTAFLSLNIAVNIQSLRSCHSSSESPDLCNYMGSISNGMVSLLLILTLLELCVTISTIAMWCNANCCNSREEISSPPNSV

SEQ ID NO: 289 - protein, 395 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPGRWRWQRDMHPARKLLSLLFLILMGTELTQVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKG

SEQ ID NO: 290 - protein, 135 aa, Homo sapiens
MPGRWRWQRDMHPARKLLSLLFLILMGTELTQVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKG

SEQ ID NO: 291 - protein, 505 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKHTQSGQSTSPLVIDFTCRVCQMAFVFSSLIPLLLMTPVFCLGNTSECFQNFSQSHKCILMHSPPSAMAELPPSANTSVCSTLYFYGIAIFLGSFVLSLLTIMVLLIRAQTLYKKFVKSTGFLGSEQWAVIHIVDQRVRFYPVAFFCCWGPAVILMIIKLTKPQDTKLRMALYVLQALTATSQGLLNCGVYGWTQHKFHQLKQEARRDADTQTPLLCSQKRFYSRGLNSLESTLTFPASTSTIF

SEQ ID NO: 292 - protein, 245 aa, Homo sapiens
MKHTQSGQSTSPLVIDFTCRVCQMAFVFSSLIPLLLMTPVFCLGNTSECFQNFSQSHKCILMHSPPSAMAELPPSANTSVCSTLYFYGIAIFLGSFVLSLLTIMVLLIRAQTLYKKFVKSTGFLGSEQWAVIHIVDQRVRFYPVAFFCCWGPAVILMIIKLTKPQDTKLRMALYVLQALTATSQGLLNCGVYGWTQHKFHQLKQEARRDADTQTPLLCSQKRFYSRGLNSLESTLTFPASTSTIF

SEQ ID NO: 293 - protein, 376 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAGVLKKTTGLVGLAVCNTPHERLRILYTKILDVLEEIPKNAAYRKYTEQIINEKLAMVKAEPDVKKLEDQLQGGQLEEVILQAEHELNLARKMREWKLWEPLVEEPPADQWKWPI

SEQ ID NO: 294 - protein, 116 aa, Homo sapiens [sequence omitted]

SEQ ID NO: 295 - protein, 376 aa, synthetic construct; identical to SEQ ID NO: 293

SEQ ID NO: 296 - protein, 116 aa, Homo sapiens [sequence omitted]

SEQ ID NO: 297 - protein, 1261 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPTCGCHQKYALHSDGRTCIETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNGKDCVETGKCLSRAKTSPRAQLSCSKAGGVESCFLSCPAHTLFVPDSENSYVLSCGVPGPQGKALQKRNGTSSGLGPSCSDAPTTPIKQKARFKIRDAKCHLRPHSQARAKETARQPLLDHCHVTFVTLKCDSSKKRRRGRKSPSKEVSHITAEFEIETKMEEASDTCEADCLRKRAEQSLQAAIKTLRKSIGRQQFYVQVSGTEYEVAQRPAKALEGQGACGAGQVLQDSKCVACGPGTHFGGELGQCVSCMPGTYQDMEGQLSCTPCPSSDGLGLPGARNVSECGGQCSPGFFSADGFKPCQACPVGTYQPEPGRTGCFPCGGGLLTKHEGTTSFQDCEAKVHCSPGHHYNTTTHRCIRCPVGTYQPEFGQNHCITCPGNTSTDFDGSTNVTHCKNQHCGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDEDYQQLIEDIVRDGRLYASENHQEILKDKKLIKALFDVLAHPQNYFKYTAQESKEMFPRSFIKLLRSKVSRFLRPYKNPAFLYKVVKASS

SEQ ID NO: 298 - protein, 988 aa, Homo sapiens
MGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPTCGCHQKYALHSDGRTCIETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNGKDCVETGKCLSRAKTSPRAQLSCSKAGGVESCFLSCPAHTLFVPDSENSYVLSCGVPGPQGKALQKRNGTSSGLGPSCSDAPTTPIKQKARFKIRDAKCHLRPHSQARAKETARQPLLDHCHVTFVTLKCDSSKKRRRGRKSPSKEVSHITAEFEIETKMEEASDTCEADCLRKRAEQSLQAAIKTLRKSIGRQQFYVQVSGTEYEVAQRPAKALEGQGACGAGQVLQDSKCVACGPGTHFGGELGQCVSCMPGTYQDMEGQLSCTPCPSSDGLGLPGARNVSECGGQCSPGFFSADGFKPCQACPVGTYQPEPGRTGCFPCGGGLLTKHEGTTSFQDCEAKVHCSPGHHYNTTTHRCIRCPVGTYQPEFGQNHCITCPGNTSTDFDGSTNVTHCKNQHCGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDEDYQQLIEDIVRDGRLYASENHQEILKDKKLIKALFDVLAHPQNYFKYTAQESKEMFPRSFIKLLRSKVSRFLRPYK

SEQ ID NO: 299 - protein, 1103 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTVLQEPVQAAIWQALNHYAYRDAVFLAERLYAEVHSEEALFLLATCYYRSGKAYKAYRLLKGHSCTTPQCKYLLAKCCVDLSKLAEGEQILSGGVFNKQKSHDDIVTEFGDSACFTLSLLGHVYCKTDRLAKGSECYQKSLSLNPFLWSPFESLCEIGEKPDPDQTFKFTSLQNFSNCLPNSCTTQVPNHSLSHRQPETVLTETPQDTIELNRLNLESSNSKYSLNTDSSVSYIDSAVISPDTVPLGTGTSILSKQVQNKPKTGRSLLGGPAALSPLTPSFGILPLETPSPGDGSYLQNYTNTPPVIDVPSTGAPSKKTFRVLQSVARIGQTGTKSVFSQSGNSREVTPILAQTQSSGPQTSTTPQVLSPTITSPPNALPRRSSRLFTSDSSTTKENSKKLKMKFPPEIPNRKTKSKTNKGGITQPNINDSLEITKLDSSIISEGKISTITPQIQAFNLQKAAAEGLMSLLREMGKGYLALCSYNCKEAINILSHLPSHHYNTGWVLCQIGRAYFELSEYMQAERIFSEVRRIENYRVEGMEIYSTTLWHLQKDVALSVLSKDLTDMDKNSPEAWCAAGNCFSLQREHDIAIKFFQRAIQVDPNYAYAYTLLGHEFVLTEELDKALACFRNAIRVNPRHYNAWYGLGMIYYKQEKFSLAEMHFQKALDINPQSSVLLCHIGVVQHALKKSEKALDTLNKAIVIDPKNPLCKFHRASVLFANEKYKSALQELEELKQIVPKESLVYFLIGKVYKKLGQTHLALMNFSWAMDLDPKGANNQIKEAIDKRYLPDDEEPITQEEQIMGTDESQESSMTDADDTQLHAAESDEFNPAFLYKVVKASS

SEQ ID NO: 300 - protein, 830 aa, Homo sapiens
MTVLQEPVQAAIWQALNHYAYRDAVFLAERLYAEVHSEEALFLLATCYYRSGKAYKAYRLLKGHSCTTPQCKYLLAKCCVDLSKLAEGEQILSGGVFNKQKSHDDIVTEFGDSACFTLSLLGHVYCKTDRLAKGSECYQKSLSLNPFLWSPFESLCEIGEKPDPDQTFKFTSLQNFSNCLPNSCTTQVPNHSLSHRQPETVLTETPQDTIELNRLNLESSNSKYSLNTDSSVSYIDSAVISPDTVPLGTGTSILSKQVQNKPKTGRSLLGGPAALSPLTPSFGILPLETPSPGDGSYLQNYTNTPPVIDVPSTGAPSKKTFRVLQSVARIGQTGTKSVFSQSGNSREVTPILAQTQSSGPQTSTTPQVLSPTITSPPNALPRRSSRLFTSDSSTTKENSKKLKMKFPPEIPNRKTKSKTNKGGITQPNINDSLEITKLDSSIISEGKISTITPQIQAFNLQKAAAEGLMSLLREMGKGYLALCSYNCKEAINILSHLPSHHYNTGWVLCQIGRAYFELSEYMQAERIFSEVRRIENYRVEGMEIYSTTLWHLQKDVALSVLSKDLTDMDKNSPEAWCAAGNCFSLQREHDIAIKFFQRAIQVDPNYAYAYTLLGHEFVLTEELDKALACFRNAIRVNPRHYNAWYGLGMIYYKQEKFSLAEMHFQKALDINPQSSVLLCHIGVVQHALKKSEKALDTLNKAIVIDPKNPLCKFHRASVLFANEKYKSALQELEELKQIVPKESLVYFLIGKVYKKLGQTHLALMNFSWAMDLDPKGANNQIKEAIDKRYLPDDEEPITQEEQIMGTDESQESSMTDADDTQLHAAESDEF

SEQ ID NO: 301 - protein, 443 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKLLFPIFASLMLQYQVNTEFIGLRRCLMGLGRCRDHCNVDEKEIQKCKMKKCCVGPKVVKLIKNYLQYGTPNVLNEDVQEMLKPAKNSSAVIQRKHILSVLPQIKSTSFFANTNFVIIPNATPMNSATISTMTPGQITYTATSTKSNTKESRDSATASPPPAPPPPNILPTPSLELEEAEEQ

SEQ ID NO: 302 - protein, 183 aa, Homo sapiens
MKLLFPIFASLMLQYQVNTEFIGLRRCLMGLGRCRDHCNVDEKEIQKCKMKKCCVGPKVVKLIKNYLQYGTPNVLNEDVQEMLKPAKNSSAVIQRKHILSVLPQIKSTSFFANTNFVIIPNATPMNSATISTMTPGQITYTATSTKSNTKESRDSATASPPPAPPPPNILPTPSLELEEAEEQ

SEQ ID NO: 303 - protein, 611 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDQNNSLPPYAQGLASPQGAMTPGIPIFSPMMPYGTGLTPQPIQNTNSLSILEEQQRQQQQQQQQQQQQQQQQQQQQQQQQQQQQQQQQQQQQQAVAAAAVQQSTSQQATQGTSGQAPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTTNPAFLYKVVKASS

SEQ ID NO: 304 - protein, 338 aa, Homo sapiens [sequence omitted]

SEQ ID NO: 305 - protein, 950 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAEEKKLKLSNTVLPSESMKVVAESMGIAQIQEETCQLLTDEVSYRIKEIAQDALKFMHMGKRQKLTTSDIDYALKLKNVEPLYGFHAQEFIPFRFASGGGRELYFYEEKEVDLSDIINTPLPRVPLDVCLKAHWLSIEGCQPAIPENPPPAPKEQQKAEATEPLKSAKPGQEEDGPLKGKGQGATTADGKGKEKKAPPLLEGAPLRLKPRSIHELSVEQQLYYKEITEACVGSCEAKRAEALQSIATDPGLYQMLPRFSTFISEGVRVNVVQNNLALLIYLMRMVKALMDNPTLYLEKYVHELIPAVMTCIVSRQLCLRPDVDNHWALRDFAARLVAQICKHFSTTTNNIQSRITKTFTKSWVDEKTPWTTRYGSIAGLAELGHDVIKTLILPRLQQEGERIRSVLDGPVLSNIDRIGADHVQSLLLKHCAPVLAKLRPPPDNQDAYRAEFGSLGPLLCSQVVKARAQAALQAQQVNRTTLTITQPRPTLTLSQAPQPGPRTPGLLKVPGSIALPVQTLVSARAAAPPQPSPPPTKFIVMSSSSSAPSTQQVLSLSTSAPGSGSTTTSPVTTTVPSVQPIVKLVSTATTAPPSTAPSGPGSVQKYIVVSLPPTGEGKGGPTSHPSPVPPPASSPSPLSGSALCGGKQEAGDSPPPAPGTPKANGSQPNSGSPQPAPNPAFLYKVVKASS

SEQ ID NO: 306 - protein, 677 aa, Homo sapiens
MAEEKKLKLSNTVLPSESMKVVAESMGIAQIQEETCQLLTDEVSYRIKEIAQDALKFMHMGKRQKLTTSDIDYALKLKNVEPLYGFHAQEFIPFRFASGGGRELYFYEEKEVDLSDIINTPLPRVPLDVCLKAHWLSIEGCQPAIPENPPPAPKEQQKAEATEPLKSAKPGQEEDGPLKGKGQGATTADGKGKEKKAPPLLEGAPLRLKPRSIHELSVEQQLYYKEITEACVGSCEAKRAEALQSIATDPGLYQMLPRFSTFISEGVRVNVVQNNLALLIYLMRMVKALMDNPTLYLEKYVHELIPAVMTCIVSRQLCLRPDVDNHWALRDFAARLVAQICKHFSTTTNNIQSRITKTFTKSWVDEKTPWTTRYGSIAGLAELGHDVIKTLILPRLQQEGERIRSVLDGPVLSNIDRIGADHVQSLLLKHCAPVLAKLRPPPDNQDAYRAEFGSLGPLLCSQVVKARAQAALQAQQVNRTTLTITQPRPTLTLSQAPQPGPRTPGLLKVPGSIALPVQTLVSARAAAPPQPSPPPTKFIVMSSSSSAPSTQQVLSLSTSAPGSGSTTTSPVTTTVPSVQPIVKLVSTATTAPPSTAPSGPGSVQKYIVVSLPPTGEGKGGPTSHPSPVPPPASSPSPLSGSALCGGKQEAGDSPPPAPGTPKANGSQPNSGSPQPAP

SEQ ID NO: 307 - protein, 378 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEEKYGGDVLAGPGGGGGLGPVDVPSARLTKYIVLLCFTKFLKAVGLFESYDLLKAVHIVQFIFILKLGTAFFMVLFQKPFSSGKTITKHQIIGSLKIPGRKEFKDKKLNDPRKLVGN

SEQ ID NO: 308 - protein, 118 aa, Homo sapiens
MEEKYGGDVLAGPGGGGGLGPVDVPSARLTKYIVLLCFTKFLKAVGLFESYDLLKAVHIVQFIFILKLGTAFFMVLFQKPFSSGKTITKHQIIGSLKIPGRKEFKDKKLNDPRKLVGN

SEQ ID NO: 309 - protein, 651 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNEKSCSFHSKEELRDGQGERLSAGYSPSYDKDKSVLAFRGIPISELKNHGILQALTTEAYEWEPRVVSTEVVRAQEEWEAVDTIQPETGSQASSEQPGQLISFSEALQHFQTVDLSPFKKRIQPTIRRTGLAALRHYLFGPPKLHQRLREERDLVLTIAQCGLDSQDPVHGRVLQTIYKKLTGSKFDCALHGNHWEDLGFQGANPATDLRGAGFLALLHLLYLVMDSKTLPMAQEIFRLSRHHIQQFPFCLMSVNITHIAIQALREECLSRECNRQQKVIPVVNSFYAATFLHLAHVWRTQRKTISDSGFVLKGVLFLLGRPRLNAQCPRSREPKVVARLVLAAVLPHPHFLKFQLTKISITHPLESASSPFSALTVALFWSYTYDKHIF

SEQ ID NO: 310 - protein, 391 aa, Homo sapiens
MNEKSCSFHSKEELRDGQGERLSAGYSPSYDKDKSVLAFRGIPISELKNHGILQALTTEAYEWEPRVVSTEVVRAQEEWEAVDTIQPETGSQASSEQPGQLISFSEALQHFQTVDLSPFKKRIQPTIRRTGLAALRHYLFGPPKLHQRLREERDLVLTIAQCGLDSQDPVHGRVLQTIYKKLTGSKFDCALHGNHWEDLGFQGANPATDLRGAGFLALLHLLYLVMDSKTLPMAQEIFRLSRHHIQQFPFCLMSVNITHIAIQALREECLSRECNRQQKVIPVVNSFYAATFLHLAHVWRTQRKTISDSGFVLKGVLFLLGRPRLNAQCPRSREPKVVARLVLAAVLPHPHFLKFQLTKISITHPLESASSPFSALTVALFWSYTYDKHIF

SEQ ID NO: 311 - protein, 685 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEQGLEEEEEVDPRIQGELEKLNQSTDDINRRETELEDARQKFRSVLVEATVKLDELVKKIGKAVEDSKPYWEARRVARQAQLEAQKATQDFQRATEVLRAAKETISLAEQRLLEDDKRQFDSAWQEMLNHATQRVMEAEQTKTRSELVHKETAARYNAAMGRMRQLEKKLKRAINKSKPYFELKAKYYVQLEQLKKTVDDLQAKLTLAKGEYKMALKNLEMISDEIHERRRSSAMGPRGCGVGAEGSSTSVEDLPGSKPEPDAISVASEAFEDDSCSNFVSEDDSETQSVSSFSSGPTSPSEMPDQFPAVVRPGSLDLPSPVSLSEFGMMFPVLGPRSECSGASSPECEVERGDRAEGAENKTSDKANNNRGLSSSSGSGGSSKSQSSTSPEGQALENRMKQLSLQCSKGRDGIIADIKMVQIG

SEQ ID NO: 312 - protein, 425 aa, Homo sapiens
MEQGLEEEEEVDPRIQGELEKLNQSTDDINRRETELEDARQKFRSVLVEATVKLDELVKKIGKAVEDSKPYWEARRVARQAQLEAQKATQDFQRATEVLRAAKETISLAEQRLLEDDKRQFDSAWQEMLNHATQRVMEAEQTKTRSELVHKETAARYNAAMGRMRQLEKKLKRAINKSKPYFELKAKYYVQLEQLKKTVDDLQAKLTLAKGEYKMALKNLEMISDEIHERRRSSAMGPRGCGVGAEGSSTSVEDLPGSKPEPDAISVASEAFEDDSCSNFVSEDDSETQSVSSFSSGPTSPSEMPDQFPAVVRPGSLDLPSPVSLSEFGMMFPVLGPRSECSGASSPECEVERGDRAEGAENKTSDKANNNRGLSSSSGSGGSSKSQSSTSPEGQALENRMKQLSLQCSKGRDGIIADIKMVQIG

SEQ ID NO: 313 - protein, 525 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNRFLLLMSLYLLGSARGTSSQPNELSGSIDHQTSVQQLPGEFFSLENPSDAEALYETSSGLNTLSEHGSSEHGSSKHTVAEHTSGEHAESEHASGEPAATEHAEGEHTVGEQPSGEQPSGEHLSGEQPLSELESGEQPSDEQPSGEHGSGEQPSGEQASGEQPSGEHASGEQASGAPISSTSTGTILNCYTCAYMNDQGKCLRGEGTCITQNSQQCMLKKIFEGGKLQFMVQGCENMCPSMNLFSHGTRMQIICCRNQSFCNKI

SEQ ID NO: 314 - protein, 265 aa, Homo sapiens
MNRFLLLMSLYLLGSARGTSSQPNELSGSIDHQTSVQQLPGEFFSLENPSDAEALYETSSGLNTLSEHGSSEHGSSKHTVAEHTSGEHAESEHASGEPAATEHAEGEHTVGEQPSGEQPSGEHLSGEQPLSELESGEQPSDEQPSGEHGSGEQPSGEQASGEQPSGEHASGEQASGAPISSTSTGTILNCYTCAYMNDQGKCLRGEGTCITQNSQQCMLKKIFEGGKLQFMVQGCENMCPSMNLFSHGTRMQIICCRNQSFCNKI

SEQ ID NO: 315 - protein, 498 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMCRRCKPELGQNFQSASGICICHWLQIRRTRSREGRAMWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHYMLLVFVKVPILLILVTAILWLKGSQRVPEEPGEQPIYMNFSEPLTKDMAT

SEQ ID NO: 316 - protein, 238 aa, Homo sapiens
MCRRCKPELGQNFQSASGICICHWLQIRRTRSREGRAMWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHYMLLVFVKVPILLILVTAILWLKGSQRVPEEPGEQPIYMNFSEPLTKDMAT

SEQ ID NO: 317 - protein, 521 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSRRYDSRTTIFSPEGRLYQVEYAMEAIGHAGTCLGILANDGVLLAAERRNIHKLLDEVFFSEKIYKLNEDMACSVAGITSDANVLTNELRLIAQRYLLQYQEPIPCEQLVTALCDIKQAYTQFGGKRPFGVSLLYIGWDKHYGFQLYQSDPSGNYGGWKATCIGNNSAAAVSMLKQDYKEGEMTLKSALALAIKVLNKTMDVSKLSAEKVEIATLTRENGKTVIRVLKQKEVEQLIKKHEEEEAKAEREKKEKEQKEKDK

SEQ ID NO: 318 - protein, 261 aa, Homo sapiens
MSRRYDSRTTIFSPEGRLYQVEYAMEAIGHAGTCLGILANDGVLLAAERRNIHKLLDEVFFSEKIYKLNEDMACSVAGITSDANVLTNELRLIAQRYLLQYQEPIPCEQLVTALCDIKQAYTQFGGKRPFGVSLLYIGWDKHYGFQLYQSDPSGNYGGWKATCIGNNSAAAVSMLKQDYKEGEMTLKSALALAIKVLNKTMDVSKLSAEKVEIATLTRENGKTVIRVLKQKEVEQLIKKHEEEEAKAEREKKEKEQKEKDK

SEQ ID NO: 319 - protein, 650 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVVMPLAQIKQPMTLGTITKRTGKVKPPPQISPSKSMGGEFCVAAIFGTSRSWFANNAGLKREKDQSKQVVVESLYIISCYGTLVEHMMEPRPLSTAPKISDDTPLEMMTSPRASWTLVRTPQWNELQPPFNANHPLLLAADAVQYYQFLLAGLVPPGSPGPITRHGSYDSLASDHSGQEDEEWLSQVEIVTHTGPHRRLWMGPQFQFKTIHPSGQTTVISSSSSVLQSHGPSDTPQPLLDFDTDDLDLNSLRIQPVRSDPVSMPGSSRPVSDRRGVSTVIDAASGTFDRSVTLLEVCGSWPEGFGLRHMSSMEHTEEGLRERLADAMAESPSRDVVGSGTELQREGSIETLSNSSGSTSGSIPRNFDGYRSPLPTNESQPLSLFPTGFP

SEQ ID NO: 320 - protein, 390 aa, Homo sapiens
MVVMPLAQIKQPMTLGTITKRTGKVKPPPQISPSKSMGGEFCVAAIFGTSRSWFANNAGLKREKDQSKQVVVESLYIISCYGTLVEHMMEPRPLSTAPKISDDTPLEMMTSPRASWTLVRTPQWNELQPPFNANHPLLLAADAVQYYQFLLAGLVPPGSPGPITRHGSYDSLASDHSGQEDEEWLSQVEIVTHTGPHRRLWMGPQFQFKTIHPSGQTTVISSSSSVLQSHGPSDTPQPLLDFDTDDLDLNSLRIQPVRSDPVSMPGSSRPVSDRRGVSTVIDAASGTFDRSVTLLEVCGSWPEGFGLRHMSSMEHTEEGLRERLADAMAESPSRDVVGSGTELQREGSIETLSNSSGSTSGSIPRNFDGYRSPLPTNESQPLSLFPTGFP

SEQ ID NO: 321 - protein, 546 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMERRLGVRAWVKENRGSFQPPVCNKLMHQEQLKVMFIGGPNTRKDYHIEEGEEVFYQLEGDMVLRVLEQGKHRDVVIRQGEIFLLPARVPHSPQRFANTVGLVVERRRLETELDGLRYYVGDTMDVLFEKWFYCKDLGTQLAPIIQEFFSSEQYRTGKPIPDQLLKEPPFPLSTRSIMEPMSLDAWLDSHHRELQAGTPLSLFGDTYETQVIAYGQGSSEGLRQNVDVWLWQLEGSSVVTMGGRRLSLAPDDSLLVLAGTSYAWERTQGSVALSVTQDPACKKPLG

SEQ ID NO: 322 - protein, 286 aa, Homo sapiens
MERRLGVRAWVKENRGSFQPPVCNKLMHQEQLKVMFIGGPNTRKDYHIEEGEEVFYQLEGDMVLRVLEQGKHRDVVIRQGEIFLLPARVPHSPQRFANTVGLVVERRRLETELDGLRYYVGDTMDVLFEKWFYCKDLGTQLAPIIQEFFSSEQYRTGKPIPDQLLKEPPFPLSTRSIMEPMSLDAWLDSHHRELQAGTPLSLFGDTYETQVIAYGQGSSEGLRQNVDVWLWQLEGSSVVTMGGRRLSLAPDDSLLVLAGTSYAWERTQGSVALSVTQDPACKKPLG

SEQ ID NO: 323 - protein, 425 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMADFDEIYEEEEDEERALEEQLLKYSPDPVVVRGSGHVTVFGLSNKFESEFPSSLTGKVAPEEFKASINRVNSCLKKNLPVNVRWLLCGCLCCCCTLGCSMWPVICLSKRTRRSIEKLLEWENNRLYHKLCLHWRLSKRKCETNNMMEYVILIEFLPKTPIFRPD

SEQ ID NO: 324 - protein, 165 aa, Homo sapiens
MADFDEIYEEEEDEERALEEQLLKYSPDPVVVRGSGHVTVFGLSNKFESEFPSSLTGKVAPEEFKASINRVNSCLKKNLPVNVRWLLCGCLCCCCTLGCSMWPVICLSKRTRRSIEKLLEWENNRLYHKLCLHWRLSKRKCETNNMMEYVILIEFLPKTPIFRPD

SEQ ID NO: 325 - protein, 425 aa, synthetic construct; identical to SEQ ID NO: 323

SEQ ID NO: 326 - protein, 165 aa, Homo sapiens; identical to SEQ ID NO: 324

SEQ ID NO: 327 - protein, 426 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMRLTEKSEGEQQLKPNNSNAPNEDQEEEIQQSEQHTPARQRTQRADTQPSRCRLPSRRTPTTSSDRTINLLEVLPWPTEWIFNPYRLPALFELYPEFLLVFKEAFHDISHCLKAQMEKIGLPIILHLFALSTLYFYKFFLPTILSLSFFILLVLLLFIIVFILIFF

SEQ ID NO: 328 - protein, 166 aa, Homo sapiens
MRLTEKSEGEQQLKPNNSNAPNEDQEEEIQQSEQHTPARQRTQRADTQPSRCRLPSRRTPTTSSDRTINLLEVLPWPTEWIFNPYRLPALFELYPEFLLVFKEAFHDISHCLKAQMEKIGLPIILHLFALSTLYFYKFFLPTILSLSFFILLVLLLFIIVFILIFF

SEQ ID NO: 329 - protein, 697 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAGTLYTYPENWRAFKALIAAQYSGAQVRVLSAPPHFHFGQTNRTPEFLRKFPAGKVPAFEGDDGFCVFESNAIAYYVSNEELRGSTPEAAAQVVQWVSFADSDIVPPASTWVFPTLGIMHHNKQATENAKEEVRRILGLLDAYLKTRTFLVGERVTLADITVVCTLLWLYKQVLEPSFRQAFPNTNRWFLTCINQPQFRAVLGEVKLCEKMAQFDAKKFAETQPKKDTPRKEKGSREEKQKPQAERKEEKKAAAPAPEEEMDECEQALAAEPKAKDPFAHLPKSTFVLDEFKRKYSNEDTLSVALPYFWEHFDKDGWSLWYSEYRFPEELTQTFMSCNLITGMFQRLDKLRKNAFASVILFGTNNSSSISGVWVFRGQELAFPLSPDWQVDYESYTWRKLDPGSEETQTLVREYFSWEGAFQHVGKAFNQGKIFK

SEQ ID NO: 330 - protein, 437 aa, Homo sapiens
MAAGTLYTYPENWRAFKALIAAQYSGAQVRVLSAPPHFHFGQTNRTPEFLRKFPAGKVPAFEGDDGFCVFESNAIAYYVSNEELRGSTPEAAAQVVQWVSFADSDIVPPASTWVFPTLGIMHHNKQATENAKEEVRRILGLLDAYLKTRTFLVGERVTLADITVVCTLLWLYKQVLEPSFRQAFPNTNRWFLTCINQPQFRAVLGEVKLCEKMAQFDAKKFAETQPKKDTPRKEKGSREEKQKPQAERKEEKKAAAPAPEEEMDECEQALAAEPKAKDPFAHLPKSTFVLDEFKRKYSNEDTLSVALPYFWEHFDKDGWSLWYSEYRFPEELTQTFMSCNLITGMFQRLDKLRKNAFASVILFGTNNSSSISGVWVFRGQELAFPLSPDWQVDYESYTWRKLDPGSEETQTLVREYFSWEGAFQHVGKAFNQGKIFK

SEQ ID NO: 331 - protein, 959 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNLLPKSSREFGSVDYWEKFFQQRGKKAFEWYGTYLELCGVLHKYIKPREKVLVIGCGNSELSEQLYDVGYRDIVNIDISEVVIKQMKECNATRRPQMSFLKMDMTQMEFPDASFQVVLDKGTLDAVLTDEEEKTLQQVDRMLAEVGRVLQVGGRYLCISLAQAHILKKAVGHFSREGWMVRVHQVANSQDQVLEAEPQFSLPVFAFIMTKFRPVPGSALQIFELCAQEQRKPVRLESAERLAEAVQERQQYAWLCSQLRRKARLGSVSLDLCDGDTGEPRYTLHVVDSPTVKPSRDNHFAIFIIPQGRETEWLFGMDEGRKQLAASAGFRRLITVALHRGQQYESMDHIQAELSARVMELAPAGMPTQQQVPFLSVGGDIGVRTVQHQDCSPLSGDYVIEDVQGDDKRYFRRLIFLSNRNVVQSEARLLKDVSHKAQKKRKKDRKKQRPADAEDLPAAPGQSIDKSYLCCEHHKAMIAGLALLRNPELLLEIPLALLVVGLGGGSLPLFVHDHFPKSCIDAVEIDPSMLEVATQWFGFSQSDRMKVHIADGLDYIASLAGGGEARPCYDVIMFDVDSKDPTLGMSCPPPAFVEQSFLQKVKSILTPEGVFILNLVCRDLGLKDSVLAGLKAVFPLLYVRRIEGEVNEILFCQLHPEQKLATPELLETAQALERTLRKPGRGWDDTYVLSDMLKTVKIV

SEQ ID NO: 332 - protein, 699 aa, Homo sapiens
MNLLPKSSREFGSVDYWEKFFQQRGKKAFEWYGTYLELCGVLHKYIKPREKVLVIGCGNSELSEQLYDVGYRDIVNIDISEVVIKQMKECNATRRPQMSFLKMDMTQMEFPDASFQVVLDKGTLDAVLTDEEEKTLQQVDRMLAEVGRVLQVGGRYLCISLAQAHILKKAVGHFSREGWMVRVHQVANSQDQVLEAEPQFSLPVFAFIMTKFRPVPGSALQIFELCAQEQRKPVRLESAERLAEAVQERQQYAWLCSQLRRKARLGSVSLDLCDGDTGEPRYTLHVVDSPTVKPSRDNHFAIFIIPQGRETEWLFGMDEGRKQLAASAGFRRLITVALHRGQQYESMDHIQAELSARVMELAPAGMPTQQQVPFLSVGGDIGVRTVQHQDCSPLSGDYVIEDVQGDDKRYFRRLIFLSNRNVVQSEARLLKDVSHKAQKKRKKDRKKQRPADAEDLPAAPGQSIDKSYLCCEHHKAMIAGLALLRNPELLLEIPLALLVVGLGGGSLPLFVHDHFPKSCIDAVEIDPSMLEVATQWFGFSQSDRMKVHIADGLDYIASLAGGGEARPCYDVIMFDVDSKDPTLGMSCPPPAFVEQSFLQKVKSILTPEGVFILNLVCRDLGLKDSVLAGLKAVFPLLYVRRIEGEVNEILFCQLHPEQKLATPELLETAQALERTLRKPGRGWDDTYVLSDMLKTVKIV

SEQ ID NO: 333 - protein, 496 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMILIGRMSAQVPMNMTITGCMMTFYRTTPAVLFWQWINQSFNAVVNYTNRSGDAPLTVNELGTAYVSATTGAVATALGLNALTKHVSPLIGRFVPFAAVAAANCINIPLMRQRELKVGIPVTDENGNRLGESANAAKQAITQVVVSRILMAAPGMAIPPFIMNTLEKKAFLKRFPWMSAPIQVGLVGFCLVFATPLCCALFPQKSSMSVTSLEAELQAKIQESHPELRRVYFNKGL

SEQ ID NO: 334 - protein, 236 aa, Homo sapiens
MILIGRMSAQVPMNMTITGCMMTFYRTTPAVLFWQWINQSFNAVVNYTNRSGDAPLTVNELGTAYVSATTGAVATALGLNALTKHVSPLIGRFVPFAAVAAANCINIPLMRQRELKVGIPVTDENGNRLGESANAAKQAITQVVVSRILMAAPGMAIPPFIMNTLEKKAFLKRFPWMSAPIQVGLVGFCLVFATPLCCALFPQKSSMSVTSLEAELQAKIQESHPELRRVYFNKGL

SEQ ID NO: 335 - protein, 633 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTGWSCLVTGAGGFLGQRIIRLLVKEKELKEIRVLDKAFGPELREEFSKLQNKTKLTVLEGDILDEPFLKRACQDVSVIIHTACIIDVFGVTHRESIMNVNVKGTQLLLEACVQASVPVFIYTSSIEVAGPNSYKEIIQNGHEEEPLENTWPAPYPHSKKLAEKAVLAANGWNLKNGGTLYTCALRPMYIYGEGSRFLSASINEALNNNGILSSVGKFSTVNPVYVGNVAWAHILALRALQDPKKAPSIRGQFYYISDDTPHQSYDNLNYTLSKEFGLRLDSRWSFPLSLMYWIGFLLEIVSFLLRPIYTYRPPFNRHIVTLSNSVFTFSYKKAQRDLAYKPLYSWEEAKQKTVEWVGSLVDRHKENLKSKTQ

SEQ ID NO: 336 - protein, 373 aa, Homo sapiens [sequence omitted]

SEQ ID NO: 337 - protein, 534 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNISIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 338 - protein, 274 aa, Homo sapiens
MGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNISIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 339 - protein, 553 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMIDHLSRAVISDPEQNLAIEQKESDHILPDSKMTPLRFRKRTLHETKIRTHSTLTENVLSHKLQFDGRIVSRTNVLPFIQKSIYSHQCGRRKGKQYRLGDFYVGATLTFLSSDHLSLPESIKENTLLKLRITNIDQIALDSLKTASMEQEDDIIIQETNDRLVFKAIQDVLKEKLHKRGVRILTGLGKYFQQLDKEGNGLLDKADFKQALKVFHLEVSEKDFESAWLILNDNGNGKVDYGEFKRGIIGEMNEYRKSYVRKAFMKLDFNKSGSVPIINIRKCYCAKKHSQVISG

SEQ ID NO: 340 - protein, 293 aa, Homo sapiens
MIDHLSRAVISDPEQNLAIEQKESDHILPDSKMTPLRFRKRTLHETKIRTHSTLTENVLSHKLQFDGRIVSRTNVLPFIQKSIYSHQCGRRKGKQYRLGDFYVGATLTFLSSDHLSLPESIKENTLLKLRITNIDQIALDSLKTASMEQEDDIIIQETNDRLVFKAIQDVLKEKLHKRGVRILTGLGKYFQQLDKEGNGLLDKADFKQALKVFHLEVSEKDFESAWLILNDNGNGKVDYGEFKRGIIGEMNEYRKSYVRKAFMKLDFNKSGSVPIINIRKCYCAKKHSQVISG

SEQ ID NO: 341 - protein, 665 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLSSFLSPQNGTWADTFSLLLALAVALYLGYYWACVLQRPRLVAGPQFLAFLEPHCSITTETFYPTLWCFEGRLQSIFQVLLQSQPLVLYQSDILQTPDGGQLLLDWAKQPDSSQDPDPTTQPIVLLLPGITGSSQETYVLHLVNQALRDGYQAVVFNNRGCRGEELRTHRAFCASNTEDLETVVNHIKHRYPQAPLLAVGISFGGILVLNHLAQARQAAGLVAALTLSACWDSFETTRSLETPLNSLLFNQPLTAGLCQLVERNRKVIEKVVDIDFVLQARTIRQFDERYTSVAFGYQDCVTYYKAASPRTKIDAIRIPVLYLSAADDPFSPVCALPIQAAQHSPYVALLITARGGHIGFLEGLLPWQHWYMSRLLHQYAKAIFQDPEGLPDLRALLPSEDRNS

SEQ ID NO: 342 - protein, 405 aa, Homo sapiens
MLSSFLSPQNGTWADTFSLLLALAVALYLGYYWACVLQRPRLVAGPQFLAFLEPHCSITTETFYPTLWCFEGRLQSIFQVLLQSQPLVLYQSDILQTPDGGQLLLDWAKQPDSSQDPDPTTQPIVLLLPGITGSSQETYVLHLVNQALRDGYQAVVFNNRGCRGEELRTHRAFCASNTEDLETVVNHIKHRYPQAPLLAVGISFGGILVLNHLAQARQAAGLVAALTLSACWDSFETTRSLETPLNSLLFNQPLTAGLCQLVERNRKVIEKVVDIDFVLQARTIRQFDERYTSVAFGYQDCVTYYKAASPRTKIDAIRIPVLYLSAADDPFSPVCALPIQAAQHSPYVALLITARGGHIGFLEGLLPWQHWYMSRLLHQYAKAIFQDPEGLPDLRALLPSEDRNS

SEQ ID NO: 343 - protein, 917 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDDPDCDSTWEEDEEDAEDAEDEDCEDGEAAGARDADAGDEDEESEEPRAARPSSFQSRMTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYELMLGFYGIRLEDRGTGTVGRAQNYQKRFQNLNWRSHNNLRITRILKSLGELGLEHFQAPLVRFFLEETLVRRELPGVRQSALDYFMFAVRCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKRRKVDEGAGDSAAVASGGAQTLALAGSPAPSGHPKAGHSENGVEEDTEGRTGPKEGTPGSPSETPGPSPAGPAGDEPAESPSETPGPRPAGPAGDEPAESPSETPGPSPAGPTRDEPAESPSETPGPRPAGPAGDEPAESPSETPGPRPAGPAGDEPAESPSETPGPSPAGPTRDEPAKAGEAAELQDAEVESSAKSGKP

SEQ ID NO: 344 - protein, 657 aa, Homo sapiens
MDDPDCDSTWEEDEEDAEDAEDEDCEDGEAAGARDADAGDEDEESEEPRAARPSSFQSRMTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYELMLGFYGIRLEDRGTGTVGRAQNYQKRFQNLNWRSHNNLRITRILKSLGELGLEHFQAPLVRFFLEETLVRRELPGVRQSALDYFMFAVRCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKRRKVDEGAGDSAAVASGGAQTLALAGSPAPSGHPKAGHSENGVEEDTEGRTGPKEGTPGSPSETPGPSPAGPAGDEPAESPSETPGPRPAGPAGDEPAESPSETPGPSPAGPTRDEPAESPSETPGPRPAGPAGDEPAESPSETPGPRPAGPAGDEPAESPSETPGPSPAGPTRDEPAKAGEAAELQDAEVESSAKSGKP

SEQ ID NO: 345 - protein, 1039 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEEKDYSEADGLSERTTPSKAQKSPQKIAKKYKSAICRVTLLDASEYECEVEKHGRGQVLFDLVCEHLNLLEKDYFGLTFCDADSQKNWLDPSKEIKKQIRSSPWNFAFTVKFYPPDPAQLTEDITRYYLCLQLRADIITGRLPCSFVTHALLGSYAVQAELGDYDAEEHVGNYVSELRFAPNQTRELEERIMELHKTYRGMTPGEAEIHFLENAKKLSMYGVDLHHAKDSEGIDIMLGVCANGLLIYRDRLRINRFAWPKILKISYKRSNFYIKIRPGEYEQFESTIGFKLPNHRSAKRLWKVCIEHHTFFRLVSPEPPPKGFLVMGSKFRYSGRTQAQTRQASALIDRPAPFFERSSSKRYTMSRSLDGAEFSRPASVSENHDAGPDGDKRDEDGESGGQRSEAEEGEVRTPTKIKELKFLDKPEDVLLKHQASINELKRTLKEPNSKLIHRDRDWERERRLPSSPASPSPKGTPEKANERAGLREGSEEKVKPPRPRAPESDTGDEDQDQERDTVFLKDNHLAIERKCSSITVSSTSSLEAEVDFTVIGDYHGSAFEDFSRSLPELDRDKSDSDTEGLLFSRDLNKGAPSQDDESGGIEDSPDRGACSTPDMPQFEPVKTETMTVSSLAIRKKIEPEAVLQTRVSAMDNTQENSLKSGKGAAAMIPGPQTVATEIRSLSPIIGKDVLTSTYGATAETLSTSTTTHVTKTVKGGFSETRIEKRIIITGDEDVDQDQALALAIKEAKLQHPDMLVTKAVVYRETDPSPEERDKKPQES

SEQ ID NO: 346 - protein, 779 aa, Homo sapiens
MEEKDYSEADGLSERTTPSKAQKSPQKIAKKYKSAICRVTLLDASEYECEVEKHGRGQVLFDLVCEHLNLLEKDYFGLTFCDADSQKNWLDPSKEIKKQIRSSPWNFAFTVKFYPPDPAQLTEDITRYYLCLQLRADIITGRLPCSFVTHALLGSYAVQAELGDYDAEEHVGNYVSELRFAPNQTRELEERIMELHKTYRGMTPGEAEIHFLENAKKLSMYGVDLHHAKDSEGIDIMLGVCANGLLIYRDRLRINRFAWPKILKISYKRSNFYIKIRPGEYEQFESTIGFKLPNHRSAKRLWKVCIEHHTFFRLVSPEPPPKGFLVMGSKFRYSGRTQAQTRQASALIDRPAPFFERSSSKRYTMSRSLDGAEFSRPASVSENHDAGPDGDKRDEDGESGGQRSEAEEGEVRTPTKIKELKFLDKPEDVLLKHQASINELKRTLKEPNSKLIHRDRDWERERRLPSSPASPSPKGTPEKANERAGLREGSEEKVKPPRPRAPESDTGDEDQDQERDTVFLKDNHLAIERKCSSITVSSTSSLEAEVDFTVIGDYHGSAFEDFSRSLPELDRDKSDSDTEGLLFSRDLNKGAPSQDDESGGIEDSPDRGACSTPDMPQFEPVKTETMTVSSLAIRKKIEPEAVLQTRVSAMDNTQENSLKSGKGAAAMIPGPQTVATEIRSLSPIIGKDVLTSTYGATAETLSTSTTTHVTKTVKGGFSETRIEKRIIITGDEDVDQDQALALAIKEAKLQHPDMLVTKAVVYRETDPSPEERDKKPQES

SEQ ID NO: 347 - protein, 664 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPYNFCLPSLSCRTSCSSRPCVPPSCHGYTLPGACNIPANVSNCNWFCEGSFNGSEKETMQFLNDRLASYLEKVRQLERDNAELENLIRERSQQQEPLLCPSYQSYFKTIEELQQKILCSKSENARLVVQIDNAKLAADDFRTKYQTEQSLRQLVESDINSLRRILDELTLCRSDLEAQMESLKEELLSLKQNHEQEVNTLRCQLGDRLNVEVDAAPAVDLNQVLNETRNQYEALVETNRREVEQWFATQTEELNKQVVSSSEQLQSYQAEIIELRRTVNALEIELQAQHNLRYSLENTLTESEARYSSQLSQVQSLITNVESQLAEIRSDLERQNQEYQVLLDVRARLECEINTYRSLLESEDCKLPSNPCATTNACEKPIGSCVTNPCGPRSRCGPCNTFGY

SEQ ID NO: 348 - protein, 404 aa, Homo sapiens
MPYNFCLPSLSCRTSCSSRPCVPPSCHGYTLPGACNIPANVSNCNWFCEGSFNGSEKETMQFLNDRLASYLEKVRQLERDNAELENLIRERSQQQEPLLCPSYQSYFKTIEELQQKILCSKSENARLVVQIDNAKLAADDFRTKYQTEQSLRQLVESDINSLRRILDELTLCRSDLEAQMESLKEELLSLKQNHEQEVNTLRCQLGDRLNVEVDAAPAVDLNQVLNETRNQYEALVETNRREVEQWFATQTEELNKQVVSSSEQLQSYQAEIIELRRTVNALEIELQAQHNLRYSLENTLTESEARYSSQLSQVQSLITNVESQLAEIRSDLERQNQEYQVLLDVRARLECEINTYRSLLESEDCKLPSNPCATTNACEKPIGSCVTNPCGPRSRCGPCNTFGY

SEQ ID NO: 349 - protein, 644 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMETSSPRPPRPSSNPGLSLDARLGVDTRLWAKVLFTALYALIWALGAAGNALSVHVVLKARAGRAGRLRHHVLSLALAGLLLLLVGVPVELYSFVWFHYPWVFGDLGCLAVCQPLCARSLLTPRRTRWLVALSWAASLGLALPMAVIMGQKHELETADGEPEPASRVCTVLVSRTALQVFIQVNVLVSFVLPLALTAFLNGVTVSHLLALCSQVPSTSTPGSSTPSRLELLSEEGLLSFIVWKKTFIQGGQVSLVRHKDVRRIRSLQRSVQVLRAIVVMYVICWLPYHARRLMYCYVPDDAWTDPLYNFYHYFYMVTNTLFYVSSAVTPLLYNAVSSSFRKLFLEAVSSLCGEHHPMKRLPPKPQSPTLMDTASGFGDPPETRT

SEQ ID NO: 350 - protein, 384 aa, Homo sapiens [sequence omitted]

SEQ ID NO: 351 - protein, 810 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRKGKQTRQQQEHQKYMVPLQASAQEKNGL

SEQ ID NO: 352 - protein, 550 aa, Homo sapiens
MAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRKGKQTRQQQEHQKYMVPLQASAQEKNGL

SEQ ID NO: 353 - protein, 710 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 354 - protein, 450 aa, Homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 355 - protein, 345 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKITGGLLLLCTVVYFCSSSEAASLSPKKVDCSIYKKYPVVAIPCPITYLPVCGSDYITYGNECHLCTESLKSNGRVQFLHDGSC

SEQ ID NO: 356 - protein, 85 aa, Homo sapiens
MKITGGLLLLCTVVYFCSSSEAASLSPKKVDCSIYKKYPVVAIPCPITYLPVCGSDYITYGNECHLCTESLKSNGRVQFLHDGSC

SEQ ID NO: 357 - protein, 1083 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKRHRPVSSSDSSDESPSTSFTSGSMYRIKSKIPNEHKKPAEVFRKDLISAMKLPDSHHINPDSYYLFADTWKEEWEKGVQVPASPDTVPQPSLRIIAEKVKDVLFIRPRKYIHCSSPDTTEPGYINIMELAASVCRYDLDDMDIFWLQELNEDLAEMGCGPVDENLMEKTVEVLERHCHENMNHAIETEEGLGIEYDEDVICDVCRSPDSEEGNDMVFCDKCNVCVHQACYGILKVPEGSWLCRSCVLGIYPQCVLCPKKGGALKTTKTGTKWAHVSCALWIPEVSIACPERMEPITKISHIPPSRWALVCNLCKLKTGACIQCSIKSCITAFHVTCAFEHGLEMKTILDEGDEVKFKSYCLKHSQNRQKLGEAEYPHHRAKEQSQAKSEKTSLRAQKLRELEEEFYSLVRVEDVAAELGMPTLAVDFIYNYWKLKRKSNFNKPLFPPKEDEENGLVQPKEESIHTRMRMFMHLRQDLERVRNLCYMISRREKLKLSHNKIQEQIFGLQVQLLNQEIDAGLPLTNALENSLFYPPPRITLKLKMPKSTPEDHRNSSTETDQQPHSPDSSSSVHSIRNMQVPQESLEMRTKSYPRYPLESKNNRLLASLSHSRSEAKESSPAWRTPSSECYHGQSLGKPLVLQAALHGQSSIGNGKSQPNSKFAKSNGLEGSWSGNVTQKDSSSEMFCDQEPVFSPHLVSQGSFRKSTVEHFSRSFKETTNRWVKNTEDLQCYVKPTKNMSPKEQFWGRQVLRRSAGRAPYQENDGYCPDLELSDSEAESDGNKEKVRVRKDSSDRENPPHDSRRDCHGKSKTHPLSHSSMQR

SEQ ID NO: 358 - protein, 823 aa, Homo sapiens
MKRHRPVSSSDSSDESPSTSFTSGSMYRIKSKIPNEHKKPAEVFRKDLISAMKLPDSHHINPDSYYLFADTWKEEWEKGVQVPASPDTVPQPSLRIIAEKVKDVLFIRPRKYIHCSSPDTTEPGYINIMELAASVCRYDLDDMDIFWLQELNEDLAEMGCGPVDENLMEKTVEVLERHCHENMNHAIETEEGLGIEYDEDVICDVCRSPDSEEGNDMVFCDKCNVCVHQACYGILKVPEGSWLCRSCVLGIYPQCVLCPKKGGALKTTKTGTKWAHVSCALWIPEVSIACPERMEPITKISHIPPSRWALVCNLCKLKTGACIQCSIKSCITAFHVTCAFEHGLEMKTILDEGDEVKFKSYCLKHSQNRQKLGEAEYPHHRAKEQSQAKSEKTSLRAQKLRELEEEFYSLVRVEDVAAELGMPTLAVDFIYNYWKLKRKSNFNKPLFPPKEDEENGLVQPKEESIHTRMRMFMHLRQDLERVRNLCYMISRREKLKLSHNKIQEQIFGLQVQLLNQEIDAGLPLTNALENSLFYPPPRITLKLKMPKSTPEDHRNSSTETDQQPHSPDSSSSVHSIRNMQVPQESLEMRTKSYPRYPLESKNNRLLASLSHSRSEAKESSPAWRTPSSECYHGQSLGKPLVLQAALHGQSSIGNGKSQPNSKFAKSNGLEGSWSGNVTQKDSSSEMFCDQEPVFSPHLVSQGSFRKSTVEHFSRSFKETTNRWVKNTEDLQCYVKPTKNMSPKEQFWGRQVLRRSAGRAPYQENDGYCPDLELSDSEAESDGNKEKVRVRKDSSDRENPPHDSRRDCHGKSKTHPLSHSSMQR

SEQ ID NO: 359 - protein, 469 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAHLLPICALFLTLLDMAQGFRGPLLPNRPFTTVWNANTQWCLERHGVDVDVSVFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCSG

SEQ ID NO: 360 - protein, 209 aa, Homo sapiens
MAAHLLPICALFLTLLDMAQGFRGPLLPNRPFTTVWNANTQWCLERHGVDVDVSVFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCSG

SEQ ID NO: 361 - protein, 468 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMATSAVPSDNLPTYKLVVVGDGGVGKSALTIQFFQKIFVPDYDPTIEDSYLKHTEIDNQWAILDVLDTAGQEEFSAMREQYMRTGDGFLIVYSVTDKASFEHVDRFHQLILRVKDRESFPMILVANKVDLMHLRKITREQGKEMATKHNIPYIETSAKDPPLNVDKAFHDLVRVIRQQIPEKSQKKKKKTKWRGDRATGTHKLQCVIL

SEQ ID NO: 362 - protein, 208 aa, Homo sapiens [sequence omitted]

SEQ ID NO: 363 - protein, 1122 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMKTVLMVAEKPSLAQSIAKILSRGSLSSHKGLNGACSVHEYTGTFAGQPVRFKMTSVCGHVMTLDFLGKYNKWDKVDPAELFSQAPTEKKEANPKLNMVKFLQVEGRGCDYIVLWLDCDKEGENICFEVLDAVLPVMNKAHGGEKTVFRARFSSITDTDICNAMACLGEPDHNEALSVDARQELDLRIGCAFTRFQTKYFQGKYGDLDSSLISFGPCQTPTLGFCVERHDKIQSFKPETYWVLQAKVNTDKDRSLLLDWDRVRVFDREIAQMFLNMTKLEKEAQVEATSRKEKAKQRPLALNTVEMLRVASSSLGMGPQHAMQTAERLYTQGYISYPRTETTHYPENFDLKGSLRQQANHPYWADTVKRLLAEGINRPRKGHDAGDHPPITPMKSATEAELGGDAWRLYEYITRHFIATVSHDCKYLQSTISFRIGPELFTCSGKTVLSPGFTEVMPWQSVPLEESLPTCQRGDAFPVGEVKMLEKQTNPPDYLTEAELITLMEKHGIGTDASIPVHINNICQRNYVTVESGRRLKPTNLGIVLVHGYYKIDAELVLPTIRSAVEKQLNLIAQGKADYRQVLGHTLDVFKRKFHYFVDSIAGMDELMEVSFSPLAATGKPLSRCGKCHRFMKYIQAKPSRLHCSHCDETYTLPQNGTIKLYKELRCPLDDFELVLWSSGSRGKSYPLCPYCYNHPPFRDMKKGMGCNECTHPSCQHSLSMLGIGQCVECESGVLVLDPTSGPKWKVACNKCNVVAHCFENAHRVRVSADTCSVCEAALLDVDFNKAKSPLPGDETQHMGCVFCDPVFQELVELKHAASCHPMHRGGPGRRQGRGRGRARRPPGKPNPRRPKDKMSALAAYFV

SEQ ID NO: 364 - protein, 862 aa, Homo sapiens
MKTVLMVAEKPSLAQSIAKILSRGSLSSHKGLNGACSVHEYTGTFAGQPVRFKMTSVCGHVMTLDFLGKYNKWDKVDPAELFSQAPTEKKEANPKLNMVKFLQVEGRGCDYIVLWLDCDKEGENICFEVLDAVLPVMNKAHGGEKTVFRARFSSITDTDICNAMACLGEPDHNEALSVDARQELDLRIGCAFTRFQTKYFQGKYGDLDSSLISFGPCQTPTLGFCVERHDKIQSFKPETYWVLQAKVNTDKDRSLLLDWDRVRVFDREIAQMFLNMTKLEKEAQVEATSRKEKAKQRPLALNTVEMLRVASSSLGMGPQHAMQTAERLYTQGYISYPRTETTHYPENFDLKGSLRQQANHPYWADTVKRLLAEGINRPRKGHDAGDHPPITPMKSATEAELGGDAWRLYEYITRHFIATVSHDCKYLQSTISFRIGPELFTCSGKTVLSPGFTEVMPWQSVPLEESLPTCQRGDAFPVGEVKMLEKQTNPPDYLTEAELITLMEKHGIGTDASIPVHINNICQRNYVTVESGRRLKPTNLGIVLVHGYYKIDAELVLPTIRSAVEKQLNLIAQGKADYRQVLGHTLDVFKRKFHYFVDSIAGMDELMEVSFSPLAATGKPLSRCGKCHRFMKYIQAKPSRLHCSHCDETYTLPQNGTIKLYKELRCPLDDFELVLWSSGSRGKSYPLCPYCYNHPPFRDMKKGMGCNECTHPSCQHSLSMLGIGQCVECESGVLVLDPTSGPKWKVACNKCNVVAHCFENAHRVRVSADTCSVCEAALLDVDFNKAKSPLPGDETQHMGCVFCDPVFQELVELKHAASCHPMHRGGPGRRQGRGRGRARRPPGKPNPRRPKDKMSALAAYFV

SEQ ID NO: 365 - protein, 663 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTRAEWQVAEATALVHTLDGWSVVQTMVVSTKTPDRKLIFGKGNFEHLTEKIRGSPDVTCVFLNVERMAAPTKKELEAAWGVEVFDRFTVVLHIFRCNARTKEARLQVALAEMPLHRSNLKRDVAHLYRGVGSRYIMGSGESFMQLQQRLLREKEAKIRKALDRLRKKRHLLRRQRTRREFPVISVVGYTNCGKTTLIKALTGDAAIQPRDQLFATLDVTAHAGTLPSRMTVLYVDTIGFLSQLPHGLIESFSATLEDVAHSDLILHVRDVSHPEAELQKCSVLSTLRGLQLPAPLLDSMVEVHNKVDLVPGYSPTEPNVVPVSALRGHGLQELKAELDAAVLKATGRQILTLRVRLAGAQLSWLYKEATVQEVDVIPEDGAADVRVIISNSAYGKFRKLFPG

SEQ ID NO: 366 - protein, 403 aa, Homo sapiens
MTRAEWQVAEATALVHTLDGWSVVQTMVVSTKTPDRKLIFGKGNFEHLTEKIRGSPDVTCVFLNVERMAAPTKKELEAAWGVEVFDRFTVVLHIFRCNARTKEARLQVALAEMPLHRSNLKRDVAHLYRGVGSRYIMGSGESFMQLQQRLLREKEAKIRKALDRLRKKRHLLRRQRTRREFPVISVVGYTNCGKTTLIKALTGDAAIQPRDQLFATLDVTAHAGTLPSRMTVLYVDTIGFLSQLPHGLIESFSATLEDVAHSDLILHVRDVSHPEAELQKCSVLSTLRGLQLPAPLLDSMVEVHNKVDLVPGYSPTEPNVVPVSALRGHGLQELKAELDAAVLKATGRQILTLRVRLAGAQLSWLYKEATVQEVDVIPEDGAADVRVIISNSAYGKFRKLFPG

SEQ ID NO: 367 - protein, 919 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPGPPRSLEMGLLTFRDVAIEFSLEEWQHLDIAQQNLYRNVMLENYRNLAFLGIAVSKPDLITCLEQGKEPWNMKRHEMVDEPPGMCPHFAQGLWPEQGMEDSFQKAILRRYGKYGHENLQLRKGCKSVDEYKVNKEGYNGLNQCFTTAQSKVFQCDKYLKVFYKFLNSNRPKIRHTEKKSFKCKKRVKLFCMLSHKTQHKSIYHREKSYKCKECGKTFNWSSTLTNHRKIYTEEKPYKCEEYNKSPKQLSTLTTHEIIHAGEKLYKCEECGEAFNRSSNLTTHKIIHTGEKPYKCEECGKAFIWSSTLTEHKKIHTRKKPYKCEECGKAFIWSSTLTRHKRMHTGEKPYKCEECGKAFSQSSTLTTHKIIHTGEKRYKCLECGKAFKQLSTLTTHKIIHVGEKLYKCEECGKGFNRSSNLTTHKIIHTGEKPYKCEECGKAFIWSSTLTKHKRIHTREKPYKCEECGKAFIWSSTLTRHKRMHTGEKPYKCEECGKSFSQSSTLTTHKIIHTGEKPYKCEECGKAFNWSSTLTKHNIIHTEEKPYKCEKCGKAFKQSSILTNHKRIHTGEKPYKCEECGKSFNRSSTFTKHKVIHTGVKPYKCEECGKAFFWSSTLTKHKRIHTGEQPYKWEKFGKAFNRSSHLTTDKITHWREILQV

SEQ ID NO: 368 - protein, 659 aa, Homo sapiens
MPGPPRSLEMGLLTFRDVAIEFSLEEWQHLDIAQQNLYRNVMLENYRNLAFLGIAVSKPDLITCLEQGKEPWNMKRHEMVDEPPGMCPHFAQGLWPEQGMEDSFQKAILRRYGKYGHENLQLRKGCKSVDEYKVNKEGYNGLNQCFTTAQSKVFQCDKYLKVFYKFLNSNRPKIRHTEKKSFKCKKRVKLFCMLSHKTQHKSIYHREKSYKCKECGKTFNWSSTLTNHRKIYTEEKPYKCEEYNKSPKQLSTLTTHEIIHAGEKLYKCEECGEAFNRSSNLTTHKIIHTGEKPYKCEECGKAFIWSSTLTEHKKIHTRKKPYKCEECGKAFIWSSTLTRHKRMHTGEKPYKCEECGKAFSQSSTLTTHKIIHTGEKRYKCLECGKAFKQLSTLTTHKIIHVGEKLYKCEECGKGFNRSSNLTTHKIIHTGEKPYKCEECGKAFIWSSTLTKHKRIHTREKPYKCEECGKAFIWSSTLTRHKRMHTGEKPYKCEECGKSFSQSSTLTTHKIIHTGEKPYKCEECGKAFNWSSTLTKHNIIHTEEKPYKCEKCGKAFKQSSILTNHKRIHTGEKPYKCEECGKSFNRSSTFTKHKVIHTGVKPYKCEECGKAFFWSSTLTKHKRIHTGEQPYKWEKFGKAFNRSSHLTTDKITHWREILQV

SEQ ID NO: 369 - protein, 999 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEPKPATKKATRTKAISEDLSQEAILEKLTENGLWDSRMEGLWKWNDRILRLQNNQENHLSQRIIPLKKTPTSQRGFRFESILIPEPGIATEELHSRCQTQEENFTENLNLITDTHLGKIICKEMKGSKAIRQTSELTLGKKSNNKEKPYKCSTCEKAFHYRSLLIQHQRTHTKEKPYECNECGKTFSQPSYLSQHKKIHTGEKPYKCNECGKAFIASSSLMVHQRIHTKEKPYQCNVCGKSFSQCARLNQHQRIQTGEKPYKCSECGKAFSDKSKLARHQETHNGEKPYKCDDCGKAFRNKSYLSVHQKTHTEEKPYQCNECGKSFKNTTIFNVHQRIHTGEKPFRCNECGKAYRSNSSLIVHIRTHTGEKPYECNECGKAFNRIANFTEHQRIHTGEKPYKCNECGKAFINYSCLTVHHRMHTGEKPYKCTECGKAFMRSSSLIIHQRIHTEEKPYLCNECGESFRIKSHLTVHQRIHTGEKPYKCTDCERAFTKMVNLKEHQKIHTGVKPYKCYDCGKSFRTKSYLIVHQRTHTGEKPYKCNECEKAFTNTSQLTVHQRRHTGEKPYKCNECGKVFTSNSGFNTHQRTHTGEKPFKCNDCGKAFSQMVHVTEHQKIHTGEKPYKCDVCGKAFRRGSYLTVHWRTHTGEKPYTCKECGKGCITLSQLTLHQRIHTGERPYKCEECGKAFRTNSDFTVHLRMHTGEKPYKCNECGKAFRSSSSLTVHQRIHQRETQLI

SEQ ID NO: 370 - protein, 739 aa, Homo sapiens
MEPKPATKKATRTKAISEDLSQEAILEKLTENGLWDSRMEGLWKWNDRILRLQNNQENHLSQRIIPLKKTPTSQRGFRFESILIPEPGIATEELHSRCQTQEENFTENLNLITDTHLGKIICKEMKGSKAIRQTSELTLGKKSNNKEKPYKCSTCEKAFHYRSLLIQHQRTHTKEKPYECNECGKTFSQPSYLSQHKKIHTGEKPYKCNECGKAFIASSSLMVHQRIHTKEKPYQCNVCGKSFSQCARLNQHQRIQTGEKPYKCSECGKAFSDKSKLARHQETHNGEKPYKCDDCGKAFRNKSYLSVHQKTHTEEKPYQCNECGKSFKNTTIFNVHQRIHTGEKPFRCNECGKAYRSNSSLIVHIRTHTGEKPYECNECGKAFNRIANFTEHQRIHTGEKPYKCNECGKAFINYSCLTVHHRMHTGEKPYKCTECGKAFMRSSSLIIHQRIHTEEKPYLCNECGESFRIKSHLTVHQRIHTGEKPYKCTDCERAFTKMVNLKEHQKIHTGVKPYKCYDCGKSFRTKSYLIVHQRTHTGEKPYKCNECEKAFTNTSQLTVHQRRHTGEKPYKCNECGKVFTSNSGFNTHQRTHTGEKPFKCNDCGKAFSQMVHVTEHQKIHTGEKPYKCDVCGKAFRRGSYLTVHWRTHTGEKPYTCKECGKGCITLSQLTLHQRIHTGERPYKCEECGKAFRTNSDFTVHLRMHTGEKPYKCNECGKAFRSSSSLTVHQRIHQRETQLI

SEQ ID NO: 371 - protein, 618 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAPIPKTVGRIKLDCSLRPSCPLEVAAAPKLCKEFGPEDYGEEDIVDFLRRLVESDPQGLHRIHVDGSSGRLQLWHHDYLLGHLDDEGKSTGQSDRGKGAEGLGTYCGLRKSFLYPPQESEPCPQSPSASATFPSVSDSLLQVAMPQKLLVTEEEANRLAEELVAEEERMKQKAEKKRLKKKRQKERKRQERLEQYCGEPKASTTSDGDESPPSSPGNPVQGQCGEEEDSLDLSSTFVSLALRKVGDWPLSARREKGLNQEPQGRGLALQKMGQEEESPPREERPQQSPKEKDLGRLRPQDLLDFAPYPQASPGLLAAALQQSQELAKLGTSFAQNGFYHEAVVLFTQALKLNPQDHR

SEQ ID NO: 372 - protein, 358 aa, Homo sapiens
MAPIPKTVGRIKLDCSLRPSCPLEVAAAPKLCKEFGPEDYGEEDIVDFLRRLVESDPQGLHRIHVDGSSGRLQLWHHDYLLGHLDDEGKSTGQSDRGKGAEGLGTYCGLRKSFLYPPQESEPCPQSPSASATFPSVSDSLLQVAMPQKLLVTEEEANRLAEELVAEEERMKQKAEKKRLKKKRQKERKRQERLEQYCGEPKASTTSDGDESPPSSPGNPVQGQCGEEEDSLDLSSTFVSLALRKVGDWPLSARREKGLNQEPQGRGLALQKMGQEEESPPREERPQQSPKEKDLGRLRPQDLLDFAPYPQASPGLLAAALQQSQELAKLGTSFAQNGFYHEAVVLFTQALKLNPQDHR

SEQ ID NO: 373 - protein, 585 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSLLRSLRVFLVARTGSYPAGSLLRQSPQPRHTFYAGPRLSASASSKELLMKLRRKTGYSFVNCKKALETCGGDLKQAEIWLHKEAQKEGWSKAAKLQGRKTKEGLIGLLQEGNTTVLVEVNCETDFVSRNLKFQLLVQQVALGTMMHCQTLKDQPSAYSKGFLNSSELSGLPAGPDREGSLKDQLALAIGKLGETMILKRAAWVKVPSGFYVGSYVHGAMQSPSLHKLVLGKYGALVICETSEQKTNLEDVGRRLGQHVVGMAPLSVGSLDDEPGGEAETKMLSQPYLLDPSITLGQYVQPQGVSVVDFVRFECGEGEEAAETE

SEQ ID NO: 374 - protein, 325 aa, Homo sapiens
MSLLRSLRVFLVARTGSYPAGSLLRQSPQPRHTFYAGPRLSASASSKELLMKLRRKTGYSFVNCKKALETCGGDLKQAEIWLHKEAQKEGWSKAAKLQGRKTKEGLIGLLQEGNTTVLVEVNCETDFVSRNLKFQLLVQQVALGTMMHCQTLKDQPSAYSKGFLNSSELSGLPAGPDREGSLKDQLALAIGKLGETMILKRAAWVKVPSGFYVGSYVHGAMQSPSLHKLVLGKYGALVICETSEQKTNLEDVGRRLGQHVVGMAPLSVGSLDDEPGGEAETKMLSQPYLLDPSITLGQYVQPQGVSVVDFVRFECGEGEEAAETE

SEQ ID NO: 375 - protein, 453 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDNLRETFLSLEDGLGSSDSPGLLSSWDWKDRAGPFELNQASPSQSLSPAPSLESYSSSPCPAVAGLPCEHGGASSGGSEGCSVGGASGLVEVDYNMLAFQPTHLQGGGGPKAQKGTKVRMSVQRRRKASEREKLRMRTLADALHTLRNYLPPVYSQRGQPLTKIQTLKYTIKYIGELTDLLNRGREPRAQSA

SEQ ID NO: 376 - protein, 193 aa, Homo sapiens
MDNLRETFLSLEDGLGSSDSPGLLSSWDWKDRAGPFELNQASPSQSLSPAPSLESYSSSPCPAVAGLPCEHGGASSGGSEGCSVGGASGLVEVDYNMLAFQPTHLQGGGGPKAQKGTKVRMSVQRRRKASEREKLRMRTLADALHTLRNYLPPVYSQRGQPLTKIQTLKYTIKYIGELTDLLNRGREPRAQSA

SEQ ID NO: 377 - protein, 742 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVILRDKIRFYEELQRDKAAAAAVLGAVRKRPSVVPMAGQDPALSTSHPFYDVARHGILQVAGDDRFGRRVVTFSCCRMPPSHELDHQRLLEYLKYTLDQYVENDYTIVYFHYGLNSRNKPSLGWLQSAYKEFDRKYKKNLKALYVVHPTSFIKVLWNILKPLISHKFGKKVIYFNYLSELHEHLKYDQLVIPPEVLRYDEKLQSLHEGRTPPPTKTPPPRPPLPTQQFGVSLQYLKDKNQGELIPPVLRFTVTYLREKGLRTEGLFRRSASVQTVREIQRLYNQGKPVNFDDYGDIHIPAVILKTFLRELPQPLLTFQAYEQILGITCVESSLRVTGCRQILRSLPEHNYVVLRYLMGFLHAVSRESIFNKMNSSNLACVFGLNLIWPSQGVSSLSALVPLNMFTELLIEYYEKIFSTPEAPGEHGLAPWEQGSRAAPLQEAVPRTQATGLTKPTLPPSPLMAARRRLNPAFLYKVVKASS

SEQ ID NO: 378 - protein, 469 aa, Homo sapiens
MVILRDKIRFYEELQRDKAAAAAVLGAVRKRPSVVPMAGQDPALSTSHPFYDVARHGILQVAGDDRFGRRVVTFSCCRMPPSHELDHQRLLEYLKYTLDQYVENDYTIVYFHYGLNSRNKPSLGWLQSAYKEFDRKYKKNLKALYVVHPTSFIKVLWNILKPLISHKFGKKVIYFNYLSELHEHLKYDQLVIPPEVLRYDEKLQSLHEGRTPPPTKTPPPRPPLPTQQFGVSLQYLKDKNQGELIPPVLRFTVTYLREKGLRTEGLFRRSASVQTVREIQRLYNQGKPVNFDDYGDIHIPAVILKTFLRELPQPLLTFQAYEQILGITCVESSLRVTGCRQILRSLPEHNYVVLRYLMGFLHAVSRESIFNKMNSSNLACVFGLNLIWPSQGVSSLSALVPLNMFTELLIEYYEKIFSTPEAPGEHGLAPWEQGSRAAPLQEAVPRTQATGLTKPTLPPSPLMAARRRL

SEQ ID NO: 379 - protein, 690 aa, synthetic construct; identical to SEQ ID NO: 221

SEQ ID NO: 380 - protein, 417 aa, Homo sapiens; identical to SEQ ID NO: 222

SEQ ID NO: 381 - protein, 961 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSSRTVLAPGNDRNSDTHGTLGSGRSSDKGPSWSSRSLGARCRNSIASCPEEQPHVGNYRLLRTIGKGNFAKVKLARHILTGREVAIKIIDKTQLNPSSLQKLFREVRIMKGLNHPNIVKLFEVIETEKTLYLVMEYASAGEVFDYLVSHGRMKEKEARAKFRQIVSAVHYCHQKNIVHRDLKAENLLLDAEANIKIADFGFSNEFTLGSKLDTFCGSPPYAAPELFQGKKYDGPEVDIWSLGVILYTLVSGSLPFDGHNLKELRERVLRGKYRVPFYMSTDCESILRRFLVLNPAKRCTLEQIMKDKWINIGYEGEELKPYTEPEEDFGDTKRIEVMVGMGYTREEIKESLTSQKYNEVTATYLLLGRKTEEGGDRGAPGLALARVRAPSDTTNGTSSSKGTSHSKGQRSSSSTYHRQRRHSDFCGPSPAPLHPKRSPTSTGEAELKEERLPGRKASCSTAGSGSRGLPPSSPMVSSAHNPNKAEIPERRKDSTSTPNNLPPSMMTRRNTYVCTERPGAERPSLLPNGKENSSGTPRVPPASPSSHSLAPPSGERSRLARGSTIRSTFHGGQVRDRRAGGGGGGGVQNGPPASPTLAHEAAPLPAGRPRPTTNLFTKLTSKLTRRVTLDPSKRQNSNRCVSGASLPQGSKIRSQTNLRESGDLRSQVAIYLGIKRKPPPGCSDSPGVNPAFLYKVVKASS

SEQ ID NO: 382 - protein, 688 aa, Homo sapiens
MSSRTVLAPGNDRNSDTHGTLGSGRSSDKGPSWSSRSLGARCRNSIASCPEEQPHVGNYRLLRTIGKGNFAKVKLARHILTGREVAIKIIDKTQLNPSSLQKLFREVRIMKGLNHPNIVKLFEVIETEKTLYLVMEYASAGEVFDYLVSHGRMKEKEARAKFRQIVSAVHYCHQKNIVHRDLKAENLLLDAEANIKIADFGFSNEFTLGSKLDTFCGSPPYAAPELFQGKKYDGPEVDIWSLGVILYTLVSGSLPFDGHNLKELRERVLRGKYRVPFYMSTDCESILRRFLVLNPAKRCTLEQIMKDKWINIGYEGEELKPYTEPEEDFGDTKRIEVMVGMGYTREEIKESLTSQKYNEVTATYLLLGRKTEEGGDRGAPGLALARVRAPSDTTNGTSSSKGTSHSKGQRSSSSTYHRQRRHSDFCGPSPAPLHPKRSPTSTGEAELKEERLPGRKASCSTAGSGSRGLPPSSPMVSSAHNPNKAEIPERRKDSTSTPNNLPPSMMTRRNTYVCTERPGAERPSLLPNGKENSSGTPRVPPASPSSHSLAPPSGERSRLARGSTIRSTFHGGQVRDRRAGGGGGGGVQNGPPASPTLAHEAAPLPAGRPRPTTNLFTKLTSKLTRRVTLDPSKRQNSNRCVSGASLPQGSKIRSQTNLRESGDLRSQVAIYLGIKRKPPPGCSDSPGV

SEQ ID NO: 383 - protein, 1015 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEEGFRDRAAFIRGAKDIAKEVKKHAAKKVVKGLDRVQDEYSRRSYSRFEEEDDDDDFPAPSDGYYRGEGTQDEEEGGASSDATEGHDEDDEIYEGEYQDIPRAESGGKGERMADGAPLAGVRGGLSDGEGPPGGRGEAQRRKEREELAQQYEAILRECGHGRFQWTLYFVLGLALMADGVEVFVVGFVLPSAEKDMCLSDSNKGMLGLIVYLGMMVGAFLWGGLADRLGRRQCLLISLSVNSVFAFFSSFVQGYGTFLFCRLLSGVGIGGSIPIVFSYFSEFLAQEKRGEHLSWLCMFWMIGGVYAAAMAWAIIPHYGWSFQMGSAYQFHSWRVFVLVCAFPSVFAIGALTTQPESPRFFLENGKHDEAWMVLKQVHDTNMRAKGHPERVFSVTHIKTIHQEDELIEIQSDTGTWYQRWGVRALSLGGQVWGNFLSCFGPEYRRITLMMMGVWFTMSFSYYGLTVWFPDMIRHLQAVDCASRTKVFPGERVGHVTFNFTLENQIHRGGQYFNDKFIGLRLKSVSFEDSLFEECYFEDVTSSNTFFRNCTFINTVFYNTDLFEYKFVNSRLINSTFLHNKEGCPLDVTGTGEGAYMVYFVSFLGTLAVLPGNIVSALLMDKIGRLRMLAGSSVMSCVSCFFLSFGNSESAMIALLCLFGGVSIASWNALDVLTVELYPSDKRTTAFGFLNALCKLAAVLGISIFTSFVGITKAAPILFASAALALGSSLALKLPETRGQVLQNPAFLYKVVKASS

SEQ ID NO: 384 - protein, 742 aa, Homo sapiens
MEEGFRDRAAFIRGAKDIAKEVKKHAAKKVVKGLDRVQDEYSRRSYSRFEEEDDDDDFPAPSDGYYRGEGTQDEEEGGASSDATEGHDEDDEIYEGEYQDIPRAESGGKGERMADGAPLAGVRGGLSDGEGPPGGRGEAQRRKEREELAQQYEAILRECGHGRFQWTLYFVLGLALMADGVEVFVVGFVLPSAEKDMCLSDSNKGMLGLIVYLGMMVGAFLWGGLADRLGRRQCLLISLSVNSVFAFFSSFVQGYGTFLFCRLLSGVGIGGSIPIVFSYFSEFLAQEKRGEHLSWLCMFWMIGGVYAAAMAWAIIPHYGWSFQMGSAYQFHSWRVFVLVCAFPSVFAIGALTTQPESPRFFLENGKHDEAWMVLKQVHDTNMRAKGHPERVFSVTHIKTIHQEDELIEIQSDTGTWYQRWGVRALSLGGQVWGNFLSCFGPEYRRITLMMMGVWFTMSFSYYGLTVWFPDMIRHLQAVDCASRTKVFPGERVGHVTFNFTLENQIHRGGQYFNDKFIGLRLKSVSFEDSLFEECYFEDVTSSNTFFRNCTFINTVFYNTDLFEYKFVNSRLINSTFLHNKEGCPLDVTGTGEGAYMVYFVSFLGTLAVLPGNIVSALLMDKIGRLRMLAGSSVMSCVSCFFLSFGNSESAMIALLCLFGGVSIASWNALDVLTVELYPSDKRTTAFGFLNALCKLAAVLGISIFTSFVGITKAAPILFASAALALGSSLALKLPETRGQVLQ

SEQ ID NO: 385 - protein, 1133 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMMSIKAFTLVSAVERELLMGDKERVNIECVECCGRDLYVGTNDCFVYHFLLEERPVPAGPATFTATKQLQRHLGFKKPVNELRAASALNRLLVLCDNSISLVNMLNLEPVPSGARIKGAATFALNENPVSGDPFCVEVCIISVKRRTIQMFLVYEDRVQIVKEVSTAEQPLAVAVDGHFLCLALTTQYIIHNYSTGVSQDLFPYCSEERPPIVKRIGRQEFLLAGPGGLGMFATVAGISQRAPVHWSENVIGAAVSFPYVIALDDEFITVHSMLDQQQKQTLPFKEGHILQDFEGRVIVATSKGVYILVPLPLEKQIQDLLASRRVEEALVLAKGARRNIPKEKFQVMYRRILQQAGFIQFAQLQFLEAKELFRSGQLDVRELISLYPFLLPTSSSFTRSHPPLHEYADLNQLTQGDQEKMAKCKRFLMSYLNEVRSTEVANGYKEDIDTALLKLYAEADHDSLLDLLVTENFCLLTDSAAWLEKHKKYFALGLLYHYNNQDAAAVQLWVNIVNGDVQDSTRSDLYEYIVDFLTYCLDEELVWAYADWVLQKSEEVGVQVFTKRPLDEQQKNSFNPDDIINCLKKYPKALVKYLEHLVIDKRLQKEEYHTHLAVLYLEEVLLQRASASGKGAEATETQAKLRRLLQKSDLYRVHFLLERLQGAGLPMESAILHGKLGEHEKALHILVHELQDFAAAEDYCLWCSEGRDPPHRQQLFHTLLAIYLHAGPTAHELAVAAVDLLNRHATEFDAAQVLQMLPDTWSVQLLCPFLMGAMRDSIHARRTMQVALGLARSENLIYTYDKMKLKGSSIQLSDKKLCQICQNPFCEPVFVRYPNGGLVHTHCAASRHTNPSSSSPGTRTNPAFLYKVVKASS

SEQ ID NO: 386 - protein, 860 aa, Homo sapiens
MMSIKAFTLVSAVERELLMGDKERVNIECVECCGRDLYVGTNDCFVYHFLLEERPVPAGPATFTATKQLQRHLGFKKPVNELRAASALNRLLVLCDNSISLVNMLNLEPVPSGARIKGAATFALNENPVSGDPFCVEVCIISVKRRTIQMFLVYEDRVQIVKEVSTAEQPLAVAVDGHFLCLALTTQYIIHNYSTGVSQDLFPYCSEERPPIVKRIGRQEFLLAGPGGLGMFATVAGISQRAPVHWSENVIGAAVSFPYVIALDDEFITVHSMLDQQQKQTLPFKEGHILQDFEGRVIVATSKGVYILVPLPLEKQIQDLLASRRVEEALVLAKGARRNIPKEKFQVMYRRILQQAGFIQFAQLQFLEAKELFRSGQLDVRELISLYPFLLPTSSSFTRSHPPLHEYADLNQLTQGDQEKMAKCKRFLMSYLNEVRSTEVANGYKEDIDTALLKLYAEADHDSLLDLLVTENFCLLTDSAAWLEKHKKYFALGLLYHYNNQDAAAVQLWVNIVNGDVQDSTRSDLYEYIVDFLTYCLDEELVWAYADWVLQKSEEVGVQVFTKRPLDEQQKNSFNPDDIINCLKKYPKALVKYLEHLVIDKRLQKEEYHTHLAVLYLEEVLLQRASASGKGAEATETQAKLRRLLQKSDLYRVHFLLERLQGAGLPMESAILHGKLGEHEKALHILVHELQDFAAAEDYCLWCSEGRDPPHRQQLFHTLLAIYLHAGPTAHELAVAAVDLLNRHATEFDAAQVLQMLPDTWSVQLLCPFLMGAMRDSIHARRTMQVALGLARSENLIYTYDKMKLKGSSIQLSDKKLCQICQNPFCEPVFVRYPNGGLVHTHCAASRHTNPSSSSPGTRT

SEQ ID NO: 387 - protein, 360 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMRLHLLLLILLLFSILLSPVRGGLGPAEGHCLNLFGVCRTDVCNIVEDQIGACRRRMKCCRAWWILMPIPTPLIMSDYQEPLKPNLKNPAFLYKVVKASS

SEQ ID NO: 388 - protein, 87 aa, Homo sapiens
MRLHLLLLILLLFSILLSPVRGGLGPAEGHCLNLFGVCRTDVCNIVEDQIGACRRRMKCCRAWWILMPIPTPLIMSDYQEPLKPNLK

SEQ ID NO: 389 - protein, 370 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDKQSSAGGVKRSVPCDSNEANEMMPETPTGDSDPQPAPKKMKTSESSTILVVRYRRNFKRTSPEELLNDHARENRINPLQMEEEEFMEIMVEIPAKNPAFLYKVVKASS

SEQ ID NO: 390 - protein, 97 aa, Homo sapiens
MDKQSSAGGVKRSVPCDSNEANEMMPETPTGDSDPQPAPKKMKTSESSTILVVRYRRNFKRTSPEELLNDHARENRINPLQMEEEEFMEIMVEIPAK

SEQ ID NO: 391 - protein, 763 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMALRAKAEVCVAAPWLSLQRARALGTRAARAPRTVLPFEAMPQHPGNRWLRLLQIWREQGYEHLHLEMHQTFQELGPIFRYNLGGPRMVCVMLPEDVEKLQQVDSLHPCRMILEPWVAYRQHRGHKCGVFLLNGPEWRFNRLRLNPDVLSPKAVQRFLPMVDAVARDFSQALKKKVLQNARGSLTLDVQPSIFHYTIEASNLALFGERLGLVGHSPSSASLNFLHALEVMFKSTVQLMFMPRSLSRWISPKVWKEHFEAWDCIFQYGDNCIQKIYQELAFNRPQHYTGIVAELLLKAELSLEAIKANSMELTAGSVDTTAFPLLMTLFELARNPDVQQILRQESLAAAASISEHPQKATTELPLLRAALKETLRLYPVGLFLERVVSSDLVLQNYHIPAGTLVQVFLYSLGRNAALFPRPERYNPQRWLDIRGSGRNFHHVPFGFGMRQCLGRRLAEAEMLLLLHHVLKHFLVETLTQEDIKMVYSFILRPGTSPLLTFRAIN

SEQ ID NO: 392 - protein, 503 aa, Homo sapiens
MALRAKAEVCVAAPWLSLQRARALGTRAARAPRTVLPFEAMPQHPGNRWLRLLQIWREQGYEHLHLEMHQTFQELGPIFRYNLGGPRMVCVMLPEDVEKLQQVDSLHPCRMILEPWVAYRQHRGHKCGVFLLNGPEWRFNRLRLNPDVLSPKAVQRFLPMVDAVARDFSQALKKKVLQNARGSLTLDVQPSIFHYTIEASNLALFGERLGLVGHSPSSASLNFLHALEVMFKSTVQLMFMPRSLSRWISPKVWKEHFEAWDCIFQYGDNCIQKIYQELAFNRPQHYTGIVAELLLKAELSLEAIKANSMELTAGSVDTTAFPLLMTLFELARNPDVQQILRQESLAAAASISEHPQKATTELPLLRAALKETLRLYPVGLFLERVVSSDLVLQNYHIPAGTLVQVFLYSLGRNAALFPRPERYNPQRWLDIRGSGRNFHHVPFGFGMRQCLGRRLAEAEMLLLLHHVLKHFLVETLTQEDIKMVYSFILRPGTSPLLTFRAIN

SEQ ID NO: 393 - protein, 1024 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 394 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 395 - protein, 748 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 396 - protein, 488 aa, Homo sapiens
MAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 397 - protein, 493 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGNVQERPSETIDRERKRLVETLQADSGLLLDALVARGVLTGPEYEALDALPDAERRVRRLLLLVQSKGEAACQELLRCAQQTVRMPDPAWDWQHVGPGYRNRSYDPSCPGHWTPEAPSSGTTCPELPRASEQEEVGGPEGSEALQPRTPEEPELEAEATEGDEPDLEQEMNPEQEPEPEPEPEPEPEPEPEPEPEPEPEPEPEPEPEPDFQEEDEFEDSNPAFLYKVVKASS

SEQ ID NO: 398 - protein, 220 aa, Homo sapiens
MGNVQERPSETIDRERKRLVETLQADSGLLLDALVARGVLTGPEYEALDALPDAERRVRRLLLLVQSKGEAACQELLRCAQQTVRMPDPAWDWQHVGPGYRNRSYDPSCPGHWTPEAPSSGTTCPELPRASEQEEVGGPEGSEALQPRTPEEPELEAEATEGDEPDLEQEMNPEQEPEPEPEPEPEPEPEPEPEPEPEPEPEPEPEPEPDFQEEDEFEDS

SEQ ID NO: 399 - protein, 917 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEETMKLATMEDTVEYCLFLIPDESRDSDKHKEILQKYIERIITRFAPMLVPYIWQNQPFNLKYKPGKGGVPAHMFGVTKFGDNIEDEWFIVYVIKQITKEFPELVARIEDNDGEFLLIEAADFLPKWLDPENSTNRVFFCHGELCIIPAPRKSGAESWLPTTPPTIPQALNIITAHSEKILASESIRAAVNRRIRGYPEKIQASLHRAHCFLPAGIVAVLKQRPRLVAAAVQAFYLRDPIDLRACRVFKTFLPETRIMTSVTFTKCLYAQLVQQRFVPDRRSGYRLPPPSDPQYRAHELGMKLAHGFEILCSKCSPHFSDCKKSLVTASPLWASFLESLKKNDYFKGLIEGSAQYRERLEMAENYFQLSVDWPESSLAMSPGEEILTLLQTIPFDIEDLKKEAANLPPEDDDQWLDLSPDQLDQLLQEAVGKKESESVSKEEKEQNYDLTEVSESMKAFISKVSTHKGAELPREPSEAPITFDADSFLNYFDKILGPRPNESDSDDLDDEDFECLDSDDDLDFETHEPGEEASLKGTLDNLKSYMAQMDQELAHTCISKSFTTRNQVEPVSQTTDNNSDEEDSGTGESVMAPVDVDLNLVSNILESYSSQAGLAGPASNLLQSMGVQLPDNTDHRPTSKPTKNNPAFLYKVVKASS

SEQ ID NO: 400 - protein, 644 aa, Homo sapiens
MEETMKLATMEDTVEYCLFLIPDESRDSDKHKEILQKYIERIITRFAPMLVPYIWQNQPFNLKYKPGKGGVPAHMFGVTKFGDNIEDEWFIVYVIKQITKEFPELVARIEDNDGEFLLIEAADFLPKWLDPENSTNRVFFCHGELCIIPAPRKSGAESWLPTTPPTIPQALNIITAHSEKILASESIRAAVNRRIRGYPEKIQASLHRAHCFLPAGIVAVLKQRPRLVAAAVQAFYLRDPIDLRACRVFKTFLPETRIMTSVTFTKCLYAQLVQQRFVPDRRSGYRLPPPSDPQYRAHELGMKLAHGFEILCSKCSPHFSDCKKSLVTASPLWASFLESLKKNDYFKGLIEGSAQYRERLEMAENYFQLSVDWPESSLAMSPGEEILTLLQTIPFDIEDLKKEAANLPPEDDDQWLDLSPDQLDQLLQEAVGKKESESVSKEEKEQNYDLTEVSESMKAFISKVSTHKGAELPREPSEAPITFDADSFLNYFDKILGPRPNESDSDDLDDEDFECLDSDDDLDFETHEPGEEASLKGTLDNLKSYMAQMDQELAHTCISKSFTTRNQVEPVSQTTDNNSDEEDSGTGESVMAPVDVDLNLVSNILESYSSQAGLAGPASNLLQSMGVQLPDNTDHRPTSKPTKN

SEQ ID NO: 401 - protein, 1000 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEADGDGEELARLRSVFAACDANRSGRLEREEFRALCTELRVRPADAEAVFQRLDADRDGAITFQEFARGFLGSLRGGRRRDWGPLDPAPAVSEAGPETHDSEEDEGDEDAAAALATSCGPASPGRAWQDFQARLGDEAKFIPREEQVSTLYQNINLVEPRLIQPYEHVIKNFIREIRLQSTEMENLAIAVKRAQDKAAMQLSELEEEMDQRIQAAEHKTRKDEKRKAEEALSDLRRQYETEVGDLQVTIKKLRKLEEQSKRVSQKEDVAALKKQIYDLSMENQKVKKDLLEAQTNIAFLQSELDALKSDYADQSLNTERDLEIIRAYTEDRNSLERQIEILQTANRKLHDSNDGLRSALENSYSKFNRSLHINNISPGNTISRSSPKFIGHSPQPLGYDRSSRSSYVDEDCDSLALCDPLQRTNCEVDSLPESCFDSGLSTLRDPNEYDSEVEYKHQRGFQRSHGVQESFGGDASDTDVPDIRDEETFGLEDVASVLDWKPQGSVSEGSIVSSSRKPISALSPQTDLVDDNAKSFSSQKAYKIVLAGDAAVGKSSFLMRLCKNEFRENISATLGVDFQMKTLIVDGERTVLQLWDTAGQERFRSIAKSYFRKADGVLLLYDVTCEKSFLNIREWVDMIEDAAHETVPIMLVGNKADIRDTAATEGQKCVPGHFGEKLAMTYGALFCETSAKDGSNIVEAVLHLAREVKKRTDKDDSRSITNLTGTNSKKSPQMKNCCNG

SEQ ID NO: 402 - protein, 740 aa, Homo sapiens
MEADGDGEELARLRSVFAACDANRSGRLEREEFRALCTELRVRPADAEAVFQRLDADRDGAITFQEFARGFLGSLRGGRRRDWGPLDPAPAVSEAGPETHDSEEDEGDEDAAAALATSCGPASPGRAWQDFQARLGDEAKFIPREEQVSTLYQNINLVEPRLIQPYEHVIKNFIREIRLQSTEMENLAIAVKRAQDKAAMQLSELEEEMDQRIQAAEHKTRKDEKRKAEEALSDLRRQYETEVGDLQVTIKKLRKLEEQSKRVSQKEDVAALKKQIYDLSMENQKVKKDLLEAQTNIAFLQSELDALKSDYADQSLNTERDLEIIRAYTEDRNSLERQIEILQTANRKLHDSNDGLRSALENSYSKFNRSLHINNISPGNTISRSSPKFIGHSPQPLGYDRSSRSSYVDEDCDSLALCDPLQRTNCEVDSLPESCFDSGLSTLRDPNEYDSEVEYKHQRGFQRSHGVQESFGGDASDTDVPDIRDEETFGLEDVASVLDWKPQGSVSEGSIVSSSRKPISALSPQTDLVDDNAKSFSSQKAYKIVLAGDAAVGKSSFLMRLCKNEFRENISATLGVDFQMKTLIVDGERTVLQLWDTAGQERFRSIAKSYFRKADGVLLLYDVTCEKSFLNIREWVDMIEDAAHETVPIMLVGNKADIRDTAATEGQKCVPGHFGEKLAMTYGALFCETSAKDGSNIVEAVLHLAREVKKRTDKDDSRSITNLTGTNSKKSPQMKNCCNG

SEQ ID NO: 403 - protein, 725 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASQLTQRGALFLLFFLTPAVTPTWYAGSGYYPDESYNEVYAEEVPQAPALDYRVPRWCYTLNIQDGEATCYSPKGGNYHSSLGTRCELSCDRGFRLIGRRSVQCLPSRRWSGTAYCRQMRCHALPFITSGTYTCTNGVLLDSRCDYSCSSGYHLEGDRSRICMEDGRWSGGEPVCVDIDPPKIRCPHSREKMAEPEKLTARVYWDPPLVKDSADGTITRVTLRGPEPGSHFPEGEHVIRYTAYDRAYNRASCKFIVKVQVRRCPTLKPPQHGYLTCTSAGDNYGASCEYHCDGGYDRQGTPSRVCQSSRQWSGSPPICAPMKINVNVNSAAGLLDQFYEKQRLLIISAPDPSNRYYKMQISMLQQSTCGLDLRHVTIIELVGQPPQEVGRIREQQLSANIIEELRQFQRLTRSYFNMVLIDKQGIDRDRYMEPVTPEEIFTFIDDYLLSNQELTQRREQRDICE

SEQ ID NO: 404 - protein, 465 aa, Homo sapiens
MASQLTQRGALFLLFFLTPAVTPTWYAGSGYYPDESYNEVYAEEVPQAPALDYRVPRWCYTLNIQDGEATCYSPKGGNYHSSLGTRCELSCDRGFRLIGRRSVQCLPSRRWSGTAYCRQMRCHALPFITSGTYTCTNGVLLDSRCDYSCSSGYHLEGDRSRICMEDGRWSGGEPVCVDIDPPKIRCPHSREKMAEPEKLTARVYWDPPLVKDSADGTITRVTLRGPEPGSHFPEGEHVIRYTAYDRAYNRASCKFIVKVQVRRCPTLKPPQHGYLTCTSAGDNYGASCEYHCDGGYDRQGTPSRVCQSSRQWSGSPPICAPMKINVNVNSAAGLLDQFYEKQRLLIISAPDPSNRYYKMQISMLQQSTCGLDLRHVTIIELVGQPPQEVGRIREQQLSANIIEELRQFQRLTRSYFNMVLIDKQGIDRDRYMEPVTPEEIFTFIDDYLLSNQELTQRREQRDICE

SEQ ID NO: 405 - protein, 397 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAKGDSTNVDKLVKDIYGGDYERFGLQGSAVASSFGNMMSKEKRDSISKEDLARATLVTITNNIGSIARMCALNENIDRVVFVGNFLRINMVSMKLLAYAMDFWSKGQLKALFLEHEGYFGAVGALLELFKMTDDK

SEQ ID NO: 406 - protein, 137 aa, Homo sapiens
MAAKGDSTNVDKLVKDIYGGDYERFGLQGSAVASSFGNMMSKEKRDSISKEDLARATLVTITNNIGSIARMCALNENIDRVVFVGNFLRINMVSMKLLAYAMDFWSKGQLKALFLEHEGYFGAVGALLELFKMTDDK

SEQ ID NO: 407 - protein, 609 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 408 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 409 - protein, 804 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDDKGDPSNEEAPKAIKPTSKEFRKTWGFRRTTIAKREGAGDAEADPLEPPPPQQQLGLSLRRSGRQPKRTERVEQFLTIARRRGRRSMPVSLEDSGEPTSCPATDAETASEGSVESASETRSGPQSASTAVKERPASSEKVKGGDDHDDTSDSDSDGLTLKELQNRLRRKREQEPTERPLKGIQSRLRKKRREEGPAETVGSEASDTVEGVLPSKQEPENDQGVVSQAGKDDRESKLEGKAAQDIKDEEPGDLGRPKPECEGYDPNALYCICRQPHNNRFMICCDRCEEWFHGDCVGISEARGRLLERNGEDYICPNCTILQVQDETHSETADQQEAKWRPGDADGTDCTSIGTIEQKSSEDQGIKGRIEKAANPSGKKKLKIFQPVIEAPGASKCIGPGCCHVAQPDSVYCSNDCILKHAAATMKFLSSGKEQKPKPKEKMKMKPEKPSLPKCGAQAGIKISSVHKRPAPEKKETTVKKAVVVPARSEALGKEAACESSTPSWASDHNYNAVKPEKTAAPSPSLLYKCSGKYLYSLHPSLIA

SEQ ID NO: 410 - protein, 544 aa, Homo sapiens
MDDKGDPSNEEAPKAIKPTSKEFRKTWGFRRTTIAKREGAGDAEADPLEPPPPQQQLGLSLRRSGRQPKRTERVEQFLTIARRRGRRSMPVSLEDSGEPTSCPATDAETASEGSVESASETRSGPQSASTAVKERPASSEKVKGGDDHDDTSDSDSDGLTLKELQNRLRRKREQEPTERPLKGIQSRLRKKRREEGPAETVGSEASDTVEGVLPSKQEPENDQGVVSQAGKDDRESKLEGKAAQDIKDEEPGDLGRPKPECEGYDPNALYCICRQPHNNRFMICCDRCEEWFHGDCVGISEARGRLLERNGEDYICPNCTILQVQDETHSETADQQEAKWRPGDADGTDCTSIGTIEQKSSEDQGIKGRIEKAANPSGKKKLKIFQPVIEAPGASKCIGPGCCHVAQPDSVYCSNDCILKHAAATMKFLSSGKEQKPKPKEKMKMKPEKPSLPKCGAQAGIKISSVHKRPAPEKKETTVKKAVVVPARSEALGKEAACESSTPSWASDHNYNAVKPEKTAAPSPSLLYKCSGKYLYSLHPSLIA

SEQ ID NO: 411 - protein, 434 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMMGLSNLSPGPGPSQAVPLPEGLLRQRYREEKTLEERRWERLEFLQRKKAFLRHVRRRHRDHMAPYAVGREARISPLGDRSQNRFRCECRYCQSHRPNLSGIPGESNRAPHPSSWETLVQGLSGLTLSLGTNQPGPLPEAALQPQETEEKRQRERQQESKIMFQRLLKQWLEEN

SEQ ID NO: 412 - protein, 174 aa, Homo sapiens
MMGLSNLSPGPGPSQAVPLPEGLLRQRYREEKTLEERRWERLEFLQRKKAFLRHVRRRHRDHMAPYAVGREARISPLGDRSQNRFRCECRYCQSHRPNLSGIPGESNRAPHPSSWETLVQGLSGLTLSLGTNQPGPLPEAALQPQETEEKRQRERQQESKIMFQRLLKQWLEEN

SEQ ID NO: 413 - protein, 574 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 414 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 415 - protein, 574 aa, synthetic construct; identical to SEQ ID NO: 413

SEQ ID NO: 416 - protein, 314 aa, Homo sapiens; identical to SEQ ID NO: 414

SEQ ID NO: 417 - protein, 697 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSPVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPADPGSLRCPVCLSMEGCLEGTTEEICPKGTTHCYDGLLRLRGGGIFSNLRVQGCMPQPGCNLLNGTQEIGPVGMTENCNRKDFLTCHRGTTIMTHGNLAQEPTDWTTSNTEMCEVGQVCQETLLLLDVGLTSTLVGTKGCSTVGAQNSQKTTIHSAPPGVLVASYTHFCSSDLCNSASSSSVLLNSLPPQAAPVPGDRQCPTCVQPLGTCSSGSPRMTCPRGTTHCYDGYIHLSGGGLSTKMSIQGCVAQPSSFLLNHTRQIGIFSAREKRDVQPPASQHEGGGAEGLESLTWGVGLALAPALWWGVVCPSC

SEQ ID NO: 418 - protein, 437 aa, Homo sapiens [sequence omitted]

SEQ ID NO: 419 - protein, 1416 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMFPAAPSPRTPGTGSRRGPLAGLGPGSTPRTASRKGLPLGSAVSSPVLFSPVGRRSSLSSRGTPTRMFPHHSITESVNYDVKTFGSSLPVKVMEALTLAEVDDQLTINIDEGGWACLVCKEKLIIWKIALSPITKLSVCKELQLPPSDFHWSADLVALSYSSPSGEAHSTQAVAVMVATREGSIRYWPSLAGEDTYTEAFVDSGGDKTYSFLTAVQGGSFILSSSGSQLIRLIPESSGKIHQHILPQGQGMLSGIGRKVSSLFGILSPSSDLTLSSVLWDRERSSFYSLTSSNISKWELDDSSEKHAYSWDINRALKENITDAIWGSESNYEAIKEGVNIRYLDFKQNCDGLVILAAAWHSADNPCLIYYSLITIEDNGCQMSDAVTVEVTQYNPPFQSEDLILCQLTVPNFSNQTAYLYNESAVYVCSTGTGKFSLPQEKIVFNAQGDSVLGAGACGGVPIIFSRNSGLVSITSRENVSILAEDLEGSLASSVAGPNSESMIFETTTKNETIAQEDKIKLLKAAFLQYCRKDLGHAQMVVDELFSSHSDLDSDSELDRAVTQISVDLMDDYPASDPRWAESVPEEAPGFSNTSLIILHQLEDKMKAHSFLMDFIHQVGLFGRLGSFPVRGTPMATRLLLCEHAEKLSAAIVLKNHHSRLSDLVNTAILIALNKREYEIPSNLTPADVFFREVSQVDTICECLLEHEEQVLRDAPMDSIEWAEVVINVNNILKDMLQAASHYRQNRNSLYRREESLEKEPEYVPWTATSGPGGIRTVIIRQHEIVLKVAYPQADSNLRNIVTEQLVALIDCFLDGYVSQLKSVDKSSNRERYDNLEMEYLQKRSDLLSPLLSLGQYLWAASLAEKYCDFDILVQMCEQTDNQSRLQRYMTQFADQNFSDFLFRWYLEKGKRGKLLSQPISQHGQLANFLQAHEHLSWLHEINSQELEKAHATLLGLANMETRYFAKKKTLLGLSKLAALASDFSEDMLQEKIEEMAEQERFLLHQETLPEQLLAEKQLNLSAMPVLTAPQLIGLYICEENRRANEYDFKKALDLLEYIDEEEDININDLKLEILCKALQRDNWSSSDGKDDPIEVSKDSIFVKILQKLLKDGIQLSEYLPEVKDLLQADQLGSLKSNPYFEFVLKANYEYYVQGQI

SEQ ID NO: 420 - protein, 1156 aa, Homo sapiens
MFPAAPSPRTPGTGSRRGPLAGLGPGSTPRTASRKGLPLGSAVSSPVLFSPVGRRSSLSSRGTPTRMFPHHSITESVNYDVKTFGSSLPVKVMEALTLAEVDDQLTINIDEGGWACLVCKEKLIIWKIALSPITKLSVCKELQLPPSDFHWSADLVALSYSSPSGEAHSTQAVAVMVATREGSIRYWPSLAGEDTYTEAFVDSGGDKTYSFLTAVQGGSFILSSSGSQLIRLIPESSGKIHQHILPQGQGMLSGIGRKVSSLFGILSPSSDLTLSSVLWDRERSSFYSLTSSNISKWELDDSSEKHAYSWDINRALKENITDAIWGSESNYEAIKEGVNIRYLDFKQNCDGLVILAAAWHSADNPCLIYYSLITIEDNGCQMSDAVTVEVTQYNPPFQSEDLILCQLTVPNFSNQTAYLYNESAVYVCSTGTGKFSLPQEKIVFNAQGDSVLGAGACGGVPIIFSRNSGLVSITSRENVSILAEDLEGSLASSVAGPNSESMIFETTTKNETIAQEDKIKLLKAAFLQYCRKDLGHAQMVVDELFSSHSDLDSDSELDRAVTQISVDLMDDYPASDPRWAESVPEEAPGFSNTSLIILHQLEDKMKAHSFLMDFIHQVGLFGRLGSFPVRGTPMATRLLLCEHAEKLSAAIVLKNHHSRLSDLVNTAILIALNKREYEIPSNLTPADVFFREVSQVDTICECLLEHEEQVLRDAPMDSIEWAEVVINVNNILKDMLQAASHYRQNRNSLYRREESLEKEPEYVPWTATSGPGGIRTVIIRQHEIVLKVAYPQADSNLRNIVTEQLVALIDCFLDGYVSQLKSVDKSSNRERYDNLEMEYLQKRSDLLSPLLSLGQYLWAASLAEKYCDFDILVQMCEQTDNQSRLQRYMTQFADQNFSDFLFRWYLEKGKRGKLLSQPISQHGQLANFLQAHEHLSWLHEINSQELEKAHATLLGLANMETRYFAKKKTLLGLSKLAALASDFSEDMLQEKIEEMAEQERFLLHQETLPEQLLAEKQLNLSAMPVLTAPQLIGLYICEENRRANEYDFKKALDLLEYIDEEEDININDLKLEILCKALQRDNWSSSDGKDDPIEVSKDSIFVKILQKLLKDGIQLSEYLPEVKDLLQADQLGSLKSNPYFEFVLKANYEYYVQGQI

SEQ ID NO: 421 - protein, 737 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMATYTCITCRVAFRDADMQRAHYKTDWHRYNLRRKVASMAPVTAEGFQERVRAQRAVAEEESKGSATYCTVCSKKFASFNAYENHLKSRRHVELEKKAVQAVNRKVEMMNEKNLEKGLGVDSVDKDAMNAAIQQAIKAQPSMSPKKAPPAPAKEARNVVAVGTGGRGTHDRDPSEKPPRLQWFEQQAKKLAKQQEEDSEEEEEDLDGDDWEDIDSDEELECEDTEAMDDVVEQDAEEEEAEEGPPLGAIPITDCLFCSHHSSSLMKNVAHMTKDHSFFIPDIEYLSDIKGLIKYLGEKVGVGKICLWCNEKGKSFYSTEAVQAHMNDKSHCKLFTDGDAALEFADFYDFRSSYPDHKEGEDPNKAEELPSEKNLEYDDETMELILPSGARVGHRSLMRYYKQRFGLSRAVAVAKNRKAVGRVLQQYRALGWTGSTGAALMRERDMQYVQRMKSKWMLKTGMKNNATKQMHFRVQVRF

SEQ ID NO: 422 - protein, 477 aa, Homo sapiens
MATYTCITCRVAFRDADMQRAHYKTDWHRYNLRRKVASMAPVTAEGFQERVRAQRAVAEEESKGSATYCTVCSKKFASFNAYENHLKSRRHVELEKKAVQAVNRKVEMMNEKNLEKGLGVDSVDKDAMNAAIQQAIKAQPSMSPKKAPPAPAKEARNVVAVGTGGRGTHDRDPSEKPPRLQWFEQQAKKLAKQQEEDSEEEEEDLDGDDWEDIDSDEELECEDTEAMDDVVEQDAEEEEAEEGPPLGAIPITDCLFCSHHSSSLMKNVAHMTKDHSFFIPDIEYLSDIKGLIKYLGEKVGVGKICLWCNEKGKSFYSTEAVQAHMNDKSHCKLFTDGDAALEFADFYDFRSSYPDHKEGEDPNKAEELPSEKNLEYDDETMELILPSGARVGHRSLMRYYKQRFGLSRAVAVAKNRKAVGRVLQQYRALGWTGSTGAALMRERDMQYVQRMKSKWMLKTGMKNNATKQMHFRVQVRF

SEQ ID NO: 423 - protein, 649 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEGSEPVAAHQGEEASCSSWGTGSTNKNLPIMSTASVEIDDALYSRQRYVLGDTAMQKMAKSHVFLSGMGGLGLEIAKNLVLAGIKAVTIHDTEKCQAWDLGTNFFLSEDDVVNKRNRAEAVLKHIAELNPYVHVTSSSVPFNETTDLSFLDKYQCVVLTEMKLPLQKKINDFCRSQCPPIKFISADVHGIWSRLFCDFGDEFEVLDTTGEEPKEIFISNITQANPGIVTCLENHPHKLETGQFLTFREINGMTGLNGSIQQITVISPFSFSIGDTTELEPYLHGGIAVQVKTPKTVFFESLERQLKHPKCLIVDFSNPEAPLEIHTAMLALDQFQEKYSRKPNVGCQQDSEELLKLATSISETLEEKVTIEIYGCPNICLLIHKCSVY

SEQ ID NO: 424 - protein, 389 aa, Homo sapiens
MEGSEPVAAHQGEEASCSSWGTGSTNKNLPIMSTASVEIDDALYSRQRYVLGDTAMQKMAKSHVFLSGMGGLGLEIAKNLVLAGIKAVTIHDTEKCQAWDLGTNFFLSEDDVVNKRNRAEAVLKHIAELNPYVHVTSSSVPFNETTDLSFLDKYQCVVLTEMKLPLQKKINDFCRSQCPPIKFISADVHGIWSRLFCDFGDEFEVLDTTGEEPKEIFISNITQANPGIVTCLENHPHKLETGQFLTFREINGMTGLNGSIQQITVISPFSFSIGDTTELEPYLHGGIAVQVKTPKTVFFESLERQLKHPKCLIVDFSNPEAPLEIHTAMLALDQFQEKYSRKPNVGCQQDSEELLKLATSISETLEEKVTIEIYGCPNICLLIHKCSVY

SEQ ID NO: 425 - protein, 465 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLGFLSARQTGLEDPLRLRRAESTRRVLGLELNKDRDVERIHGGGINTLDIEPVEGRYMLSGGSDGVIVLYDLENSSRQSYYTCKAVCSIGRDHPDVHRYSVETVQWYPHDTGMFTSSSFDKTLKVWDTNTLQTADVFNFEETVYSHHMSPVSTKHCLVAVGTRGPKVQLCDLKSGSCSHILQGIFILFQTATTLSKRFNKKKRY

SEQ ID NO: 426 - protein, 205 aa, Homo sapiens
MLGFLSARQTGLEDPLRLRRAESTRRVLGLELNKDRDVERIHGGGINTLDIEPVEGRYMLSGGSDGVIVLYDLENSSRQSYYTCKAVCSIGRDHPDVHRYSVETVQWYPHDTGMFTSSSFDKTLKVWDTNTLQTADVFNFEETVYSHHMSPVSTKHCLVAVGTRGPKVQLCDLKSGSCSHILQGIFILFQTATTLSKRFNKKKRY

SEQ ID NO: 427 - protein, 443 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASNKTTLQKMGKKQNGKSKKVEEAEPEEFVVEKVLDRRVVNGKVEYFLKWKGFTDADNTWEPEENLDCPELIEAFLNSQKAGKEKDGTKRKSLSDSESDDSKSKKKRDAADKPRGFARGLDPERIIGATDSSGELMFLMKWKDSDEADLVLAKEANMKCPQIVIAFYEERLTWHSCPEDEAQ

SEQ ID NO: 428 - protein, 183 aa, Homo sapiens
MASNKTTLQKMGKKQNGKSKKVEEAEPEEFVVEKVLDRRVVNGKVEYFLKWKGFTDADNTWEPEENLDCPELIEAFLNSQKAGKEKDGTKRKSLSDSESDDSKSKKKRDAADKPRGFARGLDPERIIGATDSSGELMFLMKWKDSDEADLVLAKEANMKCPQIVIAFYEERLTWHSCPEDEAQ

SEQ ID NO: 429 - protein, 1043 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVIMSEFSADPAGQGQGQQKPLRVGFYDIERTLGKGNFAVVKLARHRVTKTQVAIKIIDKTRLDSSNLEKIYREVQLMKLLNHPHIIKLYQVMETKDMLYIVTEFAKNGEMFDYLTSNGHLSENEARKKFWQILSAVEYCHDHHIVHRDLKTENLLLDGNMDIKLADFGFGNFYKSGEPLSTWCGSPPYAAPEVFEGKEYEGPQLDIWSLGVVLYVLVCGSLPFDGPNLPTLRQRVLEGRFRIPFFMSQDCESLIRRMLVVDPARRITIAQIRQHRWMRAEPCLPGPACPAFSAHSYTSNLGDYDEQALGIMQTLGVDRQRTVESLQNSSYNHFAAIYYLLLERLKEYRNAQCARPGPARQPRPRSSDLSGLEVPQEGLSTDPFRPALLCPQPQTLVQSVLQAEMDCELQSSLQWPLFFPVDASCSGVFRPRPVSPSSLLDTAISEEARQGPGLEEEQDTQESLPSSTGRRHTLAEVSTRLSPLTAPCIVVSPSTTASPAEGTSSDSCLTFSASKSPAGLSGTPATQGLLGACSPVRLASPFLGSQSATPVLQAQGGLGGAVLLPVSFQEGRRASDTSLTQGLKAFRQQLRKTTRTKGFLGLNKIKGLARQVCQVPASRASRGGLSPFHAPAQSPGLHGGAAGSREGWSLLEEVLEQQRLLQLQHHPAAAPGCSQAPQPAPAPFVIAPCDGPGAAPLPSTLLTSGLPLLPPPLLQTGASPVASAAQLLDTHLHIGTGPTALPAVPPPRLARLAPGCEPLGLLQGDCEMEDLMPCSLGTFVLVQ

SEQ ID NO: 430 - protein, 783 aa, Homo sapiens
MVIMSEFSADPAGQGQGQQKPLRVGFYDIERTLGKGNFAVVKLARHRVTKTQVAIKIIDKTRLDSSNLEKIYREVQLMKLLNHPHIIKLYQVMETKDMLYIVTEFAKNGEMFDYLTSNGHLSENEARKKFWQILSAVEYCHDHHIVHRDLKTENLLLDGNMDIKLADFGFGNFYKSGEPLSTWCGSPPYAAPEVFEGKEYEGPQLDIWSLGVVLYVLVCGSLPFDGPNLPTLRQRVLEGRFRIPFFMSQDCESLIRRMLVVDPARRITIAQIRQHRWMRAEPCLPGPACPAFSAHSYTSNLGDYDEQALGIMQTLGVDRQRTVESLQNSSYNHFAAIYYLLLERLKEYRNAQCARPGPARQPRPRSSDLSGLEVPQEGLSTDPFRPALLCPQPQTLVQSVLQAEMDCELQSSLQWPLFFPVDASCSGVFRPRPVSPSSLLDTAISEEARQGPGLEEEQDTQESLPSSTGRRHTLAEVSTRLSPLTAPCIVVSPSTTASPAEGTSSDSCLTFSASKSPAGLSGTPATQGLLGACSPVRLASPFLGSQSATPVLQAQGGLGGAVLLPVSFQEGRRASDTSLTQGLKAFRQQLRKTTRTKGFLGLNKIKGLARQVCQVPASRASRGGLSPFHAPAQSPGLHGGAAGSREGWSLLEEVLEQQRLLQLQHHPAAAPGCSQAPQPAPAPFVIAPCDGPGAAPLPSTLLTSGLPLLPPPLLQTGASPVASAAQLLDTHLHIGTGPTALPAVPPPRLARLAPGCEPLGLLQGDCEMEDLMPCSLGTFVLVQ

SEQ ID NO: 431 - protein, 850 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 432 - protein, 590 aa, Homo sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 433 - protein, 612 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLRLLASGCARGPGPGVGARPAAGLFHPGRRQSRQASDAPRNQPPSPEFVARPVGVCSMMRLPVQTSPEGLDAAFIGVPLDTGTSNRPGARFGPRRIREESVMLRTVNPSTGALPFQSLMVADLGDVNVNLYNLQDSCRRIQEAYEKIVAAGCIPLTLGGDHTITYPILQAMAKKHGPVGLLHVDAHTDTTDKALGEKLYHGAPFRRCVDEGLLDCKRVVQIGIRGSSTTLDPYRYNRSQGFRVVLAEDCWMKSLVPLMGEVRQQMGGKPIYISFDIDALDPAYAPGTGTPEIAGLTPSQALEIIRGCQGLNVMGCDLVEVSPPYDLSGNTALLAANLLFEMLCALPKVTTV

SEQ ID NO: 434 - protein, 352 aa, Homo sapiens [sequence omitted]

SEQ ID NO: 435 - protein, 697 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGRRMRGAAATAGLWLLALGSLLALWGGLLPPRTELPASRPPEDRLPRRPARSGGPAPAPRFPLPPPLAWDARGGSLKTFRALLTLAAGADGPPRQSRSEPRWHVSARQPRPEESAAVHGGVFWSRGLEEQVPPGFSEAQAAAWLEAARGARMVALERGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCKAKYGRRSGT

SEQ ID NO: 436 - protein, 437 aa, Homo sapiens
MGRRMRGAAATAGLWLLALGSLLALWGGLLPPRTELPASRPPEDRLPRRPARSGGPAPAPRFPLPPPLAWDARGGSLKTFRALLTLAAGADGPPRQSRSEPRWHVSARQPRPEESAAVHGGVFWSRGLEEQVPPGFSEAQAAAWLEAARGARMVALERGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCKAKYGRRSGT

SEQ ID NO: 437 - protein, 726 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMRLRTRKASQQSNQIQTQRTARAKRKYSEVDDSLPSGGEKPSKNETGLLSSIKKFIKGSTPKEERENPSKRSRIERDIDNNLITSTPRAGEKPNKQISRVRRKSQVNGEAGSYEMTNQHVKQNGKLEDNPSSGSPPRTTLLGTIFSPVFNFFSPANKNGTSGSDSPGQAVEAEEIVKQLDMEQVDEITTSTTTSTNGAAYSNQAVQVRPSLNNGLEEAEETVNRDIPPLTAPVTPDSGYSSAHAEATYEEDWEVFDPYYFIKHVPPLTEEQLNRKPALPLKTRSTPEFSLVLDLDETLVHCSLNELEDAALTFPVLFQDVIYQVYVRLRPFFREFLERMSQMYEIILFTASKKVYADKLLNILDPKKQLVRHRLFREHCVCVQGNYIKDLNILGRDLSKTIIIDNSPQAFAYQLSNGIPIESWFMDKNDNELLKLIPFLEKLVELNEDVRPHIRDRFRLHDLLPPD

SEQ ID NO: 438 - protein, 466 aa, Homo sapiens
MRLRTRKASQQSNQIQTQRTARAKRKYSEVDDSLPSGGEKPSKNETGLLSSIKKFIKGSTPKEERENPSKRSRIERDIDNNLITSTPRAGEKPNKQISRVRRKSQVNGEAGSYEMTNQHVKQNGKLEDNPSSGSPPRTTLLGTIFSPVFNFFSPANKNGTSGSDSPGQAVEAEEIVKQLDMEQVDEITTSTTTSTNGAAYSNQAVQVRPSLNNGLEEAEETVNRDIPPLTAPVTPDSGYSSAHAEATYEEDWEVFDPYYFIKHVPPLTEEQLNRKPALPLKTRSTPEFSLVLDLDETLVHCSLNELEDAALTFPVLFQDVIYQVYVRLRPFFREFLERMSQMYEIILFTASKKVYADKLLNILDPKKQLVRHRLFREHCVCVQGNYIKDLNILGRDLSKTIIIDNSPQAFAYQLSNGIPIESWFMDKNDNELLKLIPFLEKLVELNEDVRPHIRDRFRLHDLLPPD

SEQ ID NO: 439 - protein, 359 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVLRRLLAALLHSPQLVERLSESRPIRRAAQLTAFALLQAQLRGQDAARRLQDLAAGPVGSLCRRAERFRDAFTQELRRGLRGRSGPPPGSQRGPGANI

SEQ ID NO: 440 - protein, 99 aa, Homo sapiens
MVLRRLLAALLHSPQLVERLSESRPIRRAAQLTAFALLQAQLRGQDAARRLQDLAAGPVGSLCRRAERFRDAFTQELRRGLRGRSGPPPGSQRGPGANI

SEQ ID NO: 441 - protein, 648 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 442 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 443 - protein, 421 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMCLSSGHIRNEPRPQSWPPPQGWVLRRPPQPLSLDLPAEQTRTSHSPATARSCSWSSTPGWRERRHPGQPASTQALTVEAHVFLGSILKAVLSTTALTRIDLSEPACQYRPAQDWPQGHIGEGGDQALAAAHVGKSMARQEVGPVSLATATVGAGPGAPQP

SEQ ID NO: 444 - protein, 161 aa, Homo sapiens
MCLSSGHIRNEPRPQSWPPPQGWVLRRPPQPLSLDLPAEQTRTSHSPATARSCSWSSTPGWRERRHPGQPASTQALTVEAHVFLGSILKAVLSTTALTRIDLSEPACQYRPAQDWPQGHIGEGGDQALAAAHVGKSMARQEVGPVSLATATVGAGPGAPQP

SEQ ID NO: 445 - protein, 609 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 446 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 447 - protein, 529 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMIRTLLLSTLVAGALSCGVSTYAPDMSRMLGGEEARPNSWPWQVSLQYSSNGQWYHTCGGSLIANSWVLTAAHCISSSGIYRVMLGQHNLYVAESGSLAVSVSKIVVHKDWNSDQVSKGNDIALLKLANPVSLTDKIQLACLPPAGTILPNNYPCYVTGWGRLQTNGALPDDLKQGRLLVVDYATCSSSGWWGSTVKTNMICAGGDGAICTCNGDSGGPLNCQASDGRWEVHGIGSLTSVLGCNYYYKPSIFTRVSNYNDWINSVIANN

SEQ ID NO: 448 - protein, 269 aa, Homo sapiens [sequence omitted]

SEQ ID NO: 449 - protein, 805 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAEASSLGRQSPRVVSCLEHSLCPGEPGLQTTAVVSMGSGDHQFNLAEILSQNYSVRGECEEASRCPDKPKEELEKDFISQSNDMPFDELLALYGYEASDPISDRESEGGDVAPNLPDMTLDKEQIAKDLLSGEEEEETQSSADDLTPSVTSHEASDLFPNRSGSRFLADEDREPGSSASSDTEEDSLPANKCKKEIMVGPQFQADLSNLHLNRHCEKIYENEDQLLWDPSVLPEREVEEFLYRAVKRRWHEMAGPQLPEGEAVKDSEQALYELVKCNFNVEEALRRLRFNVKVIRDGLCAWSEEECRNFEHGFRVHGKNFHLIQANKVRTRSVGECVEYYYLWKKSERYDYFAQQTRLGRRKYVPSGTTDADQDLDGSDPDGPGRPRPEQDTLTGMRTDPLSVDGTAGGLDEPGVASDGLPSSEPGPCSFQQLDESPAVPLSHRPPALADPASYQPAVTAPEPDASPRLAVDFALPKELPLISSHVDLSGDPEETVAPAQVALSVTEFGLIGIGDVNPFLAAHPTCPAPGLHSEPLSHCNVMTC

SEQ ID NO: 450 - protein, 545 aa, Homo sapiens
MAEASSLGRQSPRVVSCLEHSLCPGEPGLQTTAVVSMGSGDHQFNLAEILSQNYSVRGECEEASRCPDKPKEELEKDFISQSNDMPFDELLALYGYEASDPISDRESEGGDVAPNLPDMTLDKEQIAKDLLSGEEEEETQSSADDLTPSVTSHEASDLFPNRSGSRFLADEDREPGSSASSDTEEDSLPANKCKKEIMVGPQFQADLSNLHLNRHCEKIYENEDQLLWDPSVLPEREVEEFLYRAVKRRWHEMAGPQLPEGEAVKDSEQALYELVKCNFNVEEALRRLRFNVKVIRDGLCAWSEEECRNFEHGFRVHGKNFHLIQANKVRTRSVGECVEYYYLWKKSERYDYFAQQTRLGRRKYVPSGTTDADQDLDGSDPDGPGRPRPEQDTLTGMRTDPLSVDGTAGGLDEPGVASDGLPSSEPGPCSFQQLDESPAVPLSHRPPALADPASYQPAVTAPEPDASPRLAVDFALPKELPLISSHVDLSGDPEETVAPAQVALSVTEFGLIGIGDVNPFLAAHPTCPAPGLHSEPLSHCNVMTC

SEQ ID NO: 451 - protein, 805 aa, synthetic construct; identical to SEQ ID NO: 449

SEQ ID NO: 452 - protein, 545 aa, Homo sapiens; identical to SEQ ID NO: 450

SEQ ID NO: 453 - protein, 566 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGCDGGTIPKRHELVKGPKKVEKVDKDAELVAQWNYCTLSQEILRRPIVACELGRLYNKDAVIEFLLDKSAEKALGKAASHIKSIKNVTELKLSDNPAWEGDKGNTKGDKHDDLQRARFICPVVGLEMNGRHRFCFLRCCGCVFSERALKEIKAEVCHTCGAAFQEDDVIVLNGTKEDVDVLKTRMEERRLRAKLEKKTKKPKAAESVSKPDVSEEAPGPSKVKTGKPEEASLDSREKKTNLAPKSTAMNESSSGKAGKPPCGATKRSIADSEESEAYKSLFTTHSSAKRSKEESAHWVTHTSYCF

SEQ ID NO: 454 - protein, 306 aa, Homo sapiens [sequence omitted]

SEQ ID NO: 455 - protein, 424 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSCVLSPILVLHRPLKSKRGDLVFFLCVLWSSSILDRPCMFRKRGSTLCPPKMMAHLSRPGWRLSYQSFCCLGGRLPAADVCPRLSSLQPLSSICRTLGHSCQCFPSVLTFHLSRRLGFHASGPNREMVTQTLRSWCHNPSQQLAGHLSAVACQHAGSSTDGGC

SEQ ID NO: 456 - protein, 164 aa, Homo sapiens
MSCVLSPILVLHRPLKSKRGDLVFFLCVLWSSSILDRPCMFRKRGSTLCPPKMMAHLSRPGWRLSYQSFCCLGGRLPAADVCPRLSSLQPLSSICRTLGHSCQCFPSVLTFHLSRRLGFHASGPNREMVTQTLRSWCHNPSQQLAGHLSAVACQHAGSSTDGGC

SEQ ID NO: 457 - protein, 397 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSMAFLTCCLLKCVKKSKRRRSNRSAQLWSQLKDEDLETVQAAYLGLKHFNKPVSGPSQAHDNHSFTTDHGESTSKLASVTRSVDKDPGIPRALSLSGSSSSPQAQVMVHMANPRQPLPASGLATGMPQQPAAYALG

SEQ ID NO: 458 - protein, 137 aa, Homo sapiens
MSMAFLTCCLLKCVKKSKRRRSNRSAQLWSQLKDEDLETVQAAYLGLKHFNKPVSGPSQAHDNHSFTTDHGESTSKLASVTRSVDKDPGIPRALSLSGSSSSPQAQVMVHMANPRQPLPASGLATGMPQQPAAYALG

SEQ ID NO: 459 - protein, 657 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAQSPPPQSLLGHDHWIFAQGWGWAGHWDSTSPASSSDSSGSCPCDGARGLPQPQPPSCSSRAAEAAATTPRRARTGPAGGQRQSASEREKLRMRTLARALHELRRFLPPSLAPAGQSLTKIETLRLAIRYIGHLSAVLGLSEESLQCRRRQRGDAGSPWGCPLCPDRGPAEAQTQAEGQGQGQGQGQGQGQGQGQGQGQGQGQGRRPGLVSAVLAEASWGSPSACPGAQAAPERLGRGVHDTDPWATPPYCPKIQSPPYSSQGTTSDASLWTPPQGCPWTQSSPEPRNPPVPWTAAPATLELAAVYQGLSVSPEPCLSLGAPSLLPHPSCQRLQPQTPGRCWSHSAEVVPNSEDQGPGAAFQLSEASPPQSSGLRFSGCPELWQEDLEGARLGIFY

SEQ ID NO: 460 - protein, 397 aa, Homo sapiens
MAQSPPPQSLLGHDHWIFAQGWGWAGHWDSTSPASSSDSSGSCPCDGARGLPQPQPPSCSSRAAEAAATTPRRARTGPAGGQRQSASEREKLRMRTLARALHELRRFLPPSLAPAGQSLTKIETLRLAIRYIGHLSAVLGLSEESLQCRRRQRGDAGSPWGCPLCPDRGPAEAQTQAEGQGQGQGQGQGQGQGQGQGQGQGQGQGRRPGLVSAVLAEASWGSPSACPGAQAAPERLGRGVHDTDPWATPPYCPKIQSPPYSSQGTTSDASLWTPPQGCPWTQSSPEPRNPPVPWTAAPATLELAAVYQGLSVSPEPCLSLGAPSLLPHPSCQRLQPQTPGRCWSHSAEVVPNSEDQGPGAAFQLSEASPPQSSGLRFSGCPELWQEDLEGARLGIFY

SEQ ID NO: 461 - protein, 630 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMRRQLRSRRAPSFPYSYRYRLDDPDEANQNYLADEEEEAEEEARVTVVPKSEEEEEEEEKEEEEEEEKEEEEGQGQPTGNAWWQKLQIMSEYLWDPERRMFLARTGQSWSLILLIYFFFYASLAAVITLCMYTLFLTISPYIPTFTERVKPPGVMIRPFAHSLNFNFNVSEPDTWQHYVISLNGFLQGYNDSLQEEMNVDCPPGQYFIQDGNEDEDKKACQFKRSFLKNCSGLEDPTFGYSTGQPCILLKMNRIVGFRPELGDPVKVSCKVQRGDENDIRSISYYPESASFDLRYYPYYGKLTHVNYTSPLVAMHFTDVVKNQAVPVQCQLKGKGVINDVINDRFVGRVIFTLNIETNPAFLYKVVKASS

SEQ ID NO: 462 - protein, 357 aa, Homo sapiens
MRRQLRSRRAPSFPYSYRYRLDDPDEANQNYLADEEEEAEEEARVTVVPKSEEEEEEEEKEEEEEEEKEEEEGQGQPTGNAWWQKLQIMSEYLWDPERRMFLARTGQSWSLILLIYFFFYASLAAVITLCMYTLFLTISPYIPTFTERVKPPGVMIRPFAHSLNFNFNVSEPDTWQHYVISLNGFLQGYNDSLQEEMNVDCPPGQYFIQDGNEDEDKKACQFKRSFLKNCSGLEDPTFGYSTGQPCILLKMNRIVGFRPELGDPVKVSCKVQRGDENDIRSISYYPESASFDLRYYPYYGKLTHVNYTSPLVAMHFTDVVKNQAVPVQCQLKGKGVINDVINDRFVGRVIFTLNIET

SEQ ID NO: 463 - protein, 429 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMFLTEDLITFNLRNFLLFQLWESSFSPGAGGFCTTLPPSFLRVDDRATSSTTDSSRAPSSPRPPGSTSHCGISTRCTERCLCVLPLRTSQVPDVMAPQHDQEKFHDLAYSCLGKSFSMSNQDLYGYSTSSLALGLAWLSWETKKKNVLHLVGLDSLNPAFLYKVVKASS

SEQ ID NO: 464 - protein, 156 aa, Homo sapiens
MFLTEDLITFNLRNFLLFQLWESSFSPGAGGFCTTLPPSFLRVDDRATSSTTDSSRAPSSPRPPGSTSHCGISTRCTERCLCVLPLRTSQVPDVMAPQHDQEKFHDLAYSCLGKSFSMSNQDLYGYSTSSLALGLAWLSWETKKKNVLHLVGLDSL

SEQ ID NO: 465 - protein, 732 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEPSTCRTMESEEDYVEEKESEKCVKEGVTNPSNSSQQALLKADYKALKNGVPSPIMATKIPKKVIAPVDTGDLEAGRRKRRRKRRSLAINLTNCKYESVRRAAQMCGLKEVGEDEEWTLYWTDCAVSLERVMDMKRFQKINHFPGMTEICRKDLLARNLNRMYKLYPSEYNIFPRTWCLPADYGDFQSYGRQRKARTYICKPDSGCQGRGIFITRNPREIKPGEHMICQQYISKPLLIDGFKFDMRVYVLITSCDPLRIFTYEEGLARFATTPYMEPSHNNLDNVCMHLTNYAINKHNENFVRDGAVGSKRKLSTLNIWLQEHSYNPGELWGDIEDIIIKTIISAHSVLRHNYRTCFPQYLNGGTCACFEILGFDILLDHKLKPWLLEVNHSPSFTTDSCLDQEVKDALLCDAMTLVNLRGCDKRKVMEEDKRRVKERLFQCYRQPRESRCARCLACVNPAFLYKVVKASS

SEQ ID NO: 466 - protein, 459 aa, Homo sapiens
MEPSTCRTMESEEDYVEEKESEKCVKEGVTNPSNSSQQALLKADYKALKNGVPSPIMATKIPKKVIAPVDTGDLEAGRRKRRRKRRSLAINLTNCKYESVRRAAQMCGLKEVGEDEEWTLYWTDCAVSLERVMDMKRFQKINHFPGMTEICRKDLLARNLNRMYKLYPSEYNIFPRTWCLPADYGDFQSYGRQRKARTYICKPDSGCQGRGIFITRNPREIKPGEHMICQQYISKPLLIDGFKFDMRVYVLITSCDPLRIFTYEEGLARFATTPYMEPSHNNLDNVCMHLTNYAINKHNENFVRDGAVGSKRKLSTLNIWLQEHSYNPGELWGDIEDIIIKTIISAHSVLRHNYRTCFPQYLNGGTCACFEILGFDILLDHKLKPWLLEVNHSPSFTTDSCLDQEVKDALLCDAMTLVNLRGCDKRKVMEEDKRRVKERLFQCYRQPRESRCARCLACV

SEQ ID NO: 467 - protein, 650 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIEVNGAPACAQKLYAELCQGIVDIAISSVFPPPDVEQPQTQPAAFIKLNPAFLYKVVKASS

SEQ ID NO: 468 - protein, 377 aa, Homo sapiens
MYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIEVNGAPACAQKLYAELCQGIVDIAISSVFPPPDVEQPQTQPAAFIKL

SEQ ID NO: 469 - protein, 753 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEKYENLGLVGEGSYGMVMKCRNKDTGRIVAIKKFLESDDDKMVKKIAMREIKLLKQLRHENLVNLLEVCKKKKRWYLVFEFVDHTILDDLELFPNGLDYQVVQKYLFQIINGIGFCHSHNIIHRDIKPENILVSQSGVVKLCDFGFARTLAAPGEVYTDYVATRWYRAPELLVGDVKYGKAVDVWAIGCLVTEMFMGEPLFPGDSDIDQLYHIMMCLGNLIPRHQELFNKNPVFAGVRLPEIKEREPLERRYPKLSEVVIDLAKKCLHIDPDKRPFCAELLHHDFFQMDGFAERFSQELQLKVQKDARNVSLSKKSQNRKKEKEKDDSLVEERKTLVVQDTNADPKIKDYKLFKIKGSKIDGEKAEKGNRASNASCLHDSRTSHNKIVPSTSLKDCSNVSVDHTRNPSVAIPPLTHNLSAVAPSINSGMGTETIPIQGYRVDEKTKKCSIPFVKPNRHSPSGIYNINVTTLVSGPPLSDDSGADLPQMEHQH

SEQ ID NO: 470 - protein, 493 aa, Homo sapiens
MEKYENLGLVGEGSYGMVMKCRNKDTGRIVAIKKFLESDDDKMVKKIAMREIKLLKQLRHENLVNLLEVCKKKKRWYLVFEFVDHTILDDLELFPNGLDYQVVQKYLFQIINGIGFCHSHNIIHRDIKPENILVSQSGVVKLCDFGFARTLAAPGEVYTDYVATRWYRAPELLVGDVKYGKAVDVWAIGCLVTEMFMGEPLFPGDSDIDQLYHIMMCLGNLIPRHQELFNKNPVFAGVRLPEIKEREPLERRYPKLSEVVIDLAKKCLHIDPDKRPFCAELLHHDFFQMDGFAERFSQELQLKVQKDARNVSLSKKSQNRKKEKEKDDSLVEERKTLVVQDTNADPKIKDYKLFKIKGSKIDGEKAEKGNRASNASCLHDSRTSHNKIVPSTSLKDCSNVSVDHTRNPSVAIPPLTHNLSAVAPSINSGMGTETIPIQGYRVDEKTKKCSIPFVKPNRHSPSGIYNINVTTLVSGPPLSDDSGADLPQMEHQH

SEQ ID NO: 471 - protein, 632 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMESADFYEVEPRPPMSSHLQSPPHAPSNAAFGFPRGAGPAPPPAPPAAPEPLGGICEHETSIDISAYIDPAAFNDEFLADLFQHSRQQEKAKAAAGPAGGGGDFDYPGAPAGPGGAVMSAGAHGPPPGYGCAAAGYLDGRLEPLYERVGAPALRPLVIKQEPREEDEAKQLALAGLFPYQPPPPPPPPHPHASPAHLAAPHLQFQIAHCGQTTMHLQPGHPTPPPTPVPSPHAAPALGAAGLPGPGSALKGLAGAHPDLRTGGGGGGSGAGAGKAKKSVDKNSNEYRVRRERNNIAVRKSRDKAKQRNVETQQKVLELTSDNDRLRKRVEQLSRELDTLRGIFRQLPESSLVKAMGNCANPAFLYKVVKASS

SEQ ID NO: 472 - protein, 359 aa, Homo sapiens
MESADFYEVEPRPPMSSHLQSPPHAPSNAAFGFPRGAGPAPPPAPPAAPEPLGGICEHETSIDISAYIDPAAFNDEFLADLFQHSRQQEKAKAAAGPAGGGGDFDYPGAPAGPGGAVMSAGAHGPPPGYGCAAAGYLDGRLEPLYERVGAPALRPLVIKQEPREEDEAKQLALAGLFPYQPPPPPPPPHPHASPAHLAAPHLQFQIAHCGQTTMHLQPGHPTPPPTPVPSPHAAPALGAAGLPGPGSALKGLAGAHPDLRTGGGGGGSGAGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 473 - protein, 2105 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEDTPPSLSCSDCQRHFPSLPELSRHRELLHPSPNQDSEEADSIPRPYRCQQCGRGYRHPGSLVNHRRTHETGLFPCTTCGKDFSNPMALKSHMRTHAPEGRRRHRPPRPKEATPHLQGETVSTDSWGQRLGSSEGWENQTKHTEETPDCESVPDPRAASGTWEDLPTRQREGLASHPGPEDGADGWGPSTNSARAPPLPIPASSLLSNLEQYLAESVVNFTGGQEPTQSPPAEEERRYKCSQCGKTYKHAGSLTNHRQSHTLGIYPCAICFKEFSNLMALKNHSRLHAQYRPYHCPHCPRVFRLPRELLEHQQSHEGERQEPRWEGKGMPTTNGHTDESSQDQLPSAQMLNGSAELSTSGELEDSGLEEYRPFRCGDCGRTYRHAGSLINHRKSHQTGVYPCSLCSKQLFNAAALKNHVRAHHRPRQGVGENGQPSVPPAPLLLAETTHKEEEDPTTTLDHRPYKCSECGRAYRHRGSLVNHRHSHRTGEYQCSLCPRKYPNLMALRNHVRVHCKAARRSADIGAEGAPSHLKVELPPDPVEAEAAPHTDQDHVCKHEEEATDITPAADKTAAHICSICGLLFEDAESLERHGLTHGAGEKENSRTETTMSPPRAFACRDCGKSYRHSGSLINHRQTHQTGDFSCGACAKHFHTMAAMKNHLRRHSRRRSRRHRKRAGGASGGREAKLLAAESWTRELEDNEGLESPQDPSGESPHGAEGNLESDGDCLQAESEGDKCGLERDETHFQGDKESGGTGEGLERKDASLLDNLDIPGEEGGGTHFCDSLTGVDEDQKPATGQPNSSSHSANAVTGWQAGAAHTCSDCGHSFPHATGLLSHRPCHPPGIYQCSLCPKEFDSLPALRSHFQNHRPGEATSAQPFLCCLCGMIFPGRAGYRLHRRQAHSSSGMTEGSEEEGEEEGVAEAAPARSPPLQLSEAELLNQLQREVEALDSAGYGHICGCCGQTYDDLGSLERHHQSQSSGTTADKAPSPLGVAGDAMEMVVDSVLEDIVNSVSGEGGDAKSQEGAGTPLGDSLCIQGGESLLEAQPRPFRCNQCGKTYRHGGSLVNHRKIHQTGDFLCPVCSRCYPNLAAYRNHLRNHPRCKGSEPQVGPIPEAAGSSELQVGPIPEGGSNKPQHMAEEGPGQAEVEKLQEELKVEPLEEVARVKEEVWEETTVKGEEIEPRLETAEKGCQTEASSERPFSCEVCGRSYKHAGSLINHRQSHQTGHFGCQACSKGFSNLMSLKNHRRIHADPRRFRCSECGKAFRLRKQLASHQRVHMERRGGGGTRKATREDRPFRCGQCGRTYRHAGSLLNHRRSHETGQYSCPTCPKTYSNRMALKDHQRLHSENRRRRAGRSRRTAVRCALCGRSFPGRGSLERHLREHEETEREPANGQGGLDGTAASEANLTGSQGLETQLGGAEPVPHLEDGVPRPGERSQSPIRAASSEAPEPLSWGAGKAGGWPVGGGLGNHSGGWVPQFLTRSEEPEDSVHRSPCHAGDCQLNGPTLSHMDSWDNRDNSSQLQPGSHSSCSQCGKTYCQSGSLLNHNTNKTDRHYCLLCSKEFLNPVATKSHSHNHIDAQTFACPDCGKAFESHQELASHLQAHARGHSQVPAQMEEARDPKAGTGEDQVVLPGQGKAQEAPSETPRGPGESVERARGGQAVTSMAAEDKERPFRCTQCGRSYRHAGSLLNHQKAHTTGLYPCSLCPKLLPNLLSLKNHSRTHTDPKRHCCSICGKAFRTAARLEGHGRVHAPREGPFTCPHCPRHFRRRISFVQHQQQHQEEWTVAGSGAPVAPVTGRGDLPLPPPPTPTTPLLDPSPQWPADLSFSLNPAFLYKVVKASS

SEQ ID NO: 474 - protein, 1832 aa, Homo sapiens
MEDTPPSLSCSDCQRHFPSLPELSRHRELLHPSPNQDSEEADSIPRPYRCQQCGRGYRHPGSLVNHRRTHETGLFPCTTCGKDFSNPMALKSHMRTHAPEGRRRHRPPRPKEATPHLQGETVSTDSWGQRLGSSEGWENQTKHTEETPDCESVPDPRAASGTWEDLPTRQREGLASHPGPEDGADGWGPSTNSARAPPLPIPASSLLSNLEQYLAESVVNFTGGQEPTQSPPAEEERRYKCSQCGKTYKHAGSLTNHRQSHTLGIYPCAICFKEFSNLMALKNHSRLHAQYRPYHCPHCPRVFRLPRELLEHQQSHEGERQEPRWEGKGMPTTNGHTDESSQDQLPSAQMLNGSAELSTSGELEDSGLEEYRPFRCGDCGRTYRHAGSLINHRKSHQTGVYPCSLCSKQLFNAAALKNHVRAHHRPRQGVGENGQPSVPPAPLLLAETTHKEEEDPTTTLDHRPYKCSECGRAYRHRGSLVNHRHSHRTGEYQCSLCPRKYPNLMALRNHVRVHCKAARRSADIGAEGAPSHLKVELPPDPVEAEAAPHTDQDHVCKHEEEATDITPAADKTAAHICSICGLLFEDAESLERHGLTHGAGEKENSRTETTMSPPRAFACRDCGKSYRHSGSLINHRQTHQTGDFSCGACAKHFHTMAAMKNHLRRHSRRRSRRHRKRAGGASGGREAKLLAAESWTRELEDNEGLESPQDPSGESPHGAEGNLESDGDCLQAESEGDKCGLERDETHFQGDKESGGTGEGLERKDASLLDNLDIPGEEGGGTHFCDSLTGVDEDQKPATGQPNSSSHSANAVTGWQAGAAHTCSDCGHSFPHATGLLSHRPCHPPGIYQCSLCPKEFDSLPALRSHFQNHRPGEATSAQPFLCCLCGMIFPGRAGYRLHRRQAHSSSGMTEGSEEEGEEEGVAEAAPARSPPLQLSEAELLNQLQREVEALDSAGYGHICGCCGQTYDDLGSLERHHQSQSSGTTADKAPSPLGVAGDAMEMVVDSVLEDIVNSVSGEGGDAKSQEGAGTPLGDSLCIQGGESLLEAQPRPFRCNQCGKTYRHGGSLVNHRKIHQTGDFLCPVCSRCYPNLAAYRNHLRNHPRCKGSEPQVGPIPEAAGSSELQVGPIPEGGSNKPQHMAEEGPGQAEVEKLQEELKVEPLEEVARVKEEVWEETTVKGEEIEPRLETAEKGCQTEASSERPFSCEVCGRSYKHAGSLINHRQSHQTGHFGCQACSKGFSNLMSLKNHRRIHADPRRFRCSECGKAFRLRKQLASHQRVHMERRGGGGTRKATREDRPFRCGQCGRTYRHAGSLLNHRRSHETGQYSCPTCPKTYSNRMALKDHQRLHSENRRRRAGRSRRTAVRCALCGRSFPGRGSLERHLREHEETEREPANGQGGLDGTAASEANLTGSQGLETQLGGAEPVPHLEDGVPRPGERSQSPIRAASSEAPEPLSWGAGKAGGWPVGGGLGNHSGGWVPQFLTRSEEPEDSVHRSPCHAGDCQLNGPTLSHMDSWDNRDNSSQLQPGSHSSCSQCGKTYCQSGSLLNHNTNKTDRHYCLLCSKEFLNPVATKSHSHNHIDAQTFACPDCGKAFESHQELASHLQAHARGHSQVPAQMEEARDPKAGTGEDQVVLPGQGKAQEAPSETPRGPGESVERARGGQAVTSMAAEDKERPFRCTQCGRSYRHAGSLLNHQKAHTTGLYPCSLCPKLLPNLLSLKNHSRTHTDPKRHCCSICGKAFRTAARLEGHGRVHAPREGPFTCPHCPRHFRRRISFVQHQQQHQEEWTVAGSGAPVAPVTGRGDLPLPPPPTPTTPLLDPSPQWPADLSFSL

SEQ ID NO: 475 - protein, 463 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASPVEASPGCASGLGPHRRKRTTFSVGQLVELERVFAARPYPDISTREHLAQVTHLPEAKIQVWFQNRRAKRIKDRKPGALNSRLELPPNSCSLPDTPQLPWDPGTSSHPLHPTSSAQYTSACPPQTSCLGPILGPGQSWSGAKVAAPWGTSGASGIHSSLEQIVPQTSLGNLSDLIYTSAIVTNVDHFNPAFLYKVVKASS

SEQ ID NO: 476 - protein, 190 aa, Homo sapiens
MASPVEASPGCASGLGPHRRKRTTFSVGQLVELERVFAARPYPDISTREHLAQVTHLPEAKIQVWFQNRRAKRIKDRKPGALNSRLELPPNSCSLPDTPQLPWDPGTSSHPLHPTSSAQYTSACPPQTSCLGPILGPGQSWSGAKVAAPWGTSGASGIHSSLEQIVPQTSLGNLSDLIYTSAIVTNVDHF

SEQ ID NO: 477 - protein, 618 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLPCFQLLRIGGGRGGDLYTFHPPAGAGCTYRLGHRADLCDVALRPQQEPGLISGIHAELHAEPRGDDWRVSLEDHSSQGTLVNNVRLPRGHRLELSDGDLLTFGPEGPPGTSPSEFYFMFQQVRVKPQDFAAITIPRSRGEARVGAGFRPMLPSQGAPQRPLSTFSPAPKATLILNSIGSLSKLRPQPLTFSPSWGGPKSLPVPAPPGEVGTTPSAPPQRNRRKSVHRVLAELDDESEPLENPPPVLMEPRKKLRVDKAPLTPTGNRRGRPRKYPVSAPMAPPAVGGGEPCAAPCCCLPQEETVAWVQCDGCDVWFHVACVGCSIQAAREADFRCPGCRAGIQTNPAFLYKVVKASS

SEQ ID NO: 478 - protein, 345 aa, Homo sapiens [sequence omitted]

SEQ ID NO: 479 - protein, 703 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMWRLVPPKLGRLSRSLKLAALGSLLVLMVLHSPSLLASWQRNELTDRRFLQLNKCPACFGTSWCRRFLNGQVVFEAWGRLRLLDFLNVKNVYFAQYGEPREGGRRRVVLKRLGSQRELAQLDQSICKRATGRPRCDLLQAMPRTEFARLNGDVRLLTPEAVEGWSDLVHCPSQRLLDRLVRRYAETKDSGSFLLRNLKDSERMQLLLTLAFNPEPLVLQSFPSDEGWPFAKYLGACGRMVAVNYVGEELWSYFNAPWEKRVDLAWQLMEIAEQLTNNDFEFALYLLDVSFDNFAVGPRDGKVIIVDAENVLVADKRLIRQNKPENWDVWYESKFDDCDKEACLSFSKEILCARATVDHNYYAVCQNLLSRHATWRGTSGGLLHDPPSEIAKDGRLEALLDECANPKKRYGRFQAAKELREYLAQLSNNVRNPAFLYKVVKASS

SEQ ID NO: 480 - protein, 430 aa, Homo sapiens
MWRLVPPKLGRLSRSLKLAALGSLLVLMVLHSPSLLASWQRNELTDRRFLQLNKCPACFGTSWCRRFLNGQVVFEAWGRLRLLDFLNVKNVYFAQYGEPREGGRRRVVLKRLGSQRELAQLDQSICKRATGRPRCDLLQAMPRTEFARLNGDVRLLTPEAVEGWSDLVHCPSQRLLDRLVRRYAETKDSGSFLLRNLKDSERMQLLLTLAFNPEPLVLQSFPSDEGWPFAKYLGACGRMVAVNYVGEELWSYFNAPWEKRVDLAWQLMEIAEQLTNNDFEFALYLLDVSFDNFAVGPRDGKVIIVDAENVLVADKRLIRQNKPENWDVWYESKFDDCDKEACLSFSKEILCARATVDHNYYAVCQNLLSRHATWRGTSGGLLHDPPSEIAKDGRLEALLDECANPKKRYGRFQAAKELREYLAQLSNNVR

SEQ ID NO: 481 - protein, 989 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDDLTLLDLLECPVCFEKLDVTAKVLPCQHTFCKPCLQRVFKAHKELRCPECRTPVFSNIEALPANLLLVRLLDGVRSGQSSGRGGSFRRPGTMTLQDGRKSRTNPRRLQASPFRLVPNVRIHMDGVPRAKALCNYRGQNPGDLRFNKGDIILLRRQLDENWYQGEINGISGNFPASSVEVIKQLPQPPPLCRALYNFDLRGKDKSENQDCLTFLKDDIITVISRVDENWAEGKLGDKVGIFPILFVEPNLTARHLLEKNKGRQSSCTKNLSLVSSSSRGNTSTLRRGPGSRRKVPGQFSITTALNTLNRMVHSPSGRHMVEISTPVLISSSNPSVITQPMEKADVPSSCVGQVSTYHPAPVSPGHSTAVVSLPGSQQHLSANMFVALHSYSAHGPDELDLQKGEGVRVLGKCQDGWLRGVSLVTGRVGIFPNNYVIPIFRKTSSFPDSRSPGLYTTWTLSTSSVSSQGSISEGDPRQSRPFKSVFVPTAIVNPVRSTAGPGTLGQGSLRKGRSSMRKNGSLQRPLQSGIPTLVVGSLRRSPTMVLRPQQFQFYQPQGIPSSPSAVVVEMGSKPALTGEPALTCISRGSEARIHSAASSLIMEDKEIPIKSEPLPKPPASAPPSILVKPENSRNGIEKQVKTVRFQNYSPPPTKHYTSHPTSGKPEQPATLKASQPEAASLGPEMTVLFAHRSGCHSGQQTDLRRKSALAKATTLVSTASGTQTVFPSK

SEQ ID NO: 482 - protein, 729 aa, Homo sapiens
MDDLTLLDLLECPVCFEKLDVTAKVLPCQHTFCKPCLQRVFKAHKELRCPECRTPVFSNIEALPANLLLVRLLDGVRSGQSSGRGGSFRRPGTMTLQDGRKSRTNPRRLQASPFRLVPNVRIHMDGVPRAKALCNYRGQNPGDLRFNKGDIILLRRQLDENWYQGEINGISGNFPASSVEVIKQLPQPPPLCRALYNFDLRGKDKSENQDCLTFLKDDIITVISRVDENWAEGKLGDKVGIFPILFVEPNLTARHLLEKNKGRQSSCTKNLSLVSSSSRGNTSTLRRGPGSRRKVPGQFSITTALNTLNRMVHSPSGRHMVEISTPVLISSSNPSVITQPMEKADVPSSCVGQVSTYHPAPVSPGHSTAVVSLPGSQQHLSANMFVALHSYSAHGPDELDLQKGEGVRVLGKCQDGWLRGVSLVTGRVGIFPNNYVIPIFRKTSSFPDSRSPGLYTTWTLSTSSVSSQGSISEGDPRQSRPFKSVFVPTAIVNPVRSTAGPGTLGQGSLRKGRSSMRKNGSLQRPLQSGIPTLVVGSLRRSPTMVLRPQQFQFYQPQGIPSSPSAVVVEMGSKPALTGEPALTCISRGSEARIHSAASSLIMEDKEIPIKSEPLPKPPASAPPSILVKPENSRNGIEKQVKTVRFQNYSPPPTKHYTSHPTSGKPEQPATLKASQPEAASLGPEMTVLFAHRSGCHSGQQTDLRRKSALAKATTLVSTASGTQTVFPSK

SEQ ID NO: 483 - protein, 439 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMELQGAQEDLGISLSSPRRNHETRPGSKAKGRSSICLQASVWMAGGKLRLRASEHLTQGHQQELRDWNLGEDASLLFSKSPFGAGKLIQAPAHVFRQCWVQGNAWISCITKFDSKRSPEVASSPSYLTVPRRSPLPVFLRPSDRCVCGGCYLGKSTRRRACQSLLSDPLGVTFPTQTRP

SEQ ID NO: 484 - protein, 179 aa, Homo sapiens
MELQGAQEDLGISLSSPRRNHETRPGSKAKGRSSICLQASVWMAGGKLRLRASEHLTQGHQQELRDWNLGEDASLLFSKSPFGAGKLIQAPAHVFRQCWVQGNAWISCITKFDSKRSPEVASSPSYLTVPRRSPLPVFLRPSDRCVCGGCYLGKSTRRRACQSLLSDPLGVTFPTQTRP

SEQ ID NO: 485 - protein, 337 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGLLTFRDVAIEFSLEEWQCLDTAQKNLYRNVMLENYRNLAFLGIAVSKPDLIICLEKEKEPWNMKRDEMVDEPPGR

SEQ ID NO: 486 - protein, 77 aa, Homo sapiens
MGLLTFRDVAIEFSLEEWQCLDTAQKNLYRNVMLENYRNLAFLGIAVSKPDLIICLEKEKEPWNMKRDEMVDEPPGR

SEQ ID NO: 487 - protein, 380 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAETSALPTGFGELEVLAVGMVLLVEDPGAADSLPPTGAELGSCGQWDQPECPRCSHIQPSPCLPQALALRLGRLPGSRLPGLCDSVGQHLQQCSHRMGALSPLLHPPKCCHRSHHFEWR

SEQ ID NO: 488 - protein, 120 aa, Homo sapiens
MAETSALPTGFGELEVLAVGMVLLVEDPGAADSLPPTGAELGSCGQWDQPECPRCSHIQPSPCLPQALALRLGRLPGSRLPGLCDSVGQHLQQCSHRMGALSPLLHPPKCCHRSHHFEWR

SEQ ID NO: 489 - protein, 573 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDNKKRLAYAIIQFLHDQLRHGGLSSDAQESLEVAIQCLETAFGVTVEDSDLALPQTLPEIFEAAATGKEMPQDLRSPARTPPSEEDSAEAERLKTEGNEQMKVENFEAAVHFYGKAIELNPANAVYFCNRAAAYSKLGNYAGAVQDCERAICIDPAYSKAYGRMGLALSSLNKHVEAVAYYKKALELDPDNETYKSNLKIAELKLREAPSPTGGVGSFDIAGLLNNPGFMSMASNLMNNPQIQQLMSGMISGGNNPLGTPGTSPSQNDLASLIQAGQQFAQQMQQQNPELIEQLRSQIRSRTPSASNDDQQE

SEQ ID NO: 490 - protein, 313 aa, Homo sapiens
MDNKKRLAYAIIQFLHDQLRHGGLSSDAQESLEVAIQCLETAFGVTVEDSDLALPQTLPEIFEAAATGKEMPQDLRSPARTPPSEEDSAEAERLKTEGNEQMKVENFEAAVHFYGKAIELNPANAVYFCNRAAAYSKLGNYAGAVQDCERAICIDPAYSKAYGRMGLALSSLNKHVEAVAYYKKALELDPDNETYKSNLKIAELKLREAPSPTGGVGSFDIAGLLNNPGFMSMASNLMNNPQIQQLMSGMISGGNNPLGTPGTSPSQNDLASLIQAGQQFAQQMQQQNPELIEQLRSQIRSRTPSASNDDQQE

SEQ ID NO: 491 - protein, 695 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNGIIESLILPGIISIHPVVRNLAVLCLGCCGLQNQDFARKHFVLLLQVLQIDDVTIKISALKAIFDQLMTFGIEPFKTKKIKTLHCEGTEINSDDEQESKEVEETATAKNVLKLLSDFLDSEVSELRTGAAEGLAKLMFSGLLVSSRILSRLILLWYNPVTEEDVQLRHCLGVFFPVFAYASRTNQECFEEAFLPTLQTLANAPASSPLAEIDITNVAELLVDLTRPSGLNPQAKTSQDYQALTVHDNLAMKICNEILTSPCSPEIRVYTKALSSLELSSHLAKDLLVLLNEILEQVKDRTCLRALEKIKIQLEKGNKEFGDQAEAAQDATLTTTTFQNEDEKNKEVYMTPLRGVKATQASKSTQLKTNRGQRKVTVSARTNRRCQTAEADSESDHEVPEPESEMKMRLPRRAKTAALEKSKLNLAQFLNEDLS

SEQ ID NO: 492 - protein, 435 aa, Homo sapiens
MNGIIESLILPGIISIHPVVRNLAVLCLGCCGLQNQDFARKHFVLLLQVLQIDDVTIKISALKAIFDQLMTFGIEPFKTKKIKTLHCEGTEINSDDEQESKEVEETATAKNVLKLLSDFLDSEVSELRTGAAEGLAKLMFSGLLVSSRILSRLILLWYNPVTEEDVQLRHCLGVFFPVFAYASRTNQECFEEAFLPTLQTLANAPASSPLAEIDITNVAELLVDLTRPSGLNPQAKTSQDYQALTVHDNLAMKICNEILTSPCSPEIRVYTKALSSLELSSHLAKDLLVLLNEILEQVKDRTCLRALEKIKIQLEKGNKEFGDQAEAAQDATLTTTTFQNEDEKNKEVYMTPLRGVKATQASKSTQLKTNRGQRKVTVSARTNRRCQTAEADSESDHEVPEPESEMKMRLPRRAKTAALEKSKLNLAQFLNEDLS

SEQ ID NO: 493 - protein, 417 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPKNAVVILRYGPYSAAGLPVEHHTFRLQGLQAVLAIDGHEVILEKIEDWNVVELMVNEEVIFHCNIKDLEFGKLTPSSDKRTTSSSRLTFHQLSSPCGMKVSPLQQFPQKTQDLTCIVLAQIGSCIHFQTNLCDLGWPGLDHMLISGLEKRGTQPY

SEQ ID NO: 494 - protein, 157 aa, Homo sapiens
MPKNAVVILRYGPYSAAGLPVEHHTFRLQGLQAVLAIDGHEVILEKIEDWNVVELMVNEEVIFHCNIKDLEFGKLTPSSDKRTTSSSRLTFHQLSSPCGMKVSPLQQFPQKTQDLTCIVLAQIGSCIHFQTNLCDLGWPGLDHMLISGLEKRGTQPY

SEQ ID NO: 495 - protein, 789 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSSTVSYWILNSTRNSIATLQGGRRLYSRYVSNRNKLKWRLFSRVPPTLNSSPCGGFTLCKAYRHTSTEEDDFHLQLSPEQINEVLRAGETTHKILDLESRVPNSVLRFESNQLAANSPVEDRRGVASCLQTNGLMFGIFDGHGGHACAQAVSERLFYYVAVSLMSHQTLEHMEGAMESMKPLLPILHWLKHPGDSIYKDVTSVHLDHLRVYWQELLDLHMEMGLSIEEALMYSFQRLDSDISLEIQAPLEDEVTRNLSLQVAFSGATACMAHVDGIHLHVANAGDCRAILGVQEDNGMWSCLPLTRDHNAWNQAELSRLKREHPESEDRTIIMEDRLLGVLIPCRAFGDVQLKWSKELQRSILERGFNTEALNIYQFTPPHYYTPPYLTAEPEVTYHRLRPQDKFLVLASDGLWDMLSNEDVVRLVVGHLAEADWHKTDLAQRPANLGLMQSLLLQRKASGLHEADQNAATRLIRHAIGNNEYGEMEAERLAAMLTLPEDLARMYRDDITVTVVYFNSESIGAYYKGG

SEQ ID NO: 496 - protein, 529 aa, Homo sapiens
MSSTVSYWILNSTRNSIATLQGGRRLYSRYVSNRNKLKWRLFSRVPPTLNSSPCGGFTLCKAYRHTSTEEDDFHLQLSPEQINEVLRAGETTHKILDLESRVPNSVLRFESNQLAANSPVEDRRGVASCLQTNGLMFGIFDGHGGHACAQAVSERLFYYVAVSLMSHQTLEHMEGAMESMKPLLPILHWLKHPGDSIYKDVTSVHLDHLRVYWQELLDLHMEMGLSIEEALMYSFQRLDSDISLEIQAPLEDEVTRNLSLQVAFSGATACMAHVDGIHLHVANAGDCRAILGVQEDNGMWSCLPLTRDHNAWNQAELSRLKREHPESEDRTIIMEDRLLGVLIPCRAFGDVQLKWSKELQRSILERGFNTEALNIYQFTPPHYYTPPYLTAEPEVTYHRLRPQDKFLVLASDGLWDMLSNEDVVRLVVGHLAEADWHKTDLAQRPANLGLMQSLLLQRKASGLHEADQNAATRLIRHAIGNNEYGEMEAERLAAMLTLPEDLARMYRDDITVTVVYFNSESIGAYYKGG

SEQ ID NO: 497 - protein, 751 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVLLWGLLVLSWSCLQGPCSVFSPVSAMEPLGRQLTSGPNQEQVSPLTLLKLGNQEPGGQTALKSPPGVCSRDPTPEQTHRLARAMMAFTADLFSLVAQTSTCPNLILSPLSVALALSHLALGAQNHTLQRLQQVLHAGSGPCLPHLLSRLCQDLGPGAFRLAARMYLQKGFPIKEDFLEQSEQLFGAKPVSLTGKQEDDLANINQWVKEATEGKIQEFLSGLPEDTVLLLLNAIHFQGFWRNKFDPSLTQRDSFHLDEQFTVPVEMMQARTYPLRWFLLEQPEIQVAHFPFKNNMSFVVLVPTHFEWNVSQVLANLSWDTLHPPLVWERPTKVRLPKLYLKHQMDLVATLSQLGLQELFQAPDLRGISEQSLVVSGVQHQSTLELSEVGVEAAAATSIAMSRMSLSSFSVNRPFLFFIFEDTTGLPLFVGSVRNPNPSAPRELKEQQDSPGNKDFLQSLKGFPRGDKLFGPDLKLVPPMEEDYPQFGSPK

SEQ ID NO: 498 - protein, 491 aa, Homo sapiens
MVLLWGLLVLSWSCLQGPCSVFSPVSAMEPLGRQLTSGPNQEQVSPLTLLKLGNQEPGGQTALKSPPGVCSRDPTPEQTHRLARAMMAFTADLFSLVAQTSTCPNLILSPLSVALALSHLALGAQNHTLQRLQQVLHAGSGPCLPHLLSRLCQDLGPGAFRLAARMYLQKGFPIKEDFLEQSEQLFGAKPVSLTGKQEDDLANINQWVKEATEGKIQEFLSGLPEDTVLLLLNAIHFQGFWRNKFDPSLTQRDSFHLDEQFTVPVEMMQARTYPLRWFLLEQPEIQVAHFPFKNNMSFVVLVPTHFEWNVSQVLANLSWDTLHPPLVWERPTKVRLPKLYLKHQMDLVATLSQLGLQELFQAPDLRGISEQSLVVSGVQHQSTLELSEVGVEAAAATSIAMSRMSLSSFSVNRPFLFFIFEDTTGLPLFVGSVRNPNPSAPRELKEQQDSPGNKDFLQSLKGFPRGDKLFGPDLKLVPPMEEDYPQFGSPK

SEQ ID NO: 499 - protein, 737 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSSSRKDHLGASSSEPLPVIIVGNGPSGICLSYLLSGYTPYTKPDAIHPHPLLQRKLTEAPGVSILDQDLDYLSEGLEGRSQSPVALLFDALLRPDTDFGGNMKSVLTWKHRKEHAIPHVVLGRNLPGGAWHSIEGSMVILSQGQWMGLPDLEVKDWMQKKRRGLRNSRATAGDIAHYYRDYVVKKGLGHNFVSGAVVTAVEWGTPDPSSCGAQDSSPLFQVSGFLTRNQAQQPFSLWARNVVLATGTFDSPARLGIPGEALPFIHHELSALEAATRVGAVTPASDPVLIIGAGLSAADAVLYARHYNIPVIHAFRRAVDDPGLVFNQLPKMLYPEYHKVHQMMREQSILSPSPYEGYRSLPRHQLLCFKEDCQAVFQDLEGVEKVFGVSLVLVLIGSHPDLSFLPGAGADFAVDPDQPLSAKRNPIDVDPFTYQSTRQEGLYAMGPLAGDNFVRFVQGGALAVASSLLRKETRKPP

SEQ ID NO: 500 - protein, 477 aa, Homo sapiens
MSSSRKDHLGASSSEPLPVIIVGNGPSGICLSYLLSGYTPYTKPDAIHPHPLLQRKLTEAPGVSILDQDLDYLSEGLEGRSQSPVALLFDALLRPDTDFGGNMKSVLTWKHRKEHAIPHVVLGRNLPGGAWHSIEGSMVILSQGQWMGLPDLEVKDWMQKKRRGLRNSRATAGDIAHYYRDYVVKKGLGHNFVSGAVVTAVEWGTPDPSSCGAQDSSPLFQVSGFLTRNQAQQPFSLWARNVVLATGTFDSPARLGIPGEALPFIHHELSALEAATRVGAVTPASDPVLIIGAGLSAADAVLYARHYNIPVIHAFRRAVDDPGLVFNQLPKMLYPEYHKVHQMMREQSILSPSPYEGYRSLPRHQLLCFKEDCQAVFQDLEGVEKVFGVSLVLVLIGSHPDLSFLPGAGADFAVDPDQPLSAKRNPIDVDPFTYQSTRQEGLYAMGPLAGDNFVRFVQGGALAVASSLLRKETRKPP

SEQ ID NO: 501 - protein, 691 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAEITNIRPSFDVSPVVAGLIGASVLVVCVSVTVFVWSCCHQQAEKKHKNPPYKFIHMLKGISIYPETLSNKKKIIKVRRDKDGPGREGGRRNLLVDAAEAGLLSRDKDPRGPSSGSCIDQLPIKMDYGEELRSPITSLTPGESKTTSPSSPEEDVMLGSLTFSVDYNFPKKALVVTIQEAHGLPVMDDQTQGSDPYIKMTILPDKRHRVKTRVLRKTLDPVFDETFTFYGIPYSQLQDLVLHFLVLSFDRFSRDDVIGEVMVPLAGVDPSTGKVQLTRDIIKRNIQKCISRGELQVSLSYQPVAQRMTVVVLKARHLPKMDITGLSGNPYVKVNVYYGRKRIAKKKTHVKKCTLNPIFNESFIYDIPTDLLPDISIEFLVIDFDRTTKNEVVGRLILGAHSVTASGAEHWREVCESPRKPVAKWHSLSEY

SEQ ID NO: 502 - protein, 431 aa, Homo sapiens
MAEITNIRPSFDVSPVVAGLIGASVLVVCVSVTVFVWSCCHQQAEKKHKNPPYKFIHMLKGISIYPETLSNKKKIIKVRRDKDGPGREGGRRNLLVDAAEAGLLSRDKDPRGPSSGSCIDQLPIKMDYGEELRSPITSLTPGESKTTSPSSPEEDVMLGSLTFSVDYNFPKKALVVTIQEAHGLPVMDDQTQGSDPYIKMTILPDKRHRVKTRVLRKTLDPVFDETFTFYGIPYSQLQDLVLHFLVLSFDRFSRDDVIGEVMVPLAGVDPSTGKVQLTRDIIKRNIQKCISRGELQVSLSYQPVAQRMTVVVLKARHLPKMDITGLSGNPYVKVNVYYGRKRIAKKKTHVKKCTLNPIFNESFIYDIPTDLLPDISIEFLVIDFDRTTKNEVVGRLILGAHSVTASGAEHWREVCESPRKPVAKWHSLSEY

SEQ ID NO: 503 - protein, 520 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMMDPCSVGVQLRTTNECHKTYYTRHTGFKTLQELSSNDMLLLQLRTGMTLSGNNTICFHHVKIYIDRFEDLQKSCCDPFNIHKKLAKKNLHVIDLDDATFLSAKFGRQLVPGWKLCPKCTQIINGSVDVDTEDRQKRKPESDGRTAKALRSLQFTNPGRQTEFAPETGKREKRRLTKNATAGSDRQVIPAKSKVYDSQGLLIFSGMDLCDCLDEDCLGCFYACPACGSTKCGAECRCDRKWLYEQIEIEGGEIIHNKHAG

SEQ ID NO: 504 - protein, 260 aa, Homo sapiens
MMDPCSVGVQLRTTNECHKTYYTRHTGFKTLQELSSNDMLLLQLRTGMTLSGNNTICFHHVKIYIDRFEDLQKSCCDPFNIHKKLAKKNLHVIDLDDATFLSAKFGRQLVPGWKLCPKCTQIINGSVDVDTEDRQKRKPESDGRTAKALRSLQFTNPGRQTEFAPETGKREKRRLTKNATAGSDRQVIPAKSKVYDSQGLLIFSGMDLCDCLDEDCLGCFYACPACGSTKCGAECRCDRKWLYEQIEIEGGEIIHNKHAG

SEQ ID NO: 505 - protein, 796 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 506 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 507 - protein, 1629 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGGKNKKHKAPAAAVVRAAVSASRAKSAEAGIAGEAQSKKPVSRPATAAAAAAGSREPRVKQGPKIYSFNSTNDSSGPANLDKSILKVVINNKLEQRIIGVINEHKKQNNDKGMISGRLTAKKLQDLYMALQAFSFKTKDIEDAMTNTLLYGGDLHSALDWLCLNLSDDALPEGFSQEFEEQQPKSRPKFQSPQIQATISPPLQPKTKTYEEDPKSKPKKEEKNMEVNMKEWILRYAEQQNEEEKNENSKSLEEEEKFDPNERYLHLAAKLLDAKEQAATFKLEKNKQGQKEAQEKIRKFQREMETLEDHPVFNPAMKISHQQNERKKPPVATEGESALNFNLFEKSAAATEEEKDKKKEPHDVRNFDYTARSWTGKSPKQFLIDWVRKNLPKSPNPSFEKVPVGRYWKCRVRVIKSEDDVLVVCPTILTEDGMQAQHLGATLALYRLVKGQSVHQLLPPTYRDVWLEWSDAEKKREELNKMETNKPRDLFIAKLLNKLKQQQQQQQQHSENKRENSEDPEESWENLVSDEDFSALSLESANVEDLEPVRNLFRKLQSTPKYQKLLKERQQLPVFKHRDSIVETLKRHRVVVVAGETGSGKSTQVPHFLLEDLLLNEWEASKCNIVCTQHRRISAVSLANRVCDELGCENGPGGRNSLCGYQIRMESRACESTRLLYCTTGVLLRKLQEDGLLSNVSHVIVDEVHERSVQSDFLLIILKEILQKRSDLHLILMSATVDSEKFSTYFTHCPILRISGRSYPVEVFHLEDIIEETGFVLEKDSEYCQKFLEEEEEVTINVTSKAGGIKKYQEYIPVQTGAHADLNPFYQKYSSRTQHAILYMNPHKINLDLILELLAYLDKSPQFRNIEGAVLIFLPGLAHIQQLYDLLSNDRRFYSERYKVIALHSILSTQDQAAAFTLPPPGVRKIVLATNIAETGITIPDVVFVIDTGRTKENKYHESSQMSSLVETFVSKASALQRQGRAGRVRDGFCFRMYTRERFEGFMDYSVPEILRVPLEELCLHIMKCNLGSPEDFLSKALDPPQLQVISNAMNLLRKIGACELNEPKLTPLGQHLAALPVNVKIGKMLIFGAIFGCLDPVATLAAVMTEKSPFTTPIGRKDEADLAKSALAMADSDHLTIYNAYLGWKKARQEGGYRSEITYCRRNFLNRTSLLTLEDVKQELIKLVKAAGFSSSTTSTSWEGNRASQTLSFQEIALLKAVLVAGLYDNVGKIIYTKSVDVTEKLACIVETAQGKAQVHPSSVNRDLQTHGWLLYQEKIRYARVYLRETTLITPFPVLLFGGDIEVQHRERLLSIDGWIYFQAPVKIAVIFKQLRVLIDSVLRKKLENPKMSLENDKILQIITELIKTENN

SEQ ID NO: 508 - protein, 1369 aa, Homo sapiens
MGGKNKKHKAPAAAVVRAAVSASRAKSAEAGIAGEAQSKKPVSRPATAAAAAAGSREPRVKQGPKIYSFNSTNDSSGPANLDKSILKVVINNKLEQRIIGVINEHKKQNNDKGMISGRLTAKKLQDLYMALQAFSFKTKDIEDAMTNTLLYGGDLHSALDWLCLNLSDDALPEGFSQEFEEQQPKSRPKFQSPQIQATISPPLQPKTKTYEEDPKSKPKKEEKNMEVNMKEWILRYAEQQNEEEKNENSKSLEEEEKFDPNERYLHLAAKLLDAKEQAATFKLEKNKQGQKEAQEKIRKFQREMETLEDHPVFNPAMKISHQQNERKKPPVATEGESALNFNLFEKSAAATEEEKDKKKEPHDVRNFDYTARSWTGKSPKQFLIDWVRKNLPKSPNPSFEKVPVGRYWKCRVRVIKSEDDVLVVCPTILTEDGMQAQHLGATLALYRLVKGQSVHQLLPPTYRDVWLEWSDAEKKREELNKMETNKPRDLFIAKLLNKLKQQQQQQQQHSENKRENSEDPEESWENLVSDEDFSALSLESANVEDLEPVRNLFRKLQSTPKYQKLLKERQQLPVFKHRDSIVETLKRHRVVVVAGETGSGKSTQVPHFLLEDLLLNEWEASKCNIVCTQHRRISAVSLANRVCDELGCENGPGGRNSLCGYQIRMESRACESTRLLYCTTGVLLRKLQEDGLLSNVSHVIVDEVHERSVQSDFLLIILKEILQKRSDLHLILMSATVDSEKFSTYFTHCPILRISGRSYPVEVFHLEDIIEETGFVLEKDSEYCQKFLEEEEEVTINVTSKAGGIKKYQEYIPVQTGAHADLNPFYQKYSSRTQHAILYMNPHKINLDLILELLAYLDKSPQFRNIEGAVLIFLPGLAHIQQLYDLLSNDRRFYSERYKVIALHSILSTQDQAAAFTLPPPGVRKIVLATNIAETGITIPDVVFVIDTGRTKENKYHESSQMSSLVETFVSKASALQRQGRAGRVRDGFCFRMYTRERFEGFMDYSVPEILRVPLEELCLHIMKCNLGSPEDFLSKALDPPQLQVISNAMNLLRKIGACELNEPKLTPLGQHLAALPVNVKIGKMLIFGAIFGCLDPVATLAAVMTEKSPFTTPIGRKDEADLAKSALAMADSDHLTIYNAYLGWKKARQEGGYRSEITYCRRNFLNRTSLLTLEDVKQELIKLVKAAGFSSSTTSTSWEGNRASQTLSFQEIALLKAVLVAGLYDNVGKIIYTKSVDVTEKLACIVETAQGKAQVHPSSVNRDLQTHGWLLYQEKIRYARVYLRETTLITPFPVLLFGGDIEVQHRERLLSIDGWIYFQAPVKIAVIFKQLRVLIDSVLRKKLENPKMSLENDKILQIITELIKTENN

SEQ ID NO: 509 - protein, 1073 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDATALERDAVQFARLAVQRDHEGRYSEAVFYYKEAAQALIYAEMAGSSLENIQEKITEYLERVQALHSAVQSKSADPLKSKHQLDLERAHFLVTQAFDEDEKENVEDAIELYTEAVDLCLKTSYETADKVLQNKLKQLARQALDRAEALSEPLTKPVGKISSTSVKPKPPPVRAHFPLGANPFLERPQSFISPQSCDAQGQRYTAEEIEVLRTTSKINGIEYVPFMNVDLRERFAYPMPFCDRWGKLPLSPKQKTTFSKWVRPEDLTNNPTMIYTVSSFSIKQTIVSDCSFVASLAISAAYERRFNKKLITGIIYPQNKDGEPEYNPCGKYMVKLHLNGVPRKVIIDDQLPVDHKGELLCSYSNNKSELWVSLIEKAYMKVMGGYDFPGSNSNIDLHALTGWIPERIAMHSDSQTFSKDNSFRMLYQRFHKGDVLITASTGMMTEAEGEKWGLVPTHAYAVLDIREFKGLRFIQLKNPWSHLRWKGRYSENDVKNWTPELQKYLNFDPRTAQKIDNGIFWISWDDLCQYYDVIYLSWNPGLFKESTCIHSTWDAKQGPVKDAYSLANNPQYKLEVQCPQGGAAVWVLLSRHITDKDDFANNREFITMVVYKTDGKKVYYPADPPPYIDGIRINSPHYLTKIKLTTPGTHTFTLVVSQYEKQNTIHYTVRVYSACSFTFSKIPSPYTLSKRINGKWSGQSAGGCGNFQETHKNNPIYQFHIEKTGPLLIELRGPRQYSVGFEVVTVSTLGDPGPHGFLRKSSGDYRCGFCYLELENIPSGIFNIIPSTFLPKQEGPFFLDFNSIIPIKITQLQ

SEQ ID NO: 510 - protein, 813 aa, Homo sapiens
MDATALERDAVQFARLAVQRDHEGRYSEAVFYYKEAAQALIYAEMAGSSLENIQEKITEYLERVQALHSAVQSKSADPLKSKHQLDLERAHFLVTQAFDEDEKENVEDAIELYTEAVDLCLKTSYETADKVLQNKLKQLARQALDRAEALSEPLTKPVGKISSTSVKPKPPPVRAHFPLGANPFLERPQSFISPQSCDAQGQRYTAEEIEVLRTTSKINGIEYVPFMNVDLRERFAYPMPFCDRWGKLPLSPKQKTTFSKWVRPEDLTNNPTMIYTVSSFSIKQTIVSDCSFVASLAISAAYERRFNKKLITGIIYPQNKDGEPEYNPCGKYMVKLHLNGVPRKVIIDDQLPVDHKGELLCSYSNNKSELWVSLIEKAYMKVMGGYDFPGSNSNIDLHALTGWIPERIAMHSDSQTFSKDNSFRMLYQRFHKGDVLITASTGMMTEAEGEKWGLVPTHAYAVLDIREFKGLRFIQLKNPWSHLRWKGRYSENDVKNWTPELQKYLNFDPRTAQKIDNGIFWISWDDLCQYYDVIYLSWNPGLFKESTCIHSTWDAKQGPVKDAYSLANNPQYKLEVQCPQGGAAVWVLLSRHITDKDDFANNREFITMVVYKTDGKKVYYPADPPPYIDGIRINSPHYLTKIKLTTPGTHTFTLVVSQYEKQNTIHYTVRVYSACSFTFSKIPSPYTLSKRINGKWSGQSAGGCGNFQETHKNNPIYQFHIEKTGPLLIELRGPRQYSVGFEVVTVSTLGDPGPHGFLRKSSGDYRCGFCYLELENIPSGIFNIIPSTFLPKQEGPFFLDFNSIIPIKITQLQ

SEQ ID NO: 511 - protein, 354 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 512 - protein, 94 aa, Homo sapiens
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 513 - protein, 775 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGHNGSWISPNASEPHNASGAEAAGVNRSALGEFGEAQLYRQFTTTVQVVIFIGSLLGNFMVLWSTCRTTVFKSVTNRFIKNLACSGICASLVCVPFDIILSTSPHCCWWIYTMLFCKVVKFLHKVFCSVTILSFPAIALDRYYSVLYPLERKISDAKSRELVMYIWAHAVVASVPVFAVTNVADIYATSTCTEVWSNSLGHLVYVLVYNITTVIVPVVVVFLFLILIRRALSASQKKKVIIAALRTPQNTISIPYASQREAELHATLLSMVMVFILCSVPYATLVVYQTVLNVPDTSVFLLLTAVWLPKVSLLANPVLFLTVNKSVRKCLIGTLVQLHHRYSRRNVVSTGSGMAEASLEPSIRSGSQLLEMFHIGQQQIFKPTEDEEESEAKYIGSADFQAKEIFSTCLEGEQGPQFAPSAPPLSTVDSVSQVAPAAPVEPETFPDKYSLQFGFGPFELPPQWLSETRNSKKRLLPPLGNTPEELIQTKVPKVGRVERKMSRNNKVSIFPKVDS

SEQ ID NO: 514 - protein, 515 aa, Homo sapiens
MGHNGSWISPNASEPHNASGAEAAGVNRSALGEFGEAQLYRQFTTTVQVVIFIGSLLGNFMVLWSTCRTTVFKSVTNRFIKNLACSGICASLVCVPFDIILSTSPHCCWWIYTMLFCKVVKFLHKVFCSVTILSFPAIALDRYYSVLYPLERKISDAKSRELVMYIWAHAVVASVPVFAVTNVADIYATSTCTEVWSNSLGHLVYVLVYNITTVIVPVVVVFLFLILIRRALSASQKKKVIIAALRTPQNTISIPYASQREAELHATLLSMVMVFILCSVPYATLVVYQTVLNVPDTSVFLLLTAVWLPKVSLLANPVLFLTVNKSVRKCLIGTLVQLHHRYSRRNVVSTGSGMAEASLEPSIRSGSQLLEMFHIGQQQIFKPTEDEEESEAKYIGSADFQAKEIFSTCLEGEQGPQFAPSAPPLSTVDSVSQVAPAAPVEPETFPDKYSLQFGFGPFELPPQWLSETRNSKKRLLPPLGNTPEELIQTKVPKVGRVERKMSRNNKVSIFPKVDS

SEQ ID NO: 515 - protein, 911 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 516 - protein, 651 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 517 - protein, 851 aa, synthetic construct; identical to SEQ ID NO: 261

SEQ ID NO: 518 - protein, 591 aa, Homo sapiens; identical to SEQ ID NO: 262

SEQ ID NO: 519 - protein, 660 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMENKAMYLHTVSDCDTSSICEDSFDGRSLSKLNLCEDGPCHKRRASICCTQLGSLSALKHAVLGLYLLVFLILVGIFILAVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASKDILLGPWDMVLAQG

SEQ ID NO: 520 - protein, 400 aa, Homo sapiens
MENKAMYLHTVSDCDTSSICEDSFDGRSLSKLNLCEDGPCHKRRASICCTQLGSLSALKHAVLGLYLLVFLILVGIFILAVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASKDILLGPWDMVLAQG

SEQ ID NO: 521 - protein, 681 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMDLENKVKKMGLGHEQGFGAPCLKCKEKCEGFELHFWRKICRNCKCGQEEHDVLLSNEEDRKVGKLFEDTKYTTLIAKLKSDGIPMYKRNVMILTNPVAAKKNVSINTVTYEWAPPVQNQALARQYMQMLPKEKQPVAGSEGAQYRKKQLAKQLPAHDQDPSKCHELSPREVKEMEQFVKKYKSEALGVGDVKLPCEMDAQGPKQMNIPGGDRSTPAAVGAMEDKSAEHKRTQYSCYCCKLSMKEGDPAIYAERAGYDKLWHPACFVCSTCHELLVDMIYFWKNEKLYCGRHYCDSEKPRCAGCDELIFSNEYTQAENQNWHLKHFCCFDCDSILAGEIYVMVNDKPVCKPCYVKNHAVVCQGCHNAIDPEVQRVTYNNFSWHASTECFLCSCCSKCLIGQKFMPVEGMVFCSVECKKRMS

SEQ ID NO: 522 - protein, 421 aa, Homo sapiens
MDLENKVKKMGLGHEQGFGAPCLKCKEKCEGFELHFWRKICRNCKCGQEEHDVLLSNEEDRKVGKLFEDTKYTTLIAKLKSDGIPMYKRNVMILTNPVAAKKNVSINTVTYEWAPPVQNQALARQYMQMLPKEKQPVAGSEGAQYRKKQLAKQLPAHDQDPSKCHELSPREVKEMEQFVKKYKSEALGVGDVKLPCEMDAQGPKQMNIPGGDRSTPAAVGAMEDKSAEHKRTQYSCYCCKLSMKEGDPAIYAERAGYDKLWHPACFVCSTCHELLVDMIYFWKNEKLYCGRHYCDSEKPRCAGCDELIFSNEYTQAENQNWHLKHFCCFDCDSILAGEIYVMVNDKPVCKPCYVKNHAVVCQGCHNAIDPEVQRVTYNNFSWHASTECFLCSCCSKCLIGQKFMPVEGMVFCSVECKKRMS

SEQ ID NO: 523 - protein, 731 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAAMETETAPLTLESLPTDPLLLILSFLDYRDLINCCYVSRRLSQLSSHDPLWRRHCKKYWLISEEEKTQKNQCWKSLFIDTYSDVGRYIDHYAAIKKAWDDLKKYLEPRCPRMVLSLKEGAREEDLDAVEAQIGCKLPDDYRCSYRIHNGQKLVVPGLLGSMALSNHYRSEDLLDVDTAAGGFQQRQGLKYCLPLTFCIHTGLSQYIAVEAAEGRNKNEVFYQCPDQMARNPAAIDMFIIGATFTDWFTSYVKNVVSGGFPIIRDQIFRYVHDPECVATTGDITVSVSTSFLPELSSVHPPHYFFTYRIRIEMSKDALPEKACQLDSRYWRITNAKGDVEEVQGPGVVGEFPIISPGRVYEYTSCTTFSTTSGYMEGYYTFHFLYFKDKIFNVAIPRFHMACPTFRVSIARLEMGPDEYEEMEEEEEEEEEEDEDDDSADMDESDEDDEEERRRRVFDVPIRRRRCSRLF

SEQ ID NO: 524 - protein, 471 aa, Homo sapiens
MAAMETETAPLTLESLPTDPLLLILSFLDYRDLINCCYVSRRLSQLSSHDPLWRRHCKKYWLISEEEKTQKNQCWKSLFIDTYSDVGRYIDHYAAIKKAWDDLKKYLEPRCPRMVLSLKEGAREEDLDAVEAQIGCKLPDDYRCSYRIHNGQKLVVPGLLGSMALSNHYRSEDLLDVDTAAGGFQQRQGLKYCLPLTFCIHTGLSQYIAVEAAEGRNKNEVFYQCPDQMARNPAAIDMFIIGATFTDWFTSYVKNVVSGGFPIIRDQIFRYVHDPECVATTGDITVSVSTSFLPELSSVHPPHYFFTYRIRIEMSKDALPEKACQLDSRYWRITNAKGDVEEVQGPGVVGEFPIISPGRVYEYTSCTTFSTTSGYMEGYYTFHFLYFKDKIFNVAIPRFHMACPTFRVSIARLEMGPDEYEEMEEEEEEEEEEDEDDDSADMDESDEDDEEERRRRVFDVPIRRRRCSRLF

SEQ ID NO: 525 - protein, 588 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMTGSNEFKLNQPPEDGISSVKFSPNTSQFLLVSSWDTSVRLYDVPANSMRLKYQHTGAVLDCAFYDPTHAWSGGLDHQLKMHDLNTDQENLVGTHDAPIRCVEYCPEVNVMVTGSWDQTVKLWDPRTPCNAGTFSQPEKVYTLSVSGDRLIVGTAGRRVLVWDLRNMGYVQQRRESSLKYQTRCIRAFPNKQGYVLSSIEGRVAVEYLDPSPEVQKKKYAFKCHRLKENNIEQIYPVNAISFHNIHNTFATGGSDGFVNIWDPFNKKRLCQFHRYPTSIASLAFSNDGTTLAIASSYMYEMDDTEHPEDGIFIRQVTDAETKPKSPCT

SEQ ID NO: 526 - protein, 328 aa, Homo sapiens
MTGSNEFKLNQPPEDGISSVKFSPNTSQFLLVSSWDTSVRLYDVPANSMRLKYQHTGAVLDCAFYDPTHAWSGGLDHQLKMHDLNTDQENLVGTHDAPIRCVEYCPEVNVMVTGSWDQTVKLWDPRTPCNAGTFSQPEKVYTLSVSGDRLIVGTAGRRVLVWDLRNMGYVQQRRESSLKYQTRCIRAFPNKQGYVLSSIEGRVAVEYLDPSPEVQKKKYAFKCHRLKENNIEQIYPVNAISFHNIHNTFATGGSDGFVNIWDPFNKKRLCQFHRYPTSIASLAFSNDGTTLAIASSYMYEMDDTEHPEDGIFIRQVTDAETKPKSPCT

SEQ ID NO: 527 - protein, 507 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNPFLILAFVGAAVAVPFDDDDKIVGGYTCEENSLPYQVSLNSGSHFCGGSLISEQWVVSAAHCYKTRIQVRLGEHNIKVLEGNEQFINAAKIIRHPKYNRDTLDNDIMLIKLSSPAVINARVSTISLPTAPPAAGTECLISGWGNTLSFGADYPDELKCLDAPVLTQAECKASYPGKITNSMFCVGFLEGGKDSCQRDSGGPVVCNGQLQGVVSWGHGCAWKNRPGVYTKVYNYVDWIKDTIAANS

SEQ ID NO: 528 - protein, 247 aa, Homo sapiens
MNPFLILAFVGAAVAVPFDDDDKIVGGYTCEENSLPYQVSLNSGSHFCGGSLISEQWVVSAAHCYKTRIQVRLGEHNIKVLEGNEQFINAAKIIRHPKYNRDTLDNDIMLIKLSSPAVINARVSTISLPTAPPAAGTECLISGWGNTLSFGADYPDELKCLDAPVLTQAECKASYPGKITNSMFCVGFLEGGKDSCQRDSGGPVVCNGQLQGVVSWGHGCAWKNRPGVYTKVYNYVDWIKDTIAANS

SEQ ID NO: 529 - protein, 423 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMVGGGGVGGGLLENANPLIYQRSGERPVTAGEEDEQVPDSIDAREIFDLIRSINDPEHPLTLEELNVVEQVRVQVSDPESTVAVAFTPTIPHCSMATLIGLSIKVKLLRSLPQRFKMDVHITPGTHASEHAVNKQLADKERVAAALENTHLLEVVNQCLSARS

SEQ ID NO: 530 - protein, 163 aa, Homo sapiens
MVGGGGVGGGLLENANPLIYQRSGERPVTAGEEDEQVPDSIDAREIFDLIRSINDPEHPLTLEELNVVEQVRVQVSDPESTVAVAFTPTIPHCSMATLIGLSIKVKLLRSLPQRFKMDVHITPGTHASEHAVNKQLADKERVAAALENTHLLEVVNQCLSARS

SEQ ID NO: 531 - protein, 488 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMELGLGGLSTLSHCPWPRQQAPLGLSAQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 532 - protein, 228 aa, Homo sapiens
MELGLGGLSTLSHCPWPRQQAPLGLSAQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 533 - protein, 714 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRRGQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 534 - protein, 454 aa, Homo sapiens
MARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRRGQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 535 - protein, 743 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASLGDLVRAWHLGAQAVDRGDWARALHLFSGVPAPPARLCFNAGCVHLLAGDPEAALRAFDQAVTKDTCMAVGFFQRGVANFQLARFQEALSDFWLALEQLRGHAAIDYTQLGLRFKLQAWEVLHNVASAQCQLGLWTEAASSLREAMSKWPEGSLNGLDSALDQVQRRGSLPPRQVPRGEVFRPHRWHLKHLEPVDFLGKAKVVASAIPDDQGWGVRPQQPQGPGANHDARSLIMDSPRAGTHQGPLDAETEVGADRCTSTAYQEQRPQVEQVGKQAPLSPGLPAMGGPGPGPCEDPAGAGGAGAGGSEPLVTVTVQCAFTVALRARRGADLSSLRALLGQALPHQAQLGQLSYLAPGEDGHWVPIPEEESLQRAWQDAAACPRGLQLQCRGAGGRPVLYQVVAQHSYSAQGPEDLGFRQGDTVDVLCEEPDVPLAVDQAWLEGHCDGRIGIFPKCFVVPAGPRMSGAPGRLPRSQQGDQP

SEQ ID NO: 536 - protein, 483 aa, Homo sapiens
MASLGDLVRAWHLGAQAVDRGDWARALHLFSGVPAPPARLCFNAGCVHLLAGDPEAALRAFDQAVTKDTCMAVGFFQRGVANFQLARFQEALSDFWLALEQLRGHAAIDYTQLGLRFKLQAWEVLHNVASAQCQLGLWTEAASSLREAMSKWPEGSLNGLDSALDQVQRRGSLPPRQVPRGEVFRPHRWHLKHLEPVDFLGKAKVVASAIPDDQGWGVRPQQPQGPGANHDARSLIMDSPRAGTHQGPLDAETEVGADRCTSTAYQEQRPQVEQVGKQAPLSPGLPAMGGPGPGPCEDPAGAGGAGAGGSEPLVTVTVQCAFTVALRARRGADLSSLRALLGQALPHQAQLGQLSYLAPGEDGHWVPIPEEESLQRAWQDAAACPRGLQLQCRGAGGRPVLYQVVAQHSYSAQGPEDLGFRQGDTVDVLCEEPDVPLAVDQAWLEGHCDGRIGIFPKCFVVPAGPRMSGAPGRLPRSQQGDQP

SEQ ID NO: 537 - protein, 601 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMATWRRDGRLTGGQRLLCAGLAGTLSLSLTAPLELATVLAQVGVVRGHARGPWATGHRVWRAEGLRALWKGNAVACLRLFPCSAVQLAAYRKFVVLFTDDLGHISQWSSIMAGSLAGMVSTIVTYPTDLIKTRLIMQNILEPSYRGLLHAFSTIYQQEGFLALYRGVSLTVVGALPFSAGSLLVYMNLEKIWNGPRDQFSLPQNFANVCLAAAVTQTLSFPFETVKRKMQAQSPYLPHSGGVDVHFSGAVDCFRQIVKAQGVLGLWNGLTANLLKIVPYFGIMFSTFEFCKRICLYQNGYILSPLSYKLTPGVDQSLQPQELRELKKFFKTRKLKPKKPTL

SEQ ID NO: 538 - protein, 341 aa, Homo sapiens
MATWRRDGRLTGGQRLLCAGLAGTLSLSLTAPLELATVLAQVGVVRGHARGPWATGHRVWRAEGLRALWKGNAVACLRLFPCSAVQLAAYRKFVVLFTDDLGHISQWSSIMAGSLAGMVSTIVTYPTDLIKTRLIMQNILEPSYRGLLHAFSTIYQQEGFLALYRGVSLTVVGALPFSAGSLLVYMNLEKIWNGPRDQFSLPQNFANVCLAAAVTQTLSFPFETVKRKMQAQSPYLPHSGGVDVHFSGAVDCFRQIVKAQGVLGLWNGLTANLLKIVPYFGIMFSTFEFCKRICLYQNGYILSPLSYKLTPGVDQSLQPQELRELKKFFKTRKLKPKKPTL

SEQ ID NO: 539 - protein, 637 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMFNPHALDSPAVIFDNGSGFCKAGLSGEFGPRHMVSSIVGHLKFQAPSAEANQKKYFVGEEALYKQEALQLHSPFERGLITGWDDVERLWKHLFEWELGVKPSDQPLLATEPSLNPRENREKMAEVMFENFGVPAFYLSDQAVLALYASACVTGLVVDSGDAVTCTVPIFEGYSLPHAVTKLHVAGRDITELLMQLLLASGHTFPCQLDKGLVDDIKKKLCYVALEPEKELSRRPEEVLREYKLPDGNIISLGDPLHQAPEALFVPQQLGSQSPGLSNMVSSSITKCDTDIQKILFGEIVLSGGTTLFHGLDDRLLKELEQLASKDTPIKITAPPDRWFSTWIGASIVTSLSSFKQMWVTAADFKEFGTSVVQRRCF

SEQ ID NO: 540 - protein, 377 aa, Homo sapiens
MFNPHALDSPAVIFDNGSGFCKAGLSGEFGPRHMVSSIVGHLKFQAPSAEANQKKYFVGEEALYKQEALQLHSPFERGLITGWDDVERLWKHLFEWELGVKPSDQPLLATEPSLNPRENREKMAEVMFENFGVPAFYLSDQAVLALYASACVTGLVVDSGDAVTCTVPIFEGYSLPHAVTKLHVAGRDITELLMQLLLASGHTFPCQLDKGLVDDIKKKLCYVALEPEKELSRRPEEVLREYKLPDGNIISLGDPLHQAPEALFVPQQLGSQSPGLSNMVSSSITKCDTDIQKILFGEIVLSGGTTLFHGLDDRLLKELEQLASKDTPIKITAPPDRWFSTWIGASIVTSLSSFKQMWVTAADFKEFGTSVVQRRCF

SEQ ID NO: 541 - protein, 773 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 542 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 543 - protein, 875 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMPASRLRDRAASSASGSTCGSMSQTHPVLESGLLASAGCSAPRGPRKGGPAPVDRKAKASAMPDSPAEVKTQPRSTPPSMPPPPPAASQGATRPPSFTPHTLMNGSSHSPTAINGAPCTPNGFSNGPATSSTASLSTQHLPPACGARQLSKLKRFLTTLQQFGSDISPEIGERVRTLVLGLVNSTLTIEEFHSKLQEATNFPLRPFVIPFLKANLPLLQRELLHCARLAKQTPAQYLAQHEQLLLDASASSPIDSSELLLEVNENGKRRTPDRTKENGSDRDPLHPQRYSPSNGPPQPTPPPHYRLEDIAMAHHFRDAYRHPDPRELRERHRPLVVPGSRQEEVIDHKLTEREWAEEWKHLNNLLNCIMDMVEKTRRSLTVLRRCQEADREELNHWARRYSDAEDTKKGPAPAAARPRSSSAGPEGPQLDVPREFLPRTLTGYVPEDIWRKAEEAVNEVKRQAMSELQKAVSDAERKAHELITTERAKMERALAEAKRQASEDALTVINQQEDSSESCWNCGRKASETCSGCNAARYCGSFCQHRDWEKHHHVCGQSLQGPTAVVADPVPGPPEAAHSLGPSLPVGAASPSEAGSAGPSRPGSPSPPGPLDTVPR

SEQ ID NO: 544 - protein, 615 aa, Homo sapiens
MPASRLRDRAASSASGSTCGSMSQTHPVLESGLLASAGCSAPRGPRKGGPAPVDRKAKASAMPDSPAEVKTQPRSTPPSMPPPPPAASQGATRPPSFTPHTLMNGSSHSPTAINGAPCTPNGFSNGPATSSTASLSTQHLPPACGARQLSKLKRFLTTLQQFGSDISPEIGERVRTLVLGLVNSTLTIEEFHSKLQEATNFPLRPFVIPFLKANLPLLQRELLHCARLAKQTPAQYLAQHEQLLLDASASSPIDSSELLLEVNENGKRRTPDRTKENGSDRDPLHPQRYSPSNGPPQPTPPPHYRLEDIAMAHHFRDAYRHPDPRELRERHRPLVVPGSRQEEVIDHKLTEREWAEEWKHLNNLLNCIMDMVEKTRRSLTVLRRCQEADREELNHWARRYSDAEDTKKGPAPAAARPRSSSAGPEGPQLDVPREFLPRTLTGYVPEDIWRKAEEAVNEVKRQAMSELQKAVSDAERKAHELITTERAKMERALAEAKRQASEDALTVINQQEDSSESCWNCGRKASETCSGCNAARYCGSFCQHRDWEKHHHVCGQSLQGPTAVVADPVPGPPEAAHSLGPSLPVGAASPSEAGSAGPSRPGSPSPPGPLDTVPR

SEQ ID NO: 545 - protein, 433 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSLLGPKVLLFLAAFIITSDWIPLGVNSQRGDDVTQATPETFTEDPNLVNDPATDETVLAVLADIAPSTDDLASLSEKNTTAECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 546 - protein, 173 aa, Homo sapiens
MSLLGPKVLLFLAAFIITSDWIPLGVNSQRGDDVTQATPETFTEDPNLVNDPATDETVLAVLADIAPSTDDLASLSEKNTTAECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 547 - protein, 1229 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMASKRKSTTPCMIPVKTVVLQDASMEAQPAETLPEGPQQDLPPEASAASSEAAQNPSSTDGSTLANGHRSTLDGYLYSCKYCDFRSHDMTQFVGHMNSEHTDFNKDPTFVCSGCSFLAKTPEGLSLHNATCHSGEASFVWNVAKPDNHVVVEQSIPESTSTPDLAGEPSAEGADGQAEIIITKTPIMKIMKGKAEAKKIHTLKENVPSQPVGEALPKLSTGEMEVREGDHSFINGAVPVSQASASSAKNPHAANGPLIGTVPVLPAGIAQFLSLQQQPPVHAQHHVHQPLPTAKALPKVMIPLSSIPTYNAAMDSNSFLKNSFHKFPYPTKAELCYLTVVTKYPEEQLKIWFTAQRLKQGISWSPEEIEDARKKMFNTVIQSVPQPTITVLNTPLVASAGNVQHLIQAALPGHVVGQPEGTGGGLLVTQPLMANGLQATSSPLPLTVTSVPKQPGVAPINTVCSNTTSAVKVVNAAQSLLTACPSITSQAFLDASIYKNKKSHEQLSALKGSFCRNQFPGQSEVEHLTKVTGLSTREVRKWFSDRRYHCRNLKGSRAMIPGDHSSIIIDSVPEVSFSPSSKVPEVTCIPTTATLATHPSAKRQSWHQTPDFTPTKYKERAPEQLRALESSFAQNPLPLDEEMDRLRSETKMTRREIDSWFSERRKKVNAEETKKAEENASQEEEEAAEDEGGEEDLASELRVSGENGSLEMPSSHILAERKVSPIKINLKNLRVTEANGRNEIPGLGACDPEDDESNKLAEQLPGKVSCKKTAQQRHLLRQLFVQTQWPSNQDYDSIMAQTGLPRPEVVRWFGDSRYALKNGQLKWYEDYKRGNFPPGLLVIAPGNRELLQDYYMTHKMLYEEDLQNLCDKTQMSSQQKQTEFDLINVKDWPVWETACHVEEPNPTLCCHMPFPCPAAGHLGELPESSQTAQSLPLPSACPPPSKQQARWGSHQFFLPQCRTFPLPSNG

SEQ ID NO: 548 - protein, 969 aa, Homo sapiens [sequence omitted]

SEQ ID NO: 549 - protein, 458 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMIEETSFGKSYAQLNFENSMLWMDLPDEEPDRLSNKLLQYDPSQDQWSVRAPMKYSKYRFSTAVVNSEIYVLGGIGCVGQDKGQVRKCLDVVEIYNPDGDFWREGPPMPSPLLSLRTNSTNAGAVDGKLYVCGGFHGADRHEVISKEILELDPWENQWNVVAINVLMHDSYDVCLVARMNPRDLIPPPSDLVEEGNEH

SEQ ID NO: 550 - protein, 198 aa, Homo sapiens
MIEETSFGKSYAQLNFENSMLWMDLPDEEPDRLSNKLLQYDPSQDQWSVRAPMKYSKYRFSTAVVNSEIYVLGGIGCVGQDKGQVRKCLDVVEIYNPDGDFWREGPPMPSPLLSLRTNSTNAGAVDGKLYVCGGFHGADRHEVISKEILELDPWENQWNVVAINVLMHDSYDVCLVARMNPRDLIPPPSDLVEEGNEH

SEQ ID NO: 551 - protein, 629 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMGFPAERLEGVYRNNIDDVVRFLDSKHKNHYKIHNLCAERHYDTAKSNYRVAQYPFEDHNPPQLELIKPFCEDLDQWLSEDDNHVAAIHCKAGKGRTGIMIYAYLLHRGKFLKAQEALDFYGEVRTRDKKGVTIPSQRRYVYYYSYLVKNHVDYRPVALLFHKMMFETIPMFSGGTCNPQFVVCQLKVKMYSSNSGPTRWEDKFMYFEFPQPLPVCGGIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV

SEQ ID NO: 552 - protein, 369 aa, Homo sapiens
MGFPAERLEGVYRNNIDDVVRFLDSKHKNHYKIHNLCAERHYDTAKSNYRVAQYPFEDHNPPQLELIKPFCEDLDQWLSEDDNHVAAIHCKAGKGRTGIMIYAYLLHRGKFLKAQEALDFYGEVRTRDKKGVTIPSQRRYVYYYSYLVKNHVDYRPVALLFHKMMFETIPMFSGGTCNPQFVVCQLKVKMYSSNSGPTRWEDKFMYFEFPQPLPVCGGIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV

SEQ ID NO: 553 - protein, 457 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMISKMLKMQMLEDEDDLAYAETEKKTRTDSTSDGRPAWMRTLHTTASNWLHLIPQTLSHLKRTVENIKDPLFRFFEREVKMGAKLLQDVRQDLADVVQVCEGKKKQTNYLRTLINELVKGILPRSWSHYTVPAGMTVIQWVSDFSERIKQLQNISLAAASGGAKELKVKALLTSLGWSAAVLGWGGSGSGEKHRAQV

SEQ ID NO: 554 - protein, 197 aa, Homo sapiens
MISKMLKMQMLEDEDDLAYAETEKKTRTDSTSDGRPAWMRTLHTTASNWLHLIPQTLSHLKRTVENIKDPLFRFFEREVKMGAKLLQDVRQDLADVVQVCEGKKKQTNYLRTLINELVKGILPRSWSHYTVPAGMTVIQWVSDFSERIKQLQNISLAAASGGAKELKVKALLTSLGWSAAVLGWGGSGSGEKHRAQV

SEQ ID NO: 555 - protein, 752 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMSLQAPRRLLELAGQSLLGDQALAISILDELPRELFPPLFVEAFTSRRCEVLKVMVQAWPFPCLPLGSLMKTPDLEILHYVVDGIDCLLAQKVRPRRRKLQVLELRDVDENFWTIWSGARPLSCSPEAMSKRQTVEDCPRTGEKQPLKVFMDVCLKEKFMDEDLSFFSGWVQHRRGSVHLCCTKVVNYSMSILNFRNILETVYPDSIQVLEIWNMCWPCMIVEFIRYLSQMRNLRKLFISDGCRYLLSSDSQEQLVAEFSSVLLRLEYLQMLYVRRVCFFRGHLDQLIRCLRSPLETLALTYGFLEKVDLKCLPRYPSLSQLKQLNLSHGALRFIRLEPLRALLEKVAATLQTLFLVDCGIRDSKLRVILPALSCCSNLTTFCFHGNDTSMDGLKDLLRHTGRLSNLSLETYPAPRESLDDRGRVISELLTPLQAELMRILREVREPKRIFFGPVSCPCCGTSPTEQLEFNFCLWGRPANPAFLYKVVKASS

SEQ ID NO: 556 - protein, 479 aa, Homo sapiens
MSLQAPRRLLELAGQSLLGDQALAISILDELPRELFPPLFVEAFTSRRCEVLKVMVQAWPFPCLPLGSLMKTPDLEILHYVVDGIDCLLAQKVRPRRRKLQVLELRDVDENFWTIWSGARPLSCSPEAMSKRQTVEDCPRTGEKQPLKVFMDVCLKEKFMDEDLSFFSGWVQHRRGSVHLCCTKVVNYSMSILNFRNILETVYPDSIQVLEIWNMCWPCMIVEFIRYLSQMRNLRKLFISDGCRYLLSSDSQEQLVAEFSSVLLRLEYLQMLYVRRVCFFRGHLDQLIRCLRSPLETLALTYGFLEKVDLKCLPRYPSLSQLKQLNLSHGALRFIRLEPLRALLEKVAATLQTLFLVDCGIRDSKLRVILPALSCCSNLTTFCFHGNDTSMDGLKDLLRHTGRLSNLSLETYPAPRESLDDRGRVISELLTPLQAELMRILREVREPKRIFFGPVSCPCCGTSPTEQLEFNFCLWGRPA

SEQ ID NO: 557 - protein, 1093 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAGELADKKDRDASPSKEERKRSRTPDRERDRDRDRKSSPSKDRKRHRSRDRRRGGSRSRSRSRSKSAERERRHKERERDKERDRNKKDRDRDKDGHRRDKDRKRSSLSPGRGKDFKSRKDRDSKKDEEDEHGDKKPKAQPLSLEELLAKKKAEEEAEAKPKFLSKAEREAEALKRRQQEVEERQRMLEEERKKRKQFQDLGRKMLEDPQERERRERRERMERETNGNEDEEGRQKIREEKDKSKELHAIKERYLGGIKKRRRTRHLNDRKFVFEWDASEDTSIDYNPLYKERHQVQLLGRGFIAGIDLKQQKREQSRFYGDLMEKRRTLEEKEQEEARLRKLRKKEAKQRWDDRHWSQKKLDEMTDRDWRIFREDYSITTKGGKIPNPIRSWKDSSLPPHILEVIDKCGYKEPTPIQRQAIPIGLQNRDIIGVAETGSGKTAAFLIPLLVWITTLPKIDRIEESDQGPYAIILAPTRELAQQIEEETIKFGKPLGIRTVAVIGGISREDQGFRLRMGCEIVIATPGRLIDVLENRYLVLSRCTYVVLDEADRMIDMGFEPDVQKILEHMPVSNQKPDTDEAEDPEKMLANFESGKHKYRQTVMFTATMPPAVERLARSYLRRPAVVYIGSAGKPHERVEQKVFLMSESEKRKKLLAILEQGFDPPIIIFVNQKKGCDVLAKSLEKMGYNACTLHGGKGQEQREFALSNLKAGAKDILVATDVAGRGIDIQDVSMVVNYDMAKNIEDYIHRIGRTGRAGKSGVAITFLTKEDSAVFYELKQAILESPVSSCPPELANHPDAQHKPGTILTKKRREETIFANPAFLYKVVKASS

SEQ ID NO: 558 - protein, 820 aa, Homo sapiens
MAGELADKKDRDASPSKEERKRSRTPDRERDRDRDRKSSPSKDRKRHRSRDRRRGGSRSRSRSRSKSAERERRHKERERDKERDRNKKDRDRDKDGHRRDKDRKRSSLSPGRGKDFKSRKDRDSKKDEEDEHGDKKPKAQPLSLEELLAKKKAEEEAEAKPKFLSKAEREAEALKRRQQEVEERQRMLEEERKKRKQFQDLGRKMLEDPQERERRERRERMERETNGNEDEEGRQKIREEKDKSKELHAIKERYLGGIKKRRRTRHLNDRKFVFEWDASEDTSIDYNPLYKERHQVQLLGRGFIAGIDLKQQKREQSRFYGDLMEKRRTLEEKEQEEARLRKLRKKEAKQRWDDRHWSQKKLDEMTDRDWRIFREDYSITTKGGKIPNPIRSWKDSSLPPHILEVIDKCGYKEPTPIQRQAIPIGLQNRDIIGVAETGSGKTAAFLIPLLVWITTLPKIDRIEESDQGPYAIILAPTRELAQQIEEETIKFGKPLGIRTVAVIGGISREDQGFRLRMGCEIVIATPGRLIDVLENRYLVLSRCTYVVLDEADRMIDMGFEPDVQKILEHMPVSNQKPDTDEAEDPEKMLANFESGKHKYRQTVMFTATMPPAVERLARSYLRRPAVVYIGSAGKPHERVEQKVFLMSESEKRKKLLAILEQGFDPPIIIFVNQKKGCDVLAKSLEKMGYNACTLHGGKGQEQREFALSNLKAGAKDILVATDVAGRGIDIQDVSMVVNYDMAKNIEDYIHRIGRTGRAGKSGVAITFLTKEDSAVFYELKQAILESPVSSCPPELANHPDAQHKPGTILTKKRREETIFA

SEQ ID NO: 559 - protein, 494 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMEQRPRGCAAVAAALLLVLLGARAQGGTRSPRCDCAGDFHKKIGLFCCRGCPADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 560 - protein, 234 aa, Homo sapiens
MEQRPRGCAAVAAALLLVLLGARAQGGTRSPRCDCAGDFHKKIGLFCCRGCPADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 561 - protein, 849 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMNKSQEQVSFKDVCVDFTKEEWYLLDPAQKVLYRDVILENYSNFVSVGHCVTKPDVIFKIEQGEDPWIPENGFQCHPERKWKVDDLLESNQENQDGHFWPLMFTNKITNLEGGGNTRETPSPDIDSVSSRSFLLKVCDSCEMNLKNVSAVIISKKNYSRKKLNEFNIHKKPLPDSRREKIPVGIKSCDYDGERDGVRLCRDLARPVSHQPFEYNETGQGFCEEVALCTSQNSQLGETLYKHTECGRTFVDSLKLSVSQKTRLHTELHECSICRKSFYVDLQFGHHTALSGSSPYERNEYEKLFCDGTAFIVHQEAYTRKIAQEYKVSNRTWGKPALFKHQIIQMGGKPYEYYENRKNFSKKSHPTQPRRPHVGEKTFECGECGKMFWEKSNLTQHQRTHTGEKPYECTECGKAFCQKPHLTNHQRTHTGEKPYECKQCGKTFCVKSNLTEHQRTHTGEKPYECNACRKSFCHRSALTVHQRTHTGEKPFICSECGKSFCVKSNLIVHQRTHTGEKPYKCNECGKTFCEKSALTKHQRTHTGEKPYECHVCGKTFSQRSVLTKHLRIHTRVKALLASNPAFLYKVVKASS

SEQ ID NO: 562 - protein, 576 aa, Homo sapiens
MNKSQEQVSFKDVCVDFTKEEWYLLDPAQKVLYRDVILENYSNFVSVGHCVTKPDVIFKIEQGEDPWIPENGFQCHPERKWKVDDLLESNQENQDGHFWPLMFTNKITNLEGGGNTRETPSPDIDSVSSRSFLLKVCDSCEMNLKNVSAVIISKKNYSRKKLNEFNIHKKPLPDSRREKIPVGIKSCDYDGERDGVRLCRDLARPVSHQPFEYNETGQGFCEEVALCTSQNSQLGETLYKHTECGRTFVDSLKLSVSQKTRLHTELHECSICRKSFYVDLQFGHHTALSGSSPYERNEYEKLFCDGTAFIVHQEAYTRKIAQEYKVSNRTWGKPALFKHQIIQMGGKPYEYYENRKNFSKKSHPTQPRRPHVGEKTFECGECGKMFWEKSNLTQHQRTHTGEKPYECTECGKAFCQKPHLTNHQRTHTGEKPYECKQCGKTFCVKSNLTEHQRTHTGEKPYECNACRKSFCHRSALTVHQRTHTGEKPFICSECGKSFCVKSNLIVHQRTHTGEKPYKCNECGKTFCEKSALTKHQRTHTGEKPYECHVCGKTFSQRSVLTKHLRIHTRVKALLAS

SEQ ID NO: 563 - protein, 1196 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMLRRGSQALRRFSTGRVYFKNKLKLALIGQSLFGQEVYSHLRKEGHRVVGVFTVPDKDGKADPLALAAEKDGTPVFKLPKWRVKGKTIKEVAEAYRSVGAELNVLPFCTQFIPMDIIDSPKHGSIIYHPSILPRHRGASAINWTLIMGDKKAGFSVFWADDGLDTGPILLQRSCDVEPNDTVDALYNRFLFPEGIKAMVEAVQLIADGKAPRIPQPEEGATYEGIQKKENAEISWDQSAEVLHNWIRGHDKVPGAWTEINGQMVTFYGSTLLNSSVPPGEPLEIKGAKKPGLVTKNGLVLFGNDGKALTVRNLQFEDGKMIPASQYFSTGETSVVELTAEEVKVAETIKVIWAGILSNVPIIEDSTDFFKSGASSMDVARLVEEIRQKCGGLQLQNEDVYMATKFEGFIQKVVRKLRGEDQEVELVVDYISKEVNEIMVKMPYQCFINGQFTDADDGKTYDTINPTDGSTICKVSYASLADVDKAVAAAKDAFENGEWGRMNARERGRLMYRLADLLEENQEELATIEALDSGAVYTLALKTHIGMSVQTFRYFAGWCDKIQGSTIPINQARPNRNLTFTKKEPLGVCAIIIPWNYPLMMLAWKSAACLAAGNTLVLKPAQVTPLTALKFAELSVKAGFPKGVINIIPGSGGIAGQRLSEHPDIRKLGFTGSTPIGKQIMKSCAVSNLKKVSLELGGKSPLIIFNDCELDKTVRMGMGAVFFNKGENCIAAGRLFVEESIHDEFVTRVVEEIKKMKIGDPLDRSTDHGPQNHKAHLEKLLQYCETGVKEGATLVYGGRQVQRPGFFMEPTVFTDVEDYMYLAKEESFGPIMVISKFQNGDIDGVLQRANSTEYGLASGVFTRDINKAMYVSEKLEAGTVFINTYNKTDVAAPFGGVKQSGFGKDLGEEALNEYLKTKTVTLEYNPAFLYKVVKASS

SEQ ID NO: 564 - protein, 923 aa, Homo sapiens
MLRRGSQALRRFSTGRVYFKNKLKLALIGQSLFGQEVYSHLRKEGHRVVGVFTVPDKDGKADPLALAAEKDGTPVFKLPKWRVKGKTIKEVAEAYRSVGAELNVLPFCTQFIPMDIIDSPKHGSIIYHPSILPRHRGASAINWTLIMGDKKAGFSVFWADDGLDTGPILLQRSCDVEPNDTVDALYNRFLFPEGIKAMVEAVQLIADGKAPRIPQPEEGATYEGIQKKENAEISWDQSAEVLHNWIRGHDKVPGAWTEINGQMVTFYGSTLLNSSVPPGEPLEIKGAKKPGLVTKNGLVLFGNDGKALTVRNLQFEDGKMIPASQYFSTGETSVVELTAEEVKVAETIKVIWAGILSNVPIIEDSTDFFKSGASSMDVARLVEEIRQKCGGLQLQNEDVYMATKFEGFIQKVVRKLRGEDQEVELVVDYISKEVNEIMVKMPYQCFINGQFTDADDGKTYDTINPTDGSTICKVSYASLADVDKAVAAAKDAFENGEWGRMNARERGRLMYRLADLLEENQEELATIEALDSGAVYTLALKTHIGMSVQTFRYFAGWCDKIQGSTIPINQARPNRNLTFTKKEPLGVCAIIIPWNYPLMMLAWKSAACLAAGNTLVLKPAQVTPLTALKFAELSVKAGFPKGVINIIPGSGGIAGQRLSEHPDIRKLGFTGSTPIGKQIMKSCAVSNLKKVSLELGGKSPLIIFNDCELDKTVRMGMGAVFFNKGENCIAAGRLFVEESIHDEFVTRVVEEIKKMKIGDPLDRSTDHGPQNHKAHLEKLLQYCETGVKEGATLVYGGRQVQRPGFFMEPTVFTDVEDYMYLAKEESFGPIMVISKFQNGDIDGVLQRANSTEYGLASGVFTRDINKAMYVSEKLEAGTVFINTYNKTDVAAPFGGVKQSGFGKDLGEEALNEYLKTKTVTLEY

SEQ ID NO: 565 - protein, 726 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLDGDHVTHPDFMLYDALDVVLYMDPMCLDAFPKLVCFKKRIEAIPQIDKYLKSSKYIAWPLQGWQATFGGGDHPPKSDLVPRGSPGISGGGGGILGRGSHHHHHHGGGITSLYKKAGTMAGAGGGGCPAGGNDFQWCFSQVKGAIDEDVAEADIISTVEFNYSGDLLATGDKGGRVVIFQREQENKSRPHSRGEYNVYSTFQSHEPEFDYLKSLEIEEKINKIRWLPQQNAAHFLLSTNDKTIKLWKISERDKRAEGYNLKDEDGRLRDPFRITALRVPILKPMDLMVEASPRRIFANAHTYHINSISVNSDHETYLSADDLRINLWHLEITDRSFNIVDIKPANMEELTEVITAAEFHPHQCNVFVYSSSKGTIRLCDMRSSALCDRHSKFFEEPEDPSSRSFFSEIISSISDVKFSHSGRYMMTRDYLSVKVWDLNMESRPVETHQVHEYLRSKLCSLYENDCIFDKFECCWNGSDSAIMTGSYNNFFRMFDRDTRRDVTLEASRESSKPRASLKPRKVCTGGKRRKDEISVDSLDFNKKILHTAWHPVDNVIAVAATNNLYIFQDKINNPAFLYKVVKASS

SEQ ID NO: 566 - protein, 453 aa, Homo sapiens
MAGAGGGGCPAGGNDFQWCFSQVKGAIDEDVAEADIISTVEFNYSGDLLATGDKGGRVVIFQREQENKSRPHSRGEYNVYSTFQSHEPEFDYLKSLEIEEKINKIRWLPQQNAAHFLLSTNDKTIKLWKISERDKRAEGYNLKDEDGRLRDPFRITALRVPILKPMDLMVEASPRRIFANAHTYHINSISVNSDHETYLSADDLRINLWHLEITDRSFNIVDIKPANMEELTEVITAAEFHPHQCNVFVYSSSKGTIRLCDMRSSALCDRHSKFFEEPEDPSSRSFFSEIISSISDVKFSHSGRYMMTRDYLSVKVWDLNMESRPVETHQVHEYLRSKLCSLYENDCIFDKFECCWNGSDSAIMTGSYNNFFRMFDRDTRRDVTLEASRESSKPRASLKPRKVCTGGKRRKDEISVDSLDFNKKILHTAWHPVDNVIAVAATNNLYIFQDKIN

(19) United States
(12) Patent Application Publication
(10) Pub. No.: US 2023/0141003 A1
(43) Pub. Date: May 11, 2023

(54) BASELINE SERUM AUTOANTIBODIES FOR PREDICTING RECURRENCE AND TOXICITY FOR IMMUNE CHECKPOINT BLOCKADE IN CANCER PATIENTS

(71) Applicant: New York University, New York, NY (US)

(72) Inventors: Iman Osman, Jersey City, NJ (US); Judy Zhong, New York, NY (US); Paul Johannet, Scarsdale, NY (US)

(21) Appl. No.: 18/054,043

(22) Filed: Nov. 9, 2022

Related U.S. Application Data
(60) Provisional application No. 63/277,279, filed on Nov. 9, 2021.
(60) Provisional application No. 63/277,336, filed on Nov. 9, 2021.

(51) Int. Cl. C07K 16/28; A61P 35/00

(52) U.S. Cl. CPC C07K 16/2818; A61P 35/00; A61K 2039/505

(57) ABSTRACT

Provided are compositions and methods for determining baseline serum autoantibodies (autoAbs) for use in patient selection and for treatment with immune checkpoint blockade agents to promote avoidance of development of severe immune related adverse events (IRAEs). The treatments include administering anti-cancer agents that are not predicted to cause toxicity to selected patients, and administering agents to reduce predicted toxicity in selected patients.

### CROSS REFERENCE TO RELATED APPLICATIONS

This application claims priority to U.S. provisional patent application No. 63/277,279, filed Nov. 9, 2021, and U.S. provisional patent application No. 63/277,336, filed Nov. 9, 2021, the disclosures of each of which are incorporated herein by reference.

### STATEMENT REGARDING FEDERALLY SPONSORED RESEARCH OR DEVELOPMENT

This invention was made with government support under R01 CA231295 awarded by the National Institutes of Health. The government has certain rights in the invention.

### SEQUENCE LISTING

The instant application contains a Sequence Listing which has been submitted in .xml format and is hereby incorporated by reference in its entirety. Said .xml file is named “058636 00562.xml”, was created on Nov. 7, 2022, and is 790,199 bytes in size.

### RELATED INFORMATION

Adjuvant immune checkpoint blockade (ICB) produces clinical benefit for a subset of resected melanoma patients, but many individuals develop disease recurrence, and a substantial proportion develop immune-related adverse events (irAEs) (Eggermont et al., 2016; Weber et al., 2017). Toxicity can be severe enough to necessitate the interruption or permanent discontinuation of immunotherapy and may require treatment with systemic immunosuppressive agents. Even with appropriate clinical management, irAEs can lead to lifelong secondary conditions or, in rare cases, death (Michot et al., 2016; Postow et al., 2018). Thus, there is an urgent need to identify biomarkers of immunotherapy response and toxicity. Ideally, there would be a single assay that simultaneously risk-stratifies patients according to their likelihood of suffering recurrence or developing irAEs, which would help optimize patient selection for treatment. In patients who are at high risk for developing severe irAEs but proceed with treatment, it would facilitate monitoring and enable early intervention should toxicities develop. Thus, there is an ongoing need for identification and uses of biomarkers to predict responses to immune therapy and provide medical interventions based on the identification. The present disclosure is pertinent to this need.

### BRIEF SUMMARY

The present disclosure provides compositions and methods for determining baseline serum autoantibodies (autoAbs) for use in patient selection in connection with treatment with immune checkpoint blockade agents. The patients can be stratified into categories that include being at high risk for cancer recurrence or not, and for predicting whether or not a patient will experience toxicity in response to immune checkpoint blockade therapy. The disclosure includes all compositions and methods that are used to assess the autoAbs. The disclosure also includes medical interventions to reduce the risk of recurrence of cancer, to reduce toxicity that is predicted to occur if immune blockade therapy is used, and to select patients who are candidates for immune blockade therapy with low risk of severe toxicity, recurrence, or a combination thereof. The disclosure includes administering the immune blockade therapy to selected patients, administering anti-cancer agents that are not predicted to cause toxicity to selected patients, and administering agents to reduce predicted toxicity in selected patients. Thus, the disclosure provides pretreatment autoAb profiles correlate with the development of severe immune related adverse events (IRAEs) and moreover that autoAb profiles can be used to predict disease recurrence following treatment with adjuvant immune checkpoint blockade. As such, the disclosure provides signatures of autoAbs that can be used to simultaneously predict immunotherapy response and toxicity.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. A schematic illustrating the experimental design and workflow. Patients from CheckMate 238 who received NIVO were randomly assigned to discovery and test sets in a ratio of 75% to 25%. Patients from CheckMate 915 who received NIVO constituted an independent validation set. Patients from CheckMate 238 who received IPI and patients from CheckMate 915 who received IPI+NIVO were each randomly assigned to discovery and test sets in a ratio of 75% to 25%. Baseline sera were profiled for autoAb using the HuProt v4.0 microarray from CDI Labs. Analysis of differential autoAb expression in the discovery sets was followed by recurrence and severe toxicity building, testing of the identified signatures, and additional independent validation for the NIVO patients.

FIG. 2. Kaplan-Meier estimates of recurrence-free survival and waterfall plots of irAEs. Kaplan-Meier estimates of recurrence-free survival were plotted for patients predicted to have high- versus low-risks of recurrence from the (A) NIVO test set, (B) NIVO validation set, (C) IPI test set, and (D) IPI+NIVO test set. The cutoffs were derived from the corresponding discovery set. Survival curves were compared using the log-rank test. Waterfall plots show the relationship between the predicted and actual development of severe toxicity for patients from the (E) NIVO test set, (F) NIVO validation set, (G) IPI test set, and (H) IPI+NIVO test set. Patients above and below the horizontal bars are the predicted high- and low-risks of severe irAEs. The horizontal bars were drawn at the cutoffs derived from the corresponding discovery set. The colors represented the observed outcomes (red: patients experienced severe irAE; blue: patients did not experience severe irAE). Predicted irAE scores between the patients experienced severe irAE and those who did not experience severe irAE were compared using the two-sample t-test.

FIG. 3. Stratification of patients from the NIVO validation set into four quadrants based on their predicted risk of disease recurrence and severe toxicity. The quadrants were divided by the cutoffs of the irAE prediction score (x-axis) and the recurrence prediction score (y-axis) from the NIVO discovery set. Patients were assigned to four quadrants (top left: high efficacy/low recurrence & low toxicity; top right: high efficacy/low recurrence & high toxicity; bottom right: low efficacy/high recurrence & high toxicity; and bottom left: low efficacy/high recurrence & low toxicity). Each point represents a patient in the NIVO validation set, with colors representing the observed severe toxicity outcomes (red: patients experienced severe irAE; blue: patients did not experience severe irAE) and shapes representing the observed recurrence outcomes (triangles: patients experienced severe irAE; blue: patients did not experience severe irAE). The text shown are the percentage of patients within each quadrant who developed each of the predicted outcomes. FIG. 5 illustrates the stratification of patients in the NIVO test set, IPI test set, and IPI+NIVO test set.

FIG. 4. Functional enrichment analysis of the significantly differentially expressed autoantibodies (DEA). Panel A shows the terms enriched across all six treatment and outcome combinations. Panels B and C show the enriched terms for the DEA associated with (Panel B) disease recurrence and (Panel C) severe toxicity. The significantly enriched pathways were hierarchically clustered into a tree based on Kappa-statistical similarities among their gene memberships. The heatmap cells are colored by their p-values, grey cells indicate the lack of enrichment for that term in the corresponding column.

FIG. 5 provides results for a nivolumab test.

FIG. 6 provides graphs showing results from a Nivolumab Test, Nivolumab Validation, an IPI test, and a Invo+Ipi Test Cohort.

FIG. 7 provides graphs showing results from a Nivolumab Test, Nivolumab Validation, an IPI test, and a Invo+Ipi Test Cohort.

FIG. 8 depicts overlap in the autoAbs that constituted the final prediction signatures.

FIG. 9 shows overlap in the differentially expressed autoantibodies (DEAs) associated with nivolumab recurrence and severe toxicity.

FIG. 10 provides Table 1A.

FIG. 11 provides Table 1B.

FIG. 12 provides Table 1C.

FIG. 13 provides Table 2.

FIG. 14 provides Supplemental Table 1.

FIG. 15 provides Supplemental Table 2.

FIG. 16 provides Supplemental Table 3.

FIG. 17 provides Supplemental Table 4.

FIG. 18 provides Supplemental Table 5.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein. All protein sequences described herein include all isoforms of such proteins, e.g., proteins made from splice variants, and proteins that may vary from individual to individual in certain amino acids. Thus, all proteins described herein include proteins that have from 90.0-99.9% identity across their entire lengths to such proteins. The amino acid or polynucleotide sequence as the case may be associated with each GenBank or other database accession number of this disclosure is incorporated herein by reference as presented in the database on the effective filing date of this application or patent.

Aspects of this disclosure include each protein described herein, and all combinations of such proteins, wherein one or more of the proteins are present in vitro and are in contact with a biological sample obtained from an individual who has cancer. In embodiments, the individual from whom a first sample was obtained was not treated with any checkpoint inhibitor before the sample was obtained. In embodiments, the individual from whom a first sample is obtained has been diagnosed with any type of cancer. In embodiments, the cancer is a solid or liquid tumor. In embodiments, the cancer is renal cell carcinoma, breast cancer, prostate cancer, pancreatic cancer, lung cancer, liver cancer, ovarian cancer, cervical cancer, colon cancer, esophageal cancer, stomach cancer, bladder cancer, brain cancer, testicular cancer, head and neck cancer, melanoma or another skin cancer, any sarcoma, including but not limited to fibrosarcoma, angiosarcoma, adenocarcinoma, and rhabdomyosarcoma, and any blood cancer, including all types of leukemia, lymphoma, and myeloma. In a non-limiting embodiment, the biological sample is obtained from an individual who has been diagnosed with melanoma and was not treated with any checkpoint inhibitor before the sample was obtained.

In embodiments, a second, third, fourth, sample, etc. can be obtained from an individual who is undergoing treatment and tested to monitor the effect of the treatment, and keep steady, change, adjust, or discontinue treatment with a checkpoint inhibitor. In embodiments, the treatment is adjusted to prevent or mitigate the onset of irAEs, such as by administering an agent to the individual as described further herein.

A method of the present disclosure comprises screening for the presence of one or more sets of antibodies in a biological sample (such as blood, serum, plasma etc.) from an individual who is being considered as a candidate for therapy with one or more immune checkpoint inhibitors, and based upon the antibody profile, identifying the appropriate immune checkpoint inhibitors for administration to the individual, or determining that the individual should not be treated with a checkpoint inhibitor, or determining that the immune checkpoint inhibitors should be administered in conjunction with toxicity mitigation agents/process. The checkpoint inhibitors may be anti-PD-1, anti-CTLA-4, or a combination thereof. In embodiments, the checkpoint inhibitors are Ipilimumab used as a single checkpoint inhibitor therapy, or Nivolumab as a single checkpoint inhibitor therapy, or a combination of Ipilimumab and Nivolumab as a combination checkpoint inhibitor therapy.

In non-limiting embodiments, the disclosure provides a set of distinct autoAb signatures that can be used to predict the following two treatment outcomes for patients with advanced melanoma who receive adjuvant immunotherapy: (i) severe (Grade 3 or 4) versus non-severe (Grade 1 or 2) immune related adverse events; and (ii) disease recurrence versus no disease recurrence. Methods of grading of immune related adverse events is known in the art and are described further below. The distinct signatures predict ipilimumab efficacy, ipilimumab toxicity, nivolumab efficacy, and nivolumab toxicity. The efficacy and toxicity signatures for each treatment are combined so that patients can be stratified into one of four predicted outcomes: (i) efficacy and no severe toxicity; (ii) efficacy and severe toxicity; (iii) no efficacy and no severe toxicity; and (iv) no efficacy and severe toxicity. In certain approaches, predicting an individual will not have recurrence of cancer after treatment with a checkpoint inhibitor indicates the checkpoint inhibitor, or combination of checkpoint inhibitors, will have efficacy.

The signatures are developed by analyzing pre-checkpoint inhibitor samples for binding to a plurality of proteins. In embodiments, the plurality of proteins used in the described compositions and methods are selected from Table X.

With respect to compositions and methods of this disclosure, antibodies, if present in the biological sample, bind with specificity to one or more proteins that are present in an assay that is designed to determine the presence, absence, and/or amount of such antibodies. Thus, in embodiments, the disclosure comprises exposing a biological sample to a protein array. In embodiments, the protein array comprises at least 50%, 60%, 70%, or 80% of the proteins in the human proteome. In embodiments, the protein array pertains to the proteins known as of the date of the filing of this application or patent. In embodiments, the protein array comprises at least one protein from Table X. In embodiments, the plurality of proteins attached to the substrate comprises fewer than 21,000 proteins. In embodiments, the plurality of proteins attached to the array comprise or consist of 1-283 proteins from Table X, inclusive, and including all numbers and ranges of numbers between 1-283. Thus, in embodiments, the only proteins in the plurality of proteins attached to the substrate are selected from the proteins of Table X. In embodiments, the array comprises all of the proteins described in Table X.

Table X includes Tables A, B, C, D, E, and F. When reference to Table X is made, unless stated otherwise, all of Tables A, B, C, D, E, and F are included. Each of Tables A, B, C, D, E, and F include a plurality of proteins and an indication of what the auto-antibody signature prediction associated with auto-antibodies to each protein is. Specifically, Table A refers to proteins used for measuring autoantibodies to predict cancer recurrence when a patient treated with Ipilimumab as a monotherapy. Table B refers to proteins used for measuring autoantibodies to predict severe toxicity when a patient is treated with Ipilimumab as the only checkpoint inhibitor. Table C refers to proteins used for measuring autoantibodies to predict cancer recurrence when a patient is treated with Nivolumab as the only checkpoint inhibitor. Table D refers to proteins used for measuring autoantibodies to predict severe toxicity when a patient is treated with Nivolumab as the only checkpoint inhibitor. Table E refers to proteins used for measuring autoantibodies to predict cancer recurrence when a patient treated with Ipilimumab and Nivolumab as a combination therapy. Table F refers to proteins used for measuring autoantibodies to predict severe toxicity when a patient is treated with Ipilimumab and Nivolumab as a combination therapy.

Thus, the disclosure provides one or more arrays that comprise substrates with one or more of the described proteins in Table X that are reversibly or irreversibly attached to the substrate. The disclosure includes the described proteins and substrates that are in contact with a sample from an individual who has cancer, such as melanoma. The disclosure also includes the described protein-substrate combinations wherein auto-antibodies in the patient sample are bound to at least some of the proteins on the substrate.

TABLE X
Protein Signature Tables

| Protein | Signature |
|---|---|
| Table A | |
| OXSR1 | Ipilimumab: recurrence |
| TRIM5 | Ipilimumab: recurrence |
| TRIM5 | Ipilimumab: recurrence |
| RBM33 | Ipilimumab: recurrence |
| MAGEA4 | Ipilimumab: recurrence |
| SLC43A2 | Ipilimumab: recurrence |
| CELA3B | Ipilimumab: recurrence |
| MAGEA11 | Ipilimumab: recurrence |
| ECSIT | Ipilimumab: recurrence |
| C7orf25 | Ipilimumab: recurrence |
| MYOG | Ipilimumab: recurrence |
| GNPAT | Ipilimumab: recurrence |
| SELPLG | Ipilimumab: recurrence |
| SPACA1 | Ipilimumab: recurrence |
| BNIPL | Ipilimumab: recurrence |
| MFSD9 | Ipilimumab: recurrence |
| ZFYVE19 | Ipilimumab: recurrence |
| SESN3 | Ipilimumab: recurrence |
| CALHM1 | Ipilimumab: recurrence |
| DAD1 | Ipilimumab: recurrence |
| UFSP2 | Ipilimumab: recurrence |
| CDCA8 | Ipilimumab: recurrence |
| TMEM204 | Ipilimumab: recurrence |
| DIABLO | Ipilimumab: recurrence |
| PHTF2_frag | Ipilimumab: recurrence |
| PSKH1 | Ipilimumab: recurrence |
| ASPH | Ipilimumab: recurrence |
| ZNF710 | Ipilimumab: recurrence |
| RHBDF2 | Ipilimumab: recurrence |
| SLC6A11 | Ipilimumab: recurrence |
| ASCL1 | Ipilimumab: recurrence |
| CNP | Ipilimumab: recurrence |
| PPP1R12B | Ipilimumab: recurrence |
| NEDD4L | Ipilimumab: recurrence |
| NLK | Ipilimumab: recurrence |
| ARHGEF3 | Ipilimumab: recurrence |
| SKAP1 | Ipilimumab: recurrence |
| RNF8 | Ipilimumab: recurrence |
| ZBTB12 | Ipilimumab: recurrence |
| ATP6V1G3 | Ipilimumab: recurrence |
| C2orf66 | Ipilimumab: recurrence |
| SCHIP1 | Ipilimumab: recurrence |
| BEND6 | Ipilimumab: recurrence |
| MMP3 | Ipilimumab: recurrence |
| PAK2 | Ipilimumab: recurrence |
| RRM1 | Ipilimumab: recurrence |
| PLEKHG6 | Ipilimumab: recurrence |
| PRMT8 | Ipilimumab: recurrence |
| RBAK | Ipilimumab: recurrence |
| UGT2B15 | Ipilimumab: recurrence |
| RAET1L | Ipilimumab: recurrence |
| OGDH | Ipilimumab: recurrence |
| HSFX1 | Ipilimumab: recurrence |
| FAN1 | Ipilimumab: recurrence |
| PAK1 | Ipilimumab: recurrence |
| OR2B11 | Ipilimumab: recurrence |
| Table B | |
| TMEM147 | Ipilimumab: severe toxicity |
| PLGRKT | Ipilimumab: severe toxicity |
| SCAMP1 | Ipilimumab: severe toxicity |
| ASCC1 | Ipilimumab: severe toxicity |
| C10orf54 | Ipilimumab: severe toxicity |
| C21orf33 | Ipilimumab: severe toxicity |
| SPATC1L | Ipilimumab: severe toxicity |
| METTL9 | Ipilimumab: severe toxicity |
| CXCL16 | Ipilimumab: severe toxicity |
| SPX | Ipilimumab: severe toxicity |
| TRIM48 | Ipilimumab: severe toxicity |
| CFAP61 | Ipilimumab: severe toxicity |
| IQCB1 | Ipilimumab: severe toxicity |
| IGHA1 | Ipilimumab: severe toxicity |
| ATF5 | Ipilimumab: severe toxicity |
| ALDH3A1 | Ipilimumab: severe toxicity |
| SPON2 | Ipilimumab: severe toxicity |
| PLAUR | Ipilimumab: severe toxicity |
| CALHM1 | Ipilimumab: severe toxicity |
| NUP62 | Ipilimumab: severe toxicity |
| KCNA2 | Ipilimumab: severe toxicity |
| SLC23A1 | Ipilimumab: severe toxicity |
| SLC34A1 | Ipilimumab: severe toxicity |
| TRNAU1AP | Ipilimumab: severe toxicity |
| KJ903238 | Ipilimumab: severe toxicity |
| RAB27A | Ipilimumab: severe toxicity |
| CCNG2 | Ipilimumab: severe toxicity |
| BDNF | Ipilimumab: severe toxicity |
| BOP1 | Ipilimumab: severe toxicity |
| ASB6 | Ipilimumab: severe toxicity |
| LONRF2 | Ipilimumab: severe toxicity |
| PDXDC1 | Ipilimumab: severe toxicity |
| IFNA8 | Ipilimumab: severe toxicity |
| MIR1-1HG | Ipilimumab: severe toxicity |
| COLEC10 | Ipilimumab: severe toxicity |
| SIRPA | Ipilimumab: severe toxicity |
| CPSF3 | Ipilimumab: severe toxicity |
| MYCN | Ipilimumab: severe toxicity |
| COX5A | Ipilimumab: severe toxicity |
| ITGA5 | Ipilimumab: severe toxicity |
| MAX | Ipilimumab: severe toxicity |
| NDRG3 | Ipilimumab: severe toxicity |
| BDNF | Ipilimumab: severe toxicity |
| IFITM3 | Ipilimumab: severe toxicity |
| ANKDD1A | Ipilimumab: severe toxicity |
| MAGEB10 | Ipilimumab: severe toxicity |
| TTC6 | Ipilimumab: severe toxicity |
| CELA1 | Ipilimumab: severe toxicity |
| ACER2 | Ipilimumab: severe toxicity |
| STX16 | Ipilimumab: severe toxicity |
| IGL@ | Ipilimumab: severe toxicity |
| PRTN3 | Ipilimumab: severe toxicity |
| SUCNR1 | Ipilimumab: severe toxicity |
| CSTL1 | Ipilimumab: severe toxicity |
| TNFRSF25 | Ipilimumab: severe toxicity |
| TNFRSF25 | Ipilimumab: severe toxicity |
| LZTR1 | Ipilimumab: severe toxicity |
| PIWIL2 | Ipilimumab: severe toxicity |
| ZNF521 | Ipilimumab: severe toxicity |
| ANP32C | Ipilimumab: severe toxicity |
| AQP11 | Ipilimumab: severe toxicity |
| MRPL39 | Ipilimumab: severe toxicity |
| NR2F6 | Ipilimumab: severe toxicity |
| ATP6V0A4 | Ipilimumab: severe toxicity |
| KCND2 | Ipilimumab: severe toxicity |
| OR11H12 | Ipilimumab: severe toxicity |
| Table C | |
| IGIP | Nivolumab: recurrence |
| EMC2 | Nivolumab: recurrence |
| C2orf15 | Nivolumab: recurrence |
| CLPS | Nivolumab: recurrence |
| CCDC107 | Nivolumab: recurrence |
| COX16 | Nivolumab: recurrence |
| TWF1 | Nivolumab: recurrence |
| SLCO2A1 | Nivolumab: recurrence |
| CDC23 | Nivolumab: recurrence |
| HIST1H3A | Nivolumab: recurrence |
| HIST1H3A | Nivolumab: recurrence |
| KJ904142_frag | Nivolumab: recurrence |
| PNPLA3 | Nivolumab: recurrence |
| MMP3 | Nivolumab: recurrence |
| LINC01554 | Nivolumab: recurrence |
| TNFSF11 | Nivolumab: recurrence |
| SLC22A12 | Nivolumab: recurrence |
| TTC39B | Nivolumab: recurrence |
| CCNT1 | Nivolumab: recurrence |
| PSMG1 | Nivolumab: recurrence |
| DEPTOR | Nivolumab: recurrence |
| MS4A3 | Nivolumab: recurrence |
| OLFM1 | Nivolumab: recurrence |
| TMEM116 | Nivolumab: recurrence |
| NDUFA5 | Nivolumab: recurrence |
| NDUFA5 | Nivolumab: recurrence |
| SCUBE1 | Nivolumab: recurrence |
| CDC27 | Nivolumab: recurrence |
| DEFB129 | Nivolumab: recurrence |
| TBP | Nivolumab: recurrence |
| TAF6 | Nivolumab: recurrence |
| Table D | |
| SLC30A5 | Nivolumab: severe toxicity |
| ELMOD3 | Nivolumab: severe toxicity |
| SH3BP5 | Nivolumab: severe toxicity |
| ACRV1 | Nivolumab: severe toxicity |
| CD300LB | Nivolumab: severe toxicity |
| PSMA4 | Nivolumab: severe toxicity |
| BCAS3_frag | Nivolumab: severe toxicity |
| HAAO | Nivolumab: severe toxicity |
| CHIC2 | Nivolumab: severe toxicity |
| CHIC2 | Nivolumab: severe toxicity |
| TMEM31 | Nivolumab: severe toxicity |
| EEF1G | Nivolumab: severe toxicity |
| METTL13 | Nivolumab: severe toxicity |
| SFXN1 | Nivolumab: severe toxicity |
| HSD3B1 | Nivolumab: severe toxicity |
| TMEM106B | Nivolumab: severe toxicity |
| CAPS2 | Nivolumab: severe toxicity |
| ABHD1 | Nivolumab: severe toxicity |
| OGFR | Nivolumab: severe toxicity |
| EPB41L1 | Nivolumab: severe toxicity |
| KRT33B | Nivolumab: severe toxicity |
| NTSR2 | Nivolumab: severe toxicity |
| SLC22A6 | Nivolumab: severe toxicity |
| CSK | Nivolumab: severe toxicity |
| SPINK7 | Nivolumab: severe toxicity |
| JADE3 | Nivolumab: severe toxicity |
| HYAL1 | Nivolumab: severe toxicity |
| MRAS | Nivolumab: severe toxicity |
| TOP3B | Nivolumab: severe toxicity |
| GTPBP6 | Nivolumab: severe toxicity |
| ZNF254 | Nivolumab: severe toxicity |
| ZNF624 | Nivolumab: severe toxicity |
| TTC31 | Nivolumab: severe toxicity |
| TSFM | Nivolumab: severe toxicity |
| MSGN1 | Nivolumab: severe toxicity |
| ARHGAP8 | Nivolumab: severe toxicity |
| TNFRSF25 | Nivolumab: severe toxicity |
| MARK4 | Nivolumab: severe toxicity |
| SV2A | Nivolumab: severe toxicity |
| TGFBRAP1 | Nivolumab: severe toxicity |
| DEFB109P1 | Nivolumab: severe toxicity |
| SPANXA1 | Nivolumab: severe toxicity |
| CYP11B2 | Nivolumab: severe toxicity |
| PIGR | Nivolumab: severe toxicity |
| MMP11 | Nivolumab: severe toxicity |
| Nol3 | Nivolumab: severe toxicity |
| ECD | Nivolumab: severe toxicity |
| RASEF | Nivolumab: severe toxicity |
| Table E | |
| SRPX2 | Ipilimumab plus nivolumab: recurrence |
| PANK1_frag | Ipilimumab plus nivolumab: recurrence |
| WNT7A | Ipilimumab plus nivolumab: recurrence |
| DIDO1 | Ipilimumab plus nivolumab: recurrence |
| FAM156A | Ipilimumab plus nivolumab: recurrence |
| MAGEA3 | Ipilimumab plus nivolumab: recurrence |
| MAGEA3 | Ipilimumab plus nivolumab: recurrence |
| CD177 | Ipilimumab plus nivolumab: recurrence |
| NUP133 | Ipilimumab plus nivolumab: recurrence |
| ZNF622 | Ipilimumab plus nivolumab: recurrence |
| UBA6 | Ipilimumab plus nivolumab: recurrence |
| ERCC8 | Ipilimumab plus nivolumab: recurrence |
| CBX3 | Ipilimumab plus nivolumab: recurrence |
| SIK1 | Ipilimumab plus nivolumab: recurrence |
| LILRB5 | Ipilimumab plus nivolumab: recurrence |
| AGMAT | Ipilimumab plus nivolumab: recurrence |
| FJX1 | Ipilimumab plus nivolumab: recurrence |
| CTDSPL2 | Ipilimumab plus nivolumab: recurrence |
| NCBP2-AS2 | Ipilimumab plus nivolumab: recurrence |
| HTR4 | Ipilimumab plus nivolumab: recurrence |
| LOC102724398 | Ipilimumab plus nivolumab: recurrence |
| ISL1 | Ipilimumab plus nivolumab: recurrence |
| CELA2B | Ipilimumab plus nivolumab: recurrence |
| MIER2 | Ipilimumab plus nivolumab: recurrence |
| MIER2 | Ipilimumab plus nivolumab: recurrence |
| RTFDC1 | Ipilimumab plus nivolumab: recurrence |
| LOC440337 | Ipilimumab plus nivolumab: recurrence |
| SUSD3 | Ipilimumab plus nivolumab: recurrence |
| MESP2 | Ipilimumab plus nivolumab: recurrence |
| ATP1B4 | Ipilimumab plus nivolumab: recurrence |
| C1orf61 | Ipilimumab plus nivolumab: recurrence |
| TTLL13P | Ipilimumab plus nivolumab: recurrence |
| TTL | Ipilimumab plus nivolumab: recurrence |
| CDKL2 | Ipilimumab plus nivolumab: recurrence |
| Cebpa | Ipilimumab plus nivolumab: recurrence |
| ZNF646 | Ipilimumab plus nivolumab: recurrence |
| Sebox | Ipilimumab plus nivolumab: recurrence |
| TCF19 | Ipilimumab plus nivolumab: recurrence |
| C3orf58 | Ipilimumab plus nivolumab: recurrence |
| SH3RF2 | Ipilimumab plus nivolumab: recurrence |
| C15orf53 | Ipilimumab plus nivolumab: recurrence |
| ZNF726 | Ipilimumab plus nivolumab: recurrence |
| Table F | |
| RGR | Ipilimumab plus nivolumab: severe toxicity |
| SGTA | Ipilimumab plus nivolumab: severe toxicity |
| NCAPG_frag | Ipilimumab plus nivolumab: severe toxicity |
| C10orf53 | Ipilimumab plus nivolumab: severe toxicity |
| PDP2 | Ipilimumab plus nivolumab: severe toxicity |
| SERPINF2 | Ipilimumab plus nivolumab: severe toxicity |
| OSGIN1 | Ipilimumab plus nivolumab: severe toxicity |
| SYT11 | Ipilimumab plus nivolumab: severe toxicity |
| ARL14EP | Ipilimumab plus nivolumab: severe toxicity |
| GBA | Ipilimumab plus nivolumab: severe toxicity |
| DHX29 | Ipilimumab plus nivolumab: severe toxicity |
| CAPN7 | Ipilimumab plus nivolumab: severe toxicity |
| CCL17 | Ipilimumab plus nivolumab: severe toxicity |
| GPR176 | Ipilimumab plus nivolumab: severe toxicity |
| TDRD3 | Ipilimumab plus nivolumab: severe toxicity |
| CDC23 | Ipilimumab plus nivolumab: severe toxicity |
| SCARA5 | Ipilimumab plus nivolumab: severe toxicity |
| TES | Ipilimumab plus nivolumab: severe toxicity |
| FBXO3 | Ipilimumab plus nivolumab: severe toxicity |
| BUB3 | Ipilimumab plus nivolumab: severe toxicity |
| PRSS3 | Ipilimumab plus nivolumab: severe toxicity |
| FAM96B | Ipilimumab plus nivolumab: severe toxicity |
| ARTN | Ipilimumab plus nivolumab: severe toxicity |
| GDF9 | Ipilimumab plus nivolumab: severe toxicity |
| NOXA1 | Ipilimumab plus nivolumab: severe toxicity |
| SLC25A43 | Ipilimumab plus nivolumab: severe toxicity |
| ACTRT2 | Ipilimumab plus nivolumab: severe toxicity |
| ACVR2A | Ipilimumab plus nivolumab: severe toxicity |
| CBFA2T3 | Ipilimumab plus nivolumab: severe toxicity |
| MFAP5 | Ipilimumab plus nivolumab: severe toxicity |
| ZHX3 | Ipilimumab plus nivolumab: severe toxicity |
| KBTBD12 | Ipilimumab plus nivolumab: severe toxicity |
| PTEN | Ipilimumab plus nivolumab: severe toxicity |
| DYNC1H1 | Ipilimumab plus nivolumab: severe toxicity |
| PRAMEF3 | Ipilimumab plus nivolumab: severe toxicity |
| DDX23 | Ipilimumab plus nivolumab: severe toxicity |
| TNFRSF25 | Ipilimumab plus nivolumab: severe toxicity |
| Q640n4 | Ipilimumab plus nivolumab: severe toxicity |
| ALDH1L2 | Ipilimumab plus nivolumab: severe toxicity |
| PPP2R2D | Ipilimumab plus nivolumab: severe toxicity |

Table X relates to Table Y. Table Y provides amino acid sequence information for the described proteins. The left column of Table Y provides modified sequences that were used in embodiments of the disclosure. The right hand column provides unmodified human protein sequences that may also be used in embodiments of the disclosure.

TABLE Y

| Seq | | | | | | |
|---|---|---|---|---|---|---|
| ID | | | | Pro- | | |
| No | JHU | Sig | coef | tein | Sequence | Sequence information |
| 1 | JHUO | IPI_ | 0.402821 | OXSR1 | MSPILGYWKIKGLVQPTRLLLEYLEEKYEEHLYER | Full Literal Sequence- |
| | 0147 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSEDSSALPWSINR | |
| | | | | | DDYELQEVIGSGATAVVQAAYCAPKKEKVAIKRIN | |
| | | | | | LEKCQTSMDELLKEIQAMSQCHHPNIVSYYTSFV | |
| | | | | | VKDELWLVMKLLSGGSVLDIIKHIVAKGEHKSGVL | |
| | | | | | DESTIATILREVLEGLEYLHKNGQIHRDVKAGNILL | |
| | | | | | GEDGSVQIADFGVSAFLATGGDITRNKVRKTFVG | |
| | | | | | TPCWMAPEVMEQVRGYDFKADIWSFGITAIELAT | |
| | | | | | GAAPYHKYPPMKVLMLTLQNDPPSLETGVQDKE | |
| | | | | | MLKKYGKSFRKMISLCLQKDPEKRPTAAELLRHK | |
| | | | | | FFQKAKNKEFLQEKTLQRAPTISERAKKVRRVPG | |
| | | | | | SSGRLHKTEDGGWEWSDDEFDEESEEGKAAISQ | |
| | | | | | LRSPRVKESISNSELFPTTDPVGTLLQVPEQISAH | |
| | | | | | LPQPAGQIATQPTQVSLPPTAEPAKTAQALSSGS | |
| | | | | | GSQETKIPISLVLRLRNSKKELNDIRFEFTPGRDTA | |
| | | | | | EGVSQELISAGLVDGRDLVIVAANLQKIVEEPQSN | |
| | | | | | RSVTFKLASGVEGSDIPDDGKLIGFAQLSIS | |
| 2 | JHUO | IPI_ | 0.402821 | OXSR1 | MSEDSSALPWSINRDDYELQEVIGSGATAVVQAA | Core Human ORF Only |
| | 0147 | RFS | | | YCAPKKEKVAIKRINLEKCQTSMDELLKEIQAMSQ | |
| | | | | | CHHPNIVSYYTSFVVKDELWLVMKLLSGGSVLDII | |
| | | | | | KHIVAKGEHKSGVLDESTIATILREVLEGLEYLHKN | |
| | | | | | GQIHRDVKAGNILLGEDGSVQIADFGVSAFLATG | |
| | | | | | GDITRNKVRKTFVGTPCWMAPEVMEQVRGYDFK | |
| | | | | | ADIWSFGITAIELATGAAPYHKYPPMKVLMLTLQN | |
| | | | | | DPPSLETGVQDKEMLKKYGKSFRKMISLCLQKDP | |
| | | | | | EKRPTAAELLRHKFFQKAKNKEFLQEKTLQRAPTI | |
| | | | | | SERAKKVRRVPGSSGRLHKTEDGGWEWSDDEF | |
| | | | | | DEESEEGKAAISQLRSPRVKESISNSELFPTTDPV | |
| | | | | | GTLLQVPEQISAHLPQPAGQIATQPTQVSLPPTAE | |
| | | | | | PAKTAQALSSGSGSQETKIPISLVLRLRNSKKELN | |
| | | | | | DIRFEFTPGRDTAEGVSQELISAGLVDGRDLVIVA | |
| | | | | | ANLQKIVEEPQSNRSVTFKLASGVEGSDIPDDGK | |
| | | | | | LIGFAQLSIS | |
| 3 | JHUO | IPL | 0.44074507 | TRIM5 | MSPILGYWKIKGLVQPTRLLLEYLEEKYEEHLYER | Full Literal Sequence- |
| | 0379 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASGILVNVKEEVTC | |
| | | | | | PICLELLTQPLSLDCGHSFCQACLTANHKKSMLD | |
| | | | | | KGESSCPVCRISYQPENIRPNRHVANIVEKLREVK | |
| | | | | | LSPEGQKVDHCARHGEKLLLFCQEDGKVICWLC | |
| | | | | | ERSQEHRGHHTFLTEEVAREYQVKLQAALEMLR | |
| | | | | | QKQQEAEELEADIREEKASWKTQIQYDKTNVLAD | |
| | | | | | FEQLRDILDWEESNELQNLEKEEEDILKSLTNSET | |
| | | | | | EMVQQTQSLRELISDLEHRLQGSVMELLQGVDG | |
| | | | | | VIKRTENVTLKKPETFPKNQRRVFRAPDLKGMLE | |
| | | | | | VFRELTDVRRYWGKEKSHYHKPPCGLSLLLSLSF | |
| | | | | | RILCSLLGSCFKIYDSPSKTHITYPSL | |
| 4 | JHUO | IPL | 0.44074507 | TRIM5 | MASGILVNVKEEVTCPICLELLTQPLSLDCGHSFC | Core Human ORF Only |
| | 0379 | RFS | | | QACLTANHKKSMLDKGESSCPVCRISYQPENIRP | |
| | | | | | NRHVANIVEKLREVKLSPEGQKVDHCARHGEKLL | |
| | | | | | LFCQEDGKVICWLCERSQEHRGHHTFLTEEVAR | |
| | | | | | EYQVKLQAALEMLRQKQQEAEELEADIREEKAS | |
| | | | | | WKTQIQYDKTNVLADFEQLRDILDWEESNELQNL | |
| | | | | | EKEEEDILKSLTNSETEMVQQTQSLRELISDLEHR | |
| | | | | | LQGSVMELLQGVDGVIKRTENVTLKKPETFPKNQ | |
| | | | | | RRVFRAPDLKGMLEVFRELTDVRRYWGKEKSHY | |
| | | | | | HKPPCGLSLLLSLSFRILCSLLGSCFKIYDSPSKTH | |
| | | | | | ITYPSL | |
| 5 | JHUO | IPL | 0.140784934 | TRIM5 | MSPILGYWKIKGLVQPTRLLLEYLEEKYEEHLYER | Full Literal Sequence- |
| | 0379 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASGILVNVKEEVTC | |
| | | | | | PICLELLTQPLSLDCGHSFCQACLTANHKKSMLD | |
| | | | | | KGESSCPVCRISYQPENIRPNRHVANIVEKLREVK | |
| | | | | | LSPEGQKVDHCARHGEKLLLFCQEDGKVICWLC | |
| | | | | | ERSQEHRGHHTFLTEEVAREYQVKLQAALEMLR | |
| | | | | | QKQQEAEELEADIREEKASWKTQIQYDKTNVLAD | |
| | | | | | FEQLRDILDWEESNELQNLEKEEEDILKSLTNSET | |
| | | | | | EMVQQTQSLRELISDLEHRLQGSVMELLQGVDG | |
| | | | | | VIKRTENVTLKKPETFPKNQRRVFRAPDLKGMLE | |
| | | | | | VFRELTDVRRYWGKEKSHYHKPPCGLSLLLSLSF | |
| | | | | | RILCSLLGSCFKIYDSPSKTHITYPSL | |
| 6 | JHUO | IPL | 0.140784934 | TRIM5 | MASGILVNVKEEVTCPICLELLTQPLSLDCGHSFC | Core Human ORF Only |
| | 0379 | RFS | | | QACLTANHKKSMLDKGESSCPVCRISYQPENIRP | |
| | | | | | NRHVANIVEKLREVKLSPEGQKVDHCARHGEKLL | |
| | | | | | LFCQEDGKVICWLCERSQEHRGHHTFLTEEVAR | |
| | | | | | EYQVKLQAALEMLRQKQQEAEELEADIREEKAS | |
| | | | | | WKTQIQYDKTNVLADFEQLRDILDWEESNELQNL | |
| | | | | | EKEEEDILKSLTNSETEMVQQTQSLRELISDLEHR | |
| | | | | | LQGSVMELLQGVDGVIKRTENVTLKKPETFPKNQ | |
| | | | | | RRVFRAPDLKGMLEVFRELTDVRRYWGKEKSHY | |
| | | | | | HKPPCGLSLLLSLSFRILCSLLGSCFKIYDSPSKTH | |
| | | | | | ITYPSL | |
| 7 | JHUO | IPL | 0.613459403 | RBM33 | MSPILGYWKIKGLVQPTRLLLEYLEEKYEEHLYER | Full Literal Sequence- |
| | 0809 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAALGASGGAGAG | |
| | | | | | DDDFDQFDKPGAERSWRRRAADEDWDSELEDD | |
| | | | | | LLGEDLLSGKKNQSDLSDEELNDDLLQSDNEDEE | |
| | | | | | NFSSQGVTISLNATSGMVTSFELSDNTNDQSGEQ | |
| | | | | | ESEYEQEQGEDELVYHKSDGSELYTQEYPEEGQ | |
| | | | | | YEGHEAELTEDQIEYVEEPEEEQLYTDEVLDIEIN | |
| | | | | | EPLDEFTGGMETLELQKDIKEESDEEEEDDEESG | |
| | | | | | RLRFKTERKEGTIIRLSDVTRERRNIPETLGNFFAC | |
| | | | | | LPSSFTLISTSSIVLL | |
| 8 | JHUO | IPL | 0.613459403 | RBM33 | MAAALGASGGAGAGDDDFDQFDKPGAERSWRR | Core Human ORF Only |
| | 0809 | RFS | | | RAADEDWDSELEDDLLGEDLLSGKKNQSDLSDE | |
| | | | | | ELNDDLLQSDNEDEENFSSQGVTISLNATSGMVT | |
| | | | | | SFELSDNTNDQSGEQESEYEQEQGEDELVYHKS | |
| | | | | | DGSELYTQEYPEEGQYEGHEAELTEDQIEYVEEP | |
| | | | | | EEEQLYTDEVLDIEINEPLDEFTGGMETLELQKDIK | |
| | | | | | EESDEEEEDDEESGRLRFKTERKEGTIIRLSDVTR | |
| | | | | | ERRNIPETLGNFFACLPSSFTLISTSSIVLL | |
| 9 | JHUO | IPL | 0.1056821 | MAGE | MSPILGYWKIKGLVQPTRLLLEYLEEKYEEHLYER | Full Literal Sequence- |
| | 1014 | RFS | 46 | A4 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSSEQKSQHCKPEE | |
| | | | | | GVEAQEEALGLVGAQAPTTEEQEAAVSSSSPLVP | |
| | | | | | GTLEEVPAAESAGPPQSPQGASALPTTISFTCWR | |
| | | | | | QPNEGSSSQEEEGPSTSPDAESLFREALSNKVD | |
| | | | | | ELAHFLLRKYRAKELVTKAEMLERVIKNYKRCFPV | |
| | | | | | IFGKASESLKMIFGIDVKEVDPTSNTYTLVTCLGLS | |
| | | | | | YDGLLGNNQIFPKTGLLIIVLGTIAMEGDSASEEEI | |
| | | | | | WEELGVMGVYDGREHTVYGEPRKLLTQDWVQE | |
| | | | | | NYLEYRQVPGSNPARYEFLWGPRALAETSYVKV | |
| | | | | | LEHVVRVNARVRIAYPSLREAALLEEEEGV | |
| 10 | JHUO | IPL | 0.105682146 | MAGE | MSSEQKSQHCKPEEGVEAQEEALGLVGAQAPTT | Core Human ORF Only |
| | 1014 | RFS | | A4 | EEQEAAVSSSSPLVPGTLEEVPAAESAGPPQSPQ | |
| | | | | | GASALPTTISFTCWRQPNEGSSSQEEEGPSTSPD | |
| | | | | | AESLFREALSNKVDELAHFLLRKYRAKELVTKAE | |
| | | | | | MLERVIKNYKRCFPVIFGKASESLKMIFGIDVKEVD | |
| | | | | | PTSNTYTLVTCLGLSYDGLLGNNQIFPKTGLLIIVL | |
| | | | | | GTIAMEGDSASEEEIWEELGVMGVYDGREHTVY | |
| | | | | | GEPRKLLTQDVWQENYLEYRQVPGSNPARYEFL | |
| | | | | | WGPRALAETSYVKVLEHVVRVNARVRIAYPSLRE | |
| | | | | | AALLEEEEGV | |
| 11 | JHUO | IPL | 0.493293692 | SLC43 | MSPILGYWKIKGLVQPTRLLLEYLEEKYEEHLYER | Full Literal Sequence- |
| | 1711 | RFS | | A2 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAPTLATAHRRRWW | |
| | | | | | MACTAVLENLLFSAVLLGWGSLLIMLKSEGFYSYL | |
| | | | | | CTEPENVTNGTVGGTAEPGHEEVSWMNGWLSC | |
| | | | | | QAQDEMLNLAFTVGSFLLSAITLPLGIVMDKYGPR | |
| | | | | | KLRLLGSACFAVSCLLIAYGASKPNALSVLIFIALAL | |
| | | | | | NGFGGMCMTFTSLTLPNMFGDLRSTFIALMIGSY | |
| | | | | | ASSAVTFPGIKLIYDAGVSFIVVLVVWAGCSGLVF | |
| | | | | | LNCFFNWPLEPFPGPEDMDYSVKIKFSWLGFDH | |
| | | | | | KITGKQFYKQVTTVGRRLSVGSSMRSAKEQVALQ | |
| | | | | | EGHKLCLSTVDLEVKCQPDAAVAPSFMHSVFSPI | |
| | | | | | LLLSLVTMCVTQLRLIFYMGAMNNILKFLVSGDQK | |
| | | | | | TVGLYTSIFGVLQLLCLLTAPVIGYIMDWRLKECE | |
| | | | | | DASEEPEEKDANQGEKKKKKRDRQIQKITNAMRAFA | |
| | | | | | FTNLLLVGFGVTCLIPNLPLQILSFILHTIVRGFIH | |
| | | | | | SAVGGLYAAVYPSTQFGSLTGLQSLISALFALLQQ | |
| | | | | | PLFLAMMGPLQGDPLVWNVGLLLLSLLGFCLPLY | |
| | | | | | LICYRRQLERQLQQRQEDDKLFLKINGSSNQEAF | |
| | | | | | V | |
| 12 | JHUO | IPL | 0.493293692 | SLC43 | MAPTLATAHRRRVWVMACTAVLENLLFSAVLLGW | Core Human ORF Only |
| | 1711 | RFS | | A2 | GSLLIMLKSEGFYSYLCTEPENVTNGTVGGTAEP | |
| | | | | | GHEEVSWMNGWLSCQAQDEMLNLAFTVGSFLL | |
| | | | | | SAITLPLGIVMDKYGPRKLRLLGSACFAVSCLLIAY | |
| | | | | | GASKPNALSVLIFIALALNGFGGMCMTFTSLTLPN | |
| | | | | | MFGDLRSTFIALMIGSYASSAVTFPGIKLIYDAGVS | |
| | | | | | FIVVLVVWAGCSGLVFLNCFFNWPLEPFPGPEDM | |
| | | | | | DYSVKIKFSWLGFDHKITGKQFYKQVTTVGRRLS | |
| | | | | | VGSSMRSAKEQVALQEGHKLCLSTVDLEVKCQP | |
| | | | | | DAAVAPSFMHSVFSPILLLSLVTMCVTQLRLIFYM | |
| | | | | | GAMNNILKFLVSGDQKTVGLYTSIFGVLQLLCLLT | |
| | | | | | APVIGYIMDWRLKECEDASEEPEEKDANQGEKKK | |
| | | | | | KKRDRQIQKITNAMRAFAFTNLLLVGFGVTCLIPN | |
| | | | | | LPLQILSFILHTIVRGFIHSAVGGLYAAVYPSTQFG | |
| | | | | | SLTGLQSLISALFALLQQPLFLAMMGPLQGDPLW | |
| | | | | | VNVGLLLLSLLGFCLPLYLICYRRQLERQLQQRQE | |
| | | | | | DDKLFLKINGSSNQEAFV | |
| 13 | JHUO | IPL | 0.075427692 | CELA3 | MSPILGYWKIKGLVQPTRLLLEYLEEKYEEHLYER | Full Literal Sequence- |
| | 1950 | RFS | | B | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMLRLLSSLLLVAVA | |
| | | | | | SGYGPPSSRPSSRVVNGEDAVPYSWPWQVSLQ | |
| | | | | | YEKSGSFYHTCGGSLIAPDVWVTAGHCISSSWTY | |
| | | | | | QVVLGEYDRAVKEGPEQVIPINSGDLFVHPLWNR | |
| | | | | | SCVACGNDIALIKLSRSAQLGDAVQLASLPPAGDI | |
| | | | | | LPNETPCYITGWGRLYTNGPLPDKLQEALLPVVD | |
| | | | | | YEHCSRWNWWGSSVKKTMVCAGGDIRSGCNG | |
| | | | | | DSGGPLNCPTEDGGWQVHGVTSFVSAFGCNTR | |
| | | | | | RKPTVFTRVSAFIDWIEETIASH | |
| 14 | JHUO | IPI_ | 0.075427692 | CELA3 | MMLRLLSSLLLVAVASGYGPPSSRPSSRVVNGED | Core Human ORF Only |
| | 1950 | RFS | | B | AVPYSWPWQVSLQYEKSGSFYHTCGGSLIAPDW | |
| | | | | | VVTAGHCISSSWTYQVVLGEYDRAVKEGPEQVIP | |
| | | | | | INSGDLFVHPLWNRSCVACGNDIALIKLSRSAQLG | |
| | | | | | DAVQLASLPPAGDILPNETPCYITGWGRLYTNGP | |
| | | | | | LPDKLQEALLPVVDYEHCSRWNVWVGSSVKKTM | |
| | | | | | VCAGGDIRSGCNGDSGGPLNCPTEDGGWQVHG | |
| | | | | | VTSFVSAFGCNTRRKPTVFTRVSAFIDWIEETIAS | |
| | | | | | H | |
| 15 | JHUO | IPI_ | 0.253778639 | MAGE | MSPILGYWKIKGLVQPTRLLLEYLEEKYEEHLYER | Full Literal Sequence- |
| | 2306 | RFS | | A11 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPLEQRSQHCKPEE | |
| | | | | | GLQAQEEDLGLVGAQALQAEEQEAAFFSSTLNV | |
| | | | | | GTLEELPAAESPSPPQSPQEESFSPTAMDAIFGS | |
| | | | | | LSDEGSGSQEKEGPSTSPDLIDPESFSQDILHDKII | |
| | | | | | DLVHLLLRKYRVKGLITKAEMLGSVIKNYEDYFPEI | |
| | | | | | FREASVCMQLLFGIDVKEVDPTSHSYVLVTSLNLS | |
| | | | | | YDGIQCNEQSMPKSGLLIIVLGVIFMEGNCIPEEV | |
| | | | | | MWEVLSIMGVYAGREHFLFGEPKRLLTQNWVQE | |
| | | | | | KYLVYRQVPGTDPACYEFLWGPRAHAETSKMKV | |
| | | | | | LEYIANANGRDPTSYPSLYEDALREEGEGV | |
| 16 | JHUO | IPL | 0.253778639 | MAGE | MPLEQRSQHCKPEEGLQAQEEDLGLVGAQALQA | Core Human ORF Only |
| | 2306 | RFS | | A11 | EEQEAAFFSSTLNVGTLEELPAAESPSPPQSPQE | |
| | | | | | ESFSPTAMDAIFGSLSDEGSGSQEKEGPSTSPDL | |
| | | | | | IDPESFSQDILHDKIIDLVHLLLRKYRVKGLITKAEM | |
| | | | | | LGSVIKNYEDYFPEIFREASVCMQLLFGIDVKEVD | |
| | | | | | PTSHSYVLVTSLNLSYDGIQCNEQSMPKSGLLIIVL | |
| | | | | | GVIFMEGNCIPEEVMWEVLSIMGVYAGREHFLFG | |
| | | | | | EPKRLLTQNWVQEKYLVYRQVPGTDPACYEFLW | |
| | | | | | GPRAHAETSKMKVLEYIANANGRDPTSYPSLYED | |
| | | | | | ALREEGEGV | |
| 17 | JHUO | IPL | 0.172297072 | ECSIT | MSPILGYWKIKGLVQPTRLLLEYLEEKYEEHLYER | Full Literal Sequence- |
| | 2435 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSWVQATLLARGLC | |
| | | | | | RAWGGTCGAALTGTSISQVPRRLPRGLHCSAAA | |
| | | | | | HSSEQSLVPSPPEPRQRPTKALVPFEDLFGQAPG | |
| | | | | | GERDKASFLQTVQKFAEHSVRKRGHIDFIYLALRK | |
| | | | | | MREYGVERDLAVYNQLLNIFPKEVFRPRNIIQRIFV | |
| | | | | | HYPRQQECGIAVLEQMENHGVMPNKETEFLLIQI | |
| | | | | | FGRKSYPMLKLVRLKLWFPRFMNVNPFPVPRDL | |
| | | | | | PQDPVELAMFGLRHMEPDLSARVTIYQVPLPKDS | |
| | | | | | TGAADPPQPHIVGIQSPDQQAALARHNPARPVFV | |
| | | | | | EGPFSLWLRNKCVYYHILRADLLPPEEREVEETP | |
| | | | | | EEWNLYYPMQLDLEYVRSGWDNYEFDINEVEEG | |
| | | | | | PVFAMCMAGAHDQATMAKWIQGLQETNPTLAQI | |
| | | | | | PVVFRLAGSTRELQTSSAGLEEPPLPEDHQEEDD | |
| | | | | | NLQRQQQGQS | |
| 18 | JHUO | IPL | 0.172297072 | ECSIT | MSVWQATLLARGLCRAWGGTCGAALTGTSISQV | Core Human ORF Only |
| | 2435 | RFS | | | PRRLPRGLHCSAAAHSSEQSLVPSPPEPRQRPT | |
| | | | | | KALVPFEDLFGQAPGGERDKASFLQTVQKFAEHS | |
| | | | | | VRKRGHIDFIYLALRKMREYGVERDLAVYNQLLNI | |
| | | | | | FPKEVFRPRNIIQRIFVHYPRQQECGIAVLEQMEN | |
| | | | | | HGVMPNKETEFLLIQIFGRKSYPMLKLVRLKLWFP | |
| | | | | | RFMNVNPFPVPRDLPQDPVELAMFGLRHMEPDL | |
| | | | | | SARVTIYQVPLPKDSTGAADPPQPHIVGIQSPDQ | |
| | | | | | QAALARHNPARPVFVEGPFSLWLRNKCVYYHILR | |
| | | | | | ADLLPPEEREVEETPEEWNLYYPMQLDLEYVRS | |
| | | | | | GWDNYEFDINEVEEGPVFAMCMAGAHDQATMA | |
| | | | | | KWIQGLQETNPTLAQIPVVFRLAGSTRELQTSSA | |
| | | | | | GLEEPPLPEDHQEEDDNLQRQQQGQS | |
| 19 | JHUO | IPL | 0.073552721 | C7orf25 | MSPILGYWKIKGLVQPTRLLLEYLEEKYEEHLYER | Full Literal Sequence- |
| | 3477 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSAHSMLCERIAIAK | |
| | | | | | ELIKRAESLSRSRKGGIEGGAKLCSKLKAELKFLQ | |
| | | | | | KVEAGKVAIKESHLQSTNLTHLRAIVESAENLEEV | |
| | | | | | VSVLHVFGYTDTLGEKQTLVVDVVANGGHTWVK | |
| | | | | | AIGRKAEALHNIWLGRGQYGDKSIIEQAEDFLQAS | |
| | | | | | HQQPVQYSNPHIIFAFYNSVSSPMAEKLKEMGISV | |
| | | | | | RGDIVAVNALLDHPEELQPSESESDDEGPELLQV | |
| | | | | | TRVDRENILASVAFPTEIKVDVCKRVNLDITTLITYV | |
| | | | | | SALSYGGCHFIFKEKVLTEQAEQERKEQVLPQLE | |
| | | | | | AFMKDKELFACESAVKDFQSILDTLGGPGERERA | |
| | | | | | TVLIKRINVVPDQPSERALRLVASSKINSRSLTIFG | |
| | | | | | TGDTLKAITMTANSGFVRAANNQGVKFSVFIHQP | |
| | | | | | RALTESKEALATPLPKDYTTDSEH | |
| 20 | JHUO | IPL | 0.073552721 | C7orf25 | MSAHSMLCERIAIAKELIKRAESLSRSRKGGIEGG | Core Human ORF Only |
| | 3477 | RFS | | | AKLCSKLKAELKFLQKVEAGKVAIKESHLQSTNLT | |
| | | | | | HLRAIVESAENLEEVVSVLHVFGYTDTLGEKQTLV | |
| | | | | | VDVVANGGHTWVKAIGRKAEALHNIWLGRGQYG | |
| | | | | | DKSIIEQAEDFLQASHQQPVQYSNPHIIFAFYNSV | |
| | | | | | SSPMAEKLKEMGISVRGDIVAVNALLDHPEELQP | |
| | | | | | SESESDDEGPELLQVTRVDRENILASVAFPTEIKV | |
| | | | | | DVCKRVNLDITTLITYVSALSYGGCHFIFKEKVLTE | |
| | | | | | QAEQERKEQVLPQLEAFMKDKELFACESAVKDF | |
| | | | | | QSILDTLGGPGERERATVLIKRINVVPDQPSERAL | |
| | | | | | RLVASSKINSRSLTIFGTGDTLKAITMTANSGFVRA | |
| | | | | | ANNQGVKFSVFIHQPRALTESKEALATPLPKDYTT | |
| | | | | | DSEH | |
| 21 | JHUO | IPL | 0.116480939 | MYOG | MSPILGYWKIKGLVQPTRLLLEYLEEKYEEHLYER | Full Literal Sequence- |
| | 3708 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMELYETSPYFYQEPR | |
| | | | | | FYDGENYLPVHLQGFEPPGYERTELTLSPEAPGP | |
| | | | | | LEDKGLGTPEHCPGQCLPWACKVCKRKSVSVDR | |
| | | | | | RRAATLREKRRLKKVNEAFEALKRSTLLNPNQRL | |
| | | | | | PKVEILRSAIQYIERLQALLSSLNQEERDLRYRGG | |
| | | | | | GGPQPGVPSECSSHSASCSPEWGSALEFSANP | |
| | | | | | GDHLLTADPTDAHNLHSLTSIVDSITVEDVSVAFP | |
| | | | | | DETMPN | |
| 22 | JHUO | IPL | 0.116480939 | MYOG | MELYETSPYFYQEPRFYDGENYLPVHLQGFEPP | Core Human ORF Only |
| | 3708 | RFS | | | GYERTELTLSPEAPGPLEDKGLGTPEHCPGQCLP | |
| | | | | | WACKVCKRKSVSVDRRRAATLREKRRLKKVNEA | |
| | | | | | FEALKRSTLLNPNQRLPKVEILRSAIQYIERLQALL | |
| | | | | | SSLNQEERDLRYRGGGGPQPGVPSECSSHSAS | |
| | | | | | CSPEWGSALEFSANPGDHLLTADPTDAHNLHSLT | |
| | | | | | SIVDSITVEDVSVAFPDETMPN | |
| 23 | JHUO | IPL | 0.725053022 | GNPAT | MSPILGYWKIKGLVQPTRLLLEYLEEKYEEHLYER | Full Literal Sequence- |
| | 3972 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMESSSSSNSYFSVG | |
| | | | | | PTSPSAVVLLYSKELKKWDEFEDILEERRHVSDLK | |
| | | | | | FAMKCYTPLVYKGITPCKPIDIKCSVLNSEEIHYVIK | |
| | | | | | QLSKESLQSVDVLREEVSEILDEMSHKLRLGAIRF | |
| | | | | | CAFTLSKVFKQIFSKVCVNEEGIQKLQRAIQEHPV | |
| | | | | | VLLPSHRSYIDFLMLSFLLYNYDLPVPVIAAGMDFL | |
| | | | | | GMKMVGELLRMSGAFFMRRTFGGNKLYWAVFS | |
| | | | | | EYVKTMLRNGYAPVEFFLEGTRSRSAKTLTPKFG | |
| | | | | | LLNIVMEPFFKREVFDTYLVPISISYDKILEETLYVY | |
| | | | | | ELLGVPKPKESTTGLLKARKILSENFGSIHVYFGD | |
| | | | | | PVSLRSLAAGRMSRSSYNLVPRYIPQKQSEDMH | |
| | | | | | AFVTEVAYKMELLQIENMVLSPWTLIVAVLLQNRP | |
| | | | | | SMDFDALVEKTLWLKGLTQAFGGFLIWPDNKPAE | |
| | | | | | EVVPASILLHSNIASLVKDQVILKVDSGDSEVVDGL | |
| | | | | | MLQHITLLMCSAYRNQLLNIFVRPSLVAVALQMTP | |
| | | | | | GFRKEDVYSCFRFLRDVFADEFIFLPGNTLKDFEE | |
| | | | | | GCYLLCKSEAIQVTTKDILVTEKGNTVLEFLVGLFK | |
| | | | | | PFVESYQIICKHLLSEEEDHFSEEQYLAAVRKFTS | |
| | | | | | QLLDQGTSQCYDVLSSDVQKNALAACVRLGVVE | |
| | | | | | KKKINNNCIFNVNEPATTKLEEMLGCKTPIGKPAT | |
| | | | | | AKL | |
| 24 | JHUO | IPL | 0.725053022 | GNPAT | MESSSSSNSYFSVGPTSPSAVVLLYSKELKKWDE | Core Human ORF Only |
| | 3972 | RFS | | | FEDILEERRHVSDLKFAMKCYTPLVYKGITPCKPI | |
| | | | | | DIKCSVLNSEEIHYVIKQLSKESLQSVDVLREEVSE | |
| | | | | | ILDEMSHKLRLGAIRFCAFTLSKVFKQIFSKVCVNE | |
| | | | | | EGIQKLQRAIQEHPVVLLPSHRSYIDFLMLSFLLYN | |
| | | | | | YDLPVPVIAAGMDFLGMKMVGELLRMSGAFFMR | |
| | | | | | RTFGGNKLYWAVFSEYVKTMLRNGYAPVEFFLE | |
| | | | | | GTRSRSAKTLTPKFGLLNIVMEPFFKREVFDTYLV | |
| | | | | | PISISYDKILEETLYVYELLGVPKPKESTTGLLKAR | |
| | | | | | KILSENFGSIHVYFGDPVSLRSLAAGRMSRSSYNL | |
| | | | | | VPRYIPQKQSEDMHAFVTEVAYKMELLQIENMVL | |
| | | | | | SPWTLIVAVLLQNRPSMDFDALVEKTLWLKGLTQ | |
| | | | | | AFGGFLIWPDNKPAEEVVPASILLHSNIASLVKDQ | |
| | | | | | VILKVDSGDSEVVDGLMLQHITLLMCSAYRNQLLN | |
| | | | | | IFVRPSLVAVALQMTPGFRKEDVYSCFRFLRDVF | |
| | | | | | ADEFIFLPGNTLKDFEEGCYLLCKSEAIQVTTKDIL | |
| | | | | | VTEKGNTVLEFLVGLFKPFVESYQIICKHLLSEEED | |
| | | | | | HFSEEQYLAAVRKFTSQLLDQGTSQCYDVLSSDV | |
| | | | | | QKNALAACVRLGVVEKKKINNNCIFNVNEPATTKL | |
| | | | | | EEMLGCKTPIGKPATAKL | |
| 25 | JHUO | IPL | 0.165164106 | SELPL | MSPILGYWKIKGLVQPTRLLLEYLEEKYEEHLYER | Full Literal Sequence- |
| | 4013 | RFS | | G | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPLQLLLLLILLGPGN | |
| | | | | | SLQLWDTWADEAEKALGPLLARDRRQATEYEYL | |
| | | | | | DYDFLPETEPPEMLRNSTDTTPLTGPGTPESTTV | |
| | | | | | EPAARRSTGLDAGGAVTELTTELANMGNLSTDSA | |
| | | | | | AMEIQTTQPAATEAQTTPLAATEAQTTRLTATEAQ | |
| | | | | | TTPLAATEAQTTPPAATEAQTTQPTGLEAQTTAP | |
| | | | | | AAMEAQTTAPAAMEAQTTPPAAMEAQTTQTTAM | |
| | | | | | EAQTTAPEATEAQTTQPTATEAQTTPLAAMEALS | |
| | | | | | TEPSATEALSMEPTTKRGLFIPFSVSSVTHKGIPM | |
| | | | | | AASNLSVNYPVGAPDHISVKQCLLAILILALVATIFF | |
| | | | | | VCTVVLAVRLSRKGHMYPVRNYSPTEMVCISSLL | |
| | | | | | PDGGEGPSATANGGLSKAKSPGLTPEPREDREG | |
| | | | | | DDLTLHSFLP | |
| 26 | JHUO | IPL | 0.165164106 | SELPL | MPLQLLLLLILLGPGNSLQLWDTWADEAEKALGP | Core Human ORF Only |
| | 4013 | RFS | | G | LLARDRRQATEYEYLDYDFLPETEPPEMLRNSTD | |
| | | | | | TTPLTGPGTPESTTVEPAARRSTGLDAGGAVTEL | |
| | | | | | TTELANMGNLSTDSAAMEIQTTQPAATEAQTTPL | |
| | | | | | AATEAQTTRLTATEAQTTPLAATEAQTTPPAATEA | |
| | | | | | QTTQPTGLEAQTTAPAAMEAQTTAPAAMEAQTT | |
| | | | | | PPAAMEAQTTQTTAMEAQTTAPEATEAQTTQPT | |
| | | | | | ATEAQTTPLAAMEALSTEPSATEALSMEPTTKRG | |
| | | | | | LFIPFSVSSVTHKGIPMAASNLSVNYPVGAPDHIS | |
| | | | | | VKQCLLAILILALVATIFFVCTVVLAVRLSRKGHMY | |
| | | | | | PVRNYSPTEMVCISSLLPDGGEGPSATANGGLSK | |
| | | | | | AKSPGLTPEPREDREGDDLTLHSFLP | |
| 27 | JHUO | IPL | 0.325184572 | SPACA | MSPILGYWKIKGLVQPTRLLLEYLEEKYEEHLYER | Full Literal Sequence- |
| | 4208 | RFS | | 1 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSPRGTGCSAGLLM | |
| | | | | | TVGWLLLAGLQSARGTNVTAAVQDAGLAHEGEG | |
| | | | | | EEETENNDSETAENYAPPETEDVSNRNVVKEVEF | |
| | | | | | GMCTVTCGIGVREVILTNGCPGGESKCVVRVEEC | |
| | | | | | RGPTDCGWGKPISESLESVRLACIHTSPLNRFKY | |
| | | | | | MWKLLRQDQQSIILVNDSAILEVRKESHPLAFECD | |
| | | | | | TLDNNEIVATIKFTVYTSSELQMRRSSLPATDAALI | |
| | | | | | FVLTIGVIICVFIIFLLIFIIINWAAVKAFWGAKAS | |
| | | | | | TPEVQSEQSSVRYKDSTSLDQLPTEMPGEDDALSEW | |
| | | | | | NE | |
| 28 | JHUO | IPI_ | 0.325184572 | SPACA | MSPRGTGCSAGLLMTVGWLLLAGLQSARGTNVT | Core Human ORF Only |
| | 4208 | RFS | | 1 | AAVQDAGLAHEGEGEEETENNDSETAENYAPPE | |
| | | | | | TEDVSNRNVVKEVEFGMCTVTCGIGVREVILTNG | |
| | | | | | CPGGESKCVVRVEECRGPTDCGWGKPISESLES | |
| | | | | | VRLACIHTSPLNRFKYMWKLLRQDQQSIILVNDSA | |
| | | | | | ILEVRKESHPLAFECDTLDNNEIVATIKFTVYTSSE | |
| | | | | | LQMRRSSLPATDAALIFVLTIGVIICVFIIFLLIFI | |
| | | | | | IINWAAVKAFWGAKASTPEVQSEQSSVRYKDSTSLDQ | |
| | | | | | LPTEMPGEDDALSEWNE | |
| 29 | JHUO | IPI_ | 0.207262175 | BNIPL | MSPILGYWKIKGLVQPTRLLLEYLEEKYEEHLYER | Full Literal Sequence- |
| | 4327 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMRKRLSAPELRLSLT | |
| | | | | | KGPGNDGASPTQSAPSSPDGSSDLEIDELETPSD | |
| | | | | | SEQLDSGHEFEWEDELPRAEGLGTSETAERLGR | |
| | | | | | GCMWDVTGEDGHHWRVFRMGPREQRVDMTVIE | |
| | | | | | PYKKVLSHGGYHGDGLNAVILFASCYLPRSSIPNY | |
| | | | | | TYVMEHLFRYMVGTLELLVAENYLLVHLSGGTSR | |
| | | | | | AQVPPLSWIRQCYRTLDRRLRKNLRALVVVHATWY | |
| | | | | | VKAFLALLRPFISSKFTRKIRFLDSLGELAQLISLD | |
| | | | | | QVHIPEAVRQLDRDLHGSGGT | |
| 30 | JHUO | IPL | 0.207262175 | BNIPL | MRKRLSAPELRLSLTKGPGNDGASPTQSAPSSP | Core Human ORF Only |
| | 4327 | RFS | | | DGSSDLEIDELETPSDSEQLDSGHEFEWEDELPR | |
| | | | | | AEGLGTSETAERLGRGCMWDVTGEDGHHWRVF | |
| | | | | | RMGPREQRVDMTVIEPYKKVLSHGGYHGDGLNA | |
| | | | | | VILFASCYLPRSSIPNYTYVMEHLFRYMVGTLELL | |
| | | | | | VAENYLLVHLSGGTSRAQVPPLSWIRQCYRTLDR | |
| | | | | | RLRKNLRALVVVHATWYVKAFLALLRPFISSKFTR | |
| | | | | | KIRFLDSLGELAQLISLDQVHIPEAVRQLDRDLHG | |
| | | | | | SGGT | |
| 31 | JHUO | IPL | 0.103309294 | MFSD9 | MSPILGYWKIKGLVQPTRLLLEYLEEKYEEHLYER | Full Literal Sequence- |
| | 4653 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMELGGHWDMNSAP | |
| | | | | | RLVSETAERKQEQKTGTEAEAADSGAVGARRFLL | |
| | | | | | CLYLVGFLDLFGVSMVVPLLSLHVKSLGASPTVA | |
| | | | | | GIVGSSYGILQLFSSTLVGCWSDVVGRRSSLLACI | |
| | | | | | LLSALGYLLLGAATNVFLFVLARVPAGIFKHTLSIS | |
| | | | | | RALLSDVVPEKERPLVIGHFNTASGVGFILGPVVG | |
| | | | | | GYLTELEDGFYLTAFICFLVFILNAGLVWFFPWRE | |
| | | | | | AKPGSTEKGLPLRKTHVLLGRSHDTVQEAATSRR | |
| | | | | | ARASKKTAQPWVEVVLALRNMKNLLFSEMWDIFL | |
| | | | | | VRLLMAMAVMLYYSNFVLALEERFGVRPKVTGYL | |
| | | | | | ISYSSMLGAVAGLALGPILRLYKHNSQALLLHSSIL | |
| | | | | | TCTLLLLYSLAPTMGAVVLSSTLLSFSTAIGRTCIT | |
| | | | | | DLQLTVGGAQASGTLIGVGQSVTAVGRIIAPLLSG | |
| | | | | | VAQEVSPCGPPSLGAVLALVAIFIMSLNKRHSSGD | |
| | | | | | GNSKLKSE | |
| 32 | JHUO | IPL | 0.103309294 | MFSD9 | MELGGHWDMNSAPRLVSETAERKQEQKTGTEA | Core Human ORF Only |
| | 4653 | RFS | | | EAADSGAVGARRFLLCLYLVGFLDLFGVSMVVPL | |
| | | | | | LSLHVKSLGASPTVAGIVGSSYGILQLFSSTLVGC | |
| | | | | | WSDVVGRRSSLLACILLSALGYLLLGAATNVFLFV | |
| | | | | | LARVPAGIFKHTLSISRALLSDVVPEKERPLVIGHF | |
| | | | | | NTASGVGFILGPVVGGYLTELEDGFYLTAFICFLV | |
| | | | | | FILNAGLVWFFPWREAKPGSTEKGLPLRKTHVLL | |
| | | | | | GRSHDTVQEAATSRRARASKKTAQPWVEVVLAL | |
| | | | | | RNMKNLLFSEMWDIFLVRLLMAMAVMLYYSNFVL | |
| | | | | | ALEERFGVRPKVTGYLISYSSMLGAVAGLALGPIL | |
| | | | | | RLYKHNSQALLLHSSILTCTLLLLYSLAPTMGAVVL | |
| | | | | | SSTLLSFSTAIGRTCITDLQLTVGGAQASGTLIGVG | |
| | | | | | QSVTAVGRIIAPLLSGVAQEVSPCGPPSLGAVLAL | |
| | | | | | VAIFIMSLNKRHSSGDGNSKLKSE | |
| 33 | JHUO | IPL | 0.404266893 | ZFYVE | MSPILGYWKIKGLVQPTRLLLEYLEEKYEEHLYER | Full Literal Sequence- |
| | 4797 | RFS | | 19 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMESRCYGCAVKFTL | |
| | | | | | FKKEYGCKNCGRAFCSGCLSFSAAVPRTGNTQQ | |
| | | | | | KVCKQCHEVLTRGSSANASKWSPPQNYKKRVAA | |
| | | | | | LEAKQKPSTSQSQGLTRQDQMIAERLARLRQEN | |
| | | | | | KPKLVPSQAEIEARLAALKDERQGSIPSTQEMEA | |
| | | | | | RLAALQGRVLPSQTPQPAHHTPDTRTQAQQTQD | |
| | | | | | LLTQLAAEVAIDESWKGGGPAASLQNDLNQGGP | |
| | | | | | GSTNSKRQANWSLEEEKSRLLAEAALELREENTR | |
| | | | | | QERILALAKRLAMLRGQDPERVTLQDYRLPDSDD | |
| | | | | | DEDEETAIQRVLQQLTEEAALDEASGFNIPAEQAS | |
| | | | | | RPWTQPRGAEPEAQDVDPRPEAEEEELPWCCIC | |
| | | | | | NEDATLRCAGCDGDLFCARCFREGHDAFELKEH | |
| | | | | | QTSAYSPPRAGQEH | |
| 34 | JHUO | IPL | 0.404266893 | ZFYVE | MESRCYGCAVKFTLFKKEYGCKNCGRAFCSGCL | Core Human ORF Only |
| | 4797 | RFS | | 19 | SFSAAVPRTGNTQQKVCKQCHEVLTRGSSANAS | |
| | | | | | KWSPPQNYKKRVAALEAKQKPSTSQSQGLTRQD | |
| | | | | | QMIAERLARLRQENKPKLVPSQAEIEARLAALKDE | |
| | | | | | RQGSIPSTQEMEARLAALQGRVLPSQTPQPAHH | |
| | | | | | TPDTRTQAQQTQDLLTQLAAEVAIDESWKGGGP | |
| | | | | | AASLQNDLNQGGPGSTNSKRQANWSLEEEKSRL | |
| | | | | | LAEAALELREENTRQERILALAKRLAMLRGQDPE | |
| | | | | | RVTLQDYRLPDSDDDEDEETAIQRVLQQLTEEAA | |
| | | | | | LDEASGFNIPAEQASRPWTQPRGAEPEAQDVDP | |
| | | | | | RPEAEEEELPWCCICNEDATLRCAGCDGDLFCA | |
| | | | | | RCFREGHDAFELKEHQTSAYSPPRAGQEH | |
| 35 | JHUO | IPL | 0.127679716 | SESN3 | MSPILGYWKIKGLVQPTRLLLEYLEEKYEEHLYER | Full Literal Sequence- |
| | 4972 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNRGGGSPSAAANY | |
| | | | | | LLCTNCRKVLRKDKRIRVSQPLTRGPSAFIPEKEV | |
| | | | | | VQANTVDERTNFLVEEYSTSGRLDNITQVMSLHT | |
| | | | | | QYLESFLRSQFYMLRMDGPLPLPYRHYIAIMAAA | |
| | | | | | RHQCSYLINMHVDEFLKTGGIAEWLNGLEYVPQR | |
| | | | | | LKNLNEINKLLAHRPWLITKEHIQKLVKTGENNWS | |
| | | | | | LPELVHAVVLLAHYHALASFVFGSGINPERDPEIS | |
| | | | | | NGFRLISVNNFCVCDLANDNNIENASLSGSNFGIV | |
| | | | | | DSLSELEALMERMKRLQEEREDEEASQEEMSTR | |
| | | | | | FEKEKKESLFVVSGDTFHSFPHSGAFLHFFAF | |
| 36 | JHUO | IPL | 0.127679716 | SESN3 | MNRGGGSPSAAANYLLCTNCRKVLRKDKRIRVS | Core Human ORF Only |
| | 4972 | RFS | | | QPLTRGPSAFIPEKEVVQANTVDERTNFLVEEYS | |
| | | | | | TSGRLDNITQVMSLHTQYLESFLRSQFYMLRMDG | |
| | | | | | PLPLPYRHYIAIMAAARHQCSYLINMHVDEFLKTG | |
| | | | | | GIAEWLNGLEYVPQRLKNLNEINKLLAHRPWLITK | |
| | | | | | EHIQKLVKTGENNWSLPELVHAVVLLAHYHALAS | |
| | | | | | FVFGSGINPERDPEISNGFRLISVNNFCVCDLAND | |
| | | | | | NNIENASLSGSNFGIVDSLSELEALMERMKRLQE | |
| | | | | | EREDEEASQEEMSTRFEKEKKESLFVVSGDTFHS | |
| | | | | | FPHSGAFLHFFAF | |
| 37 | JHUO | IPL | 0.109312832 | CALHM | MSPILGYWKIKGLVQPTRLLLEYLEEKYEEHLYER | Full Literal Sequence- |
| | 5595 | RFS | | 1 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMDKFRMIFQFLQS | |
| | | | | | NQESFMNGICGIMALASAQMYSAFDFNCPCLPGY | |
| | | | | | NAAYSAGILLAPPLVLFLLGLVMNNNVSMLAEEW | |
| | | | | | KRPPGRRAKDPAVLRYMFCSMAQRALIAPVVVWV | |
| | | | | | AVTLLDGKCFLCAFCTAVPVSALGNGSLAPGLPA | |
| | | | | | PELARLLARVPCPEIYDGDWLLAREVAVRYLRCIS | |
| | | | | | QALGWSFVLLTTLLAFVVRSVRPCFTQAAFLKSK | |
| | | | | | YWSHYIDIERKLFDETCTEHAKAFAKVCIQQFFEA | |
| | | | | | MNHDLELGHTNGTLATAPASAAAPTTPDGAEEER | |
| | | | | | EKLRGITDQGTMNRLLTSWHKCKPPLRLGQEEPP | |
| | | | | | LMGNGWAGGGPRPPRKEVATYFSKV | |
| 38 | JHUO | IPL | 0.109312832 | CALHM | MMDKFRMIFQFLQSNQESFMNGICGIMALASAQ | Core Human ORF Only |
| | 5595 | RFS | | 1 | MYSAFDFNCPCLPGYNAAYSAGILLAPPLVLFLLG | |
| | | | | | LVMNNNVSMLAEEWKRPPGRRAKDPAVLRYMF | |
| | | | | | CSMAQRALIAPVVVWVAVTLLDGKCFLCAFCTAVP | |
| | | | | | VSALGNGSLAPGLPAPELARLLARVPCPEIYDGD | |
| | | | | | WLLAREVAVRYLRCISQALGWSFVLLTTLLAFVVR | |
| | | | | | SVRPCFTQAAFLKSKYWSHYIDIERKLFDETCTEH | |
| | | | | | AKAFAKVCIQQFFEAMNHDLELGHTNGTLATAPA | |
| | | | | | SAAAPTTPDGAEEEREKLRGITDQGTMNRLLTSW | |
| | | | | | HKCKPPLRLGQEEPPLMGNGWAGGGPRPPRKE | |
| | | | | | VATYFSKV | |
| 39 | JHUO | IPL | 0.252688604 | DAD1 | MSPILGYWKIKGLVQPTRLLLEYLEEKYEEHLYER | Full Literal Sequence- |
| | 6252 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSASVVSVISRFLEE | |
| | | | | | YLSSIPQRLKLLDAYLLYILLTGALQFGYCLLVGTF | |
| | | | | | PFNSFLSGFISCVGSFILAVCLRIQINPQNKADFQG | |
| | | | | | ISPERAFADFLFASTILHLVVMNFVG | |
| 40 | JHUO | IPL | 0.252688604 | DAD1 | MSASVVSVISRFLEEYLSSIPQRLKLLDAYLLYILLT | Core Human ORF Only |
| | 6252 | RFS | | | GALQFGYCLLVGTFPFNSFLSGFISCVGSFILAVC | |
| | | | | | LRIQINPQNKADFQGISPERAFADFLFASTILHLVV | |
| | | | | | MNFVG | |
| 41 | JHUO | IPL | 0.278591153 | UFSP2 | MSPILGYWKIKGLVQPTRLLLEYLEEKYEEHLYER | Full Literal Sequence- |
| | 6349 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVISESMDILFRIRGG | |
| | | | | | LDLAFQLATPNEIFLKKALKHVLSDLSTKLSSNALV | |
| | | | | | FRICHSSVYIWPSSDINTIPGELTDASACKTILRFIQ | |
| | | | | | FEPEEDIKRKFMRKKDKKLSDMHQIVNIDLMLEMS | |
| | | | | | TSLAAVTPIIERESGGHHYVNMTLPVDAVISVAPE | |
| | | | | | ETWGKVRKLLVDAIHNQLTDMEKCILKYMKGTSIV | |
| | | | | | VPEPLHFLLPGKKNLVTISYPSGIPDGQLQAYRKE | |
| | | | | | LHDLFNLPHDRPYFKRSNAYHFPDEPYKDGYIRN | |
| | | | | | PHTYLNPPNMETGMIYVVQGIYGYHHYMQDRIDD | |
| | | | | | NGWGCAYRSLQTICSWFKHQGYTERSIPTHREIQ | |
| | | | | | QALVDAGDKPATFVGSRQWIGSIEVQLVLNQLIGI | |
| | | | | | TSKILFVSQGSEIASQGRELANHFQSEGTPVMIGG | |
| | | | | | GVLAHTILGVAWNEITGQIKFLILDPHYTGAEDLQV | |
| | | | | | ILEKGWCGWKGPDFWNKDAYYNLCLPQRPNMI | |
| 42 | JHUO | IPL | 0.278591153 | UFSP2 | MVISESMDILFRIRGGLDLAFQLATPNEIFLKKALK | Core Human ORF Only |
| | 6349 | RFS | | | HVLSDLSTKLSSNALVFRICHSSVYIWPSSDINTIP | |
| | | | | | GELTDASACKTILRFIQFEPEEDIKRKFMRKKDKK | |
| | | | | | LSDMHQIVNIDLMLEMSTSLAAVTPIIERESGGHH | |
| | | | | | YVNMTLPVDAVISVAPEETWGKVRKLLVDAIHNQ | |
| | | | | | LTDMEKCILKYMKGTSIVVPEPLHFLLPGKKNLVTI | |
| | | | | | SYPSGIPDGQLQAYRKELHDLFNLPHDRPYFKRS | |
| | | | | | NAYHFPDEPYKDGYIRNPHTYLNPPNMETGMIYV | |
| | | | | | VQGIYGYHHYMQDRIDDNGWGCAYRSLQTICSW | |
| | | | | | FKHQGYTERSIPTHREIQQALVDAGDKPATFVGS | |
| | | | | | RQWIGSIEVQLVLNQLIGITSKILFVSQGSEIASQG | |
| | | | | | RELANHFQSEGTPVMIGGGVLAHTILGVAWNEIT | |
| | | | | | GQIKFLILDPHYTGAEDLQVILEKGWCGWKGPDF | |
| | | | | | WNKDAYYNLCLPQRPNMI | |
| 43 | JHUO | IPL | 0.198832435 | CDCA8 | MSPILGYWKIKGLVQPTRLLLEYLEEKYEEHLYER | Full Literal Sequence- |
| | 7018 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAPRKGSSRVANTN | |
| | | | | | SLRRRKLASFLKDFDREVEIRIKQIESDRQNLLKEV | |
| | | | | | DNLYNIEILRLPKALREMNWLDYFALGGNKQALEE | |
| | | | | | AATADLDITEINKLTAEAIQTPLKSAKTRKVIQVDE | |
| | | | | | MIVEEEEEEENERKNLQTARVKRCPPSKKRTQSI | |
| | | | | | QGKGKGKRSSRANTVTPAVGRLEVSMVKPTPGL | |
| | | | | | TPRFDSRVFKTPGLRTPAAGERIYNISGNGSPLAD | |
| | | | | | SKEIFLTVPVGGGESLRLLASDLQRHSIAQLDPEA | |
| | | | | | LGNIKKLSNRLAQICSSIRTHK | |
| 44 | JHUO | IPL | 0.198832435 | CDCA8 | MAPRKGSSRVANTNSLRRRKLASFLKDFDREVEI | Core Human ORF Only |
| | 7018 | RFS | | | RIKQIESDRQNLLKEVDNLYNIEILRLPKALREMN | |
| | | | | | WLDYFALGGNKQALEEAATADLDITEINKLTAEAI | |
| | | | | | QTPLKSAKTRKVIQVDEMIVEEEEEEENERKNLQT | |
| | | | | | ARVKRCPPSKKRTQSIQGKGKGKRSSRANTVTP | |
| | | | | | AVGRLEVSMVKPTPGLTPRFDSRVFKTPGLRTPA | |
| | | | | | AGERIYNISGNGSPLADSKEIFLTVPVGGGESLRL | |
| | | | | | LASDLQRHSIAQLDPEALGNIKKLSNRLAQICSSIR | |
| | | | | | THK | |
| 45 | JHUO | IPL | 0.49987898104 | TMEM2 | MSPILGYWKIKGLVQPTRLLLEYLEEKYEEHLYER | Full Literal Sequence- |
| | 7406 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTVQRLVAAAVLVAL | |
| | | | | | VSLILNNVAAFTSNWVCQTLEDGRRRSVGLWRS | |
| | | | | | CWLVDRTRGGPSPGARAGQVDAHDCEALGWGS | |
| | | | | | EAAGFQESRGTVKLQFDMMRACNLVATAALTAG | |
| | | | | | QLTFLLGLVGLPLLSPDAPCWEEAMAAAFQLASF | |
| | | | | | VLVIGLVTFYRIGPYTNLSWSCYLNIGACLLATLAA | |
| | | | | | AMLIWNILHKREDCMAPRVIVISRSLTARFRRGLD | |
| | | | | | NDYVESPC | |
| 46 | JHUO | IPL | 0.49987890481 | TMEM2 | MTVQRLVAAAVLVALVSLILNNVAAFTSNVWCQT | Core Human ORF Only |
| | 7406 | RFS | | | LEDGRRRSVGLWRSCWLVDRTRGGPSPGARAG | |
| | | | | | QVDAHDCEALGWGSEAAGFQESRGTVKLQFDM | |
| | | | | | MRACNLVATAALTAGQLTFLLGLVGLPLLSPDAP | |
| | | | | | CWEEAMAAAFQLASFVLVIGLVTFYRIGPYTNLS | |
| | | | | | WSCYLNIGACLLATLAAAMLIWNILHKREDCMAPR | |
| | | | | | VIVISRSLTARFRRGLDNDYVESPC | |
| 47 | JHUO | IPL | 0.128505174 | DIABL | MSPILGYWKIKGLVQPTRLLLEYLEEKYEEHLYER | Full Literal Sequence- |
| | 7799 | RFS | | O | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAALKSWLSRSVTS | |
| | | | | | FFRYRQCLCVPVVANFKKRCFSELIRPWHKTVTI | |
| | | | | | GFGVTLCAVPIAQKSEPHSLSSEALMRRAVSLVT | |
| | | | | | DSTSTFLSQTTYALIEAITEYTKAVYTLTSLYRQYT | |
| | | | | | SLLGKMNSEEEDEVWQVIIGARAEMTSKHQEYLK | |
| | | | | | LETTWMTAVGLSEMAAEAAYQTGADQASITARN | |
| | | | | | HIQLVKLQVEEVHQLSRKAETKLAEAQIEELRQKT | |
| | | | | | QEEGEERAESEQEAYLRED | |
| 48 | JHUO | IPL | 0.128505174 | DIABL | MAALKSWLSRSVTSFFRYRQCLCVPVVANFKKR | Core Human ORF Only |
| | 7799 | RFS | | o | CFSELIRPWHKTVTIGFGVTLCAVPIAQKSEPHSL | |
| | | | | | SSEALMRRAVSLVTDSTSTFLSQTTYALIEAITEYT | |
| | | | | | KAVYTLTSLYRQYTSLLGKMNSEEEDEVWQVIIG | |
| | | | | | ARAEMTSKHQEYLKLETTWMTAVGLSEMAAEAA | |
| | | | | | YQTGADQASITARNHIQLVKLQVEEVHQLSRKAE | |
| | | | | | TKLAEAQIEELRQKTQEEGEERAESEQEAYLRED | |
| 49 | JHUO | IPL | 0.116329562 | PHTF2 | MSPILGYWKIKGLVQPTRLLLEYLEEKYEEHLYER | Full Literal Sequence- |
| | 8221 | RFS | | _frag | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLLLGTVHCQIVSTR | |
| | | | | | TPKPPLSTGGKRRRKLRKAAHLEVHREGDGSST | |
| | | | | | TDNTQEGAVQNHGTSTSHSVGTVFRDLWHAAFF | |
| | | | | | LSGSKKAKNSIDKSTETDNGYVSLDGKKTVKSGE | |
| | | | | | DGIQNHEPQCETIRPEETAWNTGTLRNGPSKDTQ | |
| | | | | | RTITNVSDEVSSEEGPETGYSLRRHVDRTSEGVL | |
| | | | | | RNRKSHHYKKHYPNEDAPKSGTSCSSRCSSSRQ | |
| | | | | | DSESARPESETEDVLWEDLLHCAECHSSCTSET | |
| | | | | | DVENHQINPCVKKEYRDDPFHQSHLPWLHSSHP | |
| | | | | | GLEKISAIVWEGNDCKKADMSVLEISGMIMNRVSF | |
| 50 | JHUO | IPL | 0.116329562 | PHTF2 | MLLLGTVHCQIVSTRTPKPPLSTGGKRRRKLRKA | Core Human ORF Only |
| | 8221 | RFS | | _frag | AHLEVHREGDGSSTTDNTQEGAVQNHGTSTSHS | |
| | | | | | VGTVFRDLWHAAFFLSGSKKAKNSIDKSTETDNG | |
| | | | | | YVSLDGKKTVKSGEDGIQNHEPQCETIRPEETAW | |
| | | | | | NTGTLRNGPSKDTQRTITNVSDEVSSEEGPETGY | |
| | | | | | SLRRHVDRTSEGVLRNRKSHHYKKHYPNEDAPK | |
| | | | | | SGTSCSSRCSSSRQDSESARPESETEDVLWEDL | |
| | | | | | LHCAECHSSCTSETDVENHQINPCVKKEYRDDPF | |
| | | | | | HQSHLPWLHSSHPGLEKISAIVWEGNDCKKADM | |
| | | | | | SVLEISGMIMNRVSF | |
| 51 | JHUO | IPI_ | 0.396626912 | PSKH1 | MSPILGYWKIKGLVQPTRLLLEYLEEKYEEHLYER | Full Literal Sequence- |
| | 9956 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGCGTSKVLPEPPK | |
| | | | | | DVQLDLVKKVEPFSGTKSDVYKHFITEVDSVGPV | |
| | | | | | KAGFPAASQYAHPCPGPPTAGHTEPPSEPPRRA | |
| | | | | | RVAKYRAKFDPRVTAKYDIKALIGRGSFSRVVRVE | |
| | | | | | HRATRQPYAIKMIETKYREGREVCESELRVLRRV | |
| | | | | | RHANIIQLVEVFETQERVYMVMELATGGELFDRII | |
| | | | | | AKGSFTERDATRVLQMVLDGVRYLHALGITHRDL | |
| | | | | | KPENLLYYHPGTDSKIIITDFGLASARKKGDDCLM | |
| | | | | | KTTCGTPEYIAPEVLVRKPYTNSVDMWALGVIAYI | |
| | | | | | LLSGTMPFEDDNRTRLYRQILRGKYSYSGEPWPS | |
| | | | | | VSNLAKDFIDRLLTVDPGARMTALQALRHPVWVS | |
| | | | | | MAASSSMKNLHRSISQNLLKRASSRCQSTKSAQS | |
| | | | | | TRSSRSTRSNKSRRVRERELRELNLRYQQQYNG | |
| 52 | JHUO | IPI_ | 0.396626912 | PSKH1 | MGCGTSKVLPEPPKDVQLDLVKKVEPFSGTKSD | Core Human ORF Only |
| | 9956 | RFS | | | VYKHFITEVDSVGPVKAGFPAASQYAHPCPGPPT | |
| | | | | | AGHTEPPSEPPRRARVAKYRAKFDPRVTAKYDIK | |
| | | | | | ALIGRGSFSRVVRVEHRATRQPYAIKMIETKYREG | |
| | | | | | REVCESELRVLRRVRHANIIQLVEVFETQERVYM | |
| | | | | | VMELATGGELFDRIIAKGSFTERDATRVLQMVLD | |
| | | | | | GVRYLHALGITHRDLKPENLLYYHPGTDSKIIITDF | |
| | | | | | GLASARKKGDDCLMKTTCGTPEYIAPEVLVRKPY | |
| | | | | | TNSVDMWALGVIAYILLSGTMPFEDDNRTRLYRQI | |
| | | | | | LRGKYSYSGEPWPSVSNLAKDFIDRLLTVDPGAR | |
| | | | | | MTALQALRHPVWVSMAASSSMKNLHRSISQNLL | |
| | | | | | KRASSRCQSTKSAQSTRSSRSTRSNKSRRVRER | |
| | | | | | ELRELNLRYQQQYNG | |
| 53 | JHU1 | IPL | 0.192544726 | ASPH | MSPILGYWKIKGLVQPTRLLLEYLEEKYEEHLYER | Full Literal Sequence- |
| | 0083 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAQRKNAKSSGNSS | |
| | | | | | SSGSGSGSTSAGSSSPGARRETKHGGHKNGRK | |
| | | | | | GGLSGTSFFTWFMVIALLGVWTSVAVVWFDLVD | |
| | | | | | YEEVLAKAKDFRYNLSEVLQGKLGIYDADGDGDF | |
| | | | | | DVDDAKVLLGLTKDGSNENIDSLEEVLNILAEESS | |
| | | | | | DWFYGFLSFLYDIMTPFEMLEEEEEESETADGVD | |
| | | | | | GTSQNEGVQGKTCVILDLHNQ | |
| 54 | JHU1 | IPL | 0.192544726 | ASPH | MAQRKNAKSSGNSSSSGSGSGSTSAGSSSPGA | Core Human ORF Only |
| | 0083 | RFS | | | RRETKHGGHKNGRKGGLSGTSFFTWFMVIALLG | |
| | | | | | VWTSVAVVWFDLVDYEEVLAKAKDFRYNLSEVL | |
| | | | | | QGKLGIYDADGDGDFDVDDAKVLLGLTKDGSNE | |
| | | | | | NIDSLEEVLNILAEESSDWFYGFLSFLYDIMTPFE | |
| | | | | | MLEEEEEESETADGVDGTSQNEGVQGKTCVILDL | |
| | | | | | HNQ | |
| 55 | JHU1 | IPL | 0.593053392 | ZNF71 | MSPILGYWKIKGLVQPTRLLLEYLEEKYEEHLYER | Full Literal Sequence- |
| | 0827 | RFS | | 0 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEGFMDSGTQTDAV | |
| | | | | | VVLSLAQAAVLGLVSENELFGATISAEAFYPDLGP | |
| | | | | | ELSGAAMGEPEPPGPDVYQLACNGRALEEPAEE | |
| | | | | | EVLEVEAACEKHTRRKTRPPVRLVPKVKFEKVEE | |
| | | | | | EEQEVYEVSVPGDDKDAGPAEAPAEAASGGCDA | |
| | | | | | LVQSSAVKMIDLSAFSRKPRTLRHLPRTPRPELNV | |
| | | | | | APYDPHFPAPARDGFPEPSMALPGPEALPTECG | |
| | | | | | FEPPHLAPLSDPEAPSMESPEPVKPEQGFVWQE | |
| | | | | | ASEFEADTAGSTVERHKKAQLDRLDINVQIDDSYL | |
| | | | | | VEAGDRQKRWQCRMCEKSYTSKYNLVTHILGHN | |
| | | | | | GIKPHSCPHCSKLFKQPSHLQTHLLTHQGTRPHK | |
| | | | | | CQVCHKAFTQTSHLKRHMLLHSEVKPYSCHFCG | |
| | | | | | RGFAYPSELKAHEVKHESGRCHVCVECGLDFST | |
| | | | | | LTQLKRHLASHQGPTLYQCLECDKSFHYRSQLQ | |
| | | | | | NHMLKHQNVRPFVCTECGMEFSQIHHLKQHSLT | |
| | | | | | HKGVKEFKCEVCGREFTLQANMKRHMLIHTSVR | |
| | | | | | PYQCHICFKTFVQKQTLKTHMIVHSPVKPFKCKV | |
| | | | | | CGKSFNRMYNLLGHMHLHAGSKPFKCPYCSSKF | |
| | | | | | NLKGNLSRHMKVKHGVMDIGLDSQDPMMELTGT | |
| | | | | | DPSELDGQQEMEDFEENAYSYASVDSSAEASVL | |
| | | | | | TEQAMKEMAYYNVL | |
| 56 | JHU1 | IPL_ | 0.593053392 | ZNF71 | MEGFMDSGTQTDAVVVLSLAQAAVLGLVSENELF | Core Human ORF Only |
| | 0827 | RFS | | 0 | GATISAEAFYPDLGPELSGAAMGEPEPPGPDVYQ | |
| | | | | | LACNGRALEEPAEEEVLEVEAACEKHTRRKTRPP | |
| | | | | | VRLVPKVKFEKVEEEEQEVYEVSVPGDDKDAGP | |
| | | | | | AEAPAEAASGGCDALVQSSAVKMIDLSAFSRKPR | |
| | | | | | TLRHLPRTPRPELNVAPYDPHFPAPARDGFPEPS | |
| | | | | | MALPGPEALPTECGFEPPHLAPLSDPEAPSMESP | |
| | | | | | EPVKPEQGFVWQEASEFEADTAGSTVERHKKAQ | |
| | | | | | LDRLDINVQIDDSYLVEAGDRQKRWQCRMCEKS | |
| | | | | | YTSKYNLVTHILGHNGIKPHSCPHCSKLFKQPSHL | |
| | | | | | QTHLLTHQGTRPHKCQVCHKAFTQTSHLKRHML | |
| | | | | | LHSEVKPYSCHFCGRGFAYPSELKAHEVKHESG | |
| | | | | | RCHVCVECGLDFSTLTQLKRHLASHQGPTLYQCL | |
| | | | | | ECDKSFHYRSQLQNHMLKHQNVRPFVCTECGM | |
| | | | | | EFSQIHHLKQHSLTHKGVKEFKCEVCGREFTLQA | |
| | | | | | NMKRHMLIHTSVRPYQCHICFKTFVQKQTLKTHMI | |
| | | | | | VHSPVKPFKCKVCGKSFNRMYNLLGHMHLHAGS | |
| | | | | | KPFKCPYCSSKFNLKGNLSRHMKVKHGVMDIGL | |
| | | | | | DSQDPMMELTGTDPSELDGQQEMEDFEENAYS | |
| | | | | | YASVDSSAEASVLTEQAMKEMAYYNVL | |
| 57 | JHU1 | IPL | 0.0757397 | RHBDF | MSPILGYWKIKGLVQPTRLLLEYLEEKYEEHLYER | Full Literal Sequence- |
| | 1580 | RFS | 7 | 2 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASADKNGGSVSSV | |
| | | | | | SSSRLQSRKPPNLSITIPPPEKETQAPGEQDSMLP | |
| | | | | | EGFQNRRLKKSQPRTWASHTTACPPSFLPKRKN | |
| | | | | | PAYLKSVSLQEPRSRWQESSEKRPGFRRQASLS | |
| | | | | | QSIRKGAAQWFGVSGDWEGQRQQWQRRSLHH | |
| | | | | | CSMRYGRLKASCQRDLELPSQEAPSFQGTESPK | |
| | | | | | PCKMPKIVDPLARGRAFRHPEEMDRPHALHPPLT | |
| | | | | | PGVLSLTSFTSVRSGYSHLPRRKRMSVAHMSLQ | |
| | | | | | AAAALLKGRSVLDATGQRCRVVKRSFAFPSFLEE | |
| | | | | | DVVDGADTFDSSFFSKEEMSSMPDDVFESPPLS | |
| | | | | | ASYFRGIPHSASPVSPDGVQIPLKEYGRAPVPGP | |
| | | | | | RRGKRIASKVKHFAFDRKKRHYGLGVVGNWLNR | |
| | | | | | SYRRSISSTVQRQLESFDSHRPYFTYWLTFVHVII | |
| | | | | | TLLVICTYGIAPVGFAQHVTTQLVLRNKGVYESVK | |
| | | | | | YIQQENFWVGPSSIDLIHLGAKFSPCIRKDGQIEQ | |
| | | | | | LVLRERDLERDSGCCVQNDHSGCIQTQRKDCSE | |
| | | | | | TLATFVKWQDDTGPPMDKSDLGQKRTSGAVCH | |
| | | | | | QDPRTCEEPASSGAHIWPDDITKWPICTEQARSN | |
| | | | | | HTGFLHMDCEIKGRPCCIGTKGSCEITTREYCEF | |
| | | | | | MHGYFHEEATLCSQVHCLDKVCGLLPFLNPEVPD | |
| | | | | | QFYRLWLSLFLHAGVVHCLVSVVFQMTILRDLEKL | |
| | | | | | AGWHRIAIIFILSGITGNLASAIFLPYRAEVGPAGS | |
| | | | | | QFGLLACLFVELFQSWPLLERPWKAFLNLSAIVLF | |
| | | | | | LFICGLLPWIDNIAHIFGFLSGLLLAFAFLPYITFGT | |
| | | | | | SDKYRKRALILVSLLAFAGLFAALVLWLYIYPINWP | |
| | | | | | WIEHLTCFPFTSRFCEKYELDQVLH | |
| 58 | JHU1 | IPL | 0.0757397 | RHBDF | MASADKNGGSVSSVSSSRLQSRKPPNLSITIPPP | Core Human ORF Only |
| | 1580 | RFS | 7 | 2 | EKETQAPGEQDSMLPEGFQNRRLKKSQPRTWA | |
| | | | | | SHTTACPPSFLPKRKNPAYLKSVSLQEPRSRWQ | |
| | | | | | ESSEKRPGFRRQASLSQSIRKGAAQWFGVSGD | |
| | | | | | WEGQRQQWQRRSLHHCSMRYGRLKASCQRDL | |
| | | | | | ELPSQEAPSFQGTESPKPCKMPKIVDPLARGRAF | |
| | | | | | RHPEEMDRPHALHPPLTPGVLSLTSFTSVRSGYS | |
| | | | | | HLPRRKRMSVAHMSLQAAAALLKGRSVLDATGQ | |
| | | | | | RCRVVKRSFAFPSFLEEDVVDGADTFDSSFFSKE | |
| | | | | | EMSSMPDDVFESPPLSASYFRGIPHSASPVSPDG | |
| | | | | | VQIPLKEYGRAPVPGPRRGKRIASKVKHFAFDRK | |
| | | | | | KRHYGLGVVGNWLNRSYRRSISSTVQRQLESFD | |
| | | | | | SHRPYFTYWLTFVHVIITLLVICTYGIAPVGFAQHV | |
| | | | | | TTQLVLRNKGVYESVKYIQQENFVWGPSSIDLIHL | |
| | | | | | GAKFSPCIRKDGQIEQLVLRERDLERDSGCCVQN | |
| | | | | | DHSGCIQTQRKDCSETLATFVKWQDDTGPPMDK | |
| | | | | | SDLGQKRTSGAVCHQDPRTCEEPASSGAHIWPD | |
| | | | | | DITKWPICTEQARSNHTGFLHMDCEIKGRPCCIGT | |
| | | | | | KGSCEITTREYCEFMHGYFHEEATLCSQVHCLDK | |
| | | | | | VCGLLPFLNPEVPDQFYRLWLSLFLHAGVVHCLV | |
| | | | | | SVVFQMTILRDLEKLAGWHRIAIIFILSGITGNLASA | |
| | | | | | IFLPYRAEVGPAGSQFGLLACLFVELFQSWPLLER | |
| | | | | | PWKAFLNLSAIVLFLFICGLLPWIDNIAHIFGFLSGL | |
| | | | | | LLAFAFLPYITFGTSDKYRKRALILVSLLAFAGLFA | |
| | | | | | ALVLWLYIYPINWPWIEHLTCFPFTSRFCEKYELD | |
| | | | | | QVLH | |
| 59 | JHU1 | IPL_ | 0.142119811 | SLC6A | MSPILGYWKIKGLVQPTRLLLEYLEEKYEEHLYER | Full Literal Sequence- |
| | 2427 | RFS | 03 | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTAEKALPLGNGKA | |
| | | | | | AEEARESEAPGGGCSSGGAAPARHPRVKRDKAV | |
| | | | | | HERGHWNNKVEFVLSVAGEIIGLGNVWRFPYLCY | |
| | | | | | KNGGGAFLIPYVVFFICCGIPVFFLETALGQFTSE | |
| | | | | | GGITCWRKVCPLFEGIGYATQVIEAHLNVYYIIILA | |
| | | | | | WAIFYLSNCFTTELPWATCGHEWNTENCVEFQK | |
| | | | | | LNVSNYSHVSLQNATSPVMEFWE | |
| 60 | JHU1 | IPL | 0.142119803 | SLC6A | MTAEKALPLGNGKAAEEARESEAPGGGCSSGGA | Core Human ORF Only |
| | 2427 | RFS | | 11 | APARHPRVKRDKAVHERGHWNNKVEFVLSVAGE | |
| | | | | | HGLGNVWRFPYLCYKNGGGAFLIPYVVFFICCGIP | |
| | | | | | VFFLETALGQFTSEGGITCWRKVCPLFEGIGYAT | |
| | | | | | QVIEAHLNVYYIIILAWAIFYLSNCFTTELPWATCG | |
| | | | | | HEWNTENCVEFQKLNVSNYSHVSLQNATSPVME | |
| | | | | | FWE | |
| 61 | JHU1 | IPL | 0.273227227 | ASCL1 | MSPILGYWKIKGLVQPTRLLLEYLEEKYEEHLYER | Full Literal Sequence- |
| | 2735 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMESSAKMESGGAGQ | |
| | | | | | QPQPQPQQPFLPPAACFFATAAAAAAAAAAAAA | |
| | | | | | QSAQQQQQQQQQQQQAPQLRPAADGQPSGGG | |
| | | | | | HKSAPKQVKRQRSSSPELMRCKRRLNFSGFGYS | |
| | | | | | LPQQQPAAVARRNERERNRVKLVNLGFATLREH | |
| | | | | | VPNGAANKKMSKVETLRSAVEYIRALQQLLDEHD | |
| | | | | | AVSAAFQAGVLSPTISPNYSNDLNSMAGSPVSSY | |
| | | | | | SSDEGSYDPLSPEEQELLDFTNWF | |
| 62 | JHU1 | IPL | 0.273227227 | ASCL1 | MESSAKMESGGAGQQPQPQPQQPFLPPAACFF | Core Human ORF Only |
| | 2735 | RFS | | | ATAAAAAAAAAAAAAQSAQQQQQQQQQQQQAP | |
| | | | | | QLRPAADGQPSGGGHKSAPKQVKRQRSSSPEL | |
| | | | | | MRCKRRLNFSGFGYSLPQQQPAAVARRNERER | |
| | | | | | NRVKLVNLGFATLREHVPNGAANKKMSKVETLRS | |
| | | | | | AVEYIRALQQLLDEHDAVSAAFQAGVLSPTISPNY | |
| | | | | | SNDLNSMAGSPVSSYSSDEGSYDPLSPEEQELL | |
| | | | | | DFTNWF | |
| 63 | JHU1 | IPL | 0.419791316 | CNP | MSPILGYWKIKGLVQPTRLLLEYLEEKYEEHLYER | Full Literal Sequence- |
| | 2847 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSSSGAKDKPELQF | |
| | | | | | PFLQDEDTVATLLECKTLFILRGLPGSGKSTLARVI | |
| | | | | | VDKYRDGTKMVSADAYKITPGARGAFSEEYKRLD | |
| | | | | | EDLAAYCRRRDIRILVLDDTNHERERLEQLFEMAD | |
| | | | | | QYQYQVVLVEPKTAWRLDCAQLKEKNQWQLSA | |
| | | | | | DDLKKLKPGLEKDFLPLYFGWFLTKKSSETLRKA | |
| | | | | | GQVFLEELGNHKAFKKELRQFVPGDEPREKMDL | |
| | | | | | VTYFGKRPPGVLHCTTKFCDYGKAPGAEEYAQQ | |
| | | | | | DVLKKSYSKAFTLTISALFVTPKTTGARVELSEQQ | |
| | | | | | LQLWPSDVDKLSPTDNLPRGSRAHITLGCAADVE | |
| | | | | | AVQTGLDLLEILRQEKGGSRGEEVGELSRGKLYS | |
| | | | | | LGNGRWMLTLAKNMEVRAIFTGYYGKGKPVPTQ | |
| | | | | | GSRKGGALQSCTII | |
| 64 | JHU1 | IPL_ | 0.419791316 | CNP | MSSSGAKDKPELQFPFLQDEDTVATLLECKTLFIL | Core Human ORF Only |
| | 2847 | RFS | | | RGLPGSGKSTLARVIVDKYRDGTKMVSADAYKIT | |
| | | | | | PGARGAFSEEYKRLDEDLAAYCRRRDIRILVLDDT | |
| | | | | | NHERERLEQLFEMADQYQYQVVLVEPKTAWRLD | |
| | | | | | CAQLKEKNQWQLSADDLKKLKPGLEKDFLPLYFG | |
| | | | | | WFLTKKSSETLRKAGQVFLEELGNHKAFKKELRQ | |
| | | | | | FVPGDEPREKMDLVTYFGKRPPGVLHCTTKFCD | |
| | | | | | YGKAPGAEEYAQQDVLKKSYSKAFTLTISALFVTP | |
| | | | | | KTTGARVELSEQQLQLWPSDVDKLSPTDNLPRG | |
| | | | | | SRAHITLGCAADVEAVQTGLDLLEILRQEKGGSR | |
| | | | | | GEEVGELSRGKLYSLGNGRWMLTLAKNMEVRAI | |
| | | | | | FTGYYGKGKPVPTQGSRKGGALQSCTII | |
| 65 | JHU1 | IPL | 0.289636 | PPP1R | MSPILGYWKIKGLVQPTRLLLEYLEEKYEEHLYER | Full Literal Sequence- |
| | 3275 | RFS | | 12B | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAELEHLGGKRAES | |
| | | | | | ARMRRAEQLRRWRGSLTEQEPAERRGAGRQPL | |
| | | | | | TRRGSPRVRFEDGAVFLAACSSGDTDEVRKLLA | |
| | | | | | RGADINTVNVDGLTALHQACIDENLDMVKFLVEN | |
| | | | | | RANVNQQDNEGWTPLHAAASCGYLNIAEYFINHG | |
| | | | | | ASVGIVNSEGEVPSDLAEEPAMKDLLLEQVKKQG | |
| | | | | | VDLEQSRKEEEQQMLQDARQWLNSGKIEDVRQA | |
| | | | | | RSGATALHVAAAKGYSEVLRLLIQAGYELNVQDY | |
| | | | | | DGWTPLHAAAHWGVKEACSILAEALCDMDIRNKL | |
| | | | | | GQTPFDVADEGLVEHLELLQKKQNVLRSEKETRN | |
| | | | | | KLIESDLNSKIQSGFFKNKEKMLYEEETPKSQEME | |
| | | | | | EENKESSSSSSEEEEGEDEASESETEKEAVLFWP | |
| | | | | | F | |
| 66 | JHU1 | IPL | 0.289636 | PPP1R | MAELEHLGGKRAESARMRRAEQLRRWRGSLTE | Core Human ORF Only |
| | 3275 | RFS | | 12B | QEPAERRGAGRQPLTRRGSPRVRFEDGAVFLAA | |
| | | | | | CSSGDTDEVRKLLARGADINTVNVDGLTALHQAC | |
| | | | | | IDENLDMVKFLVENRANVNQQDNEGWTPLHAAA | |
| | | | | | SCGYLNIAEYFINHGASVGIVNSEGEVPSDLAEEP | |
| | | | | | AMKDLLLEQVKKQGVDLEQSRKEEEQQMLQDAR | |
| | | | | | QWLNSGKIEDVRQARSGATALHVAAAKGYSEVL | |
| | | | | | RLLIQAGYELNVQDYDGWTPLHAAAHWGVKEAC | |
| | | | | | SILAEALCDMDIRNKLGQTPFDVADEGLVEHLELL | |
| | | | | | QKKQNVLRSEKETRNKLIESDLNSKIQSGFFKNKE | |
| | | | | | KMLYEEETPKSQEMEEENKESSSSSSEEEEGED | |
| | | | | | EASESETEKEAVLFWPF | |
| 67 | JHU1 | IPL | 0.42408019 | NEDD4 | MSPILGYWKIKGLVQPTRLLLEYLEEKYEEHLYER | Full Literal Sequence- |
| | 3360 | RFS | 9 | L | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMATGLGEPVYGLSE | |
| | | | | | DEGESRILRVKVVSGIDLAKKDIFGASDPYVKLSL | |
| | | | | | YVADENRELALVQTKTIKKTLNPKWNEEFYFRVN | |
| | | | | | PSNHRLLFEVFDENRLTRDDFLGQVDVPLSHLPT | |
| | | | | | EDPTMERPYTFKDFLLRPRSHKSRVKGFLRLKMA | |
| | | | | | YMPKNGGQDEENSDQRDDMEHGWEVVDSNDS | |
| | | | | | ASQHQEELPPPPLPPGWEEKVDNLGRTYYVNHN | |
| | | | | | NRTTQWHRPSLMDVSSESDNNIRQINQEAAHRR | |
| | | | | | FRSRRHISEDLEPEPSEGGDVPEPWETISEEVNIA | |
| | | | | | GDSLGLALPPPPASPGSRTSPQELSEELSRRLQIT | |
| | | | | | PDSNGEQFSSLIQREPSSRLRSCSVTDAVAEQGH | |
| | | | | | LPPLAEDGASGSATNSNNHLIEPQIRRPRSLSSPT | |
| | | | | | VTLSAPLEGAKDSPVRRAVKDTLSNPQSPQPSPY | |
| | | | | | NSPKPQHKVTQSFLPPGWEMRIAPNGRPFFIDHN | |
| | | | | | TKTTTWEDPRLKFPVHMRSKTSLNPNDLGPLPPG | |
| | | | | | WEERIHLDGRTFYIDHNSKITQWEDPRLQNPAITG | |
| | | | | | PAVPYSREFKQKYDYFRKKLKKPADIPNRFEMKL | |
| | | | | | HRNNIFEESYRRIMSVKRPDVLKARLWIEFESEKG | |
| | | | | | LDYGGVAREWFFLLSKEMFNPYYGLFEYSATDN | |
| | | | | | YTLQINPNSGLCNEDHLSYFTFIGRVAGLAVFHGK | |
| | | | | | LLDGFFIRPFYKMMLGKQITLNDMESVDSEYYNSL | |
| | | | | | KWILENDPTELDLMFCIDEENFGQTYQVDLKPNG | |
| | | | | | SEIMVTNENKREYIDLVIQWRFVNRVQKQMNAFL | |
| | | | | | EGFTELLPIDLIKIFDENELELLMCGLGDVDVNDW | |
| | | | | | RQHSIYKNGYCPNHPVIQWFWKAVLLMDAEKRIR | |
| | | | | | LLQFVTGTSRVPMNGFAELYGSNGPQLFTIEQW | |
| | | | | | GSPEKLPRAHTCFNRLDLPPYETFEDLREKLLMA | |
| | | | | | VENAQGFEGVD | |
| 68 | JHU1 | IPL_ | 0.424080199 | NEDD4 | MATGLGEPVYGLSEDEGESRILRVKVVSGIDLAK | Core Human ORF Only |
| | 3360 | RFS | | L | KDIFGASDPYVKLSLYVADENRELALVQTKTIKKTL | |
| | | | | | NPKWNEEFYFRVNPSNHRLLFEVFDENRLTRDD | |
| | | | | | FLGQVDVPLSHLPTEDPTMERPYTFKDFLLRPRS | |
| | | | | | HKSRVKGFLRLKMAYMPKNGGQDEENSDQRDD | |
| | | | | | MEHGWEVVDSNDSASQHQEELPPPPLPPGWEE | |
| | | | | | KVDNLGRTYYVNHNNRTTQWHRPSLMDVSSESD | |
| | | | | | NNIRQINQEAAHRRFRSRRHISEDLEPEPSEGGD | |
| | | | | | VPEPWETISEEVNIAGDSLGLALPPPPASPGSRTS | |
| | | | | | PQELSEELSRRLQITPDSNGEQFSSLIQREPSSRL | |
| | | | | | RSCSVTDAVAEQGHLPPLAEDGASGSATNSNNH | |
| | | | | | LIEPQIRRPRSLSSPTVTLSAPLEGAKDSPVRRAV | |
| | | | | | KDTLSNPQSPQPSPYNSPKPQHKVTQSFLPPGW | |
| | | | | | EMRIAPNGRPFFIDHNTKTTTWEDPRLKFPVHMR | |
| | | | | | SKTSLNPNDLGPLPPGWEERIHLDGRTFYIDHNS | |
| | | | | | KITQWEDPRLQNPAITGPAVPYSREFKQKYDYFR | |
| | | | | | KKLKKPADIPNRFEMKLHRNNIFEESYRRIMSVKR | |
| | | | | | PDVLKARLWIEFESEKGLDYGGVAREWFFLLSKE | |
| | | | | | MFNPYYGLFEYSATDNYTLQINPNSGLCNEDHLS | |
| | | | | | YFTFIGRVAGLAVFHGKLLDGFFIRPFYKMMLGKQ | |
| | | | | | ITLNDMESVDSEYYNSLKWILENDPTELDLMFCID | |
| | | | | | EENFGQTYQVDLKPNGSEIMVTNENKREYIDLVIQ | |
| | | | | | WRFVNRVQKQMNAFLEGFTELLPIDLIKIFDENEL | |
| | | | | | ELLMCGLGDVDVNDWRQHSIYKNGYCPNHPVIQ | |
| | | | | | WFWKAVLLMDAEKRIRLLQFVTGTSRVPMNGFA | |
| | | | | | ELYGSNGPQLFTIEQWGSPEKLPRAHTCFNRLDL | |
| | | | | | PPYETFEDLREKLLMAVENAQGFEGVD | |
| 69 | JHU1 | IPI_ | 0.229880158 | NLK | MSPILGYWKIKGLVQPTRLLLEYLEEKYEEHLYER | Full Literal Sequence- |
| | 3473 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAYNGGTSAAATG | |
| | | | | | HHHHHHHHLPHLPPPHLHHHHHPQHHLHPGSAA | |
| | | | | | AVHPVQQHTSSAAAAAAAAAAAAAMLNPGQQQP | |
| | | | | | YFPSPAPGQAPGPAAAAPAQVQAAAAATVKAHH | |
| | | | | | HQHSHHPQQQLDIEPDRPIGYGAFGVVWSVTDP | |
| | | | | | RDGKRVALKKMPNVFQNLVSCKRVFRELKMLCF | |
| | | | | | FKHDNVLSALDILQPPHIDYFEEIYVVTELMQSDLH | |
| | | | | | KIIVSPQPLSSDHVKVFLYQILRGLKYLHSAGILHR | |
| | | | | | DIKPGNLLVNSNCVLKICDFGLARVEELDESRHMT | |
| | | | | | QEVVTQYYRAPEILMGSRHYSNAIDIWSVGCIFAE | |
| | | | | | LLGRRILFQAQSPIQQLDLITDLLGTPSLEAMRTAC | |
| | | | | | EGAKAHILRGPHKQPSLPVLYTLSSQATHEAVHLL | |
| | | | | | CRMLVFDPSKRISAKDALAHPYLDEGRLRYHTCM | |
| | | | | | CKCCFSTSTGRVYTSDFEPVTNPKFDDTFEKNLS | |
| | | | | | SVRQVKEIIHQFILEQQKGNRVPLCINPQSAAFKS | |
| | | | | | FISSTVAQPSEMPPSPLVWE | |
| 70 | JHU1 | IPI_ | 0.229880158 | NLK | MAAYNGGTSAAATGHHHHHHHHLPHLPPPHLHH | Core Human ORF Only |
| | 3473 | RFS | | | HHHPQHHLHPGSAAAVHPVQQHTSSAAAAAAAA | |
| | | | | | AAAAAMLNPGQQQPYFPSPAPGQAPGPAAAAPA | |
| | | | | | QVQAAAAATVKAHHHQHSHHPQQQLDIEPDRPI | |
| | | | | | GYGAFGVVWSVTDPRDGKRVALKKMPNVFQNLV | |
| | | | | | SCKRVFRELKMLCFFKHDNVLSALDILQPPHIDYF | |
| | | | | | EEIYVVTELMQSDLHKIIVSPQPLSSDHVKVFLYQI | |
| | | | | | LRGLKYLHSAGILHRDIKPGNLLVNSNCVLKICDF | |
| | | | | | GLARVEELDESRHMTQEVVTQYYRAPEILMGSR | |
| | | | | | HYSNAIDIWSVGCIFAELLGRRILFQAQSPIQQLDL | |
| | | | | | ITDLLGTPSLEAMRTACEGAKAHILRGPHKQPSLP | |
| | | | | | VLYTLSSQATHEAVHLLCRMLVFDPSKRISAKDAL | |
| | | | | | AHPYLDEGRLRYHTCMCKCCFSTSTGRVYTSDF | |
| | | | | | EPVTNPKFDDTFEKNLSSVRQVKEIIHQFILEQQK | |
| | | | | | GNRVPLCINPQSAAFKSFISSTVAQPSEMPPSPLV | |
| | | | | | WE | |
| 71 | JHU1 | IPI_ | 0.638957503 | ARHGE | MSPILGYWKIKGLVQPTRLLLEYLEEKYEEHLYER | Full Literal Sequence- |
| | 3694 | RFS | | F3 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | |
| | | | | | PKLVCFKKRIEAIPQIDKYLKSSKYIAWPLQGWQA | ORF, occasional ‘post |
| | | | | | TFGGGDHPPKSDLVPRGSPGISGGGGGILGRGS | stop’ end sequence) |
| | | | | | HHHHHHGGGITSLYKKAGTMVAKDYPFYLTVKRA | |
| | | | | | NCSLELPPASGPAKDAEEPSNKRVKPLSRVTSLA | |
| | | | | | NLIPPVKATPLKRFSQTLQRSISFRSESRPDILAPR | |
| | | | | | PWSRNAAPSSTKRRDSKLWSETFDVCVNQMLTS | |
| | | | | | KEIKRQEAIFELSQGEEDLIEDLKLAKKAYHDPML | |
| | | | | | KLSIMTEQELNQIFGTLDSLIPLHEELLSQLRDVRK | |
| | | | | | PDGSTEHVGPILVGWLPCLSSYDSYCSNQVAAKA | |
| | | | | | LLDHKKQDHRVQDFLQRCLESPFSRKLDLWNFL | |
| | | | | | DIPRSRLVKYPLLLREILRHTPNDNPDQQHLEEAI | |
| | | | | | NIIQGIVAEINTKTGESECRYYKERLLYLEEGQKDS | |
| | | | | | LIDSSRVLCCHGELKNNRGVKLHVFLFQEVLVITR | |
| | | | | | AVTHNEQLCYQLYRQPIPVKDLLLEDLQDGEVRL | |
| | | | | | GGSLRGAFSNNERIKNFFRVSFKNGSQSQTHSL | |
| | | | | | QANDTFNKQQWLNCIRQAKETVLCAAGQAGVLD | |
| | | | | | SEGSFLNPTTGSRELQGETKLEQMDQSDSESDC | |
| | | | | | SMDTSEVSLDCERMEQTDSSCGNSRHGESNV | |
| 72 | JHU1 | IPL_ | 0.638957503 | ARHGE | MVAKDYPFYLTVKRANCSLELPPASGPAKDAEEP | Core Human ORF Only |
| | 3694 | RFS | | F3 | SNKRVKPLSRVTSLANLIPPVKATPLKRFSQTLQR | |
| | | | | | SISFRSESRPDILAPRPWSRNAAPSSTKRRDSKL | |
| | | | | | WSETFDVCVNQMLTSKEIKRQEAIFELSQGEEDLI | |
| | | | | | EDLKLAKKAYHDPMLKLSIMTEQELNQIFGTLDSLI | |
| | | | | | PLHEELLSQLRDVRKPDGSTEHVGPILVGWLPCL | |
| | | | | | SSYDSYCSNQVAAKALLDHKKQDHRVQDFLQRC | |
| | | | | | LESPFSRKLDLWNFLDIPRSRLVKYPLLLREILRHT | |
| | | | | | PNDNPDQQHLEEAINIIQGIVAEINTKTGESECRYY | |
| | | | | | KERLLYLEEGQKDSLIDSSRVLCCHGELKNNRGV | |
| | | | | | KLHVFLFQEVLVITRAVTHNEQLCYQLYRQPIPVK | |
| | | | | | DLLLEDLQDGEVRLGGSLRGAFSNNERIKNFFRV | |
| | | | | | SFKNGSQSQTHSLQANDTFNKQQWLNCIRQAKE | |
| | | | | | TVLCAAGQAGVLDSEGSFLNPTTGSRELQGETKL | |
| | | | | | EQMDQSDSESDCSMDTSEVSLDCERMEQTDSS | |
| | | | | | CGNSRHGESNV | |
| 73 | JHU1 | IPL | 0.222277988 | SKAP1 | MSPILGYWKIKGLVQPTRLLLEYLEEKYEEHLYER | Full Literal Sequence- |
| | 3865 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMQAAALPEEIRWLLE | |
| | | | | | DAEEFLAEGLRNENLSAVARDHRDHILRGFQQIK | |
| | | | | | ARYYWDFQPQGGDIGQDSSDDNHSGTLGLSLTS | |
| | | | | | DAPFLSDYQDEGMEDIVKGAQELDNVIKQGYLEK | |
| | | | | | KSKDHSFFGSEWQKRWCVVSRGLFYYYANEKSK | |
| | | | | | QPKGTFLIKGYSVRMAPHLRRDSKKESCFELTSQ | |
| | | | | | DRRSYEFTATSPAEARDVWDQISFLLKDLSSLTIP | |
| | | | | | YEEDEEEEEKEETYDDIDGFDSPSCGSQCRPTIL | |
| | | | | | PGSVGIKEPTEEKEEEDIYEVLPDEEHDLEEDESG | |
| | | | | | TRRKGVDYASYYQGLWDCHGDQPDELSFQRGD | |
| | | | | | LIRILSKEYNMYGVWWGELNSLVGIVPKEYLTTAF | |
| | | | | | EVEER | |
| 74 | JHU1 | IPL | 0.222277988 | SKAP1 | MQAAALPEEIRWLLEDAEEFLAEGLRNENLSAVA | Core Human ORF Only |
| | 3865 | RFS | | | RDHRDHILRGFQQIKARYYWDFQPQGGDIGQDS | |
| | | | | | SDDNHSGTLGLSLTSDAPFLSDYQDEGMEDIVKG | |
| | | | | | AQELDNVIKQGYLEKKSKDHSFFGSEWQKRWCV | |
| | | | | | VSRGLFYYYANEKSKQPKGTFLIKGYSVRMAPHL | |
| | | | | | RRDSKKESCFELTSQDRRSYEFTATSPAEARDWV | |
| | | | | | DQISFLLKDLSSLTIPYEEDEEEEEKEETYDDIDG | |
| | | | | | FDSPSCGSQCRPTILPGSVGIKEPTEEKEEEDIYE | |
| | | | | | VLPDEEHDLEEDESGTRRKGVDYASYYQGLWDC | |
| | | | | | HGDQPDELSFQRGDLIRILSKEYNMYGVWWGEL | |
| | | | | | NSLVGIVPKEYLTTAFEVEER | |
| 75 | JHU1 | IPL | 0.140211961 | RNF8 | MSPILGYWKIKGLVQPTRLLLEYLEEKYEEHLYER | Full Literal Sequence- |
| | 4147 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAFP | ORF, occasional ‘post |
| | | | | | KLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGEPGFFVTGDRAG | |
| | | | | | GRSWCLRRVGMSAGWLLLEDGCEVTVGRGFGV | |
| | | | | | TYQLVSKICPLMISRNHCVLKQNPEGQWTIMDNK | |
| | | | | | SLNGVWLNRARLEPLRVYSIHQGDYIQLGVPLEN | |
| | | | | | KENAEYEYEVTEEDWETIYPCLSPKNDQMIEKNK | |
| | | | | | ELRTKRKFSLDELAGPGAEGPSNLKSKINKVSCE | |
| | | | | | SGQPVKSQGKGEVASTPSDNLDPKLTALEPSKTT | |
| | | | | | GAPIYPGFPKVTEVHHEQKASNSSASQRSLQMFK | |
| | | | | | VTMSRILRLKIQMQEKHEAVMNVKKQTQKGNSKK | |
| | | | | | VVQMEQELQDLQSQLCAEQAQQQARVEQLEKTF | |
| | | | | | QEEEQHLQGLEIAQGEKDLKQQLAQALQEHWAL | |
| | | | | | MEELNRSKKDFEAIIQAKNKELEQTKEEKEKMQA | |
| | | | | | QKEEVLSHMNDVLENELQCIICSEYFIEAVTLNCA | |
| | | | | | HSFCSYCINEWMKRKIECPICRKDIKSKTYSLVLD | |
| | | | | | NCINKMVNNLSSEVKERRIVLIRERKAKRLF | |
| 76 | JHU1 | IPL_ | 0.140211961 | RNF8 | MGEPGFFVTGDRAGGRSWCLRRVGMSAGWLLL | Core Human ORF Only |
| | 4147 | RFS | | | EDGCEVTVGRGFGVTYQLVSKICPLMISRNHCVL | |
| | | | | | KQNPEGQWTIMDNKSLNGVWLNRARLEPLRVYS | |
| | | | | | IHQGDYIQLGVPLENKENAEYEYEVTEEDWETIYP | |
| | | | | | CLSPKNDQMIEKNKELRTKRKFSLDELAGPGAEG | |
| | | | | | PSNLKSKINKVSCESGQPVKSQGKGEVASTPSDN | |
| | | | | | LDPKLTALEPSKTTGAPIYPGFPKVTEVHHEQKAS | |
| | | | | | NSSASQRSLQMFKVTMSRILRLKIQMQEKHEAVM | |
| | | | | | NVKKQTQKGNSKKVVQMEQELQDLQSQLCAEQ | |
| | | | | | AQQQARVEQLEKTFQEEEQHLQGLEIAQGEKDL | |
| | | | | | KQQLAQALQEHWALMEELNRSKKDFEAIIQAKNK | |
| | | | | | ELEQTKEEKEKMQAQKEEVLSHMNDVLENELQCI | |
| | | | | | ICSEYFIEAVTLNCAHSFCSYCINEWMKRKIECPIC | |
| | | | | | RKDIKSKTYSLVLDNCINKMVNNLSSEVKERRIVLI | |
| | | | | | RERKAKRLF | |
| 77 | JHU1 | IPL | 0.381088655 | ZBTB1 | MSPILGYWKIKGLVQPTRLLLEYLEEKYEEHLYER | Full Literal Sequence- |
| | 4260 | RFS | | 2 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASGVEVLRFQLPG | |
| | | | | | HEAATLRNMNQLRAEERFCDVTIVADSLKFRGHK | |
| | | | | | VILAACSPFLRDQFLLNPSSELQVSLMHSARIVAD | |
| | | | | | LLLSCYTGALEFAVRDIVNYLTAASYLQMEHVVEK | |
| | | | | | CRNALSQFIEPKIGLKEDGVSEASLVSSISATKSLL | |
| | | | | | PPARTPKPAPKPPPPPPLPPPLLRPVKLEFPLDED | |
| | | | | | LELKAEEEDEDEDEDVSDICIVKVESALEVAHRLK | |
| | | | | | PPGGLGGGLGIGGSVGGHLGELAQSSVPPSTVA | |
| | | | | | PPQGVVKACYSLSEDAEGEGLLLIPGGRASVGAT | |
| | | | | | SGLVEAAAVAMAARGAGGSLGAGGSRGPLPGG | |
| | | | | | FSGGNPLKNIKCTKCPEVFQGVEKLVFHMRAQHF | |
| | | | | | IFMCPRCGKQFNHSSNLNRHMNVHRGVKSHSCG | |
| | | | | | ICGKCFTQKSTLHDHLNLHSGARPYRCSYCDVRF | |
| | | | | | AHKPAIRRHLKEQHGKTTAENVLEASVAEINVLIR | |
| 78 | JHU1 | IPL | 0.381088655 | ZBTB1 | MASGVEVLRFQLPGHEAATLRNMNQLRAEERFC | Core Human ORF Only |
| | 4260 | RFS | | 2 | DVTIVADSLKFRGHKVILAACSPFLRDQFLLNPSS | |
| | | | | | ELQVSLMHSARIVADLLLSCYTGALEFAVRDIVNY | |
| | | | | | LTAASYLQMEHVVEKCRNALSQFIEPKIGLKEDGV | |
| | | | | | SEASLVSSISATKSLLPPARTPKPAPKPPPPPPLP | |
| | | | | | PPLLRPVKLEFPLDEDLELKAEEEDEDEDEDVSDI | |
| | | | | | CIVKVESALEVAHRLKPPGGLGGGLGIGGSVGGH | |
| | | | | | LGELAQSSVPPSTVAPPQGVVKACYSLSEDAEGE | |
| | | | | | GLLLIPGGRASVGATSGLVEAAAVAMAARGAGGS | |
| | | | | | LGAGGSRGPLPGGFSGGNPLKNIKCTKCPEVFQ | |
| | | | | | GVEKLVFHMRAQHFIFMCPRCGKQFNHSSNLNR | |
| | | | | | HMNVHRGVKSHSCGICGKCFTQKSTLHDHLNLH | |
| | | | | | SGARPYRCSYCDVRFAHKPAIRRHLKEQHGKTTA | |
| | | | | | ENVLEASVAEINVLIR | |
| 79 | JHU1 | IPL | 0.132214362 | ATP6V | MSPILGYWKIKGLVQPTRLLLEYLEEKYEEHLYER | Full Literal Sequence- |
| | 4366 | RFS | | 1G3 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTSQSQGIHQLLQAE | |
| | | | | | KRAKDKLEEAKKRKGKRLKQAKEEAMVEIDQYR | |
| | | | | | MQRDKEFRLKQSKIMGSQNNLSDEIEEQTLGKIQ | |
| | | | | | ELNGHYNKYMESVMNQLLSMVCDMKPEIHVNYR | |
| | | | | | ATN | |
| 80 | JHU1 | IPL | 0.132214362 | ATP6V | MTSQSQGIHQLLQAEKRAKDKLEEAKKRKGKRLK | Core Human ORF Only |
| | 4366 | RFS | | 1G3 | QAKEEAMVEIDQYRMQRDKEFRLKQSKIMGSQN | |
| | | | | | NLSDEIEEQTLGKIQELNGHYNKYMESVMNQLLS | |
| | | | | | MVCDMKPEIHVNYRATN | |
| 81 | JHU1 | IPL | 0.275263482 | C2orf66 | MSPILGYWKIKGLVQPTRLLLEYLEEKYEEHLYER | Full Literal Sequence- |
| | 4419 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMIIDSSRIPSFTQLHS | |
| | | | | | TMTRAPLLLLCVALVLLGHVNGATVRNEDKWKPL | |
| | | | | | NNPRNRDLFFRRLQAYFKGRGLDLGTFPNPFPTN | |
| | | | | | ENPRPLSFQSELTASASADYEEQKNSFHNYLKG | |
| 82 | JHU1 | IPL | 0.275263482 | C2orf66 | MIIDSSRIPSFTQLHSTMTRAPLLLLCVALVLLGH | Core Human ORF Only |
| | 4419 | RFS | | | VNGATVRNEDKWKPLNNPRNRDLFFRRLQAYFKG | |
| | | | | | RGLDLGTFPNPFPTNENPRPLSFQSELTASASAD | |
| | | | | | YEEQKNSFHNYLKG | |
| 83 | JHU1 | IPL | 0.099215254 | SCHIP | MSPILGYWKIKGLVQPTRLLLEYLEEKYEEHLYER | Full Literal Sequence- |
| | 5263 | RFS | | 1 | DEGDKWRNKKFELGLEFPNLPYYIDGDVKLTQSMA | (including GST-tag, 6- |
| | | | | | IIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAFP | ORF, occasional ‘post |
| | | | | | KLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVHQDNCSYQAQKN | |
| | | | | | ERESIRQKLALGSFFDDGPGIYTSCSKSGKPSLS | |
| | | | | | SRLQSGMNLQICFVNDSGSDKDSDADDSKTETSL | |
| | | | | | DTPLSPMSKQSSSYSDRDTTEEESESLDDMDFLT | |
| | | | | | RQKKLQAEAKMALAMAKPMAKMQVEVEKQNRK | |
| | | | | | KSPVADLLPHMPHISECLMKRSLKPTDLRDMTIG | |
| | | | | | QLQVIVNDLHSQIESLNEELVQLLLIRDELHTEQ | |
| | | | | | DAMLVDIEDLTRHAESQQKHMVEKMPAK | |
| 84 | JHU1 | IPL | 0.099215254 | SCHIP | MVHQDNCSYQAQKNERESIRQKLALGSFFDDGP | Core Human ORF Only |
| | 5263 | RFS | | 1 | GIYTSCSKSGKPSLSSRLQSGMNLQICFVNDSGS | |
| | | | | | DKDSDADDSKTETSLDTPLSPMSKQSSSYSDRD | |
| | | | | | TTEEESESLDDMDFLTRQKKLQAEAKMALAMAKP | |
| | | | | | MAKMQVEVEKQNRKKSPVADLLPHMPHISECLM | |
| | | | | | KRSLKPTDLRDMTIGQLQVIVNDLHSQIESLNEEL | |
| | | | | | VQLLLIRDELHTEQDAMLVDIEDLTRHAESQQKH | |
| | | | | | MVEKMPAK | |
| 85 | JHU1 | IPL | 0.114945062 | BEND6 | MSPILGYWKIKGLVQPTRLLLEYLEEKYEEHLYER | Full Literal Sequence- |
| | 5591 | RFS | | | DEGDKWRNKKFELGLEFPNLPYYIDGDVKLTQSMA | (including GST-tag, 6- |
| | | | | | IIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMQKIVQTDEITNTQA | |
| | | | | | FRKGKRKRTETMDSENANSDMDKGQRDPYSGN | |
| | | | | | AFLPGESSSEDEEPLAELSKEELCAKIKSLKEKLT | |
| | | | | | NTRKENSRLRQSLVMLQAWSRELP | |
| 86 | JHU1 | IPL | 0.114945062 | BEND6 | MQKIVQTDEITNTQAFRKGKRKRTETMDSENANS | Core Human ORF Only |
| | 5591 | RFS | | | DMDKGQRDPYSGNAFLPGESSSEDEEPLAELSK | |
| | | | | | EELCAKIKSLKEKLTNTRKENSRLRQSLVMLQAW | |
| | | | | | SRELP | |
| 87 | JHU1 | IPL | 0.357849606 | MMP3 | MSPILGYWKIKGLVQPTRLLLEYLEEKYEEHLYER | Full Literal Sequence- |
| | 5629 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKSLPILLLLCVAVCS | |
| | | | | | AYPLDGAARGEDTSMNLVQKYLENYYDLEKDVK | |
| | | | | | QFVRRKDSGPVVKKIREMQKFLGLEVTGKLDSDT | |
| | | | | | LEVMRKPRCGVPDVGHFRTFPGIPKWRKTHLTY | |
| | | | | | RIVNYTPDLPKDAVDSAVEKALKVWEEVTPLTFS | |
| | | | | | RLYEGEADIMISFAVREHGDFYPFDGPGNVLAHA | |
| | | | | | YAPGPGINGDAHFDDDEQWTKDTTGTNLFLVAA | |
| | | | | | HEIGHSLGLFHSANTEALMYPLYHSLTDLTRFRLS | |
| | | | | | QDDINGIQSLYGPPPDSPETPLVPTEPVPPEPGTP | |
| | | | | | ANCDPALSFDAVSTLRGEILIFKDRHFWRKSLRKL | |
| | | | | | EPELHLISSFWPSLPSGVDAAYEVTSKDLVFIFKG | |
| | | | | | NQFWAIRGNEVRAGYPRGIHTLGFPPTVRKIDAAI | |
| | | | | | SDKEKNKTYFFVEDKYWRFDEKRNSMEPGFPKQI | |
| | | | | | AEDFPGIDSKIDAVFEEFGFFYFFTGSSQLEFDPN | |
| | | | | | AKKVTHTLKSNSWLNC | |
| 88 | JHU1 | IPL | 0.357849606 | MMP3 | MKSLPILLLLCVAVCSAYPLDGAARGEDTSMNLV | Core Human ORF Only |
| | 5629 | RFS | | | QKYLENYYDLEKDVKQFVRRKDSGPVVKKIREM | |
| | | | | | QKFLGLEVTGKLDSDTLEVMRKPRCGVPDVGHF | |
| | | | | | RTFPGIPKWRKTHLTYRIVNYTPDLPKDAVDSAVE | |
| | | | | | KALKVWEEVTPLTFSRLYEGEADIMISFAVREHGD | |
| | | | | | FYPFDGPGNVLAHAYAPGPGINGDAHFDDDEQW | |
| | | | | | TKDTTGTNLFLVAAHEIGHSLGLFHSANTEALMYP | |
| | | | | | LYHSLTDLTRFRLSQDDINGIQSLYGPPPDSPETP | |
| | | | | | LVPTEPVPPEPGTPANCDPALSFDAVSTLRGEILI | |
| | | | | | FKDRHFWRKSLRKLEPELHLISSFWPSLPSGVDA | |
| | | | | | AYEVTSKDLVFIFKGNQFWAIRGNEVRAGYPRGI | |
| | | | | | HTLGFPPTVRKIDAAISDKEKNKTYFFVEDKYWRF | |
| | | | | | DEKRNSMEPGFPKQIAEDFPGIDSKIDAVFEEFGF | |
| | | | | | FYFFTGSSQLEFDPNAKKVTHTLKSNSWLNC | |
| 89 | JHU1 | IPL | 0.254084819 | PAK2 | MSPILGYWKIKGLVQPTRLLLEYLEEKYEEHLYER | Full Literal Sequence- |
| | 5639 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSDNGELEDKPPAP | |
| | | | | | PVRMSSTIFSTGGKDPLSANHSLKPLPSVPEEKK | |
| | | | | | PRHKIISIFSGTEKGSKKKEKERPEISPPSDFEHTI | |
| | | | | | HVGFDAVTGEFTGMPEQWARLLQTSNITKLEQKK | |
| | | | | | NPQAVLDVLKFYDSNTVKQKYLSFTPPEKDGFPS | |
| | | | | | GTPALNAKGTEAPAVVTEEEDDDEETAPPVIAPR | |
| | | | | | PDHTKSIYTRSVIDPVPAPVGDSHVDGAAKSLDK | |
| | | | | | QKKKTKMTDEEIMEKLRTIVSIGDPKKKYTRYEKI | |
| | | | | | GQRASGTVFTATDVALGQEVAIKQINLQKQPKKE | |
| | | | | | LIINEILVMKELKNPNIVNFLDSYLVGDELFVVMEY | |
| | | | | | LAGGSLTDVVTETCMDEAQIAAVCRECLQALEFL | |
| | | | | | HANQVIHRDIKSDNVLLGMEGSVKLTDFGFCAQIT | |
| | | | | | PEQSKRSTMVGTPYWMAPEVVTRKAYGPKVDIW | |
| | | | | | SLGIMAIEMVEGEPPYLNENPLRALYLIATNGTPEL | |
| | | | | | QNPEKLSPIFRDFLNRCLEMDVEKRGSAKELLQH | |
| | | | | | PFLKLAKPLSSLTPLIMAAKEAMKSNR | |
| 90 | JHU1 | IPL | 0.254084819 | PAK2 | MSDNGELEDKPPAPPVRMSSTIFSTGGKDPLSAN | Core Human ORF Only |
| | 5639 | RFS | | | HSLKPLPSVPEEKKPRHKIISIFSGTEKGSKKKEKE | |
| | | | | | RPEISPPSDFEHTIHVGFDAVTGEFTGMPEQWAR | |
| | | | | | LLQTSNITKLEQKKNPQAVLDVLKFYDSNTVKQKY | |
| | | | | | LSFTPPEKDGFPSGTPALNAKGTEAPAVVTEEED | |
| | | | | | DDEETAPPVIAPRPDHTKSIYTRSVIDPVPAPVGD | |
| | | | | | SHVDGAAKSLDKQKKKTKMTDEEIMEKLRTIVSIG | |
| | | | | | DPKKKYTRYEKIGQRASGTVFTATDVALGQEVAIK | |
| | | | | | QINLQKQPKKELIINEILVMKELKNPNIVNFLDSYL | |
| | | | | | VGDELFVVMEYLAGGSLTDVVTETCMDEAQIAAVC | |
| | | | | | RECLQALEFLHANQVIHRDIKSDNVLLGMEGSVKL | |
| | | | | | TDFGFCAQITPEQSKRSTMVGTPYWMAPEVVTR | |
| | | | | | KAYGPKVDIWSLGIMAIEMVEGEPPYLNENPLRAL | |
| | | | | | YLIATNGTPELQNPEKLSPIFRDFLNRCLEMDVEK | |
| | | | | | RGSAKELLQHPFLKLAKPLSSLTPLIMAAKEAMKS | |
| | | | | | NR | |
| 91 | JHU1 | IPL | 0.311170063 | RRM1 | MSPILGYWKIKGLVQPTRLLLEYLEEKYEEHLYER | Full Literal Sequence- |
| | 5652 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMHVIKRDGRQERVM | |
| | | | | | FDKITSRIQKLCYGLNMDFVDPAQITMKVIQGLYS | |
| | | | | | GVTTVELDTLAAETAATLTTKHPDYAILAARIAVSN | |
| | | | | | LHKETKKVFSDVMEDLYNYINPHNGKHSPMVAKS | |
| | | | | | TLDIVLANKDRLNSAIIYDRDFSYNYFGFKTLERSY | |
| | | | | | LLKINGKVAERPQHMLMRVSVGIHKEDIDAAIETY | |
| | | | | | NLLSERWFTHASPTLFNAGTNRPQLSSCFLLSMK | |
| | | | | | DDSIEGIYDTLKQCALISKSAGGIGVAVSCIRATGS | |
| | | | | | YIAGTNGNSNGLVPMLRVYNNTARYVDQGGNKR | |
| | | | | | PGAFAIYLEPWHLDIFEFLDLKKNTGKEEQRARDL | |
| | | | | | FFALWIPDLFMKRVETNQDWSLMCPNECPGLDE | |
| | | | | | VWGEEFEKLYASYEKQGRVRKVVKAQQLWYAIIE | |
| | | | | | SQTETGTPYMLYKDSCNRKSNQQNLGTIKCSNLC | |
| | | | | | TEIVEYTSKDEVAVCNLASLALNMYVTSEHTYDFK | |
| | | | | | KLAEVTKVVVRNLNKIIDINYYPVPEACLSNKRHR | |
| | | | | | PIGIGVQGLADAFILMRYPFESAEAQLLNKQIFETI | |
| | | | | | YYGALEASCDLAKEQGPYETYEGSPVSKGILQYD | |
| | | | | | MWNVTPTDLWDWKVLKEKIAKYGIRNSLLIAPMP | |
| | | | | | TASTAQILGNNESIEPYTSNIYTRRVLSGEFQIVNP | |
| | | | | | HLLKDLTERGLWHEEMKNQIIACNGSIQSIPEIPDD | |
| | | | | | LKQLYKTVWEISQKTVLKMAAERGAFIDQSQSLNI | |
| | | | | | HIAEPNYGKLTSMHFYGWKQGLKTGMYYLRTRP | |
| | | | | | AANPIQFTLNKEKLKDKEKVSKEEEEKERNTAAM | |
| | | | | | VCSLENRDECLMCGS | |
| 92 | JHU1 | IPL_ | 0.311170063 | RRM1 | MHVIKRDGRQERVMFDKITSRIQKLCYGLNMDFV | Core Human ORF Only |
| | 5652 | RFS | | | DPAQITMKVIQGLYSGVTTVELDTLAAETAATLTT | |
| | | | | | KHPDYAILAARIAVSNLHKETKKVFSDVMEDLYNY | |
| | | | | | INPHNGKHSPMVAKSTLDIVLANKDRLNSAIIYDR | |
| | | | | | DFSYNYFGFKTLERSYLLKINGKVAERPQHMLMR | |
| | | | | | VSVGIHKEDIDAAIETYNLLSERWFTHASPTLFNA | |
| | | | | | GTNRPQLSSCFLLSMKDDSIEGIYDTLKQCALISK | |
| | | | | | SAGGIGVAVSCIRATGSYIAGTNGNSNGLVPMLR | |
| | | | | | VYNNTARYVDQGGNKRPGAFAIYLEPWHLDIFEF | |
| | | | | | LDLKKNTGKEEQRARDLFFALWIPDLFMKRVETN | |
| | | | | | QDWSLMCPNECPGLDEVWGEEFEKLYASYEKQ | |
| | | | | | GRVRKVVKAQQLWYAIIESQTETGTPYMLYKDSC | |
| | | | | | NRKSNQQNLGTIKCSNLCTEIVEYTSKDEVAVCNL | |
| | | | | | ASLALNMYVTSEHTYDFKKLAEVTKVVVRNLNKII | |
| | | | | | DINYYPVPEACLSNKRHRPIGIGVQGLADAFILMR | |
| | | | | | YPFESAEAQLLNKQIFETIYYGALEASCDLAKEQG | |
| | | | | | PYETYEGSPVSKGILQYDMWNVTPTDLWDWKVLKE | |
| | | | | | KIAKYGIRNSLLIAPMPTASTAQILGNNESIEPYT | |
| | | | | | SNIYTRRVLSGEFQIVNPHLLKDLTERGLWHEEMK | |
| | | | | | NQIIACNGSIQSIPEIPDDLKQLYKTVWEISQKTV | |
| | | | | | LKMAAERGAFIDQSQSLNIHIAEPNYGKLTSMHFY | |
| | | | | | GWKQGLKTGMYYLRTRPAANPIQFTLNKEKLKDK | |
| | | | | | EKVSKEEEEKERNTAAMVCSLENRDECLMCGS | |
| 93 | JHU1 | IPI_ | 0.106424211 | PLEKH | MSPILGYWKIKGLVQPTRLLLEYLEEKYEEHLYER | Full Literal Sequence- |
| | 5734 | RFS | | G6 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKAFGPPHEGPLQG | |
| | | | | | LVASRIETYGGRHRASAQSTTGRLYPRGYPVLDP | |
| | | | | | SRRRLQQYVPFARGSGQARGLSPMRLRDPEPEK | |
| | | | | | RHGGHVGAGLLHSPKLKELTKAHELEVRLHTFSM | |
| | | | | | FGMPRLPPEDRRHWEIGEGGDSGLTIEKSWREL | |
| | | | | | VPGHKEMSQELCHQQEALWELLTTELIYVRKLKI | |
| | | | | | MTDLLAAGLLNLQRVGLLMEVSAETLFGNVPSLIR | |
| | | | | | THRSFWDEVLGPTLEETRASGQPLDPIGLQSGFL | |
| | | | | | TFGQRFHPYVQYCLRVKQTMAYAREQQETNPLF | |
| | | | | | HAFVQWCEKHKRSGRQMLCDLLIKPHQRITKYPL | |
| | | | | | LLHAVLKRSPEARAQEALNAMIEAVESFLRHINGQ | |
| | | | | | VRQGEEQESLAAAAQRIGPYEVLEPPSDEVGKNL | |
| | | | | | RPFSTLDLTSPMLGVASEHTRQLLLEGPVRVKEG | |
| | | | | | REGKLDVYLFLFSDVLLVTKPQRKADKAKVIRPPL | |
| | | | | | MLEKLVCQPLRDPNSFLLIHLTEFQCVSSALLVHC | |
| | | | | | PSPTDRAQWLEKTQQAQAALQKLKAEEYVQQKR | |
| | | | | | ELLTLYRDQDRESPSTRPSTPSLEGSQSSAEGRT | |
| | | | | | PEFSTIIPHLVVTEDTDEDAPLVPDDTSDSGYGTLI | |
| | | | | | PGTPTGSRSPLSRLRQRALRRDPRLTFSTLELRDI | |
| | | | | | PLRPHPPDPQAPQRRSAPELPEGILKGGSLPQED | |
| | | | | | PPTWSEEEDGASERGNVVVETLHRARLRGQLPS | |
| | | | | | SPTHADSAGESPWESSGEEEEEGPLFLKAGHTS | |
| | | | | | LRPMRAEDMLREIREELASQRIEGAEEPRDSRPR | |
| | | | | | KLTRAQLQRMRGPHIIQLDTPLSASEV | |
| 94 | JHU1 | IPI_ | 0.106424211 | PLEKH | MKAFGPPHEGPLQGLVASRIETYGGRHRASAQS | Core Human ORF Only |
| | 5734 | RFS | | G6 | TTGRLYPRGYPVLDPSRRRLQQYVPFARGSGQA | |
| | | | | | RGLSPMRLRDPEPEKRHGGHVGAGLLHSPKLKE | |
| | | | | | LTKAHELEVRLHTFSMFGMPRLPPEDRRHWEIGE | |
| | | | | | GGDSGLTIEKSWRELVPGHKEMSQELCHQQEAL | |
| | | | | | WELLTTELIYVRKLKIMTDLLAAGLLNLQRVGLLM | |
| | | | | | EVSAETLFGNVPSLIRTHRSFWDEVLGPTLEETR | |
| | | | | | ASGQPLDPIGLQSGFLTFGQRFHPYVQYCLRVKQ | |
| | | | | | TMAYAREQQETNPLFHAFVQWCEKHKRSGRQM | |
| | | | | | LCDLLIKPHQRITKYPLLLHAVLKRSPEARAQEALN | |
| | | | | | AMIEAVESFLRHINGQVRQGEEQESLAAAAQRIG | |
| | | | | | PYEVLEPPSDEVGKNLRPFSTLDLTSPMLGVASE | |
| | | | | | HTRQLLLEGPVRVKEGREGKLDVYLFLFSDVLLV | |
| | | | | | TKPQRKADKAKVIRPPLMLEKLVCQPLRDPNSFLL | |
| | | | | | IHLTEFQCVSSALLVHCPSPTDRAQWLEKTQQAQ | |
| | | | | | AALQKLKAEEYVQQKRELLTLYRDQDRESPSTRP | |
| | | | | | STPSLEGSQSSAEGRTPEFSTIIPHLVVTEDTDED | |
| | | | | | APLVPDDTSDSGYGTLIPGTPTGSRSPLSRLRQR | |
| | | | | | ALRRDPRLTFSTLELRDIPLRPHPPDPQAPQRRS | |
| | | | | | APELPEGILKGGSLPQEDPPTWSEEEDGASERG | |
| | | | | | NVVVETLHRARLRGQLPSSPTHADSAGESPWES | |
| | | | | | SGEEEEEGPLFLKAGHTSLRPMRAEDMLREIREE | |
| | | | | | LASQRIEGAEEPRDSRPRKLTRAQLQRMRGPHII | |
| | | | | | QLDTPLSASEV | |
| 95 | JHU1 | IPL_ | 0.131964772 | PRMT8 | MSPILGYWKIKGLVQPTRLLLEYLEEKYEEHLYER | Full Literal Sequence- |
| | 6550 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGMKHSSRCLLLRR | |
| | | | | | KMAENAAESTEVNSPPSQPPQPVVPAKPVQCVH | |
| | | | | | HVSTQPSCPGRGKMSKLLNPEEMTSRDYYFDSY | |
| | | | | | AHFGIHEEMLKDEVRTLTYRNSMYHNKHVFKDKV | |
| | | | | | VLDVGSGTGILSMFAAKAGAKKVFGIECSSISDYS | |
| | | | | | EKIIKANHLDNIITIFKGKVEEVELPVEKVDIIISE | |
| | | | | | WMGYCLFYESMLNTVIFARDKWLKPGGLMFPDRAAL | |
| | | | | | YVVAIEDRQYKDFKIHVWVENVYGFDMTCIRDVAM | |
| | | | | | KEPLVDIVDPKQVVTNACLIKEVDIYTVKTEELSFT | |
| | | | | | SAFCLQIQRNDYVHALVTYFNIEFTKCHKKMGFST | |
| | | | | | APDAPYTHWKQTVFYLEDYLTVRRGEEIYGTISM | |
| | | | | | KPNAKNVRDLDFTVDLDFKGQLCETSVSNDYKM | |
| | | | | | R | |
| 96 | JHU1 | IPL | 0.131964772 | PRMT8 | MGMKHSSRCLLLRRKMAENAAESTEVNSPPSQP | Core Human ORF Only |
| | 6550 | RFS | | | PQPVVPAKPVQCVHHVSTQPSCPGRGKMSKLLN | |
| | | | | | PEEMTSRDYYFDSYAHFGIHEEMLKDEVRTLTYR | |
| | | | | | NSMYHNKHVFKDKVVLDVGSGTGILSMFAAKAGAK | |
| | | | | | KVFGIECSSISDYSEKIIKANHLDNIITIFKGKVEE | |
| | | | | | VELPVEKVDIIISEWMGYCLFYESMLNTVIFARDK | |
| | | | | | WLKPGGLMFPDRAALYVVAIEDRQYKDFKIHVWV | |
| | | | | | ENVYGFDMTCIRDVAMKEPLVDIVDPKQVVTNAC | |
| | | | | | LIKEVDIYTVKTEELSFTSAFCLQIQRNDYVHALVT | |
| | | | | | YFNIEFTKCHKKMGFSTAPDAPYTHWKQTVFYLE | |
| | | | | | DYLTVRRGEEIYGTISMKPNAKNVRDLDFTVDLDF | |
| | | | | | KGQLCETSVSNDYKMR | |
| 97 | JHU1 | IPL | 0.38316599 | RBAK | MSPILGYWKIKGLVQPTRLLLEYLEEKYEEHLYER | Full Literal Sequence- |
| | 6766 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNTLQGPVSFKDVA | |
| | | | | | VDFTQEEWQQLDPDEKITYRDVMLENYSHLVSV | |
| | | | | | GYDTTKPNVIIKLEQGEEPWIMGGEFPCQHSPEA | |
| | | | | | WRVDDLIERIQENEDKHSRQAACINSKTLTEEKEN | |
| | | | | | TFSQIYMETSLVPSSIIAHNCVSCGKNLESISOLIS | |
| | | | | | SDGSYARTKPDECNECGKTYHGEKMCEFNQNG | |
| | | | | | DTYSHNEENILQKISILEKPFEYNECMEALDNEAV | |
| | | | | | FIAHKRAYIGEKPYEWNDSGPDFIQMSNFNAYQR | |
| | | | | | SQMEMKPFECSECGKSFCKKSKFIIHQRAHTGEK | |
| | | | | | PYECNVCGKSFSQKGTLTVHRRSHLEEKPYKCN | |
| | | | | | ECGKTFCQKLHLTQHLRTHSGEKPYECSECGKT | |
| | | | | | FCQKTHLTLHQRNHSGERPYPCNECGKSFSRKS | |
| | | | | | ALSDHQRTHTGEKLYKCNECGKSYYRKSTLITHQ | |
| | | | | | RTHTGEKPYQCSECGKFFSRVSYLTIHYRSHLEE | |
| | | | | | KPYECNECGKTFNLNSAFIRHRKVHTEEKSHECS | |
| | | | | | ECGKFSQLYLTDHHTAHLEEKPYECNECGKTFLV | |
| | | | | | NSAFDGHQPLPKGEKSYECNVCGKLFNELSYYT | |
| | | | | | EHYRSHSEEKPYGCSECGKTFSHNSSLFRHQRV | |
| | | | | | HTGEKPYECYECGKFFSQKSYLTIHHRIHSGEKP | |
| | | | | | YECSKCGKVFSRMSNLTVHYRSHSGEKPYECNE | |
| | | | | | CGKVFSQKSYLTVHYRTHSGEKPYECNECGKKF | |
| | | | | | HHRSAFNSHQRIHRRGNMNVLDVENL | |
| 98 | JHU1 | IPL | 0.38316599 | RBAK | MNTLQGPVSFKDVAVDFTQEEWQQLDPDEKITY | Core Human ORF Only |
| | 6766 | RFS | | | RDVMLENYSHLVSVGYDTTKPNVIIKLEQGEEPWI | |
| | | | | | MGGEFPCQHSPEAWRVDDLIERIQENEDKHSRQ | |
| | | | | | AACINSKTLTEEKENTFSQIYMETSLVPSSIIAHNC | |
| | | | | | VSCGKNLESISQLISSDGSYARTKPDECNECGKT | |
| | | | | | YHGEKMCEFNQNGDTYSHNEENILQKISILEKPFE | |
| | | | | | YNECMEALDNEAVFIAHKRAYIGEKPYEWNDSGP | |
| | | | | | DFIQMSNFNAYQRSQMEMKPFECSECGKSFCKK | |
| | | | | | SKFIIHQRAHTGEKPYECNVCGKSFSQKGTLTVH | |
| | | | | | RRSHLEEKPYKCNECGKTFCQKLHLTQHLRTHS | |
| | | | | | GEKPYECSECGKTFCQKTHLTLHQRNHSGERPY | |
| | | | | | PCNECGKSFSRKSALSDHQRTHTGEKLYKCNEC | |
| | | | | | GKSYYRKSTLITHQRTHTGEKPYQCSECGKFFSR | |
| | | | | | VSYLTIHYRSHLEEKPYECNECGKTFNLNSAFIRH | |
| | | | | | RKVHTEEKSHECSECGKFSQLYLTDHHTAHLEEK | |
| | | | | | PYECNECGKTFLVNSAFDGHQPLPKGEKSYECN | |
| | | | | | VCGKLFNELSYYTEHYRSHSEEKPYGCSECGKTF | |
| | | | | | SHNSSLFRHQRVHTGEKPYECYECGKFFSQKSY | |
| | | | | | LTIHHRIHSGEKPYECSKCGKVFSRMSNLTVHYR | |
| | | | | | SHSGEKPYECNECGKVFSQKSYLTVHYRTHSGE | |
| | | | | | KPYECNECGKKFHHRSAFNSHQRIHRRGNMNVL | |
| | | | | | DVENL | |
| 99 | JHU1 | IPL_ | 0.177302642 | UGT2B | MSPILGYWKIKGLVQPTRLLLEYLEEKYEEHLYER | Full Literal Sequence- |
| | 7501 | RFS | | 15 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSLKWTSVFLLIQLS | |
| | | | | | CYFSSGSCGKVLVWPTEYSHWINMKTILEELVQR | |
| | | | | | GHEVTVLTSSASTLVNASKSSAIKLEVYPTSLTKN | |
| | | | | | YLEDSLLKILDRWIYGVSKNTFWSYFSQLQELCW | |
| | | | | | EYYDYSNKLCKDAVLNKKLMMKLQESKFDVILAD | |
| | | | | | ALNPCGELLAELFNIPFLYSLRFSVGYTFEKNGGG | |
| | | | | | FLFPPSYVPVVMSELSDQMIFMERIKNMIHMLYFD | |
| | | | | | FWFQIYDLKKWDQFYSEVLGRPTTLFETMGKAE | |
| | | | | | MWLIRTYWDFEFPRPFLPNVDFVGGLHCKPAKPL | |
| | | | | | PKEMEEFVQSSGENGIVVFSLGSMISNMSEESAN | |
| | | | | | MIASALAQIPQKVLWRFDGKKPNTLGSNTRLYKW | |
| | | | | | LPQNDLLGHPKTKAFITHGGTNGIYEAIYHGIPMV | |
| | | | | | GIPLFADQHDNIAHMKAKGAALSVDIRTMSSRDLL | |
| | | | | | NALKSVINDPVYKENVMKLSRIHHDQPMKPLDRA | |
| | | | | | VFWIEFVMRHKGAKHLRVAAHNLTWIQYHSLDVI | |
| | | | | | AFLLACVATVIFIITKFCLFCFRKLAKKGKKKKR | |
| | | | | | DNPAFLYKVVKASS | |
| 100 | JHU1 | IPL | 0.177302642 | UGT2B | MSLKWTSVFLLIQLSCYFSSGSCGKVLVWPTEYS | Core Human ORF Only |
| | 7501 | RFS | | 15 | HWINMKTILEELVQRGHEVTVLTSSASTLVNASKS | |
| | | | | | SAIKLEVYPTSLTKNYLEDSLLKILDRWIYGVSKNT | |
| | | | | | FWSYFSQLQELCWEYYDYSNKLCKDAVLNKKLM | |
| | | | | | MKLQESKFDVILADALNPCGELLAELFNIPFLYSLR | |
| | | | | | FSVGYTFEKNGGGFLFPPSYVPVVMSELSDQMIF | |
| | | | | | MERIKNMIHMLYFDFWFQIYDLKKWDQFYSEVLG | |
| | | | | | RPTTLFETMGKAEMWLIRTYWDFEFPRPFLPNVD | |
| | | | | | FVGGLHCKPAKPLPKEMEEFVQSSGENGIVVFSL | |
| | | | | | GSMISNMSEESANMIASALAQIPQKVLWRFDGKK | |
| | | | | | PNTLGSNTRLYKWLPQNDLLGHPKTKAFITHGGT | |
| | | | | | NGIYEAIYHGIPMVGIPLFADQHDNIAHMKAKGAA | |
| | | | | | LSVDIRTMSSRDLLNALKSVINDPVYKENVMKLSR | |
| | | | | | IHHDQPMKPLDRAVFWIEFVMRHKGAKHLRVAAH | |
| | | | | | NLTWIQYHSLDVIAFLLACVATVIFIITKFCLFCF | |
| | | | | | RKLAKKGKKKKRD | |
| 101 | JHU1 | IPL | 0.158315773 | RAET1 | MSPILGYWKIKGLVQPTRLLLEYLEEKYEEHLYER | Full Literal Sequence- |
| | 7574 | RFS | | L | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAAAIPALLLCLPLL | |
| | | | | | FLLFGWSRARRDDPHSLCYDITVIPKFRPGPRWC | |
| | | | | | AVQGQVDEKTFLHYDCGNKTVTPVSPLGKKLNVT | |
| | | | | | MAWKAQNPVLREVVDILTEQLLDIQLENYTPKEPL | |
| | | | | | TLQARMSCEQKAEGHSSGSWQFSIDGQTFLLFD | |
| | | | | | SEKRMWTTVHPGARKMKEKWENDKDVAMSFHY | |
| | | | | | ISMGDCIGWLEDFLMGMDSTLEPSAGAPLAMSS | |
| | | | | | GTTQLRATATTLILCCLLIILPCFILPGINPAF | |
| | | | | | LYKVVKASS | |
| 102 | JHU1 | IPL | 0.158315773 | RAET1 | MAAAAIPALLLCLPLLFLLFGWSRARRDDPHSLCY | Core Human ORF Only |
| | 7574 | RFS | | L | DITVIPKFRPGPRWCAVQGQVDEKTFLHYDCGNK | |
| | | | | | TVTPVSPLGKKLNVTMAWKAQNPVLREVVDILTE | |
| | | | | | QLLDIQLENYTPKEPLTLQARMSCEQKAEGHSSG | |
| | | | | | SWQFSIDGQTFLLFDSEKRMWTTVHPGARKMKE | |
| | | | | | KWENDKDVAMSFHYISMGDCIGWLEDFLMGMDS | |
| | | | | | TLEPSAGAPLAMSSGTTQLRATATTLILCCLLIILP | |
| | | | | | CFILPGI | |
| 103 | JHU1 | IPL_ | 0.17910634 | OGDH | MSPILGYWKIKGLVQPTRLLLEYLEEKYEEHLYER | Full Literal Sequence- |
| | 8208 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMFHLRTCAAKLRPLT | |
| | | | | | ASQTVKTFSQNRPAAARTFQQIRCYSAPVAAEPF | |
| | | | | | LSGTSSNYVEEMYCAWLENPKSVHKSWDIFFRN | |
| | | | | | TNAGAPPGTAYQSPLPLSRGSLAAVAHAQSLVEA | |
| | | | | | QPNVDKLVEDHLAVQSLIRAYQIRGHHVAQLDPL | |
| | | | | | GILDADLDSSVPADIISSTDKLGFYGLDESDLDKVF | |
| | | | | | HLPTTTFIGGQESALPLREIIRRLEMAYCQHIGVEF | |
| | | | | | MFINDLEQCQWIRQKFETPGIMQFTNEEKRTLLA | |
| | | | | | RLVRSTRFEEFLQRKWSSEKRFGLEGCEVLIPAL | |
| | | | | | KTIIDKSSENGVDYVIMGMPHRGRLNVLANVIRKE | |
| | | | | | LEQIFCQFDSKLEAADEGSGDVKYHLGMYHRRIN | |
| | | | | | RVTDRNITLSLVANPSHLEAADPVVMGKTKAEQF | |
| | | | | | YCGDTEGKKVMSILLHGDAAFAGQGIVYETFHLS | |
| | | | | | DLPSYTTHGTVHVVVNNQIGFTTDPRMARSSPYP | |
| | | | | | TDVARVVNAPIFHVNSDDPEAVMYVCKVAAEWR | |
| | | | | | STFHKDVVVDLVCYRRNGHNEMDEPMFTQPLMY | |
| | | | | | KQIRKQKPVLQKYAELLVSQGVVNQPEYEEEISK | |
| | | | | | YDKICEEAFARSKDEKILHIKHWLDSPWPGFFTLD | |
| | | | | | GQPRSMSCPSTGLTEDILTHIGNVASSVPVENFTI | |
| | | | | | HGGLSRILKTRGEMVKNRTVDWALAEYMAFGSLL | |
| | | | | | KEGIHIRLSGQDVERGTFSHRHHVLHDQNVDKRT | |
| | | | | | CIPMNHLWPNQAPYTVCNSSLSEYGVLGFELGFA | |
| | | | | | MASPNALVLWEAQFGDFHNTAQCIIDQFICPGQA | |
| | | | | | KVWRQNGIVLLLPHGMEGMGPEHSSARPERFLQ | |
| | | | | | MCNDDPDVLPDLKEANFDINQLYDCNWVVVNCS | |
| | | | | | TPGNFFHVLRRQILLPFRKPLIIFTPKSLLRHPEAR | |
| | | | | | SSFDEMLPGTHFQRVIPEDGPAAQNPENVKRLLF | |
| | | | | | CTGKVYYDLTRERKARDMVGQVAITRIEQLSPFP | |
| | | | | | FDLLLKEVQKYPNAELAWCQEEHKNQGYYDYVK | |
| | | | | | PRLRTTISRAKPVWYAGRDPAAAPATGNKKTHLT | |
| | | | | | ELQRLLDTAFDLDVFKNFSNPAFLYKVVKASS | |
| 104 | JHU1 | IPI_ | 0.1791063 | OGDH | MFHLRTCAAKLRPLTASQTVKTFSQNRPAAARTF | Core Human ORF Only |
| | 8208 | RFS | 4 | | QQIRCYSAPVAAEPFLSGTSSNYVEEMYCAWLE | |
| | | | | | NPKSVHKSWDIFFRNTNAGAPPGTAYQSPLPLSR | |
| | | | | | GSLAAVAHAQSLVEAQPNVDKLVEDHLAVQSLIR | |
| | | | | | AYQIRGHHVAQLDPLGILDADLDSSVPADIISSTDK | |
| | | | | | LGFYGLDESDLDKVFHLPTTTFIGGQESALPLREII | |
| | | | | | RRLEMAYCQHIGVEFMFINDLEQCQWIRQKFETP | |
| | | | | | GIMQFTNEEKRTLLARLVRSTRFEEFLQRKWSSE | |
| | | | | | KRFGLEGCEVLIPALKTIIDKSSENGVDYVIMGMP | |
| | | | | | HRGRLNVLANVIRKELEQIFCQFDSKLEAADEGS | |
| | | | | | GDVKYHLGMYHRRINRVTDRNITLSLVANPSHLE | |
| | | | | | AADPVVMGKTKAEQFYCGDTEGKKVMSILLHGD | |
| | | | | | AAFAGQGIVYETFHLSDLPSYTTHGTVHVVVNNQI | |
| | | | | | GFTTDPRMARSSPYPTDVARVVNAPIFHVNSDDP | |
| | | | | | EAVMYVCKVAAEWRSTFHKDVVVDLVCYRRNGH | |
| | | | | | NEMDEPMFTQPLMYKQIRKQKPVLQKYAELLVSQ | |
| | | | | | GVVNQPEYEEEISKYDKICEEAFARSKDEKILHIKH | |
| | | | | | WLDSPWPGFFTLDGQPRSMSCPSTGLTEDILTHI | |
| | | | | | GNVASSVPVENFTIHGGLSRILKTRGEMVKNRTV | |
| | | | | | DWALAEYMAFGSLLKEGIHIRLSGQDVERGTFSH | |
| | | | | | RHHVLHDQNVDKRTCIPMNHLWPNQAPYTVCNS | |
| | | | | | SLSEYGVLGFELGFAMASPNALVLWEAQFGDFH | |
| | | | | | NTAQCIIDQFICPGQAKVWRQNGIVLLLPHGMEG | |
| | | | | | MGPEHSSARPERFLQMCNDDPDVLPDLKEANFD | |
| | | | | | INQLYDCNVWVVNCSTPGNFFHVLRRQILLPFRK | |
| | | | | | PLIIFTPKSLLRHPEARSSFDEMLPGTHFQRVIPED | |
| | | | | | GPAAQNPENVKRLLFCTGKVYYDLTRERKARDM | |
| | | | | | VGQVAITRIEQLSPFPFDLLLKEVQKYPNAELAWC | |
| | | | | | QEEHKNQGYYDYVKPRLRTTISRAKPVWYAGRD | |
| | | | | | PAAAPATGNKKTHLTELQRLLDTAFDLDVFKNFS | |
| 105 | JHU2 | IPI_ | 0.037500193 | HSFX1 | MSPILGYWKIKGLVQPTRLLLEYLEEKYEEHLYER | Full Literal Sequence- |
| | 0559 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEDKRSLSMARCEE | |
| | | | | | RNSRGQDHGLERVPFPPQLQSETYLHPADPSPA | |
| | | | | | WDDPGSTGSPNLRLLTEEIAFQPLAEEASFRRPH | |
| | | | | | PDGDVPPQGEDNLLSLPFPQKLWRLVSSNQFSSI | |
| | | | | | VWVDDSGACRVINQKLFEKEILKRDVAHKVFATTSI | |
| | | | | | KSFFRQLNLYGFRKRRQCTFRTFTRIFSAKRLVSI | |
| | | | | | LNKLEFYCHPYFQRDSPHLLVRMKRRVGVKSAP | |
| | | | | | RHQEEDKPEAAGSCLAPADTEQQDHTSPNENDQ | |
| | | | | | VTPQHREPAGPNTQIRSGSAPPATPVMVPDSAV | |
| | | | | | ASDNSPVTQPAGEWSEGSQAHVTPVAAVPGPAA | |
| | | | | | LPFLYVPGSPTQMNSYGPVVALPTASRSTLAMDT | |
| | | | | | TGLPAPGMLPFCHLWVPVTLVAAGAAQPAASMV | |
| | | | | | MFPHLPALHHHCPHSHRTSQYMPASDGPQAYPD | |
| | | | | | YADQSTNPAFLYKVVKASS | |
| 106 | JHU2 | IPL_ | 0.037500193 | HSFX1 | MEDKRSLSMARCEERNSRGQDHGLERVPFPPQL | Core Human ORF Only |
| | 0559 | RFS | | | QSETYLHPADPSPAWDDPGSTGSPNLRLLTEEIA | |
| | | | | | FQPLAEEASFRRPHPDGDVPPQGEDNLLSLPFP | |
| | | | | | QKLWRLVSSNQFSSIVWVDDSGACRVINQKLFEK | |
| | | | | | EILKRDVAHKVFATTSIKSFFRQLNLYGFRKRRQC | |
| | | | | | TFRTFTRIFSAKRLVSILNKLEFYCHPYFQRDSPH | |
| | | | | | LLVRMKRRVGVKSAPRHQEEDKPEAAGSCLAPAD | |
| | | | | | TEQQDHTSPNENDQVTPQHREPAGPNTQIRSGS | |
| | | | | | APPATPVMVPDSAVASDNSPVTQPAGEWSEGSQ | |
| | | | | | AHVTPVAAVPGPAALPFLYVPGSPTQMNSYGPV | |
| | | | | | VALPTASRSTLAMDTTGLPAPGMLPFCHLWVPVT | |
| | | | | | LVAAGAAQPAASMVMFPHLPALHHHCPHSHRTS | |
| | | | | | QYMPASDGPQAYPDYADQST | |
| 107 | JHU2 | IPL | 0.0946305 | FAN1 | MSPILGYWKIKGLVQPTRLLLEYLEEKYEEHLYER | Full Literal Sequence- |
| | 5078 | RFS | 4 | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMSEGKPPDKKRPR | |
| | | | | | RSLSISKNKKKASNSIISCFNNAPPAKLACPVCSK | |
| | | | | | MVPRYDLNRHLDEMCANNDFVQVDPGQVGLINS | |
| | | | | | NVSMVDLTSVTLEDVTPKKSPPPKTNLTPGQSDS | |
| | | | | | AKREVKQKISPYFKSNDVVCKNQDELRNRSVKVI | |
| | | | | | CLGSLASKLSRKYVKAKKSIDKDEEFAGSSPQSS | |
| | | | | | KSTVVKSLIDNSSEIEDEDQILENSSQKENVFKCD | |
| | | | | | SLKEECIPEHMVRGSKIMEAESQKATRECEKSAL | |
| | | | | | TPGFSDNAIMLFSPDFTLRNTLKSTSEDSLVKQEC | |
| | | | | | IKEVVEKREACHCEEVKMTVASEAKIQLSDSEAKS | |
| | | | | | HSSADDASAWSNIQEAPLQDDSCLNNDIPHSIPLE | |
| | | | | | QGSSCNGPGQTTGHPYYLRSFLVVLKTVLENED | |
| | | | | | DMLLFDEQEKGIVTKFYQLSATGQKLYVRLFQRK | |
| | | | | | LSWIKMTKLEYEEIALDLTPVIEELTNAGFLQTESE | |
| | | | | | LQELSEVLELLSAPELKSLAKTFHLVNPNGQKQQL | |
| | | | | | VDAFLKLAKQRSVCTWGKNKPGIGAVILKRFCWL | |
| | | | | | LLQNPAFLYKVVKASS | |
| 108 | JHU2 | IPL | 0.0946305 | FAN1 | MMSEGKPPDKKRPRRSLSISKNKKKASNSIISCFN | Core Human ORF Only |
| | 5078 | RFS | 4 | | NAPPAKLACPVCSKMVPRYDLNRHLDEMCANND | |
| | | | | | FVQVDPGQVGLINSNVSMVDLTSVTLEDVTPKKS | |
| | | | | | PPPKTNLTPGQSDSAKREVKQKISPYFKSNDVVC | |
| | | | | | KNQDELRNRSVKVICLGSLASKLSRKYVKAKKSID | |
| | | | | | KDEEFAGSSPQSSKSTVVKSLIDNSSEIEDEDQIL | |
| | | | | | ENSSQKENVFKCDSLKEECIPEHMVRGSKIMEAE | |
| | | | | | SQKATRECEKSALTPGFSDNAIMLFSPDFTLRNTL | |
| | | | | | KSTSEDSLVKQECIKEVVEKREACHCEEVKMTVA | |
| | | | | | SEAKIQLSDSEAKSHSSADDASAWSNIQEAPLQD | |
| | | | | | DSCLNNDIPHSIPLEQGSSCNGPGQTTGHPYYLR | |
| | | | | | SFLVVLKTVLENEDDMLLFDEQEKGIVTKFYQLSA | |
| | | | | | TGQKLYVRLFQRKLSWIKMTKLEYEEIALDLTPVIE | |
| | | | | | ELTNAGFLQTESELQELSEVLELLSAPELKSLAKT | |
| | | | | | FHLVNPNGQKQQLVDAFLKLAKQRSVCTWGKNK | |
| | | | | | PGIGAVILKRFCWLLLQ | |
| 109 | JHU2 | IPL | 0.239028734 | PAK1 | MSPILGYWKIKGLVQPTRLLLEYLEEKYEEHLYER | Full Literal Sequence- |
| | 5512 | RFS | | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | |
| | | | | | PKLVCFKKRIEAIPQIDKYLKSSKYIAWPLQGWQA | ORF, occasional ‘post |
| | | | | | TFGGGDHPPKSDLVPRGSPGISGGGGGILGRGS | stop’ end sequence) |
| | | | | | HHHHHHGGGITSLYKKAGTMSNNGLDIQDKPPAP | |
| | | | | | PMRNTSTMIGAGSKDAGTLNHGSKPLPPNPEEK | |
| | | | | | KKKDRFYRSILPGDKTNKKKEKERPEISLPSDFEH | |
| | | | | | TIHVGFDAVTGEFTGMPEQWARLLQTSNITKSEQ | |
| | | | | | KKNPQAVLDVLEFYNSKKTSNSQKYMSFTDKSAE | |
| | | | | | DYNSSNALNVKAVSETPAVPPVSEDEDDDDDDA | |
| | | | | | TPPPVIAPRPEHTKSVYTRSVIEPLPVTPTRDVAT | |
| | | | | | SPISPTENNTTPPDALTRNTEKQKKKPKMSDEEIL | |
| | | | | | EKLRSIVSVGDPKKKYTRFEKIGQGASGTVYTAM | |
| | | | | | DVATGQEVAIKQMNLQQQPKKELIINEILVMRENK | |
| | | | | | NPNIVNYLDSYLVGDELVWVMEYLAGGSLTDVVT | |
| | | | | | ETCMDEGQIAAVCRECLQALEFLHSNQVIHRDIKS | |
| | | | | | DNILLGMDGSVKLTDFGFCAQITPEQSKRSTMVG | |
| | | | | | TPYWMAPEVVTRKAYGPKVDIWSLGIMAIEMIEG | |
| | | | | | EPPYLNENPLRALYLIATNGTPELQNPEKLSAIFR | |
| | | | | | DFLNRCLEMDVEKRGSAKELLQHQFLKIAKPLSSL | |
| | | | | | TPLIAAAKEATKNNHNPAFLYKVVKASS | |
| 110 | JHU2 | IPL | 0.239028734 | PAK1 | MSNNGLDIQDKPPAPPMRNTSTMIGAGSKDAGTL | Core Human ORF Only |
| | 5512 | RFS | | | NHGSKPLPPNPEEKKKKDRFYRSILPGDKTNKKK | |
| | | | | | EKERPEISLPSDFEHTIHVGFDAVTGEFTGMPEQ | |
| | | | | | WARLLQTSNITKSEQKKNPQAVLDVLEFYNSKKT | |
| | | | | | SNSQKYMSFTDKSAEDYNSSNALNVKAVSETPA | |
| | | | | | VPPVSEDEDDDDDDATPPPVIAPRPEHTKSVYTR | |
| | | | | | SVIEPLPVTPTRDVATSPISPTENNTTPPDALTRNT | |
| | | | | | EKQKKKPKMSDEEILEKLRSIVSVGDPKKKYTRFE | |
| | | | | | KIGQGASGTVYTAMDVATGQEVAIKQMNLQQQP | |
| | | | | | KKELIINEILVMRENKNPNIVNYLDSYLVGDELVWV | |
| | | | | | MEYLAGGSLTDVVTETCMDEGQIAAVCRECLQAL | |
| | | | | | EFLHSNQVIHRDIKSDNILLGMDGSVKLTDFGFCA | |
| | | | | | QITPEQSKRSTMVGTPYWMAPEVVTRKAYGPKV | |
| | | | | | DIWSLGIMAIEMIEGEPPYLNENPLRALYLIATNGT | |
| | | | | | PELQNPEKLSAIFRDFLNRCLEMDVEKRGSAKEL | |
| | | | | | LQHQFLKIAKPLSSLTPLIAAAKEATKNNH | |
| 111 | JHU3 | IPL | 0.543800955 | OR2B1 | MSPILGYWKIKGLVQPTRLLLEYLEEKYEEHLYER | Full Literal Sequence- |
| | 0076 | RFS | | 1 | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKSDNHSFLGDSPK | |
| | | | | | AFILLGVSDRPWLELPLFVVLLLSYVLAMLGNVAII | |
| | | | | | LASRVDPQLHSPMYIFLSHLSFLDLCYTTTTVPQM | |
| | | | | | LVNMGSSQKTISYGGCTVQYAVFHWLGCTECIVL | |
| | | | | | AAMALDRYVAICKPLHYAVLMHRALCQQLVALAW | |
| | | | | | LSGFGNSFVQVVLTVQLPFCGRQVLNNFFCEVPA | |
| | | | | | VIKLSCADTAMNDTILAVLVAFFVLVPLALILLSYGF | |
| | | | | | IARAVLRIQSSKGRHKAFGTCSSHLMIVSLFYLPAI | |
| | | | | | YMYLQPPSSYSQEQGKFISLFYSIITPTLNPFTYTL | |
| | | | | | RNKDMKGALRRLLARIWRLCG | |
| 112 | JHU3 | IPL | 0.54380095 | OR2B1 | MKSDNHSFLGDSPKAFILLGVSDRPWLELPLFVV | Core Human ORF Only |
| | 0076 | RFS | 5 | 1 | LLLSYVLAMLGNVAIILASRVDPQLHSPMYIFLSHL | |
| | | | | | SFLDLCYTTTTVPQMLVNMGSSQKTISYGGCTVQ | |
| | | | | | YAVFHWLGCTECIVLAAMALDRYVAICKPLHYAVL | |
| | | | | | MHRALCQQLVALAWLSGFGNSFVQVVLTVQLPF | |
| | | | | | CGRQVLNNFFCEVPAVIKLSCADTAMNDTILAVLV | |
| | | | | | AFFVLVPLALILLSYGFIARAVLRIQSSKGRHKAFG | |
| | | | | | TCSSHLMIVSLFYLPAIYMYLQPPSSYSQEQGKFI | |
| | | | | | SLFYSIITPTLNPFTYTLRNKDMKGALRRLLARIWR | |
| | | | | | LCG | |
| 113 | JHUO | IPL | 0.2721318479 | TMEM1 | MSPILGYWKIKGLVQPTRLLLEYLEEKYEEHLYER | Full Literal Sequence- |
| | 0375 | TO | | 7 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTLFHFGNCFALAYF | |
| | | | | | PYFITYKCSGLSEYNAFWKCVQAGVTYLFVQLCK | |
| | | | | | MLFLATFFPTWEGGIYDFIGEFMKASVDVADLIGL | |
| | | | | | NLVMSRNAGKGEYKIMVAALGWATAELIMSRCIP | |
| | | | | | LWVGARGIEFDWKYIQMSIDSNISLVHYIVASAQV | |
| | | | | | WMITRYDLYHTFRPAVLLLMFLSVYKAFVMETFV | |
| | | | | | HLCSLGSWAALLARAVVTGLLALSTLALYVAVVNV | |
| | | | | | HS | |
| 114 | JHUO | IPI_ | 0.2721318479 | TMEM1 | MTLFHFGNCFALAYFPYFITYKCSGLSEYNAFWK | Core Human ORF Only |
| | 0375 | TO | | 7 | CVQAGVTYLFVQLCKMLFLATFFPTWEGGIYDFIG | |
| | | X | | | EFMKASVDVADLIGLNLVMSRNAGKGEYKIMVAA | |
| | | | | | LGWATAELIMSRCIPLWVGARGIEFDWKYIQMSID | |
| | | | | | SNISLVHYIVASAQVWMITRYDLYHTFRPAVLLLM | |
| | | | | | FLSVYKAFVMETFVHLCSLGSWAALLARAVVTGL | |
| | | | | | LALSTLALYVAVVNVHS | |
| 115 | JHUO | IPI_ | 0.06766526 | PLGRK | MSPILGYWKIKGLVQPTRLLLEYLEEKYEEHLYER | Full Literal Sequence- |
| | 0403 | TO | 5 | T | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGFIFSKSMNESMK | |
| | | | | | NQKEFMLMNARLQLERQLIMQSEMRERQMAMQI | |
| | | | | | AWSREFLKYFGTFFGLAAISLTAGAIKKKKPAFLV | |
| | | | | | PIVPLSFILTYQYDLGYGTLLERMKGEAEDILET | |
| | | | | | EKSKLQLPRGMITFESIEKARKEQSRFFIDK | |
| 116 | JHUO | IPI_ | 0.06766526 | PLGRK | MGFIFSKSMNESMKNQKEFMLMNARLQLERQLI | Core Human ORF Only |
| | 0403 | TO | 5 | T | MQSEMRERQMAMQIAWSREFLKYFGTFFGLAAISL | |
| | | X | | | TAGAIKKKKPAFLVPIVPLSFILTYQYDLGYGTLL | |
| | | | | | ERMKGEAEDILETEKSKLQLPRGMITFESIEKARK | |
| | | | | | EQSRFFIDK | |
| 117 | JHUO | IPI_ | 0.42626019 | SCAMP | MSPILGYWKIKGLVQPTRLLLEYLEEKYEEHLYER | Full Literal Sequence- |
| | 0943 | TO | 3 | 1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSDFDSNPFADPDL | |
| | | | | | NNPFKDPSVTQVTRNVPPGLDEYNPFSDSRTPP | |
| | | | | | PGGVKMPNVPNTQPAIMKPTEEHPAYTQIAKEHA | |
| | | | | | LAQAELLKRQEELERKAAELDRREREMQNLSQH | |
| | | | | | GRKNNWPPLPSNFPVGPCFYQDFSVDIPVEFQK | |
| | | | | | TVKLMYYLWM | |
| 118 | JHUO | IPI_ | 0.42626019 | SCAMP | MSDFDSNPFADPDLNNPFKDPSVTQVTRNVPPG | Core Human ORF Only |
| | 0943 | TO | 3 | 1 | LDEYNPFSDSRTPPPGGVKMPNVPNTQPAIMKPT | |
| | | X | | | EEHPAYTQIAKEHALAQAELLKRQEELERKAAELD | |
| | | | | | RREREMQNLSQHGRKNNWPPLPSNFPVGPCFY | |
| | | | | | QDFSVDIPVEFQKTVKLMYYLWM | |
| 119 | JHUO | IPI_ | 0.38054501 | ASCC1 | MSPILGYWKIKGLVQPTRLLLEYLEEKYEEHLYER | Full Literal Sequence- |
| | 1061 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEVLRPQLIRIDGRN | |
| | | | | | YRKNPVQEQTYQHEEDEEDFYQGSMECADEPC | |
| | | | | | DAYEVEQTPQGFRSTLRAPSLLYKHIVGKRGDTR | |
| | | | | | KKIEMETKTSISIPKPGQDGEIVITGQHRNGVISAR | |
| | | | | | TRIDVLLDTFRRKQPFTHFLAFFLNEVEVQEGFLR | |
| | | | | | FQEEVLAKCSMDHGVDSSIFQNPKKLHLTIGMLVL | |
| | | | | | LSEEEIQQTCEMLQQCKEEFINDISGGKPLEVEMA | |
| | | | | | GIEYMNDDPGMVDVLYAKVHMKDGSNRLQELVD | |
| | | | | | RVLERFQASGLIVKEWNSVKLHATVMNTLFRKDP | |
| | | | | | NAEGRYNLYTAEGKYIFKERESFDGRNILKLFENF | |
| | | | | | YFGSLKLNSIHISQRFTVDSFGNYASCGQIDFS | |
| 120 | JHUO | IPI_ | 0.38054510 | ASCC1 | MEVLRPQLIRIDGRNYRKNPVQEQTYQHEEDEED | Core Human ORF Only |
| | 1061 | TO | 1 | | FYQGSMECADEPCDAYEVEQTPQGFRSTLRAPS | |
| | | X | | | LLYKHIVGKRGDTRKKIEMETKTSISIPKPGQDGEI | |
| | | | | | VITGQHRNGVISARTRIDVLLDTFRRKQPFTHFLA | |
| | | | | | FFLNEVEVQEGFLRFQEEVLAKCSMDHGVDSSIF | |
| | | | | | QNPKKLHLTIGMLVLLSEEEIQQTCEMLQQCKEEF | |
| | | | | | INDISGGKPLEVEMAGIEYMNDDPGMVDVLYAKV | |
| | | | | | HMKDGSNRLQELVDRVLERFQASGLIVKEWNSV | |
| | | | | | KLHATVMNTLFRKDPNAEGRYNLYTAEGKYIFKE | |
| | | | | | RESFDGRNILKLFENFYFGSLKLNSIHISQRFTVDS | |
| | | | | | FGNYASCGQIDFS | |
| 121 | JHUO | IPL | 0.1491688 | C10orf5 | MSPILGYWKIKGLVQPTRLLLEYLEEKYEEHLYER | Full Literal Sequence- |
| | 1065 | TO | 6 | 4 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGVPTALEAGSWR | |
| | | | | | WGSLLFALFLAASLGPVAAFKVATPYSLYVCPEG | |
| | | | | | QNVTLTCRLLGPVDKGHDVTFYKTWYRSSRGEV | |
| | | | | | QTCSERRPIRNLTFQDLHLHHGGHQAANTSHDLA | |
| | | | | | QRHGLESASDHHGNFSITMRNLTLLDSGLYCCLV | |
| | | | | | VEIRHHHSEHRVHGAMELQVQTGKDAPSNCVVY | |
| | | | | | PSSSQESENITAAALATGACIVGILCLPLILLLVY | |
| | | | | | KQRQAASNRRAQELVRMDSNIQGIENPGFEASPPA | |
| | | | | | QGIPEAKVRHPLSYVAQRQPSESGRHLLSEPSTP | |
| | | | | | LSPPGPGDVFFPSLDPVPDSPNFEVI | |
| 122 | JHUO | IPL | 0.1491688 | C10orf5 | MGVPTALEAGSWRWGSLLFALFLAASLGPVAAF | Core Human ORF Only |
| | 1065 | TO | 6 | 4 | KVATPYSLYVCPEGQNVTLTCRLLGPVDKGHDVT | |
| | | X | | | FYKTWYRSSRGEVQTCSERRPIRNLTFQDLHLHH | |
| | | | | | GGHQAANTSHDLAQRHGLESASDHHGNFSITMR | |
| | | | | | NLTLLDSGLYCCLVVEIRHHHSEHRVHGAMELQV | |
| | | | | | QTGKDAPSNCVVYPSSSQESENITAAALATGACI | |
| | | | | | VGILCLPLILLLVYKQRQAASNRRAQELVRMDSNI | |
| | | | | | QGIENPGFEASPPAQGIPEAKVRHPLSYVAQRQP | |
| | | | | | SESGRHLLSEPSTPLSPPGPGDVFFPSLDPVPDS | |
| | | | | | PNFEVI | |
| 123 | JHUO | IPL | 0.13750776 | C21orf3 | MSPILGYWKIKGLVQPTRLLLEYLEEKYEEHLYER | Full Literal Sequence- |
| | 1274 | TO | 4 | 3 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAVRALVASRLAAA | |
| | | | | | SAFTSLSPGGRTPSQRAALHLSVPRPAARVALVL | |
| | | | | | SGCGVYDGTEIHEASAILVHLSRGGAEVQIFAPDV | |
| | | | | | PQMHVIDHTKGQPSEGESRNVLTESARIARGKIT | |
| | | | | | DLANLSAANHDAAIFPGGFGAAKNLSTFAVDGKD | |
| | | | | | CKVNKEVERVLKEFHQAGKPIGLCCIAPVLAAKVL | |
| | | | | | RGVEVTVGHEQEEGGKWPYAGTAEAIKALGAKH | |
| | | | | | CVKEVVEAHVDQKNKVVTTPAFMCETALHYIHDG | |
| | | | | | IGAMVRKVLELTGK | |
| 124 | JHUO | IPL | 0.13750776 | C21orf3 | MAAVRALVASRLAAASAFTSLSPGGRTPSQRAAL | Core Human ORF Only |
| | 1274 | TO | 4 | 3 | HLSVPRPAARVALVLSGCGVYDGTEIHEASAILVH | |
| | | X | | | LSRGGAEVQIFAPDVPQMHVIDHTKGQPSEGESR | |
| | | | | | NVLTESARIARGKITDLANLSAANHDAAIFPGGFG | |
| | | | | | AAKNLSTFAVDGKDCKVNKEVERVLKEFHQAGK | |
| | | | | | PIGLCCIAPVLAAKVLRGVEVTVGHEQEEGGKWP | |
| | | | | | YAGTAEAIKALGAKHCVKEVVEAHVDQKNKVVTT | |
| | | | | | PAFMCETALHYIHDGIGAMVRKVLELTGK | |
| 125 | JHUO | IPL | 0.1953156 | SPATC | MSPILGYWKIKGLVQPTRLLLEYLEEKYEEHLYER | Full Literal Sequence- |
| | 1557 | TO | 5 | 1L | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVRPKKVCFSESSLP | |
| | | | | | TGDRTRRSYYLNEIQSFAGAEKDARVVGEIAFQL | |
| | | | | | DRRILAYVFPGVTRLYGFTVANIPEKIEQTSTKSLD | |
| | | | | | GSVDERKLRELTQRYLALSARLEKLGYSRDVHPA | |
| | | | | | FSEFLINTYGILKQRPDLRANPLHSSPAALRKLVID | |
| | | | | | VVPPKFLGDSLLLLNCLCELSKEDGKPLFAW | |
| 126 | JHUO | IPL | 0.1953156 | SPATC | MVRPKKVCFSESSLPTGDRTRRSYYLNEIQSFAG | Core Human ORF Only |
| | 1557 | TO | 5 | 1L | AEKDARVVGEIAFQLDRRILAYVFPGVTRLYGFTV | |
| | | X | | | ANIPEKIEQTSTKSLDGSVDERKLRELTQRYLALS | |
| | | | | | ARLEKLGYSRDVHPAFSEFLINTYGILKQRPDLRA | |
| | | | | | NPLHSSPAALRKLVIDVVPPKFLGDSLLLLNCLCE | |
| | | | | | LSKEDGKPLFAW | |
| 127 | JHUO | IPL | 0.1135722 | METTL | MSPILGYWKIKGLVQPTRLLLEYLEEKYEEHLYER | Full Literal Sequence- |
| | 2352 | TO | 2 | 9 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTSGPGGPAAAAGG | |
| | | | | | RKENHQWYVCNREKLCESLQAVFVQSYLDQGTQ | |
| | | | | | IFLNNSIEKSGWLFIQLYHSFVSSVFSLFMSRTSIN | |
| | | | | | GLLGRGSMFVFSPDQFQRLLKINPDWKTHRLLDL | |
| | | | | | GAGDGEVTKIMSPHFEEIYATELSETMIWQLQKK | |
| | | | | | KYRVLGINEWQNTGFQYDVISCLNLLDRCDQPLT | |
| | | | | | LLKDIRSVLEPTRGRVILALVLPFHPYVENVGGKW | |
| | | | | | EKPSEILEIKGQNWEEQVNSLPEVFRKAGFVIEAF | |
| | | | | | TRLPYLCEGDMYNDYYVLDDAVFVLKPV | |
| 128 | JHUO | IPI_ | 0.1135722 | METTL | MTSGPGGPAAAAGGRKENHQWYVCNREKLCES | Core Human ORF Only |
| | 2352 | TO | 2 | 9 | LQAVFVQSYLDQGTQIFLNNSIEKSGWLFIQLYHS | |
| | | X | | | FVSSVFSLFMSRTSINGLLGRGSMFVFSPDQFQR | |
| | | | | | LLKINPDWKTHRLLDLGAGDGEVTKIMSPHFEEIY | |
| | | | | | ATELSETMIWQLQKKKYRVLGINEWQNTGFQYDV | |
| | | | | | ISCLNLLDRCDQPLTLLKDIRSVLEPTRGRVILALV | |
| | | | | | LPFHPYVENVGGKWEKPSEILEIKGQNWEEQVNS | |
| | | | | | LPEVFRKAGFVIEAFTRLPYLCEGDMYNDYYVLD | |
| | | | | | DAVFVLKPV | |
| 129 | JHUO | IPI_ | 0.17028771 | CXCL1 | MSPILGYWKIKGLVQPTRLLLEYLEEKYEEHLYER | Full Literal Sequence- |
| | 3287 | TO | 9 | 6 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSGSQSEVAPSPQS | |
| | | | | | PRSPEMGRDLRPGSRVLLLLLLLLLVYLTQPGNG | |
| | | | | | NEGSVTGSCYCGKRISSDSPPSVQFMNRLRKHL | |
| | | | | | RAYHRCLYYTRFQLLSWSVCGGNKDPVWQELM | |
| | | | | | SCLDLKECGHAYSGIVAHQKHLLPTSPPISQASEG | |
| | | | | | ASSDIHTPAQMLLSTLQSTQRPTLPVGSLSSDKEL | |
| | | | | | TRPNETTIHTAGHSLAAGPEAGENQKQPEKNAGP | |
| | | | | | TARTSATVPVLCLLAIIFILTAALSYVLCKRRRGQS | |
| | | | | | PQSSPDLPVHYIPVAPDSNT | |
| 130 | JHUO | IPI_ | 0.17028771 | CXCL1 | MSGSQSEVAPSPQSPRSPEMGRDLRPGSRVLLL | Core Human ORF Only |
| | 3287 | TO | 9 | 6 | LLLLLLVYLTQPGNGNEGSVTGSCYCGKRISSDS | |
| | | X | | | PPSVQFMNRLRKHLRAYHRCLYYTRFQLLSWSV | |
| | | | | | CGGNKDPVWQELMSCLDLKECGHAYSGIVAHQK | |
| | | | | | HLLPTSPPISQASEGASSDIHTPAQMLLSTLQSTQ | |
| | | | | | RPTLPVGSLSSDKELTRPNETTIHTAGHSLAAGPE | |
| | | | | | AGENQKQPEKNAGPTARTSATVPVLCLLAIIFILTA | |
| | | | | | ALSYVLCKRRRGQSPQSSPDLPVHYIPVAPDSNT | |
| 131 | JHUO | IPI_ | 0.60805384 | SPX | MSPILGYWKIKGLVQPTRLLLEYLEEKYEEHLYER | Full Literal Sequence- |
| | 3472 | TO | 2 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKGLRSLAATTLALF | |
| | | | | | LVFVFLGNSSCAPQRLLERRNWTPQAMLYLKGA | |
| | | | | | QGRRFISDQSRRKDLSDRPLPERRSPNPQLLTIP | |
| | | | | | EAATILLASLQKSPEDEEKNFDQTRFLEDSLLNW | |
| 132 | JHUO | IPI_ | 0.608053842 | SPX | MKGLRSLAATTLALFLVFVFLGNSSCAPQRLLER | Core Human ORF Only |
| | 3472 | TO | | | RNWTPQAMLYLKGAQGRRFISDQSRRKDLSDRP | |
| | | X | | | LPERRSPNPQLLTIPEAATILLASLQKSPEDEEKNF | |
| | | | | | DQTRFLEDSLLNW | |
| 133 | JHUO | IPL | 0.07100168 | TRIM48 | MSPILGYWKIKGLVQPTRLLLEYLEEKYEEHLYER | Full Literal Sequence- |
| | 3541 | TO | 6 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNSGISQVFQRELTC | |
| | | | | | PICMNYFIDPVTIDCGHSFCRPCFYLNWQDIPILTQ | |
| | | | | | CFECIKTIQQRNLKTNIRLKKMASLARKASLWLFL | |
| | | | | | SSEEQMCGIHRETKKMFCEVDRSLLCLLCSSSQE | |
| | | | | | HRYHRHCPAEWAAEEHWEKLLKKMQSLWEKAC | |
| | | | | | ENQRNLNVETTRISHWKAFGDILYRSESVLLHMP | |
| | | | | | QPLNLALRAGPITGLRDRLNQF | |
| 134 | JHUO | IPI_ | 0.07100168 | TRIM48 | MNSGISQVFQRELTCPICMNYFIDPVTIDCGHSFC | Core Human ORF Only |
| | 3541 | TO | 6 | | RPCFYLNWQDIPILTQCFECIKTIQQRNLKTNIRLK | |
| | | X | | | KMASLARKASLWLFLSSEEQMCGIHRETKKMFCE | |
| | | | | | VDRSLLCLLCSSSQEHRYHRHCPAEWAAEEHWE | |
| | | | | | KLLKKMQSLWEKACENQRNLNVETTRISHWKAF | |
| | | | | | GDILYRSESVLLHMPQPLNLALRAGPITGLRDRLN | |
| | | | | | QF | |
| 135 | JHUO | IPL | 0.5223457 | CFAP6 | MSPILGYWKIKGLVQPTRLLLEYLEEKYEEHLYER | Full Literal Sequence- |
| | 3661 | TO | 2 | 1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSYALSHTNRKLTLE | |
| | | | | | PKITVNAKIIVVGASSVGISFLETLVFCSHMKFNNL | |
| | | | | | TLISTHGLPGKKLLDTEQRKFLASDHCFNDKDYAL | |
| | | | | | MSLCSWVNVVVGRMTGIDRAAKHVVLSTDEIVPY | |
| | | | | | DHLILCTGQQYQVPCPTEADISQHLTNREVPNSS | |
| | | | | | QRRYTGKVPCNHFTLNEEEDCFKALIWIRNNSITT | |
| | | | | | EDGGRASVLFC | |
| 136 | JHUO | IPL | 0.5223457 | CFAP6 | MSYALSHTNRKLTLEPKITVNAKIIVVGASSVGISF | Core Human ORF Only |
| | 3661 | TO | 2 | 1 | LETLVFCSHMKFNNLTLISTHGLPGKKLLDTEQRK | |
| | | X | | | FLASDHCFNDKDYALMSLCSWVNVVVGRMTGID | |
| | | | | | RAAKHVVLSTDEIVPYDHLILCTGQQYQVPCPTEA | |
| | | | | | DISQHLTNREVPNSSQRRYTGKVPCNHFTLNEEE | |
| | | | | | DCFKALIWIRNNSITTEDGGRASVLFC | |
| 137 | JHUO | IPL | 0.2533092 | IQCB1 | MSPILGYWKIKGLVQPTRLLLEYLEEKYEEHLYER | Full Literal Sequence- |
| | 4082 | TO | 7 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKPTGTDPRILSIAAEVA | |
| | | | | | KSPEQNVPVILLKLKEIINITPLGSSELKKIKQDIY | |
| | | | | | CYDLIQYCLLVLSQDYSRIQGGWTTISQLTQILSH | |
| | | | | | CCVGLEPGEDAEEFYNELLPSAAENFLVLGRQLQ | |
| | | | | | TCFINAAKAEEKDELLHFFQIVTDSLFWLLGGHVE | |
| | | | | | LIQNVLQSDHFLHLLQADNVQIGSAVMMMLQNIL | |
| | | | | | QINRSKRSKMLLEINRQKEEEDLKLQLQLQRQRA | |
| | | | | | MRLSRELQLSMLEIVHPGQVEKHYREMEEKSALII | |
| | | | | | QKHWRGYRERKNFHQQRQSLIEYKAAVTLQRAA | |
| | | | | | LKFLAKYRKKKKLFAPWRGLQELTDARRVELKKR | |
| | | | | | VDDYVRRHLGSPMSDVVSRELHAQAQERLQHYF | |
| | | | | | MGRALEERAQQHREALIAQISTNVEQLMKAPSLK | |
| | | | | | EAEGKEPELFLSRSRPVAAKAKQAHLTTLKHIQAP | |
| | | | | | WWKKLGEESGDEIDVPKDELSIELENLFIGGTKPP | |
| 138 | JHUO | IPL | 0.2533092 | IQCB1 | MKPTGTDPRILSIAAEVAKSPEQNVPVILLKLKEIIN | Core Human ORF Only |
| | 4082 | TO | 7 | | ITPLGSSELKKIKQDIYCYDLIQYCLLVLSQDYSRI | |
| | | X | | | QGGWTTISQLTQILSHCCVGLEPGEDAEEFYNELL | |
| | | | | | PSAAENFLVLGRQLQTCFINAAKAEEKDELLHFFQ | |
| | | | | | IVTDSLFWLLGGHVELIQNVLQSDHFLHLLQADNV | |
| | | | | | QIGSAVMMMLQNILQINRSKRSKMLLEINRQKEEE | |
| | | | | | DLKLQLQLQRQRAMRLSRELQLSMLEIVHPGQVE | |
| | | | | | KHYREMEEKSALIIQKHWRGYRERKNFHQQRQS | |
| | | | | | LIEYKAAVTLQRAALKFLAKYRKKKKLFAPWRGLQ | |
| | | | | | ELTDARRVELKKRVDDYVRRHLGSPMSDVVSRE | |
| | | | | | LHAQAQERLQHYFMGRALEERAQQHREALIAQIS | |
| | | | | | TNVEQLMKAPSLKEAEGKEPELFLSRSRPVAAKA | |
| | | | | | KQAHLTTLKHIQAPVWVKKLGEESGDEIDVPKDEL | |
| | | | | | SIELENLFIGGTKPP | |
| 139 | JHUO | IPL | 0.21517061 | IGHA1 | MSPILGYWKIKGLVQPTRLLLEYLEEKYEEHLYER | Full Literal Sequence- |
| | 4166 | TO | 8 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEFGLSVWFLVAILK | |
| | | | | | GVQCEVQLVESGGGVVRPGGSLRLSCATSGFTF | |
| | | | | | DDSGASWVRQAPGKGLEWVSSINWNGGSTNYA | |
| | | | | | DSVKGRFTISRDNAKNSLYLQMNSLRVEDTALYY | |
| | | | | | CARDPTKYCSGGSCLGYYMDVWGKGTTVTVSS | |
| | | | | | ASPTSPKVFPLSLCSTQPDGNVVIACLVQGFFPQ | |
| | | | | | EPLSVTWSESGQGVTARNFPPSQDASGDLYTTS | |
| | | | | | SQLTLPATQCLAGKSVTCHVKHYTNPSQDVTVPC | |
| | | | | | PVPSTPPTPSPSTPPTPSPSCCHPRLSLHRPALE | |
| | | | | | DLLLGSEANLTCTLTGLRDASGVTFTWTPSSGKS | |
| | | | | | AVQGPPDRDLCGCYSVSSVLPGCAEPWNHGKTF | |
| | | | | | TCTAAYPESKTPLTATLSKSGNTFRPEVHLLPPPS | |
| | | | | | EELALNELVTLTCLARGFSPKDVLVRWLQGSQEL | |
| | | | | | PREKYLTWASRQEPSQGTTTFAVTSILRVAAEDW | |
| | | | | | KKGDTFSCMVGHEALPLAFTQKTIDRLAGKPTHV | |
| | | | | | NVSVVMAEVDGTCY | |
| 140 | JHUO | IPL | 0.21517061 | IGHA1 | MEFGLSWVFLVAILKGVQCEVQLVESGGGVVRP | Core Human ORF Only |
| | 4166 | TO | 8 | | GGSLRLSCATSGFTFDDSGASWVRQAPGKGLE | |
| | | X | | | WVSSINWNGGSTNYADSVKGRFTISRDNAKNSL | |
| | | | | | YLQMNSLRVEDTALYYCARDPTKYCSGGSCLGY | |
| | | | | | YMDVWGKGTTVTVSSASPTSPKVFPLSLCSTQP | |
| | | | | | DGNVVIACLVQGFFPQEPLSVTWSESGQGVTAR | |
| | | | | | NFPPSQDASGDLYTTSSQLTLPATQCLAGKSVTC | |
| | | | | | HVKHYTNPSQDVTVPCPVPSTPPTPSPSTPPTPS | |
| | | | | | PSCCHPRLSLHRPALEDLLLGSEANLTCTLTGLR | |
| | | | | | DASGVTFTWTPSSGKSAVQGPPDRDLCGCYSVS | |
| | | | | | SVLPGCAEPWNHGKTFTCTAAYPESKTPLTATLS | |
| | | | | | KSGNTFRPEVHLLPPPSEELALNELVTLTCLARGF | |
| | | | | | SPKDVLVRWLQGSQELPREKYLTWASRQEPSQG | |
| | | | | | TTTFAVTSILRVAAEDWKKGDTFSCMVGHEALPL | |
| | | | | | AFTQKTIDRLAGKPTHVNVSVVMAEVDGTCY | |
| 141 | JHUO | IPL | 0.36334213 | ATF5 | MSPILGYWKIKGLVQPTRLLLEYLEEKYEEHLYER | Full Literal Sequence- |
| | 4233 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSLLATLGLELDRAL | |
| | | | | | LPASGLGWLVDYGKLPPAPAPLAPYEVLGGALEG | |
| | | | | | GLPVGGEPLAGDGFSDWMTERVDFTALLPLEPP | |
| | | | | | LPPGTLPQPSPTPPDLEAMASLLKKELEQMEDFF | |
| | | | | | LDAPLLPPPSPPPLPPPPLPPAPSLPLSLPSFDLP | |
| | | | | | QPPVLDTLDLLAIYCRNEAGQEEVGMPPLPPPQQ | |
| | | | | | PPPPSPPQPSRLAPYPHPATTRGDRKQKKRDQN | |
| | | | | | KSAALRYRQRKRAEGEALEGECQGLEARNRELK | |
| | | | | | ERAESVEREIQYVKDLLIEVYKARSQRTRSC | |
| 142 | JHUO | IPL | 0.36334213 | ATF5 | MSLLATLGLELDRALLPASGLGWLVDYGKLPPAP | Core Human ORF Only |
| | 4233 | TO | 1 | | APLAPYEVLGGALEGGLPVGGEPLAGDGFSDWM | |
| | | X | | | TERVDFTALLPLEPPLPPGTLPQPSPTPPDLEAMA | |
| | | | | | SLLKKELEQMEDFFLDAPLLPPPSPPPLPPPPLPP | |
| | | | | | APSLPLSLPSFDLPQPPVLDTLDLLAIYCRNEAGQ | |
| | | | | | EEVGMPPLPPPQQPPPPSPPQPSRLAPYPHPAT | |
| | | | | | TRGDRKQKKRDQNKSAALRYRQRKRAEGEALEG | |
| | | | | | ECQGLEARNRELKERAESVEREIQYVKDLLIEVYK | |
| | | | | | ARSQRTRSC | |
| 143 | JHUO | IPL | 0.22334166 | ALDH3 | MSPILGYWKIKGLVQPTRLLLEYLEEKYEEHLYER | Full Literal Sequence- |
| | 4610 | TO | 4 | A1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSKISEAVKRARAAF | |
| | | | | | SSGRTRPLQFRIQQLEALQRLIQEQEQELVGALA | |
| | | | | | ADLHKNEWNAYYEEVVYVLEEIEYMIQKLPEWAA | |
| | | | | | DEPVEKTPQTQQDELYIHSEPLGVVLVIGTWNYP | |
| | | | | | FNLTIQPMVGAIAAGNAVVLKPSELSENMASLLATI | |
| | | | | | IPQYLDKDLYPVINGGVPETTELLKERFDHILYTGS | |
| | | | | | TGVGKIIMTAAAKHLTPVTLELGGKSPCYVDKNCD | |
| | | | | | LDVACRRIAWGKFMNSGQTCVAPDYILCDPSIQN | |
| | | | | | QIVEKLKKSLKEFYGEDAKKSRDYGRIISARHFQR | |
| | | | | | VMGLIEGQKVAYGGTGDAATRYIAPTILTDVDPQS | |
| | | | | | PVMQEEIFGPVLPIVCVRSLEEAIQFINQREKPLAL | |
| | | | | | YMFSSNDKVIKKMIAETSSGGVAANDVIVHITLHSL | |
| | | | | | PFGGVGNSGMGSYHGKKSFETFSHRRSCLVRPL | |
| | | | | | MNDEGLKVRYPPSPAKMTQH | |
| 144 | JHUO | IPL | 0.22334166 | ALDH3 | MSKISEAVKRARAAFSSGRTRPLQFRIQQLEALQ | Core Human ORF Only |
| | 4610 | TO | 4 | A1 | RLIQEQEQELVGALAADLHKNEWNAYYEEVVYVL | |
| | | X | | | EEIEYMIQKLPEWAADEPVEKTPQTQQDELYIHSE | |
| | | | | | PLGVVLVIGTWNYPFNLTIQPMVGAIAAGNAVVLK | |
| | | | | | PSELSENMASLLATIIPQYLDKDLYPVINGGVPETT | |
| | | | | | ELLKERFDHILYTGSTGVGKIIMTAAAKHLTPVTLE | |
| | | | | | LGGKSPCYVDKNCDLDVACRRIAWGKFMNSGQT | |
| | | | | | CVAPDYILCDPSIQNQIVEKLKKSLKEFYGEDAKK | |
| | | | | | SRDYGRIISARHFQRVMGLIEGQKVAYGGTGDAA | |
| | | | | | TRYIAPTILTDVDPQSPVMQEEIFGPVLPIVCVRSL | |
| | | | | | EEAIQFINQREKPLALYMFSSNDKVIKKMIAETSSG | |
| | | | | | GVAANDVIVHITLHSLPFGGVGNSGMGSYHGKKS | |
| | | | | | FETFSHRRSCLVRPLMNDEGLKVRYPPSPAKMT | |
| | | | | | QH | |
| 145 | JHUO | IPL | 0.29676565 | SPON2 | MSPILGYWKIKGLVQPTRLLLEYLEEKYEEHLYER | Full Literal Sequence- |
| | 5162 | TO | 5 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMENPSPAAALGKAL | |
| | | | | | CALLLATLGAAGQPLGGESICSARAPAKYSITFTG | |
| | | | | | KWSQTAFPKQYPLFRPPAQWSSLLGAAHSSDYS | |
| | | | | | MWRKNQYVSNGLRDFAERGEAWALMKEIEAAG | |
| | | | | | EALQSVHEVFSAPAVPSGTGQTSAELEVQRRHSL | |
| | | | | | VSFVVRIVPSPDWFVGVDSLDLCDGDRWREQAA | |
| | | | | | LDLYPYDAGTDSGFTFSSPNFATIPQDTVTEITSS | |
| | | | | | SPSHPANSFYYPRLKALPPIARVTLLRLRQSPRAF | |
| | | | | | IPPAPVLPSRDNEIVDSASVPETPLDCEVSLWSS | |
| | | | | | WGLCGGHCGRLGTKSRTRYVRVQPANNGSPCP | |
| | | | | | ELEEEAECVPDNCV | |
| 146 | JHUO | IPL | 0.29676565 | SPON2 | MENPSPAAALGKALCALLLATLGAAGQPLGGESI | Core Human ORF Only |
| | 5162 | TO | 5 | | CSARAPAKYSITFTGKWSQTAFPKQYPLFRPPAQ | |
| | | X | | | WSSLLGAAHSSDYSMWRKNQYVSNGLRDFAER | |
| | | | | | GEAWALMKEIEAAGEALQSVHEVFSAPAVPSGT | |
| | | | | | GQTSAELEVQRRHSLVSFVVRIVPSPDWFVGVDS | |
| | | | | | LDLCDGDRWREQAALDLYPYDAGTDSGFTFSSP | |
| | | | | | NFATIPQDTVTEITSSSPSHPANSFYYPRLKALPPI | |
| | | | | | ARVTLLRLRQSPRAFIPPAPVLPSRDNEIVDSASV | |
| | | | | | PETPLDCEVSLWSSWGLCGGHCGRLGTKSRTRY | |
| | | | | | VRVQPANNGSPCPELEEEAECVPDNCV | |
| 147 | JHUO | IPL | 0.1007290 | PLAUR | MSPILGYWKIKGLVQPTRLLLEYLEEKYEEHLYER | Full Literal Sequence- |
| | 5438 | TO | 4 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGHPPLLPLLLLLHT | |
| | | | | | CVPASWGLRCMQCKTNGDCRVEECALGQDLCR | |
| | | | | | TTIVRLWEEGEELELVEKSCTHSEKTNRTLSYRT | |
| | | | | | GLKITSLTEVVCGLDLCNQGNSGRAVTYSRSRYL | |
| | | | | | ECISCGSSDMSCERGRHQSLQCRSPEEQCLDVV | |
| | | | | | THWIQEGEEGRPKDDRHLRGCGYLPGCPGSNG | |
| | | | | | FHNNDTFHFLKCCNTTKCNEGPILELENLPQNGR | |
| | | | | | QCYSCKGNSTHGCSSEETFLIDCRGPMNQCLVA | |
| | | | | | TGTHEPKNQSYMVRGCATASMCQHAHLGDAFS | |
| | | | | | MNHIDVSCCTKSGCNHPDLDVQYRSGAAPQPGP | |
| | | | | | AHLSLTITLLMTARLWGGTLLWT | |
| 148 | JHUO | IPL | 0.1007290 | PLAUR | MGHPPLLPLLLLLHTCVPASWGLRCMQCKTNGD | Core Human ORF Only |
| | 5438 | TO | 4 | | CRVEECALGQDLCRTTIVRLWEEGEELELVEKSC | |
| | | X | | | THSEKTNRTLSYRTGLKITSLTEVVCGLDLCNQG | |
| | | | | | NSGRAVTYSRSRYLECISCGSSDMSCERGRHQS | |
| | | | | | LQCRSPEEQCLDVVTHWIQEGEEGRPKDDRHLR | |
| | | | | | GCGYLPGCPGSNGFHNNDTFHFLKCCNTTKCNE | |
| | | | | | GPILELENLPQNGRQCYSCKGNSTHGCSSEETFL | |
| | | | | | IDCRGPMNQCLVATGTHEPKNQSYMVRGCATAS | |
| | | | | | MCQHAHLGDAFSMNHIDVSCCTKSGCNHPDLDV | |
| | | | | | QYRSGAAPQPGPAHLSLTITLLMTARLWGGTLLW | |
| | | | | | T | |
| 149 | JHUO | IPL | 0.14173545 | CALHM | MSPILGYWKIKGLVQPTRLLLEYLEEKYEEHLYER | Full Literal Sequence- |
| | 5595 | TO | 1 | 1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMDKFRMIFQFLQS | |
| | | | | | NQESFMNGICGIMALASAQMYSAFDFNCPCLPGY | |
| | | | | | NAAYSAGILLAPPLVLFLLGLVMNNNVSMLAEEW | |
| | | | | | KRPPGRRAKDPAVLRYMFCSMAQRALIAPVVVWV | |
| | | | | | AVTLLDGKCFLCAFCTAVPVSALGNGSLAPGLPA | |
| | | | | | PELARLLARVPCPEIYDGDWLLAREVAVRYLRCIS | |
| | | | | | QALGWSFVLLTTLLAFVVRSVRPCFTQAAFLKSK | |
| | | | | | YWSHYIDIERKLFDETCTEHAKAFAKVCIQQFFEA | |
| | | | | | MNHDLELGHTNGTLATAPASAAAPTTPDGAEEER | |
| | | | | | EKLRGITDQGTMNRLLTSWHKCKPPLRLGQEEPP | |
| | | | | | LMGNGWAGGGPRPPRKEVATYFSKV | |
| 150 | JHUO | IPI_ | 0.14173545 | CALHM | MMDKFRMIFQFLQSNQESFMNGICGIMALASAQ | Core Human ORF Only |
| | 5595 | TO | 1 | 1 | MYSAFDFNCPCLPGYNAAYSAGILLAPPLVLFLLG | |
| | | X | | | LVMNNNVSMLAEEWKRPPGRRAKDPAVLRYMF | |
| | | | | | CSMAQRALIAPVVVWVAVTLLDGKCFLCAFCTAVP | |
| | | | | | VSALGNGSLAPGLPAPELARLLARVPCPEIYDGD | |
| | | | | | WLLAREVAVRYLRCISQALGWSFVLLTTLLAFVVR | |
| | | | | | SVRPCFTQAAFLKSKYWSHYIDIERKLFDETCTEH | |
| | | | | | AKAFAKVCIQQFFEAMNHDLELGHTNGTLATAPA | |
| | | | | | SAAAPTTPDGAEEEREKLRGITDQGTMNRLLTSW | |
| | | | | | HKCKPPLRLGQEEPPLMGNGWAGGGPRPPRKE | |
| | | | | | VATYFSKV | |
| 151 | JHUO | IPI_ | 0.37145002 | NUP62 | MSPILGYWKIKGLVQPTRLLLEYLEEKYEEHLYER | Full Literal Sequence- |
| | 5623 | TO | 5 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSGFNFGGTGAPTG | |
| | | | | | GFTFGTAKTATTTPATGFSFSTSGTGGFNFGAPF | |
| | | | | | QPATSTPSTGLFSLATQTPATQTTGFTFGTATLAS | |
| | | | | | GGTGFSLGIGASKLNLSNTAATPAMANPSGFGLG | |
| | | | | | SSNLTNAISSTVTSSQGTAPTGFVFGPSTTSVAPA | |
| | | | | | TTSGGFSFTGGSTAQPSGFNIGSAGNSAQPTAPA | |
| | | | | | TLPFTPATPAATTAGATQPAAPTPTATITSTGPSL | |
| | | | | | FASIATAPTSSATTGLSLCTPVTTAGAPTAGTQGF | |
| | | | | | SLKAPGAASGTSTTTSTAATATATTTSSSSTTGFA | |
| | | | | | LNLKPLAPAGIPSNTAAAVTAPPGPGAAAGAAAS | |
| | | | | | SAMTYAQLESLINKWSLELEDQERHFLQQATQVN | |
| | | | | | AWDRTLIENGEKITSLHREVEKVKLDQKRLDQEL | |
| | | | | | DFILSQQKELEDLLSPLEELVKEQSGTIYLQHADE | |
| | | | | | EREKTYKLAENIDAQLKRMAQDLKDIIEHLNTSGA | |
| | | | | | PADTSDPLQQICKILNAHMDSLQWIDQNSALLQR | |
| | | | | | KVEEVTKVCEGRRKEQERSFRITFD | |
| 152 | JHUO | IPI_ | 0.37145002 | NUP62 | MSGFNFGGTGAPTGGFTFGTAKTATTTPATGFSF | Core Human ORF Only |
| | 5623 | TO | 5 | | STSGTGGFNFGAPFQPATSTPSTGLFSLATQTPA | |
| | | X | | | TQTTGFTFGTATLASGGTGFSLGIGASKLNLSNTA | |
| | | | | | ATPAMANPSGFGLGSSNLTNAISSTVTSSQGTAP | |
| | | | | | TGFVFGPSTTSVAPATTSGGFSFTGGSTAQPSGF | |
| | | | | | NIGSAGNSAQPTAPATLPFTPATPAATTAGATQP | |
| | | | | | AAPTPTATITSTGPSLFASIATAPTSSATTGLSLCT | |
| | | | | | PVTTAGAPTAGTQGFSLKAPGAASGTSTTTSTAA | |
| | | | | | TATATTTSSSSTTGFALNLKPLAPAGIPSNTAAAV | |
| | | | | | TAPPGPGAAAGAAASSAMTYAQLESLINKWSLEL | |
| | | | | | EDQERHFLQQATQVNAWDRTLIENGEKITSLHRE | |
| | | | | | VEKVKLDQKRLDQELDFILSQQKELEDLLSPLEEL | |
| | | | | | VKEQSGTIYLQHADEEREKTYKLAENIDAQLKRM | |
| | | | | | AQDLKDIIEHLNTSGAPADTSDPLQQICKILNAHM | |
| | | | | | DSLQWIDQNSALLQRKVEEVTKVCEGRRKEQER | |
| | | | | | SFRITFD | |
| 153 | JHUO | IPI_ | 0.6157559 | KCNA2 | MSPILGYWKIKGLVQPTRLLLEYLEEKYEEHLYER | Full Literal Sequence- |
| | 5901 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTVATGDPADEAAA | |
| | | | | | LPGHPQDTYDPEADHECCERVVINISGLRFETQL | |
| | | | | | KTLAQFPETLLGDPKKRMRYFDPLRNEYFFDRNR | |
| | | | | | PSFDAILYYYQSGGRLRRPVNVPLDIFSEEIRFYEL | |
| | | | | | GEEAMEMFREDEGYIKEEERPLPENEFQRQVWL | |
| | | | | | LFEYPESSGPARIIAIVSVMVILISIVSFCLETLPI | |
| | | | | | DFRENEDMHGSGVTFHTYSNSTIGYQQSTSFTDPFF | |
| | | | | | IVETLCIIWFSFEFLVRFFACPSKAGFFTNIMNIID | |
| | | | | | IVAIIPYFITLGTELAEKPEDAQQGQQAMSLAILRV | |
| | | | | | IRLERRPLQSQKSKRGRQHLNTSHDCTLGINLVAG | |
| | | | | | MTVQWTRASGPDDRQTPAVTTLHRMY | |
| 154 | JHUO | IPI_ | 0.6157559 | KCNA2 | MTVATGDPADEAAALPGHPQDTYDPEADHECCE | Core Human ORF Only |
| | 5901 | TO | 1 | | RVVINISGLRFETQLKTLAQFPETLLGDPKKRMRY | |
| | | X | | | FDPLRNEYFFDRNRPSFDAILYYYQSGGRLRRPV | |
| | | | | | NVPLDIFSEEIRFYELGEEAMEMFREDEGYIKEEE | |
| | | | | | RPLPENEFQRQVWLLFEYPESSGPARIIAIVSVMV | |
| | | | | | ILISIVSFCLETLPIFRDENEDMHGSGVTFHTYSNS | |
| | | | | | TIGYQQSTSFTDPFFIVETLCIIWFSFEFLVRFFAC | |
| | | | | | PSKAGFFTNIMNIIDIVAIIPYFITLGTELAEKPED | |
| | | | | | AQQGQQAMSLAILRVIRLERRPLQSQKSKRGRQHLN | |
| | | | | | TSHDCTLGINLVAGMTVQWTRASGPDDRQTPAV | |
| | | | | | TTLHRMY | |
| 155 | JHUO | IPL | 0.25184553 | SLC23 | MSPILGYWKIKGLVQPTRLLLEYLEEKYEEHLYER | Full Literal Sequence- |
| | 6217 | TO | 1 | A1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMRAQEDLEGRTQHE | |
| | | | | | TTRDPSTPLPTEPKFDMLYKIEDVPPWYLCILLGF | |
| | | | | | QHYLTCFSGTIAVPFLLAEALCVGHDQHMVSQLIGTI | |
| | | | | | FTCVGITTLIQTTVGIRLPLFQASAFAFLVPAKAIL | |
| | | | | | ALERWKCPPEEEIYGNWSLPLNTSHIWHPRIREV | |
| | | | | | GLHVQGAIMVSSVVEVVIGLLGLPGALLNYIGPLT | |
| | | | | | VTPTVSLIGLSVFQAAGDRAGSHWGISACSILLIIL | |
| | | | | | FSQYLRNLTFLLPVYRWGKGLTLLRIQIFKMFPIML | |
| | | | | | AIMTVWLLCYVLTLTDVLPTDPKAYGFQARTDAR | |
| | | | | | GDIMAIAPWIRIPYPCQWGLPTVTAAAVLGMFSAT | |
| | | | | | LAGIIESIGDYYACARLAGAPPPPVHAINRGIFTEGI | |
| | | | | | CCIIAGLLGTGNGSTSSSPNIGVLGITKVGSRRVV | |
| | | | | | QYGAAIMLVLGTIGKFTALFASLPDPILGGMFCTLF | |
| | | | | | GMITAVGLSNLQFVDMNSSRNLFVLGFSMFFGLT | |
| | | | | | LPNYLESNPGAINTGILEVDQILIVLLTTEMFVGGC | |
| | | | | | LAFILDNTVPGSPEERGLIQWKAGAHANSDMSSS | |
| | | | | | LKSYDFPIGMGIVKRITFLKYIPICPVFKGFSSSSKD | |
| | | | | | QIAIPEDTPENTETASVCTKV | |
| 156 | JHUO | IPL | 0.25184553 | SLC23 | MRAQEDLEGRTQHETTRDPSTPLPTEPKFDMLY | Core Human ORF Only |
| | 6217 | TO | 1 | A1 | KIEDVPPWYLCILLGFQHYLTCFSGTIAVPFLLAEA | |
| | | X | | | LCVGHDQHMVSQLIGTIFTCVGITTLIQTTVGIRLP | |
| | | | | | LFQASAFAFLVPAKAILALERWKCPPEEEIYGNWS | |
| | | | | | LPLNTSHIWHPRIREVGLHVQGAIMVSSVVEVVIG | |
| | | | | | LLGLPGALLNYIGPLTVTPTVSLIGLSVFQAAGDRA | |
| | | | | | GSHWGISACSILLIILFSQYLRNLTFLLPVYRWGKG | |
| | | | | | LTLLRIQIFKMFPIMLAIMTVWLLCYVLTLTDVLPTD | |
| | | | | | PKAYGFQARTDARGDIMAIAPWIRIPYPCQWGLP | |
| | | | | | TVTAAAVLGMFSATLAGIIESIGDYYACARLAGAP | |
| | | | | | PPPVHAINRGIFTEGICCIIAGLLGTGNGSTSSSPNI | |
| | | | | | GVLGITKVGSRRVVQYGAAIMLVLGTIGKFTALFA | |
| | | | | | SLPDPILGGMFCTLFGMITAVGLSNLQFVDMNSS | |
| | | | | | RNLFVLGFSMFFGLTLPNYLESNPGAINTGILEVD | |
| | | | | | QILIVLLTTEMFVGGCLAFILDNTVPGSPEERGLIQ | |
| | | | | | WKAGAHANSDMSSSLKSYDFPIGMGIVKRITFLK | |
| | | | | | YIPICPVFKGFSSSSKDQIAIPEDTPENTETASVCT | |
| | | | | | KV | |
| 157 | JHUO | IPL | 0.31554283 | SLC34 | MSPILGYWKIKGLVQPTRLLLEYLEEKYEEHLYER | Full Literal Sequence- |
| | 6218 | TO | 5 | A1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLSYGERLGSPAVS | |
| | | | | | PLPVRGGHVMRGTAFAYVPSPQVLHRIPGTSAYA | |
| | | | | | FPSLGPVALAEHTCPCGEVLERHEPLPAKLALEE | |
| | | | | | EQKPESRLVPKLRQAGAMLLKVPLMLTFLYLFVC | |
| | | | | | SLDMLSSAFQLAGGKVAGDIFKDNAILSNPVAGLVV | |
| | | | | | GILVTVLVQSSSTSTSIIVSMVSSGLLEVSSAIPII | |
| | | | | | MGSNIGTSVTNTIVALMQAGDRTDFRRAFAGATV | |
| | | | | | HDCFNWLSVLVLLPLEAATGYLHHITRLVVASFNI | |
| | | | | | HGGRDAPDLLKIITEPFTKLIIQLDESVITSIATGDE | |
| | | | | | SLRNHSLIQIWCHPDSLQAPTSMSRAEANSSQTL | |
| | | | | | GNATMEKCNHIFVDTGLPDLAVGLILLAGALVLLC | |
| | | | | | TCLILLVKMLNSLLKGQVAKVIQKVINTDFPAPFTW | |
| | | | | | VTGYFAMVVGASMTFVVQSSSVFTSAITPLIGLGV | |
| | | | | | ISIERAYPLTLGSNIGTTTTAILAALASPREKLSSAF | |
| | | | | | QIALCHFFFNISGILLWYPVPCTRLPIRMAKALGKR | |
| | | | | | TAKYRWFAVLYLLVCFLLLPSLVFGISMAGWQVM | |
| | | | | | VGVGTPFGALLAFVVLINVLQSRSPGHLPKWLQT | |
| | | | | | WDFLPRWMHSLKPLDHLITRATLCCARPEPRSPP | |
| | | | | | LPPRVFLEELPPATPSPRLALPAHHNATRL | |
| 158 | JHUO | IPL | 0.31554283 | SLC34 | MLSYGERLGSPAVSPLPVRGGHVMRGTAFAYVP | Core Human ORF Only |
| | 6218 | TO | 5 | A1 | SPQVLHRIPGTSAYAFPSLGPVALAEHTCPCGEV | |
| | | X | | | LERHEPLPAKLALEEEQKPESRLVPKLRQAGAML | |
| | | | | | LKVPLMLTFLYLFVCSLDMLSSAFQLAGGKVAGDI | |
| | | | | | FKDNAILSNPVAGLVVGILVTVLVQSSSTSTSIIVS | |
| | | | | | MVSSGLLEVSSAIPIIMGSNIGTSVTNTIVALMQAG | |
| | | | | | DRTDFRRAFAGATVHDCFNWLSVLVLLPLEAATGYL | |
| | | | | | HHITRLVVASFNIHGGRDAPDLLKIITEPFTKLIIQ | |
| | | | | | LDESVITSIATGDESLRNHSLIQIWCHPDSLQAPTS | |
| | | | | | MSRAEANSSQTLGNATMEKCNHIFVDTGLPDLAV | |
| | | | | | GLILLAGALVLLCTCLILLVKMLNSLLKGQVAKVIQ | |
| | | | | | KVINTDFPAPFTVWTGYFAMVVGASMTFVVQSSSVF | |
| | | | | | TSAITPLIGLGVISIERAYPLTLGSNIGTTTTAILA | |
| | | | | | ALASPREKLSSAFQIALCHFFFNISGILLWYPVPCT | |
| | | | | | RLPIRMAKALGKRTAKYRWFAVLYLLVCFLLLPSL | |
| | | | | | VFGISMAGWQVMVGVGTPFGALLAFVVLINVLQS | |
| | | | | | RSPGHLPKWLQTWDFLPRWMHSLKPLDHLITRA | |
| | | | | | TLCCARPEPRSPPLPPRVFLEELPPATPSPRLALP | |
| | | | | | AHHNATRL | |
| 159 | JHUO | IPL | 0.25427768 | TRNAU | MSPILGYWKIKGLVQPTRLLLEYLEEKYEEHLYER | Full Literal Sequence- |
| | 6702 | TO | 2 | 1AP | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLYEFFVKVYPSCR | |
| | | | | | GGKVVLDQTGVSKGYGFVKFTDELEQKRALTEC | |
| | | | | | QGAVGLGSKPVRLSVAIPKASRVKPVEYSQMYSY | |
| | | | | | SYNQYYQQYQNYYAQWGYDQNTGSYSYSYPQY | |
| | | | | | GYTQSTMQTYEEVGDDALEDPMPQLDVT EANKE | |
| | | | | | FMEQSEELYDALMDCHWQPLDTVSSEIPAMM | |
| 160 | JHUO | IPL | 0.25427768 | TRNAU | MLYEFFVKVYPSCRGGKVVLDQTGVSKGYGFVK | Core Human ORF Only |
| | 6702 | TO | 2 | 1AP | FTDELEQKRALTECQGAVGLGSKPVRLSVAIPKA | |
| | | X | | | SRVKPVEYSQMYSYSYNQYYQQYQNYYAQWGY | |
| | | | | | DQNTGSYSYSYPQYGYTQSTMQTYEEVGDDALE | |
| | | | | | DPMPQLDVTEANKEFMEQSEELYDALMDCHWQ | |
| | | | | | PLDTVSSEIPAMM | |
| 161 | JHUO | IPL | 0.45154611 | KJ9032 | MSPILGYWKIKGLVQPTRLLLEYLEEKYEEHLYER | Full Literal Sequence- |
| | 6904 | TO | 5 | 38 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDLVQVFHCHFNSN | |
| | | | | | FFPLFLEHLLSDHISLSTGSILLGLSYVAPHLNNLY | |
| | | | | | FLSLCRRWGVYAVVRGLFLLKLGLSLLMLLAGPD | |
| | | | | | HLSLLCLFIASNRVFTEGTCKLLTLVVTDLVDEDLV | |
| | | | | | LNHRKQAASALLFGMVALVTKPGQTFAPLLGTWL | |
| | | | | | LCFYTGHDLFQQSLITPVGSAHPWPEPPAPAPAQ | |
| | | | | | APTLRQGCFYLLVLVPITCALLQLFTWSQSTLHGR | |
| | | | | | RLHMVKAQRQNLSQAQTLDVKMV | |
| 162 | JHUO | IPL | 0.45154611 | KJ9032 | MDLVQVFHCHFNSNFFPLFLEHLLSDHISLSTGSI | Core Human ORF Only |
| | 6904 | TO | 5 | 38 | LLGLSYVAPHLNNLYFLSLCRRWGVYAVVRGLFL | |
| | | X | | | LKLGLSLLMLLAGPDHLSLLCLFIASNRVFTEGTC | |
| | | | | | KLLTLVVTDLVDEDLVLNHRKQAASALLFGMVALV | |
| | | | | | TKPGQTFAPLLGTWLLCFYTGHDLFQQSLITPVG | |
| | | | | | SAHPWPEPPAPAPAQAPTLRQGCFYLLVLVPITC | |
| | | | | | ALLQLFTWSQSTLHGRRLHMVKAQRQNLSQAQT | |
| | | | | | LDVKMV | |
| 163 | JHUO | IPL | 0.25015880 | RAB27 | MSPILGYWKIKGLVQPTRLLLEYLEEKYEEHLYER | Full Literal Sequence- |
| | 7273 | TO | 9 | A | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSDGDYDYLIKFLAL | |
| | | | | | GDSGVGKTSVLYQYTDGKFNSKFITTVGIDFREK | |
| | | | | | RVVYRASGPDGATGRGQRIHLQLWDTAGQERFR | |
| | | | | | SLTTAFFRDAMGFLLLFDLTNEQSFLNVRNWISQL | |
| | | | | | QMHAYCENPDIVLCGNKSDLEDQRVVKEEEAIAL | |
| | | | | | AEKYGIPYFETSAANGTNISQAIEMLLDLIMKRME | |
| | | | | | RCVDKSWIPEGVVRSNGHASTDQLSEEKEKGAC | |
| | | | | | GC | |
| 164 | JHUO | IPL | 0.25015880 | RAB27 | MSDGDYDYLIKFLALGDSGVGKTSVLYQYTDGKF | Core Human ORF Only |
| | 7273 | TO | 9 | A | NSKFITTVGIDFREKRVVYRASGPDGATGRGQRI | |
| | | X | | | HLQLWDTAGQERFRSLTTAFFRDAMGFLLLFDLT | |
| | | | | | NEQSFLNVRNWISQLQMHAYCENPDIVLCGNKS | |
| | | | | | DLEDQRVVKEEEAIALAEKYGIPYFETSAANGTNI | |
| | | | | | SQAIEMLLDLIMKRMERCVDKSWIPEGVVRSNGH | |
| | | | | | ASTDQLSEEKEKGACGC | |
| 165 | JHUO | IPL | 0.29217415 | CCNG2 | MSPILGYWKIKGLVQPTRLLLEYLEEKYEEHLYER | Full Literal Sequence- |
| | 7981 | TO | 8 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKDLGAEHLAGHEG | |
| | | | | | VQLLGLLNVYLEQEERFQPREKGLSLIEATPENDN | |
| | | | | | TLCPGLRNAKVEDLRSLANFFGSCTETFVLAVNIL | |
| | | | | | DRFLALMKVKPKHLSCIGVCSFLLAARIVEEDCNIP | |
| | | | | | STHDVIRISQCKCTASDIKRMEKIISEKLHYELEAT | |
| | | | | | TALNFLHLYHTIILCHTSERKEILSLDKLEAQLKAC | |
| | | | | | NCRLIFSKAKPSVLALCLLNLEVETLKSVELLEILLL | |
| | | | | | VKKHSKINDTEFFYWRELVSKCLAEYSSPECCKP | |
| | | | | | DLKKLVWIVSRRTAQNLHNSYYSVPELPTIPEGG | |
| | | | | | CFDESESSVAQAGVQWPDLSSFQPPPTRFKRFS | |
| | | | | | CLSLRSSWDYSSWT | |
| 166 | JHUO | IPL | 0.29217415 | CCNG2 | MKDLGAEHLAGHEGVQLLGLLNVYLEQEERFQP | Core Human ORF Only |
| | 7981 | TO | 8 | | REKGLSLIEATPENDNTLCPGLRNAKVEDLRSLAN | |
| | | X | | | FFGSCTETFVLAVNILDRFLALMKVKPKHLSCIGV | |
| | | | | | CSFLLAARIVEEDCNIPSTHDVIRISQCKCTASDIK | |
| | | | | | RMEKIISEKLHYELEATTALNFLHLYHTIILCHTSER | |
| | | | | | KEILSLDKLEAQLKACNCRLIFSKAKPSVLALCLLN | |
| | | | | | LEVETLKSVELLEILLLVKKHSKINDTEFFYWRELV | |
| | | | | | SKCLAEYSSPECCKPDLKKLVWIVSRRTAQNLHN | |
| | | | | | SYYSVPELPTIPEGGCFDESESSVAQAGVQWPDL | |
| | | | | | SSFQPPPTRFKRFSCLSLRSSWDYSSWT | |
| 167 | JHUO | IPL | 0.02706151 | BDNF | MSPILGYWKIKGLVQPTRLLLEYLEEKYEEHLYER | Full Literal Sequence- |
| | 8454 | TO | 5 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTILFLTMVISYFGCM | |
| | | | | | KAAPMKEANIRGQGGLAYPGVRTHGTLESVNGP | |
| | | | | | KAGSRGLTSLADTFEHVIEELLDEDQKVRPNEEN | |
| | | | | | NKDADLYTSRVMLSSQVPLEPPLLFLLEEYKNYLD | |
| | | | | | AANMSMRVRRHSDPARRGELSVCDSISEVWTAA | |
| | | | | | DKKTAVDMSGGTVTVLEKVPVSKGQLKQYFYET | |
| | | | | | KCNPMGYTKEGCRGIDKRHWNSQCRTTQSYVR | |
| | | | | | ALTMDSKKRIGWRFIRIDTSCVCTLTIKRGR | |
| 168 | JHUO | IPL | 0.02706151 | BDNF | MTILFLTMVISYFGCMKAAPMKEANIRGQGGLAY | Core Human ORF Only |
| | 8454 | TO | 5 | | PGVRTHGTLESVNGPKAGSRGLTSLADTFEHVIE | |
| | | X | | | ELLDEDQKVRPNEENNKDADLYTSRVMLSSQVPL | |
| | | | | | EPPLLFLLEEYKNYLDAANMSMRVRRHSDPARR | |
| | | | | | GELSVCDSISEVWTAADKKTAVDMSGGTVTVLEK | |
| | | | | | VPVSKGQLKQYFYETKCNPMGYTKEGCRGIDKR | |
| | | | | | HWNSQCRTTQSYVRALTMDSKKRIGWRFIRIDTS | |
| | | | | | CVCTLTIKRGR | |
| 169 | JHUO | IPL | 0.62376636 | BOP1 | MSPILGYWKIKGLVQPTRLLLEYLEEKYEEHLYER | Full Literal Sequence- |
| | 8549 | TO | 9 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGSRGAGRTAAPS | |
| | | | | | VRPEKRRSEPELEPEPEPEPPLLCTSPLSHSTGS | |
| | | | | | DSGVSDSEESVFSGLEDSGSDSSEDDDEGDEEG | |
| | | | | | EDGALDDEGHSGIKKTTEEQVQASTPCPRTEMA | |
| | | | | | SARIGDEYAEDSSDEEDIRNTVGNVPLEWYDDFP | |
| | | | | | HVGYDLDGRRIYKPLRTRDELDQFLDKMDDPDY | |
| | | | | | WRTVQDPMTGRDLRLTDEQVALVRRLQSGQFG | |
| | | | | | DVGFNPYEPAVDFFSGDVMIHPVTNRPADKRSFI | |
| | | | | | PSLVEKEKVSRMVHAIKMGWIQPRRPRDPTPSFY | |
| | | | | | DLWAQEDPNAVLGRHKMHVPAPKLALPGHAESY | |
| | | | | | NPPPEYLLSEEERLAWEQQEPGERKLSFLPRKFP | |
| | | | | | SLRAVPAYGRFIQERFERCLDLYLCPRQRKMRVN | |
| | | | | | VDPEDLIPKLPRPRDLQPFPTCQALVYRGHSDLV | |
| | | | | | RCLSVSPGGQWLVSGSDDGSLRLWEVATARCV | |
| | | | | | RTVPVGGVVKSVAWNPSPAVCLVAAAVEDSVLLL | |
| | | | | | NPALGDRLVAGSTDQLLSAFVPPEEPPLQPARWL | |
| | | | | | EASEEERQVGLRLRICHGKPVTQVTWHGRGDYL | |
| | | | | | AVVLATQGHTQVLIHQLSRRRSQSPFRRSHGQV | |
| | | | | | QRVAFHPARPFLLVASQRSVRLYHLLRQELTKKL | |
| | | | | | MPNCKVWSSLAVHPAGDNVICGSYDSKLVWFDL | |
| | | | | | DLSTKPYRMLRHHKKALRAVAFHPRYPLFASGSD | |
| | | | | | DGSVIVCHGMVYNDLLQNPLLVPVKVLKGHVLTR | |
| | | | | | DLGVLDVIFHPTQPVWFSSGADGTVRLFT | |
| 170 | JHUO | IPI_ | 0.62376636 | BOP1 | MAGSRGAGRTAAPSVRPEKRRSEPELEPEPEPE | Core Human ORF Only |
| | 8549 | TO | 9 | | PPLLCTSPLSHSTGSDSGVSDSEESVFSGLEDSG | |
| | | X | | | SDSSEDDDEGDEEGEDGALDDEGHSGIKKTTEE | |
| | | | | | QVQASTPCPRTEMASARIGDEYAEDSSDEEDIRN | |
| | | | | | TVGNVPLEWYDDFPHVGYDLDGRRIYKPLRTRD | |
| | | | | | ELDQFLDKMDDPDYWRTVQDPMTGRDLRLTDE | |
| | | | | | QVALVRRLQSGQFGDVGFNPYEPAVDFFSGDVM | |
| | | | | | IHPVTNRPADKRSFIPSLVEKEKVSRMVHAIKMG | |
| | | | | | WIQPRRPRDPTPSFYDLWAQEDPNAVLGRHKMH | |
| | | | | | VPAPKLALPGHAESYNPPPEYLLSEEERLAWEQQ | |
| | | | | | EPGERKLSFLPRKFPSLRAVPAYGRFIQERFERC | |
| | | | | | LDLYLCPRQRKMRVNVDPEDLIPKLPRPRDLQPF | |
| | | | | | PTCQALVYRGHSDLVRCLSVSPGGQWLVSGSDD | |
| | | | | | GSLRLWEVATARCVRTVPVGGVVKSVAWNPSPA | |
| | | | | | VCLVAAAVEDSVLLLNPALGDRLVAGSTDQLLSA | |
| | | | | | FVPPEEPPLQPARWLEASEEERQVGLRLRICHGK | |
| | | | | | PVTQVTWHGRGDYLAVVLATQGHTQVLIHQLSR | |
| | | | | | RRSQSPFRRSHGQVQRVAFHPARPFLLVASQRS | |
| | | | | | VRLYHLLRQELTKKLMPNCKWVSSLAVHPAGDN | |
| | | | | | VICGSYDSKLVWFDLDLSTKPYRMLRHHKKALRA | |
| | | | | | VAFHPRYPLFASGSDDGSVIVCHGMVYNDLLQNP | |
| | | | | | LLVPVKVLKGHVLTRDLGVLDVIFHPTQPWVFSS | |
| | | | | | GADGTVRLFT | |
| 171 | JHUO | IPI_ | 0.36673236 | ASB6 | MSPILGYWKIKGLVQPTRLLLEYLEEKYEEHLYER | Full Literal Sequence- |
| | 9984 | TO | 4 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPFLHGFRRIIFEYQ | |
| | | | | | PLVDAILGSLGIQDPERQESLDRPSYVASEESRIL | |
| | | | | | VLTELLERKAHSPFYQEGVSNALLKMAELGLTRA | |
| | | | | | ADVLLRHGANLNFEDPVTYYTALHIAVLRNQPDM | |
| | | | | | VELLVHHGADVNRRDRIHESSPLDLASEEPERLP | |
| | | | | | CLQRLLDLGADVNAADKHGKTALLHALASSDGVQ | |
| | | | | | IHNTENIRLLLEGGADVKATTKDGDTVFTCIIFLLG | |
| | | | | | ETVGGDKEEAQMINRFCFQVTRLLLAHGADPSEC | |
| | | | | | PAHESLTHICLKSFKLHFPLLRFLLESGAAYNCSL | |
| | | | | | HGASCWSGFHIIFERLCSHPGCTEDESHADLLRK | |
| | | | | | AETVLDLMVTNSQKLQLPENFDIHPVGSLAEKIQA | |
| | | | | | LHFSLRQLESYPPPLKHLCRVAIRLYLQPWPVDV | |
| | | | | | KVKALPLPDRLKWYLLSEHSGSVEDDI | |
| 172 | JHUO | IPI_ | 0.36673236 | ASB6 | MPFLHGFRRIIFEYQPLVDAILGSLGIQDPERQESL | Core Human ORF Only |
| | 9984 | TO | 4 | | DRPSYVASEESRILVLTELLERKAHSPFYQEGVSN | |
| | | X | | | ALLKMAELGLTRAADVLLRHGANLNFEDPVTYYT | |
| | | | | | ALHIAVLRNQPDMVELLVHHGADVNRRDRIHESS | |
| | | | | | PLDLASEEPERLPCLQRLLDLGADVNAADKHGKT | |
| | | | | | ALLHALASSDGVQIHNTENIRLLLEGGADVKATTK | |
| | | | | | DGDTVFTCIIFLLGETVGGDKEEAQMINRFCFQVT | |
| | | | | | RLLLAHGADPSECPAHESLTHICLKSFKLHFPLLR | |
| | | | | | FLLESGAAYNCSLHGASCWSGFHIIFERLCSHPG | |
| | | | | | CTEDESHADLLRKAETVLDLMVTNSQKLQLPENF | |
| | | | | | DIHPVGSLAEKIQALHFSLRQLESYPPPLKHLCRV | |
| | | | | | AIRLYLQPWPVDVKVKALPLPDRLKWYLLSEHSG | |
| | | | | | SVEDDI | |
| 173 | JHU1 | IPI_ | 0.06534791 | LONRF | MSPILGYWKIKGLVQPTRLLLEYLEEKYEEHLYER | Full Literal Sequence- |
| | 0321 | TO | 8 | 2 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKNYEQALQDASAA | |
| | | | | | CQNEPLLIKGHQVKAQALSGLGRSKEVLKEFLYC | |
| | | | | | LALNPECNSVKKEAQKVMCEVLFSATANVHENLT | |
| | | | | | SSIQSRLKAQGHSHMNAQALLEEGDAGSSENSS | |
| | | | | | EKSDMLGNTNSSVLYFILGLHFEEDKKALESILPT | |
| | | | | | APSAGLKRQFPDDVEDAPDLNAPGKIPKKDLSPQ | |
| | | | | | RSPNSETEESQGLSLDVTDFECALCMRLLFEPVT | |
| | | | | | TPCGHTFCLKCLERCLDHAPHCPLCKDKLSELLA | |
| | | | | | SRNFNITVLAEELIFRYLPDELSDRKRIYDEEMSEL | |
| | | | | | SNLTRDVPIFVCAMAFPTVPCPLHVFEPRYRLMIR | |
| | | | | | RCMETGTKRFGMCLSAEHAGLSEYGCMLEIKDV | |
| | | | | | RTFPDGSSVVDAIGISRFRVLSHRHRDGYNTADIE | |
| | | | | | YLEDEKVEGPEYEELAALHDSVHQQSVSWFASL | |
| | | | | | QDRMKEQILSHFGVMPDREPEPQSNPSGPAWS | |
| | | | | | VWVILAVLPLERKAQLAILGMTSLKERLLAIRRI | |
| | | | | | LVIITRKMNSRQELANARERNN | |
| 174 | JHU1 | IPI_ | 0.06534791 | LONRF | MKNYEQALQDASAACQNEPLLIKGHQVKAQALS | Core Human ORF Only |
| | 0321 | TO | 8 | 2 | GLGRSKEVLKEFLYCLALNPECNSVKKEAQKVMC | |
| | | X | | | EVLFSATANVHENLTSSIQSRLKAQGHSHMNAQA | |
| | | | | | LLEEGDAGSSENSSEKSDMLGNTNSSVLYFILGL | |
| | | | | | HFEEDKKALESILPTAPSAGLKRQFPDDVEDAPDL | |
| | | | | | NAPGKIPKKDLSPQRSPNSETEESQGLSLDVTDF | |
| | | | | | ECALCMRLLFEPVTTPCGHTFCLKCLERCLDHAP | |
| | | | | | HCPLCKDKLSELLASRNFNITVLAEELIFRYLPDEL | |
| | | | | | SDRKRIYDEEMSELSNLTRDVPIFVCAMAFPTVPC | |
| | | | | | PLHVFEPRYRLMIRRCMETGTKRFGMCLSAEHA | |
| | | | | | GLSEYGCMLEIKDVRTFPDGSSVVDAIGISRFRVL | |
| | | | | | SHRHRDGYNTADIEYLEDEKVEGPEYEELAALHD | |
| | | | | | SVHQQSVSWFASLQDRMKEQILSHFGVMPDREP | |
| | | | | | EPQSNPSGPAWSVWVILAVLPLERKAQLAILGMTS | |
| | | | | | LKERLLAIRRILVIITRKMNSRQELANARERNN | |
| 175 | JHU1 | IPL | 0.37768558 | PDXDC | MSPILGYWKIKGLVQPTRLLLEYLEEKYEEHLYER | Full Literal Sequence- |
| | 0332 | TO | 2 | 1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDASLEKIADPTLAE | |
| | | | | | MGKNLKEAVKMLEDSQRRTEEENGKKLISGDIPG | |
| | | | | | PLQGSGQDMVSILQLVQNLMHGDEDEEPQSPRI | |
| | | | | | QNIGEQGHMALLGHSLGAYISTLDKEKLRKLTTRI | |
| | | | | | LSDTTLWLCRIFRYENGCAYFHEEEREGLAKICRL | |
| | | | | | AIHSRYEDFVVDGFNVLYNKKPVIYLSAAARPGLG | |
| | | | | | QYLCNQLGLPFPCLCRVPCNTVFGSQHQMDVAF | |
| | | | | | LEKLIKDDIERGRLPLLLVANAGTAAVGHTDKIGRL | |
| | | | | | KELCEQYGIWLHVEGVNLATLALGYVSSSVLAAA | |
| | | | | | KCDSMTMTPGPWLGLPAVPAVTLYKHDDPALTL | |
| | | | | | VAGLTSNKPTDKLRALPLWLSLQYLGLDGFVERIK | |
| | | | | | HACQLSQRLQESLKEVNYIKILVEDELSSPVVVFR | |
| | | | | | FFQELPGSDPVFKAVPVPNMTPSGVGRERHSCD | |
| | | | | | ALNRWLGEQLKQLVPASGLTVMDLEAEGTCLRF | |
| | | | | | SPLMTAAVLGTRGEDVDQLVACIESKLPVLCCTL | |
| | | | | | QLREEFKQEVEATAGLLYVDDPNWSGIGVVRYE | |
| | | | | | HANDDKSSLKSDPEGENIHAGLLKKLNELESDLTF | |
| | | | | | KIGPEYKSMKSCLYVGMASDNVDAAELVETIAAT | |
| | | | | | AREIEENSRLLENMTEVVRKGIQEAQVELQKASE | |
| | | | | | ERLLEEGVLRQIPVVGSVLNWFSPVQALQKGRTF | |
| | | | | | NLTAGSLESTEPIYVYKAQGAGVTLPPTPSGSRT | |
| | | | | | KQRLPGQKPFKRSLRGSDALSETSSVSHIEDLEK | |
| | | | | | VERLSSGPEQITLEASSTEGHPGAPSPQHTDQTE | |
| | | | | | AFQKGVPHPEDDHSQVEGPESLR | |
| 176 | JHU1 | IPL | 0.37768558 | PDXDC | MDASLEKIADPTLAEMGKNLKEAVKMLEDSQRRT | Core Human ORF Only |
| | 0332 | TO | 2 | 1 | EEENGKKLISGDIPGPLQGSGQDMVSILQLVQNL | |
| | | X | | | MHGDEDEEPQSPRIQNIGEQGHMALLGHSLGAYI | |
| | | | | | STLDKEKLRKLTTRILSDTTLWLCRIFRYENGCAY | |
| | | | | | FHEEEREGLAKICRLAIHSRYEDFVVDGFNVLYNK | |
| | | | | | KPVIYLSAAARPGLGQYLCNQLGLPFPCLCRVPC | |
| | | | | | NTVFGSQHQMDVAFLEKLIKDDIERGRLPLLLVAN | |
| | | | | | AGTAAVGHTDKIGRLKELCEQYGIWLHVEGVNLA | |
| | | | | | TLALGYVSSSVLAAAKCDSMTMTPGPWLGLPAV | |
| | | | | | PAVTLYKHDDPALTLVAGLTSNKPTDKLRALPLWL | |
| | | | | | SLQYLGLDGFVERIKHACQLSQRLQESLKEVNYIK | |
| | | | | | ILVEDELSSPVVVFRFFQELPGSDPVFKAVPVPN | |
| | | | | | MTPSGVGRERHSCDALNRWLGEQLKQLVPASGL | |
| | | | | | TVMDLEAEGTCLRFSPLMTAAVLGTRGEDVDQLV | |
| | | | | | ACIESKLPVLCCTLQLREEFKQEVEATAGLLYVDD | |
| | | | | | PNWSGIGVVRYEHANDDKSSLKSDPEGENIHAGL | |
| | | | | | LKKLNELESDLTFKIGPEYKSMKSCLYVGMASDN | |
| | | | | | VDAAELVETIAATAREIEENSRLLENMTEVVRKGI | |
| | | | | | QEAQVELQKASEERLLEEGVLRQIPVVGSVLNWF | |
| | | | | | SPVQALQKGRTFNLTAGSLESTEPIYVYKAQGAG | |
| | | | | | VTLPPTPSGSRTKQRLPGQKPFKRSLRGSDALSE | |
| | | | | | TSSVSHIEDLEKVERLSSGPEQITLEASSTEGHPG | |
| | | | | | APSPQHTDQTEAFQKGVPHPEDDHSQVEGPESL | |
| | | | | | R | |
| 177 | JHU1 | IPI_ | 0.41449158 | IFNA8 | MSPILGYWKIKGLVQPTRLLLEYLEEKYEEHLYER | Full Literal Sequence- |
| | 0512 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMALTFYLLVALVVLS | |
| | | | | | YKSFSSLGCDLPQTHSLGNRRALILLAQMRRISPF | |
| | | | | | SCLKDRHDFEFPQEEFDDKQFQKAQAISVLHEMI | |
| | | | | | QQTFNLFSTKDSSAALDETLLDEFYIELDQQLNDL | |
| | | | | | ESCVMQEVGVIESPLMYEDSILAVRKYFQRITLYL | |
| | | | | | TEKKYSSCAWEVVRAEIMRSFSLSINLQKRLKSKE | |
| 178 | JHU1 | IPI_ | 0.41449158 | IFNA8 | MALTFYLLVALVVLSYKSFSSLGCDLPQTHSLGN | Core Human ORF Only |
| | 0512 | TO | 1 | | RRALILLAQMRRISPFSCLKDRHDFEFPQEEFDDK | |
| | | X | | | QFQKAQAISVLHEMIQQTFNLFSTKDSSAALDETL | |
| | | | | | LDEFYIELDQQLNDLESCVMQEVGVIESPLMYED | |
| | | | | | SILAVRKYFQRITLYLTEKKYSSCAWEVVRAEIMR | |
| | | | | | SFSLSINLQKRLKSKE | |
| 179 | JHU1 | IPI_ | 0.18120688 | MIR1- | MSPILGYWKIKGLVQPTRLLLEYLEEKYEEHLYER | Full Literal Sequence- |
| | 1240 | TO | 9 | 1HG | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPSCSCALMAPCGP | |
| | | | | | AAGPAAVERTQQVARGEPGSARGQLQVSPEMSI | |
| | | | | | THKEKENAHLKEILLFVNAEAFSQPQPHSAPVCE | |
| | | | | | GQQLTGKFSTSVLTRAGGDASPCSWERLLCYGW | |
| | | | | | SHC | |
| 180 | JHU1 | IPI_ | 0.18120688 | MIR1- | MPSCSCALMAPCGPAAGPAAVERTQQVARGEP | Core Human ORF Only |
| | 1240 | TO | 9 | 1HG | GSARGQLQVSPEMSITHKEKENAHLKEILLFVNAE | |
| | | X | | | AFSQPQPHSAPVCEGQQLTGKFSTSVLTRAGGD | |
| | | | | | ASPCSWERLLCYGWSHC | |
| 181 | JHU1 | IPI_ | 0.09044749 | COLEC | MSPILGYWKIKGLVQPTRLLLEYLEEKYEEHLYER | Full Literal Sequence- |
| | 1732 | TO | 3 | 10 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNGFASLLRRNQFIL | |
| | | | | | LVLFLLQIQSLGLDIDSRPTAEVCATHTISPGPKGD | |
| | | | | | DGEKGDPGEEGKHGKVGRMGPKGIKGELGDMG | |
| | | | | | DQGNIGKTGPIGKKGDKGEKGLLGIPGEKGKAGT | |
| | | | | | VCDCGRYRKFVGQLDISIARLKTSMKFVKNVIAGI | |
| | | | | | RETEEKFYYIVQEEKNYRESLTHCRIRGGMLAMP | |
| | | | | | KDEAANTLIADYVAKSGFFRVFIGVNDLEREGQY | |
| | | | | | MFTDNTPLQNYSNWNEGEPSDPYGHEDCVEML | |
| | | | | | SSGRWNDTECHLTMYFVCEFIKKKK | |
| 182 | JHU1 | IPI_ | 0.09044749 | COLEC | MNGFASLLRRNQFILLVLFLLQIQSLGLDIDSRPTA | Core Human ORF Only |
| | 1732 | TO | 3 | 10 | EVCATHTISPGPKGDDGEKGDPGEEGKHGKVGR | |
| | | X | | | MGPKGIKGELGDMGDQGNIGKTGPIGKKGDKGE | |
| | | | | | KGLLGIPGEKGKAGTVCDCGRYRKFVGQLDISIA | |
| | | | | | RLKTSMKFVKNVIAGIRETEEKFYYIVQEEKNYRE | |
| | | | | | SLTHCRIRGGMLAMPKDEAANTLIADYVAKSGFF | |
| | | | | | RVFIGVNDLEREGQYMFTDNTPLQNYSNWNEGE | |
| | | | | | PSDPYGHEDCVEMLSSGRWNDTECHLTMYFVC | |
| | | | | | EFIKKKK | |
| 183 | JHU1 | IPI_ | 0.60096347 | SIRPA | MSPILGYWKIKGLVQPTRLLLEYLEEKYEEHLYER | Full Literal Sequence- |
| | 2137 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEPAGPAPGRLGPL | |
| | | | | | LCLLLAASCAWSGVAGEEELQVIQPDKSVLVAAG | |
| | | | | | ETATLRCTATSLIPVGPIQWFRGAGPGRELIYNQK | |
| | | | | | EGHFPRVTTVSDLTKRNNMDFSIRIGNITPADAGT | |
| | | | | | YYCVKFRKGSPDDVEFKSGAGTELSVRAKPSAP | |
| | | | | | VVSGPAARATPQHTVSFTCESHGFSPRDITLKWF | |
| | | | | | KNGNELSDFQTNVDPVGESVSYSIHSTAKVVLTR | |
| | | | | | EDVHSQVICEVAHVTLQGDPLRGTANLSETIRVPP | |
| | | | | | TLEVTQQPVRAENQVNVTCQVRKFYPQRLQLTW | |
| | | | | | LENGNVSRTETASTVTENKDGTYNWMSWLLVNV | |
| | | | | | SAHRDDVKLTCQVEHDGQPAVSKSHDLKVSAHP | |
| | | | | | KEQGSNTAAENTGSNERNIYIVVGVVCTLLVALLM | |
| | | | | | AALYLVRIRQKKAQGSTSSTRLHEPEKNAREITQV | |
| | | | | | QSLDTNDITYADLNLPKGKKPAPQAAEPNNHTEY | |
| | | | | | ASIQTSPQPASEDTLTYADLDMVHLNRTPKQPAP | |
| | | | | | KPEPSFSEYASVQVPRK | |
| 184 | JHU1 | IPI_ | 0.60096347 | SIRPA | MEPAGPAPGRLGPLLCLLLAASCAWSGVAGEEE | Core Human ORF Only |
| | 2137 | TO | 1 | | LQVIQPDKSVLVAAGETATLRCTATSLIPVGPIQW | |
| | | X | | | FRGAGPGRELIYNQKEGHFPRVTTVSDLTKRNN | |
| | | | | | MDFSIRIGNITPADAGTYYCVKFRKGSPDDVEFKS | |
| | | | | | GAGTELSVRAKPSAPVVSGPAARATPQHTVSFTC | |
| | | | | | ESHGFSPRDITLKWFKNGNELSDFQTNVDPVGES | |
| | | | | | VSYSIHSTAKVVLTREDVHSQVICEVAHVTLQGDP | |
| | | | | | LRGTANLSETIRVPPTLEVTQQPVRAENQVNVTC | |
| | | | | | QVRKFYPQRLQLTWLENGNVSRTETASTVTENK | |
| | | | | | DGTYNWMSWLLVNVSAHRDDVKLTCQVEHDGQ | |
| | | | | | PAVSKSHDLKVSAHPKEQGSNTAAENTGSNERNI | |
| | | | | | YIVVGVVCTLLVALLMAALYLVRIRQKKAQGSTSS | |
| | | | | | TRLHEPEKNAREITQVQSLDTNDITYADLNLPKGK | |
| | | | | | KPAPQAAEPNNHTEYASIQTSPQPASEDTLTYAD | |
| | | | | | LDMVHLNRTPKQPAPKPEPSFSEYASVQVPRK | |
| 185 | JHU1 | IPL | 0.06943718 | CPSF3 | MSPILGYWKIKGLVQPTRLLLEYLEEKYEEHLYER | Full Literal Sequence- |
| | 2646 | TO | 9 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSAIPAEESDQLLIRP | |
| | | | | | LGAGQEVGRSCIILEFKGRKIMLDCGIHPGLEGMD | |
| | | | | | ALPYIDLIDPAEIDLLLISHFHLDHCGALPWFLQKT | |
| | | | | | SFKGRTFMTHATKAIYRWLLSDYVKVSNISADDM | |
| | | | | | LYTETDLEESMDKIETINFHGVKEVAGIKFWCYHA | |
| | | | | | GHVLGAAMFMIEIAGVKLLYTGDFSRQEDRHLMA | |
| | | | | | AEIPNIKPDILIIESTYGTHIHEKREEREARFCNTVH | |
| | | | | | DIVNRGGRGLIPVFALGRAQELLLILDEYWQNHPE | |
| | | | | | LHDIPIYYASSLAKKCMAVYQTYVNAMNDKIRKQI | |
| | | | | | NINNPFVFKHISNLKSMDHFDDIGPSVVMASPGM | |
| | | | | | MQSGLSRELFESWCTDKRNGVIIAGYCVEGTLAK | |
| | | | | | HIMSEPEEITTMSGQKLPLKMSVDYISFSAHTDYQ | |
| | | | | | QTSEFIRALKPPHVILVHGEQNEMARLKAALIREY | |
| | | | | | EDNDEVHIEVHNPRNTEAVTLNFRGEKLAKVMGF | |
| | | | | | LADKKPEQGQRVSGILVKRNFNYHILSPCDLSNYT | |
| | | | | | DLAMSTVKQTQAIPYTGPFNLLCYQLQKLTGDVE | |
| | | | | | ELEIQEKPALKVFKNITVIQEPGMVVLEWLANPSN | |
| | | | | | DMYADTVTTVILEVQSNPKIRKGAVQKVSKKLEM | |
| | | | | | HVYSKRLEIMLQDIFGEDCVSVKDDSILSVTVDGK | |
| | | | | | TANLNLETRTVECEEGSEDDESLREMVELAAQRL | |
| | | | | | YEALTPVH | |
| 186 | JHU1 | IPL | 0.06943718 | CPSF3 | MSAIPAEESDQLLIRPLGAGQEVGRSCIILEFKGR | Core Human ORF Only |
| | 2646 | TO | 9 | | KIMLDCGIHPGLEGMDALPYIDLIDPAEIDLLLISH | |
| | | X | | | FHLDHCGALPWFLQKTSFKGRTFMTHATKAIYRWL | |
| | | | | | LSDYVKVSNISADDMLYTETDLEESMDKIETINFH | |
| | | | | | GVKEVAGIKFWCYHAGHVLGAAMFMIEIAGVKLLY | |
| | | | | | TGDFSRQEDRHLMAAEIPNIKPDILIIESTYGTHIH | |
| | | | | | EKREEREARFCNTVHDIVNRGGRGLIPVFALGRA | |
| | | | | | QELLLILDEYWQNHPELHDIPIYYASSLAKKCMAV | |
| | | | | | YQTYVNAMNDKIRKQININNPFVFKHISNLKSMDH | |
| | | | | | FDDIGPSVVMASPGMMQSGLSRELFESWCTDKR | |
| | | | | | NGVIIAGYCVEGTLAKHIMSEPEEITTMSGQKLPL | |
| | | | | | KMSVDYISFSAHTDYQQTSEFIRALKPPHVILVHG | |
| | | | | | EQNEMARLKAALIREYEDNDEVHIEVHNPRNTEA | |
| | | | | | VTLNFRGEKLAKVMGFLADKKPEQGQRVSGILVK | |
| | | | | | RNFNYHILSPCDLSNYTDLAMSTVKQTQAIPYTGP | |
| | | | | | FNLLCYQLQKLTGDVEELEIQEKPALKVFKNITVIQ | |
| | | | | | EPGMVVLEWLANPSNDMYADTVTTVILEVQSNPK | |
| | | | | | IRKGAVQKVSKKLEMHVYSKRLEIMLQDIFGEDCV | |
| | | | | | SVKDDSILSVTVDGKTANLNLETRTVECEEGSED | |
| | | | | | DESLREMVELAAQRLYEALTPVH | |
| 187 | JHU1 | IPI_ | 0.12277587 | MYCN | MSPILGYWKIKGLVQPTRLLLEYLEEKYEEHLYER | Full Literal Sequence- |
| | 2779 | TO | 8 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPSCSTSTMPGMIC | |
| | | | | | KNPDLEFDSLQPCFYPDEDDFYFGGPDSTPPGE | |
| | | | | | DIWKKFELLPTPPLSPSRGFAEHSSEPPSVWTEM | |
| | | | | | LLENELWGSPAEEDAFGLGGLGGLTPNPVILQDC | |
| | | | | | MWSGFSAREKLERAVSEKLQHGRGPPTAGSTAQ | |
| | | | | | SPGAGAASPAGRGHGGAAGAGRAGAALPAELA | |
| | | | | | HPAAECVDPAVVFPFPVNKREPAPVPAAPASAPA | |
| | | | | | AGPAVASGAGIAAPAGAPGVAPPRPGGRQTSGG | |
| | | | | | DHKALSTSGEDTLSDSDDEDDEEEDEEEEIDVVT | |
| | | | | | VEKRRSSSNTKAVTTFTITVRPKNAALGPGRAQS | |
| | | | | | SELILKRCLPIHQQHNYAAPSPYVESEDAPPQKKI | |
| | | | | | KSEASPRPLKSVIPPKAKSLSPRNSDSEDSERRR | |
| | | | | | NHNILERQRRNDLRSSFLTLRDHVPELVKNEKAA | |
| | | | | | KVVILKKATEYVHSLQAEEHQLLLEKEKLQARQQ | |
| | | | | | QLLKKIEHARTC | |
| 188 | JHU1 | IPL | 0.12277587 | MYCN | MPSCSTSTMPGMICKNPDLEFDSLQPCFYPDED | Core Human ORF Only |
| | 2779 | TO | 8 | | DFYFGGPDSTPPGEDIWKKFELLPTPPLSPSRGF | |
| | | X | | | AEHSSEPPSWVTEMLLENELWGSPAEEDAFGLG | |
| | | | | | GLGGLTPNPVILQDCMWSGFSAREKLERAVSEKL | |
| | | | | | QHGRGPPTAGSTAQSPGAGAASPAGRGHGGAA | |
| | | | | | GAGRAGAALPAELAHPAAECVDPAVVFPFPVNK | |
| | | | | | REPAPVPAAPASAPAAGPAVASGAGIAAPAGAPG | |
| | | | | | VAPPRPGGRQTSGGDHKALSTSGEDTLSDSDDE | |
| | | | | | DDEEEDEEEEIDVVTVEKRRSSSNTKAVTTFTITV | |
| | | | | | RPKNAALGPGRAQSSELILKRCLPIHQQHNYAAP | |
| | | | | | SPYVESEDAPPQKKIKSEASPRPLKSVIPPKAKSL | |
| | | | | | SPRNSDSEDSERRRNHNILERQRRNDLRSSFLTL | |
| | | | | | RDHVPELVKNEKAAKVVILKKATEYVHSLQAEEH | |
| | | | | | QLLLEKEKLQARQQQLLKKIEHARTC | |
| 189 | JHU1 | IPL | 0.55368128 | COX5A | MSPILGYWKIKGLVQPTRLLLEYLEEKYEEHLYER | Full Literal Sequence- |
| | 2939 | TO | 2 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLGAALRRCAVAATT | |
| | | | | | RADPRGLLHSARTPGPAVAIQSVRCYSHGSQET | |
| | | | | | DEEFDARVWTYFNKPDIDAWELRKGINTLVTYDM | |
| | | | | | VPEPKIIDAALRACRRLNDFASTVRILEVVKDKAG | |
| | | | | | PHKEIYPYVIQELRPTLNELGI STPEELGLDKV | |
| 190 | JHU1 | IPL | 0.55368128 | COX5A | MLGAALRRCAVAATTRADPRGLLHSARTPGPAV | Core Human ORF Only |
| | 2939 | TO | 2 | | AIQSVRCYSHGSQETDEEFDARWVTYFNKPDIDA | |
| | | X | | | WELRKGINTLVTYDMVPEPKIIDAALRACRRLNDF | |
| | | | | | ASTVRILEVVKDKAGPHKEIYPYVIQELRPTLNELG | |
| | | | | | ISTPEELGLDKV | |
| 191 | JHU1 | IPL | 0.00569575 | ITGA5 | MSPILGYWKIKGLVQPTRLLLEYLEEKYEEHLYER | Full Literal Sequence- |
| | 3153 | TO | 6 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGSRTPESPLHAVQ | |
| | | | | | LRWGPRRRPPLLPLLLLLLPPPPRVGGFNLDAEA | |
| | | | | | PAVLSGPPGSFFGFSVEFYRPGTDGVSVLVGAPK | |
| | | | | | ANTSQPGVLQGGAVYLCPWGASPTQCTPIEFDS | |
| | | | | | KGSRLLESSLSSSEGEEPVEYKSLQWFGATVRA | |
| | | | | | HGSSILACAPLYSWRTEKEPLSDPVGTCYLSTDN | |
| | | | | | FTRILEYAPCRSDFSWAAGQGYCQGGFSAEFTK | |
| | | | | | TGRVVLGGPGSYFWQGQILSATQEQIAESYYPEY | |
| | | | | | LINLVQGQLQTRQASSIYDDSYLGYSVAVGEFSG | |
| | | | | | DDTEDFVAGVPKGNLTYGYVTILNGSDIRSLYNFS | |
| | | | | | GEQMASYFGYAVAATDVNGDGLDDLLVGAPLLM | |
| | | | | | DRTPDGRPQEVGRVYVYLQHPAGIEPTPTLTLTG | |
| | | | | | HDEFGRFGSSLTPLGDLDQDGYNDVAIGAPFGG | |
| | | | | | ETQQGVVFVFPGGPGGLGSKPSQVLQPLWAASH | |
| | | | | | TPDFFGSALRGGRDLDGNGYPDLIVGSFGVDKAV | |
| | | | | | VYRGRPIVSASASLTIFPAMFNPEERSCSLEGNPV | |
| | | | | | ACINLSFCLNASGKHVADSIGFTVELQLDWQKQK | |
| | | | | | GGVRRALFLASRQATLTQTLLIQNGAREDCREMK | |
| | | | | | IYLRNESEFRDKLSPIHIALNFSLDPQAPVDSHGLR | |
| | | | | | PALHYQSKSRIEDKAQILLDCGEDNICVPDLQLEV | |
| | | | | | FGEQNHVYLGDKNALNLTFHAQNVGEGGAYEAE | |
| | | | | | LRVTAPPEAEYSGLVRHPGNFSSLSCDYFAVNQS | |
| | | | | | RLLVCDLGNPMKAGASLWGGLRFTVPHLRDTKK | |
| | | | | | TIQFDFQILSKNLNNSQSDVVSFRLSVEAQAQVTL | |
| | | | | | NGVSKPEAVLFPVSDWHPRDQPQKEEDLGPAVH | |
| | | | | | HVYELINQGPSSISQGVLELSCPQALEGQQLLYVT | |
| | | | | | RVTGLNCTTNHPINPKGLELDPEGSLHHQQKREA | |
| | | | | | PSRSSASSGPQILKCPEAECFRLRCELGPLHQQE | |
| | | | | | SQSLQLHFRVWAKTFLQREHQPFSLQCEAVYKA | |
| | | | | | LKMPYRILPRQLPQKERQVATAVQWTKAEGSYG | |
| | | | | | VPLWIIILAILFGLLLLGLLIYILYKLGFFKRSL | |
| | | | | | PYGTAMEKAQLKPPATSDA | |
| 192 | JHU1 | IPI_ | 0.00569575 | ITGA5 | MGSRTPESPLHAVQLRWGPRRRPPLLPLLLLLLP | Core Human ORF Only |
| | 3153 | TO | 6 | | PPPRVGGFNLDAEAPAVLSGPPGSFFGFSVEFYR | |
| | | X | | | PGTDGVSVLVGAPKANTSQPGVLQGGAVYLCPW | |
| | | | | | GASPTQCTPIEFDSKGSRLLESSLSSSEGEEPVE | |
| | | | | | YKSLQWFGATVRAHGSSILACAPLYSWRTEKEPL | |
| | | | | | SDPVGTCYLSTDNFTRILEYAPCRSDFSWAAGQG | |
| | | | | | YCQGGFSAEFTKTGRVVLGGPGSYFWQGQILSA | |
| | | | | | TQEQIAESYYPEYLINLVQGQLQTRQASSIYDDSY | |
| | | | | | LGYSVAVGEFSGDDTEDFVAGVPKGNLTYGYVTI | |
| | | | | | LNGSDIRSLYNFSGEQMASYFGYAVAATDVNGD | |
| | | | | | GLDDLLVGAPLLMDRTPDGRPQEVGRVYVYLQH | |
| | | | | | PAGIEPTPTLTLTGHDEFGRFGSSLTPLGDLDQD | |
| | | | | | GYNDVAIGAPFGGETQQGVVFVFPGGPGGLGSK | |
| | | | | | PSQVLQPLWAASHTPDFFGSALRGGRDLDGNGY | |
| | | | | | PDLIVGSFGVDKAVVYRGRPIVSASASLTIFPAMF | |
| | | | | | NPEERSCSLEGNPVACINLSFCLNASGKHVADSI | |
| | | | | | GFTVELQLDWQKQKGGVRRALFLASRQATLTQT | |
| | | | | | LLIQNGAREDCREMKIYLRNESEFRDKLSPIHIALN | |
| | | | | | FSLDPQAPVDSHGLRPALHYQSKSRIEDKAQILLD | |
| | | | | | CGEDNICVPDLQLEVFGEQNHVYLGDKNALNLTF | |
| | | | | | HAQNVGEGGAYEAELRVTAPPEAEYSGLVRHPG | |
| | | | | | NFSSLSCDYFAVNQSRLLVCDLGNPMKAGASLW | |
| | | | | | GGLRFTVPHLRDTKKTIQFDFQILSKNLNNSQSDV | |
| | | | | | VSFRLSVEAQAQVTLNGVSKPEAVLFPVSDWHP | |
| | | | | | RDQPQKEEDLGPAVHHVYELINQGPSSISQGVLE | |
| | | | | | LSCPQALEGQQLLYVTRVTGLNCTTNHPINPKGL | |
| | | | | | ELDPEGSLHHQQKREAPSRSSASSGPQILKCPEA | |
| | | | | | ECFRLRCELGPLHQQESQSLQLHFRVWAKTFLQ | |
| | | | | | REHQPFSLQCEAVYKALKMPYRILPRQLPQKERQ | |
| | | | | | VATAVQWTKAEGSYGVPLWIIILAILFGLLLLGLL | |
| | | | | | IYILYKLGFFKRSLPYGTAMEKAQLKPPATSDA | |
| 193 | JHU1 | IPL | 0.75139381 | MAX | MSPILGYWKIKGLVQPTRLLLEYLEEKYEEHLYER | Full Literal Sequence- |
| | 3538 | TO | 3 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSDNDDIEVESDEE | |
| | | | | | QPRFQSAADKRAHHNALERKRRDHIKDSFHSLR | |
| | | | | | DSVPSLQGEKLYFLFWKLCTPVLHRQSLMQKCH | |
| | | | | | TFISSYQVHKKKECKI | |
| 194 | JHU1 | IPL | 0.75139381 | MAX | MSDNDDIEVESDEEQPRFQSAADKRAHHNALER | Core Human ORF Only |
| | 3538 | TO | 3 | | KRRDHIKDSFHSLRDSVPSLQGEKLYFLFWKLCT | |
| | | X | | | PVLHRQSLMQKCHTFISSYQVHKKKECKI | |
| 195 | JHU1 | IPL | 0.01765539 | NDRG3 | MSPILGYWKIKGLVQPTRLLLEYLEEKYEEHLYER | Full Literal Sequence- |
| | 3543 | TO | 6 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDELQDVQLTEIKPL | |
| | | | | | LNDKNGTRNFQDFDCQEHDIETTHGVVHVTIRGL | |
| | | | | | PKGNRPVILTYHDIGLNHKSCFNAFFNFEDMQEIT | |
| | | | | | QHFAVCHVDAPGQQEGAPSFPTGYQYPTMDELA | |
| | | | | | EMLPPVLTHLSLKSIIGIGVGAGAYILSRFALNHPE | |
| | | | | | LVEGLVLINVDPCAKGWIDWAASKLSGLTTNVVDI | |
| | | | | | ILAHHFGQEELQANLDLIQTYRMHIAQDINQDNLQ | |
| | | | | | LFLNSYNGRRDLEIERPILGQNDNKSKTLKCSTLL | |
| | | | | | VVGDNSPAVEAVVECNSRLNPINTTLLKMADCGG | |
| | | | | | LPQVVQPGKLTEAFKYFLQGMGYIPSASMTRLAR | |
| | | | | | SRTHSTSSSLGSGESPFSRSVTSNQSDGTQESC | |
| | | | | | ESPDVLDRHQTMEVSC | |
| 196 | JHU1 | IPI_ | 0.01765539 | NDRG3 | MDELQDVQLTEIKPLLNDKNGTRNFQDFDCQEH | Core Human ORF Only |
| | 3543 | TO | 6 | | DIETTHGVVHVTIRGLPKGNRPVILTYHDIGLNHKS | |
| | | X | | | CFNAFFNFEDMQEITQHFAVCHVDAPGQQEGAP | |
| | | | | | SFPTGYQYPTMDELAEMLPPVLTHLSLKSIIGIGV | |
| | | | | | GAGAYILSRFALNHPELVEGLVLINVDPCAKGWID | |
| | | | | | WAASKLSGLTTNVVDIILAHHFGQEELQANLDLIQ | |
| | | | | | TYRMHIAQDINQDNLQLFLNSYNGRRDLEIERPIL | |
| | | | | | GQNDNKSKTLKCSTLLVVGDNSPAVEAVVECNS | |
| | | | | | RLNPINTTLLKMADCGGLPQVVQPGKLTEAFKYF | |
| | | | | | LQGMGYIPSASMTRLARSRTHSTSSSLGSGESPF | |
| | | | | | SRSVTSNQSDGTQESCESPDVLDRHQTMEVSC | |
| 197 | JHU1 | IPI_ | 0.36028617 | BDNF | MSPILGYWKIKGLVQPTRLLLEYLEEKYEEHLYER | Full Literal Sequence- |
| | 3891 | TO | 2 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMFHQVRRVMTILFLT | |
| | | | | | MVISYFGCMKAAPMKEANIRGQGGLAYPGVRTH | |
| | | | | | GTLESVNGPKAGSRGLTSLADTFEHVIEELLDED | |
| | | | | | QKVRPNEENNKDADLYTSRVMLSSQVPLEPPLLF | |
| | | | | | LLEEYKNYLDAANMSMRVRRHSDPARRGELSVC | |
| | | | | | DSISEVWTAADKKTAVDMSGGTVTVLEKVPVSKG | |
| | | | | | QLKQYFYETKCNPMGYTKEGCRGIDKRHWNSQC | |
| | | | | | RTTQSYVRALTMDSKKRIGWRFIRIDTSCVCTLTI | |
| | | | | | KRGR | |
| 198 | JHU1 | IPI_ | 0.36028617 | BDNF | MFHQVRRVMTILFLTMVISYFGCMKAAPMKEANI | Core Human ORF Only |
| | 3891 | TO | 2 | | RGQGGLAYPGVRTHGTLESVNGPKAGSRGLTSL | |
| | | X | | | ADTFEHVIEELLDEDQKVRPNEENNKDADLYTSR | |
| | | | | | VMLSSQVPLEPPLLFLLEEYKNYLDAANMSMRVR | |
| | | | | | RHSDPARRGELSVCDSISEVWTAADKKTAVDMS | |
| | | | | | GGTVTVLEKVPVSKGQLKQYFYETKCNPMGYTK | |
| | | | | | EGCRGIDKRHWNSQCRTTQSYVRALTMDSKKRI | |
| | | | | | GWRFIRIDTSCVCTLTIKRGR | |
| 199 | JHU1 | IPI_ | 0.40234216 | IFITM3 | MSPILGYWKIKGLVQPTRLLLEYLEEKYEEHLYER | Full Literal Sequence- |
| | 4471 | TO | 7 | | DEGDKWRNKKFELGLEFPNLPYYIDGDVKLTQSMA | (including GST-tag, 6- |
| | | X | | | IIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAFPK | ORF, occasional ‘post |
| | | | | | LVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSHTVQTFFSPVNS | |
| | | | | | GQPPNYEMLKEEHEVAVLGGPHNPAPPTSTVIHI | |
| | | | | | RSETSVPDHVVWSLFNTLFMNPCCLGFIAFAYSV | |
| | | | | | KSRDRKMVGDVTGAQAYASTAKCLNIWALILGIL | |
| | | | | | MTILLIVIPVLIFQAYG | |
| 200 | JHU1 | IPI_ | 0.40234216 | IFITM3 | MSHTVQTFFSPVNSGQPPNYEMLKEEHEVAVLG | Core Human ORF Only |
| | 4471 | TO | 7 | | GPHNPAPPTSTVIHIRSETSVPDHVVWSLFNTLFM | |
| | | X | | | NPCCLGFIAFAYSVKSRDRKMVGDVTGAQAYAST | |
| | | | | | AKCLNIWALILGILMTILLIVIPVLIFQAYG | |
| 201 | JHU1 | IPI_ | 0.41628360 | ANKDD | MSPILGYWKIKGLVQPTRLLLEYLEEKYEEHLYER | Full Literal Sequence- |
| | 5773 | TO | 4 | 1A | DEGDKWRNKKFELGLEFPNLPYYIDGDVKLTQSMA | (including GST-tag, 6- |
| | | X | | | IIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAFP | ORF, occasional ‘post |
| | | | | | KLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNALLLSAWFGHLRI | |
| | | | | | LQILVNSGAKIHCESKDGLTLLHCAAQKGHVPVLA | |
| | | | | | FIMEDLEDVALDHVDKLGRTAFHRAAEHGQLDAL | |
| | | | | | DFLVGSGCDHNVKDKEGNTALHLAAGRGHMAVL | |
| | | | | | QRLVDIGLDLEEQNAEGLTALHSAAGGSHPDCVQ | |
| | | | | | LLLRAGSTVNALTQKNLSCLHYAALSGSEDVSRV | |
| | | | | | LIHAGGCANVVDHQGASPLHLAVRHNFPALVRLLI | |
| | | | | | NSDSDVNAVDNRQQTPLHLAAEHAWQDIADMLLI | |
| | | | | | AGVDLNLRDKQGKTALAVAVRSNHVSLVDMIIKA | |
| | | | | | DRFYRWEKDHPSDPSGKSLSFKQDHRQETQQL | |
| | | | | | RSVLWRLASRYLQPREWKKLAYSWEFTEAHVDA | |
| | | | | | IEQQWTGTRSYQEHGHRMLLIWLHGVATAGENP | |
| | | | | | SKALFEGLVAIGRRDLAENIRKKANAAPSAPRRCT | |
| | | | | | AM | |
| 202 | JHU1 | IPI_ | 0.41628360 | ANKDD | MNALLLSAWFGHLRILQILVNSGAKIHCESKDGLT | Core Human ORF Only |
| | 5773 | TO | 4 | 1A | LLHCAAQKGHVPVLAFIMEDLEDVALDHVDKLGR | |
| | | X | | | TAFHRAAEHGQLDALDFLVGSGCDHNVKDKEGN | |
| | | | | | TALHLAAGRGHMAVLQRLVDIGLDLEEQNAEGLT | |
| | | | | | ALHSAAGGSHPDCVQLLLRAGSTVNALTQKNLSC | |
| | | | | | LHYAALSGSEDVSRVLIHAGGCANVVDHQGASPL | |
| | | | | | HLAVRHNFPALVRLLINSDSDVNAVDNRQQTPLH | |
| | | | | | LAAEHAWQDIADMLLIAGVDLNLRDKQGKTALAV | |
| | | | | | AVRSNHVSLVDMIIKADRFYRWEKDHPSDPSGKS | |
| | | | | | LSFKQDHRQETQQLRSVLWRLASRYLQPREWKK | |
| | | | | | LAYSWEFTEAHVDAIEQQWTGTRSYQEHGHRML | |
| | | | | | LIWLHGVATAGENPSKALFEGLVAIGRRDLAEN | |
| | | | | | IRKKANAAPSAPRRCTAM | |
| 203 | JHU1 | IPL | 0.98942581 | MAGE | MSPILGYWKIKGLVQPTRLLLEYLEEKYEEHLYER | Full Literal Sequence- |
| | 5810 | TO | 7 | B10 | DEGDKWRNKKFELGLEFPNLPYYIDGDVKLTQSMA | (including GST-tag, 6- |
| | | X | | | IIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAFP | ORF, occasional ‘post |
| | | | | | KLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPRGQKSKLRAREK | |
| | | | | | RRQARGGLEDLIDALDILEEEEESPPSASACLKDV | |
| | | | | | FQSSLDGASNNPHGLREAQSTSTSATAASHTRHP | |
| | | | | | EGVNDQMEERPICTQDLEATDSFPRGPVDEKVII | |
| | | | | | LVHYLLYKYQMKEPITKADMLRNVTQMSKSQFPV | |
| | | | | | ILSRASEHLELIFGLDLKEVEPNKHIYVLVNKLD | |
| | | | | | LGCDAKLSDETGVPKTGLLMTVLGIIFTNGNCVA | |
| | | | | | EEEVWKVFNTMGLYDGIEHFMFGEPRKLLTKDLV | |
| | | | | | YKENLEYQQVPNSDPPRYQFLWGPRAHAETSKMK | |
| | | | | | EVLFLAKVNDTAPSEFSNWYTEALQDEEERARAR | |
| | | | | | VAAKARVSATAGARSKVKSSKSSQLQ | |
| 204 | JHU1 | IPL | 0.98942581 | MAGE | MPRGQKSKLRAREKRRQARGGLEDLIDALDILEE | Core Human ORF Only |
| | 5810 | TO | 7 | B10 | EEESPPSASACLKDVFQSSLDGASNNPHGLREA | |
| | | X | | | QSTSTSATAASHTRHPEGVNDQMEERPICTQDLE | |
| | | | | | ATDSFPRGPVDEKVIILVHYLLYKYQMKEPITKAD | |
| | | | | | MLRNVTQMSKSQFPVILSRASEHLELIFGLDLKEV | |
| | | | | | EPNKHIYVLVNKLDLGCDAKLSDETGVPKTGLLM | |
| | | | | | TVLGIIFTNGNCVAEEEVWKVFNTMGLYDGIEHF | |
| | | | | | MFGEPRKLLTKDLVKENYLEYQQVPNSDPPRYQ | |
| | | | | | FLWGPRAHAETSKMKVLEFLAKVNDTAPSEFSN | |
| | | | | | WYTEALQDEEERARARVAAKARVSATAGARSKV | |
| | | | | | KSSKSSQLQ | |
| 205 | JHU1 | IPL | 0.12025512 | TTC6 | MSPILGYWKIKGLVQPTRLLLEYLEEKYEEHLYER | Full Literal Sequence- |
| | 5848 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDKGLSELSPMQQA | |
| | | | | | LIYSFCENHDKAIEVLDGISWNRAEMTMCALLAKV | |
| | | | | | QMKAKRTKEAVEVLKKALDAISHSDKGPDATAISA | |
| | | | | | DCLYNLGLCYMEEGNLQMAFDSFTKAVKANPDF | |
| | | | | | AESFYQRGLCKVKLHKDSSILDFNRAITLNPKHYQ | |
| | | | | | AYLSRVAFYGLKGRYSKAILNCNKAIKIYPESVRA | |
| | | | | | YLYRGVLKYYNKTYKLAITDLTTAISMDKNSYIAFY | |
| | | | | | NRALCYTKIRELQMALTDYGIVLLLDATETVKLNTF | |
| | | | | | LNRGLIYVELGQYGFALEDFKQAALISRTNGSLCH | |
| | | | | | ATAMCHHRINEFEEAVNFFTWALKINPCFLDAYV | |
| | | | | | GRGNSYMEYGHDEATKQAQKDFLKALHINPAYIK | |
| | | | | | ARISFGYNLQAQGKFQKAWNHFTIAIDTDPKNYLA | |
| | | | | | YEGRAVVCLQMGNNFAAMQDINAAMKISTTAEFL | |
| | | | | | TNRGVIHEFMGHKQNAMKDYQDAITLNPKYSLAY | |
| | | | | | FNAGNIYFHHRQFSQASDYFSKALKFDPENEYVL | |
| | | | | | MNRAITNTILKKYEEAKEDFANVIESCPFWAAVYF | |
| | | | | | NRAHFYYCLKQYELAEEDLNKALSLKPNDALVYN | |
| | | | | | FRAKVRGKIGLIEEAMADYNQALDLEDYASVI | |
| 206 | JHU1 | IPL | 0.12025512 | TTC6 | MDKGLSELSPMQQALIYSFCENHDKAIEVLDGIS | Core Human ORF Only |
| | 5848 | TO | 1 | | WNRAEMTMCALLAKVQMKAKRTKEAVEVLKKAL | |
| | | X | | | DAISHSDKGPDATAISADCLYNLGLCYMEEGNLQ | |
| | | | | | MAFDSFTKAVKANPDFAESFYQRGLCKVKLHKDS | |
| | | | | | SILDFNRAITLNPKHYQAYLSRVAFYGLKGRYSKAI | |
| | | | | | LNCNKAIKIYPESVRAYLYRGVLKYYNKTYKLAITD | |
| | | | | | LTTAISMDKNSYIAFYNRALCYTKIRELQMALTDY | |
| | | | | | GIVLLLDATETVKLNTFLNRGLIYVELGQYGFALED | |
| | | | | | FKQAALISRTNGSLCHATAMCHHRINEFEEAVNFF | |
| | | | | | TWALKINPCFLDAYVGRGNSYMEYGHDEATKQA | |
| | | | | | QKDFLKALHINPAYIKARISFGYNLQAQGKFQKAW | |
| | | | | | NHFTIAIDTDPKNYLAYEGRAVVCLQMGNNFAAM | |
| | | | | | QDINAAMKISTTAEFLTNRGVIHEFMGHKQNAMK | |
| | | | | | DYQDAITLNPKYSLAYFNAGNIYFHHRQFSQASD | |
| | | | | | YFSKALKFDPENEYVLMNRAITNTILKKYEEAKED | |
| | | | | | FANVIESCPFWAAVYFNRAHFYYCLKQYELAEED | |
| | | | | | LNKALSLKPNDALVYNFRAKVRGKIGLIEEAMADY | |
| | | | | | NQALDLEDYASVI | |
| 207 | JHU1 | IPI_ | 0.30743442 | CELA1 | MSPILGYWKIKGLVQPTRLLLEYLEEKYEEHLYER | Full Literal Sequence- |
| | 5891 | TO | 9 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLVLYGHSTQDLPET | |
| | | | | | NARVVGGTEAGRNSWPSQISLQYRSGGSRYHTC | |
| | | | | | GGTLIRQNVWMTAAHCVDYQKTFRVVAGDHNLS | |
| | | | | | QNDGTEQYVSVQKIVVHPYWNSDNVAAGYDIALL | |
| | | | | | RLAQSVTLNSYVQLGVLPQEGAILANNSPCYITG | |
| | | | | | WGKTKTNGQLAQTLQQAYLPSVDYAICSSSSYW | |
| | | | | | GSTVKNTMVCAGGDGVRSGCQGDSGGPLHCLV | |
| | | | | | NGKYSVHGVTSFVSSRGCNVSRKPTVFTQVSAYI | |
| | | | | | SWINNVIASN | |
| 208 | JHU1 | IPI_ | 0.30743442 | CELA1 | MLVLYGHSTQDLPETNARVVGGTEAGRNSWPSQ | Core Human ORF Only |
| | 5891 | TO | 9 | | ISLQYRSGGSRYHTCGGTLIRQNVWMTAAHCVD | |
| | | X | | | YQKTFRVVAGDHNLSQNDGTEQYVSVQKIVVHP | |
| | | | | | YWNSDNVAAGYDIALLRLAQSVTLNSYVQLGVLP | |
| | | | | | QEGAILANNSPCYITGWGKTKTNGQLAQTLQQAY | |
| | | | | | LPSVDYAICSSSSYWGSTVKNTMVCAGGDGVRS | |
| | | | | | GCQGDSGGPLHCLVNGKYSVHGVTSFVSSRGC | |
| | | | | | N VSRKPTVFTQVSAYI SWINNVIASN | |
| 209 | JHU1 | IPI_ | 0.53379481 | ACER2 | MSPILGYWKIKGLVQPTRLLLEYLEEKYEEHLYER | Full Literal Sequence- |
| | 5962 | TO | 7 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMCLFRQYATCFNSGI | |
| | | | | | YLIWTLLVVVGIGSVYFHATLSFLGQMLDELAVLW | |
| | | | | | VLMCALAMWFPRRYLPKIFRNDRGRFKVVVSVLS | |
| | | | | | AVTTCLAFVKPAINNISLMTLGVPCTALLIAELKRC | |
| | | | | | DNMRVFKLGLFSGLWWTLALFCWISDRAFCELLS | |
| | | | | | SFNFPYLHCMWHILICLAAYLGCVCFAYFDAASEI | |
| | | | | | PEQGPVIKFWPNEKWAFIGVPYVSLLCANKKSSV | |
| | | | | | KIT | |
| 210 | JHU1 | IPI_ | 0.53379481 | ACER2 | MCLFRQYATCFNSGIYLIWTLLVVVGIGSVYFHAT | Core Human ORF Only |
| | 5962 | TO | 7 | | LSFLGQMLDELAVLWVLMCALAMWFPRRYLPKIF | |
| | | X | | | RNDRGRFKVVVSVLSAVTTCLAFVKPAINNISLMT | |
| | | | | | LGVPCTALLIAELKRCDNMRVFKLGLFSGLVWVTL | |
| | | | | | ALFCWISDRAFCELLSSFNFPYLHCMWHILICLAA | |
| | | | | | YLGCVCFAYFDAASEIPEQGPVIKFWPNEKWAFI | |
| | | | | | GVPYVSLLCANKKSSVKIT | |
| 211 | JHU1 | IPI_ | 0.34009133 | STX16 | MSPILGYWKIKGLVQPTRLLLEYLEEKYEEHLYER | Full Literal Sequence- |
| | 6331 | TO | 8 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMATRRLTDAFLLLRN | |
| | | | | | NSIQNRQLLAEQVSSHITSSPLHSRSIAALADDRM | |
| | | | | | ALVSGISLDPEAAIGVTKRPPPKWVDGVDEIQYDV | |
| | | | | | GRIKQKMKELASLHDKHLNRPTLDDSSEEEHAIEI | |
| | | | | | TTQEITQLFHRCQRAVQALPSRARACSEQEGRLL | |
| | | | | | GNVVASLAQALQELSTSFRHAQSGYLKRMKNRE | |
| | | | | | ERSQHFFDTSVPLMDDGDDNTLYHRGFTEDQLV | |
| | | | | | LVEQNTLMVEEREREIRQIVQSISDLNEIFRDLGA | |
| | | | | | MIVEQGTVLDRIDYNVEQSCIKTEDGLKQLHKAEQ | |
| | | | | | YQKKNRKMLVILILFVIIIVLIVVLVGVKSR | |
| 212 | JHU1 | IPL | 0.34009133 | STX16 | MATRRLTDAFLLLRNNSIQNRQLLAEQVSSHITSS | Core Human ORF Only |
| | 6331 | TO | 8 | | PLHSRSIAALADDRMALVSGISLDPEAAIGVTKRP | |
| | | X | | | PPKWVDGVDEIQYDVGRIKQKMKELASLHDKHLN | |
| | | | | | RPTLDDSSEEEHAIEITTQEITQLFHRCQRAVQAL | |
| | | | | | PSRARACSEQEGRLLGNVVASLAQALQELSTSFR | |
| | | | | | HAQSGYLKRMKNREERSQHFFDTSVPLMDDGD | |
| | | | | | DNTLYHRGFTEDQLVLVEQNTLMVEEREREIRQI | |
| | | | | | VQSISDLNEIFRDLGAMIVEQGTVLDRIDYNVEQS | |
| | | | | | CIKTEDGLKQLHKAEQYQKKNRKMLVILILFVIII | |
| | | | | | VVLIVLVGVKSR | |
| 213 | JHU1 | IPL | 0.52713864 | IGL@ | MSPILGYWKIKGLVQPTRLLLEYLEEKYEEHLYER | Full Literal Sequence- |
| | 6435 | TO | 6 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAWSPLLLTLLAHCT | |
| | | | | | GSWAQSVLTQPPSVSGAPGQRVTISCTGSSSNIG | |
| | | | | | AGYDVHWYQQLPGTAPRLLIYGNSNRPSGVPDR | |
| | | | | | FSGSKSGTSASLAITGLQAEDEADYYCQSYDSSL | |
| | | | | | SGVMFGGGTKLTVLGQPKAAPSVTLFPPSSEELQ | |
| | | | | | ANKATLVCLISDFYPGAVTVAWKADSSPVKAGVE | |
| | | | | | TTTPSKQSNNKYAASSYLSLTPEQWKSHRSYSC | |
| | | | | | QVTHEGSTVEKTVAPTECS | |
| 214 | JHU1 | IPL | 0.52713864 | IGL@ | MAWSPLLLTLLAHCTGSWAQSVLTQPPSVSGAP | Core Human ORF Only |
| | 6435 | TO | 6 | | GQRVTISCTGSSSNIGAGYDVHWYQQLPGTAPR | |
| | | X | | | LLIYGNSNRPSGVPDRFSGSKSGTSASLAITGLQA | |
| | | | | | EDEADYYCQSYDSSLSGVMFGGGTKLTVLGQPK | |
| | | | | | AAPSVTLFPPSSEELQANKATLVCLISDFYPGAVT | |
| | | | | | VAWKADSSPVKAGVETTTPSKQSNNKYAASSYL | |
| | | | | | SLTPEQWKSHRSYSCQVTHEGSTVEKTVAPTEC | |
| | | | | | S | |
| 215 | JHU1 | IPL | 0.57683241 | PRTN3 | MSPILGYWKIKGLVQPTRLLLEYLEEKYEEHLYER | Full Literal Sequence- |
| | 6551 | TO | 9 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAHRPPSPALASVLL | |
| | | | | | ALLLSGAARAAEIVGGHEAQPHSRPYMASLQMR | |
| | | | | | GNPGSHFCGGTLIHPSFVLTAAHCLRDIPQRLVN | |
| | | | | | VVLGAHNVRTQEPTQQHFSVAQVFLNNYDAENK | |
| | | | | | LNDILLIQLSSPANLSASVATVQLPQQDQPVPHGT | |
| | | | | | QCLAMGWGRVGAHDPPAQVLQELNVTVVTFFCR | |
| | | | | | PHNICTFVPRRKAGICFGDSGGPLICDGIIQGIDSF | |
| | | | | | VIWGCATRLFPDFFTRVALYVDWIRSTLRRVEAK | |
| | | | | | GRP | |
| 216 | JHU1 | IPL | 0.57683241 | PRTN3 | MAHRPPSPALASVLLALLLSGAARAAEIVGGHEA | Core Human ORF Only |
| | 6551 | TO | 9 | | QPHSRPYMASLQMRGNPGSHFCGGTLIHPSFVL | |
| | | X | | | TAAHCLRDIPQRLVNVVLGAHNVRTQEPTQQHFS | |
| | | | | | VAQVFLNNYDAENKLNDILLIQLSSPANLSASVAT | |
| | | | | | VQLPQQDQPVPHGTQCLAMGWGRVGAHDPPAQ | |
| | | | | | VLQELNVTVVTFFCRPHNICTFVPRRKAGICFGDS | |
| | | | | | GGPLICDGIIQGIDSFVIWGCATRLFPDFFTRVALY | |
| | | | | | VDWIRSTLRRVEAKGRP | |
| 217 | JHU1 | IPL | 0.0047203 | SUCNR | MSPILGYWKIKGLVQPTRLLLEYLEEKYEEHLYER | Full Literal Sequence- |
| | 6570 | TO | 78 | 1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLGIMAWNATCKNWL | |
| | | | | | AAEAALEKYYLSIFYGIEFVVGVLGNTIVVYGYIFS | |
| | | | | | LKNWNSSNIYLFNLSVSDLAFLCTLPMLIRSYANGN | |
| | | | | | WIYGDVLCISNRYVLHANLYTSILFLTFISIDRYLII | |
| | | | | | KYPFREHLLQKKEFAILISLAIWVLVTLELLPILPL | |
| | | | | | INPVITDNGTTCNDFASSGDPNYNLIYSMCLTLLGF | |
| | | | | | LIPLFVMCFFYYKIALFLKQRNRQVATALPLEKPLN | |
| | | | | | LVIMAVVIFSVLFTPYHVMRNVRIASRLGSWKQYQ | |
| | | | | | CTQVVINSFYIVTRPLAFLNSVINPVFYFLLGDHFR | |
| | | | | | DMLMNQLRHNFKSLTSFSRWAHELLLSFREK | |
| 218 | JHU1 | IPL | 0.0047203 | SUCNR | MLGIMAWNATCKNWLAAEAALEKYYLSIFYGIEFV | Core Human ORF Only |
| | 6570 | TO | 78 | 1 | VGVLGNTIVVYGYIFSLKNWNSSNIYLFNLSVSDL | |
| | | X | | | AFLCTLPMLIRSYANGNWIYGDVLCISNRYVLHAN | |
| | | | | | LYTSILFLTFISIDRYLIIKYPFREHLLQKKEFAILISL | |
| | | | | | AIVWLVTLELLPILPLINPVITDNGTTCNDFASSGD | |
| | | | | | PNYNLIYSMCLTLLGFLIPLFVMCFFYYKIALFLKQ | |
| | | | | | RNRQVATALPLEKPLNLVIMAVVIFSVLFTPYHVM | |
| | | | | | RNVRIASRLGSWKQYQCTQVVINSFYIVTRPLAFL | |
| | | | | | NSVINPVFYFLLGDHFRDMLMNQLRHNFKSLTSF | |
| | | | | | SRWAHELLLSFREK | |
| 219 | JHU1 | IPL | 0.21173217 | CSTL1 | MSPILGYWKIKGLVQPTRLLLEYLEEKYEEHLYER | Full Literal Sequence- |
| | 6848 | TO | 8 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGIGCWRNPLLLLIA | |
| | | | | | LVLSAKLGHFQRWEGFQQKLMSKKNMNSTLNFFI | |
| | | | | | QSYNNASNDTYLYRVQRLIRSQMQLTTGVEYIVT | |
| | | | | | VKIGRTKCKRNDTSNSSCPLQSKKLRKSLICESLI | |
| | | | | | YTMPWINYFQLWNNSCLEAEHVGRNLRNPAFLY | |
| | | | | | KVVKASS | |
| 220 | JHU1 | IPL | 0.21173217 | CSTL1 | MGIGCWRNPLLLLIALVLSAKLGHFQRWEGFQQK | Core Human ORF Only |
| | 6848 | TO | 8 | | LMSKKNMNSTLNFFIQSYNNASNDTYLYRVQRLI | |
| | | X | | | RSQMQLTTGVEYIVTVKIGRTKCKRNDTSNSSCP | |
| | | | | | LQSKKLRKSLICESLIYTMPWINYFQLWNNSCLEA | |
| | | | | | EHVGRNLR | |
| 221 | JHU1 | IPL | 0.05532295 | TNFRS | MSPILGYWKIKGLVQPTRLLLEYLEEKYEEHLYER | Full Literal Sequence- |
| | 7198 | TO | 8 | F25 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEQRPRGCAAVAAA | |
| | | | | | LLLVLLGARAQGGTRSPRCDCAGDFHKKIGLFCC | |
| | | | | | RGCPAGHYLKAPCTEPCGNSTCLVCPQDTFLAW | |
| | | | | | ENHHNSECARCQACDEQASQVALENCSAVADTR | |
| | | | | | CGCKPGWFVECQVSQCVSSSPFYCQPCLDCGA | |
| | | | | | LHRHTRLLCSRRDTDCGTCLPGFYEHGDGCVSC | |
| | | | | | PTSTLGSCPERCAAVCGWRQMFWVQVLLAGLVV | |
| | | | | | PLLLGATLTYTYRHCWPHKPLVTADEAGMEALTP | |
| | | | | | PPATHLSPLDSAHTLLAPPDSSEKICTVQLVGNS | |
| | | | | | WTPGYPETQEALCPQVTWSWDQLPSRALGPAA | |
| | | | | | APTLSPESPAGSPAMMLQPGPQLYDVMDAVPAR | |
| | | | | | RWKEFVRTLGLREAEIEAVEVEIGRFRDQQYEML | |
| | | | | | KRWRQQQPAGLGAVYAALERMGLDGCVEDLRS | |
| | | | | | RLQRGPNPAFLYKVVKASS | |
| 222 | JHU1 | IPL | 0.05532295 | TNFRS | MEQRPRGCAAVAAALLLVLLGARAQGGTRSPRC | Core Human ORF Only |
| | 7198 | TO | 8 | F25 | DCAGDFHKKIGLFCCRGCPAGHYLKAPCTEPCG | |
| | | X | | | NSTCLVCPQDTFLAWENHHNSECARCQACDEQA | |
| | | | | | SQVALENCSAVADTRCGCKPGWFVECQVSQCV | |
| | | | | | SSSPFYCQPCLDCGALHRHTRLLCSRRDTDCGT | |
| | | | | | CLPGFYEHGDGCVSCPTSTLGSCPERCAAVCGW | |
| | | | | | RQMFWVQVLLAGLVVPLLLGATLTYTYRHCWPH | |
| | | | | | KPLVTADEAGMEALTPPPATHLSPLDSAHTLLAPP | |
| | | | | | DSSEKICTVQLVGNSWTPGYPETQEALCPQVTW | |
| | | | | | SWDQLPSRALGPAAAPTLSPESPAGSPAMMLQP | |
| | | | | | GPQLYDVMDAVPARRWKEFVRTLGLREAEIEAVE | |
| | | | | | VEIGRFRDQQYEMLKRWRQQQPAGLGAVYAALE | |
| | | | | | RMGLDGCVEDLRSRLQRGP | |
| 223 | JHU1 | IPL | 0.40855453 | TNFRS | MSPILGYWKIKGLVQPTRLLLEYLEEKYEEHLYER | Full Literal Sequence- |
| | 7198 | TO | 9 | F25 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEQRPRGCAAVAAA | |
| | | | | | LLLVLLGARAQGGTRSPRCDCAGDFHKKIGLFCC | |
| | | | | | RGCPAGHYLKAPCTEPCGNSTCLVCPQDTFLAW | |
| | | | | | ENHHNSECARCQACDEQASQVALENCSAVADTR | |
| | | | | | CGCKPGWFVECQVSQCVSSSPFYCQPCLDCGA | |
| | | | | | LHRHTRLLCSRRDTDCGTCLPGFYEHGDGCVSC | |
| | | | | | PTSTLGSCPERCAAVCGWRQMFWVQVLLAGLVV | |
| | | | | | PLLLGATLTYTYRHCWPHKPLVTADEAGMEALTP | |
| | | | | | PPATHLSPLDSAHTLLAPPDSSEKICTVQLVGNS | |
| | | | | | WTPGYPETQEALCPQVTWSWDQLPSRALGPAA | |
| | | | | | APTLSPESPAGSPAMMLQPGPQLYDVMDAVPAR | |
| | | | | | RWKEFVRTLGLREAEIEAVEVEIGRFRDQQYEML | |
| | | | | | KRWRQQQPAGLGAVYAALERMGLDGCVEDLRS | |
| | | | | | RLQRGPNPAFLYKVVKASS | |
| 224 | JHU1 | IPI_ | 0.40855453 | TNFRS | MEQRPRGCAAVAAALLLVLLGARAQGGTRSPRC | Core Human ORF Only |
| | 7198 | TO | 9 | F25 | DCAGDFHKKIGLFCCRGCPAGHYLKAPCTEPCG | |
| | | X | | | NSTCLVCPQDTFLAWENHHNSECARCQACDEQA | |
| | | | | | SQVALENCSAVADTRCGCKPGWFVECQVSQCV | |
| | | | | | SSSPFYCQPCLDCGALHRHTRLLCSRRDTDCGT | |
| | | | | | CLPGFYEHGDGCVSCPTSTLGSCPERCAAVCGW | |
| | | | | | RQMFWVQVLLAGLVVPLLLGATLTYTYRHCWPH | |
| | | | | | KPLVTADEAGMEALTPPPATHLSPLDSAHTLLAPP | |
| | | | | | DSSEKICTVQLVGNSWTPGYPETQEALCPQVTW | |
| | | | | | SWDQLPSRALGPAAAPTLSPESPAGSPAMMLQP | |
| | | | | | GPQLYDVMDAVPARRWKEFVRTLGLREAEIEAVE | |
| | | | | | VEIGRFRDQQYEMLKRWRQQQPAGLGAVYAALE | |
| | | | | | RMGLDGCVEDLRSRLQRGP | |
| 225 | JHU1 | IPL | 0.42586733 | LZTR1 | MSPILGYWKIKGLVQPTRLLLEYLEEKYEEHLYER | Full Literal Sequence- |
| | 9825 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGPGSTGGQIGAA | |
| | | | | | ALAGGARSKVAPSVDFDHSCSDSVEYLTLNFGPF | |
| | | | | | ETVHRWRRLPPCDEFVGARRSKHTVVAYKDAIYV | |
| | | | | | FGGDNGKTMLNDLLRFDVKDCSWCRAFTTGTPP | |
| | | | | | APRYHHSAVVYGSSMFVFGGYTGDIYSNSNLKN | |
| | | | | | KNDLFEYKFATGQWTEWKIEGRLPVARSAHGAT | |
| | | | | | VYSDKLWIFAGYDGNARLNDMWTIGLQDRELTC | |
| | | | | | WEEVAQSGEIPPSCCNFPVAVCRDKMFVFSGQS | |
| | | | | | GAKITNNLFQFEFKDKTWTRIPTEHLLRGSPPPPQ | |
| | | | | | RRYGHTMVAFDRHLYVFGGAADNTLPNELHCYD | |
| | | | | | VDFQTWEVVQPSSDSEVGGAEVPERACASEEVP | |
| | | | | | TLTYEERVGFKKSRDVFGLDFGTTSAKQPTQPAS | |
| | | | | | ELPSGRLFHAAAVISDAMYIFGGTVDNNIRSGEMY | |
| | | | | | RFQFSCYPKCTLHEDYGRLWESRQFCDVEFVLG | |
| | | | | | EKEECVQGHVAIVTARSRWLRRKITQARERLAQK | |
| | | | | | LEQEAAPVPREAPGVAAGGARPPLLHVAIREAEA | |
| | | | | | RPFEVLMQFLYTDKIKYPRKGHVEDVLLIMDVYKL | |
| | | | | | ALSFQLCRLEQLCRQYIEASVDLQNVLVVCESAA | |
| | | | | | RLQLSQLKEHCLNFVVKESHFNQVIMMKEFERLS | |
| | | | | | SPLIVEIVRRKQQPPPRTPLDQPVDIGTSLIQDMK | |
| | | | | | AYLEGAGAEFCDITLLLDGHPRPAHKAILAARSSY | |
| | | | | | FEAMFRSFMPEDGQVNISIGEMVPSRQAFESMLR | |
| | | | | | YIYYGEVNMPPEDSLYLFAAPYYYGFYNNRLQAY | |
| | | | | | CKQNLEMNVTVQNVLQILEAADKTQALDMKRHCL | |
| | | | | | HIIVHQFTKVSKLPTLRSLSQQLLLDIIDSLASHIS | |
| | | | | | DKQCAELGADINPAFLYKVVKASS | |
| 226 | JHU1 | IPL | 0.42586733 | LZTR1 | MAGPGSTGGQIGAAALAGGARSKVAPSVDFDHS | Core Human ORF Only |
| | 9825 | TO | 1 | | CSDSVEYLTLNFGPFETVHRWRRLPPCDEFVGA | |
| | | X | | | RRSKHTWAYKDAIYVFGGDNGKTMLNDLLRFDV | |
| | | | | | KDCSWCRAFTTGTPPAPRYHHSAVVYGSSMFVF | |
| | | | | | GGYTGDIYSNSNLKNKNDLFEYKFATGQWTEWKI | |
| | | | | | EGRLPVARSAHGATVYSDKLWIFAGYDGNARLN | |
| | | | | | DMWTIGLQDRELTCWEEVAQSGEIPPSCCNFPV | |
| | | | | | AVCRDKMFVFSGQSGAKITNNLFQFEFKDKTWT | |
| | | | | | RIPTEHLLRGSPPPPQRRYGHTMVAFDRHLYVFG | |
| | | | | | GAADNTLPNELHCYDVDFQTWEVVQPSSDSEVG | |
| | | | | | GAEVPERACASEEVPTLTYEERVGFKKSRDVFGL | |
| | | | | | DFGTTSAKQPTQPASELPSGRLFHAAAVISDAMYI | |
| | | | | | FGGTVDNNIRSGEMYRFQFSCYPKCTLHEDYGR | |
| | | | | | LWESRQFCDVEFVLGEKEECVQGHVAIVTARSR | |
| | | | | | WLRRKITQARERLAQKLEQEAAPVPREAPGVAA | |
| | | | | | GGARPPLLHVAIREAEARPFEVLMQFLYTDKIKYP | |
| | | | | | RKGHVEDVLLIMDVYKLALSFQLCRLEQLCRQYIE | |
| | | | | | ASVDLQNVLVVCESAARLQLSQLKEHCLNFVVKE | |
| | | | | | SHFNQVIMMKEFERLSSPLIVEIVRRKQQPPPRTP | |
| | | | | | LDQPVDIGTSLIQDMKAYLEGAGAEFCDITLLLDG | |
| | | | | | HPRPAHKAILAARSSYFEAMFRSFMPEDGQVNISI | |
| | | | | | GEMVPSRQAFESMLRYIYYGEVNMPPEDSLYLFA | |
| | | | | | APYYYGFYNNRLQAYCKQNLEMNVTVQNVLQILE | |
| | | | | | AADKTQALDMKRHCLHIIVHQFTKVSKLPTLRSLS | |
| | | | | | QQLLLDIIDSLASHISDKQCAELGADI | |
| 227 | JHU1 | IPI_ | 0.2332162 | PIWIL2 | MSPILGYWKIKGLVQPTRLLLEYLEEKYEEHLYER | Full Literal Sequence- |
| | 9836 | TO | 1 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDPFRPSFRGQSPIH | |
| | | | | | PSQCQAVRMPGCWPQASKPLDPALGRGAPAGR | |
| | | | | | GHVFGKPEEPSTQRGPAQRESVGLVSMFRGLGI | |
| | | | | | ETVSKTPLKREMLPSGRGILGRGLSANLVRKDRE | |
| | | | | | ELSPTFWDPKVLAAGDSKMAETSVGWSRTLGRG | |
| | | | | | SSDASLLPLGRAAGGISREVDKPPCTFSTPSRGP | |
| | | | | | PQLSSPPALPQSPLHSPDRPLVLTVEHKEKELIVK | |
| | | | | | QGSKGTPQSLGLNLVKIQCHNEAVYQYHVTFSPN | |
| | | | | | VECKSMRFGMLKDHQAVTGNVTAFDGSILYLPVK | |
| | | | | | LQQVLELKSQRKTDSAEISIKIQMTKILEPCSDLCIP | |
| | | | | | FYNVVFRRVMKLLDMKLVGRNFYDPTSAMVLQQ | |
| | | | | | HRLQIWPGYAASIRRTDGGLFLLADVSHKVIRNDC | |
| | | | | | VLDVMHAIYQQNKEHFQDECTKLLVGNIVITRYNN | |
| | | | | | RTYRIDDVDWNKTPKDSFTMSDGKEITFLEYYSK | |
| | | | | | NYGITVKEEDQPLLIHRPSERQDNHGMLLKGEILL | |
| | | | | | LPELSFMTGIPEKMKKDFRAMKDLAQQINLSPKQ | |
| | | | | | HHSALECLLQRIAKNEAATNELMRWGLRLQKDVH | |
| | | | | | KIEGRVLPMERINLKNTSFITSQELNVWKEVTRDP | |
| | | | | | SILTIPMHFWALFYPKRAMDQARELVNMLEKIAGP | |
| | | | | | IGMRMSPPAWVELKDDRIETYVRTIQSTLGAEGKI | |
| | | | | | QMVVCIIMGPRDDLYGAIKKLCCVQSPVPSQVVN | |
| | | | | | VRTIGQPTRLRSVAQKILLQINCKLGGELWGVDIP | |
| | | | | | LKQLMVIGMDVYHDPSRGMRSVVGFVASINLTLT | |
| | | | | | KWYSRVVFQMPHQEIVDSLKLCLVGSLKKFYEVN | |
| | | | | | HCLPEKIVVYRDGVSDGQLKTVANYEIPQLQKCF | |
| | | | | | EAFENYQPKMVVFVVQKKISTNLYLAAPQNFVTP | |
| | | | | | TPGTVVDHTITSCEWVDFYLLAHHVRQGCGIPTH | |
| | | | | | YVCVLNTANLSPDHMQRLTFKLCHMYWNWPGTI | |
| | | | | | RVPAPCKYAHKLAFLSGHILHHEPAIQLCENLFFL | |
| | | | | | NPAFLYKVVKASS | |
| 228 | JHU1 | IPL | 0.2332162 | PIWIL2 | MDPFRPSFRGQSPIHPSQCQAVRMPGCWPQAS | Core Human ORF Only |
| | 9836 | TO | 1 | | KPLDPALGRGAPAGRGHVFGKPEEPSTQRGPAQ | |
| | | X | | | RESVGLVSMFRGLGIETVSKTPLKREMLPSGRGIL | |
| | | | | | GRGLSANLVRKDREELSPTFWDPKVLAAGDSKM | |
| | | | | | AETSVGWSRTLGRGSSDASLLPLGRAAGGISREV | |
| | | | | | DKPPCTFSTPSRGPPQLSSPPALPQSPLHSPDRP | |
| | | | | | LVLTVEHKEKELIVKQGSKGTPQSLGLNLVKIQCH | |
| | | | | | NEAVYQYHVTFSPNVECKSMRFGMLKDHQAVTG | |
| | | | | | NVTAFDGSILYLPVKLQQVLELKSQRKTDSAEISIK | |
| | | | | | IQMTKILEPCSDLCIPFYNVVFRRVMKLLDMKLVG | |
| | | | | | RNFYDPTSAMVLQQHRLQIWPGYAASIRRTDGGL | |
| | | | | | FLLADVSHKVIRNDCVLDVMHAIYQQNKEHFQDE | |
| | | | | | CTKLLVGNIVITRYNNRTYRIDDVDWNKTPKDSFT | |
| | | | | | MSDGKEITFLEYYSKNYGITVKEEDQPLLIHRPSE | |
| | | | | | RQDNHGMLLKGEILLLPELSFMTGIPEKMKKDFR | |
| | | | | | AMKDLAQQINLSPKQHHSALECLLQRIAKNEAAT | |
| | | | | | NELMRWGLRLQKDVHKIEGRVLPMERINLKNTSFI | |
| | | | | | TSQELNVWKEVTRDPSILTIPMHFWALFYPKRAM | |
| | | | | | DQARELVNMLEKIAGPIGMRMSPPAWVELKDDRI | |
| | | | | | ETYVRTIQSTLGAEGKIQMVVCIIMGPRDDLYGAIK | |
| | | | | | KLCCVQSPVPSQVVNVRTIGQPTRLRSVAQKILL | |
| | | | | | QINCKLGGELWGVDIPLKQLMVIGMDVYHDPSRG | |
| | | | | | MRSVVGFVASINLTLTKWYSRVVFQMPHQEIVDS | |
| | | | | | LKLCLVGSLKKFYEVNHCLPEKIVVYRDGVSDGQ | |
| | | | | | LKTVANYEIPQLQKCFEAFENYQPKMVVFVVQKKI | |
| | | | | | STNLYLAAPQNFVTPTPGTVVDHTITSCEWVDFY | |
| | | | | | LLAHHVRQGCGIPTHYVCVLNTANLSPDHMQRLT | |
| | | | | | FKLCHMYWNWPGTIRVPAPCKYAHKLAFLSGHIL | |
| | | | | | HHEPAIQLCENLFFL | |
| 229 | JHU1 | IPL | 0.3692623 | ZNF52 | MSPILGYWKIKGLVQPTRLLLEYLEEKYEEHLYER | Full Literal Sequence- |
| | 9892 | TO | 6 | 1 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSRRKQAKPRSLKD | |
| | | | | | PNCKLEDKTEDGEALDCKKRPEDGEELEDEAVH | |
| | | | | | SCDSCLQVFESLSDITEHKINQCQLTDGVDVEDD | |
| | | | | | PTCSWPASSPSSKDQTSPSHGEGCDFGEEEGG | |
| | | | | | PGLPYPCQFCDKSFSRLSYLKHHEQSHSDKLPFK | |
| | | | | | CTYCSRLFKHKRSRDRHIKLHTGDKKYHCSECDA | |
| | | | | | AFSRSDHLKIHLKTHTSNKPYKCAICRRGFLSSSS | |
| | | | | | LHGHMQVHERNKDGSQSGSRMEDWKMKDTQK | |
| | | | | | CSQCEEGFDFPEDLQKHIAECHPECSPNEDRAAL | |
| | | | | | QCVYCHELFVEETSLMNHMEQVHSGEKKNSCSI | |
| | | | | | CSESFHTVEELYSHMDSHQQPESCNHSNSPSLV | |
| | | | | | TVGYTSVSSTTPDSNLSVDSSTMVEAAPPIPKSR | |
| | | | | | GRKRAAQQTPDMTGPSSKQAKVTYSCIYCNKQL | |
| | | | | | FSSLAVLQIHLKTMHLDKPEQAHICQYCLEVLPSL | |
| | | | | | YNLNEHLKQVHEAQDPGLIVSAMPAIVYQCNFCS | |
| | | | | | EVVNDLNTLQEHIRCSHGFANPAAKDSNAFFCPH | |
| | | | | | CYMGFLTDSSLEEHIRQVHCDLSGSRFGSPVLGT | |
| | | | | | PKEPWEVYSCSYCTNSPIFNSVLKLNKHIKENHK | |
| | | | | | NIPLALNYIHNGKKSRALSPLSPVAIEQTSLKMMQ | |
| | | | | | AVGGAPARPTGEYICNQCGAKYTSLDSFQTHLKT | |
| | | | | | HLDTVLPKLTCPQCNKEFPNQESLLKHVTIHFMIT | |
| | | | | | STYYICESCDKQFTSVDDLQKHLLDMHTFVFFRC | |
| | | | | | TLCQEVFDSKVSIQLHLAVKHSNEKKVYRCTSCN | |
| | | | | | WDFRNETDLQLHVKHNHLENQGKVHKCIFCGES | |
| | | | | | FGTEVELQCHITTHSKKYNCKFCSKAFHAIILLEKH | |
| | | | | | LREKHCVFETKTPNCGTNGASEQVQKEEVELQTL | |
| | | | | | LTNSQESHNSHDGSEEDVDTSEPMYGCDICGAA | |
| | | | | | YTMETLLQNHQLRDHNIRPGESAIVKKKAELIKGN | |
| | | | | | YKCNVCSRTFFSENGLREHMQTHLGPVKHYMCP | |
| | | | | | ICGERFPSLLTLTEHKVTHSKSLDTGNCRICKMPL | |
| | | | | | QSEEEFLEHCQMHPDLRNSLTGFRCVVCMQTVT | |
| | | | | | STLELKIHGTFHMQKTGNGSAVQTTGRGQHVQK | |
| | | | | | LYKCASCLKEFRSKQDLVKLDINGLPYGLCAGCV | |
| | | | | | NLSKSASPGINVPPGTNRPGLGQNENLSAIEGKG | |
| | | | | | KVGGLKTRCSSCNVKFESESELQNHIQTIHRELVP | |
| | | | | | DSNSTQLKTPQVSPMPRISPSQSDEKKTYQCIKC | |
| | | | | | QMVFYNEWDIQVHVANHMIDEGLNHECKLCSQT | |
| | | | | | FDSPAKLQCHLIEHSFEGMGGTFKCPVCFTVFVQ | |
| | | | | | ANKLQQHIFSAHGQEDKIYDCTQCPQKFFFQTEL | |
| | | | | | QNHTMTQHSSNPAFLYKVVKASS | |
| 230 | JHU1 | IPI_ | 0.3692623 | ZNF52 | MSRRKQAKPRSLKDPNCKLEDKTEDGEALDCKK | Core Human ORF Only |
| | 9892 | TO | 6 | 1 | RPEDGEELEDEAVHSCDSCLQVFESLSDITEHKIN | |
| | | X | | | QCQLTDGVDVEDDPTCSWPASSPSSKDQTSPSH | |
| | | | | | GEGCDFGEEEGGPGLPYPCQFCDKSFSRLSYLK | |
| | | | | | HHEQSHSDKLPFKCTYCSRLFKHKRSRDRHIKLH | |
| | | | | | TGDKKYHCSECDAAFSRSDHLKIHLKTHTSNKPY | |
| | | | | | KCAICRRGFLSSSSLHGHMQVHERNKDGSQSGS | |
| | | | | | RMEDWKMKDTQKCSQCEEGFDFPEDLQKHIAEC | |
| | | | | | HPECSPNEDRAALQCVYCHELFVEETSLMNHME | |
| | | | | | QVHSGEKKNSCSICSESFHTVEELYSHMDSHQQ | |
| | | | | | PESCNHSNSPSLVTVGYTSVSSTTPDSNLSVDSS | |
| | | | | | TMVEAAPPIPKSRGRKRAAQQTPDMTGPSSKQA | |
| | | | | | KVTYSCIYCNKQLFSSLAVLQIHLKTMHLDKPEQA | |
| | | | | | HICQYCLEVLPSLYNLNEHLKQVHEAQDPGLIVSA | |
| | | | | | MPAIVYQCNFCSEVVNDLNTLQEHIRCSHGFANP | |
| | | | | | AAKDSNAFFCPHCYMGFLTDSSLEEHIRQVHCDL | |
| | | | | | SGSRFGSPVLGTPKEPVVEVYSCSYCTNSPIFNS | |
| | | | | | VLKLNKHIKENHKNIPLALNYIHNGKKSRALSPLSP | |
| | | | | | VAIEQTSLKMMQAVGGAPARPTGEYICNQCGAK | |
| | | | | | YTSLDSFQTHLKTHLDTVLPKLTCPQCNKEFPNQ | |
| | | | | | ESLLKHVTIHFMITSTYYICESCDKQFTSVDDLQK | |
| | | | | | HLLDMHTFVFFRCTLCQEVFDSKVSIQLHLAVKHS | |
| | | | | | NEKKVYRCTSCNWDFRNETDLQLHVKHNHLENQ | |
| | | | | | GKVHKCIFCGESFGTEVELQCHITTHSKKYNCKF | |
| | | | | | CSKAFHAIILLEKHLREKHCVFETKTPNCGTNGAS | |
| | | | | | EQVQKEEVELQTLLTNSQESHNSHDGSEEDVDT | |
| | | | | | SEPMYGCDICGAAYTMETLLQNHQLRDHNIRPGE | |
| | | | | | SAIVKKKAELIKGNYKCNVCSRTFFSENGLREHM | |
| | | | | | QTHLGPVKHYMCPICGERFPSLLTLTEHKVTHSK | |
| | | | | | SLDTGNCRICKMPLQSEEEFLEHCQMHPDLRNSL | |
| | | | | | TGFRCVVCMQTVTSTLELKIHGTFHMQKTGNGSA | |
| | | | | | VQTTGRGQHVQKLYKCASCLKEFRSKQDLVKLDI | |
| | | | | | NGLPYGLCAGCVNLSKSASPGINVPPGTNRPGLG | |
| | | | | | QNENLSAIEGKGKVGGLKTRCSSCNVKFESESEL | |
| | | | | | QNHIQTIHRELVPDSNSTQLKTPQVSPMPRISPSQ | |
| | | | | | SDEKKTYQCIKCQMVFYNEWDIQVHVANHMIDEG | |
| | | | | | LNHECKLCSQTFDSPAKLQCHLIEHSFEGMGGTF | |
| | | | | | KCPVCFTVFVQANKLQQHIFSAHGQEDKIYDCTQ | |
| | | | | | CPQKFFFQTELQNHTMTQHSS | |
| 231 | JHU2 | IPI_ | 0.28472181 | ANP32 | MSPILGYWKIKGLVQPTRLLLEYLEEKYEEHLYER | Full Literal Sequence- |
| | 5691 | TO | 2 | C | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEMGRRIHSELRNR | |
| | | | | | APSDVKELALDNSRSNEGKLEALTDEFEELEFLSK | |
| | | | | | INGGLTSISDLPKLKLRKLELRVSGGLEVLAEKCP | |
| | | | | | NLTHLYLSGNKIKDLSTIEPLKQLENLKSLDLFNCE | |
| | | | | | VTNLNDYGENVFKLLLQLTYLDSCYWDHKEAPYS | |
| | | | | | DIEDHVEGLDDEEEGEHEEEYDEDAQVVEDEEG | |
| | | | | | EEEEEEGEEEDVSGGDEEDEEGYNDGEVDGEE | |
| | | | | | DEEELGEEERGQKRKNPAFLYKVVKASS | |
| 232 | JHU2 | IPL | 0.28472181 | ANP32 | MEMGRRIHSELRNRAPSDVKELALDNSRSNEGK | Core Human ORF Only |
| | 5691 | TO | 2 | C | LEALTDEFEELEFLSKINGGLTSISDLPKLKLRKLE | |
| | | X | | | LRVSGGLEVLAEKCPNLTHLYLSGNKIKDLSTIEPL | |
| | | | | | KQLENLKSLDLFNCEVTNLNDYGENVFKLLLQLTY | |
| | | | | | LDSCYWDHKEAPYSDIEDHVEGLDDEEEGEHEE | |
| | | | | | EYDEDAQVVEDEEGEEEEEEGEEEDVSGGDEED | |
| | | | | | EEGYNDGEVDGEEDEEELGEEERGQKRK | |
| 233 | JHU2 | IPL | 0.57241945 | AQP11 | MSPILGYWKIKGLVQPTRLLLEYLEEKYEEHLYER | Full Literal Sequence- |
| | 7612 | TO | 6 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSPLLGLRSELQDTC | |
| | | | | | TSLGLMLSVVLLMGLARVVARQQLHRPVAHAFVL | |
| | | | | | EFLATFQLCCCTHELQLLSEQHPAHPTWTLTLVY | |
| | | | | | FFSLVHGLTLVGTSSNPCGVMMQMMLGGMSPET | |
| | | | | | GAVRLLAQLVSALCSRYCTSALWSLGLTQYHVSE | |
| | | | | | RSFACKNPIRVDLLKAVITEAVCSFLFHSALLHFQE | |
| | | | | | VRTKLRIHLLAALITFLVYAGGSLTGAVFNPALALS | |
| | | | | | LHFMCFDEAFPQFFIVYWLAPSLGILLMILMFSFFL | |
| | | | | | PWLHNNHTINKKENPAFLYKVVKASS | |
| 234 | JHU2 | IPL | 0.57241945 | AQP11 | MSPLLGLRSELQDTCTSLGLMLSVVLLMGLARVV | Core Human ORF Only |
| | 7612 | TO | 6 | | ARQQLHRPVAHAFVLEFLATFQLCCCTHELQLLS | |
| | | X | | | EQHPAHPTWTLTLVYFFSLVHGLTLVGTSSNPCG | |
| | | | | | VMMQMMLGGMSPETGAVRLLAQLVSALCSRYCT | |
| | | | | | SALWSLGLTQYHVSERSFACKNPIRVDLLKAVITE | |
| | | | | | AVCSFLFHSALLHFQEVRTKLRIHLLAALITFLVYA | |
| | | | | | GGSLTGAVFNPALALSLHFMCFDEAFPQFFIVYW | |
| | | | | | LAPSLGILLMILMFSFFLPWLHNNHTINKKE | |
| 235 | JHU2 | IPL | 0.1051835 | MRPL3 | MSPILGYWKIKGLVQPTRLLLEYLEEKYEEHLYER | Full Literal Sequence- |
| | 9116 | TO | 3 | 9 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEALAMGSRALRLW | |
| | | | | | LVAPGGGIKWRFIATSSASQLSPTELTEMRNDLF | |
| | | | | | NKEKARQLSLTPRTEKIEVKHVGKTDPGTVFVMN | |
| | | | | | KNISTPYSCAMHLSEWYCRKSILALVDGQPWDM | |
| | | | | | YKPLTKSCEIKFLTFKDCDPGEVNKAYWRSCAMM | |
| | | | | | MGCVIERAFKDEYMVNLVRAPEVPVISGAFCYDV | |
| | | | | | VLDSKLDEWMPTKENLRSFTKDAHALIYKDLPFET | |
| | | | | | LEVEAKVALEIFQHSKYKVDFIEEKASQNPERIVKL | |
| | | | | | HRIGDFIDVSEGPLIPRTSICFQYEVSAVHNLQPT | |
| | | | | | QPSLIRRFQGVSLPVHLRAHFTIWDKLLERSRKM | |
| | | | | | TPFPILLLFTTQSFFTTSPESYLLHGTVSE | |
| 236 | JHU2 | IPL | 0.1051835 | MRPL3 | MEALAMGSRALRLWLVAPGGGIKWRFIATSSASQ | Core Human ORF Only |
| | 9116 | TO | 3 | 9 | LSPTELTEMRNDLFNKEKARQLSLTPRTEKIEVKH | |
| | | X | | | VGKTDPGTVFVMNKNISTPYSCAMHLSEWYCRK | |
| | | | | | SILALVDGQPWDMYKPLTKSCEIKFLTFKDCDPG | |
| | | | | | EVNKAYWRSCAMMMGCVIERAFKDEYMVNLVRA | |
| | | | | | PEVPVISGAFCYDVVLDSKLDEWMPTKENLRSFT | |
| | | | | | KDAHALIYKDLPFETLEVEAKVALEIFQHSKYKVDF | |
| | | | | | IEEKASQNPERIVKLHRIGDFIDVSEGPLIPRTSICF | |
| | | | | | QYEVSAVHNLQPTQPSLIRRFQGVSLPVHLRAHF | |
| | | | | | TIWDKLLERSRKMTPFPILLLFTTQSFFTTSPESYL | |
| | | | | | LHGTVSE | |
| 237 | JHU2 | IPI_ | 0.2732131 | NR2F6 | MSPILGYWKIKGLVQPTRLLLEYLEEKYEEHLYER | Full Literal Sequence- |
| | 9217 | TO | 2 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAMVTGGWGGPGG | |
| | | | | | DTNGVDKAGGYPRAAEDDSASPPGAASDAEPGD | |
| | | | | | EERPGLQVDCVVCGDKSSGKHYGVFTCEGCKSF | |
| | | | | | FKRSIRRNLSYTCRSNRDCQIDQHHRNQCQYCR | |
| | | | | | LKKCFRVGMRKEAVQRGRIPHSLPGAVAASSGS | |
| | | | | | PPGSALAAVASGGDLFPGQPVSELIAQLLRAEPY | |
| | | | | | PAAAGRFGAGGGAAGAVLGIDNVCELAARLLFST | |
| | | | | | VEWARHAPFFPELPVADQVALLRLSWSELFVLNA | |
| | | | | | AQAALPLHTAPLLAAAGLHAAPMAAERAVAFMDQ | |
| | | | | | VRAFQEQVDKLGRLQVDSAEYGCLKAIALFTPDA | |
| | | | | | CGLSDPAHVESLQEKAQVALTEYVRAQYPSQPQ | |
| | | | | | RFGRLLLRLPALRAVPASLISQLFFMRLVGKTPIET | |
| | | | | | LIRDMLLSGSTFNWPYGSGQNPAFLYKVVKASS | |
| 238 | JHU2 | IPL | 0.2732131 | NR2F6 | MAMVTGGWGGPGGDTNGVDKAGGYPRAAEDD | Core Human ORF Only |
| | 9217 | TO | 2 | | SASPPGAASDAEPGDEERPGLQVDCVVCGDKSS | |
| | | X | | | GKHYGVFTCEGCKSFFKRSIRRNLSYTCRSNRDC | |
| | | | | | QIDQHHRNQCQYCRLKKCFRVGMRKEAVQRGRI | |
| | | | | | PHSLPGAVAASSGSPPGSALAAVASGGDLFPGQ | |
| | | | | | PVSELIAQLLRAEPYPAAAGRFGAGGGAAGAVLG | |
| | | | | | IDNVCELAARLLFSTVEWARHAPFFPELPVADQV | |
| | | | | | ALLRLSWSELFVLNAAQAALPLHTAPLLAAAGLHA | |
| | | | | | APMAAERAVAFMDQVRAFQEQVDKLGRLQVDSA | |
| | | | | | EYGCLKAIALFTPDACGLSDPAHVESLQEKAQVA | |
| | | | | | LTEYVRAQYPSQPQRFGRLLLRLPALRAVPASLIS | |
| | | | | | QLFFMRLVGKTPIETLIRDMLLSGSTFNWPYGSG | |
| | | | | | Q | |
| 239 | JHU2 | IPL | 0.44530695 | ATP6V | MSPILGYWKIKGLVQPTRLLLEYLEEKYEEHLYER | Full Literal Sequence- |
| | 9445 | TO | 5 | 0A4 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVSVFRSEEMCLSQ | |
| | | | | | LFLQVEAAYCCVAELGELGLVQFKDLNMNVNSFQ | |
| | | | | | RKFVNEVRRCESLERILRFLEDEMQNEIVVQLLEK | |
| | | | | | SPLTPLPREMITLETVLEKLEGELQEANQNQQALK | |
| | | | | | QSFLELTELKYLLKKTQDFFETETNLADDFFTEDT | |
| | | | | | SGLLELKAVPAYMTGKLGFIAGVINRERMASFERL | |
| | | | | | LWRICRGNVYLKFSEMDAPLEDPVTKEEIQKNIFII | |
| | | | | | FYQGEQLRQKIKKICDGFRATVYPCPERAVERRE | |
| | | | | | MLESVNVRLEDLITVITQTESHRQRLLQEAAANW | |
| | | | | | HSWLIKVQKMKAVYHILNMCNIDVTQQCVIAEIWF | |
| | | | | | PVADATRIKRALEQGMELSGSSMAPIMTTVQSKT | |
| | | | | | APPTFNRTNKFTAGFQNIVDAYGVGSYREINPAP | |
| | | | | | YTIITFPFLFAVMFGDCGHGTVMLLAALWMILNER | |
| | | | | | RLLSQKTDNEIWNTFFHGRYLILLMGIFSIYTGLIY | |
| | | | | | NDCFSKSLNIFGSSWSVQPMFRNGTWNTHVMEE | |
| | | | | | SLYLQLDPAIPGVYFGNPYPFGIDPIWNLASNKLT | |
| | | | | | FLNSYKMKMSVILGIVQMVFGVILSLFNHIYFRRTL | |
| | | | | | NIILQFIPEMIFILCLFGYLVFMIIFKWCCFDVHVSQ | |
| | | | | | HAPSILIHFINMFLFNYSDSSNAPLYKHQQEVQSF | |
| | | | | | FVVMALISVPWMLLIKPFILRASHRKSQLQASRIQE | |
| | | | | | DATENIEGDSSSPSSRSGQRTSADTHGALDDHG | |
| | | | | | EEFNFGDVFVHQAIHTIEYCLGCISNTASYLRLWA | |
| | | | | | LSLAHAQLSEVLWTMVMNSGLQTRGWGGIVGVF | |
| | | | | | IIFAVFAVLTVAILLIMEGLSAFLHALRLHVWEFQNK | |
| | | | | | FYVGDGYKFSPFSFKHILDGTAEE | |
| 240 | JHU2 | IPL | 0.44530695 | ATP6V | MVSVFRSEEMCLSQLFLQVEAAYCCVAELGELGL | Core Human ORF Only |
| | 9445 | TO | 5 | 0A4 | VQFKDLNMNVNSFQRKFVNEVRRCESLERILRFL | |
| | | X | | | EDEMQNEIVVQLLEKSPLTPLPREMITLETVLEKL | |
| | | | | | EGELQEANQNQQALKQSFLELTELKYLLKKTQDF | |
| | | | | | FETETNLADDFFTEDTSGLLELKAVPAYMTGKLG | |
| | | | | | FIAGVINRERMASFERLLWRICRGNVYLKFSEMD | |
| | | | | | APLEDPVTKEEIQKNIFIIFYQGEQLRQKIKKICDGF | |
| | | | | | RATVYPCPERAVERREMLESVNVRLEDLITVITQT | |
| | | | | | ESHRQRLLQEAAANWHSWLIKVQKMKAVYHILN | |
| | | | | | MCNIDVTQQCVIAEIWFPVADATRIKRALEQGMEL | |
| | | | | | SGSSMAPIMTTVQSKTAPPTFNRTNKFTAGFQNI | |
| | | | | | VDAYGVGSYREINPAPYTIITFPFLFAVMFGDCGH | |
| | | | | | GTVMLLAALWMILNERRLLSQKTDNEIWNTFFHG | |
| | | | | | RYLILLMGIFSIYTGLIYNDCFSKSLNIFGSSWSVQ | |
| | | | | | PMFRNGTWNTHVMEESLYLQLDPAIPGVYFGNP | |
| | | | | | YPFGIDPIWNLASNKLTFLNSYKMKMSVILGIVQMV | |
| | | | | | FGVILSLFNHIYFRRTLNIILQFIPEMIFILCLFGYL | |
| | | | | | VFMIIFKWCCFDVHVSQHAPSILIHFINMFLFNYSDS | |
| | | | | | SNAPLYKHQQEVQSFFVVMALISVPWMLLIKPFIL | |
| | | | | | RASHRKSQLQASRIQEDATENIEGDSSSPSSRSG | |
| | | | | | QRTSADTHGALDDHGEEFNFGDVFVHQAIHTIEY | |
| | | | | | CLGCISNTASYLRLWALSLAHAQLSEVLWTMVMN | |
| | | | | | SGLQTRGWGGIVGVFIIFAVFAVLTVAILLIMEGLS | |
| | | | | | AFLHALRLHWVEFQNKFYVGDGYKFSPFSFKHIL | |
| | | | | | DGTAEE | |
| 241 | JHU2 | IPI_ | 0.90289091 | KCND2 | MSPILGYWKIKGLVQPTRLLLEYLEEKYEEHLYER | Full Literal Sequence- |
| | 9882 | TO | 6 | | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAGVAAWLPFARA | |
| | | | | | AAIGWMPVASGPMPAPPRQERKRTQDALIVLNVS | |
| | | | | | GTRFQTWQDTLERYPDTLLGSSERDFFYHPETQ | |
| | | | | | QYFFDRDPDIFRHILNFYRTGKLHYPRHECISAYD | |
| | | | | | EELAFFGLIPEIIGDCCYEEYKDRRRENAERLQDD | |
| | | | | | ADTDTAGESALPTMTARQRVWRAFENPHTSTMA | |
| | | | | | LVFYYVTGFFIAVSVIANVVETVPCGSSPGHIKELP | |
| | | | | | CGERYAVAFFCLDTACVMIFTVEYLLRLAAAPSRY | |
| | | | | | RFVRSVMSIIDVVAILPYYIGLVMTDNEDVSGAFVT | |
| | | | | | LRVFRVFRIFKFSRHSQGLRILGYTLKSCASELGF | |
| | | | | | LLFSLTMAIIIFATVMFYAEKGSSASKFTSIPAAFW | |
| | | | | | YTIVTMTTLGYGDMVPKTIAGKIFGSICSLSGVLVI | |
| | | | | | ALPVPVIVSNFSRIYHQNQRADKRRAQKKARLARI | |
| | | | | | RAAKSGSANAYMQSKRNGLLSNQLQSSEDEQAF | |
| | | | | | VSKSGSSFETQHHHLLHCLEKTTNHEFVDEQVFE | |
| | | | | | ESCMEVATVNRPSSHSPSLSSQQGVTSTCCSRR | |
| | | | | | HKKTFRIPNANVSGSHQGSIQELSTIQIRCVERTP | |
| | | | | | LSNSRSSLNAKMEECVKLNCEQPYVTTAIISIPTPP | |
| | | | | | VTTPEGDDRPESPEYSGGNIVRVSAL | |
| 242 | JHU2 | IPL | 0.90289091 | KCND2 | MAAGVAAWLPFARAAAIGWMPVASGPMPAPPR | Core Human ORF Only |
| | 9882 | TO | 6 | | QERKRTQDALIVLNVSGTRFQTWQDTLERYPDTL | |
| | | X | | | LGSSERDFFYHPETQQYFFDRDPDIFRHILNFYRT | |
| | | | | | GKLHYPRHECISAYDEELAFFGLIPEIIGDCCYEEY | |
| | | | | | KDRRRENAERLQDDADTDTAGESALPTMTARQR | |
| | | | | | VWRAFENPHTSTMALVFYYVTGFFIAVSVIANVVE | |
| | | | | | TVPCGSSPGHIKELPCGERYAVAFFCLDTACVMIF | |
| | | | | | TVEYLLRLAAAPSRYRFVRSVMSIIDVVAILPYYIG | |
| | | | | | LVMTDNEDVSGAFVTLRVFRVFRIFKFSRHSQGL | |
| | | | | | RILGYTLKSCASELGFLLFSLTMAIIIFATVMFYAEK | |
| | | | | | GSSASKFTSIPAAFWYTIVTMTTLGYGDMVPKTIA | |
| | | | | | GKIFGSICSLSGVLVIALPVPVIVSNFSRIYHQNQR | |
| | | | | | ADKRRAQKKARLARIRAAKSGSANAYMQSKRNG | |
| | | | | | LLSNQLQSSEDEQAFVSKSGSSFETQHHHLLHCL | |
| | | | | | EKTTNHEFVDEQVFEESCMEVATVNRPSSHSPSL | |
| | | | | | SSQQGVTSTCCSRRHKKTFRIPNANVSGSHQGSI | |
| | | | | | QELSTIQIRCVERTPLSNSRSSLNAKMEECVKLNC | |
| | | | | | EQPYVTTAIISIPTPPVTTPEGDDRPESPEYSGGNI | |
| | | | | | VRVSAL | |
| 243 | JHU3 | IPL | 0.53535227 | OR11H | MSPILGYWKIKGLVQPTRLLLEYLEEKYEEHLYER | Full Literal Sequence- |
| | 0083 | TO | 6 | 12 | DEGDKWRNKKFELGLEFPNLPYYIDGDVKLTQSM | (including GST-tag, 6- |
| | | X | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMCPLTLQVTGLMNV | |
| | | | | | SEPNSSFAFVNEFILQGFTCEWTIQIFLFSLFTTTY | |
| | | | | | ALTITGNGAIAFVLWCDWRLHTPMYMFLGNFSFL | |
| | | | | | EIWYVSSTVPKMLVNFLSEKKNISFAGCFLQFYFF | |
| | | | | | FSLGTSECLLLTVMAFDQYLAICRPLLYPNIMTGH | |
| | | | | | LCAKLVILCWVCGFLWFLIPIVLISQMPFCGPNIIDH | |
| | | | | | VVCDPGPRFALDCVSAPRIQLFCYTLSSLVIFGNF | |
| | | | | | LFIIGSYTLVLKAVLGMPSSTGRHKAFSTCGSHLA | |
| | | | | | VVSLCYSSLMVMYVSPGLGHSTGMQKIETLFYAM | |
| | | | | | VTPLFNPLIYSLQNKEIKAALRKVLGSSNII | |
| 244 | JHU3 | IPI_ | 0.53535227 | OR11H | MCPLTLQVTGLMNVSEPNSSFAFVNEFILQGFTC | Core Human ORF Only |
| | 0083 | TO | 6 | 12 | EWTIQIFLFSLFTTTYALTITGNGAIAFVLWCDWRL | |
| | | X | | | HTPMYMFLGNFSFLEIWYVSSTVPKMLVNFLSEK | |
| | | | | | KNISFAGCFLQFYFFFSLGTSECLLLTVMAFDQYL | |
| | | | | | AICRPLLYPNIMTGHLCAKLVILCVWCGFLWFLIPI | |
| | | | | | VLISQMPFCGPNIIDHVVCDPGPRFALDCVSAPRI | |
| | | | | | QLFCYTLSSLVIFGNFLFIIGSYTLVLKAVLGMPSS | |
| | | | | | TGRHKAFSTCGSHLAVVSLCYSSLMVMYVSPGL | |
| | | | | | GHSTGMQKIETLFYAMVTPLFNPLIYSLQNKEIKA | |
| | | | | | ALRKVLGSSNII | |
| 245 | JHUO | NIV | 0.55802062 | IGIP | MSPILGYWKIKGLVQPTRLLLEYLEEKYEEHLYER | Full Literal Sequence- |
| | 1492 | O_ | 2 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKKRSVSGCNITIFA | |
| | | | | | VMFSHLSAGKSPCGNQANVLCISRLEFVQYQS | |
| 246 | JHUO | NIV | 0.55802062 | IGIP | MKKRSVSGCNITIFAVMFSHLSAGKSPCGNQANV | Core Human ORF Only |
| | 1492 | O_ | 2 | | LCISRLEFVQYQS | |
| | | RFS | | | | |
| 247 | JHUO | NIV | 1.12240014 | EMC2 | MSPILGYWKIKGLVQPTRLLLEYLEEKYEEHLYER | Full Literal Sequence- |
| | 1818 | O_ | 9 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAKVSELYDVTWEE | |
| | | | | | MRDKMRKWREENSRNSEQIVEVGEELINEYASKL | |
| | | | | | GDDIWIIYEQVMIAALDYGRDDLALFCLQELRRQF | |
| | | | | | PGSHRVKRLTGMRFEAMERYDDAIQLYDRILQED | |
| | | | | | PTNTAARKRKIAIRKAQGKNVEAIRELSEYLEQFV | |
| | | | | | GDQEAWHELAELYINEHDYAKAAFCLEELMMTNP | |
| | | | | | HNHLYCQQYAEVKYTQGGLENLELSRKYFAQAL | |
| | | | | | KLNNRNMRALFGLYMSASHIASNPKASAKTKKDN | |
| | | | | | MKYASWAASQINRAYQFAGRSKKETKYSLKAVE | |
| | | | | | DMLETLQITQS | |
| 248 | JHUO | NIV | 1.12240014 | EMC2 | MAKVSELYDVTWEEMRDKMRKWREENSRNSEQ | Core Human ORF Only |
| | 1818 | O_ | 9 | | IVEVGEELINEYASKLGDDIWIIYEQVMIAALDYGR | |
| | | RFS | | | DDLALFCLQELRRQFPGSHRVKRLTGMRFEAME | |
| | | | | | RYDDAIQLYDRILQEDPTNTAARKRKIAIRKAQGK | |
| | | | | | NVEAIRELSEYLEQFVGDQEAWHELAELYINEHD | |
| | | | | | YAKAAFCLEELMMTNPHNHLYCQQYAEVKYTQG | |
| | | | | | GLENLELSRKYFAQALKLNNRNMRALFGLYMSAS | |
| | | | | | HIASNPKASAKTKKDNMKYASWAASQINRAYQFA | |
| | | | | | GRSKKETKYSLKAVEDMLETLQITQS | |
| 249 | JHUO | NIV | 0.74850387 | C2orf15 | MSPILGYWKIKGLVQPTRLLLEYLEEKYEEHLYER | Full Literal Sequence- |
| | 3764 | O_ | 5 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSSTLGKLSNQVEET | |
| | | | | | LPLLKKVPANYFHICSAILMGFSLSKSATQVSAIHM | |
| | | | | | DSKVDDHLIRGTEKSRLEPATQLFQNTKKIRLEDT | |
| | | | | | NQENFTRIEGTGTGSLSGKALGSVVYVKESDGLE | |
| | | | | | MTDVE | |
| 250 | JHUO | NIV | 0.74850387 | C2orf15 | MSSTLGKLSNQVEETLPLLKKVPANYFHICSAILM | Core Human ORF Only |
| | 3764 | O_ | 5 | | GFSLSKSATQVSAIHMDSKVDDHLIRGTEKSRLEP | |
| | | RFS | | | ATQLFQNTKKIRLEDTNQENFTRIEGTGTGSLSGK | |
| | | | | | ALGSVVYVKESDGLEMTDVE | |
| 251 | JHUO | NIV | 0.81827563 | CLPS | MSPILGYWKIKGLVQPTRLLLEYLEEKYEEHLYER | Full Literal Sequence- |
| | 3776 | O_ | 1 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEKILILLLVALSVAY | |
| | | | | | AAPGPRGIIINLENGELCMNSAQCKSNCCQHSSA | |
| | | | | | LGLARCTSMASENSECSVKTLYGIYYKCPCERGL | |
| | | | | | TCEGDKTIVGSITNTNFGICHDAGRSKQ | |
| 252 | JHUO | NIV | 0.81827563 | CLPS | MEKILILLLVALSVAYAAPGPRGIIINLENGELCMN | Core Human ORF Only |
| | 3776 | O_ | 1 | | SAQCKSNCCQHSSALGLARCTSMASENSECSVKT | |
| | | RFS | | | LYGIYYKCPCERGLTCEGDKTIVGSITNTNFGICH | |
| | | | | | DAGRSKQ | |
| 253 | JHUO | NIV | 0.93836124 | CCDC1 | MSPILGYWKIKGLVQPTRLLLEYLEEKYEEHLYER | Full Literal Sequence- |
| | 4244 | O_ | 5 | 07 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGAVSLLGVVGLLL | |
| | | | | | VSALSGVLGDRANPDLRAHPGNAAHPGSGATEP | |
| | | | | | RRRPPLKDQRERTRAGSLPLGALYTAAVAAFVLY | |
| | | | | | KCLQGKDETAVLHEEASKQQPLQSEQQLAQLTQ | |
| | | | | | QLAQTEQHLNNLMAQLDPLFERVTTLAGAQQELL | |
| | | | | | NMKLWTIHELLQDSKPDKDMEASEPGEGSGGES | |
| | | | | | AGGGDKVSETGTFLISPHTEASRPLPEDFCLKED | |
| | | | | | EEEVGDSQAWEEPTNWSTETWNLATSWEVGRG | |
| | | | | | LRRRCSQAVAKGPSHSLGWEGGTTAEGRLKQSL | |
| | | | | | FS | |
| 254 | JHUO | NIV | 0.93836124 | CCDC1 | MAGAVSLLGVVGLLLVSALSGVLGDRANPDLRAH | Core Human ORF Only |
| | 4244 | O_ | 5 | 07 | PGNAAHPGSGATEPRRRPPLKDQRERTRAGSLP | |
| | | RFS | | | LGALYTAAVAAFVLYKCLQGKDETAVLHEEASKQ | |
| | | | | | QPLQSEQQLAQLTQQLAQTEQHLNNLMAQLDPL | |
| | | | | | FERVTTLAGAQQELLNMKLWTIHELLQDSKPDKD | |
| | | | | | MEASEPGEGSGGESAGGGDKVSETGTFLISPHT | |
| | | | | | EASRPLPEDFCLKEDEEEVGDSQAWEEPTNWST | |
| | | | | | ETWNLATSWEVGRGLRRRCSQAVAKGPSHSLG | |
| | | | | | WEGGTTAEGRLKQSLFS | |
| 255 | JHUO | NIV | 2.89926293 | COX16 | MSPILGYWKIKGLVQPTRLLLEYLEEKYEEHLYER | Full Literal Sequence- |
| | 4522 | O_ | 3 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMFAPAVMRAFRKNK | |
| | | | | | TLGYGVPMLLLIVGGSFGLREFSQIRYDAVKSKM | |
| | | | | | DPELEKKLKENKISLESEYEKIKDSKFDDWKNIRG | |
| | | | | | PRPWEDPDLLQGRNPESLKTKTT | |
| 256 | JHUO | NIV | 2.89926293 | COX16 | MFAPAVMRAFRKNKTLGYGVPMLLLIVGGSFGLR | Core Human ORF Only |
| | 4522 | O_ | 3 | | EFSQIRYDAVKSKMDPELEKKLKENKISLESEYEKI | |
| | | RFS | | | KDSKFDDWKNIRGPRPWEDPDLLQGRNPESLKT | |
| | | | | | KTT | |
| 257 | JHUO | NIV | 1.35682381 | TWF1 | MSPILGYWKIKGLVQPTRLLLEYLEEKYEEHLYER | Full Literal Sequence- |
| | 4886 | O_ | 2 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLYAATRATLKKEFG | |
| | | | | | GGHIKDEVFGTVKEDVSLHGYKKYLLSQSSPAPL | |
| | | | | | TAAEEELRQIKINEVQTDVGVDTKHQTLQGVAFPIS | |
| | | | | | REAFQALEKLNNRQLNYVQLEIDIKNEIIILANTTN | |
| | | | | | TELKDLPKRIPKDSARYHFFLYKHSHEGDYLESIV | |
| | | | | | FIYSMPGYTCSIRERMLYSSCKSRLLEIVERQLQM | |
| | | | | | DVIRKIEIDNGDELTADFLYEEVHPKQHAHKQSFA | |
| | | | | | KPKGPAGKRGIRRLIRGPAETEATTD | |
| 258 | JHUO | NIV | 1.35682381 | TWF1 | MLYAATRATLKKEFGGGHIKDEVFGTVKEDVSLH | Core Human ORF Only |
| | 4886 | O_ | 2 | | GYKKYLLSQSSPAPLTAAEEELRQIKINEVQTDVG | |
| | | RFS | | | VDTKHQTLQGVAFPISREAFQALEKLNNRQLNYVQ | |
| | | | | | LEIDIKNEIIILANTTNTELKDLPKRIPKDSARYHF | |
| | | | | | FLYKHSHEGDYLESIVFIYSMPGYTCSIRERMLYSS | |
| | | | | | CKSRLLEIVERQLQMDVIRKIEIDNGDELTADFLYE | |
| | | | | | EVHPKQHAHKQSFAKPKGPAGKRGIRRLIRGPAE | |
| | | | | | TEATTD | |
| 259 | JHUO | NIV | 1.30072023 | SLCO2 | MSPILGYWKIKGLVQPTRLLLEYLEEKYEEHLYER | Full Literal Sequence- |
| | 6988 | O_ | 6 | A1 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | |
| | | | | | PKLVCFKKRIEAIPQIDKYLKSSKYIAWPLQGWQA | ORF, occasional ‘post |
| | | | | | TFGGGDHPPKSDLVPRGSPGISGGGGGILGRGS | stop’ end sequence) |
| | | | | | HHHHHHGGGITSLYKKAGTMGLLPKLGASQGSD | |
| | | | | | TSTSRAGRCARSVFGNIKVFVLCQGLLQLCQLLYSA | |
| | | | | | YFKSSLTTIEKRFGLSSSSSGLISSLNEISNAILII | |
| | | | | | FVSYFGSRVHRPRLIGIGGLFLAAGAFILTLPHFLS | |
| | | | | | EPYQYTLASTGNNSRLQAELCQKHWQDLPPSKC | |
| | | | | | HSTTQNPQKETSSMWGLMVVAQLLAGIGTVPIQP | |
| | | | | | FGISYVDDFSEPSNSPLYISILFAISVFGPAFGYLL | |
| | | | | | GSVMLQIFVDYGRVNTAAVNLVPGDPRWIGAVWV | |
| | | | | | LGLLISSALLVLTSFPFFFFPRAMPIGAKRAPATAD | |
| | | | | | EARKLEEAKSRGSLVDFIKRFPCIFLRLLMNSLFVL | |
| | | | | | VVLAQCTFSSVIAGLSTFLNKFLEKQYGTSAAYAN | |
| | | | | | FLIGAVNLPAAALGMLFGGILMKRFVFSLQAIPRIA | |
| | | | | | TTIITISMILCVPLFFMGCSTPTVAEVYPPSTSSSIH | |
| | | | | | PQSPACRRDCSCPDSIFHPVCGDNGIEYLSPCHA | |
| | | | | | GCSNINMSSATSKQLIYLNCSCVTGGSASAKTGS | |
| | | | | | CPVPCAHFLLPAIFLISFVSLIACISHNPLYMMVLR | |
| | | | | | VVNQEEKSFAIGVQFLLMRLLAWLPSPALYGLTID | |
| | | | | | HSCIRWNSLCLGRRGACAYYDNDALRDRYLGLQ | |
| | | | | | MGYKALGMLLLCFISWRVKKNKEYNVQKAAGLI | |
| 260 | JHUO | NIV | 1.30072023 | SLCO2 | MGLLPKLGASQGSDTSTSRAGRCARSVFGNIKVF | Core Human ORF Only |
| | 6988 | O_ | 6 | A1 | VLCQGLLQLCQLLYSAYFKSSLTTIEKRFGLSSSS | |
| | | RFS | | | SGLISSLNEISNAILIIFVSYFGSRVHRPRLIGIGGLF | |
| | | | | | LAAGAFILTLPHFLSEPYQYTLASTGNNSRLQAEL | |
| | | | | | CQKHWQDLPPSKCHSTTQNPQKETSSMWGLMV | |
| | | | | | VAQLLAGIGTVPIQPFGISYVDDFSEPSNSPLYISIL | |
| | | | | | FAISVFGPAFGYLLGSVMLQIFVDYGRVNTAAVNL | |
| | | | | | VPGDPRWIGAWWLGLLISSALLVLTSFPFFFFPRA | |
| | | | | | MPIGAKRAPATADEARKLEEAKSRGSLVDFIKRFP | |
| | | | | | CIFLRLLMNSLFVLVVLAQCTFSSVIAGLSTFLNKF | |
| | | | | | LEKQYGTSAAYANFLIGAVNLPAAALGMLFGGILM | |
| | | | | | KRFVFSLQAIPRIATTIITISMILCVPLFFMGCSTPTV | |
| | | | | | AEVYPPSTSSSIHPQSPACRRDCSCPDSIFHPVC | |
| | | | | | GDNGIEYLSPCHAGCSNINMSSATSKQLIYLNCSC | |
| | | | | | VTGGSASAKTGSCPVPCAHFLLPAIFLISFVSLIACI | |
| | | | | | SHNPLYMMVLRVVNQEEKSFAIGVQFLLMRLLAW | |
| | | | | | LPSPALYGLTIDHSCIRWNSLCLGRRGACAYYDN | |
| | | | | | DALRDRYLGLQMGYKALGMLLLCFISWRVKKNKE | |
| | | | | | YNVQKAAGLI | |
| 261 | JHUO | NIV | 0.03455805 | CDC23 | MSPILGYWKIKGLVQPTRLLLEYLEEKYEEHLYER | Full Literal Sequence- |
| | 8372 | O_ | 7 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVPVAVTAAVAPVLS | |
| | | | | | INSDFSDLREIKKQLLLIAGLTRERGLLHSSKWSAE | |
| | | | | | LAFSLPALPLAELQPPPPITEEDAQDMDAYTLAKA | |
| | | | | | YFDVKEYDRAAHFLHGCNSKKAYFLYMYSRYLS | |
| | | | | | GEKKKDDETVDSLGPLEKGQVKNEALRELRVELS | |
| | | | | | KKHQARELDGFGLYLYGVVLRKLDLVKEAIDVFVE | |
| | | | | | ATHVLPLHWGAWLELCNLITDKEMLKFLSLPDTW | |
| | | | | | MKEFFLAHIYTELQLIEEALQKYQNLIDVGFSKSSY | |
| | | | | | IVSQIAVAYHNIRDIDKALSIFNELRKQDPYRIENM | |
| | | | | | DTFSNLLYVRSMKSELSYLAHNLCEIDKYRVETCC | |
| | | | | | VIGNYYSLRSQHEKAALYFQRALKLNPRYLGAWT | |
| | | | | | LMGHEYMEMKNTSAAIQAYRHAIEVNKRDYRAW | |
| | | | | | YGLGQTYEILKMPFYCLYYYRRAHQLRPNDSRML | |
| | | | | | VALGECYEKLNQLVEAKKCYWRAYAVGDVEKMA | |
| | | | | | LVKLAKLHEQLTESEQAAQCYIKYIQDIYSCGEIVE | |
| | | | | | HLEESTAFRYLAQYYFKCKLWDEASTCAQKCCAF | |
| | | | | | NDTREEGKALLRQILQLRNQGETPTTEVPAPFFLP | |
| | | | | | ASLSANNTPTRRVSPLNLSSVTP | |
| 262 | JHUO | NIV | 0.03455805 | CDC23 | MVPVAVTAAVAPVLSINSDFSDLREIKKQLLLIAGL | Core Human ORF Only |
| | 8372 | O_ | 7 | | TRERGLLHSSKWSAELAFSLPALPLAELQPPPPIT | |
| | | RFS | | | EEDAQDMDAYTLAKAYFDVKEYDRAAHFLHGCN | |
| | | | | | SKKAYFLYMYSRYLSGEKKKDDETVDSLGPLEKG | |
| | | | | | QVKNEALRELRVELSKKHQARELDGFGLYLYGVV | |
| | | | | | LRKLDLVKEAIDVFVEATHVLPLHWGAWLELCNLI | |
| | | | | | TDKEMLKFLSLPDTWMKEFFLAHIYTELQLIEEAL | |
| | | | | | QKYQNLIDVGFSKSSYIVSQIAVAYHNIRDIDKALSI | |
| | | | | | FNELRKQDPYRIENMDTFSNLLYVRSMKSELSYL | |
| | | | | | AHNLCEIDKYRVETCCVIGNYYSLRSQHEKAALYF | |
| | | | | | QRALKLNPRYLGAWTLMGHEYMEMKNTSAAIQA | |
| | | | | | YRHAIEVNKRDYRAWYGLGQTYEILKMPFYCLYY | |
| | | | | | YRRAHQLRPNDSRMLVALGECYEKLNQLVEAKK | |
| | | | | | CYWRAYAVGDVEKMALVKLAKLHEQLTESEQAA | |
| | | | | | QCYIKYIQDIYSCGEIVEHLEESTAFRYLAQYYFKC | |
| | | | | | KLWDEASTCAQKCCAFNDTREEGKALLRQILQLR | |
| | | | | | NQGETPTTEVPAPFFLPASLSANNTPTRRVSPLN | |
| | | | | | LSSVTP | |
| 263 | JHUO | NIV | 1.29638446 | HIST1H | MSPILGYWKIKGLVQPTRLLLEYLEEKYEEHLYER | Full Literal Sequence- |
| | 9262 | O_ | 7 | 3A | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMARTKQTARKSTGG | |
| | | | | | KAPRKQLATKAARKSAPATGGVKKPHRYRPGTV | |
| | | | | | ALREIRRYQKSTELLIRKLPFQRLVREIAQDFKTDL | |
| | | | | | RFQSSAVMALQEACEAYLVGLFEDTNLCAIHAKR | |
| | | | | | VTIMPKDIQLARRIRGERA | |
| 264 | JHUO | NIV | 1.2963844 | HIST1H | MARTKQTARKSTGGKAPRKQLATKAARKSAPAT | Core Human ORF Only |
| | 9262 | O_ | 67 | 3A | GGVKKPHRYRPGTVALREIRRYQKSTELLIRKLPF | |
| | | RFS | | | QRLVREIAQDFKTDLRFQSSAVMALQEACEAYLV | |
| | | | | | GLFEDTNLCAIHAKRVTIMPKDIQLARRIRGERA | |
| 265 | JHUO | NIV | 0.15069254 | HIST1H | MSPILGYWKIKGLVQPTRLLLEYLEEKYEEHLYER | Full Literal Sequence- |
| | 9262 | O_ | 5 | 3A | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMARTKQTARKSTGG | |
| | | | | | KAPRKQLATKAARKSAPATGGVKKPHRYRPGTV | |
| | | | | | ALREIRRYQKSTELLIRKLPFQRLVREIAQDFKTDL | |
| | | | | | RFQSSAVMALQEACEAYLVGLFEDTNLCAIHAKR | |
| | | | | | VTIMPKDIQLARRIRGERA | |
| 266 | JHUO | NIV | 0.15069254 | HIST1H | MARTKQTARKSTGGKAPRKQLATKAARKSAPAT | Core Human ORF Only |
| | 9262 | O_ | 5 | 3A | GGVKKPHRYRPGTVALREIRRYQKSTELLIRKLPF | |
| | | RFS | | | QRLVREIAQDFKTDLRFQSSAVMALQEACEAYLV | |
| | | | | | GLFEDTNLCAIHAKRVTIMPKDIQLARRIRGERA | |
| 267 | JHUO | NIV | 0.68824067 | KJ9041 | MSPILGYWKIKGLVQPTRLLLEYLEEKYEEHLYER | Full Literal Sequence- |
| | 9555 | O_ | 6 | 42_frag | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMHSTPANPVEAVVQ | |
| | | | | | RAGFVASQASLRPWKQDRRAGDPWPRTHEFGY | |
| | | | | | PEASLQASACKKKKSKDKKRKREDEETQLDTLES | |
| | | | | | GEQ | |
| 268 | JHUO | NIV | 0.68824067 | KJ9041 | MHSTPANPVEAVVQRAGFVASQASLRPWKQDR | Core Human ORF Only |
| | 9555 | O_ | 6 | 42_frag | RAGDPWPRTHEFGYPEASLQASACKKKKSKDKK | |
| | | RFS | | | RKREDEETQLDTLESGEQ | |
| 269 | JHUO | NIV | 0.03492676 | PNPLA | MSPILGYWKIKGLVQPTRLLLEYLEEKYEEHLYER | Full Literal Sequence- |
| | 9765 | O_ | 2 | 3 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMYDAERGWSLSFAG | |
| | | | | | CGFLGFYHVGATRCLSEHAPHLLRDARMLFGAS | |
| | | | | | AGALHCVGVLSGIPLEQTLQVLSDLVRKARSRNIG | |
| | | | | | IFHPSFNLSKFLRQGLGKCLPANVHQLISGKIGISL | |
| | | | | | TRVSDGENVLVSDFRSKDEVVDALVCSCFMPFY | |
| | | | | | SGLIPPSFRGVRYVDGGVSDNVPFIDAKTTITVSP | |
| | | | | | FYGEYDICPKVKSTNFLHVDITKLSLRLCTGNLYLL | |
| | | | | | SRAFVPPDLKVLGEICLRGYLDAFRFLEEKGICNR | |
| | | | | | PQPGLKSSSEGMDPEVAMPSWANMSLDSSPES | |
| | | | | | AALAVRLEGDELLDHLRLSILPWDESILDTLSPRLA | |
| | | | | | TALSEEMKDKGGYMSKICNLLPIRIMSYVMLPCTL | |
| | | | | | PVESAIAIVQRLVTWLPDMPDDVLWLQVWTSQVF | |
| | | | | | TRVLMCLLPASRSQMPVSSQQASPCTPEQDWPC | |
| | | | | | WTPCSPEGCPAETKAEATPRSILRSSLNFFLGNK | |
| | | | | | VPAGAEGLSTFPSFSLEKSL | |
| 270 | JHUO | NIV | 0.03492676 | PNPLA | MYDAERGWSLSFAGCGFLGFYHVGATRCLSEHA | Core Human ORF Only |
| | 9765 | O_ | 2 | 3 | PHLLRDARMLFGASAGALHCVGVLSGIPLEQTLQ | |
| | | RFS | | | VLSDLVRKARSRNIGIFHPSFNLSKFLRQGLGKCL | |
| | | | | | PANVHQLISGKIGISLTRVSDGENVLVSDFRSKDE | |
| | | | | | VVDALVCSCFMPFYSGLIPPSFRGVRYVDGGVSD | |
| | | | | | NVPFIDAKTTITVSPFYGEYDICPKVKSTNFLHVDI | |
| | | | | | TKLSLRLCTGNLYLLSRAFVPPDLKVLGEICLRGY | |
| | | | | | LDAFRFLEEKGICNRPQPGLKSSSEGMDPEVAMP | |
| | | | | | SWANMSLDSSPESAALAVRLEGDELLDHLRLSIL | |
| | | | | | PWDESILDTLSPRLATALSEEMKDKGGYMSKICN | |
| | | | | | LLPIRIMSYVMLPCTLPVESAIAIVQRLVTWLPDMP | |
| | | | | | DDVLWLQWVTSQVFTRVLMCLLPASRSQMPVSS | |
| | | | | | QQASPCTPEQDWPCWTPCSPEGCPAETKAEAT | |
| | | | | | PRSILRSSLNFFLGNKVPAGAEGLSTFPSFSLEKS | |
| | | | | | L | |
| 271 | JHU1 | NIV | 0.05454885 | MMP3 | MSPILGYWKIKGLVQPTRLLLEYLEEKYEEHLYER | Full Literal Sequence- |
| | 0228 | O_ | 9 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKSLPILLLLCVAVCS | |
| | | | | | AYPLDGAARGEDTSMNLVQKYLENYYDLKKDVK | |
| | | | | | QFVRRKDSGPVVKKIREMQKFLGLEVTGKLDSDT | |
| | | | | | LEVMRKPRCGVPDVGHFRTFPGIPKWRKTHLTY | |
| | | | | | RIVNYTPDLPKDAVDSAVEKALKVWEEVTPLTFS | |
| | | | | | RLYEGEADIMISFAVREHGDFYPFDGPGNVLAHA | |
| | | | | | YAPGPGINGDAHFDDDEQWTKDTTGTNLFLVAA | |
| | | | | | HEIGHSLGLFHSANTEALMYPLYHSLTDLTRFRLS | |
| | | | | | QDDINGIQSLYGPPPDSPETPLVPTEPVPPEPGTP | |
| | | | | | ANCDPALSFDAVSTLRGEILIFKDRHFWRKSLRKL | |
| | | | | | EPELHLISSFWPSLPSGVDAAYEVTSKDLVFIFKG | |
| | | | | | NQFWAIRGNEVRAGYPRGIHTLGFPPTVRKIDAAI | |
| | | | | | SDKEKNKTYFFVEDKYWRFDEKRNSMEPGFPKQ | |
| | | | | | IAEDFPGIDSKIDAVFEEFGFFYFFTGSSQLEFDPN | |
| | | | | | AKKVTHTLKSNSWLNC | |
| 272 | JHU1 | NIV | 0.05454885 | MMP3 | MKSLPILLLLCVAVCSAYPLDGAARGEDTSMNLV | Core Human ORF Only |
| | 0228 | O_ | 9 | | QKYLENYYDLKKDVKQFVRRKDSGPVVKKIREM | |
| | | RFS | | | QKFLGLEVTGKLDSDTLEVMRKPRCGVPDVGHF | |
| | | | | | RTFPGIPKWRKTHLTYRIVNYTPDLPKDAVDSAVE | |
| | | | | | KALKVWEEVTPLTFSRLYEGEADIMISFAVREHGD | |
| | | | | | FYPFDGPGNVLAHAYAPGPGINGDAHFDDDEQW | |
| | | | | | TKDTTGTNLFLVAAHEIGHSLGLFHSANTEALMYP | |
| | | | | | LYHSLTDLTRFRLSQDDINGIQSLYGPPPDSPETP | |
| | | | | | LVPTEPVPPEPGTPANCDPALSFDAVSTLRGEILI | |
| | | | | | FKDRHFWRKSLRKLEPELHLISSFWPSLPSGVDA | |
| | | | | | AYEVTSKDLVFIFKGNQFWAIRGNEVRAGYPRGI | |
| | | | | | HTLGFPPTVRKIDAAISDKEKNKTYFFVEDKYWRF | |
| | | | | | DEKRNSMEPGFPKQIAEDFPGIDSKIDAVFEEFGF | |
| | | | | | FYFFTGSSQLEFDPNAKKVTHTLKSNSWLNC | |
| 273 | JHU1 | NIV | 0.35950373 | LINC01 | MSPILGYWKIKGLVQPTRLLLEYLEEKYEEHLYER | Full Literal Sequence- |
| | 0283 | O_ | 4 | 554 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTPCLESFCQRAKD | |
| | | | | | CKQNIPLPFLIQDWPLSESKPQGQDFWTPQPLQA | |
| | | | | | AAPSHEFQQVALHPARTRLASRPQGGRLMSSRE | |
| | | | | | VGLRAPRCSCGKRKR | |
| 274 | JHU1 | NIV | 0.35950373 | LINC01 | MTPCLESFCQRAKDCKQNIPLPFLIQDWPLSESK | Core Human ORF Only |
| | 0283 | O_ | 4 | 554 | PQGQDFWTPQPLQAAAPSHEFQQVALHPARTRL | |
| | | RFS | | | ASRPQGGRLMSSREVGLRAPRCSCGKRKR | |
| 275 | JHU1 | NIV | 0.55591174 | TNFSF | MSPILGYWKIKGLVQPTRLLLEYLEEKYEEHLYER | Full Literal Sequence- |
| | 1509 | O_ | 7 | 11 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDPNRISEDGTHCIY | |
| | | | | | RILRLHENADFQDTTLESQDTKLIPDSCRRIKQAF | |
| | | | | | QGAVQKELQHIVGSQHIRAEKAMVDGSWLDLAK | |
| | | | | | RSKLEAQPFAHLTINATDIPSGSHKVSLSSWYHD | |
| | | | | | RGWAKISNMTFSNGKLIVNQDGFYYLYANICFRH | |
| | | | | | HETSGDLATEYLQLMVYVTKTSIKIPSSHTLMKGG | |
| | | | | | STKYWSGNSEFHFYSINVGGFFKLRSGEEISIEVS | |
| | | | | | NPSLLDPDQDATYFGAFKVRDID | |
| 276 | JHU1 | NIV | 0.55591174 | TNFSF | MDPNRISEDGTHCIYRILRLHENADFQDTTLESQD | Core Human ORF Only |
| | 1509 | O_ | 7 | 11 | TKLIPDSCRRIKQAFQGAVQKELQHIVGSQHIRAE | |
| | | RFS | | | KAMVDGSWLDLAKRSKLEAQPFAHLTINATDIPS | |
| | | | | | GSHKVSLSSWYHDRGWAKISNMTFSNGKLIVNQ | |
| | | | | | DGFYYLYANICFRHHETSGDLATEYLQLMVYVTK | |
| | | | | | TSIKIPSSHTLMKGGSTKYWSGNSEFHFYSINVG | |
| | | | | | GFFKLRSGEEISIEVSNPSLLDPDQDATYFGAFKV | |
| | | | | | RDID | |
| 277 | JHU1 | NIV | 0.71036684 | SLC22 | MSPILGYWKIKGLVQPTRLLLEYLEEKYEEHLYER | Full Literal Sequence- |
| | 1583 | O_ | 1 | A12 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEWTAARARPLVMT | |
| | | | | | LNSLGFSFGHGLTAAVAYGVRDWTLLQLVVSVPF | |
| | | | | | FLCFLYSVWVLAESARWLLTTGRLDWGLQELWRV | |
| | | | | | AAINGKGAVQDTLTPEVLLSAMREELSMGQPPAS | |
| | | | | | LGTLLRMPGLRFRTCISTLCWFAFGFTFFGLALDL | |
| | | | | | QALGSNIFLLQMFIGVVDIPAKMGALLLLSHLGRR | |
| | | | | | PTLAASLLLAGLCILANTLVPHEMGALRSALAVLG | |
| | | | | | LGGVGAAFTCITIYSSELFPTVLRMTAVGLGQMAA | |
| | | | | | RGGAILGPLVRLLGVHGPWLPLLVYGTVPVLSGL | |
| | | | | | AALLLPETQSLPLPDTIQDVQNQAVKKATHGTLG | |
| | | | | | NSVLKSTQF | |
| 278 | JHU1 | NIV | 0.71036684 | SLC22 | MEWTAARARPLVMTLNSLGFSFGHGLTAAVAYG | Core Human ORF Only |
| | 1583 | O_ | 1 | A12 | VRDWTLLQLVVSVPFFLCFLYSWWLAESARWLLT | |
| | | RFS | | | TGRLDWGLQELWRVAAINGKGAVQDTLTPEVLLS | |
| | | | | | AMREELSMGQPPASLGTLLRMPGLRFRTCISTLC | |
| | | | | | WFAFGFTFFGLALDLQALGSNIFLLQMFIGVVDIP | |
| | | | | | AKMGALLLLSHLGRRPTLAASLLLAGLCILANTLVP | |
| | | | | | HEMGALRSALAVLGLGGVGAAFTCITIYSSELFPT | |
| | | | | | VLRMTAVGLGQMAARGGAILGPLVRLLGVHGPW | |
| | | | | | LPLLVYGTVPVLSGLAALLLPETQSLPLPDTIQDV | |
| | | | | | QNQAVKKATHGTLGNSVLKSTQF | |
| 279 | JHU1 | NIV | 1.85398815 | TTC39 | MSPILGYWKIKGLVQPTRLLLEYLEEKYEEHLYER | Full Literal Sequence- |
| | 2078 | O_ | 8 | B | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMALLGSRAELEADED | |
| | | | | | VFEDALETISISSHSDMATSSLHFASCDTQQAPRQ | |
| | | | | | RGASTVSSSSSTKVDLKSGLEECAVALNLFLSNK | |
| | | | | | FTDALELLRPWAKESMYHALGYSTIVVLQAVLTFE | |
| | | | | | QQDIQNGISAMKDALQTCQKYRKKYTVVESFSSL | |
| | | | | | LSRGSLEQLSEEEMHAEICYAECLLQKAALTFVQ | |
| | | | | | DENMINFIKGGLKIRTSYQIYKECLSILHEIQKNKLQ | |
| | | | | | QEFFYEFEGGVKLGSGAFNLMLSLLPARIIRLLEFI | |
| | | | | | GFSGNRELGLLQLREGASGRSMRSALCCLTILAF | |
| | | | | | HTYISLILGTGEVNVAEAERLLAPFLQQFPNGSLV | |
| | | | | | LFYHARIELLKGNLEEAQEVFQKCVSVQEEWKQF | |
| | | | | | HHLCYWELMWINVFQQNWMQAYYYSDLLCKES | |
| | | | | | KWSKATYVFLKAAILSMLPEEDVVATNENVVTLFR | |
| | | | | | QVDSLKQRIAGKSIPTEKFAVRKARRYSASLPAPV | |
| | | | | | KLILPALEMMYVWNGFSIVSKRKDLSENLLVTVEK | |
| | | | | | AEAALQSQNFNSFSVDDECLVKLLKGCCLKNLQR | |
| | | | | | PLQAELCYNHVVESEKLLKYDHYLVPFTLFELASL | |
| | | | | | YKSQGEIDKAIKFLETARNNYKDYSLESRLHFRIQ | |
| | | | | | AALHLWRKPSSD | |
| 280 | JHU1 | NIV | 1.85398815 | TTC39 | MALLGSRAELEADEDVFEDALETISISSHSDMATS | Core Human ORF Only |
| | 2078 | O_ | 8 | B | SLHFASCDTQQAPRQRGASTVSSSSSTKVDLKS | |
| | | RFS | | | GLEECAVALNLFLSNKFTDALELLRPWAKESMYH | |
| | | | | | ALGYSTIVVLQAVLTFEQQDIQNGISAMKDALQTC | |
| | | | | | QKYRKKYTVVESFSSLLSRGSLEQLSEEEMHAEI | |
| | | | | | CYAECLLQKAALTFVQDENMINFIKGGLKIRTSYQI | |
| | | | | | YKECLSILHEIQKNKLQQEFFYEFEGGVKLGSGAF | |
| | | | | | NLMLSLLPARIIRLLEFIGFSGNRELGLLQLREGAS | |
| | | | | | GRSMRSALCCLTILAFHTYISLILGTGEVNVAEAER | |
| | | | | | LLAPFLQQFPNGSLVLFYHARIELLKGNLEEAQEV | |
| | | | | | FQKCVSVQEEWKQFHHLCYWELMWINVFQQNW | |
| | | | | | MQAYYYSDLLCKESKWSKATYVFLKAAILSMLPE | |
| | | | | | EDVVATNENVVTLFRQVDSLKQRIAGKSIPTEKFA | |
| | | | | | VRKARRYSASLPAPVKLILPALEMMYVWNGFSIV | |
| | | | | | SKRKDLSENLLVTVEKAEAALQSQNFNSFSVDDE | |
| | | | | | CLVKLLKGCCLKNLQRPLQAELCYNHVVESEKLL | |
| | | | | | KYDHYLVPFTLFELASLYKSQGEIDKAIKFLETARN | |
| | | | | | NYKDYSLESRLHFRIQAALHLWRKPSSD | |
| 281 | JHU1 | NIV | 0.7984539 | CCNT1 | MSPILGYWKIKGLVQPTRLLLEYLEEKYEEHLYER | Full Literal Sequence- |
| | 2079 | O_ | 6 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEGERKNNNKRWYF | |
| | | | | | TREQLENSPSRRFGVDPDKELSYRQQAANLLQD | |
| | | | | | MGQRLNVSQLTINTAIVYMHRFYMIQSFTQFPGN | |
| | | | | | SVAPAALFLAAKVEEQPKKLEHVIKVAHTCLHPQE | |
| | | | | | SLPDTRSEAYLQQVQDLVILESIILQTLGFELTIDHP | |
| | | | | | HTHVVKCTQLVRASKDLAQTSYFMATNSLHLTTF | |
| | | | | | SLQYTPPVVACVCIHLACKWSNWEIPVSTDGKH | |
| | | | | | WWEYVDATVTLELLDELTHEFLQILEKTPNRLKRI | |
| | | | | | WNWRACEAAKKTKADDRGTDEKTSEQTILNMIS | |
| | | | | | QSSSDTTIAGLMSMSTSTTSAVPSLPVSEESSSN | |
| | | | | | LTSVEMLPGKRWLSSQPSFKLEPTQGHRTSENL | |
| | | | | | ALTGVDHSLPQDGSNAFISQKQNSKSVPSAKVSL | |
| | | | | | KEYRAKHAEELAAQKRQLENMEANVKSQYAYAA | |
| | | | | | QNLLSHHDSHSSVILKMPIEGSENPERPFLEKADK | |
| | | | | | TALKMRIPVAGGDKAASSKPEEIKMRIKVHAAADK | |
| | | | | | HNSVEDSVTKSREHKEKHKTHPSNHHHHHNHHS | |
| | | | | | HKHSHSQLPVGTGNKRPGDPKHSSQTSNLAHKT | |
| | | | | | YSLSSSFSSSSSTRKRGPSEETGGAVFDHPAKIA | |
| | | | | | KSTKSSSLNFSFPSLPTMGQMPGHSSDTSGLSFS | |
| | | | | | QPSCKTRVPHSKLDKGPTGANGHNTTQTIDYQD | |
| | | | | | TVNMLHSLLSAQGVQPTQPTAFEFVRPYSDYLNP | |
| | | | | | RSGGISSRSGNTDKPRPPPLPSEPPPPLPPLPK | |
| 282 | JHU1 | NIV | 0.7984539 | CCNT1 | MEGERKNNNKRWYFTREQLENSPSRRFGVDPD | Core Human ORF Only |
| | 2079 | O_ | 6 | | KELSYRQQAANLLQDMGQRLNVSQLTINTAIVYM | |
| | | RFS | | | HRFYMIQSFTQFPGNSVAPAALFLAAKVEEQPKK | |
| | | | | | LEHVIKVAHTCLHPQESLPDTRSEAYLQQVQDLVI | |
| | | | | | LESIILQTLGFELTIDHPHTHVVKCTQLVRASKDLA | |
| | | | | | QTSYFMATNSLHLTTFSLQYTPPVVACVCIHLACK | |
| | | | | | WSNWEIPVSTDGKHVWVEYVDATVTLELLDELTH | |
| | | | | | EFLQILEKTPNRLKRIWNWRACEAAKKTKADDRG | |
| | | | | | TDEKTSEQTILNMISQSSSDTTIAGLMSMSTSTTS | |
| | | | | | AVPSLPVSEESSSNLTSVEMLPGKRWLSSQPSFK | |
| | | | | | LEPTQGHRTSENLALTGVDHSLPQDGSNAFISQK | |
| | | | | | QNSKSVPSAKVSLKEYRAKHAEELAAQKRQLEN | |
| | | | | | MEANVKSQYAYAAQNLLSHHDSHSSVILKMPIEG | |
| | | | | | SENPERPFLEKADKTALKMRIPVAGGDKAASSKP | |
| | | | | | EEIKMRIKVHAAADKHNSVEDSVTKSREHKEKHK | |
| | | | | | THPSNHHHHHNHHSHKHSHSQLPVGTGNKRPG | |
| | | | | | DPKHSSQTSNLAHKTYSLSSSFSSSSSTRKRGPS | |
| | | | | | EETGGAVFDHPAKIAKSTKSSSLNFSFPSLPTMG | |
| | | | | | QMPGHSSDTSGLSFSQPSCKTRVPHSKLDKGPT | |
| | | | | | GANGHNTTQTIDYQDTVNMLHSLLSAQGVQPTQ | |
| | | | | | PTAFEFVRPYSDYLNPRSGGISSRSGNTDKPRPP | |
| | | | | | PLPSEPPPPLPPLPK | |
| 283 | JHU1 | NIV | 0.05950564 | PSMG1 | MSPILGYWKIKGLVQPTRLLLEYLEEKYEEHLYER | Full Literal Sequence- |
| | 2087 | O_ | 5 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAATFFGEVVKAPC | |
| | | | | | RAGTEDEEEEEEGRRETPEDREVRLQLARKREV | |
| | | | | | RLLRRQTKTSLEVSLLEKYPCSKFIIAIGNNAVAFL | |
| | | | | | SSFVMNSGVWEEVGCAKLWNEWCRTTDTTHLS | |
| | | | | | STEAFCVFYHLKSNPSVFGSCPRKNMQITILTCRH | |
| | | | | | VTDYKTSESTGSLPSPFLRALKTQNFKDSACCPL | |
| | | | | | LEQPNIVHDLPAAVLSYCQVWKIPAILYLCYTDVM | |
| | | | | | KLDLITVEAFKPILSTRSLKGLVKNIPQSTEILKK | |
| | | | | | LMTTNEIQSNIYT | |
| 284 | JHU1 | NIV | 0.05950564 | PSMG1 | MAATFFGEVVKAPCRAGTEDEEEEEEGRRETPE | Core Human ORF Only |
| | 2087 | O_ | 5 | | DREVRLQLARKREVRLLRRQTKTSLEVSLLEKYP | |
| | | RFS | | | CSKFIIAIGNNAVAFLSSFVMNSGVWEEVGCAKL | |
| | | | | | WNEWCRTTDTTHLSSTEAFCVFYHLKSNPSVFG | |
| | | | | | SCPRKNMQITILTCRHVTDYKTSESTGSLPSPFLR | |
| | | | | | ALKTQNFKDSACCPLLEQPNIVHDLPAAVLSYCQ | |
| | | | | | VWKIPAILYLCYTDVMKLDLITVEAFKPILSTRSLK | |
| | | | | | GLVKNIPQSTEILKKLMTTNEIQSNIYT | |
| 285 | JHU1 | NIV | 0.92607689 | DEPTO | MSPILGYWKIKGLVQPTRLLLEYLEEKYEEHLYER | Full Literal Sequence- |
| | 2280 | o_ | 9 | R | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEEGGSTGSAGSDS | |
| | | | | | STSGSGGAQQRELERMAEVLVTGEQLRLRLHEE | |
| | | | | | KVIKDRRHHLKTYPNCFVAKELIDWLIEHKEASDR | |
| | | | | | ETAIKLMQKLADRGIIHHVCDEHKEFKDVKLFYRF | |
| | | | | | RKDDGTFPLDNEVKAFMRGQRLYEKLMSPENTL | |
| | | | | | LQPREEEGVKYERTFMASEFLDWLVQEGEATTR | |
| | | | | | KEAEQLCHRLMEHGIIQHVSSKHPFVDSNLLYQF | |
| | | | | | RMNFRRRRRLMELLNEKSPSSQETHDSPFCLRK | |
| | | | | | QSHDNRKSTSFMSVSPSKEIKIVSAVRRSSMSSC | |
| | | | | | GSSGYFSSSPTLSSSPPVLCNPKSVLKRPVTSEE | |
| | | | | | LLTPGAPYARKTFTIVGDAVGWGFVVRGSKPCHI | |
| | | | | | QAVDPSGPAAAAGMKVCQFVVSVNGLNVLHVDY | |
| | | | | | RTVNNLILTGPRTIVMEVMEELEC | |
| 286 | JHU1 | NIV | 0.92607689 | DEPTO | MEEGGSTGSAGSDSSTSGSGGAQQRELERMAE | Core Human ORF Only |
| | 2280 | O_ | 9 | R | VLVTGEQLRLRLHEEKVIKDRRHHLKTYPNCFVA | |
| | | RFS | | | KELIDWLIEHKEASDRETAIKLMQKLADRGIIHHVC | |
| | | | | | DEHKEFKDVKLFYRFRKDDGTFPLDNEVKAFMR | |
| | | | | | GQRLYEKLMSPENTLLQPREEEGVKYERTFMAS | |
| | | | | | EFLDWLVQEGEATTRKEAEQLCHRLMEHGIIQHV | |
| | | | | | SSKHPFVDSNLLYQFRMNFRRRRRLMELLNEKS | |
| | | | | | PSSQETHDSPFCLRKQSHDNRKSTSFMSVSPSK | |
| | | | | | EIKIVSAVRRSSMSSCGSSGYFSSSPTLSSSPPVL | |
| | | | | | CNPKSVLKRPVTSEELLTPGAPYARKTFTIVGDAV | |
| | | | | | GWGFVVRGSKPCHIQAVDPSGPAAAAGMKVCQ | |
| | | | | | FVVSVNGLNVLHVDYRTVNNLILTGPRTIVMEVME | |
| | | | | | ELEC | |
| 287 | JHU1 | NIV | 0.43683504 | MS4A3 | MSPILGYWKIKGLVQPTRLLLEYLEEKYEEHLYER | Full Literal Sequence- |
| | 4571 | O_ | 4 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNIASATIALVGTAFL | |
| | | | | | SLNIAVNIQSLRSCHSSSESPDLCNYMGSISNGM | |
| | | | | | VSLLLILTLLELCVTISTIAMWCNANCCNSREEISS | |
| | | | | | PPNSV | |
| 288 | JHU1 | NIV | 0.43683504 | MS4A3 | MNIASATIALVGTAFLSLNIAVNIQSLRSCHSSSES | Core Human ORF Only |
| | 4571 | O_ | 4 | | PDLCNYMGSISNGMVSLLLILTLLELCVTISTIAMW | |
| | | RFS | | | CNANCCNSREEISSPPNSV | |
| 289 | JHU1 | NIV | 0.66771592 | OLFM1 | MSPILGYWKIKGLVQPTRLLLEYLEEKYEEHLYER | Full Literal Sequence- |
| | 4575 | O_ | 8 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPGRWRWQRDMHP | |
| | | | | | ARKLLSLLFLILMGTELTQVLPTNPEESWQVYSSA | |
| | | | | | QDSEGRCICTVVAPQQTMCSRDARTKQLRQLLE | |
| | | | | | KVQNMSQSIEVLDRRTQRDLQYVEKMENQMKGL | |
| | | | | | ESKFKQVEESHKQHLARQFKG | |
| 290 | JHU1 | NIV | 0.66771592 | OLFM1 | MPGRWRWQRDMHPARKLLSLLFLILMGTELTQV | Core Human ORF Only |
| | 4575 | O_ | 8 | | LPTNPEESWQVYSSAQDSEGRCICTVVAPQQTM | |
| | | RFS | | | CSRDARTKQLRQLLEKVQNMSQSIEVLDRRTQR | |
| | | | | | DLQYVEKMENQMKGLESKFKQVEESHKQHLARQ | |
| | | | | | FKG | |
| 291 | JHU1 | NIV | 1.84128223 | TMEM1 | MSPILGYWKIKGLVQPTRLLLEYLEEKYEEHLYER | Full Literal Sequence- |
| | 5273 | O_ | 5 | 16 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKHTQSGQSTSPLVI | |
| | | | | | DFTCRVCQMAFVFSSLIPLLLMTPVFCLGNTSECF | |
| | | | | | QNFSQSHKCILMHSPPSAMAELPPSANTSVCSTL | |
| | | | | | YFYGIAIFLGSFVLSLLTIMVLLIRAQTLYKKFVKST | |
| | | | | | GFLGSEQWAVIHIVDQRVRFYPVAFFCCWGPAVI | |
| | | | | | LMIIKLTKPQDTKLRMALYVLQALTATSQGLLNCG | |
| | | | | | VYGWTQHKFHQLKQEARRDADTQTPLLCSQKRF | |
| | | | | | YSRGLNSLESTLTFPASTSTIF | |
| 292 | JHU1 | NIV | 1.84128223 | TMEM1 | MKHTQSGQSTSPLVIDFTCRVCQMAFVFSSLIPLL | Core Human ORF Only |
| | 5273 | O_ | 5 | 16 | LMTPVFCLGNTSECFQNFSQSHKCILMHSPPSAM | |
| | | RFS | | | AELPPSANTSVCSTLYFYGIAIFLGSFVLSLLTIMVL | |
| | | | | | LIRAQTLYKKFVKSTGFLGSEQWAVIHIVDQRVRF | |
| | | | | | YPVAFFCCWGPAVILMIIKLTKPQDTKLRMALYVL | |
| | | | | | QALTATSQGLLNCGVYGWTQHKFHQLKQEARRD | |
| | | | | | ADTQTPLLCSQKRFYSRGLNSLESTLTFPASTSTI | |
| | | | | | F | |
| 293 | JHU1 | NIV | 0.02339660 | NDUFA | MSPILGYWKIKGLVQPTRLLLEYLEEKYEEHLYER | Full Literal Sequence- |
| | 6454 | O_ | 2 | 5 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGVLKKTTGLVGLA | |
| | | | | | VCNTPHERLRILYTKILDVLEEIPKNAAYRKYTEQII | |
| | | | | | NEKLAMVKAEPDVKKLEDQLQGGQLEEVILQAEH | |
| | | | | | ELNLARKMREWKLWEPLVEEPPADQWKWPI | |
| 294 | JHU1 | NIV | 0.02339660 | NDUFA | MAGVLKKTTGLVGLAVCNTPHERLRILYTKILDVL | Core Human ORF Only |
| | 6454 | O_ | 2 | 5 | EEIPKNAAYRKYTEQIINEKLAMVKAEPDVKKLED | |
| | | RFS | | | QLQGGQLEEVILQAEHELNLARKMREWKLWEPL | |
| | | | | | VEEPPADQWKWPI | |
| 295 | JHU1 | NIV | 0.54354796 | NDUFA | MSPILGYWKIKGLVQPTRLLLEYLEEKYEEHLYER | Full Literal Sequence- |
| | 6454 | O_ | 1 | 5 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGVLKKTTGLVGLA | |
| | | | | | VCNTPHERLRILYTKILDVLEEIPKNAAYRKYTEQII | |
| | | | | | NEKLAMVKAEPDVKKLEDQLQGGQLEEVILQAEH | |
| | | | | | ELNLARKMREWKLWEPLVEEPPADQWKWPI | |
| 296 | JHU1 | NIV | 0.54354796 | NDUFA | MAGVLKKTTGLVGLAVCNTPHERLRILYTKILDVL | Core Human ORF Only |
| | 6454 | O_ | 1 | 5 | EEIPKNAAYRKYTEQIINEKLAMVKAEPDVKKLED | |
| | | RFS | | | QLQGGQLEEVILQAEHELNLARKMREWKLWEPL | |
| | | | | | VEEPPADQWKWPI | |
| 297 | JHU1 | NIV | 1.63415485 | SCUBE | MSPILGYWKIKGLVQPTRLLLEYLEEKYEEHLYER | Full Literal Sequence- |
| | 7760 | O_ | 8 | 1 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGAAAVRWHLCVLL | |
| | | | | | ALGTRGRLAGGSGLPGSVDVDECSEGTDDCHID | |
| | | | | | AICQNTPKSYKCLCKPGYKGEGKQCEDIDECEND | |
| | | | | | YYNGGCVHECINIPGNYRCTCFDGFMLAHDGHN | |
| | | | | | CLDVDECQDNNGGCQQICVNAMGSYECQCHSG | |
| | | | | | FFLSDNQHTCIHRSNEGMNCMNKDHGCAHICRE | |
| | | | | | TPKGGVACDCRPGFDLAQNQKDCTLTCNYGNG | |
| | | | | | GCQHSCEDTDTGPTCGCHQKYALHSDGRTCIET | |
| | | | | | CAVNNGGCDRTCKDTATGVRCSCPVGFTLQPDG | |
| | | | | | KTCKDINECLVNNGGCDHFCRNTVGSFECGCRK | |
| | | | | | GYKLLTDERTCQDIDECSFERTCDHICINSPGSFQ | |
| | | | | | CLCHRGYILYGTTHCGDVDECSMSNGSCDQGCV | |
| | | | | | NTKGSYECVCPPGRRLHWNGKDCVETGKCLSR | |
| | | | | | AKTSPRAQLSCSKAGGVESCFLSCPAHTLFVPDS | |
| | | | | | ENSYVLSCGVPGPQGKALQKRNGTSSGLGPSCS | |
| | | | | | DAPTTPIKQKARFKIRDAKCHLRPHSQARAKETA | |
| | | | | | RQPLLDHCHVTFVTLKCDSSKKRRRGRKSPSKE | |
| | | | | | VSHITAEFEIETKMEEASDTCEADCLRKRAEQSLQ | |
| | | | | | AAIKTLRKSIGRQQFYVQVSGTEYEVAQRPAKAL | |
| | | | | | EGQGACGAGQVLQDSKCVACGPGTHFGGELGQ | |
| | | | | | CVSCMPGTYQDMEGQLSCTPCPSSDGLGLPGA | |
| | | | | | RNVSECGGQCSPGFFSADGFKPCQACPVGTYQ | |
| | | | | | PEPGRTGCFPCGGGLLTKHEGTTSFQDCEAKVH | |
| | | | | | CSPGHHYNTTTHRCIRCPVGTYQPEFGQNHCITC | |
| | | | | | PGNTSTDFDGSTNVTHCKNQHCGGELGDYTGYI | |
| | | | | | ESPNYPGDYPANAECVWHIAPPPKRRILIVVPEIFL | |
| | | | | | PIEDECGDVLVMRKSASPTSITTYETCQTYERPIA | |
| | | | | | FTSRSRKLWIQFKSNEGNSGKGFQVPYVTYDED | |
| | | | | | YQQLIEDIVRDGRLYASENHQEILKDKKLIKALFDV | |
| | | | | | LAHPQNYFKYTAQESKEMFPRSFIKLLRSKVSRFL | |
| | | | | | RPYKNPAFLYKVVKASS | |
| 298 | JHU1 | NIV | 1.63415485 | SCUBE | MGAAAVRWHLCVLLALGTRGRLAGGSGLPGSVD | Core Human ORF Only |
| | 7760 | O_ | 8 | 1 | VDECSEGTDDCHIDAICQNTPKSYKCLCKPGYKG | |
| | | RFS | | | EGKQCEDIDECENDYYNGGCVHECINIPGNYRCT | |
| | | | | | CFDGFMLAHDGHNCLDVDECQDNNGGCQQICV | |
| | | | | | NAMGSYECQCHSGFFLSDNQHTCIHRSNEGMNC | |
| | | | | | MNKDHGCAHICRETPKGGVACDCRPGFDLAQNQ | |
| | | | | | KDCTLTCNYGNGGCQHSCEDTDTGPTCGCHQK | |
| | | | | | YALHSDGRTCIETCAVNNGGCDRTCKDTATGVR | |
| | | | | | CSCPVGFTLQPDGKTCKDINECLVNNGGCDHFC | |
| | | | | | RNTVGSFECGCRKGYKLLTDERTCQDIDECSFER | |
| | | | | | TCDHICINSPGSFQCLCHRGYILYGTTHCGDVDE | |
| | | | | | CSMSNGSCDQGCVNTKGSYECVCPPGRRLHWN | |
| | | | | | GKDCVETGKCLSRAKTSPRAQLSCSKAGGVESC | |
| | | | | | FLSCPAHTLFVPDSENSYVLSCGVPGPQGKALQK | |
| | | | | | RNGTSSGLGPSCSDAPTTPIKQKARFKIRDAKCH | |
| | | | | | LRPHSQARAKETARQPLLDHCHVTFVTLKCDSSK | |
| | | | | | KRRRGRKSPSKEVSHITAEFEIETKMEEASDTCE | |
| | | | | | ADCLRKRAEQSLQAAIKTLRKSIGRQQFYVQVSG | |
| | | | | | TEYEVAQRPAKALEGQGACGAGQVLQDSKCVAC | |
| | | | | | GPGTHFGGELGQCVSCMPGTYQDMEGQLSCTP | |
| | | | | | CPSSDGLGLPGARNVSECGGQCSPGFFSADGFK | |
| | | | | | PCQACPVGTYQPEPGRTGCFPCGGGLLTKHEGT | |
| | | | | | TSFQDCEAKVHCSPGHHYNTTTHRCIRCPVGTY | |
| | | | | | QPEFGQNHCITCPGNTSTDFDGSTNVTHCKNQH | |
| | | | | | CGGELGDYTGYIESPNYPGDYPANAECVWHIAPP | |
| | | | | | PKRRILIVVPEIFLPIEDECGDVLVMRKSASPTSI | |
| | | | | | TTYETCQTYERPIAFTSRSRKLWIQFKSNEGNSGK | |
| | | | | | GFQVPYVTYDEDYQQLIEDIVRDGRLYASENHQEI | |
| | | | | | LKDKKLIKALFDVLAHPQNYFKYTAQESKEMFPRS | |
| | | | | | FIKLLRSKVSRFLRPYK | |
| 299 | JHU1 | NIV | 1.04193386 | CDC27 | MSPILGYWKIKGLVQPTRLLLEYLEEKYEEHLYER | Full Literal Sequence- |
| | 8346 | O_ | 3 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTVLQEPVQAAIWQ | |
| | | | | | ALNHYAYRDAVFLAERLYAEVHSEEALFLLATCYY | |
| | | | | | RSGKAYKAYRLLKGHSCTTPQCKYLLAKCCVDLS | |
| | | | | | KLAEGEQILSGGVFNKQKSHDDIVTEFGDSACFTL | |
| | | | | | SLLGHVYCKTDRLAKGSECYQKSLSLNPFLWSPF | |
| | | | | | ESLCEIGEKPDPDQTFKFTSLQNFSNCLPNSCTT | |
| | | | | | QVPNHSLSHRQPETVLTETPQDTIELNRLNLESS | |
| | | | | | NSKYSLNTDSSVSYIDSAVISPDTVPLGTGTSILSK | |
| | | | | | QVQNKPKTGRSLLGGPAALSPLTPSFGILPLETPS | |
| | | | | | PGDGSYLQNYTNTPPVIDVPSTGAPSKKTFRVLQ | |
| | | | | | SVARIGQTGTKSVFSQSGNSREVTPILAQTQSSG | |
| | | | | | PQTSTTPQVLSPTITSPPNALPRRSSRLFTSDSST | |
| | | | | | TKENSKKLKMKFPPEIPNRKTKSKTNKGGITQPNI | |
| | | | | | NDSLEITKLDSSIISEGKISTITPQIQAFNLQKAAAE | |
| | | | | | GLMSLLREMGKGYLALCSYNCKEAINILSHLPSHH | |
| | | | | | YNTGVWLCQIGRAYFELSEYMQAERIFSEVRRIE | |
| | | | | | NYRVEGMEIYSTTLWHLQKDVALSVLSKDLTDMD | |
| | | | | | KNSPEAWCAAGNCFSLQREHDIAIKFFQRAIQVD | |
| | | | | | PNYAYAYTLLGHEFVLTEELDKALACFRNAIRVNP | |
| | | | | | RHYNAWYGLGMIYYKQEKFSLAEMHFQKALDINP | |
| | | | | | QSSVLLCHIGVVQHALKKSEKALDTLNKAIVIDPKN | |
| | | | | | PLCKFHRASVLFANEKYKSALQELEELKQIVPKES | |
| | | | | | LVYFLIGKVYKKLGQTHLALMNFSWAMDLDPKGA | |
| | | | | | NNQIKEAIDKRYLPDDEEPITQEEQIMGTDESQES | |
| | | | | | SMTDADDTQLHAAESDEFNPAFLYKVVKASS | |
| 300 | JHU1 | NIV | 1.04193386 | CDC27 | MTVLQEPVQAAIWQALNHYAYRDAVFLAERLYAE | Core Human ORF Only |
| | 8346 | O_ | 3 | | VHSEEALFLLATCYYRSGKAYKAYRLLKGHSCTT | |
| | | RFS | | | PQCKYLLAKCCVDLSKLAEGEQILSGGVFNKQKS | |
| | | | | | HDDIVTEFGDSACFTLSLLGHVYCKTDRLAKGSE | |
| | | | | | CYQKSLSLNPFLWSPFESLCEIGEKPDPDQTFKF | |
| | | | | | TSLQNFSNCLPNSCTTQVPNHSLSHRQPETVLTE | |
| | | | | | TPQDTIELNRLNLESSNSKYSLNTDSSVSYIDSAVI | |
| | | | | | SPDTVPLGTGTSILSKQVQNKPKTGRSLLGGPAA | |
| | | | | | LSPLTPSFGILPLETPSPGDGSYLQNYTNTPPVID | |
| | | | | | VPSTGAPSKKTFRVLQSVARIGQTGTKSVFSQSG | |
| | | | | | NSREVTPILAQTQSSGPQTSTTPQVLSPTITSPPN | |
| | | | | | ALPRRSSRLFTSDSSTTKENSKKLKMKFPPEIPNRK | |
| | | | | | TKSKTNKGGITQPNINDSLEITKLDSSIISEGKIST | |
| | | | | | ITPQIQAFNLQKAAAEGLMSLLREMGKGYLALCSY | |
| | | | | | NCKEAINILSHLPSHHYNTGWVLCQIGRAYFELSE | |
| | | | | | YMQAERIFSEVRRIENYRVEGMEIYSTTLWHLQK | |
| | | | | | DVALSVLSKDLTDMDKNSPEAWCAAGNCFSLQR | |
| | | | | | EHDIAIKFFQRAIQVDPNYAYAYTLLGHEFVLTEEL | |
| | | | | | DKALACFRNAIRVNPRHYNAWYGLGMIYYKQEKF | |
| | | | | | SLAEMHFQKALDINPQSSVLLCHIGVVQHALKKSE | |
| | | | | | KALDTLNKAIVIDPKNPLCKFHRASVLFANEKYKS | |
| | | | | | ALQELEELKQIVPKESLVYFLIGKVYKKLGQTHLAL | |
| | | | | | MNFSWAMDLDPKGANNQIKEAIDKRYLPDDEEPI | |
| | | | | | TQEEQIMGTDESQESSMTDADDTQLHAAESDEF | |
| 301 | JHU1 | NIV | 0.43942407 | DEFB1 | MSPILGYWKIKGLVQPTRLLLEYLEEKYEEHLYER | Full Literal Sequence- |
| | 8839 | O_ | 9 | 29 | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKLLFPIFASLMLQY | |
| | | | | | QVNTEFIGLRRCLMGLGRCRDHCNVDEKEIQKCK | |
| | | | | | MKKCCVGPKVVKLIKNYLQYGTPNVLNEDVQEML | |
| | | | | | KPAKNSSAVIQRKHILSVLPQIKSTSFFANTNFVIIP | |
| | | | | | NATPMNSATISTMTPGQITYTATSTKSNTKESRDS | |
| | | | | | ATASPPPAPPPPNILPTPSLELEEAEEQ | |
| 302 | JHU1 | NIV | 0.43942407 | DEFB1 | MKLLFPIFASLMLQYQVNTEFIGLRRCLMGLGRC | Core Human ORF Only |
| | 8839 | O_ | 9 | 29 | RDHCNVDEKEIQKCKMKKCCVGPKVVKLIKNYLQ | |
| | | RFS | | | YGTPNVLNEDVQEMLKPAKNSSAVIQRKHILSVLP | |
| | | | | | QIKSTSFFANTNFVIIPNATPMNSATISTMTPGQIT | |
| | | | | | YTATSTKSNTKESRDSATASPPPAPPPPNILPTPS | |
| | | | | | LELEEAEEQ | |
| 303 | JHU1 | NIV | 2.48598948 | TBP | MSPILGYWKIKGLVQPTRLLLEYLEEKYEEHLYER | Full Literal Sequence- |
| | 9690 | O_ | 2 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDQNNSLPPYAQGL | |
| | | | | | ASPQGAMTPGIPIFSPMMPYGTGLTPQPIQNTNS | |
| | | | | | LSILEEQQRQQQQQQQQQQQQQQQQQQQQQQ | |
| | | | | | QQQQQQQQQQQQQQQAVAAAAVQQSTSQQAT | |
| | | | | | QGTSGQAPQLFHSQTLTTAPLPGTTPLYPSPMTP | |
| | | | | | MTPITPATPASESSGIVPQLQNIVSTVNLGCKLDL | |
| | | | | | KTIALRARNAEYNPKRFAAVIMRIREPRTTALIFSS | |
| | | | | | GKMVCTGAKSEEQSRLAARKYARVVQKLGFPAK | |
| | | | | | FLDFKIQNMVGSCDVKFPIRLEGLVLTHQQFSSYE | |
| | | | | | PELFPGLIYRMIKPRIVLLIFVSGKVVLTGAKVRA | |
| | | | | | EIYEAFENIYPILKGFRKTTNPAFLYKVVKASS | |
| 304 | JHU1 | NIV | 2.48598948 | TBP | MDQNNSLPPYAQGLASPQGAMTPGIPIFSPMMP | Core Human ORF Only |
| | 9690 | O_ | 2 | | YGTGLTPQPIQNTNSLSILEEQQRQQQQQQQQQ | |
| | | RFS | | | QQQQQQQQQQQQQQQQQQQQQQQQQQQQA | |
| | | | | | VAAAAVQQSTSQQATQGTSGQAPQLFHSQTLTT | |
| | | | | | APLPGTTPLYPSPMTPMTPITPATPASESSGIVPQ | |
| | | | | | LQNIVSTVNLGCKLDLKTIALRARNAEYNPKRFAA | |
| | | | | | VIMRIREPRTTALIFSSGKMVCTGAKSEEQSRLAA | |
| | | | | | RKYARVVQKLGFPAKFLDFKIQNMVGSCDVKFPIR | |
| | | | | | LEGLVLTHQQFSSYEPELFPGLIYRMIKPRIVLLIF | |
| | | | | | VSGKVVLTGAKVRAEIYEAFENIYPILKGFRKTT | |
| 305 | JHU2 | NIV | 1.58108267 | TAF6 | MSPILGYWKIKGLVQPTRLLLEYLEEKYEEHLYER | Full Literal Sequence- |
| | 2093 | O_ | 9 | | DEGDKWRNKKFELGLEFPNLPYYIDGDVKLTQSM | (including GST-tag, 6- |
| | | RFS | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAEEKKLKLSNTVLP | |
| | | | | | SESMKVVAESMGIAQIQEETCQLLTDEVSYRIKEI | |
| | | | | | AQDALKFMHMGKRQKLTTSDIDYALKLKNVEPLY | |
| | | | | | GFHAQEFIPFRFASGGGRELYFYEEKEVDLSDIIN | |
| | | | | | TPLPRVPLDVCLKAHWLSIEGCQPAIPENPPPAPK | |
| | | | | | EQQKAEATEPLKSAKPGQEEDGPLKGKGQGATT | |
| | | | | | ADGKGKEKKAPPLLEGAPLRLKPRSIHELSVEQQ | |
| | | | | | LYYKEITEACVGSCEAKRAEALQSIATDPGLYQML | |
| | | | | | PRFSTFISEGVRVNVVQNNLALLIYLMRMVKALMD | |
| | | | | | NPTLYLEKYVHELIPAVMTCIVSRQLCLRPDVDNH | |
| | | | | | WALRDFAARLVAQICKHFSTTTNNIQSRITKTFTK | |
| | | | | | SVWDEKTPWTTRYGSIAGLAELGHDVIKTLILPRL | |
| | | | | | QQEGERIRSVLDGPVLSNIDRIGADHVQSLLLKHC | |
| | | | | | APVLAKLRPPPDNQDAYRAEFGSLGPLLCSQVVK | |
| | | | | | ARAQAALQAQQVNRTTLTITQPRPTLTLSQAPQP | |
| | | | | | GPRTPGLLKVPGSIALPVQTLVSARAAAPPQPSP | |
| | | | | | PPTKFIVMSSSSSAPSTQQVLSLSTSAPGSGSTTT | |
| | | | | | SPVTTTVPSVQPIVKLVSTATTAPPSTAPSGPGSV | |
| | | | | | QKYIVVSLPPTGEGKGGPTSHPSPVPPPASSPSP | |
| | | | | | LSGSALCGGKQEAGDSPPPAPGTPKANGSQPNS | |
| | | | | | GSPQPAPNPAFLYKVVKASS | |
| 306 | JHU2 | NIV | 1.58108267 | TAF6 | MAEEKKLKLSNTVLPSESMKVVAESMGIAQIQEE | Core Human ORF Only |
| | 2093 | O_ | 9 | | TCQLLTDEVSYRIKEIAQDALKFMHMGKRQKLTTS | |
| | | RFS | | | DIDYALKLKNVEPLYGFHAQEFIPFRFASGGGREL | |
| | | | | | YFYEEKEVDLSDIINTPLPRVPLDVCLKAHWLSIEG | |
| | | | | | CQPAIPENPPPAPKEQQKAEATEPLKSAKPGQEE | |
| | | | | | DGPLKGKGQGATTADGKGKEKKAPPLLEGAPLR | |
| | | | | | LKPRSIHELSVEQQLYYKEITEACVGSCEAKRAEA | |
| | | | | | LQSIATDPGLYQMLPRFSTFISEGVRVNVVQNNLA | |
| | | | | | LLIYLMRMVKALMDNPTLYLEKYVHELIPAVMTCIV | |
| | | | | | SRQLCLRPDVDNHWALRDFAARLVAQICKHFSTT | |
| | | | | | TNNIQSRITKTFTKSVWDEKTPWTTRYGSIAGLAE | |
| | | | | | LGHDVIKTLILPRLQQEGERIRSVLDGPVLSNIDRI | |
| | | | | | GADHVQSLLLKHCAPVLAKLRPPPDNQDAYRAEF | |
| | | | | | GSLGPLLCSQVVKARAQAALQAQQVNRTTLTITQ | |
| | | | | | PRPTLTLSQAPQPGPRTPGLLKVPGSIALPVQTLV | |
| | | | | | SARAAAPPQPSPPPTKFIVMSSSSSAPSTQQVLS | |
| | | | | | LSTSAPGSGSTTTSPVTTTVPSVQPIVKLVSTATT | |
| | | | | | APPSTAPSGPGSVQKYIVVSLPPTGEGKGGPTSH | |
| | | | | | PSPVPPPASSPSPLSGSALCGGKQEAGDSPPPA | |
| | | | | | PGTPKANGSQPNSGSPQPAP | |
| 307 | JHUO | NIV | 0.07438717 | SLC30 | MSPILGYWKIKGLVQPTRLLLEYLEEKYEEHLYER | Full Literal Sequence- |
| | 0462 | O_T | 4 | A5 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEEKYGGDVLAGPG | |
| | | | | | GGGGLGPVDVPSARLTKYIVLLCFTKFLKAVGLFE | |
| | | | | | SYDLLKAVHIVQFIFILKLGTAFFMVLFQKPFSSGK | |
| | | | | | TITKHQIIGSLKIPGRKEFKDKKLNDPRKLVGN | |
| 308 | JHUO | NIV | 0.07438717 | SLC30 | MEEKYGGDVLAGPGGGGGLGPVDVPSARLTKYI | Core Human ORF Only |
| | 0462 | O_T | 4 | A5 | VLLCFTKFLKAVGLFESYDLLKAVHIVQFIFILKLGT | |
| | | OX | | | AFFMVLFQKPFSSGKTITKHQIIGSLKIPGRKEFKD | |
| | | | | | KKLNDPRKLVGN | |
| 309 | JHUO | NIV | 2.62573479 | ELMOD | MSPILGYWKIKGLVQPTRLLLEYLEEKYEEHLYER | Full Literal Sequence- |
| | 0937 | O_T | 2 | 3 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNEKSCSFHSKEEL | |
| | | | | | RDGQGERLSAGYSPSYDKDKSVLAFRGIPISELK | |
| | | | | | NHGILQALTTEAYEWEPRVVSTEVVRAQEEWEA | |
| | | | | | VDTIQPETGSQASSEQPGQLISFSEALQHFQTVD | |
| | | | | | LSPFKKRIQPTIRRTGLAALRHYLFGPPKLHQRLR | |
| | | | | | EERDLVLTIAQCGLDSQDPVHGRVLQTIYKKLTGS | |
| | | | | | KFDCALHGNHWEDLGFQGANPATDLRGAGFLAL | |
| | | | | | LHLLYLVMDSKTLPMAQEIFRLSRHHIQQFPFCLM | |
| | | | | | SVNITHIAIQALREECLSRECNRQQKVIPVVNSFYA | |
| | | | | | ATFLHLAHVWRTQRKTISDSGFVLKGVLFLLGRP | |
| | | | | | RLNAQCPRSREPKVVARLVLAAVLPHPHFLKFQL | |
| | | | | | TKISITHPLESASSPFSALTVALFWSYTYDKHIF | |
| 310 | JHUO | NIV | 2.62573479 | ELMOD | MNEKSCSFHSKEELRDGQGERLSAGYSPSYDKD | Core Human ORF Only |
| | 0937 | O_T | 2 | 3 | KSVLAFRGIPISELKNHGILQALTTEAYEWEPRVV | |
| | | OX | | | STEVVRAQEEWEAVDTIQPETGSQASSEQPGQLI | |
| | | | | | SFSEALQHFQTVDLSPFKKRIQPTIRRTGLAALRH | |
| | | | | | YLFGPPKLHQRLREERDLVLTIAQCGLDSQDPVH | |
| | | | | | GRVLQTIYKKLTGSKFDCALHGNHWEDLGFQGA | |
| | | | | | NPATDLRGAGFLALLHLLYLVMDSKTLPMAQEIFR | |
| | | | | | LSRHHIQQFPFCLMSVNITHIAIQALREECLSRECN | |
| | | | | | RQQKVIPVVNSFYAATFLHLAHVWRTQRKTISDS | |
| | | | | | GFVLKGVLFLLGRPRLNAQCPRSREPKVVARLVL | |
| | | | | | AAVLPHPHFLKFQLTKISITHPLESASSPFSALTVA | |
| | | | | | LFWSYTYDKHIF | |
| 311 | JHUO | NIV | 2.23449958 | SH3BP | MSPILGYWKIKGLVQPTRLLLEYLEEKYEEHLYER | Full Literal Sequence- |
| | 1616 | O_T | 5 | 5 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEQGLEEEEEVDPRI | |
| | | | | | QGELEKLNQSTDDINRRETELEDARQKFRSVLVE | |
| | | | | | ATVKLDELVKKIGKAVEDSKPYWEARRVARQAQL | |
| | | | | | EAQKATQDFQRATEVLRAAKETISLAEQRLLEDD | |
| | | | | | KRQFDSAWQEMLNHATQRVMEAEQTKTRSELV | |
| | | | | | HKETAARYNAAMGRMRQLEKKLKRAINKSKPYFE | |
| | | | | | LKAKYYVQLEQLKKTVDDLQAKLTLAKGEYKMAL | |
| | | | | | KNLEMISDEIHERRRSSAMGPRGCGVGAEGSST | |
| | | | | | SVEDLPGSKPEPDAISVASEAFEDDSCSNFVSED | |
| | | | | | DSETQSVSSFSSGPTSPSEMPDQFPAVVRPGSL | |
| | | | | | DLPSPVSLSEFGMMFPVLGPRSECSGASSPECE | |
| | | | | | VERGDRAEGAENKTSDKANNNRGLSSSSGSGG | |
| | | | | | SSKSQSSTSPEGQALENRMKQLSLQCSKGRDGII | |
| | | | | | ADIKMVQIG | |
| 312 | JHUO | NIV | 2.23449958 | SH3BP | MEQGLEEEEEVDPRIQGELEKLNQSTDDINRRET | Core Human ORF Only |
| | 1616 | O_T | 5 | 5 | ELEDARQKFRSVLVEATVKLDELVKKIGKAVEDSK | |
| | | OX | | | PYWEARRVARQAQLEAQKATQDFQRATEVLRAA | |
| | | | | | KETISLAEQRLLEDDKRQFDSAWQEMLNHATQR | |
| | | | | | VMEAEQTKTRSELVHKETAARYNAAMGRMRQLE | |
| | | | | | KKLKRAINKSKPYFELKAKYYVQLEQLKKTVDDLQ | |
| | | | | | AKLTLAKGEYKMALKNLEMISDEIHERRRSSAMG | |
| | | | | | PRGCGVGAEGSSTSVEDLPGSKPEPDAISVASEA | |
| | | | | | FEDDSCSNFVSEDDSETQSVSSFSSGPTSPSEM | |
| | | | | | PDQFPAVVRPGSLDLPSPVSLSEFGMMFPVLGP | |
| | | | | | RSECSGASSPECEVERGDRAEGAENKTSDKANN | |
| | | | | | NRGLSSSSGSGGSSKSQSSTSPEGQALENRMK | |
| | | | | | QLSLQCSKGRDGIIADIKMVQIG | |
| 313 | JHUO | NIV | 0.02236460 | ACRV1 | MSPILGYWKIKGLVQPTRLLLEYLEEKYEEHLYER | Full Literal Sequence- |
| | 1732 | O_T | 2 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNRFLLLMSLYLLGS | |
| | | | | | ARGTSSQPNELSGSIDHQTSVQQLPGEFFSLENP | |
| | | | | | SDAEALYETSSGLNTLSEHGSSEHGSSKHTVAEH | |
| | | | | | TSGEHAESEHASGEPAATEHAEGEHTVGEQPSG | |
| | | | | | EQPSGEHLSGEQPLSELESGEQPSDEQPSGEHG | |
| | | | | | SGEQPSGEQASGEQPSGEHASGEQASGAPISST | |
| | | | | | STGTILNCYTCAYMNDQGKCLRGEGTCITQNSQQ | |
| | | | | | CMLKKIFEGGKLQFMVQGCENMCPSMNLFSHGT | |
| | | | | | RMQIICCRNQSFCNKI | |
| 314 | JHUO | NIV | 0.02236460 | ACRV1 | MNRFLLLMSLYLLGSARGTSSQPNELSGSIDHQT | Core Human ORF Only |
| | 1732 | O_T | 2 | | SVQQLPGEFFSLENPSDAEALYETSSGLNTLSEH | |
| | | OX | | | GSSEHGSSKHTVAEHTSGEHAESEHASGEPAAT | |
| | | | | | EHAEGEHTVGEQPSGEQPSGEHLSGEQPLSELE | |
| | | | | | SGEQPSDEQPSGEHGSGEQPSGEQASGEQPSG | |
| | | | | | EHASGEQASGAPISSTSTGTILNCYTCAYMNDQG | |
| | | | | | KCLRGEGTCITQNSQQCMLKKIFEGGKLQFMVQ | |
| | | | | | GCENMCPSMNLFSHGTRMQIICCRNQSFCNKI | |
| 315 | JHUO | NIV | 0.55422596 | CD300 | MSPILGYWKIKGLVQPTRLLLEYLEEKYEEHLYER | Full Literal Sequence- |
| | 1936 | O_T | 1 | LB | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMCRRCKPELGQNFQ | |
| | | | | | SASGICICHWLQIRRTRSREGRAMWLPPALLLLSL | |
| | | | | | SGCFSIQGPESVRAPEQGSLTVQCHYKQGWETY | |
| | | | | | IKVWVCRGVRWDTCKILIETRGSEQGEKSDRVSIK | |
| | | | | | DNQKDRTFTVTMEGLRRDDADVYWCGIERRGPD | |
| | | | | | LGTQVKVIVDPEGAASTTASSPTNSNMAVFIGSH | |
| | | | | | KRNHYMLLVFVKVPILLILVTAILWLKGSQRVPEEP | |
| | | | | | GEQPIYMNFSEPLTKDMAT | |
| 316 | JHUO | NIV | 0.55422596 | CD300 | MCRRCKPELGQNFQSASGICICHWLQIRRTRSRE | Core Human ORF Only |
| | 1936 | O_T | 1 | LB | GRAMWLPPALLLLSLSGCFSIQGPESVRAPEQGS | |
| | | OX | | | LTVQCHYKQGWETYIKWWCRGVRWDTCKILIET | |
| | | | | | RGSEQGEKSDRVSIKDNQKDRTFTVTMEGLRRD | |
| | | | | | DADVYWCGIERRGPDLGTQVKVIVDPEGAASTTA | |
| | | | | | SSPTNSNMAVFIGSHKRNHYMLLVFVKVPILLILVT | |
| | | | | | AILWLKGSQRVPEEPGEQPIYMNFSEPLTKDMAT | |
| 317 | JHUO | NIV | 0.41129088 | PSMA4 | MSPILGYWKIKGLVQPTRLLLEYLEEKYEEHLYER | Full Literal Sequence- |
| | 1993 | O_T | 2 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSRRYDSRTTIFSPE | |
| | | | | | GRLYQVEYAMEAIGHAGTCLGILANDGVLLAAER | |
| | | | | | RNIHKLLDEVFFSEKIYKLNEDMACSVAGITSDAN | |
| | | | | | VLTNELRLIAQRYLLQYQEPIPCEQLVTALCDIKQA | |
| | | | | | YTQFGGKRPFGVSLLYIGWDKHYGFQLYQSDPS | |
| | | | | | GNYGGWKATCIGNNSAAAVSMLKQDYKEGEMTL | |
| | | | | | KSALALAIKVLNKTMDVSKLSAEKVEIATLTRENG | |
| | | | | | KTVIRVLKQKEVEQLIKKHEEEEAKAEREKKEKEQ | |
| | | | | | KEKDK | |
| 318 | JHUO | NIV | 0.41129088 | PSMA4 | MSRRYDSRTTIFSPEGRLYQVEYAMEAIGHAGTC | Core Human ORF Only |
| | 1993 | O_T | 2 | | LGILANDGVLLAAERRNIHKLLDEVFFSEKIYKLNE | |
| | | OX | | | DMACSVAGITSDANVLTNELRLIAQRYLLQYQEPI | |
| | | | | | PCEQLVTALCDIKQAYTQFGGKRPFGVSLLYIGW | |
| | | | | | DKHYGFQLYQSDPSGNYGGWKATCIGNNSAAAV | |
| | | | | | SMLKQDYKEGEMTLKSALALAIKVLNKTMDVSKL | |
| | | | | | SAEKVEIATLTRENGKTVIRVLKQKEVEQLIKKHEE | |
| | | | | | EEAKAEREKKEKEQKEKDK | |
| 319 | JHUO | NIV | 0.74926177 | BCAS3 | MSPILGYWKIKGLVQPTRLLLEYLEEKYEEHLYER | Full Literal Sequence- |
| | 2507 | O_T | 3 | _frag | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVVMPLAQIKQPMTL | |
| | | | | | GTITKRTGKVKPPPQISPSKSMGGEFCVAAIFGTS | |
| | | | | | RSWFANNAGLKREKDQSKQVVVESLYIISCYGTL | |
| | | | | | VEHMMEPRPLSTAPKISDDTPLEMMTSPRASWTL | |
| | | | | | VRTPQWNELQPPFNANHPLLLAADAVQYYQFLLA | |
| | | | | | GLVPPGSPGPITRHGSYDSLASDHSGQEDEEWL | |
| | | | | | SQVEIVTHTGPHRRLWMGPQFQFKTIHPSGQTTV | |
| | | | | | ISSSSSVLQSHGPSDTPQPLLDFDTDDLDLNSLRI | |
| | | | | | QPVRSDPVSMPGSSRPVSDRRGVSTVIDAASGT | |
| | | | | | FDRSVTLLEVCGSWPEGFGLRHMSSMEHTEEGL | |
| | | | | | RERLADAMAESPSRDVVGSGTELQREGSIETLSN | |
| | | | | | SSGSTSGSIPRNFDGYRSPLPTNESQPLSLFPTG | |
| | | | | | FP | |
| 320 | JHUO | NIV | 0.74926177 | BCAS3 | MVVMPLAQIKQPMTLGTITKRTGKVKPPPQISPSK | Core Human ORF Only |
| | 2507 | O_T | 3 | _frag | SMGGEFCVAAIFGTSRSWFANNAGLKREKDQSK | |
| | | OX | | | QVVVESLYIISCYGTLVEHMMEPRPLSTAPKISDD | |
| | | | | | TPLEMMTSPRASWTLVRTPQWNELQPPFNANHP | |
| | | | | | LLLAADAVQYYQFLLAGLVPPGSPGPITRHGSYD | |
| | | | | | SLASDHSGQEDEEWLSQVEIVTHTGPHRRLWMG | |
| | | | | | PQFQFKTIHPSGQTTVISSSSSVLQSHGPSDTPQ | |
| | | | | | PLLDFDTDDLDLNSLRIQPVRSDPVSMPGSSRPV | |
| | | | | | SDRRGVSTVIDAASGTFDRSVTLLEVCGSWPEGF | |
| | | | | | GLRHMSSMEHTEEGLRERLADAMAESPSRDVVG | |
| | | | | | SGTELQREGSIETLSNSSGSTSGSIPRNFDGYRS | |
| | | | | | PLPTNESQPLSLFPTGFP | |
| 321 | JHUO | NIV | 1.00912497 | HAAO | MSPILGYWKIKGLVQPTRLLLEYLEEKYEEHLYER | Full Literal Sequence- |
| | 3691 | O_T | 5 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMERRLGVRAVWKEN | |
| | | | | | RGSFQPPVCNKLMHQEQLKVMFIGGPNTRKDYHI | |
| | | | | | EEGEEVFYQLEGDMVLRVLEQGKHRDVVIRQGEI | |
| | | | | | FLLPARVPHSPQRFANTVGLVVERRRLETELDGL | |
| | | | | | RYYVGDTMDVLFEKWFYCKDLGTQLAPIIQEFFS | |
| | | | | | SEQYRTGKPIPDQLLKEPPFPLSTRSIMEPMSLDA | |
| | | | | | WLDSHHRELQAGTPLSLFGDTYETQVIAYGQGSS | |
| | | | | | EGLRQNVDVWLWQLEGSSVVTMGGRRLSLAPD | |
| | | | | | DSLLVLAGTSYAWERTQGSVALSVTQDPACKKPL | |
| | | | | | G | |
| 322 | JHUO | NIV | 1.00912497 | HAAO | MERRLGVRAWVKENRGSFQPPVCNKLMHQEQL | Core Human ORF Only |
| | 3691 | O_T | 5 | | KVMFIGGPNTRKDYHIEEGEEVFYQLEGDMVLRV | |
| | | OX | | | LEQGKHRDVVIRQGEIFLLPARVPHSPQRFANTV | |
| | | | | | GLWERRRLETELDGLRYYVGDTMDVLFEKWFY | |
| | | | | | CKDLGTQLAPIIQEFFSSEQYRTGKPIPDQLLKEP | |
| | | | | | PFPLSTRSIMEPMSLDAWLDSHHRELQAGTPLSL | |
| | | | | | FGDTYETQVIAYGQGSSEGLRQNVDVWLWQLEG | |
| | | | | | SSVVTMGGRRLSLAPDDSLLVLAGTSYAWERTQ | |
| | | | | | GSVALSVTQDPACKKPLG | |
| 323 | JHUO | NIV | 0.89327015 | CHIC2 | MSPILGYWKIKGLVQPTRLLLEYLEEKYEEHLYER | Full Literal Sequence- |
| | 3864 | O_T | 4 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMADFDEIYEEEEDEE | |
| | | | | | RALEEQLLKYSPDPVVVRGSGHVTVFGLSNKFES | |
| | | | | | EFPSSLTGKVAPEEFKASINRVNSCLKKNLPVNVR | |
| | | | | | WLLCGCLCCCCTLGCSMWPVICLSKRTRRSIEKL | |
| | | | | | LEWENNRLYHKLCLHWRLSKRKCETNNMMEYVI | |
| | | | | | LIEFLPKTPIFRPD | |
| 324 | JHUO | NIV | 0.89327015 | CHIC2 | MADFDEIYEEEEDEERALEEQLLKYSPDPVVVRG | Core Human ORF Only |
| | 3864 | O_T | 4 | | SGHVTVFGLSNKFESEFPSSLTGKVAPEEFKASIN | |
| | | OX | | | RVNSCLKKNLPVNVRWLLCGCLCCCCTLGCSMW | |
| | | | | | PVICLSKRTRRSIEKLLEWENNRLYHKLCLHWRLS | |
| | | | | | KRKCETNNMMEYVILIEFLPKTPIFRPD | |
| 325 | JHUO | NIV | 1.58952041 | CHIC2 | MSPILGYWKIKGLVQPTRLLLEYLEEKYEEHLYER | Full Literal Sequence- |
| | 3864 | O_T | 5 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMADFDEIYEEEEDEE | |
| | | | | | RALEEQLLKYSPDPVVVRGSGHVTVFGLSNKFES | |
| | | | | | EFPSSLTGKVAPEEFKASINRVNSCLKKNLPVNVR | |
| | | | | | WLLCGCLCCCCTLGCSMWPVICLSKRTRRSIEKL | |
| | | | | | LEWENNRLYHKLCLHWRLSKRKCETNNMMEYVI | |
| | | | | | LIEFLPKTPIFRPD | |
| 326 | JHUO | NIV | 1.58952041 | CHIC2 | MADFDEIYEEEEDEERALEEQLLKYSPDPVVVRG | Core Human ORF Only |
| | 3864 | O_T | 5 | | SGHVTVFGLSNKFESEFPSSLTGKVAPEEFKASIN | |
| | | OX | | | RVNSCLKKNLPVNVRWLLCGCLCCCCTLGCSMW | |
| | | | | | PVICLSKRTRRSIEKLLEWENNRLYHKLCLHWRLS | |
| | | | | | KRKCETNNMMEYVILIEFLPKTPIFRPD | |
| 327 | JHUO | NIV | 0.42275417 | TMEM3 | MSPILGYWKIKGLVQPTRLLLEYLEEKYEEHLYER | Full Literal Sequence- |
| | 3929 | O_T | 8 | 1 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMRLTEKSEGEQQLK | |
| | | | | | PNNSNAPNEDQEEEIQQSEQHTPARQRTQRADT | |
| | | | | | QPSRCRLPSRRTPTTSSDRTINLLEVLPWPTEWIF | |
| | | | | | NPYRLPALFELYPEFLLVFKEAFHDISHCLKAQME | |
| | | | | | KIGLPIILHLFALSTLYFYKFFLPTILSLSFFILL | |
| | | | | | VLLLFIIVFILIFF | |
| 328 | JHUO | NIV | 0.42275417 | TMEM3 | MRLTEKSEGEQQLKPNNSNAPNEDQEEEIQQSE | Core Human ORF Only |
| | 3929 | O_T | 8 | 1 | QHTPARQRTQRADTQPSRCRLPSRRTPTTSSDR | |
| | | OX | | | TINLLEVLPWPTEWIFNPYRLPALFELYPEFLLVFK | |
| | | | | | EAFHDISHCLKAQMEKIGLPIILHLFALSTLYFYKFF | |
| | | | | | LPTILSLSFFILLVLLLFIIVFILIFF | |
| 329 | JHUO | NIV | 0.24938664 | EEF1G | MSPILGYWKIKGLVQPTRLLLEYLEEKYEEHLYER | Full Literal Sequence- |
| | 4724 | O_T | 3 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAGTLYTYPENWR | |
| | | | | | AFKALIAAQYSGAQVRVLSAPPHFHFGQTNRTPE | |
| | | | | | FLRKFPAGKVPAFEGDDGFCVFESNAIAYYVSNE | |
| | | | | | ELRGSTPEAAAQVVQVWSFADSDIVPPASTVWF | |
| | | | | | PTLGIMHHNKQATENAKEEVRRILGLLDAYLKTRT | |
| | | | | | FLVGERVTLADITVVCTLLWLYKQVLEPSFRQAFP | |
| | | | | | NTNRWFLTCINQPQFRAVLGEVKLCEKMAQFDA | |
| | | | | | KKFAETQPKKDTPRKEKGSREEKQKPQAERKEE | |
| | | | | | KKAAAPAPEEEMDECEQALAAEPKAKDPFAHLPK | |
| | | | | | STFVLDEFKRKYSNEDTLSVALPYFWEHFDKDG | |
| | | | | | WSLWYSEYRFPEELTQTFMSCNLITGMFQRLDKL | |
| | | | | | RKNAFASVILFGTNNSSSISGVVWFRGQELAFPLS | |
| | | | | | PDWQVDYESYTWRKLDPGSEETQTLVREYFSW | |
| | | | | | EGAFQHVGKAFNQGKIFK | |
| 330 | JHUO | NIV | 0.24938664 | EEF1G | MAAGTLYTYPENWRAFKALIAAQYSGAQVRVLSA | Core Human ORF Only |
| | 4724 | O_T | 3 | | PPHFHFGQTNRTPEFLRKFPAGKVPAFEGDDGF | |
| | | OX | | | CVFESNAIAYYVSNEELRGSTPEAAAQVVQVWSF | |
| | | | | | ADSDIVPPASTVWFPTLGIMHHNKQATENAKEEV | |
| | | | | | RRILGLLDAYLKTRTFLVGERVTLADITVVCTLLWL | |
| | | | | | YKQVLEPSFRQAFPNTNRWFLTCINQPQFRAVLG | |
| | | | | | EVKLCEKMAQFDAKKFAETQPKKDTPRKEKGSR | |
| | | | | | EEKQKPQAERKEEKKAAAPAPEEEMDECEQALA | |
| | | | | | AEPKAKDPFAHLPKSTFVLDEFKRKYSNEDTLSV | |
| | | | | | ALPYFWEHFDKDGWSLWYSEYRFPEELTQTFMS | |
| | | | | | CNLITGMFQRLDKLRKNAFASVILFGTNNSSSISG | |
| | | | | | VVWFRGQELAFPLSPDWQVDYESYTWRKLDPG | |
| | | | | | SEETQTLVREYFSWEGAFQHVGKAFNQGKIFK | |
| 331 | JHUO | NIV | 0.82515646 | METTL | MSPILGYWKIKGLVQPTRLLLEYLEEKYEEHLYER | Full Literal Sequence- |
| | 4842 | O_T | 8 | 13 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNLLPKSSREFGSV | |
| | | | | | DYWEKFFQQRGKKAFEWYGTYLELCGVLHKYIK | |
| | | | | | PREKVLVIGCGNSELSEQLYDVGYRDIVNIDISEV | |
| | | | | | VIKQMKECNATRRPQMSFLKMDMTQMEFPDASF | |
| | | | | | QVVLDKGTLDAVLTDEEEKTLQQVDRMLAEVGR | |
| | | | | | VLQVGGRYLCISLAQAHILKKAVGHFSREGWMVR | |
| | | | | | VHQVANSQDQVLEAEPQFSLPVFAFIMTKFRPVP | |
| | | | | | GSALQIFELCAQEQRKPVRLESAERLAEAVQERQ | |
| | | | | | QYAWLCSQLRRKARLGSVSLDLCDGDTGEPRYT | |
| | | | | | LHVVDSPTVKPSRDNHFAIFIIPQGRETEWLFGMD | |
| | | | | | EGRKQLAASAGFRRLITVALHRGQQYESMDHIQA | |
| | | | | | ELSARVMELAPAGMPTQQQVPFLSVGGDIGVRT | |
| | | | | | VQHQDCSPLSGDYVIEDVQGDDKRYFRRLIFLSN | |
| | | | | | RNVVQSEARLLKDVSHKAQKKRKKDRKKQRPAD | |
| | | | | | AEDLPAAPGQSIDKSYLCCEHHKAMIAGLALLRNP | |
| | | | | | ELLLEIPLALLVVGLGGGSLPLFVHDHFPKSCIDAV | |
| | | | | | EIDPSMLEVATQWFGFSQSDRMKVHIADGLDYIA | |
| | | | | | SLAGGGEARPCYDVIMFDVDSKDPTLGMSCPPP | |
| | | | | | AFVEQSFLQKVKSILTPEGVFILNLVCRDLGLKDS | |
| | | | | | VLAGLKAVFPLLYVRRIEGEVNEILFCQLHPEQKL | |
| | | | | | ATPELLETAQALERTLRKPGRGWDDTYVLSDMLK | |
| | | | | | TVKIV | |
| 332 | JHUO | NIV | 0.82515646 | METTL | MNLLPKSSREFGSVDYWEKFFQQRGKKAFEWY | Core Human ORF Only |
| | 4842 | O_T | 8 | 13 | GTYLELCGVLHKYIKPREKVLVIGCGNSELSEQLY | |
| | | OX | | | DVGYRDIVNIDISEVVIKQMKECNATRRPQMSFLK | |
| | | | | | MDMTQMEFPDASFQVVLDKGTLDAVLTDEEEKT | |
| | | | | | LQQVDRMLAEVGRVLQVGGRYLCISLAQAHILKK | |
| | | | | | AVGHFSREGWMVRVHQVANSQDQVLEAEPQFS | |
| | | | | | LPVFAFIMTKFRPVPGSALQIFELCAQEQRKPVRL | |
| | | | | | ESAERLAEAVQERQQYAWLCSQLRRKARLGSVS | |
| | | | | | LDLCDGDTGEPRYTLHVVDSPTVKPSRDNHFAIFI | |
| | | | | | IPQGRETEWLFGMDEGRKQLAASAGFRRLITVAL | |
| | | | | | HRGQQYESMDHIQAELSARVMELAPAGMPTQQ | |
| | | | | | QVPFLSVGGDIGVRTVQHQDCSPLSGDYVIEDVQ | |
| | | | | | GDDKRYFRRLIFLSNRNVVQSEARLLKDVSHKAQ | |
| | | | | | KKRKKDRKKQRPADAEDLPAAPGQSIDKSYLCCE | |
| | | | | | HHKAMIAGLALLRNPELLLEIPLALLVVGLGGGSLP | |
| | | | | | LFVHDHFPKSCIDAVEIDPSMLEVATQWFGFSQS | |
| | | | | | DRMKVHIADGLDYIASLAGGGEARPCYDVIMFDV | |
| | | | | | DSKDPTLGMSCPPPAFVEQSFLQKVKSILTPEGV | |
| | | | | | FILNLVCRDLGLKDSVLAGLKAVFPLLYVRRIEGEV | |
| | | | | | NEILFCQLHPEQKLATPELLETAQALERTLRKPGR | |
| | | | | | GWDDTYVLSDMLKTVKIV | |
| 333 | JHUO | NIV | 0.71017528 | SFXN1 | MSPILGYWKIKGLVQPTRLLLEYLEEKYEEHLYER | Full Literal Sequence- |
| | 4872 | O_T | 3 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMILIGRMSAQVPMNM | |
| | | | | | TITGCMMTFYRTTPAVLFWQWINQSFNAVVNYTN | |
| | | | | | RSGDAPLTVNELGTAYVSATTGAVATALGLNALT | |
| | | | | | KHVSPLIGRFVPFAAVAAANCINIPLMRQRELKVGI | |
| | | | | | PVTDENGNRLGESANAAKQAITQVVVSRILMAAP | |
| | | | | | GMAIPPFIMNTLEKKAFLKRFPWMSAPIQVGLVGF | |
| | | | | | CLVFATPLCCALFPQKSSMSVTSLEAELQAKIQES | |
| | | | | | HPELRRVYFNKGL | |
| 334 | JHUO | NIV | 0.71017528 | SFXN1 | MILIGRMSAQVPMNMTITGCMMTFYRTTPAVLFW | Core Human ORF Only |
| | 4872 | O_T | 3 | | QWINQSFNAVVNYTNRSGDAPLTVNELGTAYVSA | |
| | | OX | | | TTGAVATALGLNALTKHVSPLIGRFVPFAAVAAAN | |
| | | | | | CINIPLMRQRELKVGIPVTDENGNRLGESANAAK | |
| | | | | | QAITQVVVSRILMAAPGMAIPPFIMNTLEKKAFLKR | |
| | | | | | FPWMSAPIQVGLVGFCLVFATPLCCALFPQKSSM | |
| | | | | | SVTSLEAELQAKIQESHPELRRVYFNKGL | |
| 335 | JHUO | NIV | 0.07946444 | HSD3B | MSPILGYWKIKGLVQPTRLLLEYLEEKYEEHLYER | Full Literal Sequence- |
| | 5610 | O_T | 9 | 1 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTGWSCLVTGAGGF | |
| | | | | | LGQRIIRLLVKEKELKEIRVLDKAFGPELREEFSKL | |
| | | | | | QNKTKLTVLEGDILDEPFLKRACQDVSVIIHTACIID | |
| | | | | | VFGVTHRESIMNVNVKGTQLLLEACVQASVPVFIY | |
| | | | | | TSSIEVAGPNSYKEIIQNGHEEEPLENTWPAPYPH | |
| | | | | | SKKLAEKAVLAANGWNLKNGGTLYTCALRPMYIY | |
| | | | | | GEGSRFLSASINEALNNNGILSSVGKFSTVNPVYV | |
| | | | | | GNVAWAHILALRALQDPKKAPSIRGQFYYISDDTP | |
| | | | | | HQSYDNLNYTLSKEFGLRLDSRWSFPLSLMYWIG | |
| | | | | | FLLEIVSFLLRPIYTYRPPFNRHIVTLSNSVFTFSYK | |
| | | | | | KAQRDLAYKPLYSWEEAKQKTVEVWGSLVDRHK | |
| | | | | | ENLKSKTQ | |
| 336 | JHUO | NIV | 0.07946444 | HSD3B | MTGWSCLVTGAGGFLGQRIIRLLVKEKELKEIRVL | Core Human ORF Only |
| | 5610 | O_T | 9 | 1 | DKAFGPELREEFSKLQNKTKLTVLEGDILDEPFLK | |
| | | OX | | | RACQDVSVIIHTACIIDVFGVTHRESIMNVNVKGT | |
| | | | | | QLLLEACVQASVPVFIYTSSIEVAGPNSYKEIIQNG | |
| | | | | | HEEEPLENTWPAPYPHSKKLAEKAVLAANGWNL | |
| | | | | | KNGGTLYTCALRPMYIYGEGSRFLSASINEALNN | |
| | | | | | NGILSSVGKFSTVNPVYVGNVAWAHILALRALQD | |
| | | | | | PKKAPSIRGQFYYISDDTPHQSYDNLNYTLSKEFG | |
| | | | | | LRLDSRWSFPLSLMYWIGFLLEIVSFLLRPIYTYRP | |
| | | | | | PFNRHIVTLSNSVFTFSYKKAQRDLAYKPLYSWE | |
| | | | | | EAKQKTVEWVGSLVDRHKENLKSKTQ | |
| 337 | JHUO | NIV | 0.60162968 | TMEM1 | MSPILGYWKIKGLVQPTRLLLEYLEEKYEEHLYER | Full Literal Sequence- |
| | 5937 | O_T | 5 | 06B | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGKSLSHLPLHSSKE | |
| | | | | | DAYDGVTSENMRNGLVNSEVHNEDGRNGDVSQ | |
| | | | | | FPYVEFTGRDSVTCPTCQGTGRIPRGQENQLVAL | |
| | | | | | IPYSDQRLRPRRTKLYVMASVFVCLLLSGLAVFFL | |
| | | | | | FPRSIDVKYIGVKSAYVSYDVQKRTIYLNITNTLNIT | |
| | | | | | NNNYYSVEVENITAQVQFSKTVIGKARLNNISIIGP | |
| | | | | | LDMKQIDYTVPTVIAEEMSYMYDFCTLISIKVHNIV | |
| | | | | | LMMQVTVTTTYFGHSEQISQERYQYVDCGRNTT | |
| | | | | | YQLGQSEYLNVLQPQQ | |
| 338 | JHUO | NIV | 0.60162968 | TMEM1 | MGKSLSHLPLHSSKEDAYDGVTSENMRNGLVNS | Core Human ORF Only |
| | 5937 | O_T | 5 | 06B | EVHNEDGRNGDVSQFPYVEFTGRDSVTCPTCQG | |
| | | OX | | | TGRIPRGQENQLVALIPYSDQRLRPRRTKLYVMA | |
| | | | | | SVFVCLLLSGLAVFFLFPRSIDVKYIGVKSAYVSYD | |
| | | | | | VQKRTIYLNITNTLNITNNNYYSVEVENITAQVQFS | |
| | | | | | KTVIGKARLNNISIIGPLDMKQIDYTVPTVIAEEMSY | |
| | | | | | MYDFCTLISIKVHNIVLMMQVTVTTTYFGHSEQIS | |
| | | | | | QERYQYVDCGRNTTYQLGQSEYLNVLQPQQ | |
| 339 | JHUO | NIV | 1.34431443 | CAPS2 | MSPILGYWKIKGLVQPTRLLLEYLEEKYEEHLYER | Full Literal Sequence- |
| | 6537 | O_T | 9 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMIDHLSRAVISDPEQ | |
| | | | | | NLAIEQKESDHILPDSKMTPLRFRKRTLHETKIRTH | |
| | | | | | STLTENVLSHKLQFDGRIVSRTNVLPFIQKSIYSHQ | |
| | | | | | CGRRKGKQYRLGDFYVGATLTFLSSDHLSLPESI | |
| | | | | | KENTLLKLRITNIDQIALDSLKTASMEQEDDIIIQET | |
| | | | | | NDRLVFKAIQDVLKEKLHKRGVRILTGLGKYFQQL | |
| | | | | | DKEGNGLLDKADFKQALKVFHLEVSEKDFESAWL | |
| | | | | | ILNDNGNGKVDYGEFKRGIIGEMNEYRKSYVRKA | |
| | | | | | FMKLDFNKSGSVPIINIRKCYCAKKHSQVISG | |
| 340 | JHUO | NIV | 1.34431443 | CAPS2 | MIDHLSRAVISDPEQNLAIEQKESDHILPDSKMTPL | Core Human ORF Only |
| | 6537 | O_T | 9 | | RFRKRTLHETKIRTHSTLTENVLSHKLQFDGRIVS | |
| | | OX | | | RTNVLPFIQKSIYSHQCGRRKGKQYRLGDFYVGA | |
| | | | | | TLTFLSSDHLSLPESIKENTLLKLRITNIDQIALDSL | |
| | | | | | KTASMEQEDDIIIQETNDRLVFKAIQDVLKEKLHKR | |
| | | | | | GVRILTGLGKYFQQLDKEGNGLLDKADFKQALKV | |
| | | | | | FHLEVSEKDFESAWLILNDNGNGKVDYGEFKRGII | |
| | | | | | GEMNEYRKSYVRKAFMKLDFNKSGSVPIINIRKCY | |
| | | | | | CAKKHSQVISG | |
| 341 | JHUO | NIV | 0.19776220 | ABHD1 | MSPILGYWKIKGLVQPTRLLLEYLEEKYEEHLYER | Full Literal Sequence- |
| | 6624 | O_T | 3 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLSSFLSPQNGTWA | |
| | | | | | DTFSLLLALAVALYLGYYWACVLQRPRLVAGPQF | |
| | | | | | LAFLEPHCSITTETFYPTLWCFEGRLQSIFQVLLQ | |
| | | | | | SQPLVLYQSDILQTPDGGQLLLDWAKQPDSSQD | |
| | | | | | PDPTTQPIVLLLPGITGSSQETYVLHLVNQALRDG | |
| | | | | | YQAVVFNNRGCRGEELRTHRAFCASNTEDLETV | |
| | | | | | VNHIKHRYPQAPLLAVGISFGGILVLNHLAQARQA | |
| | | | | | AGLVAALTLSACWDSFETTRSLETPLNSLLFNQPL | |
| | | | | | TAGLCQLVERNRKVIEKVVDIDFVLQARTIRQFDE | |
| | | | | | RYTSVAFGYQDCVTYYKAASPRTKIDAIRIPVLYLS | |
| | | | | | AADDPFSPVCALPIQAAQHSPYVALLITARGGHIG | |
| | | | | | FLEGLLPWQHWYMSRLLHQYAKAIFQDPEGLPDL | |
| | | | | | RALLPSEDRNS | |
| 342 | JHUO | NIV | 0.19776220 | ABHD1 | MLSSFLSPQNGTWADTFSLLLALAVALYLGYYWA | Core Human ORF Only |
| | 6624 | O_T | 3 | | CVLQRPRLVAGPQFLAFLEPHCSII IEIYPTLWC | |
| | | OX | | | FEGRLQSIFQVLLQSQPLVLYQSDILQTPDGGQLL | |
| | | | | | LDWAKQPDSSQDPDPTTQPIVLLLPGITGSSQET | |
| | | | | | YVLHLVNQALRDGYQAVVFNNRGCRGEELRTHR | |
| | | | | | AFCASNTEDLETVVNHIKHRYPQAPLLAVGISFGG | |
| | | | | | ILVLNHLAQARQAAGLVAALTLSACWDSFETTRSL | |
| | | | | | ETPLNSLLFNQPLTAGLCQLVERNRKVIEKVVDID | |
| | | | | | FVLQARTIRQFDERYTSVAFGYQDCVTYYKAASP | |
| | | | | | RTKIDAIRIPVLYLSAADDPFSPVCALPIQAAQHSP | |
| | | | | | YVALLITARGGHIGFLEGLLPWQHWYMSRLLHQY | |
| | | | | | AKAIFQDPEGLPDLRALLPSEDRNS | |
| 343 | JHUO | NIV | 0.90535382 | OGFR | MSPILGYWKIKGLVQPTRLLLEYLEEKYEEHLYER | Full Literal Sequence- |
| | 8317 | O_T | 8 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDDPDCDSTWEEDE | |
| | | | | | EDAEDAEDEDCEDGEAAGARDADAGDEDEESEE | |
| | | | | | PRAARPSSFQSRMTGSRNWRATRDMCRYRHNY | |
| | | | | | PDLVERDCNGDTPNLSFYRNEIRFLPNGCFIEDIL | |
| | | | | | QNWTDNYDLLEDNHSYIQWLFPLREPGVNWHAK | |
| | | | | | PLTLREVEVFKSSQEIQERLVRAYELMLGFYGIRL | |
| | | | | | EDRGTGTVGRAQNYQKRFQNLNWRSHNNLRITR | |
| | | | | | ILKSLGELGLEHFQAPLVRFFLEETLVRRELPGVR | |
| | | | | | QSALDYFMFAVRCRHQRRQLVHFAWEHFRPRC | |
| | | | | | KFVWGPQDKLRRFKPSSLPHPLEGSRKVEEEGS | |
| | | | | | PGDPDHEASTQGRTCGPEHSKGGGRVDEGPQP | |
| | | | | | RSVEPQDAGPLERSQGDEAGGHGEDRPEPLSPK | |
| | | | | | ESKKRKLELSRREQPPTEPGPQSASEVEKIALNL | |
| | | | | | EGCALSQGSLRTGTQEVGGQDPGEAVQPCRQP | |
| | | | | | LGARVADKVRKRRKVDEGAGDSAAVASGGAQTL | |
| | | | | | ALAGSPAPSGHPKAGHSENGVEEDTEGRTGPKE | |
| | | | | | GTPGSPSETPGPSPAGPAGDEPAESPSETPGPR | |
| | | | | | PAGPAGDEPAESPSETPGPSPAGPTRDEPAESP | |
| | | | | | SETPGPRPAGPAGDEPAESPSETPGPRPAGPAG | |
| | | | | | DEPAESPSETPGPSPAGPTRDEPAKAGEAAELQ | |
| | | | | | DAEVESSAKSGKP | |
| 344 | JHUO | NIV | 0.90535382 | OGFR | MDDPDCDSTWEEDEEDAEDAEDEDCEDGEAAG | Core Human ORF Only |
| | 8317 | O_T | 8 | | ARDADAGDEDEESEEPRAARPSSFQSRMTGSRN | |
| | | OX | | | WRATRDMCRYRHNYPDLVERDCNGDTPNLSFY | |
| | | | | | RNEIRFLPNGCFIEDILQNWTDNYDLLEDNHSYIQ | |
| | | | | | WLFPLREPGVNWHAKPLTLREVEVFKSSQEIQER | |
| | | | | | LVRAYELMLGFYGIRLEDRGTGTVGRAQNYQKRF | |
| | | | | | QNLNWRSHNNLRITRILKSLGELGLEHFQAPLVRF | |
| | | | | | FLEETLVRRELPGVRQSALDYFMFAVRCRHQRR | |
| | | | | | QLVHFAWEHFRPRCKFVWGPQDKLRRFKPSSLP | |
| | | | | | HPLEGSRKVEEEGSPGDPDHEASTQGRTCGPEH | |
| | | | | | SKGGGRVDEGPQPRSVEPQDAGPLERSQGDEA | |
| | | | | | GGHGEDRPEPLSPKESKKRKLELSRREQPPTEP | |
| | | | | | GPQSASEVEKIALNLEGCALSQGSLRTGTQEVGG | |
| | | | | | QDPGEAVQPCRQPLGARVADKVRKRRKVDEGA | |
| | | | | | GDSAAVASGGAQTLALAGSPAPSGHPKAGHSEN | |
| | | | | | GVEEDTEGRTGPKEGTPGSPSETPGPSPAGPAG | |
| | | | | | DEPAESPSETPGPRPAGPAGDEPAESPSETPGP | |
| | | | | | SPAGPTRDEPAESPSETPGPRPAGPAGDEPAES | |
| | | | | | PSETPGPRPAGPAGDEPAESPSETPGPSPAGPT | |
| | | | | | RDEPAKAGEAAELQDAEVESSAKSGKP | |
| 345 | JHUO | NIV | 2.86648284 | EPB41 | MSPILGYWKIKGLVQPTRLLLEYLEEKYEEHLYER | Full Literal Sequence- |
| | 8576 | O_T | 5 | L1 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEEKDYSEADGLSE | |
| | | | | | RTTPSKAQKSPQKIAKKYKSAICRVTLLDASEYEC | |
| | | | | | EVEKHGRGQVLFDLVCEHLNLLEKDYFGLTFCDA | |
| | | | | | DSQKNWLDPSKEIKKQIRSSPWNFAFTVKFYPPD | |
| | | | | | PAQLTEDITRYYLCLQLRADIITGRLPCSFVTHALL | |
| | | | | | GSYAVQAELGDYDAEEHVGNYVSELRFAPNQTR | |
| | | | | | ELEERIMELHKTYRGMTPGEAEIHFLENAKKLSMY | |
| | | | | | GVDLHHAKDSEGIDIMLGVCANGLLIYRDRLRINR | |
| | | | | | FAWPKILKISYKRSNFYIKIRPGEYEQFESTIGFKL | |
| | | | | | PNHRSAKRLWKVCIEHHTFFRLVSPEPPPKGFLV | |
| | | | | | MGSKFRYSGRTQAQTRQASALIDRPAPFFERSSS | |
| | | | | | KRYTMSRSLDGAEFSRPASVSENHDAGPDGDKR | |
| | | | | | DEDGESGGQRSEAEEGEVRTPTKIKELKFLDKPE | |
| | | | | | DVLLKHQASINELKRTLKEPNSKLIHRDRDWERE | |
| | | | | | RRLPSSPASPSPKGTPEKANERAGLREGSEEKV | |
| | | | | | KPPRPRAPESDTGDEDQDQERDTVFLKDNHLAIE | |
| | | | | | RKCSSITVSSTSSLEAEVDFTVIGDYHGSAFEDFS | |
| | | | | | RSLPELDRDKSDSDTEGLLFSRDLNKGAPSQDDE | |
| | | | | | SGGIEDSPDRGACSTPDMPQFEPVKTETMTVSSL | |
| | | | | | AIRKKIEPEAVLQTRVSAMDNTQENSLKSGKGAA | |
| | | | | | AMIPGPQTVATEIRSLSPIIGKDVLTSTYGATAETL | |
| | | | | | STSTTTHVTKTVKGGFSETRIEKRIIITGDEDVDQD | |
| | | | | | QALALAIKEAKLQHPDMLVTKAVVYRETDPSPEE | |
| | | | | | RDKKPQES | |
| 346 | JHUO | NIV | 2.86648284 | EPB41 | MEEKDYSEADGLSERTTPSKAQKSPQKIAKKYKS | Core Human ORF Only |
| | 8576 | O_T | 5 | L1 | AICRVTLLDASEYECEVEKHGRGQVLFDLVCEHL | |
| | | OX | | | NLLEKDYFGLTFCDADSQKNWLDPSKEIKKQIRS | |
| | | | | | SPWNFAFTVKFYPPDPAQLTEDITRYYLCLQLRA | |
| | | | | | DIITGRLPCSFVTHALLGSYAVQAELGDYDAEEHV | |
| | | | | | GNYVSELRFAPNQTRELEERIMELHKTYRGMTPG | |
| | | | | | EAEIHFLENAKKLSMYGVDLHHAKDSEGIDIMLGV | |
| | | | | | CANGLLIYRDRLRINRFAWPKILKISYKRSNFYIKIR | |
| | | | | | PGEYEQFESTIGFKLPNHRSAKRLWKVCIEHHTF | |
| | | | | | FRLVSPEPPPKGFLVMGSKFRYSGRTQAQTRQA | |
| | | | | | SALIDRPAPFFERSSSKRYTMSRSLDGAEFSRPA | |
| | | | | | SVSENHDAGPDGDKRDEDGESGGQRSEAEEGE | |
| | | | | | VRTPTKIKELKFLDKPEDVLLKHQASINELKRTLKE | |
| | | | | | PNSKLIHRDRDWERERRLPSSPASPSPKGTPEKA | |
| | | | | | NERAGLREGSEEKVKPPRPRAPESDTGDEDQDQ | |
| | | | | | ERDTVFLKDNHLAIERKCSSITVSSTSSLEAEVDF | |
| | | | | | TVIGDYHGSAFEDFSRSLPELDRDKSDSDTEGLL | |
| | | | | | FSRDLNKGAPSQDDESGGIEDSPDRGACSTPDM | |
| | | | | | PQFEPVKTETMTVSSLAIRKKIEPEAVLQTRVSAM | |
| | | | | | DNTQENSLKSGKGAAAMIPGPQTVATEIRSLSPII | |
| | | | | | GKDVLTSTYGATAETLSTSTTTHVTKTVKGGFSE | |
| | | | | | TRIEKRIIITGDEDVDQDQALALAIKEAKLQHPDML | |
| | | | | | VTKAVVYRETDPSPEERDKKPQES | |
| 347 | JHUO | NIV | 1.68078213 | KRT33 | MSPILGYWKIKGLVQPTRLLLEYLEEKYEEHLYER | Full Literal Sequence- |
| | 8689 | O_T | 4 | B | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPYNFCLPSLSCRTS | |
| | | | | | CSSRPCVPPSCHGYTLPGACNIPANVSNCNWFC | |
| | | | | | EGSFNGSEKETMQFLNDRLASYLEKVRQLERDN | |
| | | | | | AELENLIRERSQQQEPLLCPSYQSYFKTIEELQQK | |
| | | | | | ILCSKSENARLVVQIDNAKLAADDFRTKYQTEQSL | |
| | | | | | RQLVESDINSLRRILDELTLCRSDLEAQMESLKEE | |
| | | | | | LLSLKQNHEQEVNTLRCQLGDRLNVEVDAAPAVD | |
| | | | | | LNQVLNETRNQYEALVETNRREVEQWFATQTEE | |
| | | | | | LNKQVVSSSEQLQSYQAEIIELRRTVNALEIELQA | |
| | | | | | QHNLRYSLENTLTESEARYSSQLSQVQSLITNVE | |
| | | | | | SQLAEIRSDLERQNQEYQVLLDVRARLECEINTYR | |
| | | | | | SLLESEDCKLPSNPCATTNACEKPIGSCVTNPCG | |
| | | | | | PRSRCGPCNTFGY | |
| 348 | JHUO | NIV | 1.68078213 | KRT33 | MPYNFCLPSLSCRTSCSSRPCVPPSCHGYTLPG | Core Human ORF Only |
| | 8689 | O_T | 4 | B | ACNIPANVSNCNWFCEGSFNGSEKETMQFLNDR | |
| | | OX | | | LASYLEKVRQLERDNAELENLIRERSQQQEPLLC | |
| | | | | | PSYQSYFKTIEELQQKILCSKSENARLVVQIDNAK | |
| | | | | | LAADDFRTKYQTEQSLRQLVESDINSLRRILDELT | |
| | | | | | LCRSDLEAQMESLKEELLSLKQNHEQEVNTLRCQ | |
| | | | | | LGDRLNVEVDAAPAVDLNQVLNETRNQYEALVET | |
| | | | | | NRREVEQWFATQTEELNKQVVSSSEQLQSYQAE | |
| | | | | | IIELRRTVNALEIELQAQHNLRYSLENTLTESEARY | |
| | | | | | SSQLSQVQSLITNVESQLAEIRSDLERQNQEYQV | |
| | | | | | LLDVRARLECEINTYRSLLESEDCKLPSNPCATTN | |
| | | | | | ACEKPIGSCVTNPCGPRSRCGPCNTFGY | |
| 349 | JHUO | NIV | 1.37439307 | NTSR2 | MSPILGYWKIKGLVQPTRLLLEYLEEKYEEHLYER | Full Literal Sequence- |
| | 8892 | O_T | 4 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMETSSPRPPRPSSN | |
| | | | | | PGLSLDARLGVDTRLWAKVLFTALYALIWALGAA | |
| | | | | | GNALSVHVVLKARAGRAGRLRHHVLSLALAGLLL | |
| | | | | | LLVGVPVELYSFVWFHYPVWFGDLGCLAVCQPL | |
| | | | | | CARSLLTPRRTRWLVALSWAASLGLALPMAVIMG | |
| | | | | | QKHELETADGEPEPASRVCTVLVSRTALQVFIQV | |
| | | | | | NVLVSFVLPLALTAFLNGVTVSHLLALCSQVPSTS | |
| | | | | | TPGSSTPSRLELLSEEGLLSFIVWKKTFIQGGQVS | |
| | | | | | LVRHKDVRRIRSLQRSVQVLRAIVVMYVICWLPYH | |
| | | | | | ARRLMYCYVPDDAWTDPLYNFYHYFYMVTNTLF | |
| | | | | | YVSSAVTPLLYNAVSSSFRKLFLEAVSSLCGEHH | |
| | | | | | PMKRLPPKPQSPTLMDTASGFGDPPETRT | |
| 350 | JHUO | NIV | 1.37439307 | NTSR2 | METSSPRPPRPSSNPGLSLDARLGVDTRLWAKV | Core Human ORF Only |
| | 8892 | O_T | 4 | | LFTALYALIWALGAAGNALSVHVVLKARAGRAGR | |
| | | OX | | | LRHHVLSLALAGLLLLLVGVPVELYSFVWFHYPW | |
| | | | | | VFGDLGCLAVCQPLCARSLLTPRRTRWLVALSW | |
| | | | | | AASLGLALPMAVIMGQKHELETADGEPEPASRVC | |
| | | | | | TVLVSRTALQVFIQVNVLVSFVLPLALTAFLNGVTV | |
| | | | | | SHLLALCSQVPSTSTPGSSTPSRLELLSEEGLLSF | |
| | | | | | IVWKKTFIQGGQVSLVRHKDVRRIRSLQRSVQVL | |
| | | | | | RAIVVMYVICWLPYHARRLMYCYVPDDAWTDPLY | |
| | | | | | NFYHYFYMVTNTLFYVSSAVTPLLYNAVSSSFRKL | |
| | | | | | FLEAVSSLCGEHHPMKRLPPKPQSPTLMDTASGF | |
| | | | | | GDPPETRT | |
| 351 | JHUO | NIV | 1.56943934 | SLC22 | MSPILGYWKIKGLVQPTRLLLEYLEEKYEEHLYER | Full Literal Sequence- |
| | 9298 | O_T | 5 | A6 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAFNDLLQQVGGVG | |
| | | | | | RFQQIQVTLVVLPLLLMASHNTLQNFTAAIPTHHC | |
| | | | | | RPPADANLSKNGGLEVWLPRDRQGQPESCLRFT | |
| | | | | | SPQWGLPFLNGTEANGTGATEPCTDGWIYDNST | |
| | | | | | FPSTIVTEWDLVCSHRALRQLAQSLYMVGVLLGA | |
| | | | | | MVFGYLADRLGRRKVLILNYLQTAVSGTCAAFAP | |
| | | | | | NFPIYCAFRLLSGMALAGISLNCMTLNVEWMPIHT | |
| | | | | | RACVGTLIGYVYSLGQFLLAGVAYAVPHWRHLQL | |
| | | | | | LVSAPFFAFFIYSWFFIESARWHSSSGRLDLTLRA | |
| | | | | | LQRVARINGKREEGAKLSMEVLRASLQKELTMGK | |
| | | | | | GQASAMELLRCPTLRHLFLCLSMLWFATSFAYYG | |
| | | | | | LVMDLQGFGVSIYLIQVIFGAVDLPAKLVGFLVINS | |
| | | | | | LGRRPAQMAALLLAGICILLNGVIPQDQSIVRTSLA | |
| | | | | | VLGKGCLAASFNCIFLYTGELYPTMIRQTGMGMG | |
| | | | | | STMARVGSIVSPLVSMTAELYPSMPLFIYGAVPVA | |
| | | | | | ASAVTVLLPETLGQPLPDTVQDLESRKGKQTRQQ | |
| | | | | | QEHQKYMVPLQASAQEKNGL | |
| 352 | JHUO | NIV | 1.56943934 | SLC22 | MAFNDLLQQVGGVGRFQQIQVTLVVLPLLLMASH | Core Human ORF Only |
| | 9298 | O_T | 5 | A6 | NTLQNFTAAIPTHHCRPPADANLSKNGGLEVWLP | |
| | | OX | | | RDRQGQPESCLRFTSPQWGLPFLNGTEANGTGA | |
| | | | | | TEPCTDGWIYDNSTFPSTIVTEWDLVCSHRALRQ | |
| | | | | | LAQSLYMVGVLLGAMVFGYLADRLGRRKVLILNY | |
| | | | | | LQTAVSGTCAAFAPNFPIYCAFRLLSGMALAGISL | |
| | | | | | NCMTLNVEWMPIHTRACVGTLIGYVYSLGQFLLA | |
| | | | | | GVAYAVPHWRHLQLLVSAPFFAFFIYSWFFIESAR | |
| | | | | | WHSSSGRLDLTLRALQRVARINGKREEGAKLSM | |
| | | | | | EVLRASLQKELTMGKGQASAMELLRCPTLRHLFL | |
| | | | | | CLSMLWFATSFAYYGLVMDLQGFGVSIYLIQVIFG | |
| | | | | | AVDLPAKLVGFLVINSLGRRPAQMAALLLAGICILL | |
| | | | | | NGVIPQDQSIVRTSLAVLGKGCLAASFNCIFLYTG | |
| | | | | | ELYPTMIRQTGMGMGSTMARVGSIVSPLVSMTAE | |
| | | | | | LYPSMPLFIYGAVPVAASAVTVLLPETLGQPLPDT | |
| | | | | | VQDLESRKGKQTRQQQEHQKYMVPLQASAQEK | |
| | | | | | NGL | |
| 353 | JHU1 | NIV | 1.99416598 | CSK | MSPILGYWKIKGLVQPTRLLLEYLEEKYEEHLYER | Full Literal Sequence- |
| | 0583 | O_T | 6 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSAIQAAWPSGTECI | |
| | | | | | AKYNFHGTAEQDLPFCKGDVLTIVAVTKDPNWYK | |
| | | | | | AKNKVGREGIIPANYVQKREGVKAGTKLSLMPWF | |
| | | | | | HGKITREQAERLLYPPETGLFLVRESTNYPGDYTL | |
| | | | | | CVSCDGKVEHYRIMYHASKLSIDEEVYFENLMQL | |
| | | | | | VEHYTSDADGLCTRLIKPKVMEGTVAAQDEFYRS | |
| | | | | | GWALNMKELKLLQTIGKGEFGDVMLGDYRGNKV | |
| | | | | | AVKCIKNDATAQAFLAEASVMTQLRHSNLVQLLG | |
| | | | | | VIVEEKGGLYIVTEYMAKGSLVDYLRSRGRSVLG | |
| | | | | | GDCLLKFSLDVCEAMEYLEGNNFVHRDLAARNVL | |
| | | | | | VSEDNVAKVSDFGLTKEASSTQDTGKLPVKWTA | |
| | | | | | PEALREKKFSTKSDVWSFGILLWEIYSFGRVPYP | |
| | | | | | RIPLKDVVPRVEKGYKMDAPDGCPPAVYEVMKN | |
| | | | | | CWHLDAAMRPSFLQLREQLEHIKTHELHL | |
| 354 | JHU1 | NIV | 1.99416598 | CSK | MSAIQAAWPSGTECIAKYNFHGTAEQDLPFCKGD | Core Human ORF Only |
| | 0583 | O_T | 6 | | VLTIVAVTKDPNWYKAKNKVGREGIIPANYVQKRE | |
| | | OX | | | GVKAGTKLSLMPWFHGKITREQAERLLYPPETGL | |
| | | | | | FLVRESTNYPGDYTLCVSCDGKVEHYRIMYHASK | |
| | | | | | LSIDEEVYFENLMQLVEHYTSDADGLCTRLIKPKV | |
| | | | | | MEGTVAAQDEFYRSGWALNMKELKLLQTIGKGE | |
| | | | | | FGDVMLGDYRGNKVAVKCIKNDATAQAFLAEASV | |
| | | | | | MTQLRHSNLVQLLGVIVEEKGGLYIVTEYMAKGSL | |
| | | | | | VDYLRSRGRSVLGGDCLLKFSLDVCEAMEYLEG | |
| | | | | | NNFVHRDLAARNVLVSEDNVAKVSDFGLTKEASS | |
| | | | | | TQDTGKLPVKWTAPEALREKKFSTKSDVWSFGIL | |
| | | | | | LWEIYSFGRVPYPRIPLKDVVPRVEKGYKMDAPD | |
| | | | | | GCPPAVYEVMKNCWHLDAAMRPSFLQLREQLEH | |
| | | | | | IKTHELHL | |
| 355 | JHU1 | NIV | 0.81124279 | SPINK7 | MSPILGYWKIKGLVQPTRLLLEYLEEKYEEHLYER | Full Literal Sequence- |
| | 0919 | O_T | 1 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKITGGLLLLCTVVYF | |
| | | | | | CSSSEAASLSPKKVDCSIYKKYPVVAIPCPITYLPV | |
| | | | | | CGSDYITYGNECHLCTESLKSNGRVQFLHDGSC | |
| 356 | JHU1 | NIV | 0.81124279 | SPINK7 | MKITGGLLLLCTVVYFCSSSEAASLSPKKVDCSIY | Core Human ORF Only |
| | 0919 | O_T | 1 | | KKYPVVAIPCPITYLPVCGSDYITYGNECHLCTESL | |
| | | OX | | | KSNGRVQFLHDGSC | |
| 357 | JHU1 | NIV | 0.52928391 | JADE3 | MSPILGYWKIKGLVQPTRLLLEYLEEKYEEHLYER | Full Literal Sequence- |
| | 1200 | O_T | 4 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKRHRPVSSSDSSD | |
| | | | | | ESPSTSFTSGSMYRIKSKIPNEHKKPAEVFRKDLI | |
| | | | | | SAMKLPDSHHINPDSYYLFADTWKEEWEKGVQV | |
| | | | | | PASPDTVPQPSLRIIAEKVKDVLFIRPRKYIHCSSP | |
| | | | | | DTTEPGYINIMELAASVCRYDLDDMDIFWLQELNE | |
| | | | | | DLAEMGCGPVDENLMEKTVEVLERHCHENMNHA | |
| | | | | | IETEEGLGIEYDEDVICDVCRSPDSEEGNDMVFC | |
| | | | | | DKCNVCVHQACYGILKVPEGSWLCRSCVLGIYPQ | |
| | | | | | CVLCPKKGGALKTTKTGTKWAHVSCALWIPEVSI | |
| | | | | | ACPERMEPITKISHIPPSRWALVCNLCKLKTGACI | |
| | | | | | QCSIKSCITAFHVTCAFEHGLEMKTILDEGDEVKF | |
| | | | | | KSYCLKHSQNRQKLGEAEYPHHRAKEQSQAKSE | |
| | | | | | KTSLRAQKLRELEEEFYSLVRVEDVAAELGMPTL | |
| | | | | | AVDFIYNYWKLKRKSNFNKPLFPPKEDEENGLVQ | |
| | | | | | PKEESIHTRMRMFMHLRQDLERVRNLCYMISRRE | |
| | | | | | KLKLSHNKIQEQIFGLQVQLLNQEIDAGLPLTNALE | |
| | | | | | NSLFYPPPRITLKLKMPKSTPEDHRNSSTETDQQ | |
| | | | | | PHSPDSSSSVHSIRNMQVPQESLEMRTKSYPRY | |
| | | | | | PLESKNNRLLASLSHSRSEAKESSPAWRTPSSEC | |
| | | | | | YHGQSLGKPLVLQAALHGQSSIGNGKSQPNSKF | |
| | | | | | AKSNGLEGSWSGNVTQKDSSSEMFCDQEPVFS | |
| | | | | | PHLVSQGSFRKSTVEHFSRSFKETTNRVWKNTE | |
| | | | | | DLQCYVKPTKNMSPKEQFWGRQVLRRSAGRAP | |
| | | | | | YQENDGYCPDLELSDSEAESDGNKEKVRVRKDS | |
| | | | | | SDRENPPHDSRRDCHGKSKTHPLSHSSMQR | |
| 358 | JHU1 | NIV | 0.52928391 | JADE3 | MKRHRPVSSSDSSDESPSTSFTSGSMYRIKSKIP | Core Human ORF Only |
| | 1200 | O_T | 4 | | NEHKKPAEVFRKDLISAMKLPDSHHINPDSYYLFA | |
| | | OX | | | DTWKEEWEKGVQVPASPDTVPQPSLRIIAEKVKD | |
| | | | | | VLFIRPRKYIHCSSPDTTEPGYINIMELAASVCRYD | |
| | | | | | LDDMDIFWLQELNEDLAEMGCGPVDENLMEKTV | |
| | | | | | EVLERHCHENMNHAIETEEGLGIEYDEDVICDVC | |
| | | | | | RSPDSEEGNDMVFCDKCNVCVHQACYGILKVPE | |
| | | | | | GSWLCRSCVLGIYPQCVLCPKKGGALKTTKTGTK | |
| | | | | | WAHVSCALWIPEVSIACPERMEPITKISHIPPSRW | |
| | | | | | ALVCNLCKLKTGACIQCSIKSCITAFHVTCAFEHGL | |
| | | | | | EMKTILDEGDEVKFKSYCLKHSQNRQKLGEAEYP | |
| | | | | | HHRAKEQSQAKSEKTSLRAQKLRELEEEFYSLVR | |
| | | | | | VEDVAAELGMPTLAVDFIYNYWKLKRKSNFNKPL | |
| | | | | | FPPKEDEENGLVQPKEESIHTRMRMFMHLRQDL | |
| | | | | | ERVRNLCYMISRREKLKLSHNKIQEQIFGLQVQLL | |
| | | | | | NQEIDAGLPLTNALENSLFYPPPRITLKLKMPKST | |
| | | | | | PEDHRNSSTETDQQPHSPDSSSSVHSIRNMQVP | |
| | | | | | QESLEMRTKSYPRYPLESKNNRLLASLSHSRSEA | |
| | | | | | KESSPAWRTPSSECYHGQSLGKPLVLQAALHGQ | |
| | | | | | SSIGNGKSQPNSKFAKSNGLEGSWSGNVTQKDS | |
| | | | | | SSEMFCDQEPVFSPHLVSQGSFRKSTVEHFSRS | |
| | | | | | FKETTNRWVKNTEDLQCYVKPTKNMSPKEQFWG | |
| | | | | | RQVLRRSAGRAPYQENDGYCPDLELSDSEAESD | |
| | | | | | GNKEKVRVRKDSSDRENPPHDSRRDCHGKSKT | |
| | | | | | HPLSHSSMQR | |
| 359 | JHU1 | NIV | 2.46365554 | HYAL1 | MSPILGYWKIKGLVQPTRLLLEYLEEKYEEHLYER | Full Literal Sequence- |
| | 1837 | O_T | 6 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAHLLPICALFLTLL | |
| | | | | | DMAQGFRGPLLPNRPFTTVWNANTQWCLERHG | |
| | | | | | VDVDVSVFDVVANPGQTFRGPDMTIFYSSQLGTY | |
| | | | | | PYYTPTGEPVFGGLPQNASLIAHLARTFQDILAAIP | |
| | | | | | APDFSGLAVIDWEAWRPRWAFNWDTKDIYRQRS | |
| | | | | | RALVQAQHPDWPAPQVEAVAQDQFQGAARAWM | |
| | | | | | AGTLQLGRALRPRGLWGFYGFPDCSG | |
| 360 | JHU1 | NIV | 2.46365554 | HYAL1 | MAAHLLPICALFLTLLDMAQGFRGPLLPNRPFTTV | Core Human ORF Only |
| | 1837 | O_T | 6 | | WNANTQWCLERHGVDVDVSVFDVVANPGQTFR | |
| | | OX | | | GPDMTIFYSSQLGTYPYYTPTGEPVFGGLPQNAS | |
| | | | | | LIAHLARTFQDILAAIPAPDFSGLAVIDWEAWRPR | |
| | | | | | WAFNWDTKDIYRQRSRALVQAQHPDWPAPQVE | |
| | | | | | AVAQDQFQGAARAWMAGTLQLGRALRPRGLWG | |
| | | | | | FYGFPDCSG | |
| 361 | JHU1 | NIV | 0.44067707 | MRAS | MSPILGYWKIKGLVQPTRLLLEYLEEKYEEHLYER | Full Literal Sequence- |
| | 3076 | O_T | 1 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMATSAVPSDNLPTYK | |
| | | | | | LVVVGDGGVGKSALTIQFFQKIFVPDYDPTIEDSY | |
| | | | | | LKHTEIDNQWAILDVLDTAGQEEFSAMREQYMRT | |
| | | | | | GDGFLIVYSVTDKASFEHVDRFHQLILRVKDRESF | |
| | | | | | PMILVANKVDLMHLRKITREQGKEMATKHNIPYIE | |
| | | | | | TSAKDPPLNVDKAFHDLVRVIRQQIPEKSQKKKKK | |
| | | | | | TKWRGDRATGTHKLQCVIL | |
| 362 | JHU1 | NIV | 0.44067707 | MRAS | MATSAVPSDNLPTYKLVVVGDGGVGKSALTIQFF | Core Human ORF Only |
| | 3076 | O_T | 1 | | QKIFVPDYDPTIEDSYLKHTEIDNQWAILDVLDTAG | |
| | | OX | | | QEEFSAMREQYMRTGDGFLIVYSVTDKASFEHV | |
| | | | | | DRFHQLILRVKDRESFPMILVANKVDLMHLRKITR | |
| | | | | | EQGKEMATKHNIPYIETSAKDPPLNVDKAFHDLV | |
| | | | | | RVIRQQIPEKSQKKKKKTKWRGDRATGTHKLQCV | |
| | | | | | IL | |
| 363 | JHU1 | NIV | 0.86465563 | TOP3B | MSPILGYWKIKGLVQPTRLLLEYLEEKYEEHLYER | Full Literal Sequence- |
| | 3972 | O_T | 3 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMKTVLMVAEKPSLA | |
| | | | | | QSIAKILSRGSLSSHKGLNGACSVHEYTGTFAGQ | |
| | | | | | PVRFKMTSVCGHVMTLDFLGKYNKWDKVDPAEL | |
| | | | | | FSQAPTEKKEANPKLNMVKFLQVEGRGCDYIVLW | |
| | | | | | LDCDKEGENICFEVLDAVLPVMNKAHGGEKTVFR | |
| | | | | | ARFSSITDTDICNAMACLGEPDHNEALSVDARQE | |
| | | | | | LDLRIGCAFTRFQTKYFQGKYGDLDSSLISFGPCQ | |
| | | | | | TPTLGFCVERHDKIQSFKPETYWVLQAKVNTDKD | |
| | | | | | RSLLLDWDRVRVFDREIAQMFLNMTKLEKEAQVE | |
| | | | | | ATSRKEKAKQRPLALNTVEMLRVASSSLGMGPQ | |
| | | | | | HAMQTAERLYTQGYISYPRTETTHYPENFDLKGS | |
| | | | | | LRQQANHPYWADTVKRLLAEGINRPRKGHDAGD | |
| | | | | | HPPITPMKSATEAELGGDAWRLYEYITRHFIATVS | |
| | | | | | HDCKYLQSTISFRIGPELFTCSGKTVLSPGFTEVM | |
| | | | | | PWQSVPLEESLPTCQRGDAFPVGEVKMLEKQTN | |
| | | | | | PPDYLTEAELITLMEKHGIGTDASIPVHINNICQRN | |
| | | | | | YVTVESGRRLKPTNLGIVLVHGYYKIDAELVLPTIR | |
| | | | | | SAVEKQLNLIAQGKADYRQVLGHTLDVFKRKFHY | |
| | | | | | FVDSIAGMDELMEVSFSPLAATGKPLSRCGKCHR | |
| | | | | | FMKYIQAKPSRLHCSHCDETYTLPQNGTIKLYKEL | |
| | | | | | RCPLDDFELVLWSSGSRGKSYPLCPYCYNHPPF | |
| | | | | | RDMKKGMGCNECTHPSCQHSLSMLGIGQCVEC | |
| | | | | | ESGVLVLDPTSGPKWKVACNKCNVVAHCFENAH | |
| | | | | | RVRVSADTCSVCEAALLDVDFNKAKSPLPGDETQ | |
| | | | | | HMGCVFCDPVFQELVELKHAASCHPMHRGGPG | |
| | | | | | RRQGRGRGRARRPPGKPNPRRPKDKMSALAAY | |
| | | | | | FV | |
| 364 | JHU1 | NIV | 0.86465563 | TOP3B | MKTVLMVAEKPSLAQSIAKILSRGSLSSHKGLNGA | Core Human ORF Only |
| | 3972 | O_T | 3 | | CSVHEYTGTFAGQPVRFKMTSVCGHVMTLDFLG | |
| | | OX | | | KYNKWDKVDPAELFSQAPTEKKEANPKLNMVKF | |
| | | | | | LQVEGRGCDYIVLWLDCDKEGENICFEVLDAVLP | |
| | | | | | VMNKAHGGEKTVFRARFSSITDTDICNAMACLGE | |
| | | | | | PDHNEALSVDARQELDLRIGCAFTRFQTKYFQGK | |
| | | | | | YGDLDSSLISFGPCQTPTLGFCVERHDKIQSFKPE | |
| | | | | | TYVWLQAKVNTDKDRSLLLDWDRVRVFDREIAQ | |
| | | | | | MFLNMTKLEKEAQVEATSRKEKAKQRPLALNTVE | |
| | | | | | MLRVASSSLGMGPQHAMQTAERLYTQGYISYPR | |
| | | | | | TETTHYPENFDLKGSLRQQANHPYWADTVKRLL | |
| | | | | | AEGINRPRKGHDAGDHPPITPMKSATEAELGGDA | |
| | | | | | WRLYEYITRHFIATVSHDCKYLQSTISFRIGPELFT | |
| | | | | | CSGKTVLSPGFTEVMPWQSVPLEESLPTCQRGD | |
| | | | | | AFPVGEVKMLEKQTNPPDYLTEAELITLMEKHGIG | |
| | | | | | TDASIPVHINNICQRNYVTVESGRRLKPTNLGIVLV | |
| | | | | | HGYYKIDAELVLPTIRSAVEKQLNLIAQGKADYRQ | |
| | | | | | VLGHTLDVFKRKFHYFVDSIAGMDELMEVSFSPL | |
| | | | | | AATGKPLSRCGKCHRFMKYIQAKPSRLHCSHCD | |
| | | | | | ETYTLPQNGTIKLYKELRCPLDDFELVLWSSGSR | |
| | | | | | GKSYPLCPYCYNHPPFRDMKKGMGCNECTHPS | |
| | | | | | CQHSLSMLGIGQCVECESGVLVLDPTSGPKWKV | |
| | | | | | ACNKCNVVAHCFENAHRVRVSADTCSVCEAALL | |
| | | | | | DVDFNKAKSPLPGDETQHMGCVFCDPVFQELVE | |
| | | | | | LKHAASCHPMHRGGPGRRQGRGRGRARRPPGK | |
| | | | | | PNPRRPKDKMSALAAYFV | |
| 365 | JHU1 | NIV | 1.42707161 | GTPBP | MSPILGYWKIKGLVQPTRLLLEYLEEKYEEHLYER | Full Literal Sequence- |
| | 4010 | O_T | 5 | 6 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTRAEWQVAEATAL | |
| | | | | | VHTLDGWSVVQTMVVSTKTPDRKLIFGKGNFEHL | |
| | | | | | TEKIRGSPDVTCVFLNVERMAAPTKKELEAAWGV | |
| | | | | | EVFDRFTVVLHIFRCNARTKEARLQVALAEMPLH | |
| | | | | | RSNLKRDVAHLYRGVGSRYIMGSGESFMQLQQR | |
| | | | | | LLREKEAKIRKALDRLRKKRHLLRRQRTRREFPVI | |
| | | | | | SVVGYTNCGKTTLIKALTGDAAIQPRDQLFATLDV | |
| | | | | | TAHAGTLPSRMTVLYVDTIGFLSQLPHGLIESFSA | |
| | | | | | TLEDVAHSDLILHVRDVSHPEAELQKCSVLSTLRG | |
| | | | | | LQLPAPLLDSMVEVHNKVDLVPGYSPTEPNVVPV | |
| | | | | | SALRGHGLQELKAELDAAVLKATGRQILTLRVRLA | |
| | | | | | GAQLSWLYKEATVQEVDVIPEDGAADVRVIISNSA | |
| | | | | | YGKFRKLFPG | |
| 366 | JHU1 | NIV | 1.42707161 | GTPBP | MTRAEWQVAEATALVHTLDGWSVVQTMVVSTKT | Core Human ORF Only |
| | 4010 | O_T | 5 | 6 | PDRKLIFGKGNFEHLTEKIRGSPDVTCVFLNVERM | |
| | | OX | | | AAPTKKELEAAWGVEVFDRFTVVLHIFRCNARTK | |
| | | | | | EARLQVALAEMPLHRSNLKRDVAHLYRGVGSRYI | |
| | | | | | MGSGESFMQLQQRLLREKEAKIRKALDRLRKKR | |
| | | | | | HLLRRQRTRREFPVISVVGYTNCGKTTLIKALTGD | |
| | | | | | AAIQPRDQLFATLDVTAHAGTLPSRMTVLYVDTIG | |
| | | | | | FLSQLPHGLIESFSATLEDVAHSDLILHVRDVSHP | |
| | | | | | EAELQKCSVLSTLRGLQLPAPLLDSMVEVHNKVD | |
| | | | | | LVPGYSPTEPNVVPVSALRGHGLQELKAELDAAV | |
| | | | | | LKATGRQILTLRVRLAGAQLSWLYKEATVQEVDVI | |
| | | | | | PEDGAADVRVIISNSAYGKFRKLFPG | |
| 367 | JHU1 | NIV | 1.91314876 | ZNF25 | MSPILGYWKIKGLVQPTRLLLEYLEEKYEEHLYER | Full Literal Sequence- |
| | 5476 | O_T | 3 | 4 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPGPPRSLEMGLLT | |
| | | | | | FRDVAIEFSLEEWQHLDIAQQNLYRNVMLENYRN | |
| | | | | | LAFLGIAVSKPDLITCLEQGKEPWNMKRHEMVDE | |
| | | | | | PPGMCPHFAQGLWPEQGMEDSFQKAILRRYGKY | |
| | | | | | GHENLQLRKGCKSVDEYKVNKEGYNGLNQCFTT | |
| | | | | | AQSKVFQCDKYLKVFYKFLNSNRPKIRHTEKKSF | |
| | | | | | KCKKRVKLFCMLSHKTQHKSIYHREKSYKCKECG | |
| | | | | | KTFNWSSTLTNHRKIYTEEKPYKCEEYNKSPKQL | |
| | | | | | STLTTHEIIHAGEKLYKCEECGEAFNRSSNLTTHKI | |
| | | | | | IHTGEKPYKCEECGKAFIWSSTLTEHKKIHTRKKP | |
| | | | | | YKCEECGKAFIWSSTLTRHKRMHTGEKPYKCEE | |
| | | | | | CGKAFSQSSTLTTHKIIHTGEKRYKCLECGKAFKQ | |
| | | | | | LSTLTTHKIIHVGEKLYKCEECGKGFNRSSNLTTH | |
| | | | | | KIIHTGEKPYKCEECGKAFIWSSTLTKHKRIHTREK | |
| | | | | | PYKCEECGKAFIWSSTLTRHKRMHTGEKPYKCE | |
| | | | | | ECGKSFSQSSTLTTHKIIHTGEKPYKCEECGKAFN | |
| | | | | | WSSTLTKHNIIHTEEKPYKCEKCGKAFKQSSILTN | |
| | | | | | HKRIHTGEKPYKCEECGKSFNRSSTFTKHKVIHT | |
| | | | | | GVKPYKCEECGKAFFWSSTLTKHKRIHTGEQPYK | |
| | | | | | WEKFGKAFNRSSHLTTDKITHWREILQV | |
| 368 | JHU1 | NIV | 1.91314876 | ZNF25 | MPGPPRSLEMGLLTFRDVAIEFSLEEWQHLDIAQ | Core Human ORF Only |
| | 5476 | O_T | 3 | 4 | QNLYRNVMLENYRNLAFLGIAVSKPDLITCLEQGK | |
| | | OX | | | EPWNMKRHEMVDEPPGMCPHFAQGLWPEQGM | |
| | | | | | EDSFQKAILRRYGKYGHENLQLRKGCKSVDEYKV | |
| | | | | | NKEGYNGLNQCFTTAQSKVFQCDKYLKVFYKFLN | |
| | | | | | SNRPKIRHTEKKSFKCKKRVKLFCMLSHKTQHKSI | |
| | | | | | YHREKSYKCKECGKTFNWSSTLTNHRKIYTEEKP | |
| | | | | | YKCEEYNKSPKQLSTLTTHEIIHAGEKLYKCEECG | |
| | | | | | EAFNRSSNLTTHKIIHTGEKPYKCEECGKAFIWSS | |
| | | | | | TLTEHKKIHTRKKPYKCEECGKAFIWSSTLTRHKR | |
| | | | | | MHTGEKPYKCEECGKAFSQSSTLTTHKIIHTGEK | |
| | | | | | RYKCLECGKAFKQLSTLTTHKIIHVGEKLYKCEEC | |
| | | | | | GKGFNRSSNLTTHKIIHTGEKPYKCEECGKAFIWS | |
| | | | | | STLTKHKRIHTREKPYKCEECGKAFIWSSTLTRHK | |
| | | | | | RMHTGEKPYKCEECGKSFSQSSTLTTHKIIHTGE | |
| | | | | | KPYKCEECGKAFNWSSTLTKHNIIHTEEKPYKCE | |
| | | | | | KCGKAFKQSSILTNHKRIHTGEKPYKCEECGKSF | |
| | | | | | NRSSTFTKHKVIHTGVKPYKCEECGKAFFWSSTL | |
| | | | | | TKHKRIHTGEQPYKWEKFGKAFNRSSHLTTDKIT | |
| | | | | | HWREILQV | |
| 369 | JHU1 | NIV | 0.7460258 | ZNF62 | MSPILGYWKIKGLVQPTRLLLEYLEEKYEEHLYER | Full Literal Sequence- |
| | 5859 | O_T | 8 | 4 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEPKPATKKATRTKA | |
| | | | | | ISEDLSQEAILEKLTENGLWDSRMEGLWKWNDRI | |
| | | | | | LRLQNNQENHLSQRIIPLKKTPTSQRGFRFESILIP | |
| | | | | | EPGIATEELHSRCQTQEENFTENLNLITDTHLGKII | |
| | | | | | CKEMKGSKAIRQTSELTLGKKSNNKEKPYKCSTC | |
| | | | | | EKAFHYRSLLIQHQRTHTKEKPYECNECGKTFSQ | |
| | | | | | PSYLSQHKKIHTGEKPYKCNECGKAFIASSSLMV | |
| | | | | | HQRIHTKEKPYQCNVCGKSFSQCARLNQHQRIQ | |
| | | | | | TGEKPYKCSECGKAFSDKSKLARHQETHNGEKP | |
| | | | | | YKCDDCGKAFRNKSYLSVHQKTHTEEKPYQCNE | |
| | | | | | CGKSFKNTTIFNVHQRIHTGEKPFRCNECGKAYR | |
| | | | | | SNSSLIVHIRTHTGEKPYECNECGKAFNRIANFTE | |
| | | | | | HQRIHTGEKPYKCNECGKAFINYSCLTVHHRMHT | |
| | | | | | GEKPYKCTECGKAFMRSSSLIIHQRIHTEEKPYLC | |
| | | | | | NECGESFRIKSHLTVHQRIHTGEKPYKCTDCERA | |
| | | | | | FTKMVNLKEHQKIHTGVKPYKCYDCGKSFRTKSY | |
| | | | | | LIVHQRTHTGEKPYKCNECEKAFTNTSQLTVHQR | |
| | | | | | RHTGEKPYKCNECGKVFTSNSGFNTHQRTHTGE | |
| | | | | | KPFKCNDCGKAFSQMVHVTEHQKIHTGEKPYKC | |
| | | | | | DVCGKAFRRGSYLTVHWRTHTGEKPYTCKECGK | |
| | | | | | GCITLSQLTLHQRIHTGERPYKCEECGKAFRTNS | |
| | | | | | DFTVHLRMHTGEKPYKCNECGKAFRSSSSLTVH | |
| | | | | | QRIHQRETQLI | |
| 370 | JHU1 | NIV | 0.7460258 | ZNF62 | MEPKPATKKATRTKAISEDLSQEAILEKLTENGLW | Core Human ORF Only |
| | 5859 | O_T | 8 | 4 | DSRMEGLWKWNDRILRLQNNQENHLSQRIIPLKK | |
| | | OX | | | TPTSQRGFRFESILIPEPGIATEELHSRCQTQEEN | |
| | | | | | FTENLNLITDTHLGKIICKEMKGSKAIRQTSELTLG | |
| | | | | | KKSNNKEKPYKCSTCEKAFHYRSLLIQHQRTHTK | |
| | | | | | EKPYECNECGKTFSQPSYLSQHKKIHTGEKPYKC | |
| | | | | | NECGKAFIASSSLMVHQRIHTKEKPYQCNVCGKS | |
| | | | | | FSQCARLNQHQRIQTGEKPYKCSECGKAFSDKS | |
| | | | | | KLARHQETHNGEKPYKCDDCGKAFRNKSYLSVH | |
| | | | | | QKTHTEEKPYQCNECGKSFKNTTIFNVHQRIHTG | |
| | | | | | EKPFRCNECGKAYRSNSSLIVHIRTHTGEKPYEC | |
| | | | | | NECGKAFNRIANFTEHQRIHTGEKPYKCNECGKA | |
| | | | | | FINYSCLTVHHRMHTGEKPYKCTECGKAFMRSSS | |
| | | | | | LIIHQRIHTEEKPYLCNECGESFRIKSHLTVHQRIH | |
| | | | | | TGEKPYKCTDCERAFTKMVNLKEHQKIHTGVKPY | |
| | | | | | KCYDCGKSFRTKSYLIVHQRTHTGEKPYKCNECE | |
| | | | | | KAFTNTSQLTVHQRRHTGEKPYKCNECGKVFTS | |
| | | | | | NSGFNTHQRTHTGEKPFKCNDCGKAFSQMVHVT | |
| | | | | | EHQKIHTGEKPYKCDVCGKAFRRGSYLTVHWRT | |
| | | | | | HTGEKPYTCKECGKGCITLSQLTLHQRIHTGERP | |
| | | | | | YKCEECGKAFRTNSDFTVHLRMHTGEKPYKCNE | |
| | | | | | CGKAFRSSSSLTVHQRIHQRETQLI | |
| 371 | JHU1 | NIV | 0.23594282 | TTC31 | MSPILGYWKIKGLVQPTRLLLEYLEEKYEEHLYER | Full Literal Sequence- |
| | 5952 | O_T | 1 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAPIPKTVGRIKLDC | |
| | | | | | SLRPSCPLEVAAAPKLCKEFGPEDYGEEDIVDFL | |
| | | | | | RRLVESDPQGLHRIHVDGSSGRLQLWHHDYLLG | |
| | | | | | HLDDEGKSTGQSDRGKGAEGLGTYCGLRKSFLY | |
| | | | | | PPQESEPCPQSPSASATFPSVSDSLLQVAMPQKL | |
| | | | | | LVTEEEANRLAEELVAEEERMKQKAEKKRLKKKR | |
| | | | | | QKERKRQERLEQYCGEPKASTTSDGDESPPSSP | |
| | | | | | GNPVQGQCGEEEDSLDLSSTFVSLALRKVGDWP | |
| | | | | | LSARREKGLNQEPQGRGLALQKMGQEEESPPRE | |
| | | | | | ERPQQSPKEKDLGRLRPQDLLDFAPYPQASPGLL | |
| | | | | | AAALQQSQELAKLGTSFAQNGFYHEAVVLFTQAL | |
| | | | | | KLNPQDHR | |
| 372 | JHU1 | NIV | 0.23594282 | TTC31 | MAPIPKTVGRIKLDCSLRPSCPLEVAAAPKLCKEF | Core Human ORF Only |
| | 5952 | O_T | 1 | | GPEDYGEEDIVDFLRRLVESDPQGLHRIHVDGSS | |
| | | OX | | | GRLQLWHHDYLLGHLDDEGKSTGQSDRGKGAE | |
| | | | | | GLGTYCGLRKSFLYPPQESEPCPQSPSASATFPS | |
| | | | | | VSDSLLQVAMPQKLLVTEEEANRLAEELVAEEER | |
| | | | | | MKQKAEKKRLKKKRQKERKRQERLEQYCGEPKA | |
| | | | | | STTSDGDESPPSSPGNPVQGQCGEEEDSLDLSS | |
| | | | | | TFVSLALRKVGDWPLSARREKGLNQEPQGRGLA | |
| | | | | | LQKMGQEEESPPREERPQQSPKEKDLGRLRPQD | |
| | | | | | LLDFAPYPQASPGLLAAALQQSQELAKLGTSFAQ | |
| | | | | | NGFYHEAVVLFTQALKLNPQDHR | |
| 373 | JHU1 | NIV | 0.79596095 | TSFM | MSPILGYWKIKGLVQPTRLLLEYLEEKYEEHLYER | Full Literal Sequence- |
| | 6337 | O_T | 2 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSLLRSLRVFLVART | |
| | | | | | GSYPAGSLLRQSPQPRHTFYAGPRLSASASSKEL | |
| | | | | | LMKLRRKTGYSFVNCKKALETCGGDLKQAEIWLH | |
| | | | | | KEAQKEGWSKAAKLQGRKTKEGLIGLLQEGNTTV | |
| | | | | | LVEVNCETDFVSRNLKFQLLVQQVALGTMMHCQ | |
| | | | | | TLKDQPSAYSKGFLNSSELSGLPAGPDREGSLKD | |
| | | | | | QLALAIGKLGETMILKRAAVWKVPSGFYVGSYVH | |
| | | | | | GAMQSPSLHKLVLGKYGALVICETSEQKTNLEDV | |
| | | | | | GRRLGQHVVGMAPLSVGSLDDEPGGEAETKMLS | |
| | | | | | QPYLLDPSITLGQYVQPQGVSVVDFVRFECGEGE | |
| | | | | | EAAETE | |
| 374 | JHU1 | NIV | 0.79596095 | TSFM | MSLLRSLRVFLVARTGSYPAGSLLRQSPQPRHTF | Core Human ORF Only |
| | 6337 | O_T | 2 | | YAGPRLSASASSKELLMKLRRKTGYSFVNCKKAL | |
| | | OX | | | ETCGGDLKQAEIWLHKEAQKEGWSKAAKLQGRK | |
| | | | | | TKEGLIGLLQEGNTTVLVEVNCETDFVSRNLKFQL | |
| | | | | | LVQQVALGTMMHCQTLKDQPSAYSKGFLNSSEL | |
| | | | | | SGLPAGPDREGSLKDQLALAIGKLGETMILKRAA | |
| | | | | | WVKVPSGFYVGSYVHGAMQSPSLHKLVLGKYGA | |
| | | | | | LVICETSEQKTNLEDVGRRLGQHVVGMAPLSVGS | |
| | | | | | LDDEPGGEAETKMLSQPYLLDPSITLGQYVQPQG | |
| | | | | | VSVVDFVRFECGEGEEAAETE | |
| 375 | JHU1 | NIV | 0.76468070 | MSGN1 | MSPILGYWKIKGLVQPTRLLLEYLEEKYEEHLYER | Full Literal Sequence- |
| | 6662 | O_T | 8 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDNLRETFLSLEDGL | |
| | | | | | GSSDSPGLLSSWDWKDRAGPFELNQASPSQSLS | |
| | | | | | PAPSLESYSSSPCPAVAGLPCEHGGASSGGSEG | |
| | | | | | CSVGGASGLVEVDYNMLAFQPTHLQGGGGPKA | |
| | | | | | QKGTKVRMSVQRRRKASEREKLRMRTLADALHT | |
| | | | | | LRNYLPPVYSQRGQPLTKIQTLKYTIKYIGELTDLL | |
| | | | | | NRGREPRAQSA | |
| 376 | JHU1 | NIV | 0.76468070 | MSGN1 | MDNLRETFLSLEDGLGSSDSPGLLSSWDWKDRA | Core Human ORF Only |
| | 6662 | O_T | 8 | | GPFELNQASPSQSLSPAPSLESYSSSPCPAVAGL | |
| | | OX | | | PCEHGGASSGGSEGCSVGGASGLVEVDYNMLA | |
| | | | | | FQPTHLQGGGGPKAQKGTKVRMSVQRRRKASE | |
| | | | | | REKLRMRTLADALHTLRNYLPPVYSQRGQPLTKI | |
| | | | | | QTLKYTIKYIGELTDLLNRGREPRAQSA | |
| 377 | JHU1 | NIV | 1.0196905 | ARHGA | MSPILGYWKIKGLVQPTRLLLEYLEEKYEEHLYER | Full Literal Sequence- |
| | 7082 | O_T | 8 | P8 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVILRDKIRFYEELQR | |
| | | | | | DKAAAAAVLGAVRKRPSVVPMAGQDPALSTSHP | |
| | | | | | FYDVARHGILQVAGDDRFGRRVVTFSCCRMPPS | |
| | | | | | HELDHQRLLEYLKYTLDQYVENDYTIVYFHYGLNS | |
| | | | | | RNKPSLGWLQSAYKEFDRKYKKNLKALYVVHPTS | |
| | | | | | FIKVLWNILKPLISHKFGKKVIYFNYLSELHEHLKY | |
| | | | | | DQLVIPPEVLRYDEKLQSLHEGRTPPPTKTPPPR | |
| | | | | | PPLPTQQFGVSLQYLKDKNQGELIPPVLRFTVTYL | |
| | | | | | REKGLRTEGLFRRSASVQTVREIQRLYNQGKPVN | |
| | | | | | FDDYGDIHIPAVILKTFLRELPQPLLTFQAYEQILGI | |
| | | | | | TCVESSLRVTGCRQILRSLPEHNYVVLRYLMGFL | |
| | | | | | HAVSRESIFNKMNSSNLACVFGLNLIWPSQGVSS | |
| | | | | | LSALVPLNMFTELLIEYYEKIFSTPEAPGEHGLAP | |
| | | | | | WEQGSRAAPLQEAVPRTQATGLTKPTLPPSPLM | |
| | | | | | AARRRLNPAFLYKVVKASS | |
| 378 | JHU1 | NIV | 1.0196905 | ARHGA | MVILRDKIRFYEELQRDKAAAAAVLGAVRKRPSVV | Core Human ORF Only |
| | 7082 | O_T | 8 | P8 | PMAGQDPALSTSHPFYDVARHGILQVAGDDRFG | |
| | | OX | | | RRVVTFSCCRMPPSHELDHQRLLEYLKYTLDQYV | |
| | | | | | ENDYTIVYFHYGLNSRNKPSLGWLQSAYKEFDRK | |
| | | | | | YKKNLKALYVVHPTSFIKVLWNILKPLISHKFGKKV | |
| | | | | | IYFNYLSELHEHLKYDQLVIPPEVLRYDEKLQSLH | |
| | | | | | EGRTPPPTKTPPPRPPLPTQQFGVSLQYLKDKNQ | |
| | | | | | GELIPPVLRFTVTYLREKGLRTEGLFRRSASVQTV | |
| | | | | | REIQRLYNQGKPVNFDDYGDIHIPAVILKTFLRELP | |
| | | | | | QPLLTFQAYEQILGITCVESSLRVTGCRQILRSLPE | |
| | | | | | HNYVVLRYLMGFLHAVSRESIFNKMNSSNLACVF | |
| | | | | | GLNLIWPSQGVSSLSALVPLNMFTELLIEYYEKIFS | |
| | | | | | TPEAPGEHGLAPWEQGSRAAPLQEAVPRTQATG | |
| | | | | | LTKPTLPPSPLMAARRRL | |
| 379 | JHU1 | NIV | 1.41421139 | TNFRS | MSPILGYWKIKGLVQPTRLLLEYLEEKYEEHLYER | Full Literal Sequence- |
| | 7198 | O_T | 6 | F25 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEQRPRGCAAVAAA | |
| | | | | | LLLVLLGARAQGGTRSPRCDCAGDFHKKIGLFCC | |
| | | | | | RGCPAGHYLKAPCTEPCGNSTCLVCPQDTFLAW | |
| | | | | | ENHHNSECARCQACDEQASQVALENCSAVADTR | |
| | | | | | CGCKPGWFVECQVSQCVSSSPFYCQPCLDCGA | |
| | | | | | LHRHTRLLCSRRDTDCGTCLPGFYEHGDGCVSC | |
| | | | | | PTSTLGSCPERCAAVCGWRQMFWVQVLLAGLVV | |
| | | | | | PLLLGATLTYTYRHCWPHKPLVTADEAGMEALTP | |
| | | | | | PPATHLSPLDSAHTLLAPPDSSEKICTVQLVGNS | |
| | | | | | WTPGYPETQEALCPQVTWSWDQLPSRALGPAA | |
| | | | | | APTLSPESPAGSPAMMLQPGPQLYDVMDAVPAR | |
| | | | | | RWKEFVRTLGLREAEIEAVEVEIGRFRDQQYEML | |
| | | | | | KRWRQQQPAGLGAVYAALERMGLDGCVEDLRS | |
| | | | | | RLQRGPNPAFLYKVVKASS | |
| 380 | JHU1 | NIV | 1.41421139 | TNFRS | MEQRPRGCAAVAAALLLVLLGARAQGGTRSPRC | Core Human ORF Only |
| | 7198 | O_T | 6 | F25 | DCAGDFHKKIGLFCCRGCPAGHYLKAPCTEPCG | |
| | | OX | | | NSTCLVCPQDTFLAWENHHNSECARCQACDEQA | |
| | | | | | SQVALENCSAVADTRCGCKPGWFVECQVSQCV | |
| | | | | | SSSPFYCQPCLDCGALHRHTRLLCSRRDTDCGT | |
| | | | | | CLPGFYEHGDGCVSCPTSTLGSCPERCAAVCGW | |
| | | | | | RQMFWVQVLLAGLVVPLLLGATLTYTYRHCWPH | |
| | | | | | KPLVTADEAGMEALTPPPATHLSPLDSAHTLLAPP | |
| | | | | | DSSEKICTVQLVGNSWTPGYPETQEALCPQVTW | |
| | | | | | SWDQLPSRALGPAAAPTLSPESPAGSPAMMLQP | |
| | | | | | GPQLYDVMDAVPARRWKEFVRTLGLREAEIEAVE | |
| | | | | | VEIGRFRDQQYEMLKRWRQQQPAGLGAVYAALE | |
| | | | | | RMGLDGCVEDLRSRLQRGP | |
| 381 | JHU1 | NIV | 1.10370141 | MARK4 | MSPILGYWKIKGLVQPTRLLLEYLEEKYEEHLYER | Full Literal Sequence- |
| | 7311 | O_T | 9 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSSRTVLAPGNDRN | |
| | | | | | SDTHGTLGSGRSSDKGPSWSSRSLGARCRNSIA | |
| | | | | | SCPEEQPHVGNYRLLRTIGKGNFAKVKLARHILTG | |
| | | | | | REVAIKIIDKTQLNPSSLQKLFREVRIMKGLNHPNI | |
| | | | | | VKLFEVIETEKTLYLVMEYASAGEVFDYLVSHGR | |
| | | | | | MKEKEARAKFRQIVSAVHYCHQKNIVHRDLKAEN | |
| | | | | | LLLDAEANIKIADFGFSNEFTLGSKLDTFCGSPPY | |
| | | | | | AAPELFQGKKYDGPEVDIWSLGVILYTLVSGSLPF | |
| | | | | | DGHNLKELRERVLRGKYRVPFYMSTDCESILRRF | |
| | | | | | LVLNPAKRCTLEQIMKDKWINIGYEGEELKPYTEP | |
| | | | | | EEDFGDTKRIEVMVGMGYTREEIKESLTSQKYNE | |
| | | | | | VTATYLLLGRKTEEGGDRGAPGLALARVRAPSDT | |
| | | | | | TNGTSSSKGTSHSKGQRSSSSTYHRQRRHSDFC | |
| | | | | | GPSPAPLHPKRSPTSTGEAELKEERLPGRKASCS | |
| | | | | | TAGSGSRGLPPSSPMVSSAHNPNKAEIPERRKD | |
| | | | | | STSTPNNLPPSMMTRRNTYVCTERPGAERPSLLP | |
| | | | | | NGKENSSGTPRVPPASPSSHSLAPPSGERSRLA | |
| | | | | | RGSTIRSTFHGGQVRDRRAGGGGGGGVQNGPP | |
| | | | | | ASPTLAHEAAPLPAGRPRPTTNLFTKLTSKLTRRV | |
| | | | | | TLDPSKRQNSNRCVSGASLPQGSKIRSQTNLRES | |
| | | | | | GDLRSQVAIYLGIKRKPPPGCSDSPGVNPAFLYK | |
| | | | | | VVKASS | |
| 382 | JHU1 | NIV | 1.10370141 | MARK4 | MSSRTVLAPGNDRNSDTHGTLGSGRSSDKGPS | Core Human ORF Only |
| | 7311 | O_T | 9 | | WSSRSLGARCRNSIASCPEEQPHVGNYRLLRTIG | |
| | | OX | | | KGNFAKVKLARHILTGREVAIKIIDKTQLNPSSLQK | |
| | | | | | LFREVRIMKGLNHPNIVKLFEVIETEKTLYLVMEYA | |
| | | | | | SAGEVFDYLVSHGRMKEKEARAKFRQIVSAVHYC | |
| | | | | | HQKNIVHRDLKAENLLLDAEANIKIADFGFSNEFTL | |
| | | | | | GSKLDTFCGSPPYAAPELFQGKKYDGPEVDIWSL | |
| | | | | | GVILYTLVSGSLPFDGHNLKELRERVLRGKYRVPF | |
| | | | | | YMSTDCESILRRFLVLNPAKRCTLEQIMKDKWINI | |
| | | | | | GYEGEELKPYTEPEEDFGDTKRIEVMVGMGYTR | |
| | | | | | EEIKESLTSQKYNEVTATYLLLGRKTEEGGDRGA | |
| | | | | | PGLALARVRAPSDTTNGTSSSKGTSHSKGQRSS | |
| | | | | | SSTYHRQRRHSDFCGPSPAPLHPKRSPTSTGEA | |
| | | | | | ELKEERLPGRKASCSTAGSGSRGLPPSSPMVSS | |
| | | | | | AHNPNKAEIPERRKDSTSTPNNLPPSMMTRRNTY | |
| | | | | | VCTERPGAERPSLLPNGKENSSGTPRVPPASPS | |
| | | | | | SHSLAPPSGERSRLARGSTIRSTFHGGQVRDRR | |
| | | | | | AGGGGGGGVQNGPPASPTLAHEAAPLPAGRPR | |
| | | | | | PTTNLFTKLTSKLTRRVTLDPSKRQNSNRCVSGA | |
| | | | | | SLPQGSKIRSQTNLRESGDLRSQVAIYLGIKRKPP | |
| | | | | | PGCSDSPGV | |
| 383 | JHU1 | NIV | 1.12832733 | SV2A | MSPILGYWKIKGLVQPTRLLLEYLEEKYEEHLYER | Full Literal Sequence- |
| | 7390 | O_T | 5 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEEGFRDRAAFIRGA | |
| | | | | | KDIAKEVKKHAAKKVVKGLDRVQDEYSRRSYSRF | |
| | | | | | EEEDDDDDFPAPSDGYYRGEGTQDEEEGGASS | |
| | | | | | DATEGHDEDDEIYEGEYQDIPRAESGGKGERMA | |
| | | | | | DGAPLAGVRGGLSDGEGPPGGRGEAQRRKERE | |
| | | | | | ELAQQYEAILRECGHGRFQWTLYFVLGLALMADG | |
| | | | | | VEVFVVGFVLPSAEKDMCLSDSNKGMLGLIVYLG | |
| | | | | | MMVGAFLWGGLADRLGRRQCLLISLSVNSVFAFF | |
| | | | | | SSFVQGYGTFLFCRLLSGVGIGGSIPIVFSYFSEFL | |
| | | | | | AQEKRGEHLSWLCMFWMIGGVYAAAMAWAIIPH | |
| | | | | | YGWSFQMGSAYQFHSWRVFVLVCAFPSVFAIGA | |
| | | | | | LTTQPESPRFFLENGKHDEAWMVLKQVHDTNMR | |
| | | | | | AKGHPERVFSVTHIKTIHQEDELIEIQSDTGTWYQ | |
| | | | | | RWGVRALSLGGQVWGNFLSCFGPEYRRITLMM | |
| | | | | | MGVWFTMSFSYYGLTVWFPDMIRHLQAVDCASR | |
| | | | | | TKVFPGERVGHVTFNFTLENQIHRGGQYFNDKFI | |
| | | | | | GLRLKSVSFEDSLFEECYFEDVTSSNTFFRNCTFI | |
| | | | | | NTVFYNTDLFEYKFVNSRLINSTFLHNKEGCPLDV | |
| | | | | | TGTGEGAYMVYFVSFLGTLAVLPGNIVSALLMDKI | |
| | | | | | GRLRMLAGSSVMSCVSCFFLSFGNSESAMIALLC | |
| | | | | | LFGGVSIASWNALDVLTVELYPSDKRTTAFGFLNA | |
| | | | | | LCKLAAVLGISIFTSFVGITKAAPILFASAALALGS | |
| | | | | | SLALKLPETRGQVLQNPAFLYKVVKASS | |
| 384 | JHU1 | NIV | 1.12832733 | SV2A | MEEGFRDRAAFIRGAKDIAKEVKKHAAKKVVKGL | Core Human ORF Only |
| | 7390 | O_T | 5 | | DRVQDEYSRRSYSRFEEEDDDDDFPAPSDGYYR | |
| | | OX | | | GEGTQDEEEGGASSDATEGHDEDDEIYEGEYQD | |
| | | | | | IPRAESGGKGERMADGAPLAGVRGGLSDGEGPP | |
| | | | | | GGRGEAQRRKEREELAQQYEAILRECGHGRFQ | |
| | | | | | WTLYFVLGLALMADGVEVFVVGFVLPSAEKDMCL | |
| | | | | | SDSNKGMLGLIVYLGMMVGAFLWGGLADRLGRR | |
| | | | | | QCLLISLSVNSVFAFFSSFVQGYGTFLFCRLLSGV | |
| | | | | | GIGGSIPIVFSYFSEFLAQEKRGEHLSWLCMFWMI | |
| | | | | | GGVYAAAMAWAIIPHYGWSFQMGSAYQFHSWR | |
| | | | | | VFVLVCAFPSVFAIGALTTQPESPRFFLENGKHDE | |
| | | | | | AWMVLKQVHDTNMRAKGHPERVFSVTHIKTIHQE | |
| | | | | | DELIEIQSDTGTWYQRWGVRALSLGGQVWGNFL | |
| | | | | | SCFGPEYRRITLMMMGVWFTMSFSYYGLTVWFP | |
| | | | | | DMIRHLQAVDCASRTKVFPGERVGHVTFNFTLEN | |
| | | | | | QIHRGGQYFNDKFIGLRLKSVSFEDSLFEECYFED | |
| | | | | | VTSSNTFFRNCTFINTVFYNTDLFEYKFVNSRLINS | |
| | | | | | TFLHNKEGCPLDVTGTGEGAYMVYFVSFLGTLAV | |
| | | | | | LPGNIVSALLMDKIGRLRMLAGSSVMSCVSCFFLS | |
| | | | | | FGNSESAMIALLCLFGGVSIASWNALDVLTVELYP | |
| | | | | | SDKRTTAFGFLNALCKLAAVLGISIFTSFVGITKAA | |
| | | | | | PILFASAALALGSSLALKLPETRGQVLQ | |
| 385 | JHU1 | NIV | 0.4703004 | TGFBR | MSPILGYWKIKGLVQPTRLLLEYLEEKYEEHLYER | Full Literal Sequence- |
| | 8329 | O_T | 9 | AP1 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMSIKAFTLVSAVER | |
| | | | | | ELLMGDKERVNIECVECCGRDLYVGTNDCFVYHF | |
| | | | | | LLEERPVPAGPATFTATKQLQRHLGFKKPVNELR | |
| | | | | | AASALNRLLVLCDNSISLVNMLNLEPVPSGARIKG | |
| | | | | | AATFALNENPVSGDPFCVEVCIISVKRRTIQMFLV | |
| | | | | | YEDRVQIVKEVSTAEQPLAVAVDGHFLCLALTTQ | |
| | | | | | YIIHNYSTGVSQDLFPYCSEERPPIVKRIGRQEFLL | |
| | | | | | AGPGGLGMFATVAGISQRAPVHWSENVIGAAVS | |
| | | | | | FPYVIALDDEFITVHSMLDQQQKQTLPFKEGHILQ | |
| | | | | | DFEGRVIVATSKGVYILVPLPLEKQIQDLLASRRVE | |
| | | | | | EALVLAKGARRNIPKEKFQVMYRRILQQAGFIQFA | |
| | | | | | QLQFLEAKELFRSGQLDVRELISLYPFLLPTSSSF | |
| | | | | | TRSHPPLHEYADLNQLTQGDQEKMAKCKRFLMS | |
| | | | | | YLNEVRSTEVANGYKEDIDTALLKLYAEADHDSLL | |
| | | | | | DLLVTENFCLLTDSAAWLEKHKKYFALGLLYHYN | |
| | | | | | NQDAAAVQLWVNIVNGDVQDSTRSDLYEYIVDFL | |
| | | | | | TYCLDEELVWAYADVWLQKSEEVGVQVFTKRPL | |
| | | | | | DEQQKNSFNPDDIINCLKKYPKALVKYLEHLVIDK | |
| | | | | | RLQKEEYHTHLAVLYLEEVLLQRASASGKGAEAT | |
| | | | | | ETQAKLRRLLQKSDLYRVHFLLERLQGAGLPMES | |
| | | | | | AILHGKLGEHEKALHILVHELQDFAAAEDYCLWCS | |
| | | | | | EGRDPPHRQQLFHTLLAIYLHAGPTAHELAVAAV | |
| | | | | | DLLNRHATEFDAAQVLQMLPDTWSVQLLCPFLM | |
| | | | | | GAMRDSIHARRTMQVALGLARSENLIYTYDKMKL | |
| | | | | | KGSSIQLSDKKLCQICQNPFCEPVFVRYPNGGLV | |
| | | | | | HTHCAASRHTNPSSSSPGTRTNPAFLYKVVKASS | |
| 386 | JHU1 | NIV | 0.4703004 | TGFBR | MMSIKAFTLVSAVERELLMGDKERVNIECVECCG | Core Human ORF Only |
| | 8329 | O_T | 9 | AP1 | RDLYVGTNDCFVYHFLLEERPVPAGPATFTATKQ | |
| | | OX | | | LQRHLGFKKPVNELRAASALNRLLVLCDNSISLVN | |
| | | | | | MLNLEPVPSGARIKGAATFALNENPVSGDPFCVE | |
| | | | | | VCIISVKRRTIQMFLVYEDRVQIVKEVSTAEQPLAV | |
| | | | | | AVDGHFLCLALTTQYIIHNYSTGVSQDLFPYCSEE | |
| | | | | | RPPIVKRIGRQEFLLAGPGGLGMFATVAGISQRAP | |
| | | | | | VHWSENVIGAAVSFPYVIALDDEFITVHSMLDQQ | |
| | | | | | QKQTLPFKEGHILQDFEGRVIVATSKGVYILVPLPL | |
| | | | | | EKQIQDLLASRRVEEALVLAKGARRNIPKEKFQV | |
| | | | | | MYRRILQQAGFIQFAQLQFLEAKELFRSGQLDVR | |
| | | | | | ELISLYPFLLPTSSSFTRSHPPLHEYADLNQLTQG | |
| | | | | | DQEKMAKCKRFLMSYLNEVRSTEVANGYKEDIDT | |
| | | | | | ALLKLYAEADHDSLLDLLVTENFCLLTDSAAWLEK | |
| | | | | | HKKYFALGLLYHYNNQDAAAVQLWVNIVNGDVQ | |
| | | | | | DSTRSDLYEYIVDFLTYCLDEELVWAYADVWLQK | |
| | | | | | SEEVGVQVFTKRPLDEQQKNSFNPDDIINCLKKY | |
| | | | | | PKALVKYLEHLVIDKRLQKEEYHTHLAVLYLEEVLL | |
| | | | | | QRASASGKGAEATETQAKLRRLLQKSDLYRVHFL | |
| | | | | | LERLQGAGLPMESAILHGKLGEHEKALHILVHELQ | |
| | | | | | DFAAAEDYCLWCSEGRDPPHRQQLFHTLLAIYLH | |
| | | | | | AGPTAHELAVAAVDLLNRHATEFDAAQVLQMLPD | |
| | | | | | TWSVQLLCPFLMGAMRDSIHARRTMQVALGLAR | |
| | | | | | SENLIYTYDKMKLKGSSIQLSDKKLCQICQNPFCE | |
| | | | | | PVFVRYPNGGLVHTHCAASRHTNPSSSSPGTRT | |
| 387 | JHU1 | NIV | 1.30458492 | DEFB1 | MSPILGYWKIKGLVQPTRLLLEYLEEKYEEHLYER | Full Literal Sequence- |
| | 8757 | O_T | 5 | 09P1 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMRLHLLLLILLLFSILL | |
| | | | | | SPVRGGLGPAEGHCLNLFGVCRTDVCNIVEDQIG | |
| | | | | | ACRRRMKCCRAWWILMPIPTPLIMSDYQEPLKPN | |
| | | | | | LKNPAFLYKVVKASS | |
| 388 | JHU1 | NIV | 1.30458492 | DEFB1 | MRLHLLLLILLLFSILLSPVRGGLGPAEGHCLNLFG | Core Human ORF Only |
| | 8757 | O_T | 5 | 09P1 | VCRTDVCNIVEDQIGACRRRMKCCRAVWVILMPIP | |
| | | OX | | | TPLIMSDYQEPLKPNLK | |
| 389 | JHU1 | NIV | 2.9856812 | SPANX | MSPILGYWKIKGLVQPTRLLLEYLEEKYEEHLYER | Full Literal Sequence- |
| | 8818 | O_T | 8 | A1 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDKQSSAGGVKRSV | |
| | | | | | PCDSNEANEMMPETPTGDSDPQPAPKKMKTSES | |
| | | | | | STILVVRYRRNFKRTSPEELLNDHARENRINPLQM | |
| | | | | | EEEEFMEIMVEIPAKNPAFLYKVVKASS | |
| 390 | JHU1 | NIV | 2.9856812 | SPANX | MDKQSSAGGVKRSVPCDSNEANEMMPETPTGD | Core Human ORF Only |
| | 8818 | O_T | 8 | A1 | SDPQPAPKKMKTSESSTILVVRYRRNFKRTSPEE | |
| | | OX | | | LLNDHARENRINPLQMEEEEFMEIMVEIPAK | |
| 391 | JHU1 | NIV | 1.3667763 | CYP11 | MSPILGYWKIKGLVQPTRLLLEYLEEKYEEHLYER | Full Literal Sequence- |
| | 8997 | O_T | 5 | B2 | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMALRAKAEVCVAAP | |
| | | | | | WLSLQRARALGTRAARAPRTVLPFEAMPQHPGN | |
| | | | | | RWLRLLQIWREQGYEHLHLEMHQTFQELGPIFRY | |
| | | | | | NLGGPRMVCVMLPEDVEKLQQVDSLHPCRMILE | |
| | | | | | PVWAYRQHRGHKCGVFLLNGPEWRFNRLRLNP | |
| | | | | | DVLSPKAVQRFLPMVDAVARDFSQALKKKVLQNA | |
| | | | | | RGSLTLDVQPSIFHYTIEASNLALFGERLGLVGHS | |
| | | | | | PSSASLNFLHALEVMFKSTVQLMFMPRSLSRWIS | |
| | | | | | PKVWKEHFEAWDCIFQYGDNCIQKIYQELAFNRP | |
| | | | | | QHYTGIVAELLLKAELSLEAIKANSMELTAGSVDT | |
| | | | | | TAFPLLMTLFELARNPDVQQILRQESLAAAASISE | |
| | | | | | HPQKATTELPLLRAALKETLRLYPVGLFLERVVSS | |
| | | | | | DLVLQNYHIPAGTLVQVFLYSLGRNAALFPRPERY | |
| | | | | | NPQRWLDIRGSGRNFHHVPFGFGMRQCLGRRL | |
| | | | | | AEAEMLLLLHHVLKHFLVETLTQEDIKMVYSFILRP | |
| | | | | | GTSPLLTFRAIN | |
| 392 | JHU1 | NIV | 1.3667763 | CYP11 | MALRAKAEVCVAAPWLSLQRARALGTRAARAPR | Core Human ORF Only |
| | 8997 | O_T | 5 | B2 | TVLPFEAMPQHPGNRWLRLLQIWREQGYEHLHL | |
| | | OX | | | EMHQTFQELGPIFRYNLGGPRMVCVMLPEDVEK | |
| | | | | | LQQVDSLHPCRMILEPWVAYRQHRGHKCGVFLL | |
| | | | | | NGPEWRFNRLRLNPDVLSPKAVQRFLPMVDAVA | |
| | | | | | RDFSQALKKKVLQNARGSLTLDVQPSIFHYTIEAS | |
| | | | | | NLALFGERLGLVGHSPSSASLNFLHALEVMFKST | |
| | | | | | VQLMFMPRSLSRWISPKVWKEHFEAWDCIFQYG | |
| | | | | | DNCIQKIYQELAFNRPQHYTGIVAELLLKAELSLEA | |
| | | | | | IKANSMELTAGSVDTTAFPLLMTLFELARNPDVQ | |
| | | | | | QILRQESLAAAASISEHPQKATTELPLLRAALKETL | |
| | | | | | RLYPVGLFLERVVSSDLVLQNYHIPAGTLVQVFLY | |
| | | | | | SLGRNAALFPRPERYNPQRWLDIRGSGRNFHHV | |
| | | | | | PFGFGMRQCLGRRLAEAEMLLLLHHVLKHFLVET | |
| | | | | | LTQEDIKMVYSFILRPGTSPLLTFRAIN | |
| 393 | JHU1 | NIV | 1.88111107 | PIGR | MSPILGYWKIKGLVQPTRLLLEYLEEKYEEHLYER | Full Literal Sequence- |
| | 9125 | O_T | 2 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLLFVLTCLLAVFPAI | |
| | | | | | STKSPIFGPEEVNSVEGNSVSITCYYPPTSVNRHT | |
| | | | | | RKYWCRQGARGGCITLISSEGYVSSKYAGRANLT | |
| | | | | | NFPENGTFVVNIAQLSQDDSGRYKCGLGINSRGL | |
| | | | | | SFDVSLEVSQGPGLLNDTKVYTVDLGRTVTINCP | |
| | | | | | FKTENAQKRKSLYKQIGLYPVLVIDSSGYVNPNYT | |
| | | | | | GRIRLDIQGTGQLLFSVVINQLRLSDAGQYLCQAG | |
| | | | | | DDSNSNKKNADLQVLKPEPELVYEDLRGSVTFHC | |
| | | | | | ALGPEVANVAKFLCRQSSGENCDVVVNTLGKRA | |
| | | | | | PAFEGRILLNPQDKDGSFSVVITGLRKEDAGRYLC | |
| | | | | | GAHSDGQLQEGSPIQAWQLFVNEESTIPRSPTVV | |
| | | | | | KGVAGGSVAVLCPYNRKESKSIKYWCLWEGAQN | |
| | | | | | GRCPLLVDSEGVWKAQYEGRLSLLEEPGNGTFT | |
| | | | | | VILNQLTSRDAGFYWCLTNGDTLWRTTVEIKIIEG | |
| | | | | | EPNLKVPGNVTAVLGETLKVPCHFPCKFSSYEKY | |
| | | | | | WCKWNNTGCQALPSQDEGPSKAFVNCDENSRL | |
| | | | | | VSLTLNLVTRADEGWYWCGVKQGHFYGETAAVY | |
| | | | | | VAVEERKAAGSRDVSLAKADAAPDEKVLDSGFR | |
| | | | | | EIENKAIQDPRLFAEEKAVADTRDQADGSRASVD | |
| | | | | | SGSSEEQGGSSRALVSTLVPLGLVLAVGAVAVGV | |
| | | | | | ARARHRKNVDRVSIRSYRTDISMSDFENSREFGA | |
| | | | | | NDNMGASSITQETSLGGKEEFVATTESTTETKEP | |
| | | | | | KKAKRSSKEEAEMAYKDFLLQSSTVAAEAQDGP | |
| | | | | | QEA | |
| 394 | JHU1 | NIV | 1.88111107 | PIGR | MLLFVLTCLLAVFPAISTKSPIFGPEEVNSVEGNSV | Core Human ORF Only |
| | 9125 | O_T | 2 | | SITCYYPPTSVNRHTRKYWCRQGARGGCITLISS | |
| | | OX | | | EGYVSSKYAGRANLTNFPENGTFVVNIAQLSQDD | |
| | | | | | SGRYKCGLGINSRGLSFDVSLEVSQGPGLLNDTK | |
| | | | | | VYTVDLGRTVTINCPFKTENAQKRKSLYKQIGLYP | |
| | | | | | VLVIDSSGYVNPNYTGRIRLDIQGTGQLLFSVVIN | |
| | | | | | QLRLSDAGQYLCQAGDDSNSNKKNADLQVLKPE | |
| | | | | | PELVYEDLRGSVTFHCALGPEVANVAKFLCRQSS | |
| | | | | | GENCDVVVNTLGKRAPAFEGRILLNPQDKDGSFS | |
| | | | | | VVITGLRKEDAGRYLCGAHSDGQLQEGSPIQAW | |
| | | | | | QLFVNEESTIPRSPTVVKGVAGGSVAVLCPYNRK | |
| | | | | | ESKSIKYWCLWEGAQNGRCPLLVDSEGVWKAQY | |
| | | | | | EGRLSLLEEPGNGTFTVILNQLTSRDAGFYWCLT | |
| | | | | | NGDTLWRTTVEIKIIEGEPNLKVPGNVTAVLGETL | |
| | | | | | KVPCHFPCKFSSYEKYWCKWNNTGCQALPSQD | |
| | | | | | EGPSKAFVNCDENSRLVSLTLNLVTRADEGWYW | |
| | | | | | CGVKQGHFYGETAAVYVAVEERKAAGSRDVSLA | |
| | | | | | KADAAPDEKVLDSGFREIENKAIQDPRLFAEEKAV | |
| | | | | | ADTRDQADGSRASVDSGSSEEQGGSSRALVSTL | |
| | | | | | VPLGLVLAVGAVAVGVARARHRKNVDRVSIRSYR | |
| | | | | | TDISMSDFENSREFGANDNMGASSITQETSLGGK | |
| | | | | | EEFVATTESTTETKEPKKAKRSSKEEAEMAYKDF | |
| | | | | | LLQSSTVAAEAQDGPQEA | |
| 395 | JHU1 | NIV | 0.41786567 | MMP11 | MSPILGYWKIKGLVQPTRLLLEYLEEKYEEHLYER | Full Literal Sequence- |
| | 9215 | O_T | 9 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAPAAWLRSAAARA | |
| | | | | | LLPPMLLLLLQPPPLLARALPPDAHHLHAERRGP | |
| | | | | | QPWHAALPSSPAPAPATQEAPRPASSLRPPRCG | |
| | | | | | VPDPSDGLSARNRQKRFVLSGGRWEKTDLTYRIL | |
| | | | | | RFPWQLVQEQVRQTMAEALKVWSDVTPLTFTEV | |
| | | | | | HEGRADIMIDFARYWHGDDLPFDGPGGILAHAFF | |
| | | | | | PKTHREGDVHFDYDETWTIGDDQGTDLLQVAAH | |
| | | | | | EFGHVLGLQHTTAAKALMSAFYTFRYPLSLSPDD | |
| | | | | | CRGVQHLYGQPWPTVTSRTPALGPQAGIDTNEIA | |
| | | | | | PLEPDAPPDACEASFDAVSTIRGELFFFKAGFVW | |
| | | | | | RLRGGQLQPGYPALASRHWQGLPSPVDAAFEDA | |
| | | | | | QGHIWFFQGAQYVWYDGEKPVLGPAPLTELGLV | |
| | | | | | RFPVHAALVWGPEKNKIYFFRGRDYWRFHPSTR | |
| | | | | | RVDSPVPRRATDWRGVPSEIDAAFQDADGYAYF | |
| | | | | | LRGRLYWKFDPVKVKALEGFPRLVGPDFFGCAE | |
| | | | | | PANTFL | |
| 396 | JHU1 | NIV | 0.41786567 | MMP11 | MAPAAWLRSAAARALLPPMLLLLLQPPPLLARAL | Core Human ORF Only |
| | 9215 | O_T | 9 | | PPDAHHLHAERRGPQPWHAALPSSPAPAPATQE | |
| | | OX | | | APRPASSLRPPRCGVPDPSDGLSARNRQKRFVL | |
| | | | | | SGGRWEKTDLTYRILRFPWQLVQEQVRQTMAEA | |
| | | | | | LKVWSDVTPLTFTEVHEGRADIMIDFARYWHGDD | |
| | | | | | LPFDGPGGILAHAFFPKTHREGDVHFDYDETWTI | |
| | | | | | GDDQGTDLLQVAAHEFGHVLGLQHTTAAKALMS | |
| | | | | | AFYTFRYPLSLSPDDCRGVQHLYGQPWPTVTSR | |
| | | | | | TPALGPQAGIDTNEIAPLEPDAPPDACEASFDAVS | |
| | | | | | TIRGELFFFKAGFVWRLRGGQLQPGYPALASRH | |
| | | | | | WQGLPSPVDAAFEDAQGHIWFFQGAQYWVYDG | |
| | | | | | EKPVLGPAPLTELGLVRFPVHAALVWGPEKNKIY | |
| | | | | | FFRGRDYWRFHPSTRRVDSPVPRRATDWRGVP | |
| | | | | | SEIDAAFQDADGYAYFLRGRLYWKFDPVKVKALE | |
| | | | | | GFPRLVGPDFFGCAEPANTFL | |
| 397 | JHU1 | NIV | 0.41995507 | Nol3 | MSPILGYWKIKGLVQPTRLLLEYLEEKYEEHLYER | Full Literal Sequence- |
| | 9589 | O_T | 5 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGNVQERPSETIDR | |
| | | | | | ERKRLVETLQADSGLLLDALVARGVLTGPEYEAL | |
| | | | | | DALPDAERRVRRLLLLVQSKGEAACQELLRCAQQ | |
| | | | | | TVRMPDPAWDWQHVGPGYRNRSYDPSCPGHW | |
| | | | | | TPEAPSSGTTCPELPRASEQEEVGGPEGSEALQ | |
| | | | | | PRTPEEPELEAEATEGDEPDLEQEMNPEQEPEP | |
| | | | | | EPEPEPEPEPEPEPEPEPEPEPEPEPEPEPDFQE | |
| | | | | | EDEFEDSNPAFLYKVVKASS | |
| 398 | JHU1 | NIV | 0.41995507 | Nol3 | MGNVQERPSETIDRERKRLVETLQADSGLLLDAL | Core Human ORF Only |
| | 9589 | O_T | 5 | | VARGVLTGPEYEALDALPDAERRVRRLLLLVQSK | |
| | | OX | | | GEAACQELLRCAQQTVRMPDPAWDWQHVGPGY | |
| | | | | | RNRSYDPSCPGHWTPEAPSSGTTCPELPRASEQ | |
| | | | | | EEVGGPEGSEALQPRTPEEPELEAEATEGDEPDL | |
| | | | | | EQEMNPEQEPEPEPEPEPEPEPEPEPEPEPEPE | |
| | | | | | PEPEPEPEPDFQEEDEFEDS | |
| 399 | JHU1 | NIV | 1.44779559 | ECD | MSPILGYWKIKGLVQPTRLLLEYLEEKYEEHLYER | Full Literal Sequence- |
| | 9814 | O_T | 9 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEETMKLATMEDTV | |
| | | | | | EYCLFLIPDESRDSDKHKEILQKYIERIITRFAPMLV | |
| | | | | | PYIWQNQPFNLKYKPGKGGVPAHMFGVTKFGDN | |
| | | | | | IEDEWFIVYVIKQITKEFPELVARIEDNDGEFLLIEA | |
| | | | | | ADFLPKWLDPENSTNRVFFCHGELCIIPAPRKSG | |
| | | | | | AESWLPTTPPTIPQALNIITAHSEKILASESIRAAVN | |
| | | | | | RRIRGYPEKIQASLHRAHCFLPAGIVAVLKQRPRL | |
| | | | | | VAAAVQAFYLRDPIDLRACRVFKTFLPETRIMTSV | |
| | | | | | TFTKCLYAQLVQQRFVPDRRSGYRLPPPSDPQY | |
| | | | | | RAHELGMKLAHGFEILCSKCSPHFSDCKKSLVTA | |
| | | | | | SPLWASFLESLKKNDYFKGLIEGSAQYRERLEMA | |
| | | | | | ENYFQLSVDWPESSLAMSPGEEILTLLQTIPFDIE | |
| | | | | | DLKKEAANLPPEDDDQWLDLSPDQLDQLLQEAV | |
| | | | | | GKKESESVSKEEKEQNYDLTEVSESMKAFISKVS | |
| | | | | | THKGAELPREPSEAPITFDADSFLNYFDKILGPRP | |
| | | | | | NESDSDDLDDEDFECLDSDDDLDFETHEPGEEA | |
| | | | | | SLKGTLDNLKSYMAQMDQELAHTCISKSFTTRNQ | |
| | | | | | VEPVSQTTDNNSDEEDSGTGESVMAPVDVDLNL | |
| | | | | | VSNILESYSSQAGLAGPASNLLQSMGVQLPDNTD | |
| | | | | | HRPTSKPTKNNPAFLYKVVKASS | |
| 400 | JHU1 | NIV | 1.44779559 | ECD | MEETMKLATMEDTVEYCLFLIPDESRDSDKHKEIL | Core Human ORF Only |
| | 9814 | O_T | 9 | | QKYIERIITRFAPMLVPYIWQNQPFNLKYKPGKGG | |
| | | OX | | | VPAHMFGVTKFGDNIEDEWFIVYVIKQITKEFPEL | |
| | | | | | VARIEDNDGEFLLIEAADFLPKWLDPENSTNRVFF | |
| | | | | | CHGELCHPAPRKSGAESWLPTTPPTIPQALNIITA | |
| | | | | | HSEKILASESIRAAVNRRIRGYPEKIQASLHRAHCF | |
| | | | | | LPAGIVAVLKQRPRLVAAAVQAFYLRDPIDLRACR | |
| | | | | | VFKTFLPETRIMTSVTFTKCLYAQLVQQRFVPDRR | |
| | | | | | SGYRLPPPSDPQYRAHELGMKLAHGFEILCSKCS | |
| | | | | | PHFSDCKKSLVTASPLWASFLESLKKNDYFKGLIE | |
| | | | | | GSAQYRERLEMAENYFQLSVDWPESSLAMSPGE | |
| | | | | | EILTLLQTIPFDIEDLKKEAANLPPEDDDQWLDLSP | |
| | | | | | DQLDQLLQEAVGKKESESVSKEEKEQNYDLTEVS | |
| | | | | | ESMKAFISKVSTHKGAELPREPSEAPITFDADSFL | |
| | | | | | NYFDKILGPRPNESDSDDLDDEDFECLDSDDDLD | |
| | | | | | FETHEPGEEASLKGTLDNLKSYMAQMDQELAHT | |
| | | | | | CISKSFTTRNQVEPVSQTTDNNSDEEDSGTGESV | |
| | | | | | MAPVDVDLNLVSNILESYSSQAGLAGPASNLLQS | |
| | | | | | MGVQLPDNTDHRPTSKPTKN | |
| 401 | JHU3 | NIV | 0.58816702 | RASEF | MSPILGYWKIKGLVQPTRLLLEYLEEKYEEHLYER | Full Literal Sequence- |
| | 0349 | O_T | 8 | | DEGDKWRNKKFELGLEFPNLPYYIDGDVKLTQSM | (including GST-tag, 6- |
| | | OX | | | AIIRYIADKHNMLGGCPKERAEISMLEGAVLDIRYG | His leader, cloning |
| | | | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEADGDGEELARLR | |
| | | | | | SVFAACDANRSGRLEREEFRALCTELRVRPADAE | |
| | | | | | AVFQRLDADRDGAITFQEFARGFLGSLRGGRRR | |
| | | | | | DWGPLDPAPAVSEAGPETHDSEEDEGDEDAAAA | |
| | | | | | LATSCGPASPGRAWQDFQARLGDEAKFIPREEQ | |
| | | | | | VSTLYQNINLVEPRLIQPYEHVIKNFIREIRLQSTE | |
| | | | | | MENLAIAVKRAQDKAAMQLSELEEEMDQRIQAAE | |
| | | | | | HKTRKDEKRKAEEALSDLRRQYETEVGDLQVTIK | |
| | | | | | KLRKLEEQSKRVSQKEDVAALKKQIYDLSMENQK | |
| | | | | | VKKDLLEAQTNIAFLQSELDALKSDYADQSLNTER | |
| | | | | | DLEIIRAYTEDRNSLERQIEILQTANRKLHDSNDGL | |
| | | | | | RSALENSYSKFNRSLHINNISPGNTISRSSPKFIGH | |
| | | | | | SPQPLGYDRSSRSSYVDEDCDSLALCDPLQRTN | |
| | | | | | CEVDSLPESCFDSGLSTLRDPNEYDSEVEYKHQ | |
| | | | | | RGFQRSHGVQESFGGDASDTDVPDIRDEETFGL | |
| | | | | | EDVASVLDWKPQGSVSEGSIVSSSRKPISALSPQ | |
| | | | | | TDLVDDNAKSFSSQKAYKIVLAGDAAVGKSSFLM | |
| | | | | | RLCKNEFRENISATLGVDFQMKTLIVDGERTVLQL | |
| | | | | | WDTAGQERFRSIAKSYFRKADGVLLLYDVTCEKS | |
| | | | | | FLNIREVWDMIEDAAHETVPIMLVGNKADIRDTAA | |
| | | | | | TEGQKCVPGHFGEKLAMTYGALFCETSAKDGSNI | |
| | | | | | VEAVLHLAREVKKRTDKDDSRSITNLTGTNSKKS | |
| | | | | | PQMKNCCNG | |
| 402 | JHU3 | NIV | 0.58816702 | RASEF | MEADGDGEELARLRSVFAACDANRSGRLEREEF | Core Human ORF Only |
| | 0349 | O_T | 8 | | RALCTELRVRPADAEAVFQRLDADRDGAITFQEF | |
| | | OX | | | ARGFLGSLRGGRRRDWGPLDPAPAVSEAGPETH | |
| | | | | | DSEEDEGDEDAAAALATSCGPASPGRAWQDFQ | |
| | | | | | ARLGDEAKFIPREEQVSTLYQNINLVEPRLIQPYE | |
| | | | | | HVIKNFIREIRLQSTEMENLAIAVKRAQDKAAMQL | |
| | | | | | SELEEEMDQRIQAAEHKTRKDEKRKAEEALSDLR | |
| | | | | | RQYETEVGDLQVTIKKLRKLEEQSKRVSQKEDVA | |
| | | | | | ALKKQIYDLSMENQKVKKDLLEAQTNIAFLQSELD | |
| | | | | | ALKSDYADQSLNTERDLEIIRAYTEDRNSLERQIEI | |
| | | | | | LQTANRKLHDSNDGLRSALENSYSKFNRSLHINNI | |
| | | | | | SPGNTISRSSPKFIGHSPQPLGYDRSSRSSYVDE | |
| | | | | | DCDSLALCDPLQRTNCEVDSLPESCFDSGLSTLR | |
| | | | | | DPNEYDSEVEYKHQRGFQRSHGVQESFGGDAS | |
| | | | | | DTDVPDIRDEETFGLEDVASVLDWKPQGSVSEG | |
| | | | | | SIVSSSRKPISALSPQTDLVDDNAKSFSSQKAYKI | |
| | | | | | VLAGDAAVGKSSFLMRLCKNEFRENISATLGVDF | |
| | | | | | QMKTLIVDGERTVLQLWDTAGQERFRSIAKSYFR | |
| | | | | | KADGVLLLYDVTCEKSFLNIREVWDMIEDAAHETV | |
| | | | | | PIMLVGNKADIRDTAATEGQKCVPGHFGEKLAMT | |
| | | | | | YGALFCETSAKDGSNIVEAVLHLAREVKKRTDKD | |
| | | | | | DSRSITNLTGTNSKKSPQMKNCCNG | |
| 403 | JHUO | NIV | 0.73038632 | SRPX2 | MSPILGYWKIKGLVQPTRLLLEYLEEKYEEHLYER | Full Literal Sequence- |
| | 0749 | OIPI | 8 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASQLTQRGALFLLF | |
| | | | | | FLTPAVTPTWYAGSGYYPDESYNEVYAEEVPQA | |
| | | | | | PALDYRVPRWCYTLNIQDGEATCYSPKGGNYHS | |
| | | | | | SLGTRCELSCDRGFRLIGRRSVQCLPSRRWSGT | |
| | | | | | AYCRQMRCHALPFITSGTYTCTNGVLLDSRCDYS | |
| | | | | | CSSGYHLEGDRSRICMEDGRWSGGEPVCVDIDP | |
| | | | | | PKIRCPHSREKMAEPEKLTARVYWDPPLVKDSAD | |
| | | | | | GTITRVTLRGPEPGSHFPEGEHVIRYTAYDRAYN | |
| | | | | | RASCKFIVKVQVRRCPTLKPPQHGYLTCTSAGDN | |
| | | | | | YGASCEYHCDGGYDRQGTPSRVCQSSRQWSGS | |
| | | | | | PPICAPMKINVNVNSAAGLLDQFYEKQRLLIISAPD | |
| | | | | | PSNRYYKMQISMLQQSTCGLDLRHVTIIELVGQPP | |
| | | | | | QEVGRIREQQLSANIIEELRQFQRLTRSYFNMVLI | |
| | | | | | DKQGIDRDRYMEPVTPEEIFTFIDDYLLSNQELTQ | |
| | | | | | RREQRDICE | |
| 404 | JHUO | NIV | 0.73038632 | SRPX2 | MASQLTQRGALFLLFFLTPAVTPTWYAGSGYYPD | Core Human ORF Only |
| | 0749 | OIPI | 8 | | ESYNEVYAEEVPQAPALDYRVPRWCYTLNIQDG | |
| | | _RF | | | EATCYSPKGGNYHSSLGTRCELSCDRGFRLIGRR | |
| | | S | | | SVQCLPSRRWSGTAYCRQMRCHALPFITSGTYT | |
| | | | | | CTNGVLLDSRCDYSCSSGYHLEGDRSRICMEDG | |
| | | | | | RWSGGEPVCVDIDPPKIRCPHSREKMAEPEKLTA | |
| | | | | | RVYWDPPLVKDSADGTITRVTLRGPEPGSHFPEG | |
| | | | | | EHVIRYTAYDRAYNRASCKFIVKVQVRRCPTLKPP | |
| | | | | | QHGYLTCTSAGDNYGASCEYHCDGGYDRQGTP | |
| | | | | | SRVCQSSRQWSGSPPICAPMKINVNVNSAAGLL | |
| | | | | | DQFYEKQRLLIISAPDPSNRYYKMQISMLQQSTC | |
| | | | | | GLDLRHVTIIELVGQPPQEVGRIREQQLSANIIEEL | |
| | | | | | RQFQRLTRSYFNMVLIDKQGIDRDRYMEPVTPEE | |
| | | | | | IFTFIDDYLLSNQELTQRREQRDICE | |
| 405 | JHUO | NIV | 0.61264371 | PANK1 | MSPILGYWKIKGLVQPTRLLLEYLEEKYEEHLYER | Full Literal Sequence- |
| | 1885 | OIPI | 2 | _frag | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAKGDSTNVDKLV | |
| | | | | | KDIYGGDYERFGLQGSAVASSFGNMMSKEKRDS | |
| | | | | | ISKEDLARATLVTITNNIGSIARMCALNENIDRVVF | |
| | | | | | VGNFLRINMVSMKLLAYAMDFWSKGQLKALFLEH | |
| | | | | | EGYFGAVGALLELFKMTDDK | |
| 406 | JHUO | NIV | 0.61264371 | PANK1 | MAAKGDSTNVDKLVKDIYGGDYERFGLQGSAVA | Core Human ORF Only |
| | 1885 | OIPI | 2 | _frag | SSFGNMMSKEKRDSISKEDLARATLVTITNNIGSIA | |
| | | _RF | | | RMCALNENIDRVVFVGNFLRINMVSMKLLAYAMD | |
| | | S | | | FWSKGQLKALFLEHEGYFGAVGALLELFKMTDDK | |
| 407 | JHUO | NIV | 0.47994368 | WNT7A | MSPILGYWKIKGLVQPTRLLLEYLEEKYEEHLYER | Full Literal Sequence- |
| | 2203 | OIPI | 5 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNRKARRCLGHLFL | |
| | | | | | SLGMVYLRIGGFSSVVALGASIICNKIPGLAPRQR | |
| | | | | | AICQSRPDAIIVIGEGSQMGLDECQFQFRNGRWN | |
| | | | | | CSALGERTVFGKELKVGSREAAFTYAIIAAGVAHA | |
| | | | | | ITAACTQGNLSDCGCDKEKQGQYHRDEGWKWG | |
| | | | | | GCSADIRYGIGFAKVFVDAREIKQNARTLMNLHN | |
| | | | | | NEAGRKILEENMKLECKCHGVSGSCTTKTCWTTL | |
| | | | | | PQFRELGYVLKDKYNEAVHVEPVRASRNKRPTFL | |
| | | | | | KIKKPLSYRKPMDTDLVYIEKSPNYCEEDPVTGSV | |
| | | | | | GTQGRACNKTAPQASGCDLMCCGRGYNTHQYA | |
| | | | | | RVWQCNCKFHWCCYVKCNTCSERTEMYTCK | |
| 408 | JHUO | NIV | 0.47994368 | WNT7A | MNRKARRCLGHLFLSLGMVYLRIGGFSSVVALGA | Core Human ORF Only |
| | 2203 | OIPI | 5 | | SIICNKIPGLAPRQRAICQSRPDAIIVIGEGSQMGL | |
| | | _RF | | | DECQFQFRNGRWNCSALGERTVFGKELKVGSR | |
| | | S | | | EAAFTYAIIAAGVAHAITAACTQGNLSDCGCDKEK | |
| | | | | | QGQYHRDEGWKWGGCSADIRYGIGFAKVFVDA | |
| | | | | | REIKQNARTLMNLHNNEAGRKILEENMKLECKCH | |
| | | | | | GVSGSCTTKTCWTTLPQFRELGYVLKDKYNEAV | |
| | | | | | HVEPVRASRNKRPTFLKIKKPLSYRKPMDTDLVYI | |
| | | | | | EKSPNYCEEDPVTGSVGTQGRACNKTAPQASGC | |
| | | | | | DLMCCGRGYNTHQYARVWQCNCKFHWCCYVK | |
| | | | | | CNTCSERTEMYTCK | |
| 409 | JHUO | NIV | 0.49951753 | DIDO1 | MSPILGYWKIKGLVQPTRLLLEYLEEKYEEHLYER | Full Literal Sequence- |
| | 2228 | OIPI | 8 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDDKGDPSNEEAPK | |
| | | | | | AIKPTSKEFRKTWGFRRTTIAKREGAGDAEADPL | |
| | | | | | EPPPPQQQLGLSLRRSGRQPKRTERVEQFLTIAR | |
| | | | | | RRGRRSMPVSLEDSGEPTSCPATDAETASEGSV | |
| | | | | | ESASETRSGPQSASTAVKERPASSEKVKGGDDH | |
| | | | | | DDTSDSDSDGLTLKELQNRLRRKREQEPTERPLK | |
| | | | | | GIQSRLRKKRREEGPAETVGSEASDTVEGVLPSK | |
| | | | | | QEPENDQGVVSQAGKDDRESKLEGKAAQDIKDE | |
| | | | | | EPGDLGRPKPECEGYDPNALYCICRQPHNNRFMI | |
| | | | | | CCDRCEEWFHGDCVGISEARGRLLERNGEDYIC | |
| | | | | | PNCTILQVQDETHSETADQQEAKWRPGDADGTD | |
| | | | | | CTSIGTIEQKSSEDQGIKGRIEKAANPSGKKKLKIF | |
| | | | | | QPVIEAPGASKCIGPGCCHVAQPDSVYCSNDCIL | |
| | | | | | KHAAATMKFLSSGKEQKPKPKEKMKMKPEKPSL | |
| | | | | | PKCGAQAGIKISSVHKRPAPEKKETTVKKAVVVPA | |
| | | | | | RSEALGKEAACESSTPSWASDHNYNAVKPEKTA | |
| | | | | | APSPSLLYKCSGKYLYSLHPSLIA | |
| 410 | JHUO | NIV | 0.49951753 | DIDO1 | MDDKGDPSNEEAPKAIKPTSKEFRKTWGFRRTTI | Core Human ORF Only |
| | 2228 | OIPI | 8 | | AKREGAGDAEADPLEPPPPQQQLGLSLRRSGRQ | |
| | | _RF | | | PKRTERVEQFLTIARRRGRRSMPVSLEDSGEPTS | |
| | | S | | | CPATDAETASEGSVESASETRSGPQSASTAVKE | |
| | | | | | RPASSEKVKGGDDHDDTSDSDSDGLTLKELQNR | |
| | | | | | LRRKREQEPTERPLKGIQSRLRKKRREEGPAETV | |
| | | | | | GSEASDTVEGVLPSKQEPENDQGVVSQAGKDDR | |
| | | | | | ESKLEGKAAQDIKDEEPGDLGRPKPECEGYDPN | |
| | | | | | ALYCICRQPHNNRFMICCDRCEEWFHGDCVGISE | |
| | | | | | ARGRLLERNGEDYICPNCTILQVQDETHSETADQ | |
| | | | | | QEAKWRPGDADGTDCTSIGTIEQKSSEDQGIKGR | |
| | | | | | IEKAANPSGKKKLKIFQPVIEAPGASKCIGPGCCH | |
| | | | | | VAQPDSVYCSNDCILKHAAATMKFLSSGKEQKPK | |
| | | | | | PKEKMKMKPEKPSLPKCGAQAGIKISSVHKRPAP | |
| | | | | | EKKETTVKKAVVVPARSEALGKEAACESSTPSWA | |
| | | | | | SDHNYNAVKPEKTAAPSPSLLYKCSGKYLYSLHP | |
| | | | | | SLIA | |
| 411 | JHUO | NIV | 0.5659150 | FAM15 | MSPILGYWKIKGLVQPTRLLLEYLEEKYEEHLYER | Full Literal Sequence- |
| | 2677 | OIPI | 5 | 6A | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMGLSNLSPGPGPS | |
| | | | | | QAVPLPEGLLRQRYREEKTLEERRWERLEFLQR | |
| | | | | | KKAFLRHVRRRHRDHMAPYAVGREARISPLGDR | |
| | | | | | SQNRFRCECRYCQSHRPNLSGIPGESNRAPHPS | |
| | | | | | SWETLVQGLSGLTLSLGTNQPGPLPEAALQPQET | |
| | | | | | EEKRQRERQQESKIMFQRLLKQWLEEN | |
| 412 | JHUO | NIV | 0.5659150 | FAM 15 | MMGLSNLSPGPGPSQAVPLPEGLLRQRYREEKT | Core Human ORF Only |
| | 2677 | OIPI | 5 | 6A | LEERRWERLEFLQRKKAFLRHVRRRHRDHMAPY | |
| | | _RF | | | AVGREARISPLGDRSQNRFRCECRYCQSHRPNL | |
| | | S | | | SGIPGESNRAPHPSSWETLVQGLSGLTLSLGTNQ | |
| | | | | | PGPLPEAALQPQETEEKRQRERQQESKIMFQRLL | |
| | | | | | KQWLEEN | |
| 413 | JHUO | NIV | 0.80700881 | MAGE | MSPILGYWKIKGLVQPTRLLLEYLEEKYEEHLYER | Full Literal Sequence- |
| | 2741 | OIPI | 9 | A3 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPLEQRSQHCKPEE | |
| | | | | | GLEARGEALGLVGAQAPATEEQEAASSSSTLVEV | |
| | | | | | TLGEVPAAESPDPPQSPQGASSLPTTMNYPLWS | |
| | | | | | QSYEDSSNQEEEGPSTFPDLESEFQAALSRKVAE | |
| | | | | | LVHFLLLKYRAREPVTKAEMLGSVVGNWQYFFPV | |
| | | | | | IFSKASSSLQLVFGIELMEVDPIGHLYIFATCLGLS | |
| | | | | | YDGLLGDNQIMPKAGLLIIVLAIIAREGDCAPEEKI | |
| | | | | | WEELSVLEVFEGREDSILGDPKKLLTQHFVQENY | |
| | | | | | LEYRQVPGSDPACYEFLWGPRALVETSYVKVLH | |
| | | | | | HMVKISGGPHISYPPLHEWVLREGEE | |
| 414 | JHUO | NIV | 0.80700881 | MAGE | MPLEQRSQHCKPEEGLEARGEALGLVGAQAPAT | Core Human ORF Only |
| | 2741 | OIPI | 9 | A3 | EEQEAASSSSTLVEVTLGEVPAAESPDPPQSPQG | |
| | | _RF | | | ASSLPTTMNYPLWSQSYEDSSNQEEEGPSTFPD | |
| | | S | | | LESEFQAALSRKVAELVHFLLLKYRAREPVTKAE | |
| | | | | | MLGSVVGNWQYFFPVIFSKASSSLQLVFGIELME | |
| | | | | | VDPIGHLYIFATCLGLSYDGLLGDNQIMPKAGLLII | |
| | | | | | VLAHAREGDCAPEEKIWEELSVLEVFEGREDSILG | |
| | | | | | DPKKLLTQHFVQENYLEYRQVPGSDPACYEFLW | |
| | | | | | GPRALVETSYVKVLHHMVKISGGPHISYPPLHEW | |
| | | | | | VLREGEE | |
| 415 | JHUO | NIV | 0.23082149 | MAGE | MSPILGYWKIKGLVQPTRLLLEYLEEKYEEHLYER | Full Literal Sequence- |
| | 2741 | OIPI | 6 | A3 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPLEQRSQHCKPEE | |
| | | | | | GLEARGEALGLVGAQAPATEEQEAASSSSTLVEV | |
| | | | | | TLGEVPAAESPDPPQSPQGASSLPTTMNYPLWS | |
| | | | | | QSYEDSSNQEEEGPSTFPDLESEFQAALSRKVAE | |
| | | | | | LVHFLLLKYRAREPVTKAEMLGSVVGNWQYFFPV | |
| | | | | | IFSKASSSLQLVFGIELMEVDPIGHLYIFATCLGLS | |
| | | | | | YDGLLGDNQIMPKAGLLIIVLAIIAREGDCAPEEKI | |
| | | | | | WEELSVLEVFEGREDSILGDPKKLLTQHFVQENY | |
| | | | | | LEYRQVPGSDPACYEFLWGPRALVETSYVKVLH | |
| | | | | | HMVKISGGPHISYPPLHEWVLREGEE | |
| 416 | JHUO | NIV | 0.23082149 | MAGE | MPLEQRSQHCKPEEGLEARGEALGLVGAQAPAT | Core Human ORF Only |
| | 2741 | OIPI | 6 | A3 | EEQEAASSSSTLVEVTLGEVPAAESPDPPQSPQG | |
| | | _RF | | | ASSLPTTMNYPLWSQSYEDSSNQEEEGPSTFPD | |
| | | S | | | LESEFQAALSRKVAELVHFLLLKYRAREPVTKAE | |
| | | | | | MLGSVVGNWQYFFPVIFSKASSSLQLVFGIELME | |
| | | | | | VDPIGHLYIFATCLGLSYDGLLGDNQIMPKAGLLII | |
| | | | | | VLAHAREGDCAPEEKIWEELSVLEVFEGREDSILG | |
| | | | | | DPKKLLTQHFVQENYLEYRQVPGSDPACYEFLW | |
| | | | | | GPRALVETSYVKVLHHMVKISGGPHISYPPLHEW | |
| | | | | | VLREGEE | |
| 417 | JHUO | NIV | 0.51134908 | CD177 | MSPILGYWKIKGLVQPTRLLLEYLEEKYEEHLYER | Full Literal Sequence- |
| | 2993 | OIPI | 3 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSPVLLLALLGFILPL | |
| | | | | | PGVQALLCQFGTVQHVWKVSDLPRQWTPKNTS | |
| | | | | | CDSGLGCQDTLMLIESGPQVSLVLSKGCTEAKDQ | |
| | | | | | EPRVTEHRMGPGLSLISYTFVCRQEDFCNNLVNS | |
| | | | | | LPLWAPQPPADPGSLRCPVCLSMEGCLEGTTEEI | |
| | | | | | CPKGTTHCYDGLLRLRGGGIFSNLRVQGCMPQP | |
| | | | | | GCNLLNGTQEIGPVGMTENCNRKDFLTCHRGTTI | |
| | | | | | MTHGNLAQEPTDWTTSNTEMCEVGQVCQETLLL | |
| | | | | | LDVGLTSTLVGTKGCSTVGAQNSQKTTIHSAPPG | |
| | | | | | VLVASYTHFCSSDLCNSASSSSVLLNSLPPQAAP | |
| | | | | | VPGDRQCPTCVQPLGTCSSGSPRMTCPRGTTH | |
| | | | | | CYDGYIHLSGGGLSTKMSIQGCVAQPSSFLLNHT | |
| | | | | | RQIGIFSAREKRDVQPPASQHEGGGAEGLESLT | |
| | | | | | WGVGLALAPALWWGVVCPSC | |
| 418 | JHUO | NIV | 0.51134908 | CD177 | MSPVLLLALLGFILPLPGVQALLCQFGTVQHVWK | Core Human ORF Only |
| | 2993 | OIPI | 3 | | VSDLPRQWTPKNTSCDSGLGCQDTLMLIESGPQ | |
| | | _RF | | | VSLVLSKGCTEAKDQEPRVTEHRMGPGLSLISYT | |
| | | S | | | FVCRQEDFCNNLVNSLPLWAPQPPADPGSLRCP | |
| | | | | | VCLSMEGCLEGTTEEICPKGTTHCYDGLLRLRGG | |
| | | | | | GIFSNLRVQGCMPQPGCNLLNGTQEIGPVGMTE | |
| | | | | | NCNRKDFLTCHRGTTIMTHGNLAQEPTDWTTSN | |
| | | | | | TEMCEVGQVCQETLLLLDVGLTSTLVGTKGCSTV | |
| | | | | | GAQNSQKTTIHSAPPGVLVASYTHFCSSDLCNSA | |
| | | | | | SSSSVLLNSLPPQAAPVPGDRQCPTCVQPLGTC | |
| | | | | | SSGSPRMTCPRGTTHCYDGYIHLSGGGLSTKMSI | |
| | | | | | QGCVAQPSSFLLNHTRQIGIFSAREKRDVQPPAS | |
| | | | | | QHEGGGAEGLESLTWGVGLALAPALVWVGVVCP | |
| | | | | | SC | |
| 419 | JHUO | NIV | 0.90764497 | NUP13 | MSPILGYWKIKGLVQPTRLLLEYLEEKYEEHLYER | Full Literal Sequence- |
| | 3609 | OIPI | 4 | 3 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMFPAAPSPRTPGTG | |
| | | | | | SRRGPLAGLGPGSTPRTASRKGLPLGSAVSSPVL | |
| | | | | | FSPVGRRSSLSSRGTPTRMFPHHSITESVNYDVK | |
| | | | | | TFGSSLPVKVMEALTLAEVDDQLTINIDEGGWACL | |
| | | | | | VCKEKLIIWKIALSPITKLSVCKELQLPPSDFHWSA | |
| | | | | | DLVALSYSSPSGEAHSTQAVAVMVATREGSIRYW | |
| | | | | | PSLAGEDTYTEAFVDSGGDKTYSFLTAVQGGSFI | |
| | | | | | LSSSGSQLIRLIPESSGKIHQHILPQGQGMLSGIG | |
| | | | | | RKVSSLFGILSPSSDLTLSSVLWDRERSSFYSLTS | |
| | | | | | SNISKWELDDSSEKHAYSWDINRALKENITDAIWG | |
| | | | | | SESNYEAIKEGVNIRYLDFKQNCDGLVILAAAWHS | |
| | | | | | ADNPCLIYYSLITIEDNGCQMSDAVTVEVTQYNPP | |
| | | | | | FQSEDLILCQLTVPNFSNQTAYLYNESAVYVCST | |
| | | | | | GTGKFSLPQEKIVFNAQGDSVLGAGACGGVPIIFS | |
| | | | | | RNSGLVSITSRENVSILAEDLEGSLASSVAGPNSE | |
| | | | | | SMIFETTTKNETIAQEDKIKLLKAAFLQYCRKDLGH | |
| | | | | | AQMVVDELFSSHSDLDSDSELDRAVTQISVDLMD | |
| | | | | | DYPASDPRWAESVPEEAPGFSNTSLIILHQLEDK | |
| | | | | | MKAHSFLMDFIHQVGLFGRLGSFPVRGTPMATRL | |
| | | | | | LLCEHAEKLSAAIVLKNHHSRLSDLVNTAILIALNK | |
| | | | | | REYEIPSNLTPADVFFREVSQVDTICECLLEHEEQ | |
| | | | | | VLRDAPMDSIEWAEVVINVNNILKDMLQAASHYR | |
| | | | | | QNRNSLYRREESLEKEPEYVPWTATSGPGGIRTV | |
| | | | | | IIRQHEIVLKVAYPQADSNLRNIVTEQLVALIDCFLD | |
| | | | | | GYVSQLKSVDKSSNRERYDNLEMEYLQKRSDLL | |
| | | | | | SPLLSLGQYLWAASLAEKYCDFDILVQMCEQTDN | |
| | | | | | QSRLQRYMTQFADQNFSDFLFRWYLEKGKRGKL | |
| | | | | | LSQPISQHGQLANFLQAHEHLSWLHEINSQELEK | |
| | | | | | AHATLLGLANMETRYFAKKKTLLGLSKLAALASDF | |
| | | | | | SEDMLQEKIEEMAEQERFLLHQETLPEQLLAEKQ | |
| | | | | | LNLSAMPVLTAPQLIGLYICEENRRANEYDFKKAL | |
| | | | | | DLLEYIDEEEDININDLKLEILCKALQRDNWSSSDG | |
| | | | | | KDDPIEVSKDSIFVKILQKLLKDGIQLSEYLPEVKD | |
| | | | | | LLQADQLGSLKSNPYFEFVLKANYEYYVQGQI | |
| 420 | JHUO | NIV | 0.90764497 | NUP13 | MFPAAPSPRTPGTGSRRGPLAGLGPGSTPRTAS | Core Human ORF Only |
| | 3609 | OIPI | 4 | 3 | RKGLPLGSAVSSPVLFSPVGRRSSLSSRGTPTRM | |
| | | _RF | | | FPHHSITESVNYDVKTFGSSLPVKVMEALTLAEVD | |
| | | S | | | DQLTINIDEGGWACLVCKEKLIIWKIALSPITKLSVC | |
| | | | | | KELQLPPSDFHWSADLVALSYSSPSGEAHSTQAV | |
| | | | | | AVMVATREGSIRYWPSLAGEDTYTEAFVDSGGD | |
| | | | | | KTYSFLTAVQGGSFILSSSGSQLIRLIPESSGKIHQ | |
| | | | | | HILPQGQGMLSGIGRKVSSLFGILSPSSDLTLSSV | |
| | | | | | LWDRERSSFYSLTSSNISKWELDDSSEKHAYSW | |
| | | | | | DINRALKENITDAIWGSESNYEAIKEGVNIRYLDFK | |
| | | | | | QNCDGLVILAAAWHSADNPCLIYYSLITIEDNGCQ | |
| | | | | | MSDAVTVEVTQYNPPFQSEDLILCQLTVPNFSNQ | |
| | | | | | TAYLYNESAVYVCSTGTGKFSLPQEKIVFNAQGD | |
| | | | | | SVLGAGACGGVPIIFSRNSGLVSITSRENVSILAED | |
| | | | | | LEGSLASSVAGPNSESMIFETTTKNETIAQEDKIKL | |
| | | | | | LKAAFLQYCRKDLGHAQMVVDELFSSHSDLDSD | |
| | | | | | SELDRAVTQISVDLMDDYPASDPRWAESVPEEAP | |
| | | | | | GFSNTSLIILHQLEDKMKAHSFLMDFIHQVGLFGR | |
| | | | | | LGSFPVRGTPMATRLLLCEHAEKLSAAIVLKNHHS | |
| | | | | | RLSDLVNTAILIALNKREYEIPSNLTPADVFFREVS | |
| | | | | | QVDTICECLLEHEEQVLRDAPMDSIEWAEVVINVN | |
| | | | | | NILKDMLQAASHYRQNRNSLYRREESLEKEPEYV | |
| | | | | | PWTATSGPGGIRTVIIRQHEIVLKVAYPQADSNLR | |
| | | | | | NIVTEQLVALIDCFLDGYVSQLKSVDKSSNRERYD | |
| | | | | | NLEMEYLQKRSDLLSPLLSLGQYLWAASLAEKYC | |
| | | | | | DFDILVQMCEQTDNQSRLQRYMTQFADQNFSDF | |
| | | | | | LFRWYLEKGKRGKLLSQPISQHGQLANFLQAHEH | |
| | | | | | LSWLHEINSQELEKAHATLLGLANMETRYFAKKK | |
| | | | | | TLLGLSKLAALASDFSEDMLQEKIEEMAEQERFLL | |
| | | | | | HQETLPEQLLAEKQLNLSAMPVLTAPQLIGLYICE | |
| | | | | | ENRRANEYDFKKALDLLEYIDEEEDININDLKLEIL | |
| | | | | | CKALQRDNWSSSDGKDDPIEVSKDSIFVKILQKLL | |
| | | | | | KDGIQLSEYLPEVKDLLQADQLGSLKSNPYFEFVL | |
| | | | | | KANYEYYVQGQI | |
| 421 | JHUO | NIV | 0.95708839 | ZNF62 | MSPILGYWKIKGLVQPTRLLLEYLEEKYEEHLYER | Full Literal Sequence- |
| | 3647 | OIPI | 7 | 2 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMATYTCITCRVAFRD | |
| | | | | | ADMQRAHYKTDWHRYNLRRKVASMAPVTAEGF | |
| | | | | | QERVRAQRAVAEEESKGSATYCTVCSKKFASFN | |
| | | | | | AYENHLKSRRHVELEKKAVQAVNRKVEMMNEKN | |
| | | | | | LEKGLGVDSVDKDAMNAAIQQAIKAQPSMSPKKA | |
| | | | | | PPAPAKEARNVVAVGTGGRGTHDRDPSEKPPRL | |
| | | | | | QWFEQQAKKLAKQQEEDSEEEEEDLDGDDWEDI | |
| | | | | | DSDEELECEDTEAMDDVVEQDAEEEEAEEGPPL | |
| | | | | | GAIPITDCLFCSHHSSSLMKNVAHMTKDHSFFIPDI | |
| | | | | | EYLSDIKGLIKYLGEKVGVGKICLWCNEKGKSFYS | |
| | | | | | TEAVQAHMNDKSHCKLFTDGDAALEFADFYDFR | |
| | | | | | SSYPDHKEGEDPNKAEELPSEKNLEYDDETMELI | |
| | | | | | LPSGARVGHRSLMRYYKQRFGLSRAVAVAKNRK | |
| | | | | | AVGRVLQQYRALGWTGSTGAALMRERDMQYVQ | |
| | | | | | RMKSKWMLKTGMKNNATKQMHFRVQVRF | |
| 422 | JHUO | NIV | 0.95708839 | ZNF62 | MATYTCITCRVAFRDADMQRAHYKTDWHRYNLR | Core Human ORF Only |
| | 3647 | OIPI | 7 | 2 | RKVASMAPVTAEGFQERVRAQRAVAEEESKGSA | |
| | | _RF | | | TYCTVCSKKFASFNAYENHLKSRRHVELEKKAVQ | |
| | | S | | | AVNRKVEMMNEKNLEKGLGVDSVDKDAMNAAIQ | |
| | | | | | QAIKAQPSMSPKKAPPAPAKEARNVVAVGTGGR | |
| | | | | | GTHDRDPSEKPPRLQWFEQQAKKLAKQQEEDSE | |
| | | | | | EEEEDLDGDDWEDIDSDEELECEDTEAMDDVVE | |
| | | | | | QDAEEEEAEEGPPLGAIPITDCLFCSHHSSSLMK | |
| | | | | | NVAHMTKDHSFFIPDIEYLSDIKGLIKYLGEKVGVG | |
| | | | | | KICLWCNEKGKSFYSTEAVQAHMNDKSHCKLFT | |
| | | | | | DGDAALEFADFYDFRSSYPDHKEGEDPNKAEEL | |
| | | | | | PSEKNLEYDDETMELILPSGARVGHRSLMRYYKQ | |
| | | | | | RFGLSRAVAVAKNRKAVGRVLQQYRALGWTGST | |
| | | | | | GAALMRERDMQYVQRMKSKWMLKTGMKNNATK | |
| | | | | | QMHFRVQVRF | |
| 423 | JHUO | NIV | 1.33772506 | UBA6 | MSPILGYWKIKGLVQPTRLLLEYLEEKYEEHLYER | Full Literal Sequence- |
| | 4218 | OIPI | 6 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEGSEPVAAHQGEE | |
| | | | | | ASCSSWGTGSTNKNLPIMSTASVEIDDALYSRQR | |
| | | | | | YVLGDTAMQKMAKSHVFLSGMGGLGLEIAKNLVL | |
| | | | | | AGIKAVTIHDTEKCQAWDLGTNFFLSEDDVVNKR | |
| | | | | | NRAEAVLKHIAELNPYVHVTSSSVPFNETTDLSFL | |
| | | | | | DKYQCVVLTEMKLPLQKKINDFCRSQCPPIKFISA | |
| | | | | | DVHGIWSRLFCDFGDEFEVLDTTGEEPKEIFISNIT | |
| | | | | | QANPGIVTCLENHPHKLETGQFLTFREINGMTGL | |
| | | | | | NGSIQQITVISPFSFSIGDTTELEPYLHGGIAVQVK | |
| | | | | | TPKTVFFESLERQLKHPKCLIVDFSNPEAPLEIHTA | |
| | | | | | MLALDQFQEKYSRKPNVGCQQDSEELLKLATSIS | |
| | | | | | ETLEEKVTIEIYGCPNICLLIHKCSVY | |
| 424 | JHUO | NIV | 1.33772506 | UBA6 | MEGSEPVAAHQGEEASCSSWGTGSTNKNLPIMS | Core Human ORF Only |
| | 4218 | OIPI | 6 | | TASVEIDDALYSRQRYVLGDTAMQKMAKSHVFLS | |
| | | _RF | | | GMGGLGLEIAKNLVLAGIKAVTIHDTEKCQAWDL | |
| | | S | | | GTNFFLSEDDVVNKRNRAEAVLKHIAELNPYVHV | |
| | | | | | TSSSVPFNETTDLSFLDKYQCVVLTEMKLPLQKKI | |
| | | | | | NDFCRSQCPPIKFISADVHGIWSRLFCDFGDEFE | |
| | | | | | VLDTTGEEPKEIFISNITQANPGIVTCLENHPHKLE | |
| | | | | | TGQFLTFREINGMTGLNGSIQQITVISPFSFSIGDT | |
| | | | | | TELEPYLHGGIAVQVKTPKTVFFESLERQLKHPKC | |
| | | | | | LIVDFSNPEAPLEIHTAMLALDQFQEKYSRKPNVG | |
| | | | | | CQQDSEELLKLATSISETLEEKVTIEIYGCPNICLLI | |
| | | | | | HKCSVY | |
| 425 | JHUO | NIV | 0.47659465 | ERCC8 | MSPILGYWKIKGLVQPTRLLLEYLEEKYEEHLYER | Full Literal Sequence- |
| | 4440 | OIPI | 4 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLGFLSARQTGLED | |
| | | | | | PLRLRRAESTRRVLGLELNKDRDVERIHGGGINTL | |
| | | | | | DIEPVEGRYMLSGGSDGVIVLYDLENSSRQSYYT | |
| | | | | | CKAVCSIGRDHPDVHRYSVETVQWYPHDTGMFT | |
| | | | | | SSSFDKTLKVWDTNTLQTADVFNFEETVYSHHMS | |
| | | | | | PVSTKHCLVAVGTRGPKVQLCDLKSGSCSHILQG | |
| | | | | | IFILFQTATTLSKRFNKKKRY | |
| 426 | JHUO | NIV | 0.47659465 | ERCC8 | MLGFLSARQTGLEDPLRLRRAESTRRVLGLELNK | Core Human ORF Only |
| | 4440 | OIPI | 4 | | DRDVERIHGGGINTLDIEPVEGRYMLSGGSDGVIV | |
| | | _RF | | | LYDLENSSRQSYYTCKAVCSIGRDHPDVHRYSVE | |
| | | S | | | TVQWYPHDTGMFTSSSFDKTLKVWDTNTLQTAD | |
| | | | | | VFNFEETVYSHHMSPVSTKHCLVAVGTRGPKVQ | |
| | | | | | LCDLKSGSCSHILQGIFILFQTATTLSKRFNKKKRY | |
| 427 | JHUO | NIV | 0.90722848 | CBX3 | MSPILGYWKIKGLVQPTRLLLEYLEEKYEEHLYER | Full Literal Sequence- |
| | 6273 | OIPI | 1 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASNKTTLQKMGKK | |
| | | | | | QNGKSKKVEEAEPEEFVVEKVLDRRVVNGKVEY | |
| | | | | | FLKWKGFTDADNTWEPEENLDCPELIEAFLNSQK | |
| | | | | | AGKEKDGTKRKSLSDSESDDSKSKKKRDAADKP | |
| | | | | | RGFARGLDPERIIGATDSSGELMFLMKWKDSDEA | |
| | | | | | DLVLAKEANMKCPQIVIAFYEERLTWHSCPEDEA | |
| | | | | | Q | |
| 428 | JHUO | NIV | 0.90722848 | CBX3 | MASNKTTLQKMGKKQNGKSKKVEEAEPEEFVVE | Core Human ORF Only |
| | 6273 | OIPI | 1 | | KVLDRRVVNGKVEYFLKWKGFTDADNTWEPEEN | |
| | | _RF | | | LDCPELIEAFLNSQKAGKEKDGTKRKSLSDSESD | |
| | | S | | | DSKSKKKRDAADKPRGFARGLDPERIIGATDSSG | |
| | | | | | ELMFLMKWKDSDEADLVLAKEANMKCPQIVIAFY | |
| | | | | | EERLTWHSCPEDEAQ | |
| 429 | JHUO | NIV | 0.38294515 | SIK1 | MSPILGYWKIKGLVQPTRLLLEYLEEKYEEHLYER | Full Literal Sequence- |
| | 6326 | OIPI | 8 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVIMSEFSADPAGQ | |
| | | | | | GQGQQKPLRVGFYDIERTLGKGNFAVVKLARHR | |
| | | | | | VTKTQVAIKIIDKTRLDSSNLEKIYREVQLMKLLNH | |
| | | | | | PHIIKLYQVMETKDMLYIVTEFAKNGEMFDYLTSN | |
| | | | | | GHLSENEARKKFWQILSAVEYCHDHHIVHRDLKT | |
| | | | | | ENLLLDGNMDIKLADFGFGNFYKSGEPLSTWCGS | |
| | | | | | PPYAAPEVFEGKEYEGPQLDIWSLGVVLYVLVCG | |
| | | | | | SLPFDGPNLPTLRQRVLEGRFRIPFFMSQDCESLI | |
| | | | | | RRMLVVDPARRITIAQIRQHRWMRAEPCLPGPAC | |
| | | | | | PAFSAHSYTSNLGDYDEQALGIMQTLGVDRQRTV | |
| | | | | | ESLQNSSYNHFAAIYYLLLERLKEYRNAQCARPG | |
| | | | | | PARQPRPRSSDLSGLEVPQEGLSTDPFRPALLCP | |
| | | | | | QPQTLVQSVLQAEMDCELQSSLQWPLFFPVDAS | |
| | | | | | CSGVFRPRPVSPSSLLDTAISEEARQGPGLEEEQ | |
| | | | | | DTQESLPSSTGRRHTLAEVSTRLSPLTAPCIVVSP | |
| | | | | | STTASPAEGTSSDSCLTFSASKSPAGLSGTPATQ | |
| | | | | | GLLGACSPVRLASPFLGSQSATPVLQAQGGLGG | |
| | | | | | AVLLPVSFQEGRRASDTSLTQGLKAFRQQLRKTT | |
| | | | | | RTKGFLGLNKIKGLARQVCQVPASRASRGGLSPF | |
| | | | | | HAPAQSPGLHGGAAGSREGWSLLEEVLEQQRLL | |
| | | | | | QLQHHPAAAPGCSQAPQPAPAPFVIAPCDGPGA | |
| | | | | | APLPSTLLTSGLPLLPPPLLQTGASPVASAAQLLD | |
| | | | | | THLHIGTGPTALPAVPPPRLARLAPGCEPLGLLQG | |
| | | | | | DCEMEDLMPCSLGTFVLVQ | |
| 430 | JHUO | NIV | 0.38294515 | SIK1 | MVIMSEFSADPAGQGQGQQKPLRVGFYDIERTL | Core Human ORF Only |
| | 6326 | OIPI | 8 | | GKGNFAVVKLARHRVTKTQVAIKIIDKTRLDSSNL | |
| | | _RF | | | EKIYREVQLMKLLNHPHIIKLYQVMETKDMLYIVTE | |
| | | S | | | FAKNGEMFDYLTSNGHLSENEARKKFWQILSAVE | |
| | | | | | YCHDHHIVHRDLKTENLLLDGNMDIKLADFGFGN | |
| | | | | | FYKSGEPLSTWCGSPPYAAPEVFEGKEYEGPQL | |
| | | | | | DIWSLGVVLYVLVCGSLPFDGPNLPTLRQRVLEG | |
| | | | | | RFRIPFFMSQDCESLIRRMLVVDPARRITIAQIRQH | |
| | | | | | RWMRAEPCLPGPACPAFSAHSYTSNLGDYDEQA | |
| | | | | | LGIMQTLGVDRQRTVESLQNSSYNHFAAIYYLLLE | |
| | | | | | RLKEYRNAQCARPGPARQPRPRSSDLSGLEVPQ | |
| | | | | | EGLSTDPFRPALLCPQPQTLVQSVLQAEMDCELQ | |
| | | | | | SSLQWPLFFPVDASCSGVFRPRPVSPSSLLDTAI | |
| | | | | | SEEARQGPGLEEEQDTQESLPSSTGRRHTLAEV | |
| | | | | | STRLSPLTAPCIVVSPSTTASPAEGTSSDSCLTFS | |
| | | | | | ASKSPAGLSGTPATQGLLGACSPVRLASPFLGSQ | |
| | | | | | SATPVLQAQGGLGGAVLLPVSFQEGRRASDTSLT | |
| | | | | | QGLKAFRQQLRKTTRTKGFLGLNKIKGLARQVCQ | |
| | | | | | VPASRASRGGLSPFHAPAQSPGLHGGAAGSREG | |
| | | | | | WSLLEEVLEQQRLLQLQHHPAAAPGCSQAPQPA | |
| | | | | | PAPFVIAPCDGPGAAPLPSTLLTSGLPLLPPPLLQ | |
| | | | | | TGASPVASAAQLLDTHLHIGTGPTALPAVPPPRLA | |
| | | | | | RLAPGCEPLGLLQGDCEMEDLMPCSLGTFVLVQ | |
| 431 | JHUO | NIV | 0.3386631 | LILRB5 | MSPILGYWKIKGLVQPTRLLLEYLEEKYEEHLYER | Full Literal Sequence- |
| | 6378 | OIPI | 7 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTLTLSVLICLGLSVG | |
| | | | | | PRTCVQAGTLPKPTLWAEPASVIARGKPVTLWCQ | |
| | | | | | GPLETEEYRLDKEGLPWARKRQNPLEPGAKAKF | |
| | | | | | HIPSTVYDSAGRYRCYYETPAGWSEPSDPLELVA | |
| | | | | | TGFYAEPTLLALPSPVVASGGNVTLQCDTLDGLL | |
| | | | | | TFVLVEEEQKLPRTLYSQKLPKGPSQALFPVGPV | |
| | | | | | TPSCRWRFRCYYYYRKNPQVWSNPSDLLEILVP | |
| | | | | | GVSRKPSLLIPQGSVVARGGSLTLQCRSDVGYDI | |
| | | | | | FVLYKEGEHDLVQGSGQQPQAGLSQANFTLGPV | |
| | | | | | SRSHGGQYRCYGAHNLSPRWSAPSDPLDILIAGL | |
| | | | | | IPDIPALSVQPGPKVASGENVTLLCQSWHQIDTFF | |
| | | | | | LTKEGAAHPPLCLKSKYQSYRHQAEFSMSPVTSA | |
| | | | | | QGGTYRCYSAIRSYPYLLSSPSYPQELVVSGPSG | |
| | | | | | DPSLSPTGSTPTPGPEDQPLTPTGLDPQSGLGR | |
| | | | | | HLGVVTGVSVAFVLLLFLLLFLLLRHRHQSKHRTS | |
| | | | | | AHFYRPAGAAGPEPKDQGLQKRASPVADIQEEIL | |
| | | | | | NAAVKDTQPKDGVEMDARAAASEAPQDVTYAQL | |
| | | | | | HSLTLRREATEPPPSQEREPPAEPSIYAPLAIH | |
| 432 | JHUO | NIV | 0.3386631 | LILRB5 | MTLTLSVLICLGLSVGPRTCVQAGTLPKPTLWAEP | Core Human ORF Only |
| | 6378 | OIPI | 7 | | ASVIARGKPVTLWCQGPLETEEYRLDKEGLPWA | |
| | | _RF | | | RKRQNPLEPGAKAKFHIPSTVYDSAGRYRCYYET | |
| | | S | | | PAGWSEPSDPLELVATGFYAEPTLLALPSPVVAS | |
| | | | | | GGNVTLQCDTLDGLLTFVLVEEEQKLPRTLYSQK | |
| | | | | | LPKGPSQALFPVGPVTPSCRWRFRCYYYYRKNP | |
| | | | | | QVWSNPSDLLEILVPGVSRKPSLLIPQGSVVARG | |
| | | | | | GSLTLQCRSDVGYDIFVLYKEGEHDLVQGSGQQ | |
| | | | | | PQAGLSQANFTLGPVSRSHGGQYRCYGAHNLSP | |
| | | | | | RWSAPSDPLDILIAGLIPDIPALSVQPGPKVASGE | |
| | | | | | NVTLLCQSWHQIDTFFLTKEGAAHPPLCLKSKYQ | |
| | | | | | SYRHQAEFSMSPVTSAQGGTYRCYSAIRSYPYLL | |
| | | | | | SSPSYPQELVVSGPSGDPSLSPTGSTPTPGPED | |
| | | | | | QPLTPTGLDPQSGLGRHLGVVTGVSVAFVLLLFL | |
| | | | | | LLFLLLRHRHQSKHRTSAHFYRPAGAAGPEPKDQ | |
| | | | | | GLQKRASPVADIQEEILNAAVKDTQPKDGVEMDA | |
| | | | | | RAAASEAPQDVTYAQLHSLTLRREATEPPPSQER | |
| | | | | | EPPAEPSIYAPLAIH | |
| 433 | JHUO | NIV | 0.88504287 | AGMAT | MSPILGYWKIKGLVQPTRLLLEYLEEKYEEHLYER | Full Literal Sequence- |
| | 8736 | OIPI | 8 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLRLLASGCARGPG | |
| | | | | | PGVGARPAAGLFHPGRRQSRQASDAPRNQPPS | |
| | | | | | PEFVARPVGVCSMMRLPVQTSPEGLDAAFIGVPL | |
| | | | | | DTGTSNRPGARFGPRRIREESVMLRTVNPSTGAL | |
| | | | | | PFQSLMVADLGDVNVNLYNLQDSCRRIQEAYEKI | |
| | | | | | VAAGCIPLTLGGDHTITYPILQAMAKKHGPVGLLH | |
| | | | | | VDAHTDTTDKALGEKLYHGAPFRRCVDEGLLDCK | |
| | | | | | RVVQIGIRGSSTTLDPYRYNRSQGFRVVLAEDCW | |
| | | | | | MKSLVPLMGEVRQQMGGKPIYISFDIDALDPAYA | |
| | | | | | PGTGTPEIAGLTPSQALEIIRGCQGLNVMGCDLVE | |
| | | | | | VSPPYDLSGNTALLAANLLFEMLCALPKVTTV | |
| 434 | JHUO | NIV | 0.88504287 | AGMAT | MLRLLASGCARGPGPGVGARPAAGLFHPGRRQS | Core Human ORF Only |
| | 8736 | OIPI | 8 | | RQASDAPRNQPPSPEFVARPVGVCSMMRLPVQT | |
| | | _RF | | | SPEGLDAAFIGVPLDTGTSNRPGARFGPRRIREE | |
| | | S | | | SVMLRTVNPSTGALPFQSLMVADLGDVNVNLYNL | |
| | | | | | QDSCRRIQEAYEKIVAAGCIPLTLGGDHTITYPILQ | |
| | | | | | AMAKKHGPVGLLHVDAHTDTTDKALGEKLYHGA | |
| | | | | | PFRRCVDEGLLDCKRVVQIGIRGSSTTLDPYRYN | |
| | | | | | RSQGFRVVLAEDCWMKSLVPLMGEVRQQMGGK | |
| | | | | | PIYISFDIDALDPAYAPGTGTPEIAGLTPSQALEIIR | |
| | | | | | GCQGLNVMGCDLVEVSPPYDLSGNTALLAANLLF | |
| | | | | | EMLCALPKVTTV | |
| 435 | JHUO | NIV | 0.09401257 | FJX1 | MSPILGYWKIKGLVQPTRLLLEYLEEKYEEHLYER | Full Literal Sequence- |
| | 9050 | OIPI | 6 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGRRMRGAAATAGL | |
| | | | | | WLLALGSLLALWGGLLPPRTELPASRPPEDRLPR | |
| | | | | | RPARSGGPAPAPRFPLPPPLAWDARGGSLKTFR | |
| | | | | | ALLTLAAGADGPPRQSRSEPRWHVSARQPRPEE | |
| | | | | | SAAVHGGVFWSRGLEEQVPPGFSEAQAAAWLE | |
| | | | | | AARGARMVALERGGCGRSSNRLARFADGTRAC | |
| | | | | | VRYGINPEQIQGEALSYYLARLLGLQRHVPPLALA | |
| | | | | | RVEARGAQWAQVQEELRAAHWTEGSVVSLTRW | |
| | | | | | LPNLTDVVVPAPWRSEDGRLRPLRDAGGELANL | |
| | | | | | SQAELVDLVQWTDLILFDYLTANFDRLVSNLFSLQ | |
| | | | | | WDPRVMQRATSNLHRGPGGALVFLDNEAGLVH | |
| | | | | | GYRVAGMWDKYNEPLLQSVCVFRERTARRVLEL | |
| | | | | | HRGQDAAARLLRLYRRHEPRFPELAALADPHAQL | |
| | | | | | LQRRLDFLAKHILHCKAKYGRRSGT | |
| 436 | JHUO | NIV | 0.094012576 | FJX1 | MGRRMRGAAATAGLWLLALGSLLALWGGLLPPR | Core Human ORF Only |
| | 9050 | OIPI | | | TELPASRPPEDRLPRRPARSGGPAPAPRFPLPPP | |
| | | _RF | | | LAWDARGGSLKTFRALLTLAAGADGPPRQSRSE | |
| | | S | | | PRWHVSARQPRPEESAAVHGGVFWSRGLEEQV | |
| | | | | | PPGFSEAQAAAWLEAARGARMVALERGGCGRS | |
| | | | | | SNRLARFADGTRACVRYGINPEQIQGEALSYYLA | |
| | | | | | RLLGLQRHVPPLALARVEARGAQWAQVQEELRA | |
| | | | | | AHWTEGSVVSLTRWLPNLTDVVVPAPWRSEDGR | |
| | | | | | LRPLRDAGGELANLSQAELVDLVQWTDLILFDYLT | |
| | | | | | ANFDRLVSNLFSLQWDPRVMQRATSNLHRGPGG | |
| | | | | | ALVFLDNEAGLVHGYRVAGMWDKYNEPLLQSVC | |
| | | | | | VFRERTARRVLELHRGQDAAARLLRLYRRHEPRF | |
| | | | | | PELAALADPHAQLLQRRLDFLAKHILHCKAKYGR | |
| | | | | | RSGT | |
| 437 | JHUO | NIV | 1.19327236 | CTDSP | MSPILGYWKIKGLVQPTRLLLEYLEEKYEEHLYER | Full Literal Sequence- |
| | 9335 | OIPI | 3 | L2 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMRLRTRKASQQSNQ | |
| | | | | | IQTQRTARAKRKYSEVDDSLPSGGEKPSKNETGL | |
| | | | | | LSSIKKFIKGSTPKEERENPSKRSRIERDIDNNLITS | |
| | | | | | TPRAGEKPNKQISRVRRKSQVNGEAGSYEMTNQ | |
| | | | | | HVKQNGKLEDNPSSGSPPRTTLLGTIFSPVFNFF | |
| | | | | | SPANKNGTSGSDSPGQAVEAEEIVKQLDMEQVD | |
| | | | | | EITTSTTTSTNGAAYSNQAVQVRPSLNNGLEEAE | |
| | | | | | ETVNRDIPPLTAPVTPDSGYSSAHAEATYEEDWE | |
| | | | | | VFDPYYFIKHVPPLTEEQLNRKPALPLKTRSTPEF | |
| | | | | | SLVLDLDETLVHCSLNELEDAALTFPVLFQDVIYQ | |
| | | | | | VYVRLRPFFREFLERMSQMYEIILFTASKKVYADK | |
| | | | | | LLNILDPKKQLVRHRLFREHCVCVQGNYIKDLNIL | |
| | | | | | GRDLSKTIIIDNSPQAFAYQLSNGIPIESWFMDKN | |
| | | | | | DNELLKLIPFLEKLVELNEDVRPHIRDRFRLHDLLP | |
| | | | | | PD | |
| 438 | JHUO | NIV | 1.19327236 | CTDSP | MRLRTRKASQQSNQIQTQRTARAKRKYSEVDDS | Core Human ORF Only |
| | 9335 | OIPI | 3 | L2 | LPSGGEKPSKNETGLLSSIKKFIKGSTPKEERENP | |
| | | _RF | | | SKRSRIERDIDNNLITSTPRAGEKPNKQISRVRRK | |
| | | S | | | SQVNGEAGSYEMTNQHVKQNGKLEDNPSSGSP | |
| | | | | | PRTTLLGTIFSPVFNFFSPANKNGTSGSDSPGQA | |
| | | | | | VEAEEIVKQLDMEQVDEITTSTTTSTNGAAYSNQA | |
| | | | | | VQVRPSLNNGLEEAEETVNRDIPPLTAPVTPDSG | |
| | | | | | YSSAHAEATYEEDWEVFDPYYFIKHVPPLTEEQL | |
| | | | | | NRKPALPLKTRSTPEFSLVLDLDETLVHCSLNELE | |
| | | | | | DAALTFPVLFQDVIYQVYVRLRPFFREFLERMSQ | |
| | | | | | MYEHLFTASKKVYADKLLNILDPKKQLVRHRLFRE | |
| | | | | | HCVCVQGNYIKDLNILGRDLSKTIIIDNSPQAFAYQ | |
| | | | | | LSNGIPIESWFMDKNDNELLKLIPFLEKLVELNEDV | |
| | | | | | RPHIRDRFRLHDLLPPD | |
| 439 | JHUO | NIV | 2.08079814 | NCBP2 | MSPILGYWKIKGLVQPTRLLLEYLEEKYEEHLYER | Full Literal Sequence- |
| | 9933 | OIPI | 2 | -AS2 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVLRRLLAALLHSPQ | |
| | | | | | LVERLSESRPIRRAAQLTAFALLQAQLRGQDAAR | |
| | | | | | RLQDLAAGPVGSLCRRAERFRDAFTQELRRGLR | |
| | | | | | GRSGPPPGSQRGPGANI | |
| 440 | JHUO | NIV | 2.08079814 | NCBP2 | MVLRRLLAALLHSPQLVERLSESRPIRRAAQLTAF | Core Human ORF Only |
| | 9933 | OIPI | 2 | -AS2 | ALLQAQLRGQDAARRLQDLAAGPVGSLCRRAER | |
| | | _RF | | | FRDAFTQELRRGLRGRSGPPPGSQRGPGANI | |
| | | S | | | | |
| 441 | JHU1 | NIV | 1.4034522 | HTR4 | MSPILGYWKIKGLVQPTRLLLEYLEEKYEEHLYER | Full Literal Sequence- |
| | 0217 | OIPI | 4 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDKLDANVSSEEGF | |
| | | | | | GSVEKVVLLTFLSTVILMAILGNLLVMVAVCWDRQ | |
| | | | | | LRKIKTNYFIVSLAFADLLVSVLVMPFGAIELVQDI | |
| | | | | | WIYGEVFCLVRTSLDVLLTTASIFHLCCISLDRYYA | |
| | | | | | ICCQPLVYRNKMTPLRIALMLGGCVWIPTFI SFLPI | |
| | | | | | MQGWNNIGIIDLIEKRKFNQNSNSTYCVFMVNKP | |
| | | | | | YAITCSVVAFYIPFLLMVLAYYRIYVTAKEHAHQIQ | |
| | | | | | MLQRAGASSESRPQSADQHSTHRMRTETKAAKT | |
| | | | | | LCIIMGCFCLCWAPFFVTNIVDPFIDYTVPGQVWT | |
| | | | | | AFLWLGYINSGLNPFLYAFLNKSFRRAFLIILCCDD | |
| | | | | | ERYRRPSILGQTVPCSTTTINGSTHVLRDAVECG | |
| | | | | | GQWESQCHPPATSPLVAAQPSDT | |
| 442 | JHU1 | NIV | 1.4034522 | HTR4 | MDKLDANVSSEEGFGSVEKVVLLTFLSTVILMAIL | Core Human ORF Only |
| | 0217 | OIPI | 4 | | GNLLVMVAVCWDRQLRKIKTNYFIVSLAFADLLVS | |
| | | _RF | | | VLVMPFGAIELVQDIWIYGEVFCLVRTSLDVLLTTA | |
| | | S | | | SIFHLCCISLDRYYAICCQPLVYRNKMTPLRIALML | |
| | | | | | GGCWVIPTFISFLPIMQGWNNIGIIDLIEKRKFNQN | |
| | | | | | SNSTYCVFMVNKPYAITCSVVAFYIPFLLMVLAYY | |
| | | | | | RIYVTAKEHAHQIQMLQRAGASSESRPQSADQHS | |
| | | | | | THRMRTETKAAKTLCIIMGCFCLCWAPFFVTNIVD | |
| | | | | | PFIDYTVPGQVWTAFLWLGYINSGLNPFLYAFLNK | |
| | | | | | SFRRAFLIILCCDDERYRRPSILGQTVPCSTTTING | |
| | | | | | STHVLRDAVECGGQWESQCHPPATSPLVAAQPS | |
| | | | | | DT | |
| 443 | JHU1 | NIV | 0.55901184 | LOC10 | MSPILGYWKIKGLVQPTRLLLEYLEEKYEEHLYER | Full Literal Sequence- |
| | 1145 | OIPI | 5 | 272439 | DEGDKWRNKKFELGLEFPNLPYYIDGDVKLTQSM | (including GST-tag, 6- |
| | | _RF | | 8 | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMCLSSGHIRNEPRP | |
| | | | | | QSWPPPQGWVLRRPPQPLSLDLPAEQTRTSHSP | |
| | | | | | ATARSCSWSSTPGWRERRHPGQPASTQALTVE | |
| | | | | | AHVFLGSILKAVLSTTALTRIDLSEPACQYRPAQD | |
| | | | | | WPQGHIGEGGDQALAAAHVGKSMARQEVGPVS | |
| | | | | | LATATVGAGPGAPQP | |
| 444 | JHU1 | NIV | 0.55901184 | LOC10 | MCLSSGHIRNEPRPQSWPPPQGWVLRRPPQPLS | Core Human ORF Only |
| | 1145 | OIPI | 5 | 272439 | LDLPAEQTRTSHSPATARSCSWSSTPGWRERRH | |
| | | _RF | | 8 | PGQPASTQALTVEAHVFLGSILKAVLSTTALTRIDL | |
| | | S | | | SEPACQYRPAQDWPQGHIGEGGDQALAAAHVG | |
| | | | | | KSMARQEVGPVSLATATVGAGPGAPQP | |
| 445 | JHU1 | NIV | 0.51951288 | ISL1 | MSPILGYWKIKGLVQPTRLLLEYLEEKYEEHLYER | Full Literal Sequence- |
| | 2394 | OIPI | 6 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGDMGDPPKKKRLI | |
| | | | | | SLCVGCGNQIHDQYILRVSPDLEWHAACLKCAEC | |
| | | | | | NQYLDESCTCFVRDGKTYCKRDYIRLYGIKCAKC | |
| | | | | | SIGFSKNDFVMRARSKVYHIECFRCVACSRQLIPG | |
| | | | | | DEFALREDGLFCRADHDVVERASLGAGDPLSPLH | |
| | | | | | PARPLQMAAEPISARQPALRPHVHKQPEKTTRVR | |
| | | | | | TVLNEKQLHTLRTCYAANPRPDALMKEQLVEMTG | |
| | | | | | LSPRVIRVWFQNKRCKDKKRSIMMKQLQQQQPN | |
| | | | | | DKTNIQGMTGTPMVAASPERHDGGLQANPVEVQ | |
| | | | | | SYQPPWKVLSDFALQSDIDQPAFQQLVNFSEGG | |
| | | | | | PGSNSTGSEVASMSSQLPDTPNSMVASPIEA | |
| 446 | JHU1 | NIV | 0.51951288 | ISL1 | MGDMGDPPKKKRLISLCVGCGNQIHDQYILRVSP | Core Human ORF Only |
| | 2394 | OIPI | 6 | | DLEWHAACLKCAECNQYLDESCTCFVRDGKTYC | |
| | | _RF | | | KRDYIRLYGIKCAKCSIGFSKNDFVMRARSKVYHI | |
| | | S | | | ECFRCVACSRQLIPGDEFALREDGLFCRADHDVV | |
| | | | | | ERASLGAGDPLSPLHPARPLQMAAEPISARQPAL | |
| | | | | | RPHVHKQPEKTTRVRTVLNEKQLHTLRTCYAANP | |
| | | | | | RPDALMKEQLVEMTGLSPRVIRVWFQNKRCKDK | |
| | | | | | KRSIMMKQLQQQQPNDKTNIQGMTGTPMVAASP | |
| | | | | | ERHDGGLQANPVEVQSYQPPWKVLSDFALQSDI | |
| | | | | | DQPAFQQLVNFSEGGPGSNSTGSEVASMSSQLP | |
| | | | | | DTPNSMVASPIEA | |
| 447 | JHU1 | NIV | 1.16384410 | CELA2 | MSPILGYWKIKGLVQPTRLLLEYLEEKYEEHLYER | Full Literal Sequence- |
| | 2944 | OIPI | 3 | B | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMIRTLLLSTLVAGALS | |
| | | | | | CGVSTYAPDMSRMLGGEEARPNSWPWQVSLQY | |
| | | | | | SSNGQWYHTCGGSLIANSWVLTAAHCISSSGIYR | |
| | | | | | VMLGQHNLYVAESGSLAVSVSKIVVHKDWNSDQ | |
| | | | | | VSKGNDIALLKLANPVSLTDKIQLACLPPAGTILPN | |
| | | | | | NYPCYVTGWGRLQTNGALPDDLKQGRLLVVDYA | |
| | | | | | TCSSSGWWGSTVKTNMICAGGDGAICTCNGDSG | |
| | | | | | GPLNCQASDGRWEVHGIGSLTSVLGCNYYYKPSI | |
| | | | | | FTRVSNYNDWINSVIANN | |
| 448 | JHU1 | NIV | 1.16384410 | CELA2 | MIRTLLLSTLVAGALSCGVSTYAPDMSRMLGGEE | Core Human ORF Only |
| | 2944 | OIPI | 3 | B | ARPNSWPWQVSLQYSSNGQWYHTCGGSLIANS | |
| | | _RF | | | WVLTAAHCISSSGIYRVMLGQHNLYVAESGSLAV | |
| | | S | | | SVSKIVVHKDWNSDQVSKGNDIALLKLANPVSLT | |
| | | | | | DKIQLACLPPAGTILPNNYPCYVTGWGRLQTNGA | |
| | | | | | LPDDLKQGRLLVVDYATCSSSGVWVGSTVKTNMI | |
| | | | | | CAGGDGAICTCNGDSGGPLNCQASDGRWEVHGI | |
| | | | | | GSLTSVLGCNYYYKPSIFTRVSNYNDWINSVIANN | |
| 449 | JHU1 | NIV | 0.41212284 | MIER2 | MSPILGYWKIKGLVQPTRLLLEYLEEKYEEHLYER | Full Literal Sequence- |
| | 3073 | OIPI | 2 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAEASSLGRQSPRV | |
| | | | | | VSCLEHSLCPGEPGLQTTAVVSMGSGDHQFNLA | |
| | | | | | EILSQNYSVRGECEEASRCPDKPKEELEKDFISQ | |
| | | | | | SNDMPFDELLALYGYEASDPISDRESEGGDVAPN | |
| | | | | | LPDMTLDKEQIAKDLLSGEEEEETQSSADDLTPS | |
| | | | | | VTSHEASDLFPNRSGSRFLADEDREPGSSASSDT | |
| | | | | | EEDSLPANKCKKEIMVGPQFQADLSNLHLNRHCE | |
| | | | | | KIYENEDQLLWDPSVLPEREVEEFLYRAVKRRWH | |
| | | | | | EMAGPQLPEGEAVKDSEQALYELVKCNFNVEEA | |
| | | | | | LRRLRFNVKVIRDGLCAWSEEECRNFEHGFRVH | |
| | | | | | GKNFHLIQANKVRTRSVGECVEYYYLWKKSERY | |
| | | | | | DYFAQQTRLGRRKYVPSGTTDADQDLDGSDPDG | |
| | | | | | PGRPRPEQDTLTGMRTDPLSVDGTAGGLDEPGV | |
| | | | | | ASDGLPSSEPGPCSFQQLDESPAVPLSHRPPALA | |
| | | | | | DPASYQPAVTAPEPDASPRLAVDFALPKELPLISS | |
| | | | | | HVDLSGDPEETVAPAQVALSVTEFGLIGIGDVNPF | |
| | | | | | LAAHPTCPAPGLHSEPLSHCNVMTC | |
| 450 | JHU1 | NIV | 0.41212284 | MIER2 | MAEASSLGRQSPRVVSCLEHSLCPGEPGLQTTA | Core Human ORF Only |
| | 3073 | OIPI | 2 | | VVSMGSGDHQFNLAEILSQNYSVRGECEEASRC | |
| | | _RF | | | PDKPKEELEKDFISQSNDMPFDELLALYGYEASD | |
| | | S | | | PISDRESEGGDVAPNLPDMTLDKEQIAKDLLSGE | |
| | | | | | EEEETQSSADDLTPSVTSHEASDLFPNRSGSRFL | |
| | | | | | ADEDREPGSSASSDTEEDSLPANKCKKEIMVGP | |
| | | | | | QFQADLSNLHLNRHCEKIYENEDQLLWDPSVLPE | |
| | | | | | REVEEFLYRAVKRRWHEMAGPQLPEGEAVKDSE | |
| | | | | | QALYELVKCNFNVEEALRRLRFNVKVIRDGLCAW | |
| | | | | | SEEECRNFEHGFRVHGKNFHLIQANKVRTRSVG | |
| | | | | | ECVEYYYLWKKSERYDYFAQQTRLGRRKYVPSG | |
| | | | | | TTDADQDLDGSDPDGPGRPRPEQDTLTGMRTDP | |
| | | | | | LSVDGTAGGLDEPGVASDGLPSSEPGPCSFQQL | |
| | | | | | DESPAVPLSHRPPALADPASYQPAVTAPEPDASP | |
| | | | | | RLAVDFALPKELPLISSHVDLSGDPEETVAPAQVA | |
| | | | | | LSVTEFGLIGIGDVNPFLAAHPTCPAPGLHSEPLS | |
| | | | | | HCNVMTC | |
| 451 | JHU1 | NIV | 1.14939015 | MIER2 | MSPILGYWKIKGLVQPTRLLLEYLEEKYEEHLYER | Full Literal Sequence- |
| | 3073 | OIPI | 6 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | |
| | | | | | PKLVCFKKRIEAIPQIDKYLKSSKYIAWPLQGWQA | ORF, occasional ‘post |
| | | | | | TFGGGDHPPKSDLVPRGSPGISGGGGGILGRGS | stop’ end sequence) |
| | | | | | HHHHHHGGGITSLYKKAGTMAEASSLGRQSPRV | |
| | | | | | VSCLEHSLCPGEPGLQTTAVVSMGSGDHQFNLA | |
| | | | | | EILSQNYSVRGECEEASRCPDKPKEELEKDFISQ | |
| | | | | | SNDMPFDELLALYGYEASDPISDRESEGGDVAPN | |
| | | | | | LPDMTLDKEQIAKDLLSGEEEEETQSSADDLTPS | |
| | | | | | VTSHEASDLFPNRSGSRFLADEDREPGSSASSDT | |
| | | | | | EEDSLPANKCKKEIMVGPQFQADLSNLHLNRHCE | |
| | | | | | KIYENEDQLLWDPSVLPEREVEEFLYRAVKRRWH | |
| | | | | | EMAGPQLPEGEAVKDSEQALYELVKCNFNVEEA | |
| | | | | | LRRLRFNVKVIRDGLCAWSEEECRNFEHGFRVH | |
| | | | | | GKNFHLIQANKVRTRSVGECVEYYYLWKKSERY | |
| | | | | | DYFAQQTRLGRRKYVPSGTTDADQDLDGSDPDG | |
| | | | | | PGRPRPEQDTLTGMRTDPLSVDGTAGGLDEPGV | |
| | | | | | ASDGLPSSEPGPCSFQQLDESPAVPLSHRPPALA | |
| | | | | | DPASYQPAVTAPEPDASPRLAVDFALPKELPLISS | |
| | | | | | HVDLSGDPEETVAPAQVALSVTEFGLIGIGDVNPF | |
| | | | | | LAAHPTCPAPGLHSEPLSHCNVMTC | |
| 452 | JHU1 | NIV | 1.14939015 | MIER2 | MAEASSLGRQSPRVVSCLEHSLCPGEPGLQTTA | Core Human ORF Only |
| | 3073 | OIPI | 6 | | VVSMGSGDHQFNLAEILSQNYSVRGECEEASRC | |
| | | _RF | | | PDKPKEELEKDFISQSNDMPFDELLALYGYEASD | |
| | | S | | | PISDRESEGGDVAPNLPDMTLDKEQIAKDLLSGE | |
| | | | | | EEEETQSSADDLTPSVTSHEASDLFPNRSGSRFL | |
| | | | | | ADEDREPGSSASSDTEEDSLPANKCKKEIMVGP | |
| | | | | | QFQADLSNLHLNRHCEKIYENEDQLLWDPSVLPE | |
| | | | | | REVEEFLYRAVKRRWHEMAGPQLPEGEAVKDSE | |
| | | | | | QALYELVKCNFNVEEALRRLRFNVKVIRDGLCAW | |
| | | | | | SEEECRNFEHGFRVHGKNFHLIQANKVRTRSVG | |
| | | | | | ECVEYYYLWKKSERYDYFAQQTRLGRRKYVPSG | |
| | | | | | TTDADQDLDGSDPDGPGRPRPEQDTLTGMRTDP | |
| | | | | | LSVDGTAGGLDEPGVASDGLPSSEPGPCSFQQL | |
| | | | | | DESPAVPLSHRPPALADPASYQPAVTAPEPDASP | |
| | | | | | RLAVDFALPKELPLISSHVDLSGDPEETVAPAQVA | |
| | | | | | LSVTEFGLIGIGDVNPFLAAHPTCPAPGLHSEPLS | |
| | | | | | HCNVMTC | |
| 453 | JHU1 | NIV | 0.70601744 | RTFDC | MSPILGYWKIKGLVQPTRLLLEYLEEKYEEHLYER | Full Literal Sequence- |
| | 3412 | OIPI | 7 | 1 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGCDGGTIPKRHEL | |
| | | | | | VKGPKKVEKVDKDAELVAQWNYCTLSQEILRRPI | |
| | | | | | VACELGRLYNKDAVIEFLLDKSAEKALGKAASHIK | |
| | | | | | SIKNVTELKLSDNPAWEGDKGNTKGDKHDDLQR | |
| | | | | | ARFICPVVGLEMNGRHRFCFLRCCGCVFSERALK | |
| | | | | | EIKAEVCHTCGAAFQEDDVIVLNGTKEDVDVLKTR | |
| | | | | | MEERRLRAKLEKKTKKPKAAESVSKPDVSEEAPG | |
| | | | | | PSKVKTGKPEEASLDSREKKTNLAPKSTAMNESS | |
| | | | | | SGKAGKPPCGATKRSIADSEESEAYKSLFTTHSS | |
| | | | | | AKRSKEESAHWVTHTSYCF | |
| 454 | JHU1 | NIV | 0.70601744 | RTFDC | MGCDGGTIPKRHELVKGPKKVEKVDKDAELVAQ | Core Human ORF Only |
| | 3412 | OIPI | 7 | 1 | WNYCTLSQEILRRPIVACELGRLYNKDAVIEFLLD | |
| | | _RF | | | KSAEKALGKAASHIKSIKNVTELKLSDNPAWEGDK | |
| | | S | | | GNTKGDKHDDLQRARFICPVVGLEMNGRHRFCF | |
| | | | | | LRCCGCVFSERALKEIKAEVCHTCGAAFQEDDVI | |
| | | | | | VLNGTKEDVDVLKTRMEERRLRAKLEKKTKKPKA | |
| | | | | | AESVSKPDVSEEAPGPSKVKTGKPEEASLDSREK | |
| | | | | | KTNLAPKSTAMNESSSGKAGKPPCGATKRSIADS | |
| | | | | | EESEAYKSLFTTHSSAKRSKEESAHVWTHTSYCF | |
| 455 | JHU1 | NIV | 1.55493275 | LOC44 | MSPILGYWKIKGLVQPTRLLLEYLEEKYEEHLYER | Full Literal Sequence- |
| | 5058 | OIPI | 6 | 0337 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSCVLSPILVLHRPL | |
| | | | | | KSKRGDLVFFLCVLWSSSILDRPCMFRKRGSTLC | |
| | | | | | PPKMMAHLSRPGWRLSYQSFCCLGGRLPAADV | |
| | | | | | CPRLSSLQPLSSICRTLGHSCQCFPSVLTFHLSRR | |
| | | | | | LGFHASGPNREMVTQTLRSWCHNPSQQLAGHLS | |
| | | | | | AVACQHAGSSTDGGC | |
| 456 | JHU1 | NIV | 1.55493275 | LOC44 | MSCVLSPILVLHRPLKSKRGDLVFFLCVLWSSSIL | Core Human ORF Only |
| | 5058 | OIPI | 6 | 0337 | DRPCMFRKRGSTLCPPKMMAHLSRPGWRLSYQ | |
| | | _RF | | | SFCCLGGRLPAADVCPRLSSLQPLSSICRTLGHS | |
| | | S | | | CQCFPSVLTFHLSRRLGFHASGPNREMVTQTLR | |
| | | | | | SWCHNPSQQLAGHLSAVACQHAGSSTDGGC | |
| 457 | JHU1 | NIV | 0.75117644 | SUSD3 | MSPILGYWKIKGLVQPTRLLLEYLEEKYEEHLYER | Full Literal Sequence- |
| | 5566 | OIPI | 9 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSMAFLTCCLLKCVK | |
| | | | | | KSKRRRSNRSAQLWSQLKDEDLETVQAAYLGLK | |
| | | | | | HFNKPVSGPSQAHDNHSFTTDHGESTSKLASVT | |
| | | | | | RSVDKDPGIPRALSLSGSSSSPQAQVMVHMANP | |
| | | | | | RQPLPASGLATGMPQQPAAYALG | |
| 458 | JHU1 | NIV | 0.75117644 | SUSD3 | MSMAFLTCCLLKCVKKSKRRRSNRSAQLWSQLK | Core Human ORF Only |
| | 5566 | OIPI | 9 | | DEDLETVQAAYLGLKHFNKPVSGPSQAHDNHSF | |
| | | _RF | | | TTDHGESTSKLASVTRSVDKDPGIPRALSLSGSS | |
| | | S | | | SSPQAQVMVHMANPRQPLPASGLATGMPQQPA | |
| | | | | | AYALG | |
| 459 | JHU1 | NIV | 0.84629121 | MESP2 | MSPILGYWKIKGLVQPTRLLLEYLEEKYEEHLYER | Full Literal Sequence- |
| | 6751 | OIPI | 4 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAQSPPPQSLLGHD | |
| | | | | | HWIFAQGWGWAGHWDSTSPASSSDSSGSCPCD | |
| | | | | | GARGLPQPQPPSCSSRAAEAAATTPRRARTGPA | |
| | | | | | GGQRQSASEREKLRMRTLARALHELRRFLPPSLA | |
| | | | | | PAGQSLTKIETLRLAIRYIGHLSAVLGLSEESLQCR | |
| | | | | | RRQRGDAGSPWGCPLCPDRGPAEAQTQAEGQ | |
| | | | | | GQGQGQGQGQGQGQGQGQGQGQGQGRRPGL | |
| | | | | | VSAVLAEASWGSPSACPGAQAAPERLGRGVHDT | |
| | | | | | DPWATPPYCPKIQSPPYSSQGTTSDASLWTPPQ | |
| | | | | | GCPWTQSSPEPRNPPVPWTAAPATLELAAVYQG | |
| | | | | | LSVSPEPCLSLGAPSLLPHPSCQRLQPQTPGRC | |
| | | | | | WSHSAEVVPNSEDQGPGAAFQLSEASPPQSSGL | |
| | | | | | RFSGCPELWQEDLEGARLGIFY | |
| 460 | JHU1 | NIV | 0.84629121 | MESP2 | MAQSPPPQSLLGHDHWIFAQGWGWAGHWDSTS | Core Human ORF Only |
| | 6751 | OIPI | 4 | | PASSSDSSGSCPCDGARGLPQPQPPSCSSRAAE | |
| | | _RF | | | AAATTPRRARTGPAGGQRQSASEREKLRMRTLA | |
| | | S | | | RALHELRRFLPPSLAPAGQSLTKIETLRLAIRYIGH | |
| | | | | | LSAVLGLSEESLQCRRRQRGDAGSPWGCPLCPD | |
| | | | | | RGPAEAQTQAEGQGQGQGQGQGQGQGQGQG | |
| | | | | | QGQGQGQGRRPGLVSAVLAEASWGSPSACPGA | |
| | | | | | QAAPERLGRGVHDTDPWATPPYCPKIQSPPYSS | |
| | | | | | QGTTSDASLWTPPQGCPWTQSSPEPRNPPVPW | |
| | | | | | TAAPATLELAAVYQGLSVSPEPCLSLGAPSLLPHP | |
| | | | | | SCQRLQPQTPGRCWSHSAEVVPNSEDQGPGAA | |
| | | | | | FQLSEASPPQSSGLRFSGCPELWQEDLEGARLGI | |
| | | | | | FY | |
| 461 | JHU1 | NIV | 1.64891985 | ATP1B | MSPILGYWKIKGLVQPTRLLLEYLEEKYEEHLYER | Full Literal Sequence- |
| | 7021 | OIPI | 6 | 4 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMRRQLRSRRAPSFP | |
| | | | | | YSYRYRLDDPDEANQNYLADEEEEAEEEARVTV | |
| | | | | | VPKSEEEEEEEEKEEEEEEEKEEEEGQGQPTGN | |
| | | | | | AVWVQKLQIMSEYLWDPERRMFLARTGQSWSLILLI | |
| | | | | | YFFFYASLAAVITLCMYTLFLTISPYIPTFTERVKP | |
| | | | | | PGVMIRPFAHSLNFNFNVSEPDTWQHYVISLNGF | |
| | | | | | LQGYNDSLQEEMNVDCPPGQYFIQDGNEDEDKK | |
| | | | | | ACQFKRSFLKNCSGLEDPTFGYSTGQPCILLKMN | |
| | | | | | RIVGFRPELGDPVKVSCKVQRGDENDIRSISYYPE | |
| | | | | | SASFDLRYYPYYGKLTHVNYTSPLVAMHFTDVVK | |
| | | | | | NQAVPVQCQLKGKGVINDVINDRFVGRVIFTLNIE | |
| | | | | | TNPAFLYKVVKASS | |
| 462 | JHU1 | NIV | 1.64891985 | ATP1B | MRRQLRSRRAPSFPYSYRYRLDDPDEANQNYLA | Core Human ORF Only |
| | 7021 | OIPI | 6 | 4 | DEEEEAEEEARVTVVPKSEEEEEEEEKEEEEEEE | |
| | | _RF | | | KEEEEGQGQPTGNAWWQKLQIMSEYLWDPERR | |
| | | S | | | MFLARTGQSWSLILLIYFFFYASLAAVITLCMYTLF | |
| | | | | | LTISPYIPTFTERVKPPGVMIRPFAHSLNFNFNVSE | |
| | | | | | PDTWQHYVISLNGFLQGYNDSLQEEMNVDCPPG | |
| | | | | | QYFIQDGNEDEDKKACQFKRSFLKNCSGLEDPTF | |
| | | | | | GYSTGQPCILLKMNRIVGFRPELGDPVKVSCKVQ | |
| | | | | | RGDENDIRSISYYPESASFDLRYYPYYGKLTHVNY | |
| | | | | | TSPLVAMHFTDVVKNQAVPVQCQLKGKGVINDVI | |
| | | | | | NDRFVGRVIFTLNIET | |
| 463 | JHU1 | NIV | 0.65536566 | C1or61 | MSPILGYWKIKGLVQPTRLLLEYLEEKYEEHLYER | Full Literal Sequence- |
| | 7609 | OIPI | 2 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMFLTEDLITFNLRNFL | |
| | | | | | LFQLWESSFSPGAGGFCTTLPPSFLRVDDRATSS | |
| | | | | | TTDSSRAPSSPRPPGSTSHCGISTRCTERCLCVL | |
| | | | | | PLRTSQVPDVMAPQHDQEKFHDLAYSCLGKSFS | |
| | | | | | MSNQDLYGYSTSSLALGLAWLSWETKKKNVLHL | |
| | | | | | VGLDSLNPAFLYKVVKASS | |
| 464 | JHU1 | NIV | 0.65536566 | C1or61 | MFLTEDLITFNLRNFLLFQLWESSFSPGAGGFCTT | Core Human ORF Only |
| | 7609 | OIPI | 2 | | LPPSFLRVDDRATSSTTDSSRAPSSPRPPGSTSH | |
| | | _RF | | | CGISTRCTERCLCVLPLRTSQVPDVMAPQHDQEK | |
| | | S | | | FHDLAYSCLGKSFSMSNQDLYGYSTSSLALGLA | |
| | | | | | WLSWETKKKNVLHLVGLDSL | |
| 465 | JHU1 | NIV | 0.14744319 | TTLL13 | MSPILGYWKIKGLVQPTRLLLEYLEEKYEEHLYER | Full Literal Sequence- |
| | 7959 | OIPI | 9 | P | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEPSTCRTMESEED | |
| | | | | | YVEEKESEKCVKEGVTNPSNSSQQALLKADYKAL | |
| | | | | | KNGVPSPIMATKIPKKVIAPVDTGDLEAGRRKRRR | |
| | | | | | KRRSLAINLTNCKYESVRRAAQMCGLKEVGEDEE | |
| | | | | | WTLYWTDCAVSLERVMDMKRFQKINHFPGMTEI | |
| | | | | | CRKDLLARNLNRMYKLYPSEYNIFPRTWCLPADY | |
| | | | | | GDFQSYGRQRKARTYICKPDSGCQGRGIFITRNP | |
| | | | | | REIKPGEHMICQQYISKPLLIDGFKFDMRVYVLITS | |
| | | | | | CDPLRIFTYEEGLARFATTPYMEPSHNNLDNVCM | |
| | | | | | HLTNYAINKHNENFVRDGAVGSKRKLSTLNIWLQ | |
| | | | | | EHSYNPGELWGDIEDIIIKTIISAHSVLRHNYRTCF | |
| | | | | | PQYLNGGTCACFEILGFDILLDHKLKPWLLEVNHS | |
| | | | | | PSFTTDSCLDQEVKDALLCDAMTLVNLRGCDKRK | |
| | | | | | VMEEDKRRVKERLFQCYRQPRESRCARCLACVN | |
| | | | | | PAFLYKVVKASS | |
| 466 | JHU1 | NIV | 0.14744319 | TTLL13 | MEPSTCRTMESEEDYVEEKESEKCVKEGVTNPS | Core Human ORF Only |
| | 7959 | OIPI | 9 | P | NSSQQALLKADYKALKNGVPSPIMATKIPKKVIAP | |
| | | _RF | | | VDTGDLEAGRRKRRRKRRSLAINLTNCKYESVRR | |
| | | S | | | AAQMCGLKEVGEDEEWTLYWTDCAVSLERVMD | |
| | | | | | MKRFQKINHFPGMTEICRKDLLARNLNRMYKLYP | |
| | | | | | SEYNIFPRTWCLPADYGDFQSYGRQRKARTYICK | |
| | | | | | PDSGCQGRGIFITRNPREIKPGEHMICQQYISKPL | |
| | | | | | LIDGFKFDMRVYVLITSCDPLRIFTYEEGLARFATT | |
| | | | | | PYMEPSHNNLDNVCMHLTNYAINKHNENFVRDG | |
| | | | | | AVGSKRKLSTLNIWLQEHSYNPGELWGDIEDIIIKT | |
| | | | | | IISAHSVLRHNYRTCFPQYLNGGTCACFEILGFDIL | |
| | | | | | LDHKLKPWLLEVNHSPSFTTDSCLDQEVKDALLC | |
| | | | | | DAMTLVNLRGCDKRKVMEEDKRRVKERLFQCYR | |
| | | | | | QPRESRCARCLACV | |
| 467 | JHU1 | NIV | 1.30618727 | TTL | MSPILGYWKIKGLVQPTRLLLEYLEEKYEEHLYER | Full Literal Sequence- |
| | 8516 | OIPI | 2 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMYTFVVRDENSSVY | |
| | | | | | AEVSRLLLATGHWKRLRRDNPRFNLMLGERNRL | |
| | | | | | PFGRLGHEPGLVQLVNYYRGADKLCRKASLVKLI | |
| | | | | | KTSPELAESCTWFPESYVIYPTNLKTPVAPAQNGI | |
| | | | | | QPPISNSRTDEREFFLASYNRKKEDGEGNVWIAK | |
| | | | | | SSAGAKGEGILISSEASELLDFIDNQGQVHVIQKYL | |
| | | | | | EHPLLLEPGHRKFDIRSWLVDHQYNIYLYREGVL | |
| | | | | | RTASEPYHVDNFQDKTCHLTNHCIQKEYSKNYGK | |
| | | | | | YEEGNEMFFKEFNQYLTSALNITLESSILLQIKHIIR | |
| | | | | | NCLLSVEPAISTKHLPYQSFQLFGFDFMVDEELKV | |
| | | | | | WLIEVNGAPACAQKLYAELCQGIVDIAISSVFPPP | |
| | | | | | DVEQPQTQPAAFIKLNPAFLYKVVKASS | |
| 468 | JHU1 | NIV | 1.30618727 | TTL | MYTFVVRDENSSVYAEVSRLLLATGHWKRLRRD | Core Human ORF Only |
| | 8516 | OIPI | 2 | | NPRFNLMLGERNRLPFGRLGHEPGLVQLVNYYR | |
| | | _RF | | | GADKLCRKASLVKLIKTSPELAESCTWFPESYVIY | |
| | | S | | | PTNLKTPVAPAQNGIQPPISNSRTDEREFFLASYN | |
| | | | | | RKKEDGEGNVWIAKSSAGAKGEGILISSEASELLD | |
| | | | | | FIDNQGQVHVIQKYLEHPLLLEPGHRKFDIRSWVL | |
| | | | | | VDHQYNIYLYREGVLRTASEPYHVDNFQDKTCHL | |
| | | | | | TNHCIQKEYSKNYGKYEEGNEMFFKEFNQYLTSA | |
| | | | | | LNITLESSILLQIKHIIRNCLLSVEPAISTKHLPYQ | |
| | | | | | SFQLFGFDFMVDEELKVWLIEVNGAPACAQKLYAEL | |
| | | | | | CQGIVDIAISSVFPPPDVEQPQTQPAAFIKL | |
| 469 | JHU1 | NIV | 0.23395662 | CDKL2 | MSPILGYWKIKGLVQPTRLLLEYLEEKYEEHLYER | Full Literal Sequence- |
| | 9362 | OIPI | 5 | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEKYENLGLVGEGS | |
| | | | | | YGMVMKCRNKDTGRIVAIKKFLESDDDKMVKKIA | |
| | | | | | MREIKLLKQLRHENLVNLLEVCKKKKRWYLVFEF | |
| | | | | | VDHTILDDLELFPNGLDYQVVQKYLFQIINGIGFCH | |
| | | | | | SHNIIHRDIKPENILVSQSGVVKLCDFGFARTLAAP | |
| | | | | | GEVYTDYVATRWYRAPELLVGDVKYGKAVDVWA | |
| | | | | | IGCLVTEMFMGEPLFPGDSDIDQLYHIMMCLGNLI | |
| | | | | | PRHQELFNKNPVFAGVRLPEIKEREPLERRYPKL | |
| | | | | | SEVVIDLAKKCLHIDPDKRPFCAELLHHDFFQMDG | |
| | | | | | FAERFSQELQLKVQKDARNVSLSKKSQNRKKEK | |
| | | | | | EKDDSLVEERKTLVVQDTNADPKIKDYKLFKIKGS | |
| | | | | | KIDGEKAEKGNRASNASCLHDSRTSHNKIVPSTS | |
| | | | | | LKDCSNVSVDHTRNPSVAIPPLTHNLSAVAPSINS | |
| | | | | | GMGTETIPIQGYRVDEKTKKCSIPFVKPNRHSPS | |
| | | | | | GIYNINVTTLVSGPPLSDDSGADLPQMEHQH | |
| 470 | JHU1 | NIV | 0.23395662 | CDKL2 | MEKYENLGLVGEGSYGMVMKCRNKDTGRIVAIK | Core Human ORF Only |
| | 9362 | OIPI | 5 | | KFLESDDDKMVKKIAMREIKLLKQLRHENLVNLLE | |
| | | _RF | | | VCKKKKRWYLVFEFVDHTILDDLELFPNGLDYQV | |
| | | S | | | VQKYLFQIINGIGFCHSHNIIHRDIKPENILVSQSGV | |
| | | | | | VKLCDFGFARTLAAPGEVYTDYVATRWYRAPELL | |
| | | | | | VGDVKYGKAVDVWAIGCLVTEMFMGEPLFPGDS | |
| | | | | | DIDQLYHIMMCLGNLIPRHQELFNKNPVFAGVRLP | |
| | | | | | EIKEREPLERRYPKLSEVVIDLAKKCLHIDPDKRPF | |
| | | | | | CAELLHHDFFQMDGFAERFSQELQLKVQKDARN | |
| | | | | | VSLSKKSQNRKKEKEKDDSLVEERKTLVVQDTNA | |
| | | | | | DPKIKDYKLFKIKGSKIDGEKAEKGNRASNASCLH | |
| | | | | | DSRTSHNKIVPSTSLKDCSNVSVDHTRNPSVAIPP | |
| | | | | | LTHNLSAVAPSINSGMGTETIPIQGYRVDEKTKKC | |
| | | | | | SIPFVKPNRHSPSGIYNINVTTLVSGPPLSDDSGA | |
| | | | | | DLPQMEHQH | |
| 471 | JHU1 | NIV | 0.083728268 | Cebpa | MSPILGYWKIKGLVQPTRLLLEYLEEKYEEHLYER | Full Literal Sequence- |
| | 9619 | OIPI | | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMESADFYEVEPRPP | |
| | | | | | MSSHLQSPPHAPSNAAFGFPRGAGPAPPPAPPA | |
| | | | | | APEPLGGICEHETSIDISAYIDPAAFNDEFLADLFQ | |
| | | | | | HSRQQEKAKAAAGPAGGGGDFDYPGAPAGPGG | |
| | | | | | AVMSAGAHGPPPGYGCAAAGYLDGRLEPLYERV | |
| | | | | | GAPALRPLVIKQEPREEDEAKQLALAGLFPYQPP | |
| | | | | | PPPPPPHPHASPAHLAAPHLQFQIAHCGQTTMHL | |
| | | | | | QPGHPTPPPTPVPSPHAAPALGAAGLPGPGSAL | |
| | | | | | KGLAGAHPDLRTGGGGGGSGAGAGKAKKSVDK | |
| | | | | | NSNEYRVRRERNNIAVRKSRDKAKQRNVETQQK | |
| | | | | | VLELTSDNDRLRKRVEQLSRELDTLRGIFRQLPES | |
| | | | | | SLVKAMGNCANPAFLYKVVKASS | |
| 472 | JHU1 | NIV | 0.083728268 | Cebpa | MESADFYEVEPRPPMSSHLQSPPHAPSNAAFGF | Core Human ORF Only |
| | 9619 | OIPI | | | PRGAGPAPPPAPPAAPEPLGGICEHETSIDISAYI | |
| | | _RF | | | DPAAFNDEFLADLFQHSRQQEKAKAAAGPAGGG | |
| | | S | | | GDFDYPGAPAGPGGAVMSAGAHGPPPGYGCAA | |
| | | | | | AGYLDGRLEPLYERVGAPALRPLVIKQEPREEDE | |
| | | | | | AKQLALAGLFPYQPPPPPPPPHPHASPAHLAAPH | |
| | | | | | LQFQIAHCGQTTMHLQPGHPTPPPTPVPSPHAAP | |
| | | | | | ALGAAGLPGPGSALKGLAGAHPDLRTGGGGGGS | |
| | | | | | GAGAGKAKKSVDKNSNEYRVRRERNNIAVRKSR | |
| | | | | | DKAKQRNVETQQKVLELTSDNDRLRKRVEQLSR | |
| | | | | | ELDTLRGIFRQLPESSLVKAMGNCA | |
| 473 | JHU1 | NIV | 0.65797989 | ZNF64 | MSPILGYWKIKGLVQPTRLLLEYLEEKYEEHLYER | Full Literal Sequence- |
| | 9908 | OIPI | 4 | 6 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEDTPPSLSCSDCQ | |
| | | | | | RHFPSLPELSRHRELLHPSPNQDSEEADSIPRPY | |
| | | | | | RCQQCGRGYRHPGSLVNHRRTHETGLFPCTTC | |
| | | | | | GKDFSNPMALKSHMRTHAPEGRRRHRPPRPKEA | |
| | | | | | TPHLQGETVSTDSWGQRLGSSEGWENQTKHTE | |
| | | | | | ETPDCESVPDPRAASGTWEDLPTRQREGLASHP | |
| | | | | | GPEDGADGWGPSTNSARAPPLPIPASSLLSNLEQ | |
| | | | | | YLAESVVNFTGGQEPTQSPPAEEERRYKCSQCG | |
| | | | | | KTYKHAGSLTNHRQSHTLGIYPCAICFKEFSNLMA | |
| | | | | | LKNHSRLHAQYRPYHCPHCPRVFRLPRELLEHQ | |
| | | | | | QSHEGERQEPRWEGKGMPTTNGHTDESSQDQL | |
| | | | | | PSAQMLNGSAELSTSGELEDSGLEEYRPFRCGD | |
| | | | | | CGRTYRHAGSLINHRKSHQTGVYPCSLCSKQLFN | |
| | | | | | AAALKNHVRAHHRPRQGVGENGQPSVPPAPLLL | |
| | | | | | AETTHKEEEDPTTTLDHRPYKCSECGRAYRHRG | |
| | | | | | SLVNHRHSHRTGEYQCSLCPRKYPNLMALRNHV | |
| | | | | | RVHCKAARRSADIGAEGAPSHLKVELPPDPVEAE | |
| | | | | | AAPHTDQDHVCKHEEEATDITPAADKTAAHICSIC | |
| | | | | | GLLFEDAESLERHGLTHGAGEKENSRTETTMSPP | |
| | | | | | RAFACRDCGKSYRHSGSLINHRQTHQTGDFSCG | |
| | | | | | ACAKHFHTMAAMKNHLRRHSRRRSRRHRKRAG | |
| | | | | | GASGGREAKLLAAESWTRELEDNEGLESPQDPS | |
| | | | | | GESPHGAEGNLESDGDCLQAESEGDKCGLERDE | |
| | | | | | THFQGDKESGGTGEGLERKDASLLDNLDIPGEEG | |
| | | | | | GGTHFCDSLTGVDEDQKPATGQPNSSSHSANAV | |
| | | | | | TGWQAGAAHTCSDCGHSFPHATGLLSHRPCHPP | |
| | | | | | GIYQCSLCPKEFDSLPALRSHFQNHRPGEATSAQ | |
| | | | | | PFLCCLCGMIFPGRAGYRLHRRQAHSSSGMTEG | |
| | | | | | SEEEGEEEGVAEAAPARSPPLQLSEAELLNQLQR | |
| | | | | | EVEALDSAGYGHICGCCGQTYDDLGSLERHHQS | |
| | | | | | QSSGTTADKAPSPLGVAGDAMEMVVDSVLEDIV | |
| | | | | | NSVSGEGGDAKSQEGAGTPLGDSLCIQGGESLL | |
| | | | | | EAQPRPFRCNQCGKTYRHGGSLVNHRKIHQTGD | |
| | | | | | FLCPVCSRCYPNLAAYRNHLRNHPRCKGSEPQV | |
| | | | | | GPIPEAAGSSELQVGPIPEGGSNKPQHMAEEGP | |
| | | | | | GQAEVEKLQEELKVEPLEEVARVKEEVWEETTVK | |
| | | | | | GEEIEPRLETAEKGCQTEASSERPFSCEVCGRSY | |
| | | | | | KHAGSLINHRQSHQTGHFGCQACSKGFSNLMSL | |
| | | | | | KNHRRIHADPRRFRCSECGKAFRLRKQLASHQR | |
| | | | | | VHMERRGGGGTRKATREDRPFRCGQCGRTYRH | |
| | | | | | AGSLLNHRRSHETGQYSCPTCPKTYSNRMALKD | |
| | | | | | HQRLHSENRRRRAGRSRRTAVRCALCGRSFPG | |
| | | | | | RGSLERHLREHEETEREPANGQGGLDGTAASEA | |
| | | | | | NLTGSQGLETQLGGAEPVPHLEDGVPRPGERSQ | |
| | | | | | SPIRAASSEAPEPLSWGAGKAGGWPVGGGLGN | |
| | | | | | HSGGVWPQFLTRSEEPEDSVHRSPCHAGDCQL | |
| | | | | | NGPTLSHMDSWDNRDNSSQLQPGSHSSCSQCG | |
| | | | | | KTYCQSGSLLNHNTNKTDRHYCLLCSKEFLNPVA | |
| | | | | | TKSHSHNHIDAQTFACPDCGKAFESHQELASHLQ | |
| | | | | | AHARGHSQVPAQMEEARDPKAGTGEDQVVLPG | |
| | | | | | QGKAQEAPSETPRGPGESVERARGGQAVTSMA | |
| | | | | | AEDKERPFRCTQCGRSYRHAGSLLNHQKAHTTG | |
| | | | | | LYPCSLCPKLLPNLLSLKNHSRTHTDPKRHCCSIC | |
| | | | | | GKAFRTAARLEGHGRVHAPREGPFTCPHCPRHF | |
| | | | | | RRRISFVQHQQQHQEEWTVAGSGAPVAPVTGR | |
| | | | | | GDLPLPPPPTPTTPLLDPSPQWPADLSFSLNPAF | |
| | | | | | LYKVVKASS | |
| 474 | JHU1 | NIV | 0.657979894 | ZNF64 | MEDTPPSLSCSDCQRHFPSLPELSRHRELLHPSP | Core Human ORF Only |
| | 9908 | OIPI | | 6 | NQDSEEADSIPRPYRCQQCGRGYRHPGSLVNHR | |
| | | _RF | | | RTHETGLFPCTTCGKDFSNPMALKSHMRTHAPE | |
| | | S | | | GRRRHRPPRPKEATPHLQGETVSTDSWGQRLG | |
| | | | | | SSEGWENQTKHTEETPDCESVPDPRAASGTWE | |
| | | | | | DLPTRQREGLASHPGPEDGADGWGPSTNSARA | |
| | | | | | PPLPIPASSLLSNLEQYLAESVVNFTGGQEPTQSP | |
| | | | | | PAEEERRYKCSQCGKTYKHAGSLTNHRQSHTLGI | |
| | | | | | YPCAICFKEFSNLMALKNHSRLHAQYRPYHCPHC | |
| | | | | | PRVFRLPRELLEHQQSHEGERQEPRWEGKGMP | |
| | | | | | TTNGHTDESSQDQLPSAQMLNGSAELSTSGELE | |
| | | | | | DSGLEEYRPFRCGDCGRTYRHAGSLINHRKSHQ | |
| | | | | | TGVYPCSLCSKQLFNAAALKNHVRAHHRPRQGV | |
| | | | | | GENGQPSVPPAPLLLAETTHKEEEDPTTTLDHRP | |
| | | | | | YKCSECGRAYRHRGSLVNHRHSHRTGEYQCSLC | |
| | | | | | PRKYPNLMALRNHVRVHCKAARRSADIGAEGAP | |
| | | | | | SHLKVELPPDPVEAEAAPHTDQDHVCKHEEEATD | |
| | | | | | ITPAADKTAAHICSICGLLFEDAESLERHGLTHGA | |
| | | | | | GEKENSRTETTMSPPRAFACRDCGKSYRHSGSLI | |
| | | | | | NHRQTHQTGDFSCGACAKHFHTMAAMKNHLRR | |
| | | | | | HSRRRSRRHRKRAGGASGGREAKLLAAESWTR | |
| | | | | | ELEDNEGLESPQDPSGESPHGAEGNLESDGDCL | |
| | | | | | QAESEGDKCGLERDETHFQGDKESGGTGEGLE | |
| | | | | | RKDASLLDNLDIPGEEGGGTHFCDSLTGVDEDQK | |
| | | | | | PATGQPNSSSHSANAVTGWQAGAAHTCSDCGH | |
| | | | | | SFPHATGLLSHRPCHPPGIYQCSLCPKEFDSLPA | |
| | | | | | LRSHFQNHRPGEATSAQPFLCCLCGMIFPGRAG | |
| | | | | | YRLHRRQAHSSSGMTEGSEEEGEEEGVAEAAPA | |
| | | | | | RSPPLQLSEAELLNQLQREVEALDSAGYGHICGC | |
| | | | | | CGQTYDDLGSLERHHQSQSSGTTADKAPSPLGV | |
| | | | | | AGDAMEMVVDSVLEDIVNSVSGEGGDAKSQEGA | |
| | | | | | GTPLGDSLCIQGGESLLEAQPRPFRCNQCGKTY | |
| | | | | | RHGGSLVNHRKIHQTGDFLCPVCSRCYPNLAAY | |
| | | | | | RNHLRNHPRCKGSEPQVGPIPEAAGSSELQVGPI | |
| | | | | | PEGGSNKPQHMAEEGPGQAEVEKLQEELKVEPL | |
| | | | | | EEVARVKEEVWEETTVKGEEIEPRLETAEKGCQT | |
| | | | | | EASSERPFSCEVCGRSYKHAGSLINHRQSHQTG | |
| | | | | | HFGCQACSKGFSNLMSLKNHRRIHADPRRFRCS | |
| | | | | | ECGKAFRLRKQLASHQRVHMERRGGGGTRKAT | |
| | | | | | REDRPFRCGQCGRTYRHAGSLLNHRRSHETGQ | |
| | | | | | YSCPTCPKTYSNRMALKDHQRLHSENRRRRAGR | |
| | | | | | SRRTAVRCALCGRSFPGRGSLERHLREHEETER | |
| | | | | | EPANGQGGLDGTAASEANLTGSQGLETQLGGAE | |
| | | | | | PVPHLEDGVPRPGERSQSPIRAASSEAPEPLSW | |
| | | | | | GAGKAGGWPVGGGLGNHSGGWVPQFLTRSEEP | |
| | | | | | EDSVHRSPCHAGDCQLNGPTLSHMDSWDNRDN | |
| | | | | | SSQLQPGSHSSCSQCGKTYCQSGSLLNHNTNKT | |
| | | | | | DRHYCLLCSKEFLNPVATKSHSHNHIDAQTFACP | |
| | | | | | DCGKAFESHQELASHLQAHARGHSQVPAQMEEA | |
| | | | | | RDPKAGTGEDQVVLPGQGKAQEAPSETPRGPG | |
| | | | | | ESVERARGGQAVTSMAAEDKERPFRCTQCGRS | |
| | | | | | YRHAGSLLNHQKAHTTGLYPCSLCPKLLPNLLSL | |
| | | | | | KNHSRTHTDPKRHCCSICGKAFRTAARLEGHGR | |
| | | | | | VHAPREGPFTCPHCPRHFRRRISFVQHQQQHQE | |
| | | | | | EWTVAGSGAPVAPVTGRGDLPLPPPPTPTTPLLD | |
| | | | | | PSPQWPADLSFSL | |
| 475 | JHU1 | NIV | 0.585492991 | Sebox | MSPILGYWKIKGLVQPTRLLLEYLEEKYEEHLYER | Full Literal Sequence- |
| | 9981 | OIPI | | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASPVEASPGCASG | |
| | | | | | LGPHRRKRTTFSVGQLVELERVFAARPYPDISTR | |
| | | | | | EHLAQVTHLPEAKIQVWFQNRRAKRIKDRKPGAL | |
| | | | | | NSRLELPPNSCSLPDTPQLPWDPGTSSHPLHPTS | |
| | | | | | SAQYTSACPPQTSCLGPILGPGQSWSGAKVAAP | |
| | | | | | WGTSGASGIHSSLEQIVPQTSLGNLSDLIYTSAIVT | |
| | | | | | NVDHFNPAFLYKVVKASS | |
| 476 | JHU1 | NIV | 0.585492991 | Sebox | MASPVEASPGCASGLGPHRRKRTTFSVGQLVEL | Core Human ORF Only |
| | 9981 | OIPI | | | ERVFAARPYPDISTREHLAQVTHLPEAKIQVWFQ | |
| | | _RF | | | NRRAKRIKDRKPGALNSRLELPPNSCSLPDTPQL | |
| | | S | | | PWDPGTSSHPLHPTSSAQYTSACPPQTSCLGPIL | |
| | | | | | GPGQSWSGAKVAAPWGTSGASGIHSSLEQIVPQ | |
| | | | | | TSLGNLSDLIYTSAIVTNVDHF | |
| 477 | JHU2 | NIV | 0.647754944 | TCF19 | MSPILGYWKIKGLVQPTRLLLEYLEEKYEEHLYER | Full Literal Sequence- |
| | 4009 | OIPI | | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLPCFQLLRIGGGRG | |
| | | | | | GDLYTFHPPAGAGCTYRLGHRADLCDVALRPQQ | |
| | | | | | EPGLISGIHAELHAEPRGDDWRVSLEDHSSQGTL | |
| | | | | | VNNVRLPRGHRLELSDGDLLTFGPEGPPGTSPSE | |
| | | | | | FYFMFQQVRVKPQDFAAITIPRSRGEARVGAGFR | |
| | | | | | PMLPSQGAPQRPLSTFSPAPKATLILNSIGSLSKL | |
| | | | | | RPQPLTFSPSWGGPKSLPVPAPPGEVGTTPSAP | |
| | | | | | PQRNRRKSVHRVLAELDDESEPLENPPPVLMEP | |
| | | | | | RKKLRVDKAPLTPTGNRRGRPRKYPVSAPMAPP | |
| | | | | | AVGGGEPCAAPCCCLPQEETVAWVQCDGCDVW | |
| | | | | | FHVACVGCSIQAAREADFRCPGCRAGIQTNPAFL | |
| | | | | | YKVVKASS | |
| 478 | JHU2 | NIV | 0.647754944 | TCF19 | MLPCFQLLRIGGGRGGDLYTFHPPAGAGCTYRL | Core Human ORF Only |
| | 4009 | OIPI | | | GHRADLCDVALRPQQEPGLISGIHAELHAEPRGD | |
| | | _RF | | | DWRVSLEDHSSQGTLVNNVRLPRGHRLELSDGD | |
| | | S | | | LLTFGPEGPPGTSPSEFYFMFQQVRVKPQDFAAI | |
| | | | | | TIPRSRGEARVGAGFRPMLPSQGAPQRPLSTFSP | |
| | | | | | APKATLILNSIGSLSKLRPQPLTFSPSWGGPKSLP | |
| | | | | | VPAPPGEVGTTPSAPPQRNRRKSVHRVLAELDD | |
| | | | | | ESEPLENPPPVLMEPRKKLRVDKAPLTPTGNRRG | |
| | | | | | RPRKYPVSAPMAPPAVGGGEPCAAPCCCLPQEE | |
| | | | | | TVAWWVQCDGCDVWFHVACVGCSIQAAREADFR | |
| | | | | | CPGCRAGIQT | |
| 479 | JHU2 | NIV | 1.9586815 | C3orf58 | MSPILGYWKIKGLVQPTRLLLEYLEEKYEEHLYER | Full Literal Sequence- |
| | 6452 | OIPI | | | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMWRLVPPKLGRLSR | |
| | | | | | SLKLAALGSLLVLMVLHSPSLLASWQRNELTDRR | |
| | | | | | FLQLNKCPACFGTSWCRRFLNGQVVFEAWGRLR | |
| | | | | | LLDFLNVKNVYFAQYGEPREGGRRRVVLKRLGS | |
| | | | | | QRELAQLDQSICKRATGRPRCDLLQAMPRTEFAR | |
| | | | | | LNGDVRLLTPEAVEGWSDLVHCPSQRLLDRLVR | |
| | | | | | RYAETKDSGSFLLRNLKDSERMQLLLTLAFNPEP | |
| | | | | | LVLQSFPSDEGWPFAKYLGACGRMVAVNYVGEE | |
| | | | | | LWSYFNAPWEKRVDLAWQLMEIAEQLTNNDFEF | |
| | | | | | ALYLLDVSFDNFAVGPRDGKVIIVDAENVLVADKR | |
| | | | | | LIRQNKPENWDVWYESKFDDCDKEACLSFSKEIL | |
| | | | | | CARATVDHNYYAVCQNLLSRHATWRGTSGGLLH | |
| | | | | | DPPSEIAKDGRLEALLDECANPKKRYGRFQAAKE | |
| | | | | | LREYLAQLSNNVRNPAFLYKVVKASS | |
| 480 | JHU2 | NIV | 1.9586815 | C3orf58 | MWRLVPPKLGRLSRSLKLAALGSLLVLMVLHSPS | Core Human ORF Only |
| | 6452 | OIPI | | | LLASWQRNELTDRRFLQLNKCPACFGTSWCRRF | |
| | | _RF | | | LNGQVVFEAWGRLRLLDFLNVKNVYFAQYGEPR | |
| | | S | | | EGGRRRVVLKRLGSQRELAQLDQSICKRATGRP | |
| | | | | | RCDLLQAMPRTEFARLNGDVRLLTPEAVEGWSD | |
| | | | | | LVHCPSQRLLDRLVRRYAETKDSGSFLLRNLKDS | |
| | | | | | ERMQLLLTLAFNPEPLVLQSFPSDEGWPFAKYLG | |
| | | | | | ACGRMVAVNYVGEELWSYFNAPWEKRVDLAWQ | |
| | | | | | LMEIAEQLTNNDFEFALYLLDVSFDNFAVGPRDG | |
| | | | | | KVIIVDAENVLVADKRLIRQNKPENWDVWYESKF | |
| | | | | | DDCDKEACLSFSKEILCARATVDHNYYAVCQNLL | |
| | | | | | SRHATWRGTSGGLLHDPPSEIAKDGRLEALLDEC | |
| | | | | | ANPKKRYGRFQAAKELREYLAQLSNNVR | |
| 481 | JHU2 | NIV | 0.391406029 | SH3RF | MSPILGYWKIKGLVQPTRLLLEYLEEKYEEHLYER | Full Literal Sequence- |
| | 9408 | OIPI | | 2 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDDLTLLDLLECPVC | |
| | | | | | FEKLDVTAKVLPCQHTFCKPCLQRVFKAHKELRC | |
| | | | | | PECRTPVFSNIEALPANLLLVRLLDGVRSGQSSG | |
| | | | | | RGGSFRRPGTMTLQDGRKSRTNPRRLQASPFRL | |
| | | | | | VPNVRIHMDGVPRAKALCNYRGQNPGDLRFNKG | |
| | | | | | DIILLRRQLDENWYQGEINGISGNFPASSVEVIKQL | |
| | | | | | PQPPPLCRALYNFDLRGKDKSENQDCLTFLKDDII | |
| | | | | | TVISRVDENWAEGKLGDKVGIFPILFVEPNLTARH | |
| | | | | | LLEKNKGRQSSCTKNLSLVSSSSRGNTSTLRRGP | |
| | | | | | GSRRKVPGQFSITTALNTLNRMVHSPSGRHMVEI | |
| | | | | | STPVLISSSNPSVITQPMEKADVPSSCVGQVSTY | |
| | | | | | HPAPVSPGHSTAVVSLPGSQQHLSANMFVALHS | |
| | | | | | YSAHGPDELDLQKGEGVRVLGKCQDGWLRGVS | |
| | | | | | LVTGRVGIFPNNYVIPIFRKTSSFPDSRSPGLYTT | |
| | | | | | WTLSTSSVSSQGSISEGDPRQSRPFKSVFVPTAI | |
| | | | | | VNPVRSTAGPGTLGQGSLRKGRSSMRKNGSLQ | |
| | | | | | RPLQSGIPTLVVGSLRRSPTMVLRPQQFQFYQPQ | |
| | | | | | GIPSSPSAVVVEMGSKPALTGEPALTCISRGSEA | |
| | | | | | RIHSAASSLIMEDKEIPIKSEPLPKPPASAPPSILVK | |
| | | | | | PENSRNGIEKQVKTVRFQNYSPPPTKHYTSHPTS | |
| | | | | | GKPEQPATLKASQPEAASLGPEMTVLFAHRSGC | |
| | | | | | HSGQQTDLRRKSALAKATTLVSTASGTQTVFPSK | |
| 482 | JHU2 | NIV | 0.391406029 | SH3RF | MDDLTLLDLLECPVCFEKLDVTAKVLPCQHTFCK | Core Human ORF Only |
| | 9408 | OIPI | | 2 | PCLQRVFKAHKELRCPECRTPVFSNIEALPANLLL | |
| | | _RF | | | VRLLDGVRSGQSSGRGGSFRRPGTMTLQDGRK | |
| | | S | | | SRTNPRRLQASPFRLVPNVRIHMDGVPRAKALCN | |
| | | | | | YRGQNPGDLRFNKGDIILLRRQLDENWYQGEING | |
| | | | | | ISGNFPASSVEVIKQLPQPPPLCRALYNFDLRGKD | |
| | | | | | KSENQDCLTFLKDDIITVISRVDENWAEGKLGDKV | |
| | | | | | GIFPILFVEPNLTARHLLEKNKGRQSSCTKNLSLV | |
| | | | | | SSSSRGNTSTLRRGPGSRRKVPGQFSITTALNTL | |
| | | | | | NRMVHSPSGRHMVEISTPVLISSSNPSVITQPME | |
| | | | | | KADVPSSCVGQVSTYHPAPVSPGHSTAVVSLPG | |
| | | | | | SQQHLSANMFVALHSYSAHGPDELDLQKGEGVR | |
| | | | | | VLGKCQDGWLRGVSLVTGRVGIFPNNYVIPIFRKT | |
| | | | | | SSFPDSRSPGLYTTWTLSTSSVSSQGSISEGDPR | |
| | | | | | QSRPFKSVFVPTAIVNPVRSTAGPGTLGQGSLRK | |
| | | | | | GRSSMRKNGSLQRPLQSGIPTLVVGSLRRSPTM | |
| | | | | | VLRPQQFQFYQPQGIPSSPSAVVVEMGSKPALT | |
| | | | | | GEPALTCISRGSEARIHSAASSLIMEDKEIPIKSEP | |
| | | | | | LPKPPASAPPSILVKPENSRNGIEKQVKTVRFQNY | |
| | | | | | SPPPTKHYTSHPTSGKPEQPATLKASQPEAASLG | |
| | | | | | PEMTVLFAHRSGCHSGQQTDLRRKSALAKATTLV | |
| | | | | | STASGTQTVFPSK | |
| 483 | JHU2 | NIV | 0.245707506 | C15orf5 | MSPILGYWKIKGLVQPTRLLLEYLEEKYEEHLYER | Full Literal Sequence- |
| | 9511 | OIPI | | 3 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMELQGAQEDLGISLS | |
| | | | | | SPRRNHETRPGSKAKGRSSICLQASVWMAGGKL | |
| | | | | | RLRASEHLTQGHQQELRDWNLGEDASLLFSKSP | |
| | | | | | FGAGKLIQAPAHVFRQCWVQGNAWISCITKFDSK | |
| | | | | | RSPEVASSPSYLTVPRRSPLPVFLRPSDRCVCGG | |
| | | | | | CYLGKSTRRRACQSLLSDPLGVTFPTQTRP | |
| 484 | JHU2 | NIV | 0.245707506 | C15orf5 | MELQGAQEDLGISLSSPRRNHETRPGSKAKGRS | Core Human ORF Only |
| | 9511 | OIPI | | 3 | SICLQASVWMAGGKLRLRASEHLTQGHQQELRD | |
| | | _RF | | | WNLGEDASLLFSKSPFGAGKLIQAPAHVFRQCW | |
| | | S | | | VQGNAWISCITKFDSKRSPEVASSPSYLTVPRRS | |
| | | | | | PLPVFLRPSDRCVCGGCYLGKSTRRRACQSLLS | |
| | | | | | DPLGVTFPTQTRP | |
| 485 | JHU2 | NIV | 0.869561347 | ZNF72 | MSPILGYWKIKGLVQPTRLLLEYLEEKYEEHLYER | Full Literal Sequence- |
| | 9910 | OIPI | | 6 | DEGDKWRNKKFELGLEFPNLPYYIDGDVKLTQSM | (including GST-tag, 6- |
| | | _RF | | | AIIRYIADKHNMLGGCPKERAEISMLEGAVLDIRYG | His leader, cloning |
| | | S | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGLLTFRDVAIEFSL | |
| | | | | | EEWQCLDTAQKNLYRNVMLENYRNLAFLGIAVSK | |
| | | | | | PDLIICLEKEKEPWNMKRDEMVDEPPGR | |
| 486 | JHU2 | NIV | 0.869561347 | ZNF72 | MGLLTFRDVAIEFSLEEWQCLDTAQKNLYRNVML | Core Human ORF Only |
| | 9910 | OIPI | | 6 | ENYRNLAFLGIAVSKPDLIICLEKEKEPWNMKRDE | |
| | | _RF | | | MVDEPPGR | |
| | | S | | | | |
| 487 | JHUO | NIV | 0.213449942 | RGR | MSPILGYWKIKGLVQPTRLLLEYLEEKYEEHLYER | Full Literal Sequence- |
| | 0273 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAETSALPTGFGELE | |
| | | | | | VLAVGMVLLVEDPGAADSLPPTGAELGSCGQWD | |
| | | | | | QPECPRCSHIQPSPCLPQALALRLGRLPGSRLPG | |
| | | | | | LCDSVGQHLQQCSHRMGALSPLLHPPKCCHRSH | |
| | | | | | HFEWR | |
| 488 | JHUO | NIV | 0.213449942 | RGR | MAETSALPTGFGELEVLAVGMVLLVEDPGAADSL | Core Human ORF Only |
| | 0273 | OIPI | | | PPTGAELGSCGQWDQPECPRCSHIQPSPCLPQA | |
| | | _TO | | | LALRLGRLPGSRLPGLCDSVGQHLQQCSHRMGA | |
| | | X | | | LSPLLHPPKCCHRSHHFEWR | |
| 489 | JHUO | NIV | 2.328325255 | SGTA | MSPILGYWKIKGLVQPTRLLLEYLEEKYEEHLYER | Full Literal Sequence- |
| | 2671 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDNKKRLAYAIIQFLH | |
| | | | | | DQLRHGGLSSDAQESLEVAIQCLETAFGVTVEDS | |
| | | | | | DLALPQTLPEIFEAAATGKEMPQDLRSPARTPPS | |
| | | | | | EEDSAEAERLKTEGNEQMKVENFEAAVHFYGKAI | |
| | | | | | ELNPANAVYFCNRAAAYSKLGNYAGAVQDCERAI | |
| | | | | | CIDPAYSKAYGRMGLALSSLNKHVEAVAYYKKAL | |
| | | | | | ELDPDNETYKSNLKIAELKLREAPSPTGGVGSFDI | |
| | | | | | AGLLNNPGFMSMASNLMNNPQIQQLMSGMISGG | |
| | | | | | NNPLGTPGTSPSQNDLASLIQAGQQFAQQMQQQ | |
| | | | | | NPELIEQLRSQIRSRTPSASNDDQQE | |
| 490 | JHUO | NIV | 2.328325255 | SGTA | MDNKKRLAYAIIQFLHDQLRHGGLSSDAQESLEV | Core Human ORF Only |
| | 2671 | OIPI | | | AIQCLETAFGVTVEDSDLALPQTLPEIFEAAATGK | |
| | | _TO | | | EMPQDLRSPARTPPSEEDSAEAERLKTEGNEQM | |
| | | X | | | KVENFEAAVHFYGKAIELNPANAVYFCNRAAAYS | |
| | | | | | KLGNYAGAVQDCERAICIDPAYSKAYGRMGLALS | |
| | | | | | SLNKHVEAVAYYKKALELDPDNETYKSNLKIAELK | |
| | | | | | LREAPSPTGGVGSFDIAGLLNNPGFMSMASNLM | |
| | | | | | NNPQIQQLMSGMISGGNNPLGTPGTSPSQNDLA | |
| | | | | | SLIQAGQQFAQQMQQQNPELIEQLRSQIRSRTPS | |
| | | | | | ASNDDQQE | |
| 491 | JHUO | NIV | 1.0529735 | NCAPG | MSPILGYWKIKGLVQPTRLLLEYLEEKYEEHLYER | Full Literal Sequence- |
| | 3521 | OIPI | 7 | _frag | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNGIIESLILPGIISIHP | |
| | | | | | VVRNLAVLCLGCCGLQNQDFARKHFVLLLQVLQI | |
| | | | | | DDVTIKISALKAIFDQLMTFGIEPFKTKKIKTLHCEG | |
| | | | | | TEINSDDEQESKEVEETATAKNVLKLLSDFLDSEV | |
| | | | | | SELRTGAAEGLAKLMFSGLLVSSRILSRLILLWYN | |
| | | | | | PVTEEDVQLRHCLGVFFPVFAYASRTNQECFEEA | |
| | | | | | FLPTLQTLANAPASSPLAEIDITNVAELLVDLTRPS | |
| | | | | | GLNPQAKTSQDYQALTVHDNLAMKICNEILTSPC | |
| | | | | | SPEIRVYTKALSSLELSSHLAKDLLVLLNEILEQVK | |
| | | | | | DRTCLRALEKIKIQLEKGNKEFGDQAEAAQDATLT | |
| | | | | | TTTFQNEDEKNKEVYMTPLRGVKATQASKSTQLK | |
| | | | | | TNRGQRKVTVSARTNRRCQTAEADSESDHEVPE | |
| | | | | | PESEMKMRLPRRAKTAALEKSKLNLAQFLNEDLS | |
| 492 | JHUO | NIV | 1.0529735 | NCAPG | MNGIIESLILPGIISIHPVVRNLAVLCLGCCGLQNQ | Core Human ORF Only |
| | 3521 | OIPI | 7 | _frag | DFARKHFVLLLQVLQIDDVTIKISALKAIFDQLMTF | |
| | | _TO | | | GIEPFKTKKIKTLHCEGTEINSDDEQESKEVEETA | |
| | | X | | | TAKNVLKLLSDFLDSEVSELRTGAAEGLAKLMFS | |
| | | | | | GLLVSSRILSRLILLWYNPVTEEDVQLRHCLGVFF | |
| | | | | | PVFAYASRTNQECFEEAFLPTLQTLANAPASSPL | |
| | | | | | AEIDITNVAELLVDLTRPSGLNPQAKTSQDYQALT | |
| | | | | | VHDNLAMKICNEILTSPCSPEIRVYTKALSSLELSS | |
| | | | | | HLAKDLLVLLNEILEQVKDRTCLRALEKIKIQLEKG | |
| | | | | | NKEFGDQAEAAQDATLTTTTFQNEDEKNKEVYM | |
| | | | | | TPLRGVKATQASKSTQLKTNRGQRKVTVSARTN | |
| | | | | | RRCQTAEADSESDHEVPEPESEMKMRLPRRAKT | |
| | | | | | AALEKSKLNLAQFLNEDLS | |
| 493 | JHUO | NIV | 1.88462434 | C10orf5 | MSPILGYWKIKGLVQPTRLLLEYLEEKYEEHLYER | Full Literal Sequence- |
| | 3655 | OIPI | 6 | 3 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPKNAVVILRYGPYS | |
| | | | | | AAGLPVEHHTFRLQGLQAVLAIDGHEVILEKIEDW | |
| | | | | | NVVELMVNEEVIFHCNIKDLEFGKLTPSSDKRTTS | |
| | | | | | SSRLTFHQLSSPCGMKVSPLQQFPQKTQDLTCIV | |
| | | | | | LAQIGSCIHFQTNLCDLGWPGLDHMLISGLEKRG | |
| | | | | | TQPY | |
| 494 | JHUO | NIV | 1.88462434 | C10orf5 | MPKNAVVILRYGPYSAAGLPVEHHTFRLQGLQAV | Core Human ORF Only |
| | 3655 | OIPI | 6 | 3 | LAIDGHEVILEKIEDWNVVELMVNEEVIFHCNIKDL | |
| | | _TO | | | EFGKLTPSSDKRTTSSSRLTFHQLSSPCGMKVSP | |
| | | X | | | LQQFPQKTQDLTCIVLAQIGSCIHFQTNLCDLGWP | |
| | | | | | GLDHMLISGLEKRGTQPY | |
| 495 | JHUO | NIV | 0.01391479 | PDP2 | MSPILGYWKIKGLVQPTRLLLEYLEEKYEEHLYER | Full Literal Sequence- |
| | 4193 | OIPI | 2 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSSTVSYWILNSTRN | |
| | | | | | SIATLQGGRRLYSRYVSNRNKLKWRLFSRVPPTL | |
| | | | | | NSSPCGGFTLCKAYRHTSTEEDDFHLQLSPEQIN | |
| | | | | | EVLRAGETTHKILDLESRVPNSVLRFESNQLAANS | |
| | | | | | PVEDRRGVASCLQTNGLMFGIFDGHGGHACAQA | |
| | | | | | VSERLFYYVAVSLMSHQTLEHMEGAMESMKPLL | |
| | | | | | PILHWLKHPGDSIYKDVTSVHLDHLRVYWQELLD | |
| | | | | | LHMEMGLSIEEALMYSFQRLDSDISLEIQAPLEDE | |
| | | | | | VTRNLSLQVAFSGATACMAHVDGIHLHVANAGDC | |
| | | | | | RAILGVQEDNGMWSCLPLTRDHNAWNQAELSRL | |
| | | | | | KREHPESEDRTIIMEDRLLGVLIPCRAFGDVQLKW | |
| | | | | | SKELQRSILERGFNTEALNIYQFTPPHYYTPPYLT | |
| | | | | | AEPEVTYHRLRPQDKFLVLASDGLWDMLSNEDV | |
| | | | | | VRLVVGHLAEADWHKTDLAQRPANLGLMQSLLL | |
| | | | | | QRKASGLHEADQNAATRLIRHAIGNNEYGEMEAE | |
| | | | | | RLAAMLTLPEDLARMYRDDITVTVVYFNSESIGAY | |
| | | | | | YKGG | |
| 496 | JHUO | NIV | 0.01391479 | PDP2 | MSSTVSYWILNSTRNSIATLQGGRRLYSRYVSNR | Core Human ORF Only |
| | 4193 | OIPI | 2 | | NKLKWRLFSRVPPTLNSSPCGGFTLCKAYRHTST | |
| | | _TO | | | EEDDFHLQLSPEQINEVLRAGETTHKILDLESRVP | |
| | | X | | | NSVLRFESNQLAANSPVEDRRGVASCLQTNGLM | |
| | | | | | FGIFDGHGGHACAQAVSERLFYYVAVSLMSHQTL | |
| | | | | | EHMEGAMESMKPLLPILHWLKHPGDSIYKDVTSV | |
| | | | | | HLDHLRVYWQELLDLHMEMGLSIEEALMYSFQRL | |
| | | | | | DSDISLEIQAPLEDEVTRNLSLQVAFSGATACMAH | |
| | | | | | VDGIHLHVANAGDCRAILGVQEDNGMWSCLPLT | |
| | | | | | RDHNAWNQAELSRLKREHPESEDRTIIMEDRLLG | |
| | | | | | VLIPCRAFGDVQLKWSKELQRSILERGFNTEALNI | |
| | | | | | YQFTPPHYYTPPYLTAEPEVTYHRLRPQDKFLVL | |
| | | | | | ASDGLWDMLSNEDVVRLVVGHLAEADWHKTDLA | |
| | | | | | QRPANLGLMQSLLLQRKASGLHEADQNAATRLIR | |
| | | | | | HAIGNNEYGEMEAERLAAMLTLPEDLARMYRDDI | |
| | | | | | TVTVVYFNSESIGAYYKGG | |
| 497 | JHUO | NIV | 3.95002209 | SERPI | MSPILGYWKIKGLVQPTRLLLEYLEEKYEEHLYER | Full Literal Sequence- |
| | 4205 | OIPI | 4 | NF2 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVLLWGLLVLSWSC | |
| | | | | | LQGPCSVFSPVSAMEPLGRQLTSGPNQEQVSPL | |
| | | | | | TLLKLGNQEPGGQTALKSPPGVCSRDPTPEQTH | |
| | | | | | RLARAMMAFTADLFSLVAQTSTCPNLILSPLSVAL | |
| | | | | | ALSHLALGAQNHTLQRLQQVLHAGSGPCLPHLLS | |
| | | | | | RLCQDLGPGAFRLAARMYLQKGFPIKEDFLEQSE | |
| | | | | | QLFGAKPVSLTGKQEDDLANINQVWKEATEGKIQ | |
| | | | | | EFLSGLPEDTVLLLLNAIHFQGFWRNKFDPSLTQR | |
| | | | | | DSFHLDEQFTVPVEMMQARTYPLRWFLLEQPEIQ | |
| | | | | | VAHFPFKNNMSFVVLVPTHFEWNVSQVLANLSW | |
| | | | | | DTLHPPLVWERPTKVRLPKLYLKHQMDLVATLSQ | |
| | | | | | LGLQELFQAPDLRGISEQSLVVSGVQHQSTLELS | |
| | | | | | EVGVEAAAATSIAMSRMSLSSFSVNRPFLFFIFED | |
| | | | | | TTGLPLFVGSVRNPNPSAPRELKEQQDSPGNKD | |
| | | | | | FLQSLKGFPRGDKLFGPDLKLVPPMEEDYPQFGS | |
| | | | | | PK | |
| 498 | JHUO | NIV | 3.95002209 | SERPI | MVLLWGLLVLSWSCLQGPCSVFSPVSAMEPLGR | Core Human ORF Only |
| | 4205 | OIPI | 4 | NF2 | QLTSGPNQEQVSPLTLLKLGNQEPGGQTALKSPP | |
| | | _TO | | | GVCSRDPTPEQTHRLARAMMAFTADLFSLVAQT | |
| | | X | | | STCPNLILSPLSVALALSHLALGAQNHTLQRLQQV | |
| | | | | | LHAGSGPCLPHLLSRLCQDLGPGAFRLAARMYL | |
| | | | | | QKGFPIKEDFLEQSEQLFGAKPVSLTGKQEDDLA | |
| | | | | | NINQVWKEATEGKIQEFLSGLPEDTVLLLLNAIHF | |
| | | | | | QGFWRNKFDPSLTQRDSFHLDEQFTVPVEMMQ | |
| | | | | | ARTYPLRWFLLEQPEIQVAHFPFKNNMSFVVLVP | |
| | | | | | THFEWNVSQVLANLSWDTLHPPLVWERPTKVRL | |
| | | | | | PKLYLKHQMDLVATLSQLGLQELFQAPDLRGISE | |
| | | | | | QSLVVSGVQHQSTLELSEVGVEAAAATSIAMSRM | |
| | | | | | SLSSFSVNRPFLFFIFEDTTGLPLFVGSVRNPNPS | |
| | | | | | APRELKEQQDSPGNKDFLQSLKGFPRGDKLFGP | |
| | | | | | DLKLVPPMEEDYPQFGSPK | |
| 499 | JHUO | NIV | 1.22784833 | OSGIN | MSPILGYWKIKGLVQPTRLLLEYLEEKYEEHLYER | Full Literal Sequence- |
| | 4371 | OIPI | 3 | 1 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSSSRKDHLGASSS | |
| | | | | | EPLPVIIVGNGPSGICLSYLLSGYTPYTKPDAIHPH | |
| | | | | | PLLQRKLTEAPGVSILDQDLDYLSEGLEGRSQSP | |
| | | | | | VALLFDALLRPDTDFGGNMKSVLTWKHRKEHAIP | |
| | | | | | HVVLGRNLPGGAWHSIEGSMVILSQGQWMGLPD | |
| | | | | | LEVKDWMQKKRRGLRNSRATAGDIAHYYRDYVV | |
| | | | | | KKGLGHNFVSGAVVTAVEWGTPDPSSCGAQDSS | |
| | | | | | PLFQVSGFLTRNQAQQPFSLWARNVVLATGTFD | |
| | | | | | SPARLGIPGEALPFIHHELSALEAATRVGAVTPAS | |
| | | | | | DPVLIIGAGLSAADAVLYARHYNIPVIHAFRRAVDD | |
| | | | | | PGLVFNQLPKMLYPEYHKVHQMMREQSILSPSPY | |
| | | | | | EGYRSLPRHQLLCFKEDCQAVFQDLEGVEKVFG | |
| | | | | | VSLVLVLIGSHPDLSFLPGAGADFAVDPDQPLSAK | |
| | | | | | RNPIDVDPFTYQSTRQEGLYAMGPLAGDNFVRFV | |
| | | | | | QGGALAVASSLLRKETRKPP | |
| 500 | JHUO | NIV | 1.22784833 | OSGIN | MSSSRKDHLGASSSEPLPVIIVGNGPSGICLSYLL | Core Human ORF Only |
| | 4371 | OIPI | 3 | 1 | SGYTPYTKPDAIHPHPLLQRKLTEAPGVSILDQDL | |
| | | _TO | | | DYLSEGLEGRSQSPVALLFDALLRPDTDFGGNMK | |
| | | X | | | SVLTWKHRKEHAIPHVVLGRNLPGGAWHSIEGS | |
| | | | | | MVILSQGQWMGLPDLEVKDWMQKKRRGLRNSR | |
| | | | | | ATAGDIAHYYRDYVVKKGLGHNFVSGAVVTAVE | |
| | | | | | WGTPDPSSCGAQDSSPLFQVSGFLTRNQAQQPF | |
| | | | | | SLWARNVVLATGTFDSPARLGIPGEALPFIHHELS | |
| | | | | | ALEAATRVGAVTPASDPVLIIGAGLSAADAVLYAR | |
| | | | | | HYNIPVIHAFRRAVDDPGLVFNQLPKMLYPEYHK | |
| | | | | | VHQMMREQSILSPSPYEGYRSLPRHQLLCFKED | |
| | | | | | CQAVFQDLEGVEKVFGVSLVLVLIGSHPDLSFLP | |
| | | | | | GAGADFAVDPDQPLSAKRNPIDVDPFTYQSTRQE | |
| | | | | | GLYAMGPLAGDNFVRFVQGGALAVASSLLRKET | |
| | | | | | RKPP | |
| 501 | JHUO | NIV | 1.214620754 | SYT11 | MSPILGYWKIKGLVQPTRLLLEYLEEKYEEHLYER | Full Literal Sequence- |
| | 4782 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAEITNIRPSFDVSPV | |
| | | | | | VAGLIGASVLVVCVSVTVFVWSCCHQQAEKKHK | |
| | | | | | NPPYKFIHMLKGISIYPETLSNKKKIIKVRRDKDGP | |
| | | | | | GREGGRRNLLVDAAEAGLLSRDKDPRGPSSGSC | |
| | | | | | IDQLPIKMDYGEELRSPITSLTPGESKTTSPSSPEE | |
| | | | | | DVMLGSLTFSVDYNFPKKALVVTIQEAHGLPVMD | |
| | | | | | DQTQGSDPYIKMTILPDKRHRVKTRVLRKTLDPVF | |
| | | | | | DETFTFYGIPYSQLQDLVLHFLVLSFDRFSRDDVI | |
| | | | | | GEVMVPLAGVDPSTGKVQLTRDIIKRNIQKCISRG | |
| | | | | | ELQVSLSYQPVAQRMTVVVLKARHLPKMDITGLS | |
| | | | | | GNPYVKVNVYYGRKRIAKKKTHVKKCTLNPIFNES | |
| | | | | | FIYDIPTDLLPDISIEFLVIDFDRTTKNEVVGRLI | |
| | | | | | LGAHSVTASGAEHWREVCESPRKPVAKWHSLSEY | |
| 502 | JHUO | NIV | 1.214620754 | SYT11 | MAEITNIRPSFDVSPVVAGLIGASVLVVCVSVTVF | Core Human ORF Only |
| | 4782 | OIPI | | | VWSCCHQQAEKKHKNPPYKFIHMLKGISIYPETLS | |
| | | _TO | | | NKKKIIKVRRDKDGPGREGGRRNLLVDAAEAGLL | |
| | | X | | | SRDKDPRGPSSGSCIDQLPIKMDYGEELRSPITSL | |
| | | | | | TPGESKTTSPSSPEEDVMLGSLTFSVDYNFPKKA | |
| | | | | | LVVTIQEAHGLPVMDDQTQGSDPYIKMTILPDKRH | |
| | | | | | RVKTRVLRKTLDPVFDETFTFYGIPYSQLQDLVLH | |
| | | | | | FLVLSFDRFSRDDVIGEVMVPLAGVDPSTGKVQL | |
| | | | | | TRDIIKRNIQKCISRGELQVSLSYQPVAQRMTVVV | |
| | | | | | LKARHLPKMDITGLSGNPYVKVNVYYGRKRIAKK | |
| | | | | | KTHVKKCTLNPIFNESFIYDIPTDLLPDISIEFLV | |
| | | | | | IDFDRTTKNEVVGRLILGAHSVTASGAEHWREVCES | |
| | | | | | PRKPVAKWHSLSEY | |
| 503 | JHUO | NIV | 4.654470677 | ARL14 | MSPILGYWKIKGLVQPTRLLLEYLEEKYEEHLYER | Full Literal Sequence- |
| | 5769 | OIPI | | EP | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMMDPCSVGVQLRTT | |
| | | | | | NECHKTYYTRHTGFKTLQELSSNDMLLLQLRTGM | |
| | | | | | TLSGNNTICFHHVKIYIDRFEDLQKSCCDPFNIHKK | |
| | | | | | LAKKNLHVIDLDDATFLSAKFGRQLVPGWKLCPK | |
| | | | | | CTQIINGSVDVDTEDRQKRKPESDGRTAKALRSL | |
| | | | | | QFTNPGRQTEFAPETGKREKRRLTKNATAGSDR | |
| | | | | | QVIPAKSKVYDSQGLLIFSGMDLCDCLDEDCLGC | |
| | | | | | FYACPACGSTKCGAECRCDRKWLYEQIEIEGGEII | |
| | | | | | HNKHAG | |
| 504 | JHUO | NIV | 4.654470677 | ARL14 | MMDPCSVGVQLRTTNECHKTYYTRHTGFKTLQE | Core Human ORF Only |
| | 5769 | OIPI | | EP | LSSNDMLLLQLRTGMTLSGNNTICFHHVKIYIDRF | |
| | | _TO | | | EDLQKSCCDPFNIHKKLAKKNLHVIDLDDATFLSA | |
| | | X | | | KFGRQLVPGWKLCPKCTQIINGSVDVDTEDRQKR | |
| | | | | | KPESDGRTAKALRSLQFTNPGRQTEFAPETGKR | |
| | | | | | EKRRLTKNATAGSDRQVIPAKSKVYDSQGLLIFSG | |
| | | | | | MDLCDCLDEDCLGCFYACPACGSTKCGAECRCD | |
| | | | | | RKWLYEQIEIEGGEIIHNKHAG | |
| 505 | JHUO | NIV | 1.942954052 | GBA | MSPILGYWKIKGLVQPTRLLLEYLEEKYEEHLYER | Full Literal Sequence- |
| | 6259 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEFSSPSREECPKPL | |
| | | | | | SRVSIMAGSLTGLLLLQAVSWASGARPCIPKSFG | |
| | | | | | YSSVVCVCNATYCDSFDPPTFPALGTFSRYESTR | |
| | | | | | SGRRMELSMGPIQANHTGTGLLLTLQPEQKFQKV | |
| | | | | | KGFGGAMTDAAALNILALSPPAQNLLLKSYFSEE | |
| | | | | | GIGYNIIRVPMASCDFSIRTYTYADTPDDFQLHNF | |
| | | | | | SLPEEDTKLKIPLIHRALQLAQRPVSLLASPWTSP | |
| | | | | | TWLKTNGAVNGKGSLKGQPGDIYHQTWARYFVK | |
| | | | | | FLDAYAEHKLQFWAVTAENEPSAGLLSGYPFQCL | |
| | | | | | GFTPEHQRDFIARDLGPTLANSTHHNVRLLMLDD | |
| | | | | | QRLLLPHWAKVVLTDPEAAKYVHGIAVHWYLDFL | |
| | | | | | APAKATLGETHRLFPNTMLFASEACVGSKFWEQ | |
| | | | | | SVRLGSWDRGMQYSHSIITNLLYHVVGWTDWNL | |
| | | | | | ALNPEGGPNVWRNFVDSPIIVDITKDTFYKQPMFY | |
| | | | | | HLGHFSKFIPEGSQRVGLVASQKNDLDAVALMHP | |
| | | | | | DGSAVVVVLNRSSKDVPLTIKDPAVGFLETISPGY | |
| | | | | | SIHTYLWRRQ | |
| 506 | JHUO | NIV | 1.942954052 | GBA | MEFSSPSREECPKPLSRVSIMAGSLTGLLLLQAV | Core Human ORF Only |
| | 6259 | OIPI | | | SWASGARPCIPKSFGYSSVVCVCNATYCDSFDP | |
| | | _TO | | | PTFPALGTFSRYESTRSGRRMELSMGPIQANHTG | |
| | | X | | | TGLLLTLQPEQKFQKVKGFGGAMTDAAALNILALS | |
| | | | | | PPAQNLLLKSYFSEEGIGYNIIRVPMASCDFSIRTY | |
| | | | | | TYADTPDDFQLHNFSLPEEDTKLKIPLIHRALQLA | |
| | | | | | QRPVSLLASPWTSPTWLKTNGAVNGKGSLKGQP | |
| | | | | | GDIYHQTWARYFVKFLDAYAEHKLQFWAVTAEN | |
| | | | | | EPSAGLLSGYPFQCLGFTPEHQRDFIARDLGPTL | |
| | | | | | ANSTHHNVRLLMLDDQRLLLPHWAKVVLTDPEAA | |
| | | | | | KYVHGIAVHWYLDFLAPAKATLGETHRLFPNTML | |
| | | | | | FASEACVGSKFWEQSVRLGSWDRGMQYSHSIIT | |
| | | | | | NLLYHVVGWTDWNLALNPEGGPNVWRNFVDSPI | |
| | | | | | IVDITKDTFYKQPMFYHLGHFSKFIPEGSQRVGLV | |
| | | | | | ASQKNDLDAVALMHPDGSAVVVVLNRSSKDVPL | |
| | | | | | TIKDPAVGFLETISPGYSIHTYLWRRQ | |
| 507 | JHUO | NIV | 2.225033684 | DHX29 | MSPILGYWKIKGLVQPTRLLLEYLEEKYEEHLYER | Full Literal Sequence- |
| | 7426 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGGKNKKHKAPAAA | |
| | | | | | VVRAAVSASRAKSAEAGIAGEAQSKKPVSRPATA | |
| | | | | | AAAAAGSREPRVKQGPKIYSFNSTNDSSGPANLD | |
| | | | | | KSILKVVINNKLEQRIIGVINEHKKQNNDKGMISGR | |
| | | | | | LTAKKLQDLYMALQAFSFKTKDIEDAMTNTLLYGG | |
| | | | | | DLHSALDWLCLNLSDDALPEGFSQEFEEQQPKS | |
| | | | | | RPKFQSPQIQATISPPLQPKTKTYEEDPKSKPKKE | |
| | | | | | EKNMEVNMKEWILRYAEQQNEEEKNENSKSLEE | |
| | | | | | EEKFDPNERYLHLAAKLLDAKEQAATFKLEKNKQ | |
| | | | | | GQKEAQEKIRKFQREMETLEDHPVFNPAMKISHQ | |
| | | | | | QNERKKPPVATEGESALNFNLFEKSAAATEEEKD | |
| | | | | | KKKEPHDVRNFDYTARSWTGKSPKQFLIDVWRK | |
| | | | | | NLPKSPNPSFEKVPVGRYWKCRVRVIKSEDDVLV | |
| | | | | | VCPTILTEDGMQAQHLGATLALYRLVKGQSVHQL | |
| | | | | | LPPTYRDVWLEWSDAEKKREELNKMETNKPRDL | |
| | | | | | FIAKLLNKLKQQQQQQQQHSENKRENSEDPEES | |
| | | | | | WENLVSDEDFSALSLESANVEDLEPVRNLFRKLQ | |
| | | | | | STPKYQKLLKERQQLPVFKHRDSIVETLKRHRVV | |
| | | | | | VVAGETGSGKSTQVPHFLLEDLLLNEWEASKCNI | |
| | | | | | VCTQHRRISAVSLANRVCDELGCENGPGGRNSL | |
| | | | | | CGYQIRMESRACESTRLLYCTTGVLLRKLQEDGL | |
| | | | | | LSNVSHVIVDEVHERSVQSDFLLIILKEILQKRSDL | |
| | | | | | HLILMSATVDSEKFSTYFTHCPILRISGRSYPVEVF | |
| | | | | | HLEDIIEETGFVLEKDSEYCQKFLEEEEEVTINVTS | |
| | | | | | KAGGIKKYQEYIPVQTGAHADLNPFYQKYSSRTQ | |
| | | | | | HAILYMNPHKINLDLILELLAYLDKSPQFRNIEGAV | |
| | | | | | LIFLPGLAHIQQLYDLLSNDRRFYSERYKVIALHSIL | |
| | | | | | STQDQAAAFTLPPPGVRKIVLATNIAETGITIPDVV | |
| | | | | | FVIDTGRTKENKYHESSQMSSLVETFVSKASALQ | |
| | | | | | RQGRAGRVRDGFCFRMYTRERFEGFMDYSVPEI | |
| | | | | | LRVPLEELCLHIMKCNLGSPEDFLSKALDPPQLQV | |
| | | | | | ISNAMNLLRKIGACELNEPKLTPLGQHLAALPVNV | |
| | | | | | KIGKMLIFGAIFGCLDPVATLAAVMTEKSPFTTPIG | |
| | | | | | RKDEADLAKSALAMADSDHLTIYNAYLGWKKARQ | |
| | | | | | EGGYRSEITYCRRNFLNRTSLLTLEDVKQELIKLV | |
| | | | | | KAAGFSSSTTSTSWEGNRASQTLSFQEIALLKAV | |
| | | | | | LVAGLYDNVGKIIYTKSVDVTEKLACIVETAQGKA | |
| | | | | | QVHPSSVNRDLQTHGWLLYQEKIRYARVYLRETT | |
| | | | | | LITPFPVLLFGGDIEVQHRERLLSIDGWIYFQAPVK | |
| | | | | | IAVIFKQLRVLIDSVLRKKLENPKMSLENDKILQII | |
| | | | | | TELIKTENN | |
| 508 | JHUO | NIV | 2.225033684 | DHX29 | MGGKNKKHKAPAAAVVRAAVSASRAKSAEAGIA | Core Human ORF Only |
| | 7426 | OIPI | | | GEAQSKKPVSRPATAAAAAAGSREPRVKQGPKIY | |
| | | _TO | | | SFNSTNDSSGPANLDKSILKVVINNKLEQRIIGVIN | |
| | | X | | | EHKKQNNDKGMISGRLTAKKLQDLYMALQAFSFK | |
| | | | | | TKDIEDAMTNTLLYGGDLHSALDWLCLNLSDDAL | |
| | | | | | PEGFSQEFEEQQPKSRPKFQSPQIQATISPPLQP | |
| | | | | | KTKTYEEDPKSKPKKEEKNMEVNMKEWILRYAE | |
| | | | | | QQNEEEKNENSKSLEEEEKFDPNERYLHLAAKLL | |
| | | | | | DAKEQAATFKLEKNKQGQKEAQEKIRKFQREME | |
| | | | | | TLEDHPVFNPAMKISHQQNERKKPPVATEGESAL | |
| | | | | | NFNLFEKSAAATEEEKDKKKEPHDVRNFDYTARS | |
| | | | | | WTGKSPKQFLIDWVRKNLPKSPNPSFEKVPVGR | |
| | | | | | YWKCRVRVIKSEDDVLVVCPTILTEDGMQAQHLG | |
| | | | | | ATLALYRLVKGQSVHQLLPPTYRDVWLEWSDAE | |
| | | | | | KKREELNKMETNKPRDLFIAKLLNKLKQQQQQQQ | |
| | | | | | QHSENKRENSEDPEESWENLVSDEDFSALSLES | |
| | | | | | ANVEDLEPVRNLFRKLQSTPKYQKLLKERQQLPV | |
| | | | | | FKHRDSIVETLKRHRVVVVAGETGSGKSTQVPHF | |
| | | | | | LLEDLLLNEWEASKCNIVCTQHRRISAVSLANRVC | |
| | | | | | DELGCENGPGGRNSLCGYQIRMESRACESTRLL | |
| | | | | | YCTTGVLLRKLQEDGLLSNVSHVIVDEVHERSVQ | |
| | | | | | SDFLLIILKEILQKRSDLHLILMSATVDSEKFSTYFT | |
| | | | | | HCPILRISGRSYPVEVFHLEDIIEETGFVLEKDSEY | |
| | | | | | CQKFLEEEEEVTINVTSKAGGIKKYQEYIPVQTGA | |
| | | | | | HADLNPFYQKYSSRTQHAILYMNPHKINLDLILELL | |
| | | | | | AYLDKSPQFRNIEGAVLIFLPGLAHIQQLYDLLSND | |
| | | | | | RRFYSERYKVIALHSILSTQDQAAAFTLPPPGVRKI | |
| | | | | | VLATNIAETGITIPDVVFVIDTGRTKENKYHESSQM | |
| | | | | | SSLVETFVSKASALQRQGRAGRVRDGFCFRMYT | |
| | | | | | RERFEGFMDYSVPEILRVPLEELCLHIMKCNLGSP | |
| | | | | | EDFLSKALDPPQLQVISNAMNLLRKIGACELNEPK | |
| | | | | | LTPLGQHLAALPVNVKIGKMLIFGAIFGCLDPVATL | |
| | | | | | AAVMTEKSPFTTPIGRKDEADLAKSALAMADSDH | |
| | | | | | LTIYNAYLGWKKARQEGGYRSEITYCRRNFLNRT | |
| | | | | | SLLTLEDVKQELIKLVKAAGFSSSTTSTSWEGNRA | |
| | | | | | SQTLSFQEIALLKAVLVAGLYDNVGKIIYTKSVDVT | |
| | | | | | EKLACIVETAQGKAQVHPSSVNRDLQTHGWLLY | |
| | | | | | QEKIRYARVYLRETTLITPFPVLLFGGDIEVQHRER | |
| | | | | | LLSIDGWIYFQAPVKIAVIFKQLRVLIDSVLRKKLEN | |
| | | | | | PKMSLENDKILQIITELIKTENN | |
| 509 | JHUO | NIV | 1.812297311 | CAPN7 | MSPILGYWKIKGLVQPTRLLLEYLEEKYEEHLYER | Full Literal Sequence- |
| | 7888 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDATALERDAVQFA | |
| | | | | | RLAVQRDHEGRYSEAVFYYKEAAQALIYAEMAGS | |
| | | | | | SLENIQEKITEYLERVQALHSAVQSKSADPLKSKH | |
| | | | | | QLDLERAHFLVTQAFDEDEKENVEDAIELYTEAVD | |
| | | | | | LCLKTSYETADKVLQNKLKQLARQALDRAEALSE | |
| | | | | | PLTKPVGKISSTSVKPKPPPVRAHFPLGANPFLER | |
| | | | | | PQSFISPQSCDAQGQRYTAEEIEVLRTTSKINGIE | |
| | | | | | YVPFMNVDLRERFAYPMPFCDRWGKLPLSPKQK | |
| | | | | | TTFSKVWRPEDLTNNPTMIYTVSSFSIKQTIVSDC | |
| | | | | | SFVASLAISAAYERRFNKKLITGIIYPQNKDGEPEY | |
| | | | | | NPCGKYMVKLHLNGVPRKVIIDDQLPVDHKGELL | |
| | | | | | CSYSNNKSELVWSLIEKAYMKVMGGYDFPGSNS | |
| | | | | | NIDLHALTGWIPERIAMHSDSQTFSKDNSFRMLY | |
| | | | | | QRFHKGDVLITASTGMMTEAEGEKWGLVPTHAY | |
| | | | | | AVLDIREFKGLRFIQLKNPWSHLRWKGRYSENDV | |
| | | | | | KNWTPELQKYLNFDPRTAQKIDNGIFWISWDDLC | |
| | | | | | QYYDVIYLSWNPGLFKESTCIHSTWDAKQGPVKD | |
| | | | | | AYSLANNPQYKLEVQCPQGGAAVWVLLSRHITDK | |
| | | | | | DDFANNREFITMVVYKTDGKKVYYPADPPPYIDGI | |
| | | | | | RINSPHYLTKIKLTTPGTHTFTLVVSQYEKQNTIHY | |
| | | | | | TVRVYSACSFTFSKIPSPYTLSKRINGKWSGQSA | |
| | | | | | GGCGNFQETHKNNPIYQFHIEKTGPLLIELRGPRQ | |
| | | | | | YSVGFEVVTVSTLGDPGPHGFLRKSSGDYRCGF | |
| | | | | | CYLELENIPSGIFNIIPSTFLPKQEGPFFLDFNSI | |
| | | | | | IPIKITQLQ | |
| 510 | JHUO | NIV | 1.812297311 | CAPN7 | MDATALERDAVQFARLAVQRDHEGRYSEAVFYY | Core Human ORF Only |
| | 7888 | OIPI | | | KEAAQALIYAEMAGSSLENIQEKITEYLERVQALH | |
| | | _TO | | | SAVQSKSADPLKSKHQLDLERAHFLVTQAFDEDE | |
| | | X | | | KENVEDAIELYTEAVDLCLKTSYETADKVLQNKLK | |
| | | | | | QLARQALDRAEALSEPLTKPVGKISSTSVKPKPPP | |
| | | | | | VRAHFPLGANPFLERPQSFISPQSCDAQGQRYTA | |
| | | | | | EEIEVLRTTSKINGIEYVPFMNVDLRERFAYPMPF | |
| | | | | | CDRWGKLPLSPKQKTTFSKVWRPEDLTNNPTMI | |
| | | | | | YTVSSFSIKQTIVSDCSFVASLAISAAYERRFNKKL | |
| | | | | | ITGIIYPQNKDGEPEYNPCGKYMVKLHLNGVPRK | |
| | | | | | VIIDDQLPVDHKGELLCSYSNNKSELWVSLIEKAY | |
| | | | | | MKVMGGYDFPGSNSNIDLHALTGWIPERIAMHSD | |
| | | | | | SQTFSKDNSFRMLYQRFHKGDVLITASTGMMTEA | |
| | | | | | EGEKWGLVPTHAYAVLDIREFKGLRFIQLKNPWS | |
| | | | | | HLRWKGRYSENDVKNWTPELQKYLNFDPRTAQK | |
| | | | | | IDNGIFWISWDDLCQYYDVIYLSWNPGLFKESTCI | |
| | | | | | HSTWDAKQGPVKDAYSLANNPQYKLEVQCPQG | |
| | | | | | GAAVVWLLSRHITDKDDFANNREFITMVVYKTDG | |
| | | | | | KKVYYPADPPPYIDGIRINSPHYLTKIKLTTPGTHT | |
| | | | | | FTLVVSQYEKQNTIHYTVRVYSACSFTFSKIPSPY | |
| | | | | | TLSKRINGKWSGQSAGGCGNFQETHKNNPIYQF | |
| | | | | | HIEKTGPLLIELRGPRQYSVGFEVVTVSTLGDPGP | |
| | | | | | HGFLRKSSGDYRCGFCYLELENIPSGIFNIIPSTFL | |
| | | | | | PKQEGPFFLDFNSIIPIKITQLQ | |
| 511 | JHUO | NIV | 2.350098405 | CCL17 | MSPILGYWKIKGLVQPTRLLLEYLEEKYEEHLYER | Full Literal Sequence- |
| | 7890 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAPLKMLALVTLLLG | |
| | | | | | ASLQHIHAARGTNVGRECCLEYFKGAIPLRKLKT | |
| | | | | | WYQTSEDCSRDAIVFVTVQGRAICSDPNNKRVKN | |
| | | | | | AVKYLQSLERS | |
| 512 | JHUO | NIV | 2.350098405 | CCL17 | MAPLKMLALVTLLLGASLQHIHAARGTNVGRECC | Core Human ORF Only |
| | 7890 | OI | | | LEYFKGAIPLRKLKTWYQTSEDCSRDAIVFVTVQG | |
| | | PI | | | RAICSDPNNKRVKNAVKYLQSLERS | |
| | | _TO | | | | |
| | | X | | | | |
| 513 | JHUO | NIV | 1.244388489 | GPR17 | MSPILGYWKIKGLVQPTRLLLEYLEEKYEEHLYER | Full Literal Sequence- |
| | 7912 | OIPI | | 6 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGHNGSWISPNASE | |
| | | | | | PHNASGAEAAGVNRSALGEFGEAQLYRQFTTTV | |
| | | | | | QVVIFIGSLLGNFMVLWSTCRTTVFKSVTNRFIKN | |
| | | | | | LACSGICASLVCVPFDIILSTSPHCCVWVIYTMLFC | |
| | | | | | KVVKFLHKVFCSVTILSFPAIALDRYYSVLYPLERK | |
| | | | | | ISDAKSRELVMYIWAHAVVASVPVFAVTNVADIYA | |
| | | | | | TSTCTEVWSNSLGHLVYVLVYNITTVIVPVVVVFL | |
| | | | | | FLILIRRALSASQKKKVIIAALRTPQNTISIPYASQR | |
| | | | | | EAELHATLLSMVMVFILCSVPYATLVVYQTVLNVP | |
| | | | | | DTSVFLLLTAVWLPKVSLLANPVLFLTVNKSVRKC | |
| | | | | | LIGTLVQLHHRYSRRNVVSTGSGMAEASLEPSIR | |
| | | | | | SGSQLLEMFHIGQQQIFKPTEDEEESEAKYIGSAD | |
| | | | | | FQAKEIFSTCLEGEQGPQFAPSAPPLSTVDSVSQ | |
| | | | | | VAPAAPVEPETFPDKYSLQFGFGPFELPPQWLSE | |
| | | | | | TRNSKKRLLPPLGNTPEELIQTKVPKVGRVERKM | |
| | | | | | SRNNKVSIFPKVDS | |
| 514 | JHUO | NIV | 1.244388489 | GPR17 | MGHNGSWISPNASEPHNASGAEAAGVNRSALGE | Core Human ORF Only |
| | 7912 | OIPI | | 6 | FGEAQLYRQFTTTVQVVIFIGSLLGNFMVLWSTC | |
| | | _TO | | | RTTVFKSVTNRFIKNLACSGICASLVCVPFDIILSTS | |
| | | X | | | PHCCVWVIYTMLFCKVVKFLHKVFCSVTILSFPAIA | |
| | | | | | LDRYYSVLYPLERKISDAKSRELVMYIWAHAVVAS | |
| | | | | | VPVFAVTNVADIYATSTCTEVWSNSLGHLVYVLVYN | |
| | | | | | ITTVIVPVVVVFLFLILIRRALSASQKKKVIIAALR | |
| | | | | | TPQNTISIPYASQREAELHATLLSMVMVFILCSVPY | |
| | | | | | ATLVVYQTVLNVPDTSVFLLLTAVWLPKVSLLANP | |
| | | | | | VLFLTVNKSVRKCLIGTLVQLHHRYSRRNVVSTG | |
| | | | | | SGMAEASLEPSIRSGSQLLEMFHIGQQQIFKPTED | |
| | | | | | EEESEAKYIGSADFQAKEIFSTCLEGEQGPQFAP | |
| | | | | | SAPPLSTVDSVSQVAPAAPVEPETFPDKYSLQFG | |
| | | | | | FGPFELPPQWLSETRNSKKRLLPPLGNTPEELIQT | |
| | | | | | KVPKVGRVERKMSRNNKVSIFPKVDS | |
| 515 | JHUO | NIV | 0.102130884 | TDRD3 | MSPILGYWKIKGLVQPTRLLLEYLEEKYEEHLYER | Full Literal Sequence- |
| | 8240 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLRLQMTDGHISCTA | |
| | | | | | VEFSYMSKISLNTPPGTKVKLSGIVDIKNGFLLLND | |
| | | | | | SNTTVLGGEVEHLIEKWELQRSLSKHNRSNIGTE | |
| | | | | | GGPPPFVPFGQKCVSHVQVDSRELDRRKTLQVT | |
| | | | | | MPVKPTNDNDEFEKQRTAAIAEVAKSKETKTFGG | |
| | | | | | GGGGARSNLNMNAAGNRNREVLQKEKSTKSEG | |
| | | | | | KHEGVYRELVDEKALKHITEMGFSKEASRQALMD | |
| | | | | | NGNNLEAALNVLLTSNKQKPVMGPPLRGRGKGR | |
| | | | | | GRIRSEDEEDLGNARPSAPSTLFDFLESKMGTLN | |
| | | | | | VEEPKSQPQQLHQGQYRSSNTEQNGVKDNNHL | |
| | | | | | RHPPRNDTRQPRNEKPPRFQRDSQNSKSVLEGS | |
| | | | | | GLPRNRGSERPSTSSVSEVWAEDRIKCDRPYSR | |
| | | | | | YDRTKDTSYPLGSQHSDGAFKKRDNSMQSRSGK | |
| | | | | | GPSFAEAKENPLPQGSVDYNNQKRGKRESQTSI | |
| | | | | | PDYFYDRKSQTINNEAFSGIKIEKHFNVNTDYQNP | |
| | | | | | VRSNSFIGVPNGEVEMPLKGRRIGPIKPAGPVTAV | |
| | | | | | PCDDKIFYNSGPKRRSGPIKPEKILESSIPMEYAK | |
| | | | | | MWKPGDECFALYWEDNKFYRAEVEALHSSGMT | |
| | | | | | AVVKFIDYGNYEEVLLSNIKPIQTEAWEEEGTYDQ | |
| | | | | | TLEFRRGGDGQPRRSTRPTQQFYQPPRARN | |
| 516 | JHUO | NIV | 0.102130884 | TDRD3 | MLRLQMTDGHISCTAVEFSYMSKISLNTPPGTKV | Core Human ORF Only |
| | 8240 | OIPI | | | KLSGIVDIKNGFLLLNDSNTTVLGGEVEHLIEKWEL | |
| | | _TO | | | QRSLSKHNRSNIGTEGGPPPFVPFGQKCVSHVQ | |
| | | X | | | VDSRELDRRKTLQVTMPVKPTNDNDEFEKQRTA | |
| | | | | | AIAEVAKSKETKTFGGGGGGARSNLNMNAAGNR | |
| | | | | | NREVLQKEKSTKSEGKHEGVYRELVDEKALKHIT | |
| | | | | | EMGFSKEASRQALMDNGNNLEAALNVLLTSNKQ | |
| | | | | | KPVMGPPLRGRGKGRGRIRSEDEEDLGNARPSA | |
| | | | | | PSTLFDFLESKMGTLNVEEPKSQPQQLHQGQYR | |
| | | | | | SSNTEQNGVKDNNHLRHPPRNDTRQPRNEKPPR | |
| | | | | | FQRDSQNSKSVLEGSGLPRNRGSERPSTSSVSE | |
| | | | | | VWAEDRIKCDRPYSRYDRTKDTSYPLGSQHSDG | |
| | | | | | AFKKRDNSMQSRSGKGPSFAEAKENPLPQGSVD | |
| | | | | | YNNQKRGKRESQTSIPDYFYDRKSQTINNEAFSG | |
| | | | | | IKIEKHFNVNTDYQNPVRSNSFIGVPNGEVEMPLK | |
| | | | | | GRRIGPIKPAGPVTAVPCDDKIFYNSGPKRRSGPI | |
| | | | | | KPEKILESSIPMEYAKMWKPGDECFALYWEDNKF | |
| | | | | | YRAEVEALHSSGMTAVVKFIDYGNYEEVLLSNIKP | |
| | | | | | IQTEAWEEEGTYDQTLEFRRGGDGQPRRSTRPT | |
| | | | | | QQFYQPPRARN | |
| 517 | JHUO | NIV | 0.882157329 | CDC23 | MSPILGYWKIKGLVQPTRLLLEYLEEKYEEHLYER | Full Literal Sequence- |
| | 8372 | OIPI | | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVPVAVTAAVAPVLS | |
| | | | | | INSDFSDLREIKKQLLLIAGLTRERGLLHSSKWSAE | |
| | | | | | LAFSLPALPLAELQPPPPITEEDAQDMDAYTLAKA | |
| | | | | | YFDVKEYDRAAHFLHGCNSKKAYFLYMYSRYLS | |
| | | | | | GEKKKDDETVDSLGPLEKGQVKNEALRELRVELS | |
| | | | | | KKHQARELDGFGLYLYGVVLRKLDLVKEAIDVFVE | |
| | | | | | ATHVLPLHWGAWLELCNLITDKEMLKFLSLPDTW | |
| | | | | | MKEFFLAHIYTELQLIEEALQKYQNLIDVGFSKSSY | |
| | | | | | IVSQIAVAYHNIRDIDKALSIFNELRKQDPYRIENM | |
| | | | | | DTFSNLLYVRSMKSELSYLAHNLCEIDKYRVETCC | |
| | | | | | VIGNYYSLRSQHEKAALYFQRALKLNPRYLGAWT | |
| | | | | | LMGHEYMEMKNTSAAIQAYRHAIEVNKRDYRAW | |
| | | | | | YGLGQTYEILKMPFYCLYYYRRAHQLRPNDSRML | |
| | | | | | VALGECYEKLNQLVEAKKCYWRAYAVGDVEKMA | |
| | | | | | LVKLAKLHEQLTESEQAAQCYIKYIQDIYSCGEIVE | |
| | | | | | HLEESTAFRYLAQYYFKCKLWDEASTCAQKCCAF | |
| | | | | | NDTREEGKALLRQILQLRNQGETPTTEVPAPFFLP | |
| | | | | | ASLSANNTPTRRVSPLNLSSVTP | |
| 518 | JHUO | NIV | 0.882157329 | CDC23 | MVPVAVTAAVAPVLSINSDFSDLREIKKQLLLIAGL | Core Human ORF Only |
| | 8372 | OIPI | | | TRERGLLHSSKWSAELAFSLPALPLAELQPPPPIT | |
| | | _TO | | | EEDAQDMDAYTLAKAYFDVKEYDRAAHFLHGCN | |
| | | X | | | SKKAYFLYMYSRYLSGEKKKDDETVDSLGPLEKG | |
| | | | | | QVKNEALRELRVELSKKHQARELDGFGLYLYGVV | |
| | | | | | LRKLDLVKEAIDVFVEATHVLPLHWGAWLELCNLI | |
| | | | | | TDKEMLKFLSLPDTWMKEFFLAHIYTELQLIEEAL | |
| | | | | | QKYQNLIDVGFSKSSYIVSQIAVAYHNIRDIDKALSI | |
| | | | | | FNELRKQDPYRIENMDTFSNLLYVRSMKSELSYL | |
| | | | | | AHNLCEIDKYRVETCCVIGNYYSLRSQHEKAALYF | |
| | | | | | QRALKLNPRYLGAWTLMGHEYMEMKNTSAAIQA | |
| | | | | | YRHAIEVNKRDYRAWYGLGQTYEILKMPFYCLYY | |
| | | | | | YRRAHQLRPNDSRMLVALGECYEKLNQLVEAKK | |
| | | | | | CYWRAYAVGDVEKMALVKLAKLHEQLTESEQAA | |
| | | | | | QCYIKYIQDIYSCGEIVEHLEESTAFRYLAQYYFKC | |
| | | | | | KLWDEASTCAQKCCAFNDTREEGKALLRQILQLR | |
| | | | | | NQGETPTTEVPAPFFLPASLSANNTPTRRVSPLN | |
| | | | | | LSSVTP | |
| 519 | JHUO | NIV | 1.954651163 | SCARA | MSPILGYWKIKGLVQPTRLLLEYLEEKYEEHLYER | Full Literal Sequence- |
| | 8907 | OIPI | | 5 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMENKAMYLHTVSDC | |
| | | | | | DTSSICEDSFDGRSLSKLNLCEDGPCHKRRASIC | |
| | | | | | CTQLGSLSALKHAVLGLYLLVFLILVGIFILAVSRPR | |
| | | | | | SSPDDLKALTRNVNRLNESFRDLQLRLLQAPLQA | |
| | | | | | DLTEQVWKVQDALQNQSDSLLALAGAVQRLEGA | |
| | | | | | LWGLQAQAVQTEQAVALLRDRTGQQSDTAQLEL | |
| | | | | | YQLQVESNSSQLLLRRHAGLLDGLARRVGILGEE | |
| | | | | | LADVGGVLRGLNHSLSYDVALHRTRLQDLRVLVS | |
| | | | | | NASEDTRRLRLAHVGMELQLKQELAMLNAVTEDL | |
| | | | | | RLKDWEHSIALRNISLAKGPPGPKGDQGDEGKE | |
| | | | | | GRPGIPGLPGLRGLPGERGTPGLPGPKGDDGKL | |
| | | | | | GATGPMGMRGFKGDRGPKGEKGEKGDRAGDA | |
| | | | | | SKDILLGPWDMVLAQG | |
| 520 | JHUO | NIV | 1.954651163 | SCARA | MENKAMYLHTVSDCDTSSICEDSFDGRSLSKLNL | Core Human ORF Only |
| | 8907 | OIPI | | 5 | CEDGPCHKRRASICCTQLGSLSALKHAVLGLYLL | |
| | | _TO | | | VFLILVGIFILAVSRPRSSPDDLKALTRNVNRLNES | |
| | | X | | | FRDLQLRLLQAPLQADLTEQVWKVQDALQNQSD | |
| | | | | | SLLALAGAVQRLEGALWGLQAQAVQTEQAVALL | |
| | | | | | RDRTGQQSDTAQLELYQLQVESNSSQLLLRRHA | |
| | | | | | GLLDGLARRVGILGEELADVGGVLRGLNHSLSYD | |
| | | | | | VALHRTRLQDLRVLVSNASEDTRRLRLAHVGMEL | |
| | | | | | QLKQELAMLNAVTEDLRLKDWEHSIALRNISLAKG | |
| | | | | | PPGPKGDQGDEGKEGRPGIPGLPGLRGLPGERG | |
| | | | | | TPGLPGPKGDDGKLGATGPMGMRGFKGDRGPK | |
| | | | | | GEKGEKGDRAGDASKDILLGPWDMVLAQG | |
| 521 | JHUO | NIV | 2.8499584 | TES | MSPILGYWKIKGLVQPTRLLLEYLEEKYEEHLYER | Full Literal Sequence- |
| | 9106 | OIPI | 9 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMDLENKVKKMGLGH | |
| | | | | | EQGFGAPCLKCKEKCEGFELHFWRKICRNCKCG | |
| | | | | | QEEHDVLLSNEEDRKVGKLFEDTKYTTLIAKLKSD | |
| | | | | | GIPMYKRNVMILTNPVAAKKNVSINTVTYEWAPPV | |
| | | | | | QNQALARQYMQMLPKEKQPVAGSEGAQYRKKQ | |
| | | | | | LAKQLPAHDQDPSKCHELSPREVKEMEQFVKKY | |
| | | | | | KSEALGVGDVKLPCEMDAQGPKQMNIPGGDRST | |
| | | | | | PAAVGAMEDKSAEHKRTQYSCYCCKLSMKEGDP | |
| | | | | | AIYAERAGYDKLWHPACFVCSTCHELLVDMIYFW | |
| | | | | | KNEKLYCGRHYCDSEKPRCAGCDELIFSNEYTQA | |
| | | | | | ENQNWHLKHFCCFDCDSILAGEIYVMVNDKPVCK | |
| | | | | | PCYVKNHAVVCQGCHNAIDPEVQRVTYNNFSWH | |
| | | | | | ASTECFLCSCCSKCLIGQKFMPVEGMVFCSVECK | |
| | | | | | KRMS | |
| 522 | JHUO | NIV | 2.8499584 | TES | MDLENKVKKMGLGHEQGFGAPCLKCKEKCEGFE | Core Human ORF Only |
| | 9106 | OIPI | 9 | | LHFWRKICRNCKCGQEEHDVLLSNEEDRKVGKL | |
| | | _TO | | | FEDTKYTTLIAKLKSDGIPMYKRNVMILTNPVAAKK | |
| | | X | | | NVSINTVTYEWAPPVQNQALARQYMQMLPKEKQ | |
| | | | | | PVAGSEGAQYRKKQLAKQLPAHDQDPSKCHELS | |
| | | | | | PREVKEMEQFVKKYKSEALGVGDVKLPCEMDAQ | |
| | | | | | GPKQMNIPGGDRSTPAAVGAMEDKSAEHKRTQY | |
| | | | | | SCYCCKLSMKEGDPAIYAERAGYDKLWHPACFV | |
| | | | | | CSTCHELLVDMIYFWKNEKLYCGRHYCDSEKPR | |
| | | | | | CAGCDELIFSNEYTQAENQNWHLKHFCCFDCDSI | |
| | | | | | LAGEIYVMVNDKPVCKPCYVKNHAVVCQGCHNAI | |
| | | | | | DPEVQRVTYNNFSWHASTECFLCSCCSKCLIGQ | |
| | | | | | KFMPVEGMVFCSVECKKRMS | |
| 523 | JHUO | NIV | 3.28921441 | FBXO3 | MSPILGYWKIKGLVQPTRLLLEYLEEKYEEHLYER | Full Literal Sequence- |
| | 9258 | OIPI | 9 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAAMETETAPLTLES | |
| | | | | | LPTDPLLLILSFLDYRDLINCCYVSRRLSQLSSHDP | |
| | | | | | LWRRHCKKYWLISEEEKTQKNQCWKSLFIDTYSD | |
| | | | | | VGRYIDHYAAIKKAWDDLKKYLEPRCPRMVLSLK | |
| | | | | | EGAREEDLDAVEAQIGCKLPDDYRCSYRIHNGQK | |
| | | | | | LVVPGLLGSMALSNHYRSEDLLDVDTAAGGFQQ | |
| | | | | | RQGLKYCLPLTFCIHTGLSQYIAVEAAEGRNKNEV | |
| | | | | | FYQCPDQMARNPAAIDMFIIGATFTDWFTSYVKN | |
| | | | | | VVSGGFPIIRDQIFRYVHDPECVATTGDITVSVSTS | |
| | | | | | FLPELSSVHPPHYFFTYRIRIEMSKDALPEKACQL | |
| | | | | | DSRYWRITNAKGDVEEVQGPGVVGEFPIISPGRV | |
| | | | | | YEYTSCTTFSTTSGYMEGYYTFHFLYFKDKIFNVA | |
| | | | | | IPRFHMACPTFRVSIARLEMGPDEYEEMEEEEEE | |
| | | | | | EEEEDEDDDSADMDESDEDDEEERRRRVFDVPI | |
| | | | | | RRRRCSRLF | |
| 524 | JHUO | NIV | 3.28921441 | FBXO3 | MAAMETETAPLTLESLPTDPLLLILSFLDYRDLINC | Core Human ORF Only |
| | 9258 | OIPI | 9 | | CYVSRRLSQLSSHDPLWRRHCKKYWLISEEEKT | |
| | | _TO | | | QKNQCWKSLFIDTYSDVGRYIDHYAAIKKAWDDL | |
| | | X | | | KKYLEPRCPRMVLSLKEGAREEDLDAVEAQIGCK | |
| | | | | | LPDDYRCSYRIHNGQKLVVPGLLGSMALSNHYRS | |
| | | | | | EDLLDVDTAAGGFQQRQGLKYCLPLTFCIHTGLS | |
| | | | | | QYIAVEAAEGRNKNEVFYQCPDQMARNPAAIDM | |
| | | | | | FIIGATFTDWFTSYVKNVVSGGFPIIRDQIFRYVHD | |
| | | | | | PECVATTGDITVSVSTSFLPELSSVHPPHYFFTYRI | |
| | | | | | RIEMSKDALPEKACQLDSRYWRITNAKGDVEEVQ | |
| | | | | | GPGVVGEFPIISPGRVYEYTSCTTFSTTSGYMEG | |
| | | | | | YYTFHFLYFKDKIFNVAIPRFHMACPTFRVSIARLE | |
| | | | | | MGPDEYEEMEEEEEEEEEEDEDDDSADMDESD | |
| | | | | | EDDEEERRRRVFDVPIRRRRCSRLF | |
| 525 | JHUO | NIV | 0.47784019 | BUB3 | MSPILGYWKIKGLVQPTRLLLEYLEEKYEEHLYER | Full Literal Sequence- |
| | 9318 | OIPI | 7 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMTGSNEFKLNQPPE | |
| | | | | | DGISSVKFSPNTSQFLLVSSWDTSVRLYDVPANS | |
| | | | | | MRLKYQHTGAVLDCAFYDPTHAWSGGLDHQLK | |
| | | | | | MHDLNTDQENLVGTHDAPIRCVEYCPEVNVMVT | |
| | | | | | GSWDQTVKLWDPRTPCNAGTFSQPEKVYTLSVS | |
| | | | | | GDRLIVGTAGRRVLVWDLRNMGYVQQRRESSLK | |
| | | | | | YQTRCIRAFPNKQGYVLSSIEGRVAVEYLDPSPE | |
| | | | | | VQKKKYAFKCHRLKENNIEQIYPVNAISFHNIHNTF | |
| | | | | | ATGGSDGFVNIWDPFNKKRLCQFHRYPTSIASLA | |
| | | | | | FSNDGTTLAIASSYMYEMDDTEHPEDGIFIRQVTD | |
| | | | | | AETKPKSPCT | |
| 526 | JHUO | NIV | 0.47784019 | BUB3 | MTGSNEFKLNQPPEDGISSVKFSPNTSQFLLVSS | Core Human ORF Only |
| | 9318 | OIPI | 7 | | WDTSVRLYDVPANSMRLKYQHTGAVLDCAFYDP | |
| | | _TO | | | THAWSGGLDHQLKMHDLNTDQENLVGTHDAPIR | |
| | | X | | | CVEYCPEVNVMVTGSWDQTVKLWDPRTPCNAG | |
| | | | | | TFSQPEKVYTLSVSGDRLIVGTAGRRVLVWDLRN | |
| | | | | | MGYVQQRRESSLKYQTRCIRAFPNKQGYVLSSIE | |
| | | | | | GRVAVEYLDPSPEVQKKKYAFKCHRLKENNIEQI | |
| | | | | | YPVNAISFHNIHNTFATGGSDGFVNIWDPFNKKRL | |
| | | | | | CQFHRYPTSIASLAFSNDGTTLAIASSYMYEMDDT | |
| | | | | | EHPEDGIFIRQVTDAETKPKSPCT | |
| 527 | JHUO | NIV | 0.4345331 | PRSS3 | MSPILGYWKIKGLVQPTRLLLEYLEEKYEEHLYER | Full Literal Sequence- |
| | 9768 | OIPI | 14 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNPFLILAFVGAAVA | |
| | | | | | VPFDDDDKIVGGYTCEENSLPYQVSLNSGSHFCG | |
| | | | | | GSLISEQVWVSAAHCYKTRIQVRLGEHNIKVLEG | |
| | | | | | NEQFINAAKIIRHPKYNRDTLDNDIMLIKLSSPAVIN | |
| | | | | | ARVSTISLPTAPPAAGTECLISGWGNTLSFGADYP | |
| | | | | | DELKCLDAPVLTQAECKASYPGKITNSMFCVGFL | |
| | | | | | EGGKDSCQRDSGGPVVCNGQLQGVVSWGHGC | |
| | | | | | AWKNRPGVYTKVYNYVDWIKDTIAANS | |
| 528 | JHUO | NIV | 0.43453311 | PRSS3 | MNPFLILAFVGAAVAVPFDDDDKIVGGYTCEENSL | Core Human ORF Only |
| | 9768 | OIPI | 4 | | PYQVSLNSGSHFCGGSLISEQWVVSAAHCYKTRI | |
| | | _TO | | | QVRLGEHNIKVLEGNEQFINAAKIIRHPKYNRDTL | |
| | | X | | | DNDIMLIKLSSPAVINARVSTISLPTAPPAAGTECLI | |
| | | | | | SGWGNTLSFGADYPDELKCLDAPVLTQAECKAS | |
| | | | | | YPGKITNSMFCVGFLEGGKDSCQRDSGGPVVCN | |
| | | | | | GQLQGVVSWGHGCAWKNRPGVYTKVYNYVDWI | |
| | | | | | KDTIAANS | |
| 529 | JHUO | NIV | 2.52268036 | FAM96 | MSPILGYWKIKGLVQPTRLLLEYLEEKYEEHLYER | Full Literal Sequence- |
| | 9813 | OIPI | 2 | B | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMVGGGGVGGGLLEN | |
| | | | | | ANPLIYQRSGERPVTAGEEDEQVPDSIDAREIFDL | |
| | | | | | IRSINDPEHPLTLEELNVVEQVRVQVSDPESTVAV | |
| | | | | | AFTPTIPHCSMATLIGLSIKVKLLRSLPQRFKMDVH | |
| | | | | | ITPGTHASEHAVNKQLADKERVAAALENTHLLEVV | |
| | | | | | NQCLSARS | |
| 530 | JHUO | NIV | 2.52268036 | FAM96 | MVGGGGVGGGLLENANPLIYQRSGERPVTAGEE | Core Human ORF Only |
| | 9813 | OIPI | 2 | B | DEQVPDSIDAREIFDLIRSINDPEHPLTLEELNVVE | |
| | | _TO | | | QVRVQVSDPESTVAVAFTPTIPHCSMATLIGLSIK | |
| | | X | | | VKLLRSLPQRFKMDVHITPGTHASEHAVNKQLAD | |
| | | | | | KERVAAALENTHLLEVVNQCLSARS | |
| 531 | JHUO | NIV | 0.54122683 | ARTN | MSPILGYWKIKGLVQPTRLLLEYLEEKYEEHLYER | Full Literal Sequence- |
| | 9891 | OIPI | 8 | | DEGDKWRNKKFELGLEFPNLPYYIDGDVKLTQSM | (including GST-tag, 6- |
| | | | | | AIIRYIADKHNMLGGCPKERAEISMLEGAVLDIRYG | His leader, cloning |
| | | _TO | | | VSRIAYSKDFETLKVDFLSKLPEMLKMFEDRLCHK | adapters, core human |
| | | X | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMELGLGGLSTLSHCP | |
| | | | | | WPRQQAPLGLSAQPALWPTLAALALLSSVAEASL | |
| | | | | | GSAPRSPAPREGPPPVLASPAGHLPGGRTARWC | |
| | | | | | SGRARRPPPQPSRPAPPPPAPPSALPRGGRAAR | |
| | | | | | AGGPGSRARAAGARGCRLRSQLVPVRALGLGHR | |
| | | | | | SDELVRFRFCSGSCRRARSPHDLSLASLLGAGAL | |
| | | | | | RPPPGSRPVSQPCCRPTRYEAVSFMDVNSTWRT | |
| | | | | | VDRLSATACGCLG | |
| 532 | JHUO | NIV | 0.54122683 | ARTN | MELGLGGLSTLSHCPWPRQQAPLGLSAQPALWP | Core Human ORF Only |
| | 9891 | OIPI | 8 | | TLAALALLSSVAEASLGSAPRSPAPREGPPPVLAS | |
| | | _TO | | | PAGHLPGGRTARWCSGRARRPPPQPSRPAPPP | |
| | | X | | | PAPPSALPRGGRAARAGGPGSRARAAGARGCR | |
| | | | | | LRSQLVPVRALGLGHRSDELVRFRFCSGSCRRA | |
| | | | | | RSPHDLSLASLLGAGALRPPPGSRPVSQPCCRPT | |
| | | | | | RYEAVSFMDVNSTWRTVDRLSATACGCLG | |
| 533 | JHU1 | NIV | 5.95436770 | GDF9 | MSPILGYWKIKGLVQPTRLLLEYLEEKYEEHLYER | Full Literal Sequence- |
| | 1260 | OIPI | 5 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMARPNKFLLWFCCF | |
| | | | | | AWLCFPISLGSQASGGEAQIAASAELESGAMPWS | |
| | | | | | LLQHIDERDRAGLLPALFKVLSVGRGGSPRLQPD | |
| | | | | | SRALHYMKKLYKTYATKEGIPKSNRSHLYNTVRLF | |
| | | | | | TPCTRHKQAPGDQVTGILPSVELLFNLDRITTVEH | |
| | | | | | LLKSVLLYNINNSVSFSSAVKCVCNLMIKEPKSSS | |
| | | | | | RTLGRAPYSFTFNSQFEFGKKHKWIQIDVTSLLQP | |
| | | | | | LVASNKRSIHMSINFTCMKDQLEHPSAQNGLFNM | |
| | | | | | TLVSPSLILYLNDTSAQAYHSWYSLHYKRRPSQG | |
| | | | | | PDQERSLSAYPVGEEAAEDGRSSHHRHRRGQET | |
| | | | | | VSSELKKPLGPASFNLSEYFRQFLLPQNECELHD | |
| | | | | | FRLSFSQLKWDNWIVAPHRYNPRYCKGDCPRAV | |
| | | | | | GHRYGSPVHTMVQNIIYEKLDSSVPRPSCVPAKY | |
| | | | | | SPLSVLTIEPDGSIAYKEYEDMIATKCTCR | |
| 534 | JHU1 | NIV | 5.95436770 | GDF9 | MARPNKFLLWFCCFAWLCFPISLGSQASGGEAQI | Core Human ORF Only |
| | 1260 | OIPI | 5 | | AASAELESGAMPWSLLQHIDERDRAGLLPALFKV | |
| | | _TO | | | LSVGRGGSPRLQPDSRALHYMKKLYKTYATKEGI | |
| | | X | | | PKSNRSHLYNTVRLFTPCTRHKQAPGDQVTGILP | |
| | | | | | SVELLFNLDRITTVEHLLKSVLLYNINNSVSFSSAV | |
| | | | | | KCVCNLMIKEPKSSSRTLGRAPYSFTFNSQFEFG | |
| | | | | | KKHKWIQIDVTSLLQPLVASNKRSIHMSINFTCMK | |
| | | | | | DQLEHPSAQNGLFNMTLVSPSLILYLNDTSAQAY | |
| | | | | | HSWYSLHYKRRPSQGPDQERSLSAYPVGEEAAE | |
| | | | | | DGRSSHHRHRRGQETVSSELKKPLGPASFNLSE | |
| | | | | | YFRQFLLPQNECELHDFRLSFSQLKWDNWIVAPH | |
| | | | | | RYNPRYCKGDCPRAVGHRYGSPVHTMVQNIIYE | |
| | | | | | KLDSSVPRPSCVPAKYSPLSVLTIEPDGSIAYKEY | |
| | | | | | EDMIATKCTCR | |
| 535 | JHU1 | NIV | 0.22038930 | NOXA1 | MSPILGYWKIKGLVQPTRLLLEYLEEKYEEHLYER | Full Literal Sequence- |
| | 2219 | OIPI | 8 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASLGDLVRAWHLG | |
| | | | | | AQAVDRGDWARALHLFSGVPAPPARLCFNAGCV | |
| | | | | | HLLAGDPEAALRAFDQAVTKDTCMAVGFFQRGV | |
| | | | | | ANFQLARFQEALSDFWLALEQLRGHAAIDYTQLG | |
| | | | | | LRFKLQAWEVLHNVASAQCQLGLWTEAASSLRE | |
| | | | | | AMSKWPEGSLNGLDSALDQVQRRGSLPPRQVP | |
| | | | | | RGEVFRPHRWHLKHLEPVDFLGKAKVVASAIPDD | |
| | | | | | QGWGVRPQQPQGPGANHDARSLIMDSPRAGTH | |
| | | | | | QGPLDAETEVGADRCTSTAYQEQRPQVEQVGK | |
| | | | | | QAPLSPGLPAMGGPGPGPCEDPAGAGGAGAGG | |
| | | | | | SEPLVTVTVQCAFTVALRARRGADLSSLRALLGQ | |
| | | | | | ALPHQAQLGQLSYLAPGEDGHWVPIPEEESLQR | |
| | | | | | AWQDAAACPRGLQLQCRGAGGRPVLYQVVAQH | |
| | | | | | SYSAQGPEDLGFRQGDTVDVLCEEPDVPLAVDQ | |
| | | | | | AWLEGHCDGRIGIFPKCFVVPAGPRMSGAPGRL | |
| | | | | | PRSQQGDQP | |
| 536 | JHU1 | NIV | 0.22038930 | NOXA1 | MASLGDLVRAWHLGAQAVDRGDWARALHLFSG | Core Human ORF Only |
| | 2219 | OIPI | 8 | | VPAPPARLCFNAGCVHLLAGDPEAALRAFDQAVT | |
| | | _TO | | | KDTCMAVGFFQRGVANFQLARFQEALSDFWLAL | |
| | | X | | | EQLRGHAAIDYTQLGLRFKLQAWEVLHNVASAQC | |
| | | | | | QLGLWTEAASSLREAMSKWPEGSLNGLDSALDQ | |
| | | | | | VQRRGSLPPRQVPRGEVFRPHRWHLKHLEPVDF | |
| | | | | | LGKAKVVASAIPDDQGWGVRPQQPQGPGANHD | |
| | | | | | ARSLIMDSPRAGTHQGPLDAETEVGADRCTSTAY | |
| | | | | | QEQRPQVEQVGKQAPLSPGLPAMGGPGPGPCE | |
| | | | | | DPAGAGGAGAGGSEPLVTVTVQCAFTVALRARR | |
| | | | | | GADLSSLRALLGQALPHQAQLGQLSYLAPGEDG | |
| | | | | | HVWPIPEEESLQRAWQDAAACPRGLQLQCRGA | |
| | | | | | GGRPVLYQVVAQHSYSAQGPEDLGFRQGDTVD | |
| | | | | | VLCEEPDVPLAVDQAWLEGHCDGRIGIFPKCFVV | |
| | | | | | PAGPRMSGAPGRLPRSQQGDQP | |
| 537 | JHU1 | NIV | 0.79926950 | SLC25 | MSPILGYWKIKGLVQPTRLLLEYLEEKYEEHLYER | Full Literal Sequence- |
| | 2996 | OIPI | 4 | A43 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMATWRRDGRLTGG | |
| | | | | | QRLLCAGLAGTLSLSLTAPLELATVLAQVGVVRG | |
| | | | | | HARGPWATGHRVWRAEGLRALWKGNAVACLRL | |
| | | | | | FPCSAVQLAAYRKFVVLFTDDLGHISQWSSIMAG | |
| | | | | | SLAGMVSTIVTYPTDLIKTRLIMQNILEPSYRGLLH | |
| | | | | | AFSTIYQQEGFLALYRGVSLTVVGALPFSAGSLLV | |
| | | | | | YMNLEKIWNGPRDQFSLPQNFANVCLAAAVTQTL | |
| | | | | | SFPFETVKRKMQAQSPYLPHSGGVDVHFSGAVD | |
| | | | | | CFRQIVKAQGVLGLWNGLTANLLKIVPYFGIMFST | |
| | | | | | FEFCKRICLYQNGYILSPLSYKLTPGVDQSLQPQE | |
| | | | | | LRELKKFFKTRKLKPKKPTL | |
| 538 | JHU1 | NIV | 0.79926950 | SLC25 | MATWRRDGRLTGGQRLLCAGLAGTLSLSLTAPL | Core Human ORF Only |
| | 2996 | OIPI | 4 | A43 | ELATVLAQVGVVRGHARGPWATGHRVWRAEGL | |
| | | _TO | | | RALWKGNAVACLRLFPCSAVQLAAYRKFVVLFTD | |
| | | X | | | DLGHISQWSSIMAGSLAGMVSTIVTYPTDLIKTRLI | |
| | | | | | MQNILEPSYRGLLHAFSTIYQQEGFLALYRGVSLT | |
| | | | | | VVGALPFSAGSLLVYMNLEKIWNGPRDQFSLPQN | |
| | | | | | FANVCLAAAVTQTLSFPFETVKRKMQAQSPYLPH | |
| | | | | | SGGVDVHFSGAVDCFRQIVKAQGVLGLWNGLTA | |
| | | | | | NLLKIVPYFGIMFSTFEFCKRICLYQNGYILSPLSY | |
| | | | | | KLTPGVDQSLQPQELRELKKFFKTRKLKPKKPTL | |
| 539 | JHU1 | NIV | 2.72734911 | ACTRT | MSPILGYWKIKGLVQPTRLLLEYLEEKYEEHLYER | Full Literal Sequence- |
| | 3500 | OIPI | 1 | 2 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMFNPHALDSPAVIFD | |
| | | | | | NGSGFCKAGLSGEFGPRHMVSSIVGHLKFQAPS | |
| | | | | | AEANQKKYFVGEEALYKQEALQLHSPFERGLITG | |
| | | | | | WDDVERLWKHLFEWELGVKPSDQPLLATEPSLN | |
| | | | | | PRENREKMAEVMFENFGVPAFYLSDQAVLALYA | |
| | | | | | SACVTGLVVDSGDAVTCTVPIFEGYSLPHAVTKL | |
| | | | | | HVAGRDITELLMQLLLASGHTFPCQLDKGLVDDIK | |
| | | | | | KKLCYVALEPEKELSRRPEEVLREYKLPDGNIISL | |
| | | | | | GDPLHQAPEALFVPQQLGSQSPGLSNMVSSSITK | |
| | | | | | CDTDIQKILFGEIVLSGGTTLFHGLDDRLLKELEQL | |
| | | | | | ASKDTPIKITAPPDRWFSTWIGASIVTSLSSFKQM | |
| | | | | | WVTAADFKEFGTSVVQRRCF | |
| 540 | JHU1 | NIV | 2.72734911 | ACTRT | MFNPHALDSPAVIFDNGSGFCKAGLSGEFGPRH | Core Human ORF Only |
| | 3500 | OIPI | 1 | 2 | MVSSIVGHLKFQAPSAEANQKKYFVGEEALYKQE | |
| | | _TO | | | ALQLHSPFERGLITGWDDVERLWKHLFEWELGV | |
| | | X | | | KPSDQPLLATEPSLNPRENREKMAEVMFENFGV | |
| | | | | | PAFYLSDQAVLALYASACVTGLVVDSGDAVTCTV | |
| | | | | | PIFEGYSLPHAVTKLHVAGRDITELLMQLLLASGH | |
| | | | | | TFPCQLDKGLVDDIKKKLCYVALEPEKELSRRPEE | |
| | | | | | VLREYKLPDGNIISLGDPLHQAPEALFVPQQLGSQ | |
| | | | | | SPGLSNMVSSSITKCDTDIQKILFGEIVLSGGTTLF | |
| | | | | | HGLDDRLLKELEQLASKDTPIKITAPPDRWFSTWI | |
| | | | | | GASIVTSLSSFKQMVWTAADFKEFGTSVVQRRCF | |
| 541 | JHU1 | NIV | 1.31474194 | ACVR2 | MSPILGYWKIKGLVQPTRLLLEYLEEKYEEHLYER | Full Literal Sequence- |
| | 3691 | OIPI | 2 | A | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGAAAKLAFAVFLIS | |
| | | | | | CSSGAILGRSETQECLFFNANWEKDRTNQTGVE | |
| | | | | | PCYGDKDKRRHCFATWKNISGSIEIVKQGCWLDD | |
| | | | | | INCYDRTDCVEKKDSPEVYFCCCEGNMCNEKFS | |
| | | | | | YFPEMEVTQPTSNPVTPKPPYYNILLYSLVPLMLI | |
| | | | | | AGIVICAFVWYRHHKMAYPPVLVPTQDPGPPPPS | |
| | | | | | PLLGLKPLQLLEVKARGRFGCVWKAQLLNEYVAV | |
| | | | | | KIFPIQDKQSWQNEYEVYSLPGMKHENILQFIGAE | |
| | | | | | KRGTSVDVDLWLITAFHEKGSLSDFLKANVVSWN | |
| | | | | | ELCHIAETMARGLAYLHEDIPGLKDGHKPAISHRD | |
| | | | | | IKSKNVLLKNNLTACIADFGLALKFEAGKSAGDTH | |
| | | | | | GQVGTRRYMAPEVLEGAINFQRDAFLRIDMYAM | |
| | | | | | GLVLWELASRCTAADGPVDEYMLPFEEEIGQHPS | |
| | | | | | LEDMQEVVVHKKKRPVLRDYWQKHAGMAMLCE | |
| | | | | | TIEECWDHDAEARLSAGCVGERITQMQRLTNIITT | |
| | | | | | EDIVTVVTMVTNVDFPPKESSL | |
| 542 | JHU1 | NIV | 1.31474194 | ACVR2 | MGAAAKLAFAVFLISCSSGAILGRSETQECLFFNA | Core Human ORF Only |
| | 3691 | OIPI | 2 | A | NWEKDRTNQTGVEPCYGDKDKRRHCFATWKNIS | |
| | | _TO | | | GSIEIVKQGCWLDDINCYDRTDCVEKKDSPEVYF | |
| | | X | | | CCCEGNMCNEKFSYFPEMEVTQPTSNPVTPKPP | |
| | | | | | YYNILLYSLVPLMLIAGIVICAFVWYRHHKMAYPPV | |
| | | | | | LVPTQDPGPPPPSPLLGLKPLQLLEVKARGRFGC | |
| | | | | | VWKAQLLNEYVAVKIFPIQDKQSWQNEYEVYSLP | |
| | | | | | GMKHENILQFIGAEKRGTSVDVDLWLITAFHEKGS | |
| | | | | | LSDFLKANVVSWNELCHIAETMARGLAYLHEDIP | |
| | | | | | GLKDGHKPAISHRDIKSKNVLLKNNLTACIADFGL | |
| | | | | | ALKFEAGKSAGDTHGQVGTRRYMAPEVLEGAINF | |
| | | | | | QRDAFLRIDMYAMGLVLWELASRCTAADGPVDE | |
| | | | | | YMLPFEEEIGQHPSLEDMQEVVVHKKKRPVLRDY | |
| | | | | | WQKHAGMAMLCETIEECWDHDAEARLSAGCVG | |
| | | | | | ERITQMQRLTNIITTEDIVTVVTMVTNVDFPPKESS | |
| | | | | | L | |
| 543 | JHU1 | NIV | 3.77959067 | CBFA2 | MSPILGYWKIKGLVQPTRLLLEYLEEKYEEHLYER | Full Literal Sequence- |
| | 4629 | OIPI | 6 | T3 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMPASRLRDRAASSA | |
| | | | | | SGSTCGSMSQTHPVLESGLLASAGCSAPRGPRK | |
| | | | | | GGPAPVDRKAKASAMPDSPAEVKTQPRSTPPSM | |
| | | | | | PPPPPAASQGATRPPSFTPHTLMNGSSHSPTAIN | |
| | | | | | GAPCTPNGFSNGPATSSTASLSTQHLPPACGAR | |
| | | | | | QLSKLKRFLTTLQQFGSDISPEIGERVRTLVLGLV | |
| | | | | | NSTLTIEEFHSKLQEATNFPLRPFVIPFLKANLPLL | |
| | | | | | QRELLHCARLAKQTPAQYLAQHEQLLLDASASSP | |
| | | | | | IDSSELLLEVNENGKRRTPDRTKENGSDRDPLHP | |
| | | | | | QRYSPSNGPPQPTPPPHYRLEDIAMAHHFRDAY | |
| | | | | | RHPDPRELRERHRPLVVPGSRQEEVIDHKLTERE | |
| | | | | | WAEEWKHLNNLLNCIMDMVEKTRRSLTVLRRCQ | |
| | | | | | EADREELNHWARRYSDAEDTKKGPAPAAARPRS | |
| | | | | | SSAGPEGPQLDVPREFLPRTLTGYVPEDIWRKAE | |
| | | | | | EAVNEVKRQAMSELQKAVSDAERKAHELITTERA | |
| | | | | | KMERALAEAKRQASEDALTVINQQEDSSESCWN | |
| | | | | | CGRKASETCSGCNAARYCGSFCQHRDWEKHHH | |
| | | | | | VCGQSLQGPTAVVADPVPGPPEAAHSLGPSLPV | |
| | | | | | GAASPSEAGSAGPSRPGSPSPPGPLDTVPR | |
| 544 | JHU1 | NIV | 3.77959067 | CBFA2 | MPASRLRDRAASSASGSTCGSMSQTHPVLESGL | Core Human ORF Only |
| | 4629 | OIPI | 6 | T3 | LASAGCSAPRGPRKGGPAPVDRKAKASAMPDSP | |
| | | _TO | | | AEVKTQPRSTPPSMPPPPPAASQGATRPPSFTP | |
| | | X | | | HTLMNGSSHSPTAINGAPCTPNGFSNGPATSSTA | |
| | | | | | SLSTQHLPPACGARQLSKLKRFLTTLQQFGSDISP | |
| | | | | | EIGERVRTLVLGLVNSTLTIEEFHSKLQEATNFPLR | |
| | | | | | PFVIPFLKANLPLLQRELLHCARLAKQTPAQYLAQ | |
| | | | | | HEQLLLDASASSPIDSSELLLEVNENGKRRTPDRT | |
| | | | | | KENGSDRDPLHPQRYSPSNGPPQPTPPPHYRLE | |
| | | | | | DIAMAHHFRDAYRHPDPRELRERHRPLVVPGSR | |
| | | | | | QEEVIDHKLTEREWAEEWKHLNNLLNCIMDMVEK | |
| | | | | | TRRSLTVLRRCQEADREELNHWARRYSDAEDTK | |
| | | | | | KGPAPAAARPRSSSAGPEGPQLDVPREFLPRTLT | |
| | | | | | GYVPEDIWRKAEEAVNEVKRQAMSELQKAVSDA | |
| | | | | | ERKAHELITTERAKMERALAEAKRQASEDALTVIN | |
| | | | | | QQEDSSESCWNCGRKASETCSGCNAARYCGSF | |
| | | | | | CQHRDWEKHHHVCGQSLQGPTAVVADPVPGPP | |
| | | | | | EAAHSLGPSLPVGAASPSEAGSAGPSRPGSPSP | |
| | | | | | PGPLDTVPR | |
| 545 | JHU1 | NIV | 0.62805760 | MFAP5 | MSPILGYWKIKGLVQPTRLLLEYLEEKYEEHLYER | Full Literal Sequence- |
| | 4662 | OIPI | 6 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSLLGPKVLLFLAAFI | |
| | | | | | ITSDWIPLGVNSQRGDDVTQATPETFTEDPNLVN | |
| | | | | | DPATDETVLAVLADIAPSTDDLASLSEKNTTAECW | |
| | | | | | DEKFTCTRLYSVHRPVKQCIHQLCFTSLRRMYIVN | |
| | | | | | KEICSRLVCKEHEAMKDELCRQMAGLPPRRLRR | |
| | | | | | SNYFRLPPCENVDLQRPNGL | |
| 546 | JHU1 | NIV | 0.62805760 | MFAP5 | MSLLGPKVLLFLAAFIITSDWIPLGVNSQRGDDVT | Core Human ORF Only |
| | 4662 | OIPI | 6 | | QATPETFTEDPNLVNDPATDETVLAVLADIAPSTD | |
| | | _TO | | | DLASLSEKNTTAECWDEKFTCTRLYSVHRPVKQC | |
| | | X | | | IHQLCFTSLRRMYIVNKEICSRLVCKEHEAMKDEL | |
| | | | | | CRQMAGLPPRRLRRSNYFRLPPCENVDLQRPNG | |
| | | | | | L | |
| 547 | JHU1 | NIV | 2.87536856 | ZHX3 | MSPILGYWKIKGLVQPTRLLLEYLEEKYEEHLYER | Full Literal Sequence- |
| | 4706 | OIPI | 5 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMASKRKSTTPCMIPV | |
| | | | | | KTVVLQDASMEAQPAETLPEGPQQDLPPEASAA | |
| | | | | | SSEAAQNPSSTDGSTLANGHRSTLDGYLYSCKY | |
| | | | | | CDFRSHDMTQFVGHMNSEHTDFNKDPTFVCSG | |
| | | | | | CSFLAKTPEGLSLHNATCHSGEASFVWNVAKPD | |
| | | | | | NHVVVEQSIPESTSTPDLAGEPSAEGADGQAEIIIT | |
| | | | | | KTPIMKIMKGKAEAKKIHTLKENVPSQPVGEALPK | |
| | | | | | LSTGEMEVREGDHSFINGAVPVSQASASSAKNP | |
| | | | | | HAANGPLIGTVPVLPAGIAQFLSLQQQPPVHAQH | |
| | | | | | HVHQPLPTAKALPKVMIPLSSIPTYNAAMDSNSFL | |
| | | | | | KNSFHKFPYPTKAELCYLTVVTKYPEEQLKIWFTA | |
| | | | | | QRLKQGISWSPEEIEDARKKMFNTVIQSVPQPTIT | |
| | | | | | VLNTPLVASAGNVQHLIQAALPGHVVGQPEGTGG | |
| | | | | | GLLVTQPLMANGLQATSSPLPLTVTSVPKQPGVA | |
| | | | | | PINTVCSNTTSAVKVVNAAQSLLTACPSITSQAFL | |
| | | | | | DASIYKNKKSHEQLSALKGSFCRNQFPGQSEVEH | |
| | | | | | LTKVTGLSTREVRKWFSDRRYHCRNLKGSRAMIP | |
| | | | | | GDHSSIIIDSVPEVSFSPSSKVPEVTCIPTTATLAT | |
| | | | | | HPSAKRQSWHQTPDFTPTKYKERAPEQLRALES | |
| | | | | | SFAQNPLPLDEEMDRLRSETKMTRREIDSWFSER | |
| | | | | | RKKVNAEETKKAEENASQEEEEAAEDEGGEEDL | |
| | | | | | ASELRVSGENGSLEMPSSHILAERKVSPIKINLKN | |
| | | | | | LRVTEANGRNEIPGLGACDPEDDESNKLAEQLPG | |
| | | | | | KVSCKKTAQQRHLLRQLFVQTQWPSNQDYDSIM | |
| | | | | | AQTGLPRPEVVRWFGDSRYALKNGQLKWYEDY | |
| | | | | | KRGNFPPGLLVIAPGNRELLQDYYMTHKMLYEED | |
| | | | | | LQNLCDKTQMSSQQKQTEFDLINVKDWPVWETA | |
| | | | | | CHVEEPNPTLCCHMPFPCPAAGHLGELPESSQT | |
| | | | | | AQSLPLPSACPPPSKQQARWGSHQFFLPQCRTF | |
| | | | | | PLPSNG | |
| 548 | JHU1 | NIV | 2.87536856 | ZHX3 | MASKRKSTTPCMIPVKTVVLQDASMEAQPAETLP | Core Human ORF Only |
| | 4706 | OIPI | 5 | | EGPQQDLPPEASAASSEAAQNPSSTDGSTLANG | |
| | | _TO | | | HRSTLDGYLYSCKYCDFRSHDMTQFVGHMNSEH | |
| | | X | | | TDFNKDPTFVCSGCSFLAKTPEGLSLHNATCHSG | |
| | | | | | EASFVWNVAKPDNHVVVEQSIPESTSTPDLAGEP | |
| | | | | | SAEGADGQAEIIITKTPIMKIMKGKAEAKKIHTLKE | |
| | | | | | NVPSQPVGEALPKLSTGEMEVREGDHSFINGAVP | |
| | | | | | VSQASASSAKNPHAANGPLIGTVPVLPAGIAQFLS | |
| | | | | | LQQQPPVHAQHHVHQPLPTAKALPKVMIPLSSIPT | |
| | | | | | YNAAMDSNSFLKNSFHKFPYPTKAELCYLTVVTK | |
| | | | | | YPEEQLKIWFTAQRLKQGISWSPEEIEDARKKMF | |
| | | | | | NTVIQSVPQPTITVLNTPLVASAGNVQHLIQAALP | |
| | | | | | GHVVGQPEGTGGGLLVTQPLMANGLQATSSPLP | |
| | | | | | LTVTSVPKQPGVAPINTVCSNTTSAVKVVNAAQS | |
| | | | | | LLTACPSITSQAFLDASIYKNKKSHEQLSALKGSF | |
| | | | | | CRNQFPGQSEVEHLTKVTGLSTREVRKWFSDRR | |
| | | | | | YHCRNLKGSRAMIPGDHSSIIIDSVPEVSFSPSSK | |
| | | | | | VPEVTCIPTTATLATHPSAKRQSWHQTPDFTPTK | |
| | | | | | YKERAPEQLRALESSFAQNPLPLDEEMDRLRSET | |
| | | | | | KMTRREIDSWFSERRKKVNAEETKKAEENASQE | |
| | | | | | EEEAAEDEGGEEDLASELRVSGENGSLEMPSSHI | |
| | | | | | LAERKVSPIKINLKNLRVTEANGRNEIPGLGACDP | |
| | | | | | EDDESNKLAEQLPGKVSCKKTAQQRHLLRQLFV | |
| | | | | | QTQWPSNQDYDSIMAQTGLPRPEVVRWFGDSR | |
| | | | | | YALKNGQLKWYEDYKRGNFPPGLLVIAPGNRELL | |
| | | | | | QDYYMTHKMLYEEDLQNLCDKTQMSSQQKQTEF | |
| | | | | | DLINVKDWPVWETACHVEEPNPTLCCHMPFPCP | |
| | | | | | AAGHLGELPESSQTAQSLPLPSACPPPSKQQAR | |
| | | | | | WGSHQFFLPQCRTFPLPSNG | |
| 549 | JHU1 | NIV | 1.4550091 | KBTBD | MSPILGYWKIKGLVQPTRLLLEYLEEKYEEHLYER | Full Literal Sequence- |
| | 4758 | OIPI | 6 | 12 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMIEETSFGKSYAQLN | |
| | | | | | FENSMLWMDLPDEEPDRLSNKLLQYDPSQDQW | |
| | | | | | SVRAPMKYSKYRFSTAVVNSEIYVLGGIGCVGQD | |
| | | | | | KGQVRKCLDVVEIYNPDGDFWREGPPMPSPLLS | |
| | | | | | LRTNSTNAGAVDGKLYVCGGFHGADRHEVISKEI | |
| | | | | | LELDPWENQWNVVAINVLMHDSYDVCLVARMNP | |
| | | | | | RDLIPPPSDLVEEGNEH | |
| 550 | JHU1 | NIV | 1.4550091 | KBTBD | MIEETSFGKSYAQLNFENSMLWMDLPDEEPDRL | Core Human ORF Only |
| | 4758 | OIPI | 6 | 12 | SNKLLQYDPSQDQWSVRAPMKYSKYRFSTAVVN | |
| | | _TO | | | SEIYVLGGIGCVGQDKGQVRKCLDVVEIYNPDGD | |
| | | X | | | FWREGPPMPSPLLSLRTNSTNAGAVDGKLYVCG | |
| | | | | | GFHGADRHEVISKEILELDPWENQWNVVAINVLM | |
| | | | | | HDSYDVCLVARMNPRDLIPPPSDLVEEGNEH | |
| 551 | JHU1 | NIV | 0.11872163 | PTEN | MSPILGYWKIKGLVQPTRLLLEYLEEKYEEHLYER | Full Literal Sequence- |
| | 5357 | OIPI | 4 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMGFPAERLEGVYRN | |
| | | | | | NIDDVVRFLDSKHKNHYKIHNLCAERHYDTAKSN | |
| | | | | | YRVAQYPFEDHNPPQLELIKPFCEDLDQWLSEDD | |
| | | | | | NHVAAIHCKAGKGRTGIMIYAYLLHRGKFLKAQEA | |
| | | | | | LDFYGEVRTRDKKGVTIPSQRRYVYYYSYLVKNH | |
| | | | | | VDYRPVALLFHKMMFETIPMFSGGTCNPQFVVC | |
| | | | | | QLKVKMYSSNSGPTRWEDKFMYFEFPQPLPVCG | |
| | | | | | GIKVEFFHKQNKMLKKDKMFHFWVNTFFIPGPEE | |
| | | | | | TSEKVENGSLCDQEIDSICSIERADNDKEYLVLTLT | |
| | | | | | KNDLDKANKDKANRYFSPNFKVKLYFTKTVEEPS | |
| | | | | | NPEASSSTSVTPDVSDNEPDHYRYSDTTDSDPE | |
| | | | | | NEPFDEDQHTQITKV | |
| 552 | JHU1 | NIV | 0.11872163 | PTEN | MGFPAERLEGVYRNNIDDVVRFLDSKHKNHYKIH | Core Human ORF Only |
| | 5357 | OIPI | 4 | | NLCAERHYDTAKSNYRVAQYPFEDHNPPQLELIK | |
| | | _TO | | | PFCEDLDQWLSEDDNHVAAIHCKAGKGRTGIMIY | |
| | | X | | | AYLLHRGKFLKAQEALDFYGEVRTRDKKGVTIPS | |
| | | | | | QRRYVYYYSYLVKNHVDYRPVALLFHKMMFETIP | |
| | | | | | MFSGGTCNPQFVVCQLKVKMYSSNSGPTRWED | |
| | | | | | KFMYFEFPQPLPVCGGIKVEFFHKQNKMLKKDKM | |
| | | | | | FHFWVNTFFIPGPEETSEKVENGSLCDQEIDSICS | |
| | | | | | IERADNDKEYLVLTLTKNDLDKANKDKANRYFSP | |
| | | | | | NFKVKLYFTKTVEEPSNPEASSSTSVTPDVSDNE | |
| | | | | | PDHYRYSDTTDSDPENEPFDEDQHTQITKV | |
| 553 | JHU1 | NIV | 0.12088619 | DYNC1 | MSPILGYWKIKGLVQPTRLLLEYLEEKYEEHLYER | Full Literal Sequence- |
| | 6272 | OIPI | 6 | H1 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMISKMLKMQMLEDE | |
| | | | | | DDLAYAETEKKTRTDSTSDGRPAWMRTLHTTAS | |
| | | | | | NWLHLIPQTLSHLKRTVENIKDPLFRFFEREVKMG | |
| | | | | | AKLLQDVRQDLADVVQVCEGKKKQTNYLRTLINE | |
| | | | | | LVKGILPRSWSHYTVPAGMTVIQWVSDFSERIKQ | |
| | | | | | LQNISLAAASGGAKELKVKALLTSLGWSAAVLGW | |
| | | | | | GGSGSGEKHRAQV | |
| 554 | JHU1 | NIV | 0.12088619 | DYNC1 | MISKMLKMQMLEDEDDLAYAETEKKTRTDSTSDG | Core Human ORF Only |
| | 6272 | OIPI | 6 | H1 | RPAWMRTLHTTASNWLHLIPQTLSHLKRTVENIK | |
| | | _TO | | | DPLFRFFEREVKMGAKLLQDVRQDLADVVQVCE | |
| | | X | | | GKKKQTNYLRTLINELVKGILPRSWSHYTVPAGM | |
| | | | | | TVIQWVSDFSERIKQLQNISLAAASGGAKELKVKA | |
| | | | | | LLTSLGWSAAVLGWGGSGSGEKHRAQV | |
| 555 | JHU1 | NIV | 3.54824635 | PRAME | MSPILGYWKIKGLVQPTRLLLEYLEEKYEEHLYER | Full Literal Sequence- |
| | 7573 | OIPI | 7 | F3 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMSLQAPRRLLELAG | |
| | | | | | QSLLGDQALAISILDELPRELFPPLFVEAFTSRRCE | |
| | | | | | VLKVMVQAWPFPCLPLGSLMKTPDLEILHYVVDGI | |
| | | | | | DCLLAQKVRPRRRKLQVLELRDVDENFWTIWSG | |
| | | | | | ARPLSCSPEAMSKRQTVEDCPRTGEKQPLKVFM | |
| | | | | | DVCLKEKFMDEDLSFFSGWVQHRRGSVHLCCTK | |
| | | | | | VVNYSMSILNFRNILETVYPDSIQVLEIWNMCWPC | |
| | | | | | MIVEFIRYLSQMRNLRKLFISDGCRYLLSSDSQEQ | |
| | | | | | LVAEFSSVLLRLEYLQMLYVRRVCFFRGHLDQLIR | |
| | | | | | CLRSPLETLALTYGFLEKVDLKCLPRYPSLSQLKQ | |
| | | | | | LNLSHGALRFIRLEPLRALLEKVAATLQTLFLVDC | |
| | | | | | GIRDSKLRVILPALSCCSNLTTFCFHGNDTSMDGL | |
| | | | | | KDLLRHTGRLSNLSLETYPAPRESLDDRGRVISEL | |
| | | | | | LTPLQAELMRILREVREPKRIFFGPVSCPCCGTSP | |
| | | | | | TEQLEFNFCLWGRPANPAFLYKVVKASS | |
| 556 | JHU1 | NIV | 3.54824635 | PRAME | MSLQAPRRLLELAGQSLLGDQALAISILDELPREL | Core Human ORF Only |
| | 7573 | OIPI | 7 | F3 | FPPLFVEAFTSRRCEVLKVMVQAWPFPCLPLGSL | |
| | | _TO | | | MKTPDLEILHYVVDGIDCLLAQKVRPRRRKLQVLE | |
| | | X | | | LRDVDENFWTIWSGARPLSCSPEAMSKRQTVED | |
| | | | | | CPRTGEKQPLKVFMDVCLKEKFMDEDLSFFSGW | |
| | | | | | VQHRRGSVHLCCTKVVNYSMSILNFRNILETVYP | |
| | | | | | DSIQVLEIWNMCWPCMIVEFIRYLSQMRNLRKLFI | |
| | | | | | SDGCRYLLSSDSQEQLVAEFSSVLLRLEYLQMLY | |
| | | | | | VRRVCFFRGHLDQLIRCLRSPLETLALTYGFLEKV | |
| | | | | | DLKCLPRYPSLSQLKQLNLSHGALRFIRLEPLRAL | |
| | | | | | LEKVAATLQTLFLVDCGIRDSKLRVILPALSCCSNL | |
| | | | | | TTFCFHGNDTSMDGLKDLLRHTGRLSNLSLETYP | |
| | | | | | APRESLDDRGRVISELLTPLQAELMRILREVREPK | |
| | | | | | RIFFGPVSCPCCGTSPTEQLEFNFCLWGRPA | |
| 557 | JHU1 | NIV | 3.75601289 | DDX23 | MSPILGYWKIKGLVQPTRLLLEYLEEKYEEHLYER | Full Literal Sequence- |
| | 8252 | OIPI | 1 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGELADKKDRDAS | |
| | | | | | PSKEERKRSRTPDRERDRDRDRKSSPSKDRKRH | |
| | | | | | RSRDRRRGGSRSRSRSRSKSAERERRHKERER | |
| | | | | | DKERDRNKKDRDRDKDGHRRDKDRKRSSLSPG | |
| | | | | | RGKDFKSRKDRDSKKDEEDEHGDKKPKAQPLSL | |
| | | | | | EELLAKKKAEEEAEAKPKFLSKAEREAEALKRRQ | |
| | | | | | QEVEERQRMLEEERKKRKQFQDLGRKMLEDPQ | |
| | | | | | ERERRERRERMERETNGNEDEEGRQKIREEKDK | |
| | | | | | SKELHAIKERYLGGIKKRRRTRHLNDRKFVFEWD | |
| | | | | | ASEDTSIDYNPLYKERHQVQLLGRGFIAGIDLKQQ | |
| | | | | | KREQSRFYGDLMEKRRTLEEKEQEEARLRKLRK | |
| | | | | | KEAKQRWDDRHWSQKKLDEMTDRDWRIFREDY | |
| | | | | | SITTKGGKIPNPIRSWKDSSLPPHILEVIDKCGYKE | |
| | | | | | PTPIQRQAIPIGLQNRDIIGVAETGSGKTAAFLIPLL | |
| | | | | | VWITTLPKIDRIEESDQGPYAIILAPTRELAQQIEEE | |
| | | | | | TIKFGKPLGIRTVAVIGGISREDQGFRLRMGCEIVI | |
| | | | | | ATPGRLIDVLENRYLVLSRCTYVVLDEADRMIDM | |
| | | | | | GFEPDVQKILEHMPVSNQKPDTDEAEDPEKMLA | |
| | | | | | NFESGKHKYRQTVMFTATMPPAVERLARSYLRR | |
| | | | | | PAVVYIGSAGKPHERVEQKVFLMSESEKRKKLLAI | |
| | | | | | LEQGFDPPIIIFVNQKKGCDVLAKSLEKMGYNACT | |
| | | | | | LHGGKGQEQREFALSNLKAGAKDILVATDVAGRG | |
| | | | | | IDIQDVSMVVNYDMAKNIEDYIHRIGRTGRAGKSG | |
| | | | | | VAITFLTKEDSAVFYELKQAILESPVSSCPPELANH | |
| | | | | | PDAQHKPGTILTKKRREETIFANPAFLYKVVKASS | |
| 558 | JHU1 | NIV | 3.75601289 | DDX23 | MAGELADKKDRDASPSKEERKRSRTPDRERDRD | Core Human ORF Only |
| | 8252 | OIPI | 1 | | RDRKSSPSKDRKRHRSRDRRRGGSRSRSRSRS | |
| | | _TO | | | KSAERERRHKERERDKERDRNKKDRDRDKDGH | |
| | | X | | | RRDKDRKRSSLSPGRGKDFKSRKDRDSKKDEED | |
| | | | | | EHGDKKPKAQPLSLEELLAKKKAEEEAEAKPKFL | |
| | | | | | SKAEREAEALKRRQQEVEERQRMLEEERKKRKQ | |
| | | | | | FQDLGRKMLEDPQERERRERRERMERETNGNE | |
| | | | | | DEEGRQKIREEKDKSKELHAIKERYLGGIKKRRRT | |
| | | | | | RHLNDRKFVFEWDASEDTSIDYNPLYKERHQVQL | |
| | | | | | LGRGFIAGIDLKQQKREQSRFYGDLMEKRRTLEE | |
| | | | | | KEQEEARLRKLRKKEAKQRWDDRHWSQKKLDE | |
| | | | | | MTDRDWRIFREDYSITTKGGKIPNPIRSWKDSSLP | |
| | | | | | PHILEVIDKCGYKEPTPIQRQAIPIGLQNRDIIGVAE | |
| | | | | | TGSGKTAAFLIPLLVWITTLPKIDRIEESDQGPYAII | |
| | | | | | LAPTRELAQQIEEETIKFGKPLGIRTVAVIGGISRE | |
| | | | | | DQGFRLRMGCEIVIATPGRLIDVLENRYLVLSRCT | |
| | | | | | YVVLDEADRMIDMGFEPDVQKILEHMPVSNQKPD | |
| | | | | | TDEAEDPEKMLANFESGKHKYRQTVMFTATMPP | |
| | | | | | AVERLARSYLRRPAVVYIGSAGKPHERVEQKVFL | |
| | | | | | MSESEKRKKLLAILEQGFDPPIIIFVNQKKGCDVLA | |
| | | | | | KSLEKMGYNACTLHGGKGQEQREFALSNLKAGA | |
| | | | | | KDILVATDVAGRGIDIQDVSMVVNYDMAKNIEDYI | |
| | | | | | HRIGRTGRAGKSGVAITFLTKEDSAVFYELKQAIL | |
| | | | | | ESPVSSCPPELANHPDAQHKPGTILTKKRREETIF | |
| | | | | | A | |
| 559 | JHU1 | NIV | 3.84581429 | TNFRS | MSPILGYWKIKGLVQPTRLLLEYLEEKYEEHLYER | Full Literal Sequence- |
| | 9343 | OIPI | 8 | F25 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMEQRPRGCAAVAAA | |
| | | | | | LLLVLLGARAQGGTRSPRCDCAGDFHKKIGLFCC | |
| | | | | | RGCPADEAGMEALTPPPATHLSPLDSAHTLLAPP | |
| | | | | | DSSEKICTVQLVGNSWTPGYPETQEALCPQVTW | |
| | | | | | SWDQLPSRALGPAAAPTLSPESPAGSPAMMLQP | |
| | | | | | GPQLYDVMDAVPARRWKEFVRTLGLREAEIEAVE | |
| | | | | | VEIGRFRDQQYEMLKRWRQQQPAGLGAVYAALE | |
| | | | | | RMGLDGCVEDLRSRLQRGP | |
| 560 | JHU1 | NIV | 3.84581429 | TNFRS | MEQRPRGCAAVAAALLLVLLGARAQGGTRSPRC | Core Human ORF Only |
| | 9343 | OIPI | 8 | F25 | DCAGDFHKKIGLFCCRGCPADEAGMEALTPPPAT | |
| | | _TO | | | HLSPLDSAHTLLAPPDSSEKICTVQLVGNSWTPG | |
| | | X | | | YPETQEALCPQVTWSWDQLPSRALGPAAAPTLS | |
| | | | | | PESPAGSPAMMLQPGPQLYDVMDAVPARRWKE | |
| | | | | | FVRTLGLREAEIEAVEVEIGRFRDQQYEMLKRWR | |
| | | | | | QQQPAGLGAVYAALERMGLDGCVEDLRSRLQR | |
| | | | | | GP | |
| 561 | JHU1 | NIV | 1.39655325 | Q640n4 | MSPILGYWKIKGLVQPTRLLLEYLEEKYEEHLYER | Full Literal Sequence- |
| | 9781 | OIPI | 7 | | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMNKSQEQVSFKDVC | |
| | | | | | VDFTKEEWYLLDPAQKVLYRDVILENYSNFVSVG | |
| | | | | | HCVTKPDVIFKIEQGEDPWIPENGFQCHPERKWK | |
| | | | | | VDDLLESNQENQDGHFWPLMFTNKITNLEGGGN | |
| | | | | | TRETPSPDIDSVSSRSFLLKVCDSCEMNLKNVSA | |
| | | | | | VIISKKNYSRKKLNEFNIHKKPLPDSRREKIPVGIK | |
| | | | | | SCDYDGERDGVRLCRDLARPVSHQPFEYNETGQ | |
| | | | | | GFCEEVALCTSQNSQLGETLYKHTECGRTFVDSL | |
| | | | | | KLSVSQKTRLHTELHECSICRKSFYVDLQFGHHT | |
| | | | | | ALSGSSPYERNEYEKLFCDGTAFIVHQEAYTRKIA | |
| | | | | | QEYKVSNRTWGKPALFKHQIIQMGGKPYEYYEN | |
| | | | | | RKNFSKKSHPTQPRRPHVGEKTFECGECGKMF | |
| | | | | | WEKSNLTQHQRTHTGEKPYECTECGKAFCQKPH | |
| | | | | | LTNHQRTHTGEKPYECKQCGKTFCVKSNLTEHQ | |
| | | | | | RTHTGEKPYECNACRKSFCHRSALTVHQRTHTG | |
| | | | | | EKPFICSECGKSFCVKSNLIVHQRTHTGEKPYKC | |
| | | | | | NECGKTFCEKSALTKHQRTHTGEKPYECHVCGK | |
| | | | | | TFSQRSVLTKHLRIHTRVKALLASNPAFLYKVVKA | |
| | | | | | SS | |
| 562 | JHU1 | NIV | 1.39655325 | Q640n4 | MNKSQEQVSFKDVCVDFTKEEWYLLDPAQKVLY | Core Human ORF Only |
| | 9781 | OIPI | 7 | | RDVILENYSNFVSVGHCVTKPDVIFKIEQGEDPWI | |
| | | _TO | | | PENGFQCHPERKWKVDDLLESNQENQDGHFWP | |
| | | X | | | LMFTNKITNLEGGGNTRETPSPDIDSVSSRSFLLK | |
| | | | | | VCDSCEMNLKNVSAVIISKKNYSRKKLNEFNIHKK | |
| | | | | | PLPDSRREKIPVGIKSCDYDGERDGVRLCRDLAR | |
| | | | | | PVSHQPFEYNETGQGFCEEVALCTSQNSQLGET | |
| | | | | | LYKHTECGRTFVDSLKLSVSQKTRLHTELHECSIC | |
| | | | | | RKSFYVDLQFGHHTALSGSSPYERNEYEKLFCD | |
| | | | | | GTAFIVHQEAYTRKIAQEYKVSNRTWGKPALFKH | |
| | | | | | QIIQMGGKPYEYYENRKNFSKKSHPTQPRRPHV | |
| | | | | | GEKTFECGECGKMFWEKSNLTQHQRTHTGEKP | |
| | | | | | YECTECGKAFCQKPHLTNHQRTHTGEKPYECKQ | |
| | | | | | CGKTFCVKSNLTEHQRTHTGEKPYECNACRKSF | |
| | | | | | CHRSALTVHQRTHTGEKPFICSECGKSFCVKSNLI | |
| | | | | | VHQRTHTGEKPYKCNECGKTFCEKSALTKHQRT | |
| | | | | | HTGEKPYECHVCGKTFSQRSVLTKHLRIHTRVKA | |
| | | | | | LLAS | |
| 563 | JHU2 | NIV | 1.94049612 | ALDH1 | MSPILGYWKIKGLVQPTRLLLEYLEEKYEEHLYER | Full Literal Sequence- |
| | 5420 | OIPI | 6 | L2 | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMLRRGSQALRRFST | |
| | | | | | GRVYFKNKLKLALIGQSLFGQEVYSHLRKEGHRV | |
| | | | | | VGVFTVPDKDGKADPLALAAEKDGTPVFKLPKW | |
| | | | | | RVKGKTIKEVAEAYRSVGAELNVLPFCTQFIPMDII | |
| | | | | | DSPKHGSIIYHPSILPRHRGASAINWTLIMGDKKA | |
| | | | | | GFSVFWADDGLDTGPILLQRSCDVEPNDTVDALY | |
| | | | | | NRFLFPEGIKAMVEAVQLIADGKAPRIPQPEEGAT | |
| | | | | | YEGIQKKENAEISWDQSAEVLHNWIRGHDKVPGA | |
| | | | | | WTEINGQMVTFYGSTLLNSSVPPGEPLEIKGAKK | |
| | | | | | PGLVTKNGLVLFGNDGKALTVRNLQFEDGKMIPA | |
| | | | | | SQYFSTGETSVVELTAEEVKVAETIKVIWAGILSN | |
| | | | | | VPIIEDSTDFFKSGASSMDVARLVEEIRQKCGGLQ | |
| | | | | | LQNEDVYMATKFEGFIQKVVRKLRGEDQEVELVV | |
| | | | | | DYISKEVNEIMVKMPYQCFINGQFTDADDGKTYD | |
| | | | | | TINPTDGSTICKVSYASLADVDKAVAAAKDAFENG | |
| | | | | | EWGRMNARERGRLMYRLADLLEENQEELATIEAL | |
| | | | | | DSGAVYTLALKTHIGMSVQTFRYFAGWCDKIQGS | |
| | | | | | TIPINQARPNRNLTFTKKEPLGVCAIIIPWNYPLMM | |
| | | | | | LAWKSAACLAAGNTLVLKPAQVTPLTALKFAELSV | |
| | | | | | KAGFPKGVINIIPGSGGIAGQRLSEHPDIRKLGFTG | |
| | | | | | STPIGKQIMKSCAVSNLKKVSLELGGKSPLIIFNDC | |
| | | | | | ELDKTVRMGMGAVFFNKGENCIAAGRLFVEESIH | |
| | | | | | DEFVTRWEEIKKMKIGDPLDRSTDHGPQNHKAH | |
| | | | | | LEKLLQYCETGVKEGATLVYGGRQVQRPGFFME | |
| | | | | | PTVFTDVEDYMYLAKEESFGPIMVISKFQNGDIDG | |
| | | | | | VLQRANSTEYGLASGVFTRDINKAMYVSEKLEAG | |
| | | | | | TVFINTYNKTDVAAPFGGVKQSGFGKDLGEEALN | |
| | | | | | EYLKTKTVTLEYNPAFLYKVVKASS | |
| 564 | JHU2 | NIV | 1.94049612 | ALDH1 | MLRRGSQALRRFSTGRVYFKNKLKLALIGQSLFG | Core Human ORF Only |
| | 5420 | OIPI | 6 | L2 | QEVYSHLRKEGHRVVGVFTVPDKDGKADPLALA | |
| | | _TO | | | AEKDGTPVFKLPKWRVKGKTIKEVAEAYRSVGAE | |
| | | X | | | LNVLPFCTQFIPMDIIDSPKHGSIIYHPSILPRHRGA | |
| | | | | | SAINWTLIMGDKKAGFSVFWADDGLDTGPILLQR | |
| | | | | | SCDVEPNDTVDALYNRFLFPEGIKAMVEAVQLIAD | |
| | | | | | GKAPRIPQPEEGATYEGIQKKENAEISWDQSAEV | |
| | | | | | LHNWIRGHDKVPGAWTEINGQMVTFYGSTLLNS | |
| | | | | | SVPPGEPLEIKGAKKPGLVTKNGLVLFGNDGKAL | |
| | | | | | TVRNLQFEDGKMIPASQYFSTGETSVVELTAEEV | |
| | | | | | KVAETIKVIWAGILSNVPIIEDSTDFFKSGASSMDV | |
| | | | | | ARLVEEIRQKCGGLQLQNEDVYMATKFEGFIQKV | |
| | | | | | VRKLRGEDQEVELVVDYISKEVNEIMVKMPYQCFI | |
| | | | | | NGQFTDADDGKTYDTINPTDGSTICKVSYASLAD | |
| | | | | | VDKAVAAAKDAFENGEWGRMNARERGRLMYRL | |
| | | | | | ADLLEENQEELATIEALDSGAVYTLALKTHIGMSV | |
| | | | | | QTFRYFAGWCDKIQGSTIPINQARPNRNLTFTKKE | |
| | | | | | PLGVCAIIIPWNYPLMMLAWKSAACLAAGNTLVLK | |
| | | | | | PAQVTPLTALKFAELSVKAGFPKGVINIIPGSGGIA | |
| | | | | | GQRLSEHPDIRKLGFTGSTPIGKQIMKSCAVSNLK | |
| | | | | | KVSLELGGKSPLIIFNDCELDKTVRMGMGAVFFN | |
| | | | | | KGENCIAAGRLFVEESIHDEFVTRVVEEIKKMKIG | |
| | | | | | DPLDRSTDHGPQNHKAHLEKLLQYCETGVKEGA | |
| | | | | | TLVYGGRQVQRPGFFMEPTVFTDVEDYMYLAKE | |
| | | | | | ESFGPIMVISKFQNGDIDGVLQRANSTEYGLASG | |
| | | | | | VFTRDINKAMYVSEKLEAGTVFINTYNKTDVAAPF | |
| | | | | | GGVKQSGFGKDLGEEALNEYLKTKTVTLEY | |
| 565 | JHU2 | NIV | 1.04758712 | PPP2R | MSPILGYWKIKGLVQPTRLLLEYLEEKYEEHLYER | Full Literal Sequence- |
| | 6688 | OIPI | 5 | 2D | DEGDKWRNKKFELGLEFPNLPYYIDGDVKLTQSM | (including GST-tag, 6- |
| | | _TO | | | AIIRYIADKHNMLGGCPKERAEISMLEGAVLDIRYG | His leader, cloning |
| | | X | | | VSRIAYSKDFETLKVDFLSKLPEMLKMFEDRLCHK | adapters, core human |
| | | | | | TYLDGDHVTHPDFMLYDALDVVLYMDPMCLDAF | ORF, occasional ‘post |
| | | | | | PKLVCFKKRIEAIPQIDKYLKSSKYIAWPLQGWQA | stop’ end sequence) |
| | | | | | TFGGGDHPPKSDLVPRGSPGISGGGGGILGRGS | |
| | | | | | HHHHHHGGGITSLYKKAGTMAGAGGGGCPAGG | |
| | | | | | NDFQWCFSQVKGAIDEDVAEADIISTVEFNYSGD | |
| | | | | | LLATGDKGGRVVIFQREQENKSRPHSRGEYNVY | |
| | | | | | STFQSHEPEFDYLKSLEIEEKINKIRWLPQQNAAH | |
| | | | | | FLLSTNDKTIKLWKISERDKRAEGYNLKDEDGRLR | |
| | | | | | DPFRITALRVPILKPMDLMVEASPRRIFANAHTYHI | |
| | | | | | NSISVNSDHETYLSADDLRINLWHLEITDRSFNIVD | |
| | | | | | IKPANMEELTEVITAAEFHPHQCNVFVYSSSKGTI | |
| | | | | | RLCDMRSSALCDRHSKFFEEPEDPSSRSFFSEIIS | |
| | | | | | SISDVKFSHSGRYMMTRDYLSVKVWDLNMESRP | |
| | | | | | VETHQVHEYLRSKLCSLYENDCIFDKFECCWNGS | |
| | | | | | DSAIMTGSYNNFFRMFDRDTRRDVTLEASRESSK | |
| | | | | | PRASLKPRKVCTGGKRRKDEISVDSLDFNKKILHT | |
| | | | | | AWHPVDNVIAVAATNNLYIFQDKINNPAFLYKVVK | |
| | | | | | ASS | |
| 566 | JHU2 | NIV | 1.04758712 | PPP2R | MAGAGGGGCPAGGNDFQWCFSQVKGAIDEDVA | Core Human ORF Only |
| | 6688 | OIPI | 5 | 2D | EADIISTVEFNYSGDLLATGDKGGRVVIFQREQEN | |
| | | _TO | | | KSRPHSRGEYNVYSTFQSHEPEFDYLKSLEIEEKI | |
| | | X | | | NKIRWLPQQNAAHFLLSTNDKTIKLWKISERDKRA | |
| | | | | | EGYNLKDEDGRLRDPFRITALRVPILKPMDLMVEA | |
| | | | | | SPRRIFANAHTYHINSISVNSDHETYLSADDLRINL | |
| | | | | | WHLEITDRSFNIVDIKPANMEELTEVITAAEFHPHQ | |
| | | | | | CNVFVYSSSKGTIRLCDMRSSALCDRHSKFFEEP | |
| | | | | | EDPSSRSFFSEIISSISDVKFSHSGRYMMTRDYLS | |
| | | | | | VKVWDLNMESRPVETHQVHEYLRSKLCSLYEND | |
| | | | | | CIFDKFECCWNGSDSAIMTGSYNNFFRMFDRDT | |
| | | | | | RRDVTLEASRESSKPRASLKPRKVCTGGKRRKD | |
| | | | | | EISVDSLDFNKKILHTAWHPVDNVIAVAATNNLYIF | |
| | | | | | QDKIN | |

In embodiments, the amount of antibodies bound to a proteome array is scored, for example according to the Common Terminology Criteria for Adverse Events (CTCAE). Samples may be divided into groups as further set forth herein. Thus, the disclosure provides compositions and methods for antibody profiling. The antibody profiling may be carried out prior to treatment, any time during the treatment or any time after the treatment. The profiling may be carried out once or multiple times over any period of time.

In one aspect, the present disclosure provides methods for enhancing the efficacy of treatment of cancer, such as melanoma, with immune checkpoint inhibitors. The disclosure also provides panels for detection of subsets of antibodies that can form a basis for treatment decisions in the treatment of cancer, such as melanoma. The disclosure also provides kits for detection of specific antibodies.

In embodiments, a method of this disclosure comprises: a) obtaining a sample of a biological sample, such as blood, plasma or serum, b) determining antibodies using a protein array; and c) based on the profile of the antibodies, determining that the individual is not a candidate for a checkpoint inhibitor, or administering one or more immune checkpoint inhibitors to the individual. The method can further comprise administering to the individual agents to mitigate expected or observed toxicity from the checkpoint inhibitors.

The amount of antibodies, or a change in the level of antibodies, means a level that is measured against a suitable reference, such as a reference value. The reference may be established from a population of relevant individuals from which group the distinction is to be made. For example, the reference can be an average value from a group of individuals who have not shown toxicity, shown mild toxicity, or shown severe toxicity to the particular treatment. These values could be used as references for no toxicity, mild toxicity or severe toxicity, or site-specific toxicity. Other references can be obtained in a similar manner. For no toxicity, individuals who have not been treated at all may also be used.

The presence, absence and amount of antibodies in a patient sample can be detected by methods that are known in the art. For example, any type of immunological assay or antigen binding assay may be used. A commonly used assay is ELISA. Detection of the antigen-antibody complex is generally done by using detectable (fluorescent, luminescent, chemiluminescent, radioactive etc.) labels.

In one embodiment, a patient who is predicted to develop severe toxicity (or severe toxicity affecting specific organ/tissue sites or likely requiring treatment termination) could be monitored for the development of toxicity, or could be treated with a different dosage of immune checkpoint inhibitor(s). Such monitoring could allow clinicians to intervene e.g. with steroids, to mitigate toxicity (immune related adverse events) as they develop.

In one embodiment, the present methods can also be used in adjuvant immune checkpoint blockade in earlier stage (3 or 2) melanoma as being able to identify patients at risk of severe toxicity would be especially beneficial in the adjuvant setting, where there is less tolerance for severe toxicity. If an indication of likelihood of toxicity is observed, then steps can be taken to mitigate the toxicity, or the treatment regimen of anti-CTLA-4 and/or anti-PD-1 can be interrupted or the dose reduced. For mitigating toxicity, corticosteroid treatment may be administered. For example, prednisone may be administered orally or via i.v. For skin rashes, topical corticosteroids may be used. Another approach is to administer a tumor necrosis factor-alpha (TNF-α) inhibitor prior to or concurrent with one or a combination of immune checkpoint inhibitors. A non-limiting embodiment of a suitable TNF-α inhibitor is infliximab, but other TNF-α inhibitors may also be used. Non-limiting examples of other suitable TNF-α inhibitors include Infliximab-abda, Infliximab-dyyb. Adalimumab, Adalimumab-adaz, Adalimumab-atto, Certolizumab pegol, Etanercept, Etanercept-SZZS, and Golimumab. Other treatments for steroid-refractory irAEs—typically colitis—include: mycophenolic acid, or tacrolimus.

When recurrence of cancer is predicted, another anti-cancer agent can be used, e.g., any other anti-cancer agent that is not the immune checkpoint inhibitor or combination of immune checkpoint inhibitors for which recurrence is predicted after treatment. Alternatively dosing of the one or more checkpoint inhibitors can be change. In embodiments, a different checkpoint inhibitor or combination of checkpoint inhibitors is used.

With respect to the foregoing description, given the overlap in the clinical presentation of irAEs and conventional autoimmune disorders, the disclosure relates to the discovery that certain individuals possess a subclinical predisposition for ICB toxicity, which is characterized by the presence of preexisting autoantibodies (autoAbs) and does not necessarily manifest spontaneously but can be unmasked following checkpoint blockade. Without intending to be bound by any particular theory, it is considered that prior to the present disclosure, there has been no clinically validated and accurate tool for predicting immunotherapy efficacy and/or immune-related toxicity in melanoma patients.

The following Examples are intended to illustrate but not limit the disclosure.

### EXAMPLES

Methods

Patient Population

This disclosure included 950 patients from two Phase 3, randomized, double-blind trials of adjuvant immunotherapy in resected melanoma. The first dataset was composed of 565 patients who were enrolled in CheckMate-238 (NCT02388906), which was an investigation of adjuvant nivolumab versus ipilimumab in patients with high risk, completely resected stage IIIB, IIIC, or IV melanoma, as defined by the American Joint Committee on Cancer (AJCC) 7^{th} Edition. The cohort was retrospectively chosen by the trial sponsor (Bristol Myers Squibb), was enriched for immune-related adverse events, and included 408 patients who received ipilimumab and 157 patients treated with nivolumab.

The second dataset consisted of 385 patients who were enrolled in CheckMate-915 (NCT03068455), which evaluated adjuvant nivolumab plus ipilimumab versus nivolumab monotherapy in patients who underwent complete resection of AJCC 8^{th} Edition stage IIIB, IIIC, IIID, or IV melanoma. The patients were chosen at random by the trial sponsor (Bristol Myers Squibb) who was blinded to their recurrence and toxicity outcomes at the time of selection. In total, there were 190 patients who received nivolumab and 195 patients who received ipilimumab plus nivolumab. For both the CheckMate 238 and CheckMate 915 patients, all clinical and demographic data were obtained directly from the trial sponsor

Clinical Outcomes

The outcomes of interest were: (1) disease recurrence versus no recurrence, and (2) severe (Grade 3-5) irAEs versus no or mild (Grade 1-2) irAEs. Briefly, recurrence-free survival (RFS) was defined as the time from randomization until the date of first recurrence, new primary melanoma, death from any cause, or last follow-up if none of the above occurred. Toxicity events were classified as either “related” or “not related” to the study drug and events categorized as “not related” were excluded from the current disclosure, as were events determined not to be immune-related. Treatment-related events with a potential immunologic etiology were identified using a list of prespecified terms from the Medical Dictionary for Regulatory Activities. For the present disclosure, the events were then categorized based on the body or organ system of origin. The severity of irAEs was graded using the National Cancer Institute (NCI) Common Terminology Criteria for Adverse Events (CTCAE), version 4.0. Grade 3, 4, and 5 irAEs were classified as severe and grade 1 and 2 irAEs were classified as mild.

Serum Preparation and Processing

Peripheral blood samples were prospectively collected within 72-hours of administering the first dose of study medication (day 1 of week 1). The whole blood was allowed to clot at room temperature. The samples were then centrifuged at room temperature at 1100-1300×g for 10 minutes (swing out) or 15 minutes (fixed) until the clot and serum were separated. The serum was next transferred into separate polypropylene tubes and ultimately stored at −70° C.

Serum Autoantibody Profiling

To profile serum autoantibodies, we utilized the HuProt Human Proteome Microarray v4.0 (CDI Laboratories, Mayaguez, PR), which contains over 21,000 unique, individually purified full-length human proteins in duplicate, covering more than 81% of the proteome (Jeong et al., 2012). The HuProt arrays were first blocked with blocking solution (5% BSA/1×TBS-T) at room temperature for 1 hour and then probed with serum samples (diluted 1:1000) at 4° C. overnight. Next, the arrays were washed with 1×TBS-T a total of 3 times (10 minutes per wash). The arrays were then probed with Alexa-647 labeled anti-human IgG Fc gamma fragment specific secondary antibody (Jackson ImmunoResearch, West Grove, Pa.) at room temperature for 1 hour. This was followed by three washes of 1×TBS-T (10 minutes per wash). The arrays were subsequently dried with an air duster and scanned using a GenePix 4000B instrument (Molecular Dynamics, Sunnyvale, Calif.). GenePix Pro (v7.2.30) software was used to measure the signal intensities for IgG binding to array features as well as any background signal present. Array signal intensities data were quality controlled for successful printing, staining, and scanner alignment using internal software tools (CDI Laboratories, Mayaguez, PR), which were used to confirm that duplicate spots retained >0.95 R² signal intensity correlation across each array. The net signal was the background-subtracted median intensity of each antibody spot. This background-subtracted signal intensity was log 2 transformed and normalized to the median of the total signal intensities on the array for each subject. This was done as an internal control to normalize for the various overall levels of autoAb expressions among subjects. We then standardized each autoAb intensity by its mean and standard deviation across all subjects.

Prediction of Disease Recurrence and Severe Toxicity

Patients who received nivolumab from the CheckMate 238 cohort were randomly divided into discovery and test sets in a ratio of 75% to 25%. The nivolumab arm of the CheckMate 915 cohort was used as an independent validation dataset. Patients who received ipilimumab from CheckMate 238 and patients who received ipilimumab plus nivolumab from CheckMate 915 were each randomly split into discovery and test sets in a ratio of 75% to 25%. Descriptive group comparisons were performed using two-sample t-tests and chi-square tests for continuous and categorical variables, respectively.

The signatures for predicting recurrence and severe toxicity were independently derived from the discovery datasets for each treatment regimen but followed a parallel process. We first employed univariable two-sample t-tests to assess the association between every autoAb candidate and the outcome of interest. We categorized autoAbs as differentially expressed autoantibodies (DEA) if they met 2 criteria: (1) they had higher intensities in the no recurrence group compared to the recurrence group or in the severe toxicity compared to the no severe toxicity group, and (2) the univariate P value was less than 0.05. We then performed stability selection in combination with least absolute shrinkage and selection operator (LASSO) regression on the complete set of DEA to identify a parsimonious subset of autoAbs that would constitute the final signature (Tibshirani et al., 1996; Meinshausen et al., 2010). The cut-off value for stable selection was set at 75% (the percentage of times a variable was selected into a model).

The capacity of the autoAb signatures to predict recurrence or severe toxicity in the test and validation datasets was evaluated using area under the curve (AUC) of the receiver operating characteristic (ROC) curve. In addition, we identified a cutoff prediction score from the discovery sets and used it to stratify patients from the test and validation sets into high- versus low-risk groups. We selected cut points that achieved a minimum negative predictive value (NPV) of 80% to assure high accuracies for the patients predicted to have severe toxicity or no recurrence. For the nivolumab cohorts, the same cutoff used for the test set was applied to the independent validation dataset to assess its reproducibility. The sensitivity, specificity, positive predictive value (PPV) and NPV were estimated at this threshold. Recurrence free survival of the predicted high- versus low-likelihood of recurrence groups was compared by Kaplan-Meier curves using the log-rank test. Waterfall plots were generated to illustrate the performance of the toxicity signatures. Finally, the predictive utility of the autoAb signatures was assessed by comparing their AUC, by Delong's Test, to that of a model containing multiple clinical covariates including age, gender, BRAF mutation status, disease stage at study entry, ECOG performance status, baseline LDH (U/L), and PD-L1 status at a cutoff of 5%, which was the threshold used in CheckMate 238 (DeLong et al., 1998). All statistical analyses were performed using R (version 4.1.0, The R Foundation for Statistical Computing).

Functional and Enrichment Analyses of Differentially Expressed Autoantibodies

We started by using Metascape to perform broad-based functional analyses of the differentially expressed autoAb targets (metascape.org). We then performed more targeted enrichment analysis on select pathways from the Kyoto Encyclopedia of Genes and Genomes (KEGG), WikiPathways, and Human Phenotype Ontology (HPO) databases. This included lists of key genes associated with conventional autoimmune disease pathogenesis, cutaneous melanoma, and immunotherapy-related pathways. In addition, we independently compiled a list of proteins reported in conventional autoimmune disorders and a list of known melanoma autoantigens such as members of the Melanoma Antigen Gene (MAGE) and B melanoma antigen (BAGE) families (Hodi, 2006; Tio et al., 2019). The statistical significance of autoAb enrichment was calculated using the hypergeometric test (Federico et al., 2020).

Results

Study Population

Tables 1a-c show that the baseline clinical and demographic characteristics were generally well-balanced between the discovery and test datasets for each of the three regimens. However, as shown in Table 2 and Supplemental Table 1, there were several differences between the patients from CheckMate 238 and CheckMate 915. As a result, there was corresponding discordance between the nivolumab discovery and test sets (from CheckMate 238) and the validation set (from CheckMate 915). For instance, among the former two, more individuals had a baseline ECOG performance status of 1 rather than 0 (10.3% vs. 12.5% vs. 3.7%; P=0.029). Nivolumab patients from the discovery and test sets also had longer RFS than those from the validation set (24.73 vs. 27.53 vs. 21.03 months; P=0.002), but there was no significant difference in the percentage of patients to suffer recurrence (P=0.36). Finally, compared to patients from the validation set, there were more individuals from the discovery and test sets who experienced severe toxicity (39.3% vs. 45.0% vs. 22.1%; P=0.001), which was expected due to the enrichment sampling of the CheckMate 238 analysis cohort.

Prediction of Disease Recurrence and Severe Toxicity

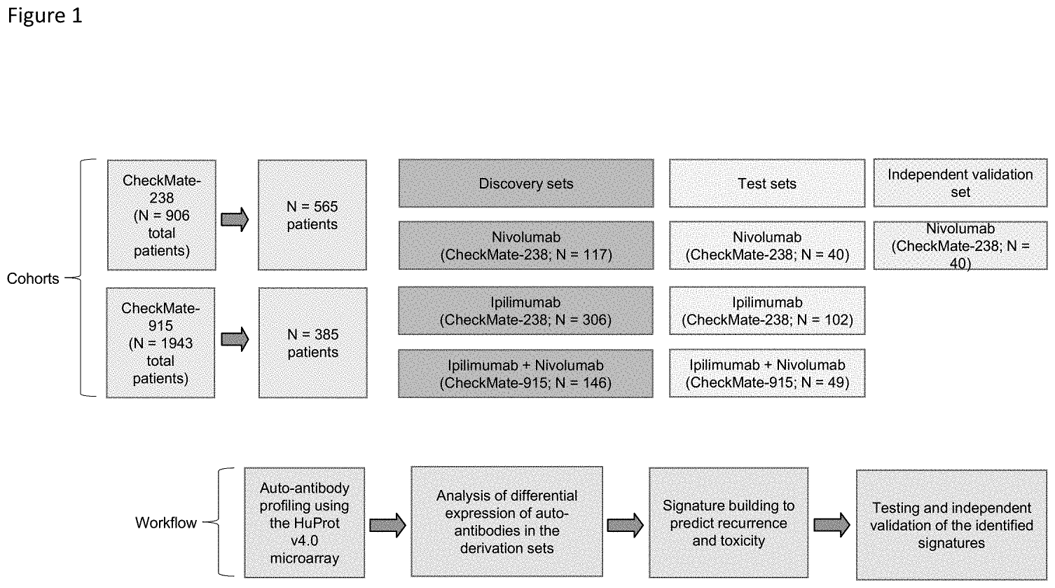

The experimental design and workflow of the experiments are shown in FIG. 1. The final assay panel includes a composite of 297 autoAbs with six subsets that can be used to predict disease recurrence or severe toxicity in response to nivolumab, ipilimumab, or ipilimumab plus nivolumab. The list of autoAbs and their corresponding outcomes of interest is as described herein. The individual predictive performance of each signature is summarized in Table 3 and described in further detail below.

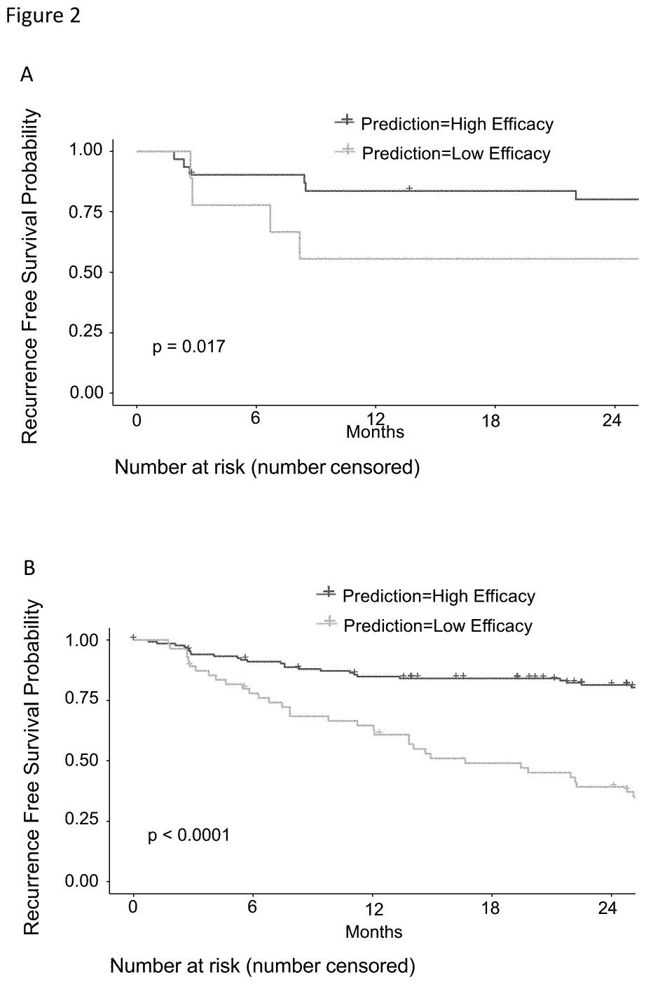

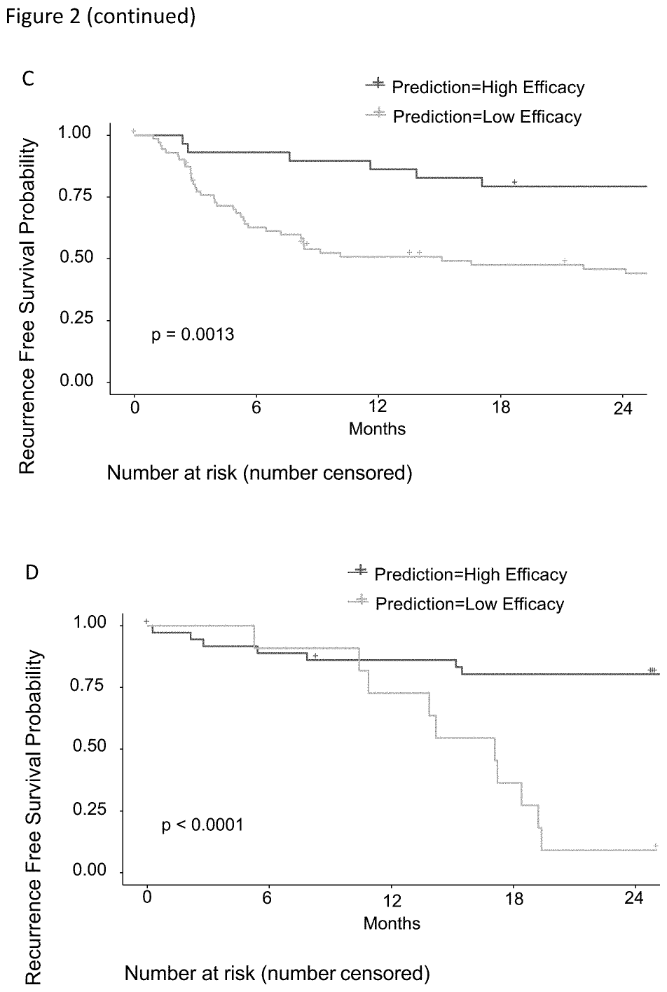

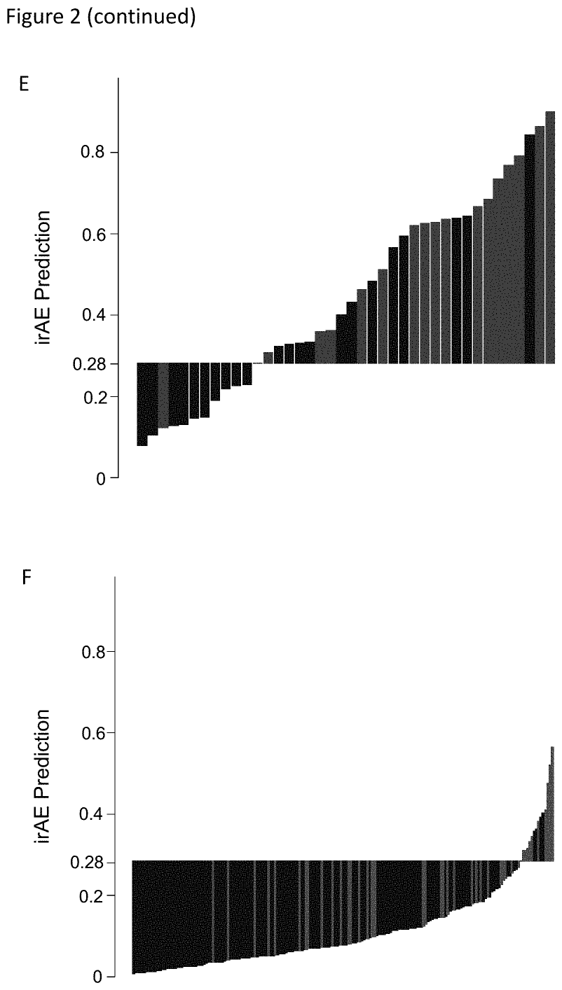

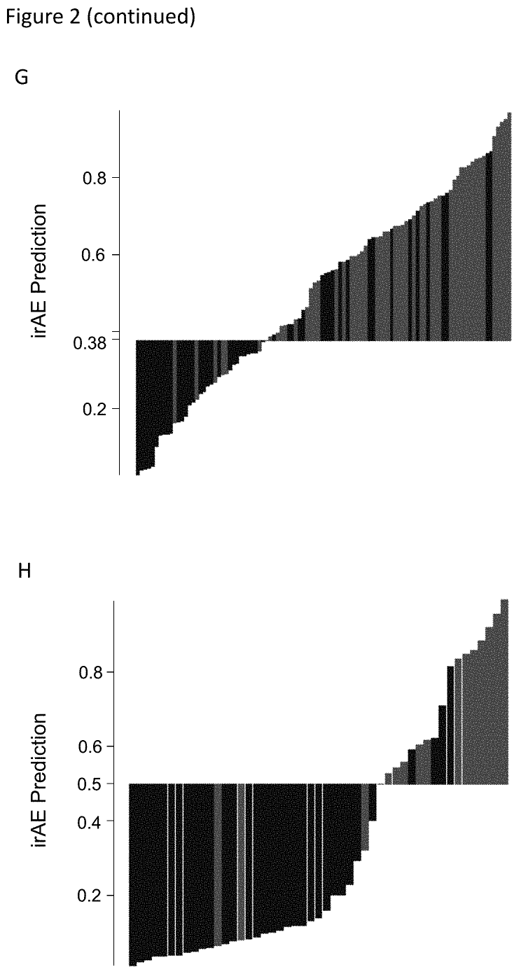

The nivolumab recurrence signature includes a total of 29 autoAbs. It performed with AUC 0.84 (95% CI: 0.71-0.97) and NPV 0.81 (95% CI: 0.53-0.96) on the test set of 40 CheckMate 238 patients. The signature achieved AUC 0.82 (95% CI: 0.75-0.88) and NPV 0.80 (0.70-0.88) when applied to the independent validation dataset of 190 CheckMate 915 patients (Table 3). Kaplan-Meier analyses show that the patients assigned to the high efficacy group had significantly better RFS than patients predicted to experience low efficacy (for the test set, p=0.017; for the validation set, p<0.0001; FIG. 2 panel a b). The nivolumab severe toxicity signature includes 47 autoAbs. It predicted severe toxicity with AUC 0.78 (95% CI: 0.63-0.93) and NPV 0.91 (95% CI: 0.61-0.96) on the test set from CheckMate 238, and AUC 0.75 (0.67-0.83) and NPV 0.82 (95% CI: 0.68-0.94) on the independent validation set from CheckMate 915 (Table 3). A waterfall plot illustrates the relationship between predicted severe toxicity score and the actual development of severe toxicity (FIG. 2 panels e and f).

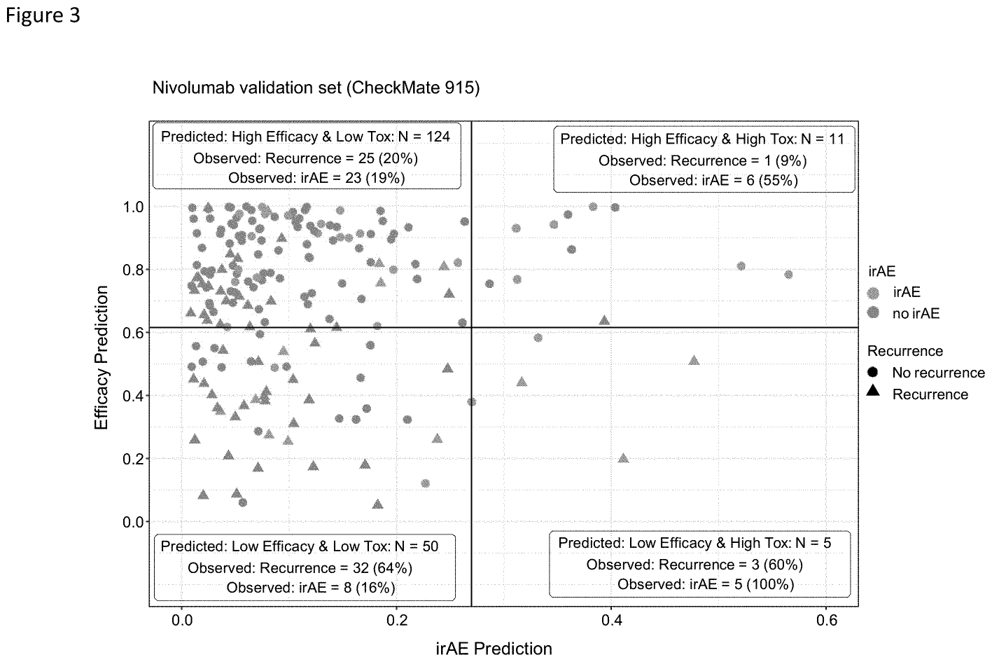

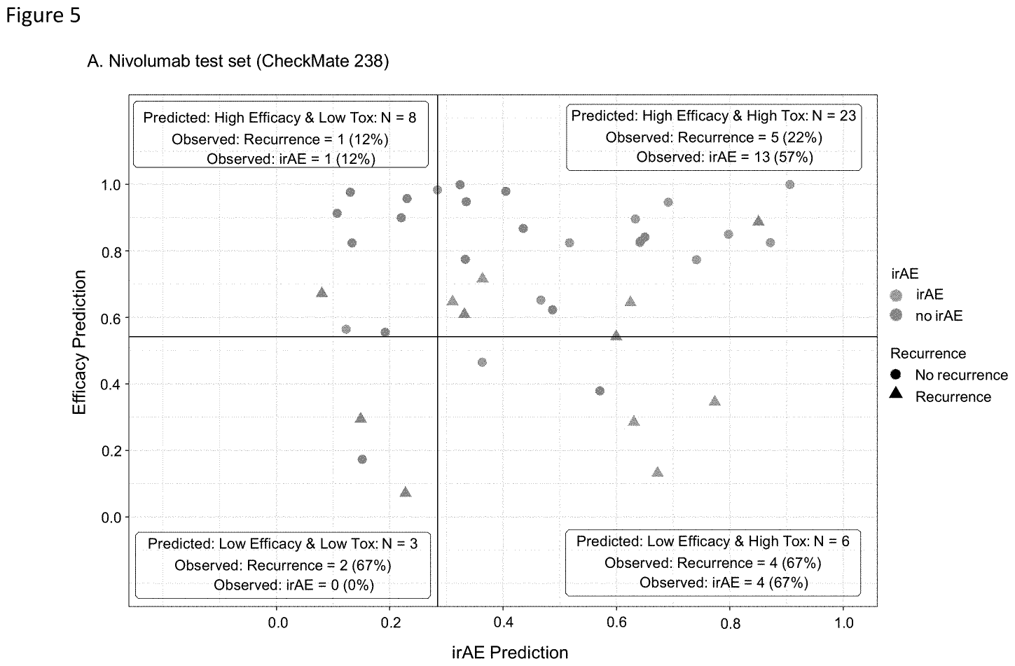

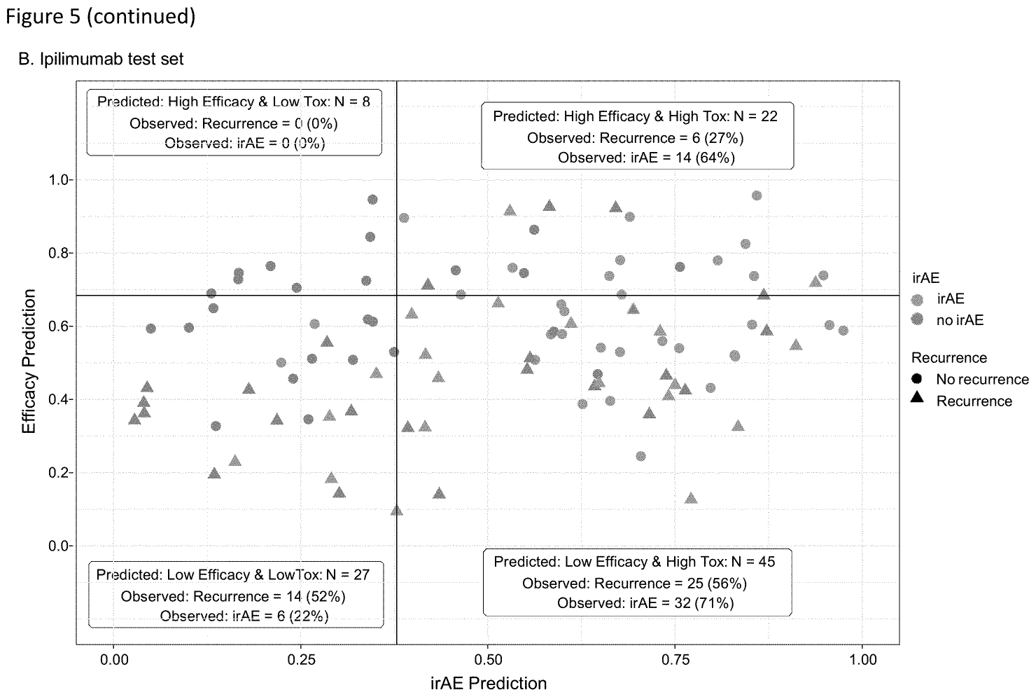

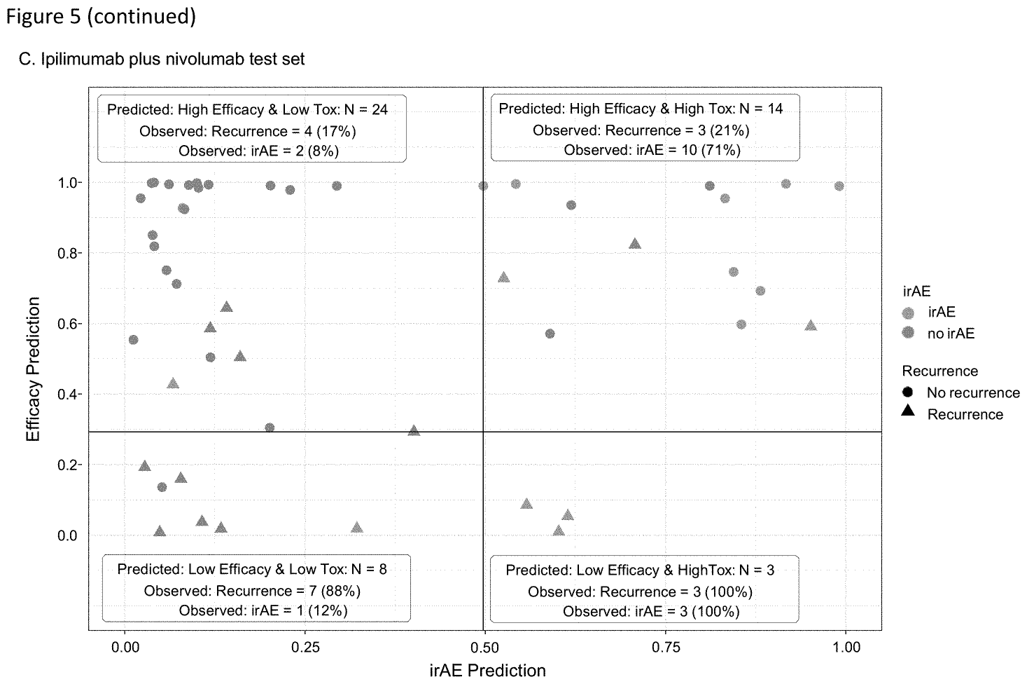

FIG. 3 shows that the nivolumab recurrence and severe toxicity signatures could be used together to accurately stratify patients from the validation dataset into four different combined outcomes quadrants. In the nivolumab validation set, 124 patients were projected to have no recurrence and no severe toxicity; of these, only 25 (20%) recurred and 23 (19%) developed severe toxicity. There were 11 patients predicted to have no recurrence but severe toxicity; of these, only 1 (9%) recurred and 6 (55%) suffered severe toxicity. A total of 50 patients were predicted to suffer disease recurrence but no severe toxicity; 32 (64%) recurred and 8 (16%) had severe toxicity. Only 5 patients were predicted to have disease recurrence and severe toxicity; 3 (60%) recurred and all 5 (100%) had severe toxicity. The results for the nivolumab test set are shown in FIG. 5, panel a.

The ipilimumab recurrence signature consists of 55 autoAb and performed with AUC 0.76 (95% CI: 0.66-0.85) and NPV 0.80 (95% CI: 0.63-0.87) on the test set of 102 CheckMate-238 patients (Table 3). Kaplan-Meier analyses showed that patients predicted to have a low likelihood of recurrence had significantly better RFS (p=0.0013) [FIG. 2 panel c]. The ipilimumab toxicity signature includes 63 autoAbs and achieved AUC 0.79 (95% CI: 0.70-0.88) and NPV 0.83 (95% CI: 0.67-0.90) when applied to the test set (Table 3). A waterfall plot shows the development of severe toxicity according to predicted toxicity score (FIG. 2 panel g). FIG. 1, panel b, shows that the recurrence and toxicity signatures could be used together to accurately stratify patients into four combined outcomes quadrants.

The recurrence signature for ipilimumab plus nivolumab includes 40 autoAbs. It performed with AUC 0.92 (95% CI: 0.85-0.99) and NPV 0.82 (95% CI: 0.60-0.99) on the test set of 49 CheckMate-915 patients (Table 3). Kaplan Meier analyses showed that patients could be stratified into two groups (high versus low risk for recurrence) with significantly different RFS (p<0.0001) [FIG. 2 panel d]. The ipilimumab plus nivolumab toxicity signature includes 39 autoAbs. It achieved AUC 0.87 (95% CI: 0.75-0.99) and NPV 0.91 (95% CI: 0.73-0.97) when applied to the test set (Table 3). A waterfall plot depicts the development of severe toxicity according to predicted toxicity score (FIG. 2 panel h). FIG. 5, panel c, shows that the recurrence and severe toxicity signatures could be used together to accurately stratify patients into four combined outcome quadrants.

Comparison to a Model Composed of Clinical Covariates and Percent PD-L1 Expression

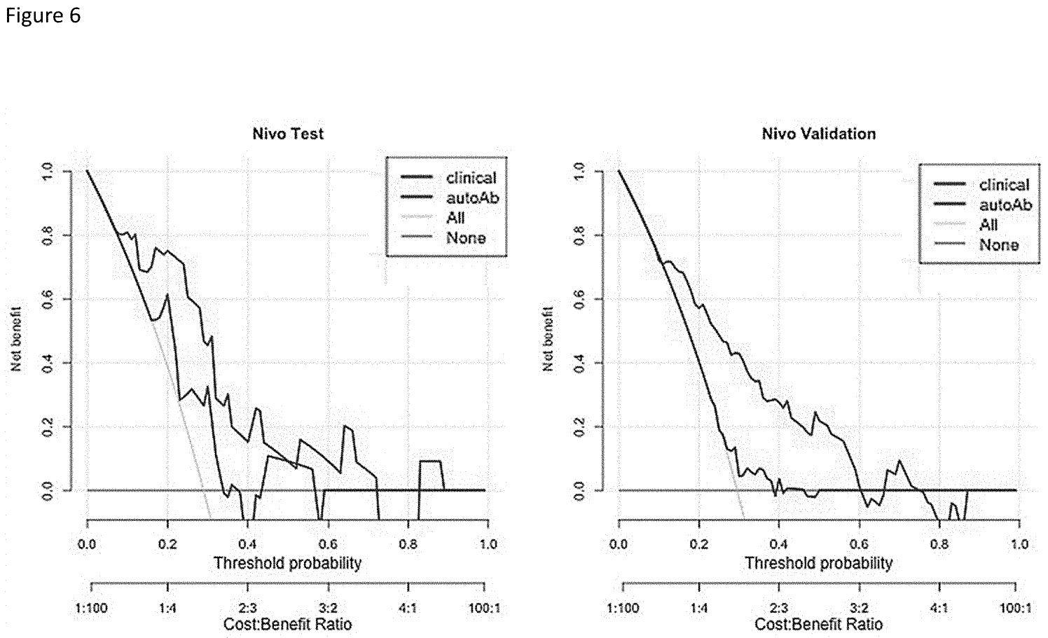

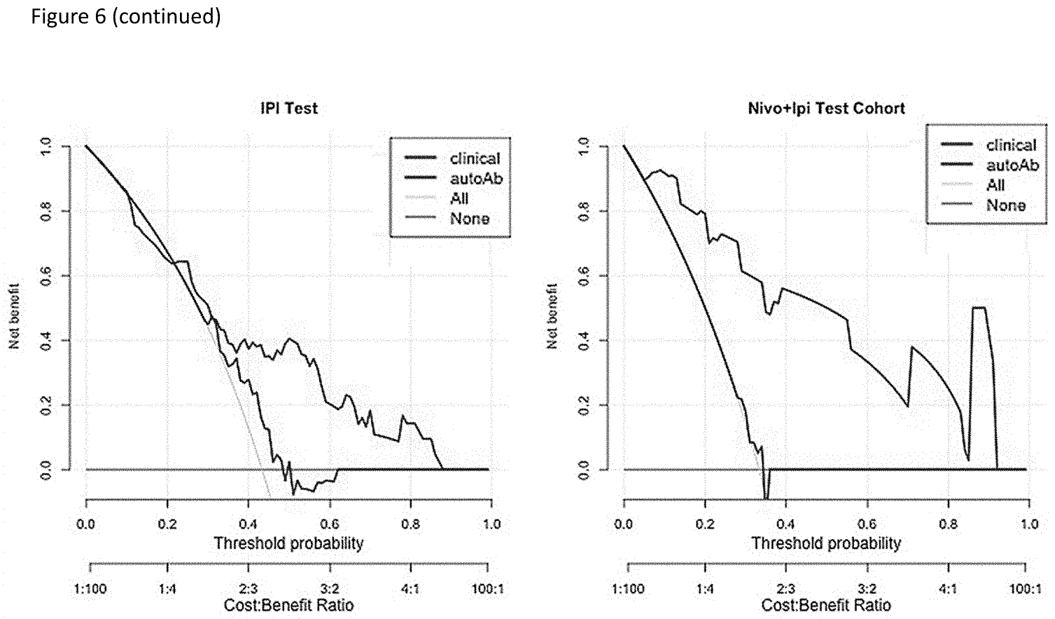

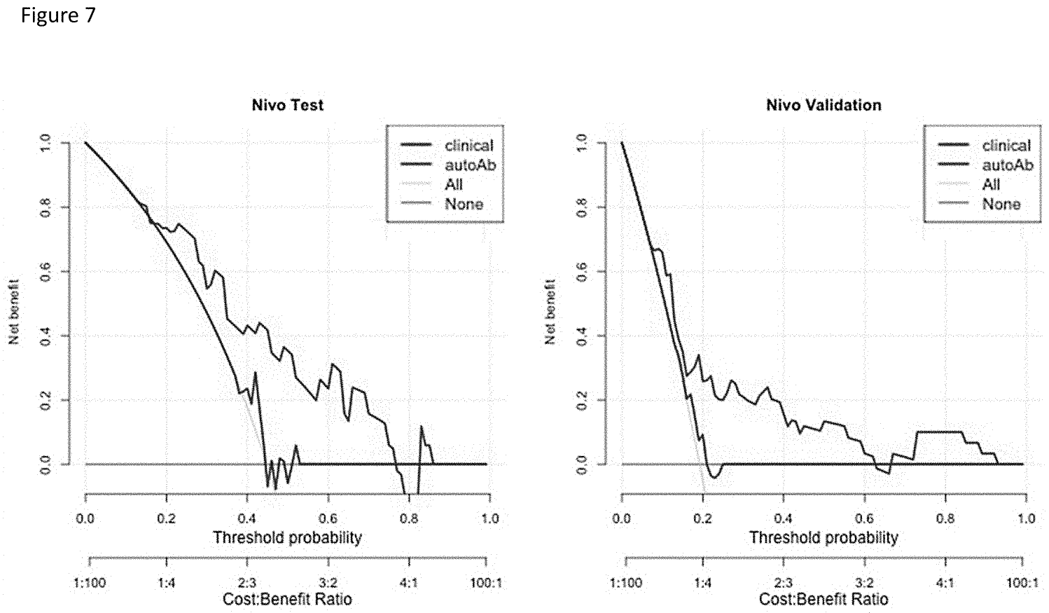

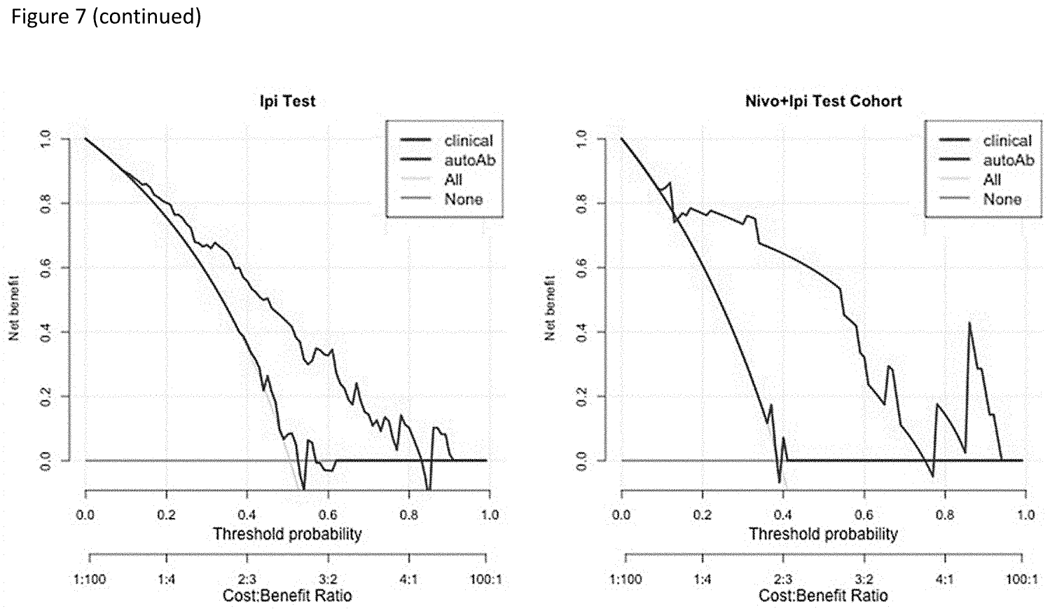

For disease recurrence, the autoAb signatures outperformed the clinical model composed of predictors including age, gender, BRAF mutation status, disease stage at study entry, ECOG performance status, baseline LDH (U/L), and PD-L1 status, on the nivolumab test set (AUC 0.84 vs. 0.66; P=0.124), the nivolumab validation set (AUC 0.82 vs. 0.56; P<0.001), ipilimumab test set (AUC 0.76 vs. 0.59; P=0.028), and ipilimumab plus nivolumab test set (AUC 0.92 vs. 0.51; P<0.0001). For toxicity, the autoAb signatures outperformed the clinical model on the nivolumab test set (AUC 0.78 vs. 0.56; P=0.106), the nivolumab validation set (AUC 0.75 vs. 0.56; P=0.006), ipilimumab test set (AUC 0.79 vs. 0.55; P=0.001), and ipilimumab plus nivolumab test set (AUC 0.87 vs. 0.51; P=0.006, Supplemental Table 2]. Decision curve analyses showed that using the autoAb signatures added net clinical benefit as compared to using the clinical models, or the strategies of treating all patients or no patients (FIGS. 6 and 7).

Functional and Enrichment Analyses of Differentially Expressed AutoAb (DEA)

We approached the analysis of the DEA by investigating: (1) what are the enriched biological and functional roles of the DEA targets, and (2) are the DEA targets also known to be autoantigens in conventional autoimmune diseases, or melanoma neoantigens, or elements of either the PD-1 or CTLA-4 immune checkpoint signaling cascades?

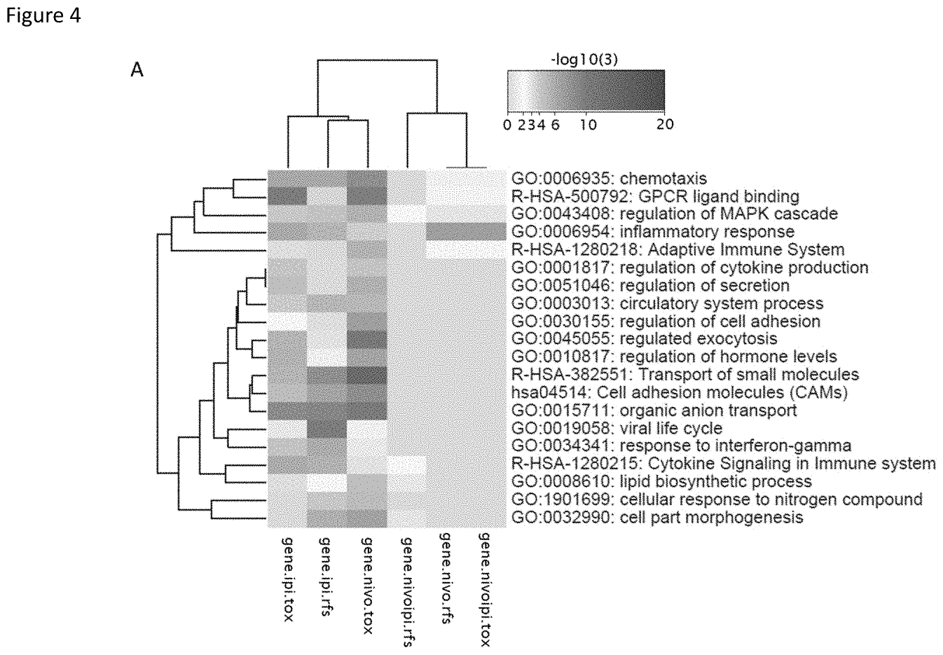

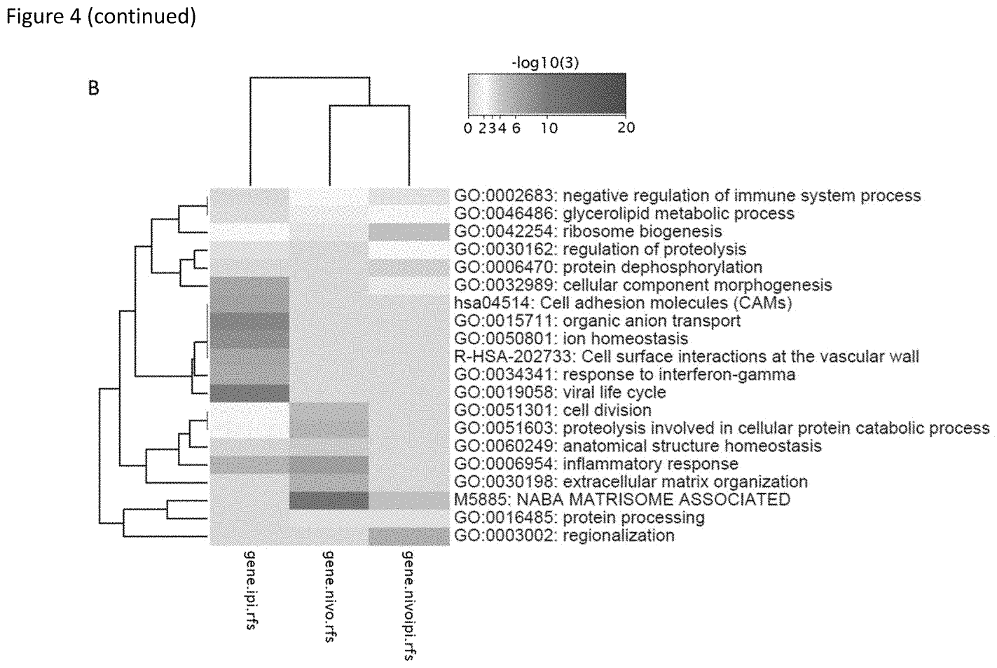

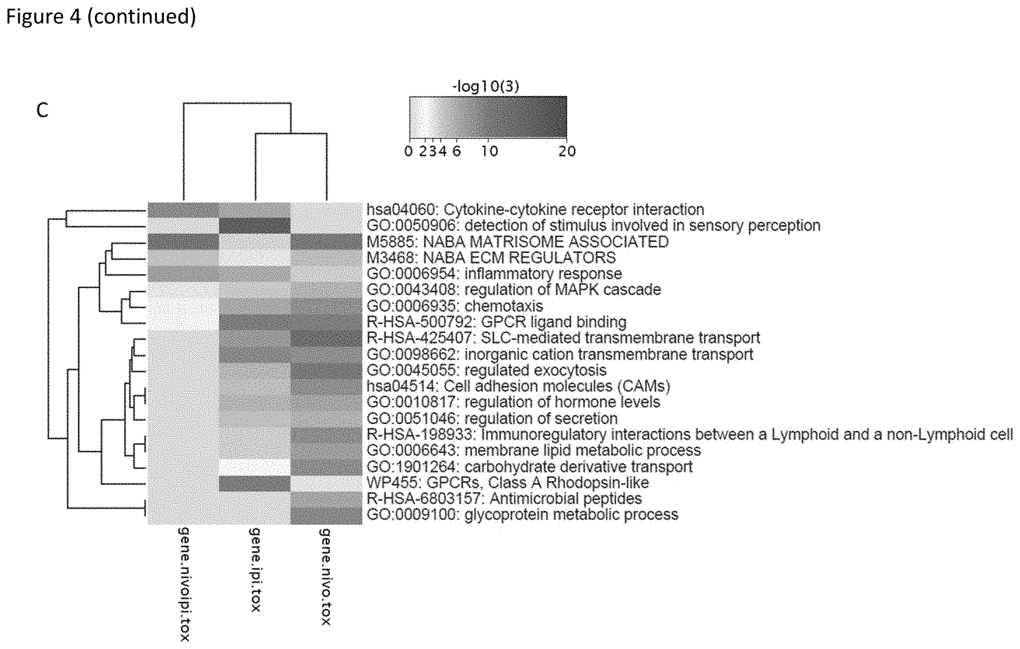

To understand the processes driving baseline humoral immune system activity predictive of ICB response, we first compared the biological and functional roles of the DEA autoantigen targets for the six treatment and outcome combinations (FIG. 4). The analysis of DEA associated with severe toxicity showed overlapping enrichment for antigens involved with the immune related pathways “inflammatory response” and “chemotaxis.” The three sets of autoantigens were also enriched for “GPCR ligand binding” as well as the extracellular pathways “NABA matrisome associated” and “NABA ECM regulators” (FIG. 4 panel c). The DEA profiles associated with disease recurrence were concomitantly enriched for antigens related to the “negative regulation of immune system process”. Both the ipilimumab and nivolumab profiles were enriched for “inflammatory response,” and both nivolumab and nivolumab plus ipilimumab were enriched for “NABA matrisome associated” (FIG. 4b).

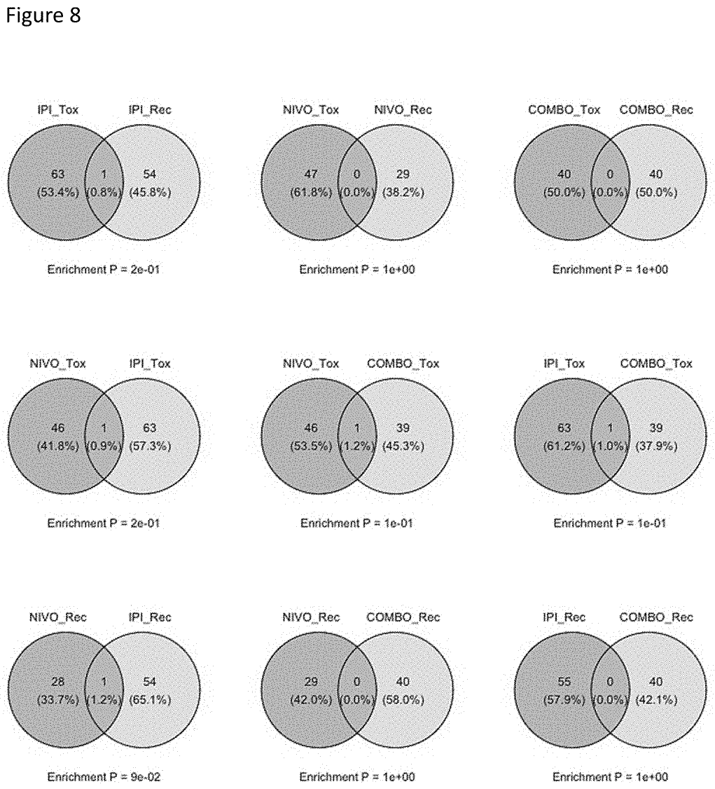

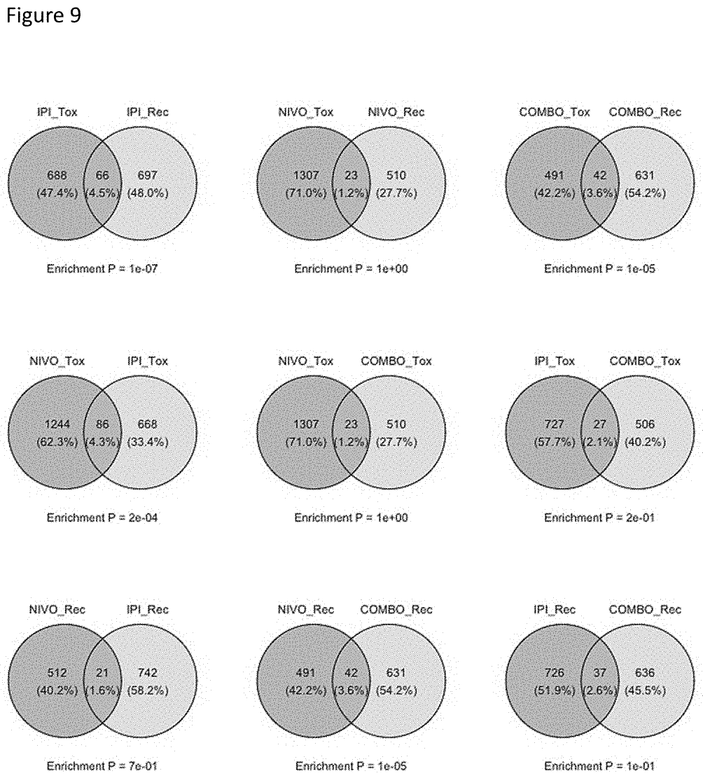

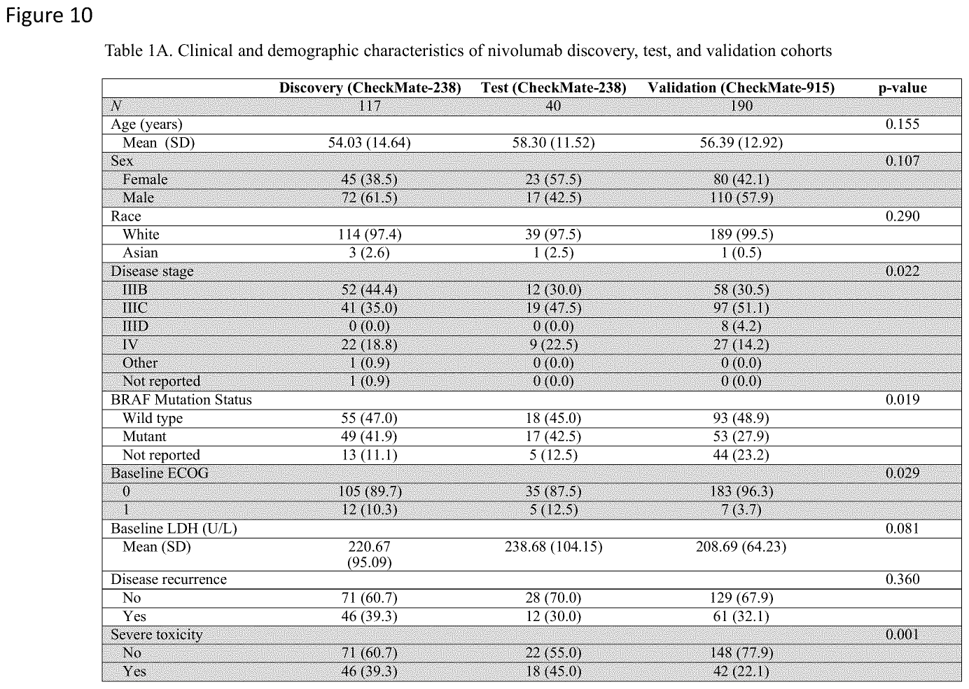

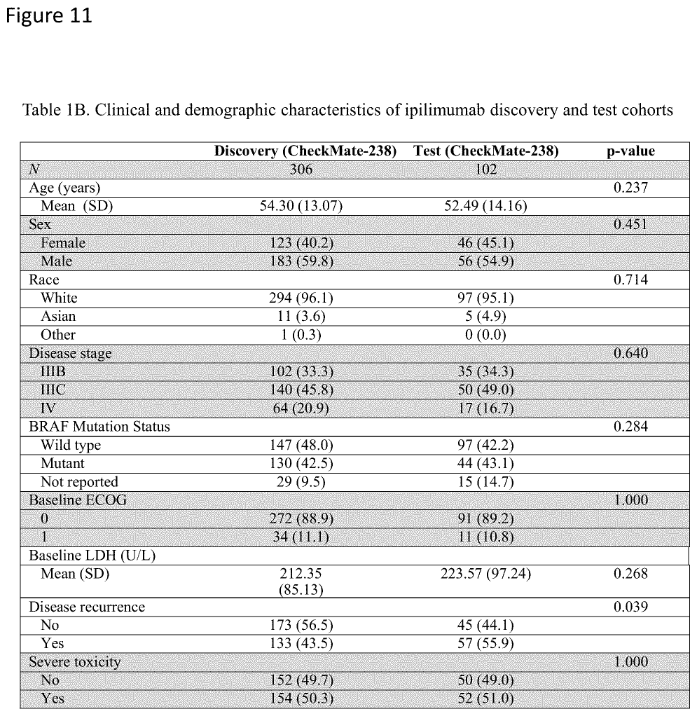

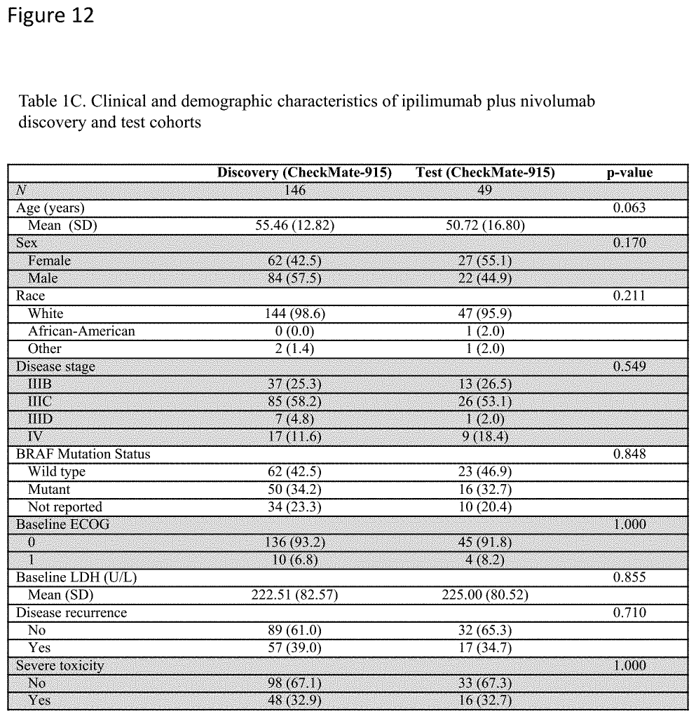

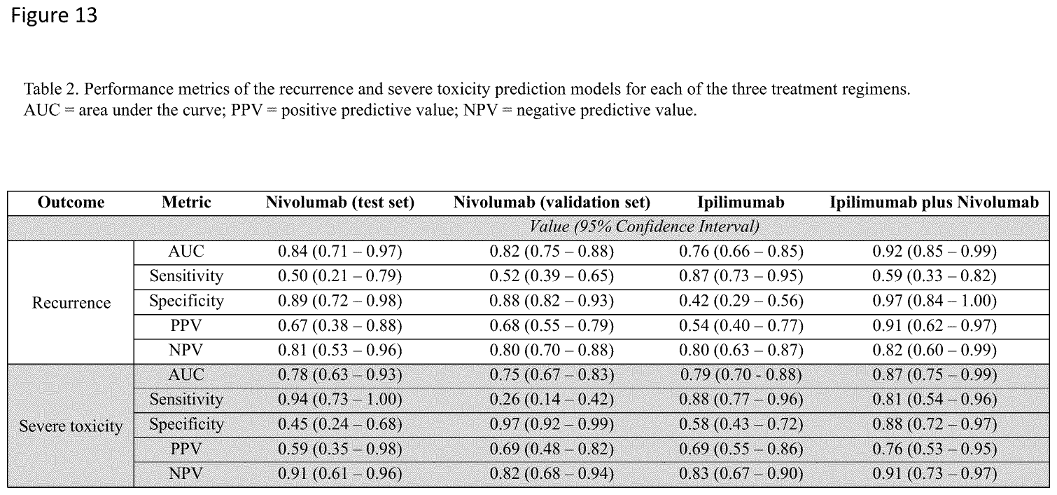

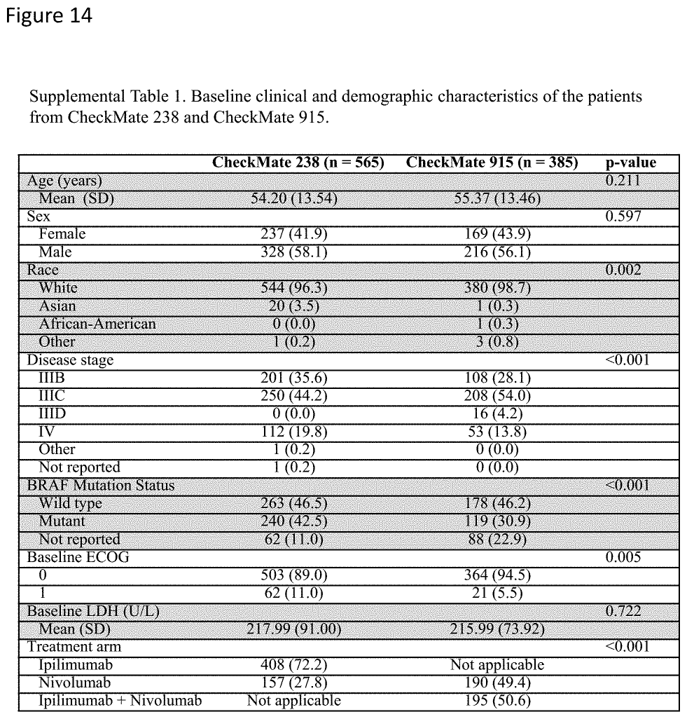

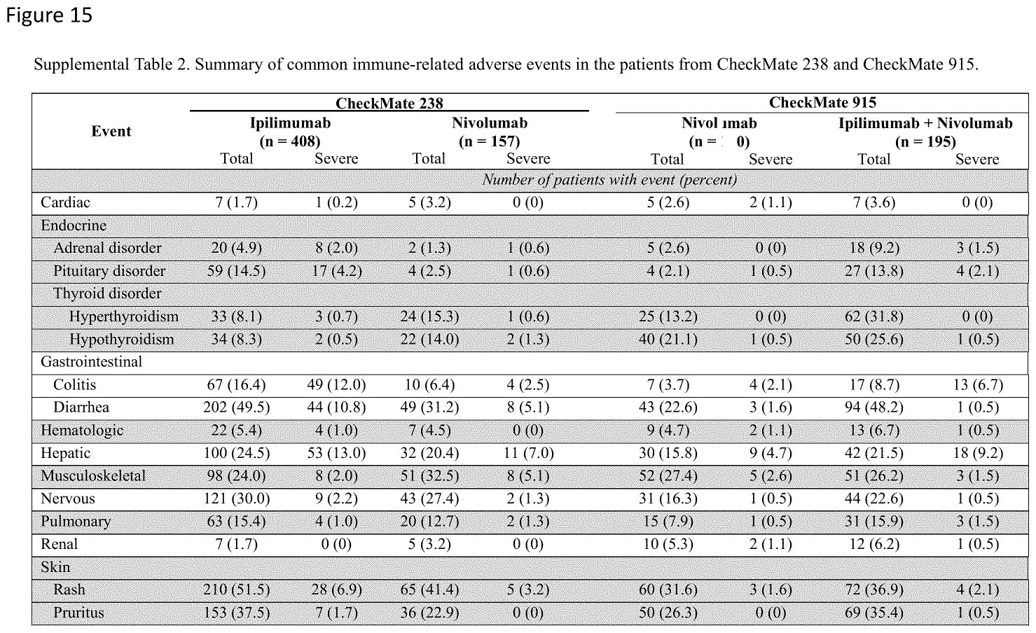

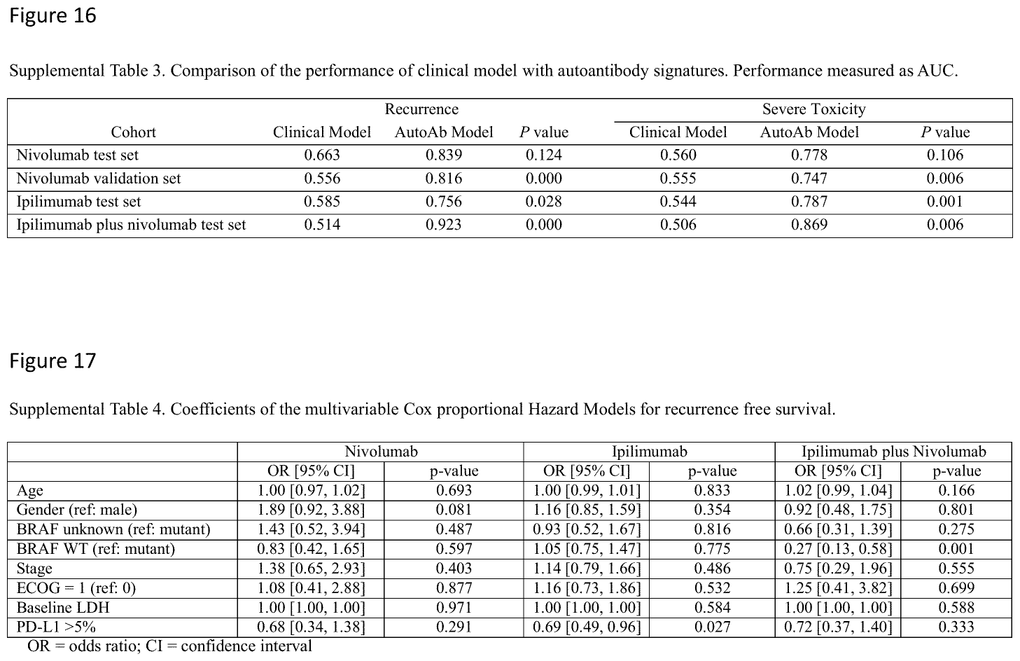

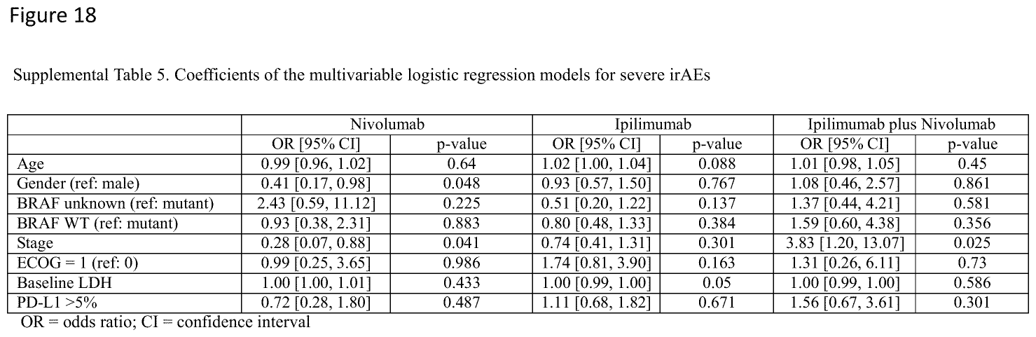

There was no significant overlap in the autoAbs that constituted the final prediction signatures (P>0.05 for all comparisons; FIG. 8). There was statistically significant, but numerically minimal, overlap between the DEAs associated with recurrence and severe toxicity for ipilimumab monotherapy as well as for combination ipilimumab plus nivolumab treatment. There was no significant overlap in the DEAs associated with nivolumab recurrence and severe toxicity (FIG. 9). In line with these findings, there was no significant correlation between the development of severe toxicity and recurrence for any of the three regimens (for ipilimumab patients, P=0.610; for nivolumab patients from CheckMate 238, P=0.773; for nivolumab patients from CheckMate 915, P=1.000; for ipilimumab plus nivolumab patients, P=0.574).

Given the similar clinical manifestations of immunotherapy toxicity and conventional autoimmune disorders, we next investigated whether the same autoAbs are associated with both processes. The HuProt microarray includes several established markers of autoimmune disease including dsDNA, JO-1, SCL-70, Smith (Sm) antigen, Sm/RNP complex, SSA, Lupus La protein (SSB), and thyroid peroxidase (TPO). SSB was found at higher levels in patients who received nivolumab and developed severe toxicity (P=0.0005), but there was otherwise no significant differential expression of these antibodies for any of the treatment-outcome combinations. We developed a more comprehensive list of autoAb commonly found in autoimmune disease but found that there was no significant enrichment for these antibodies. In an additional analysis of genes related to autoimmune disease pathogenesis, we found that the list of DEA for patients who received ipilimumab and developed severe toxicity was enriched for antigens related to autoimmune diseases (P=0.013). However, there was no significant enrichment for any of the other treatment/outcome combinations (P>0.15 for all remaining) [Supplemental Table 3].

For each treatment-outcome combination, we explored whether the profile of baseline DEA was enriched for IgGs targeting proteins linked to melanoma pathogenesis. We included in this analysis an additional 47 known melanoma autoantigens. None of the treatment-outcome combinations were enriched for antibodies to this set of melanoma related proteins and neoantigens (P>0.20 for all).

Finally, we explored whether any of the DEA targets were known elements of PD-1 or CTLA-4 signaling cascades. The DEA for ipilimumab efficacy was enriched for self-antigens related to CTLA4 blockade [CD80, CD86, PIK3CA, and PTPN11; P=0.001). The list of DEA in nivolumab patients with severe toxicity was enriched for self-antigens related to PD1 blockade [HLA-A, HLA-DRB1, NFAT5, and STAT3; P=0.038]. AutoAb to LAG3 and CTLA4 were expressed at significantly higher levels in patients who received ipilimumab plus nivolumab and did not recur (P=0.013 and P=0.023, respectively). There was no significant differential expression of autoantibodies to either LAG3 or CTLA4 for the other treatment-outcome combinations. Similarly, there was no significant differential expression of autoantibodies to PD-1 or PD-L1 for any of the treatment-outcome combinations.

### Discussion of Examples

In view of the foregoing, it will be recognized that the present disclosure provides a composite panel of baseline serum autoantibodies that predict disease recurrence and severe toxicity in patients with resected stage III or IV melanoma receiving adjuvant nivolumab, ipilimumab, or combination ipilimumab plus nivolumab. For each treatment regimen, the autoAb signatures could be used together to accurately stratify patients based on their projected likelihood of suffering disease recurrence and developing severe irAEs. This is in contrast with most biomarkers of immunotherapy response under active investigation, which aim to predict either treatment efficacy or toxicity. The ability to simultaneously forecast both outcomes would enable providers and patients to view the possibility of clinical benefit in the context of potential toxicity, and ultimately help optimize treatment regimens while minimizing exposure to severe irAEs.

Prior to the present disclosure, there were no robust tools for identifying melanoma patients at risk for disease recurrence or immunotoxicity after treatment with immune checkpoint inhibitors (Patil et al., 2018; von Itzstein et al., 2020). Pretreatment tumor cell expression of PD-L1 was one of the first and most extensively studied candidate biomarkers of ICB response (Gibney et al., 2016; Topalian et al., 2012). Although some data suggest that higher PD-L1 expression is associated with better treatment outcomes, PD-L1 status has limited predictive utility and many patients classified as negative still respond to anti-PD-1 therapy (Daud et al., 2016). We found that autoAb panels outperformed a model composed of clinical covariates including PD-L1 status. Compared to the clinical model, the parsimonious autoAb signatures predicted recurrence more accurately and with demonstrated net clinical benefit for all three treatment regimens.

The autoAb signatures identified in this disclosure offer several other advantages including: (1) they can be combined into a single assay that simultaneously yields recurrence and severe toxicity predictions for multiple treatments; (2) they can reliably predict recurrence for therapies targeting a wide spectrum of immune checkpoints, which stands in contrast to percent PD-L1 expression, whose use is limited to anti-PD-1 therapies (Postow et al., 2015); and (3) from a technical standpoint, the autoAb readouts and the signature algorithms can be applied to each patient using their own internal control, which is the median overall signal intensity on that individual's panel. This obviates the need to use external controls or normalizing processes when testing serum from new patients.

Certain aspects of the disclosure involve using only the predictive power of antibodies expressed at higher levels in patients without recurrence or with severe toxicity. This is because in part the production and presence of autoAbs indicates subclinical immune system activity, which in turn foreshadows the immune system activation seen in patients who respond to treatment or develop severe immunotoxicity. For the same reason, in embodiments, the disclosure does not limit the signature building process to autoAbs whose expression exceeded a select number of standard deviations above that of the healthy controls, which is frequently done in autoimmune disease antibody research (de Moel, Derksen, et al., 2019; Pardos-Gea et al., 2017).

In the present disclosure, the datasets come from two separate Phase III, randomized, controlled studies so the serum collection, treatment schedules, and outcomes assessments were all standardized. Although the cohort of patients from CheckMate 238 was intentionally chosen to be enriched for irAEs, the patients and their clinical outcomes from both CheckMate 238 and CheckMate 915 are otherwise representative of the overall trial populations. The disclosure also demonstrates that the autoAb signatures consistently outperform a model that includes percent PD-L1 expression, which is the current clinical benchmark for identifying patients likely to respond to immunotherapy. Further, since both CheckMate 238 and CheckMate 915 had an arm of patients treated with nivolumab, the disclosure includes validation of the chosen cutoffs for the recurrence and toxicity signatures on a held out independent validation dataset composed of all the patients from CheckMate 915.

The following reference list is not an indication that any of the references are material to patentability:
Barrow, C., Browning, J., MacGregor, D., Davis, I. D., Sturrock, S., Jungbluth, A. A., & Cebon, J. (2006). Tumor antigen expression in melanoma varies according to antigen and stage. Clin Cancer Res, 12(3 Pt 1), 764-771. https://doi.org/10.1158/1078-0432.CCR-05-1544
Bin Lim, S., Chua, M. L. K., Yeong, J. P. S., Tan, S. J., Lim, W. T., & Lim, C. T. (2019). Pan-cancer analysis connects tumor matrisome to immune response. NPJ Precis Oncol, 3, 15. https://doi.org/10.1038/s41698-019-0087-0
Das, R., Bar, N., Ferreira, M., Newman, A. M., Zhang, L., Bailur, J. K., Bacchiocchi, A., Kluger, H., Wei, W., Halaban, R., Sznol, M., Dhodapkar, M. V., & Dhodapkar, K. M. (2018). Early B cell changes predict autoimmunity following combination immune checkpoint blockade. J Clin Invest, 128(2), 715-720. https://doi.org/10.1172/JCI96798
Daud, A. I., Wolchok, J. D., Robert, C., Hwu, W. J., Weber, J. S., Ribas, A., Hodi, F. S., Joshua, A. M., Kefford, R., Hersey, P., Joseph, R., Gangadhar, T. C., Dronca, R., Patnaik, A., Zarour, H., Roach, C., Toland, G., Lunceford, J. K., Li, X. N., Emancipator, K., Dolled-Filhart, M., Kang, S. P., Ebbinghaus, S., & Hamid, O. (2016). Programmed Death-Ligand 1 Expression and Response to the Anti-Programmed Death 1 Antibody Pembrolizumab in Melanoma. J Clin Oncol, 34(34), 4102-4109. https://doi.org/10.1200/JCO.2016.67.2477
de Moel, E. C., Derksen, V. F. A. M., Trouw, L. A., Bang, H., Collée, G., Lard, L. R., Ramiro, S., Huizinga, T. W. J., Allaart, C. F., Toes, R. E. M., & van der Woude, D. (2019). In rheumatoid arthritis, changes in autoantibody levels reflect intensity of immunosuppression, not subsequent treatment response. Arthritis Res Ther, 21(1), 28. https://doi.org/10.1186/s13075-019-1815-0
de Moel, E. C., Rozeman, E. A., Kapiteijn, E. H., Verdegaal, E. M. E., Grummels, A., Bakker, J. A., Huizinga, T. W. J., Haanen, J. B., Toes, R. E. M., & van der Woude, D. (2019). Autoantibody Development under Treatment with Immune-Checkpoint Inhibitors. Cancer Immunol Res, 7(1), 6-11. https://doi.org/10.1158/2326-6066.CIR-18-0245
Diem, S., Fässler, M., Bomze, D., Ali, 0. H., Berner, F., Niederer, R., Hillmann, D., Mangana, J., Levesque, M. P., Dummer, R., Risch, L., Recher, M., Risch, M., & Flatz, L. (2019). Immunoglobulin G and Subclasses as Potential Biomarkers in Metastatic Melanoma Patients Starting Checkpoint Inhibitor Treatment. J Immunother, 42(3), 89-93. https://doi.org/10.1097/CJI.0000000000000255
Eggermont, A. M., Chiarion-Sileni, V., Grob, J. J., Dummer, R., Wolchok, J. D., Schmidt, H., Hamid, O., Robert, C., Ascierto, P. A., Richards, J. M., Lebbé, C., Ferraresi, V., Smylie, M., Weber, J. S., Maio, M., Bastholt, L., Mortier, L., Thomas, L., Tahir, S., Hauschild, A., Hassel, J. C., Hodi, F. S., Taitt, C., de Pril, V., de Schaetzen, G., Suciu, S., & Testori, A. (2016). Prolonged Survival in Stage III Melanoma with Ipilimumab Adjuvant Therapy. N Engl J Med, 375(19), 1845-1855. https://doi.org/10.1056/NEJMoa1611299
Fässler, M., Diem, S., Mangana, J., Hasan Ali, O., Berner, F., Bomze, D., Ring, S., Niederer, R., Del Carmen Gil Cruz, C., Perez Shibayama, C. I., Krolik, M., Siano, M., Joerger, M., Recher, M., Risch, L., Güsewell, S., Risch, M., Speiser, D. E., Ludewig, B., Levesque, M. P., Dummer, R., & Flatz, L. (2019). Antibodies as biomarker candidates for response and survival to checkpoint inhibitors in melanoma patients. J Immunother Cancer, 7(1), 50. https://doi.org/10.1186/s40425-019-0523-2
Gibney, G. T., Weiner, L. M., & Atkins, M. B. (2016). Predictive biomarkers for checkpoint inhibitor-based immunotherapy. Lancet Oncol, 17(12), e542-e551. https://doi.org/10.1016/S1470-2045(16)30406-5
Gilbert, A. E., Karagiannis, P., Dodev, T., Koers, A., Lacy, K., Josephs, D. H., Takhar, P., Geh, J. L., Healy, C., Harries, M., Acland, K. M., Rudman, S. M., Beavil, R. L., Blower, P. J., Beavil, A. J., Gould, H. J., Spicer, J., Nestle, F. O., & Karagiannis, S. N. (2011). Monitoring the systemic human memory B cell compartment of melanoma patients for anti-tumor IgG antibodies. PLoS One, 6(4), e19330. https://doi.org/10.1371/journal.pone.0019330
Gowen, M. F., Giles, K. M., Simpson, D., Tchack, J., Zhou, H., Moran, U., Dawood, Z., Pavlick, A. C., Hu, S., Wilson, M. A., Zhong, H., Krogsgaard, M., Kirchhoff, T., & Osman, I. (2018). Baseline antibody profiles predict toxicity in melanoma patients treated with immune checkpoint inhibitors. J Transl Med, 16(1), 82. https://doi.org/10.1186/s12967-018-1452-4
Griss, J., Bauer, W., Wagner, C., Simon, M., Chen, M., Grabmeier-Pfistershammer, K., Maurer-Granofszky, M., Roka, F., Penz, T., Bock, C., Zhang, G., Herlyn, M., Glatz, K., Läubli, H., Mertz, K. D., Petzelbauer, P., Wiesner, T., Hartl, M., Pickl, W. F., Somasundaram, R., Steinberger, P., & Wagner, S. N. (2019). B cells sustain inflammation and predict response to immune checkpoint blockade in human melanoma. Nat Commun, 10(1), 4186. https://doi.org/10.1038/s41467-019-12160-2
Helmink, B. A., Reddy, S. M., Gao, J., Zhang, S., Basar, R., Thakur, R., Yizhak, K., Sade-Feldman, M., Blando, J., Han, G., Gopalakrishnan, V., Xi, Y., Zhao, H., Amaria, R. N., Tawbi, H. A., Cogdill, A. P., Liu, W., LeBleu, V. S., Kugeratski, F. G., Patel, S., Davies, M. A., Hwu, P., Lee, J. E., Gershenwald, J. E., Lucci, A., Arora, R., Woodman, S., Keung, E. Z., Gaudreau, P. O., Reuben, A., Spencer, C. N., Burton, E. M., Haydu, L. E., Lazar, A. J., Zapassodi, R., Hudgens, C. W., Ledesma, D. A., Ong, S., Bailey, M., Warren, S., Rao, D., Krijgsman, O., Rozeman, E. A., Peeper, D., Blank, C. U., Schumacher, T. N., Butterfield, L. H., Zelazowska, M. A., McBride, K. M., Kalluri, R., Allison, J., Petitprez, F., Fridman, W. H., Sautès-Fridman, C., Hacohen, N., Rezvani, K., Sharma, P., Tetzlaff, M. T., Wang, L., & Wargo, J. A. (2020). B cells and tertiary lymphoid structures promote immunotherapy response. Nature, 577(7791), 549-555. https://doi.org/10.1038/s41586-019-1922-8
Hodi, F. S. (2006). Well-defined melanoma antigens as progression markers for melanoma: insights into differential expression and host response based on stage. Clin Cancer Res, 12(3 Pt 1), 673-678. https://doi.org/10.1158/1078-0432.CCR-05-2616
Jeong, J. S., Jiang, L., Albino, E., Marrero, J., Rho, H. S., Hu, J., Hu, S., Vera, C., Bayron-Poueymiroy, D., Rivera-Pacheco, Z. A., Ramos, L., Torres-Castro, C., Qian, J., Bonaventura, J., Boeke, J. D., Yap, W. Y., Pino, I., Eichinger, D. J., Zhu, H., & Blackshaw, S. (2012). Rapid identification of monospecific monoclonal antibodies using a human proteome microarray. Mol Cell Proteomics, 11(6), 0111.016253. https://doi.org/10.1074/mcp.0111.016253
Jäger, E., Gnjatic, S., Nagata, Y., Stockert, E., Jäger, D., Karbach, J., Neumann, A., Rieckenberg, J., Chen, Y. T., Ritter, G., Hoffman, E., Arand, M., Old, L. J., & Knuth, A. (2000). Induction of primary NY-ESO-1 immunity: CD8+ T lymphocyte and antibody responses in peptide-vaccinated patients with NY-ESO-1+ cancers. Proc Natl Acad Sci USA, 97(22), 12198-12203. https://doi.org/10.1073/pnas.220413497
Kimbara, S., Fujiwara, Y., Iwama, S., Ohashi, K., Kuchiba, A., Arima, H., Yamazaki, N., Kitano, S., Yamamoto, N., & Ohe, Y. (2018). Association of antithyroglobulin antibodies with the development of thyroid dysfunction induced by nivolumab. Cancer Sci, 109(11), 3583-3590. https://doi.org/10.1111/cas.13800
McGranahan, N., Furness, A. J., Rosenthal, R., Ramskov, S., Lyngaa, R., Saini, S. K., Jamal-Hanjani, M., Wilson, G. A., Birkbak, N. J., Hiley, C. T., Watkins, T. B., Shafi, S., Murugaesu, N., Mitter, R., Akarca, A. U., Linares, J., Marafioti, T., Henry, J. Y., Van Allen, E. M., Miao, D., Schilling, B., Schadendorf, D., Garraway, L. A., Makarov, V., Rizvi, N. A., Snyder, A., Hellmann, M. D., Merghoub, T., Wolchok, J. D., Shukla, S. A., Wu, C. J., Peggs, K. S., Chan, T. A., Hadrup, S. R., Quezada, S. A., & Swanton, C. (2016). Clonal neoantigens elicit T cell immunoreactivity and sensitivity to immune checkpoint blockade. Science, 351(6280), 1463-1469. https://doi.org/10.1126/science.aaf1490 Michot, J. M., Bigenwald, C., Champiat, S., Collins, M., Carbonnel, F., Postel-Vinay, S., Berdelou, A., Varga, A., Bahleda, R., Hollebecque, A., Massard, C., Fuerea, A., Ribrag, V., Gazzah, A., Armand, J. P., Amellal, N., Angevin, E., Noel, N., Boutros, C., Mateus, C., Robert, C., Soria, J. C., Marabelle, A., & Lambotte, 0. (2016). Immune-related adverse events with immune checkpoint blockade: a comprehensive review. Eur J Cancer, 54, 139-148. https://doi.org/10.1016/j.ejca.2015.11.016
Mushtaq, M. U., Papadas, A., Pagenkopf, A., Flietner, E., Morrow, Z., Chaudhary, S. G., & Asimakopoulos, F. (2018). Tumor matrix remodeling and novel immunotherapies: the promise of matrix-derived immune biomarkers. J Immunother Cancer, 6(1), 65. https://doi.org/10.1186/s40425-018-0376-0
Pardos-Gea, J., Cortés-Hernández, J., Castro-Marrero, J., Balada, E., & Ordi-Ros, J. (2017). Autoantibodies to types I and IV collagen and heart valve disease in systemic lupus erythematosus/antiphospholipid syndrome. Clin Rheumatol, 36(6), 1401-1406. https://doi.org/10.1007/s10067-017-3594-9
Patil, P. D., Burotto, M., & Velcheti, V. (2018). Biomarkers for immune-related toxicities of checkpoint inhibitors: current progress and the road ahead. Expert Rev Mol Diagn, 18(3), 297-305. https://doi.org/10.1080/14737159.2018.1440209
Postow, M. A., Chesney, J., Pavlick, A. C., Robert, C., Grossmann, K., McDermott, D., Linette, G. P., Meyer, N., Giguere, J. K., Agarwala, S. S., Shaheen, M., Ernstoff, M. S., Minor, D., Salama, A. K., Taylor, M., Ott, P. A., Rollin, L. M., Horak, C., Gagnier, P., Wolchok, J. D., & Hodi, F. S. (2015). Nivolumab and ipilimumab versus ipilimumab in untreated melanoma. N Engl J Med, 372(21), 2006-2017. https://doi.org/10.1056/NEJMoa1414428
Postow, M. A., Sidlow, R., & Hellmann, M. D. (2018). Immune-Related Adverse Events Associated with Immune Checkpoint Blockade. N Engl J Med, 378(2), 158-168. https://doi.org/10.1056/NEJMra1703481
Tahir, S. A., Gao, J., Miura, Y., Blando, J., Tidwell, R. S. S., Zhao, H., Subudhi, S. K., Tawbi, H., Keung, E., Wargo, J., Allison, J. P., & Sharma, P. (2019). Autoimmune antibodies correlate with immune checkpoint therapy-induced toxicities. Proc Natl Acad Sci USA, 116(44), 22246-22251. https://doi.org/10.1073/pnas.1908079116
Tio, D., Kasiem, F. R., Willemsen, M., van Doom, R., van der Werf, N., Hoekzema, R., Luiten, R. M., & Bekkenk, M. W. (2019). Expression of cancer/testis antigens in cutaneous melanoma: a systematic review. Melanoma Res, 29(4), 349-357. https://doi.org/10.1097/CMR.0000000000000569
Toi, Y., Sugawara, S., Sugisaka, J., Ono, H., Kawashima, Y., Aiba, T., Kawana, S., Saito, R., Aso, M., Tsurumi, K., Suzuki, K., Shimizu, H., Domeki, Y., Terayama, K., Nakamura, A., Yamanda, S., Kimura, Y., & Honda, Y. (2019). Profiling Preexisting Antibodies in Patients Treated With Anti-PD-1 Therapy for Advanced Non-Small Cell Lung Cancer. JAMA Oncol, 5(3), 376-383. https://doi.org/10.1001/jamaonco1.2018.5860
Topalian, S. L., Hodi, F. S., Brahmer, J. R., Gettinger, S. N., Smith, D. C., McDermott, D. F., Powderly, J. D., Carvajal, R. D., Sosman, J. A., Atkins, M. B., Leming, P. D., Spigel, D. R., Antonia, S. J., Horn, L., Drake, C. G., Pardoll, D. M., Chen, L., Sharfman, W. H., Anders, R. A., Taube, J. M., McMiller, T. L., Xu, H., Korman, A. J., Jure-Kunkel, M., Agrawal, S., McDonald, D., Kollia, G. D., Gupta, A., Wigginton, J. M., & Sznol, M. (2012). Safety, activity, and immune correlates of anti-PD-1 antibody in cancer. N Engl J Med, 366(26), 2443-2454. https://doi.org/10.1056/NEJMoa1200690
von Itzstein, M. S., Khan, S., & Gerber, D. E. (2020). Investigational Biomarkers for Checkpoint Inhibitor Immune-Related Adverse Event Prediction and Diagnosis. Clin Chem, 66(6), 779-793. https://doi.org/10.1093/clinchem/hvaa081
Weber, J., Mandala, M., Del Vecchio, M., Gogas, H. J., Arance, A. M., Cowey, C. L., Dalle, S., Schenker, M., Chiarion-Sileni, V., Marquez-Rodas, I., Grob, J. J., Butler, M. O., Middleton, M. R., Maio, M., Atkinson, V., Queirolo, P., Gonzalez, R., Kudchadkar, R. R., Smylie, M., Meyer, N., Mortier, L., Atkins, M. B., Long, G. V., Bhatia, S., Lebbé, C., Rutkowski, P., Yokota, K., Yamazaki, N., Kim, T. M., de Pril, V., Sabater, J., Qureshi, A., Larkin, J., Ascierto, P. A., & Collaborators, C. (2017). Adjuvant Nivolumab versus Ipilimumab in Resected Stage III or IV Melanoma. N Engl J Med, 377(19), 1824-1835. https://doi.org/10.1056/NEJMoa1709030
Weinmann, S. C., & Pisetsky, D. S. (2019). Mechanisms of immune-related adverse events during the treatment of cancer with immune checkpoint inhibitors. Rheumatology (Oxford), 58(Suppl 7), vii59-vii67. https://doi.org/10.1093/rheumatology/kez308
Tibshirani R. Regression shrinkage and selection via the lasso. J Royal Stat Soc Series B (Methodological) 1996; 58(1):267-288
Meinshausen N, Bühlmann P. Stability selection. J Royal Stat Soc: Series B (Statistical Methodology) 2010; 72(4):417-473
Federico A, Monti S (2020). “hypeR: an R package for geneset enrichment workflows.” Bioinformatics, 36(4), 1307-1308.
DeLong, E. R., et al. (1988). Comparing the Areas under Two or More Correlated Receiver Operating Characteristic Curves: A Nonparametric Approach.

What is claimed is:

1. A method for predicting disease recurrence and toxicity for immune checkpoint inhibitor therapy in an individual, the method comprising determining an amount of a plurality of baseline autoantibodies (autoAbs) from a biological sample obtained from the individual, and comparing the amount of baseline autoAbs from the biological sample to a reference, wherein determining a difference between the baseline autoAbs from the biological sample to the reference provides an indication of whether or not the individual will have disease recurrence and toxicity in response to immune checkpoint inhibitor therapy that may be administered to the individual subsequent to obtaining the biological sample.

2. The method of claim 1, wherein the individual has been diagnosed with melanoma.

3. The method of claim 1, wherein the immune checkpoint inhibitor therapy comprises use of a checkpoint inhibitor monotherapy with an anti-PD-1 antibody, or use of a checkpoint inhibitor monotherapy with an anti-CTLA-4 antibody, or use of a combination checkpoint inhibitor therapy with the anti-PD-1 antibody and the anti-CTLA-4 antibody.

4. The method of claim 3, wherein the anti-PD-1 antibody comprises Nivolumab, and/or the anti-CTLA-4 antibody comprises Ipilimumab.

5. The method of claim 1, wherein the immune checkpoint inhibitor therapy comprises:
i) a checkpoint inhibitor monotherapy with ipilimumab;
ii) a checkpoint inhibitor monotherapy with nivolumab; or
iii) a combination checkpoint inhibitor with ipilimumab and nivolumab.

6. The method of claim 5, further comprising one or more of the following:
a) determining autoAbs that bind to one or more proteins of Table A to predict recurrence of the cancer after treatment with Ipilimumab, and optionally, if the recurrence is predicted, treating the patient with at least one anti-cancer agent that is not Ipilimumab;
b) determining autoAbs that bind to one or more proteins of Table B to predict toxicity to treatment with Ipilimumab, and optionally, if toxicity is predicted, administering one or more agents to the patient to reduce the toxicity, and if toxicity and recurrence is not predicted, treating the patient with the Ipilimumab;
c) determining autoAbs that bind to one or more proteins of Table C to predict recurrence of the cancer after treatment with Nivolumab, and optionally, if the recurrence is predicted, treating the patient with at least one anti-cancer agent that is not Nivolumab;
d) determining autoAbs that bind to one or more proteins of Table D to predict toxicity to treatment with Nivolumab; and optionally, if toxicity is predicted, administering one or more agents to the patient to reduce the toxicity, and if toxicity and recurrence is not predicted, treating the patient with the Nivolumab;
e) determining autoAbs that bind to one or more proteins of Table D to predict recurrence of the cancer after treatment with a combination of Ipilimumab and Nivolumab, and optionally, if the recurrence is predicted, treating the patient with at least one anti-cancer agent that does not include the combination of Ipilimumab and Nivolumab;
f) determining autoAbs that bind to one or more proteins of Table D to predict toxicity to treatment with the combination of Ipilimumab and Nivolumab, and optionally, if toxicity is predicted, administering one or more agents to the patient to reduce the toxicity, and if toxicity and recurrence is not predicted, treating the patient with the combination of Ipilimumab and Nivolumab.

7. The method of claim 6, wherein the patient has melanoma.

8. A substrate comprising a plurality of proteins from Table X, wherein each protein in the plurality of proteins is reversibly or irreversibly attached to the substrate.

9. The substrate of claim 8, where the plurality of proteins attached to the substrate comprises fewer than 21,000 proteins.

10. The substrate of claim 9, wherein the only proteins in the plurality of proteins attached to the substrate are selected from the proteins of Table X.

11. The substrate of claim 9, wherein the proteins are in contact with a sample from an individual who has cancer.

12. The substrate of claim 11, wherein the individual has melanoma.

13. The substrate of claim 12, wherein auto-antibodies in the patient sample are bound to at least some of the proteins on the substrate.
